(19) United States
(12) Patent Application Publication
(10) Pub. No.: US 2007/0213359 A1
(43) Pub. Date: Sep. 13, 2007

(54) BICYCLIC-NITROGEN COMPOUNDS AS MODULATORS OF GHRELIN RECEPTOR AND USES THEREOF

(75) Inventors: Ethan S. Burstein, San Diego, CA (US); Anne Eeg Knapp, Frederiksberg C. (DK); Roger Olsson, Bunkeflostrand (SE); Jorgen Eskildsen, Copenhagen (DK); Fredrik Ek, Lund (SE)

Correspondence Address: KNOBBE MARTENS OLSON & BEAR LLP, 2040 MAIN STREET, FOURTEENTH FLOOR, IRVINE, CA, 92614, US

(73) Assignee: ACADIA Pharmaceuticals Inc., San Diego, CA (US)

(21) Appl. No.: 11/618,724

(22) Filed: Dec. 29, 2006

Related U.S. Application Data
(60) Provisional application No. 60/755,714, filed on Dec. 30, 2005.
(60) Provisional application No. 60/835,241, filed on Aug. 2, 2006.

(51) Int. Cl. C07D 471/02; A61K 31/4745; A61K 31/405; C07D 403/02

(52) U.S. Cl. 514/300; 514/419; 546/113; 548/465; 548/495; 514/414

(57) ABSTRACT

Disclosed herein are compounds of Formula I 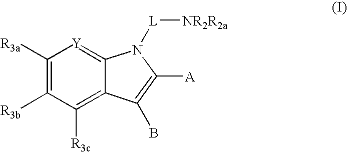 as defined herein, or a pharmaceutically acceptable salt, ester, amide, or prodrug thereof, that modulates the activity of a ghrelin receptor. Disclosed herein are also methods of treating diseases or conditions that comprise administering to a subject in need thereof a therapeutically effective amount of a compound of Formula I.

### CROSS-REFERENCE TO RELATED APPLICATIONS

This application claims priority to U.S. Provisional Application Nos. 60/755,714, entitled “INDOLE COMPOUNDS AS MODULATORS OF GHRELIN RECEPTOR AND USES THEREOF”, filed Dec. 30, 2005; and 60/835,241, entitled “INDOLE COMPOUNDS AS MODULATORS OF GHRELIN RECEPTOR AND USES THEREOF, filed Aug. 2, 2006, both of which are incorporated by reference in their entireties.

### BACKGROUND OF THE INVENTION

1. Field of the Invention

This invention relates to the fields of organic chemistry, pharmaceutical chemistry, biochemistry, molecular biology and medicine. In particular it relates to compounds that modulate the activity of the human Growth Hormone Secretagogue receptor (GHSR1a, Ghrelin receptor), and to the use of the compounds for the treatment and prevention of disorders or conditions such as obesity, eating disorders, hormone insufficiencies, dwarfism; somatopause, osteoporosis, wasting syndromes, catabolic states, cardiovascular diseases, gastrointestinal diseases, sleep disorders, cancers; for disorders of the pancreas, diabetes, anxiety disorders and cognitive deficits, and for diagnosing hormone insufficiencies.

2. Description of the Related Art

The physiological actions of the hormone/neurotransmitter ghrelin are mediated, in part, by the ghrelin receptor. The ghrelin receptor is expressed in a number of tissues including the pituitary and hypothalamus, as well as other brain regions such as hippocampus, as well as peripheral tissues such as heart, lung, pancreas, stomach, intestine, and adipose tissue and numerous other tissues where it is thought to regulate appetite, energy balance, cardiovascular function, gastrointestinal motility, hormone release, induction of slow wave sleep, and cellular proliferation (Inui A, et al. FASEB J. 2004 March; 18(3):439-56. Deghenghi R. et al. Endocrine. 2003 October; 22(1):13-8. Bona G et al. Panminerva Med. 2003 September; 45(3):197-201. Broglio F. et al. Horm Res. 2003; 59(3):109-17).

Compounds that stimulate ghrelin receptors have been shown to stimulate appetite and food intake, improve cardiac output and reduce cardiac afterload, stimulate gastric motility and emptying, facilitate induction of sleep, and inhibit cellular proliferation in cells derived from the lung, thyroid and breast. Compounds that block ghrelin receptor activity have been shown to facilitate weight loss, reduce appetite, reduce food intake, facilitate weight maintenance, treat obesity, treat diabetes and associated side effects, (including retinopathy and/or cardiovascular disorders), and reduce metabolism. (Inui A, et al. FASEB J. 2004 March; 18(3):439-56. Deghenghi R. et al. Endocrine. 2003 October; 22(1):13-8. Bona G et al. Panminerva Med. 2003 September; 45(3):197-201. Broglio F. et al. Horm Res. 2003; 59(3): 109-17).

### SUMMARY OF THE INVENTION

One embodiment disclosed herein relates to a compound of Formula (I): 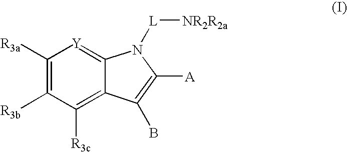
or a solvate, a polymorph, a metabolite, or a pharmaceutically acceptable salt or prodrug thereof, wherein:

A can be selected from the group consisting of hydrogen, halogen, cyano, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{6b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(═O)R₁;

B can be selected from the group consisting of hydrogen; mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, —C(=Z)R₁, —C(=Z)OR₁, —C(=Z)NR₁ₐR_{1b}, —C(=Z)N(OR₁ₐ)R_{1b}, —C(=Z)N(R₁)NR₁ₐR_{1b}, —C(R₁)═NR₁ₐ, —C≡N; —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, —OR₁, —SR₁, and —OC(═O)R₁; or

A and B can be taken together to form an unsubstituted or substituted cycloalkyl, or unsubstituted or substituted heteroalicyclyl;

R₁, R₁ₐ and R_{1b} can each independently selected from the group consisting of hydrogen, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl and haloalkyl;

R₂ and R₂ₐ can each independently selected from the group consisting of hydrogen, cyano, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, sulfinyl, sulfonyl, haloalkyl, —C(=Z)R₁, —C(=Z)OR₁, —C(=Z)NR₁ₐR_{1b}, —C(R₁)═NR₁ₐ, —(C₁₋₄alkyl)-Z-aryl, —(C₁₋₄alkyl)C(═O) R₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁; or

R₂ and R₂ₐ can be taken together, along with the nitrogen atom to which they are attached, to form an unsubstituted or substituted heteroalicyclyl;

R₃, R₃ₐ, R_{3b}, and R_{3c} can each independently selected from the group consisting of hydrogen, halogen, cyano, nitro, mono-substituted, poly-substituted or unsubstituted variants of the following residues: alkyl, alkenyl, alkynyl, cycloalkyl, cycloalkenyl cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

R₃, R₃ₐ, R_{3b}, and R_{3c} can be taken together with one or more adjacent members of the group consisting of R₃, R₃ₐ, R_{3b}, and R_{3c} to form a cycloalkyl, cycloalkenyl, cycloalkynyl, or heteroalicyclyl ring;

R_{3c} can be taken together with B to form a cycloalkyl, cycloalkenyl, cycloalkynyl, or heteroalicyclyl ring;

L can be an unsubstituted or substituted lower alkylene group, wherein when L is substituted, it is substituted with one or more group(s) individually and independently selected from the group consisting of alkyl, alkenyl, halogen, haloalkyl, alkoxy, haloalkoxy, hydroxyl, and —CN;

L can be taken together with R₃ to form a cycloalkyl, cycloalkenyl, cycloalkynyl, or heteroalicyclyl ring;

Y can be C—R₃ or N; and

Z can be O or S.

Another embodiment disclosed herein relates to a pharmaceutical composition, comprising a therapeutically effective amount of a compound of Formula (I) and/or a compound described herein, and a pharmaceutically acceptable carrier, excipient, or diluent.

Still another embodiment disclosed herein relates to a method of treating or preventing a disorder or condition comprising administering to a subject a pharmaceutically effective amount of a compound of Formula (I) and/or a compound described herein. In some embodiment, the compound of Formula (I) and/or one of the compound described herein alleviates or treats a disorder or condition by modulating, agonizing, inverse agonizing, or antagonizing a ghrelin receptor.

### BRIEF DESCRIPTION OF THE DRAWINGS

FIG. 1 is a graph showing the inverse agonists activities of compounds at ghrelin receptors in R-SAT assays.

FIG. 2 is a graph showing the inverse agonist and agonist activities of compounds at ghrelin receptors in phosphatidyl inositol assays

FIG. 3 is a graph sho222wing the spontaneous feeding activity in freely moving, fasted, male Sprague-Dawley rats following intraperitoneal administration of ghrelin receptor antagonists/inverse agonists.

### DETAILED DESCRIPTION OF THE PREFERRED EMBODIMENT

Small molecules with heretofore-unappreciated activities have been identified as ghrelin receptor antagonists/inverse agonists. We further demonstrate that these compounds suppress feeding in rats. These observations have practical applications that support the use of these compounds to alleviate or treat disorders or conditions affected directly or indirectly through ghrelin receptors.

A large number of compounds of Formula I were screened for functional activity at the Ghrelin receptor and display robust ghrelin receptor antagonist/inverse agonist activity.

One embodiment disclosed herein relates to a compound of Formula (I): 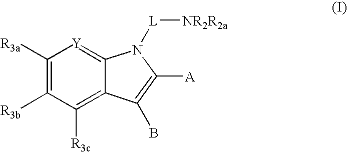
or a solvate, a polymorph, a metabolite, or a pharmaceutically acceptable salt or prodrug thereof, wherein:

A can be selected from the group consisting of hydrogen, halogen, cyano, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(═O)R₁;

B can be selected from the group consisting of hydrogen; mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, —C(=Z)R₁, —C(=Z)OR₁, —C(=Z)NR₁ₐR_{1b}, —C(=Z)N(OR₁ₐ)R_{1b}, —C(=Z)N(R₁)NR₁ₐR_{1b}, —C(R₁)═NR₁ₐ, —C≡N; —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, —OR₁, —SR₁, and —OC(═O)R₁; or

A and B can be taken together to form an unsubstituted or substituted cycloalkyl, or unsubstituted or substituted heteroalicyclyl;

R₁, R₁ₐ and R_{1b} can each independently selected from the group consisting of hydrogen, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl and haloalkyl;

R₂ and R₂ₐ can each independently selected from the group consisting of hydrogen, cyano, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, sulfinyl, sulfonyl, haloalkyl, —C(=Z)R₁, —C(=Z)OR₁, —C(=Z)NR₁ₐR_{1b}, —C(R₁)═NR₁ₐ, —(C₁₋₄alkyl)-Z-aryl, —(C₁₋₄alkyl)C(═O) R₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(O)R₁, —N(R₁)—S(═O)₂R₁, —OR₁, —SR₁, and —OC(=Z)R₁; or

R₂ and R₂ₐ can be taken together, along with the nitrogen atom to which they are attached, to form an unsubstituted or substituted heteroalicyclyl;

R₃, R₃ₐ, R_{3b}, and R_{3c} can each independently selected from the group consisting of hydrogen, halogen, cyano, nitro, mono-substituted, poly-substituted or unsubstituted variants of the following residues: alkyl, alkenyl, alkynyl, cycloalkyl, cycloalkenyl cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;

R₃, R₃ₐ, R_{3b}, and R_{3c} can be taken together with one or more adjacent members of the group consisting of R₃, R₃ₐ, R_{3b}, and R₃, to form a cycloalkyl, cycloalkenyl, cycloalkynyl, or heteroalicyclyl ring;

R_{3c} can be taken together with B to form a cycloalkyl, cycloalkenyl, cycloalkynyl, or heteroalicyclyl ring;

L can be an unsubstituted or substituted lower alkylene group, wherein when L is substituted, it is substituted with one or more group(s) individually and independently selected from the group consisting of alkyl, alkenyl, halogen, haloalkyl, alkoxy, haloalkoxy, hydroxyl, and —CN;

L can be taken together with R₃ to form a cycloalkyl, cycloalkenyl, cycloalkynyl, or heteroalicyclyl ring;

Y can be C—R₃ or N; and

Z can be O or S.

In some embodiments, the compound of Formula (I) or a solvate, a polymorph, a metabolite, or a pharmaceutically acceptable salt or prodrug thereof, has the structure described herein provided that when R₂ and R₂ₐ are taken together, along with the nitrogen atom to which they are attached, form a substituted heteroalicyclyl, wherein the substituted heteroalicyclyl is 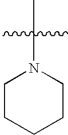
substituted with n-butyl at the para-position, then B cannot be selected from the group consisting of methyl, —C(═O)R₁, and —CH₂OH, wherein R₁ is hydrogen or methyl; or A cannot be methyl. In other embodiments, the compound of Formula (I) or a solvate, a polymorph, a metabolite, or a pharmaceutically acceptable salt or prodrug thereof, has the structure described herein provided that when R₂ and R₂ₐ are taken together, along with the nitrogen atom to which they are attached, form a substituted heteroalicyclyl, wherein the substituted heteroalicyclyl is 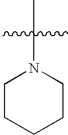
substituted with an alkyl, such as n-butyl, then A, R₃, R₃ₐ, R_{3b}, and R_{3c} cannot all be hydrogen.

Another embodiment disclosed herein relates to a compound of Formula (I) that modulates, agonizes, inverse agonizes, or antagonizes a ghrelin receptor. In some embodiments, a compound of Formula (I) inverse agonizes or antagonizes a ghrelin receptor. In some embodiments, the compound of Formula (I) binds to a ghrelin receptor with an ICSO value in the range of about 9 to about 5. In certain embodiments, the compound of Formula (I) binds to a ghrelin receptor with an IC₅₀ value in the range of about 9 to about 6. In some embodiments, the compound of Formula (I) binds to a ghrelin receptor with an IC₅₀ value in the range of about 9 to about 7. In certain embodiments, the compound of Formula (I) binds to a ghrelin receptor with an IC₅₀ value in the range of about 9 to about 8.

In some embodiments, Y can be C—R₃. In other embodiments, Y can be N (nitrogen).

With respect to R₂ and R₂ₐ, in some embodiments, R₂, and/or R₂ₐ can be hydrogen. In other embodiments, R₂, and/or R₂ₐ can be cyano. In still other embodiments, R₂, and/or R₂ₐ can be a mono-substituted, poly-substituted or unsubstituted alkyl. In yet other embodiments, R₂, and/or R₂ₐ can be a mono-substituted, poly-substituted or unsubstituted alkenyl. In some embodiments, R₂, and/or R₂ₐ can be a mono-substituted, poly-substituted or unsubstituted alkynyl. In other embodiments, R₂, and/or R₂ₐ can be a mono-substituted, poly-substituted or unsubstituted cycloalkyl. In yet other embodiments, R₂, and/or R₂ₐ can be a mono-substituted, poly-substituted or unsubstituted cycloalkenyl. In still other embodiments, R₂, and/or R₂ₐ can be a mono-substituted, poly-substituted or unsubstituted cycloalkynyl. In still other embodiments, R₂, and/or R₂ₐ can be a mono-substituted, poly-substituted or unsubstituted aryl. In some embodiments, R₂, and/or R₂ₐ can be a mono-substituted, poly-substituted or unsubstituted heteroaryl. In other embodiments, R₂, and/or R₂ₐ can be a mono-substituted, poly-substituted or unsubstituted heteroalicyclyl. In yet other embodiments, R₂, and/or R₂ₐ can be a mono-substituted, poly-substituted or unsubstituted aralkyl. In still other embodiments, R₂, and/or R₂ₐ can be a mono-substituted, poly-substituted or unsubstituted heteroaralkyl. In yet other embodiments, R₂, and/or R₂ₐ can be a mono-substituted, poly-substituted or unsubstituted (heteroalicyclyl)alkyl. In some embodiments, R₂, and/or R₂ₐ can be a mono-substituted, poly-substituted or unsubstituted —C(=Z)R₁. In other embodiments, R₂, and/or R₂ₐ can be a mono-substituted, poly-substituted or unsubstituted —C(=Z)OR₁. In other embodiments, R₂, and/or R₂ₐ can be a mono-substituted, poly-substituted or unsubstituted-C(=Z)NR₁ₐR_{1b}. In some embodiments, R₂, and/or R₂ₐ can be a mono-substituted, poly-substituted or unsubstituted —C(R₁)═NR₁ₐ. In other embodiments, R₂, and/or R₂ₐ can be a mono-substituted, poly-substituted or unsubstituted —NR₁ₐR_{1b}. In still other embodiments, R₂, and/or R₂ₐ can be a mono-substituted, poly-substituted or unsubstituted —N═CR₁ₐR_{1b}. In some embodiments, R₂, and/or R₂ₐ can be a mono-substituted, poly-substituted or unsubstituted —(C₁₋₄alkyl)-Z-aryl. In other embodiments, R₂, and/or R₂ₐ can be a mono-substituted, poly-substituted or unsubstituted —(C₁₋₄alkyl)C(═O). In yet other embodiments, R₂, and/or R₂ₐ can be a mono-substituted, poly-substituted or unsubstituted —N(R₁)—C(=Z)R₁. In some embodiments, R₂, and/or R₂ₐ can be a mono-substituted, poly-substituted or unsubstituted —N(R₁)—C(=Z)NR₁ₐR_{1b}. In other embodiments, R₂, and/or R₂ₐ can be a mono-substituted, poly-substituted or unsubstituted —S(O)NR₁ₐR_{1b}. In yet other embodiments, R₂, and/or R₂ₐ can be a mono-substituted, poly-substituted or unsubstituted —S(O)₂NR₁ₐR_{1b}. In some embodiments, R₂, and/or R₂ₐ can be a mono-substituted, poly-substituted or unsubstituted —N(R₁)—S(═O)R₁. In other embodiments, R₂, and/or R₂ₐ can be a mono-substituted, poly-substituted or unsubstituted —N(R₁)—S(═O)₂R₁. In yet other embodiments, R₂, and/or R₂ₐ can be a mono-substituted, poly-substituted or unsubstituted —OR₁. In yet other embodiments, R₂, and/or R₂ₐ can be a mono-substituted, poly-substituted or unsubstituted —SR₁. In some embodiments, R₂, and/or R₂ₐ can be a mono-substituted, poly-substituted or unsubstituted —OC(═O)R₁. In some embodiments, R₂, and/or R₂ₐ can be a mono-substituted, poly-substituted or unsubstituted sulfinyl. In other embodiments, R₂, and/or R₂ₐ can be a mono-substituted, poly-substituted or unsubstituted sulfonyl. In yet other embodiments, R₂, and/or R₂ₐ can be a mono-substituted, poly-substituted or unsubstituted haloalkyl. In yet still other embodiments, R₂, and/or R₂ₐ can be a mono-substituted, poly-substituted or unsubstituted haloalkoxy.

In some embodiments, R₂ₐ can be selected from the group consisting mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl. aralkyl, heteroaralkyl, (heteroalicyclylalkyl, —(C₁₋₄alkyl)-Z-aryl, and —(C₁₋₄alkyl)C(═O)R₁. In one embodiment, R₂ₐ can be a mono-substituted, poly-substituted or unsubstituted alkyl. In another embodiment, R₂ₐ can be a mono-substituted, poly-substituted or unsubstituted alkenyl. In still another embodiment, R₂ₐ can be a mono-substituted, poly-substituted or unsubstituted alkynyl. In yet still another embodiment, R₂ₐ can be a mono-substituted, poly-substituted or unsubstituted cycloalkyl. In one embodiment, R₂ₐ can be a mono-substituted, poly-substituted or unsubstituted cycloalkenyl. In another embodiment, R₂ₐ can be a mono-substituted, poly-substituted or unsubstituted cycloalkynyl. In still another embodiment, R₂ₐ can be a mono-substituted, poly-substituted or unsubstituted aryl. In yet still another embodiment, R₂ₐ can be a mono-substituted, poly-substituted or unsubstituted heteroaryl. (e.g., substituted or unsubstituted pyridine). In one embodiment, R₂ₐ can be a mono-substituted, poly-substituted or unsubstituted heteroalicyclyl. In another embodiment, R₂ₐ can be a mono-substituted, poly-substituted or unsubstituted aralkyl (e.g., substituted or unsubstitued phenyl(methyl), substituted or unsubstitued phenyl(ethyl)), substituted or unsubstitued phenyl(propyl)). In still another embodiment, R₂ₐ can be a mono-substituted, poly-substituted or unsubstituted heteroaralkyl (e.g., substituted or unsubstituted indole). In yet still another embodiment, R₂ₐ can be a mono-substituted, poly-substituted or unsubstituted (heteroalicyclyl)alkyl. In some embodiments, R₂ₐ can be —(C₁₋₄alkyl)-Z-aryl. In other embodiments, R₂ₐ can be (C₁₋₄alkyl)C(—O)R₁. In any of the embodiments of described in this paragraph, R₂ can be hydrogen. In any of the embodiments of described in this paragraph, R₂ can be an alkyl such as methyl.

In certain embodiments, the cycloalkenyl can be 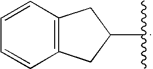
In some embodiments, R₁ and R₂ₐ cannot both be hydrogen. In other embodiments, R₂ cannot be hydrogen when R₂ₐ is an alkyl. In other embodiments, R₂ and R₂ₐ cannot both be a lower alkyl. In still other embodiments, R₂ and R₂ₐ cannot be hydrogen or alkyl when B is —C(═O) NR₁ₐR_{1b}.

In some embodiments, R₂ and R₂ₐ can be taken together, along with the nitrogen atom to which they are attached, to form an unsubstituted or substituted heteroalicyclyl. Examples wherein R₂ and R₂ₐ can be taken together, along with the nitrogen atom to which they are attached, to form an unsubstituted or substituted heteroalicyclyl include but are not limited to the following:

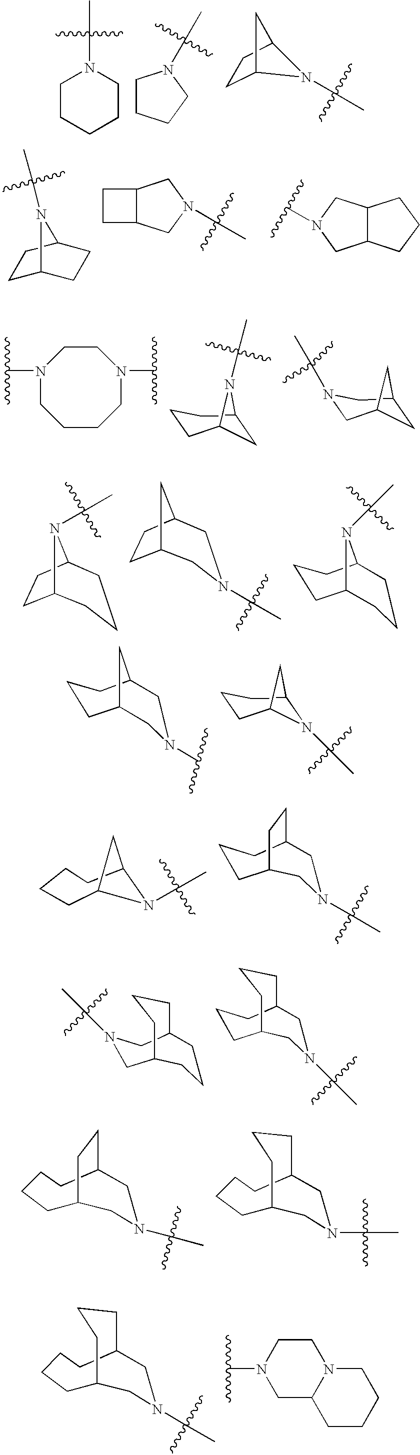

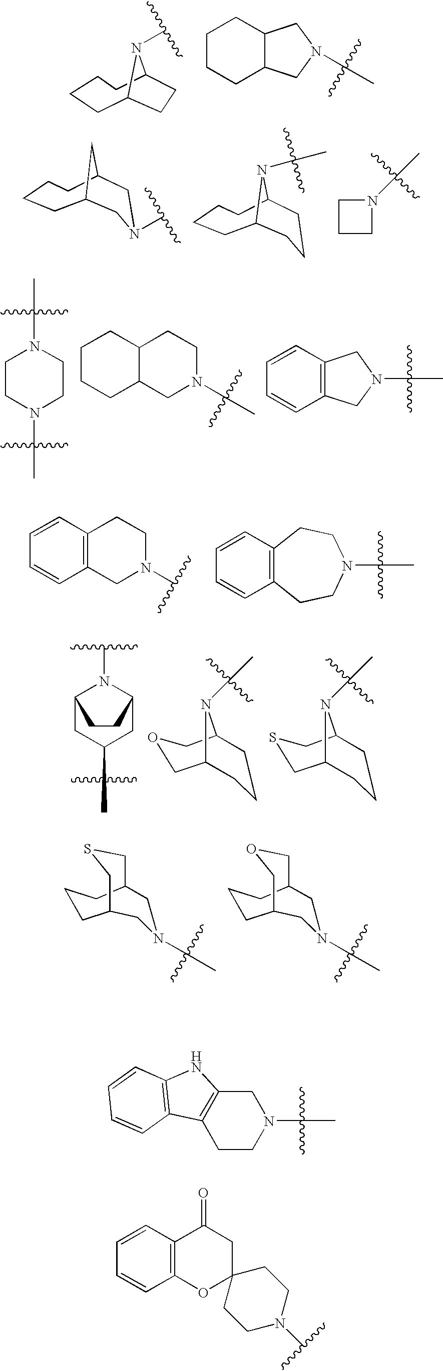

In certain embodiments, R₂ and R₂ₐ can be taken together, along with the nitrogen atom to which they are attached, to form an unsubstituted or substituted 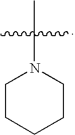
In other embodiments, R₂ and R₂ₐ can be taken together, along with the nitrogen atom to which they are attached, to form an unsubstituted or substituted 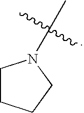
In yet other embodiments, R₂ and R₂ₐ can be taken together, along with the nitrogen atom to which they are attached, to form an unsubstituted or substituted 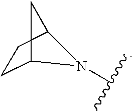
In some embodiments, R₂ and R₂ₐ can be taken together, along with the nitrogen atom to which they are attached, to form an unsubstituted or substituted 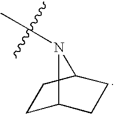
In other embodiments, R₂ and R₂ₐ can be taken together, along with the nitrogen atom to which they are attached, to form an unsubstituted or substituted 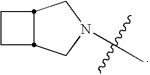
In yet other embodiments, R₂ and R₂ₐ can be taken together, along with the nitrogen atom to which they are attached, to form an unsubstituted or substituted 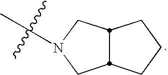
In still other embodiments, R₂ and R₂ₐ can be taken together, along with the nitrogen atom to which they are attached, to form an unsubstituted or substituted 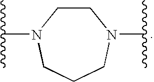
In still other embodiments, R₂ and R₂ₐ can be taken together, along with the nitrogen atom to which they are attached, to form an unsubstituted or substituted 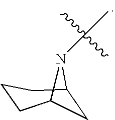
In some embodiments, R₂ and R₂ₐ can be taken together, along with the nitrogen atom to which they are attached, to form an unsubstituted or substituted

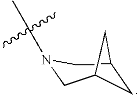

In other embodiments, R₂ and R₂ₐ can be taken together, along with the nitrogen atom to which they are attached, to form an unsubstituted or substituted 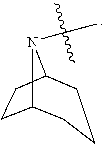
In yet other embodiments, R₂ and R₂ₐ can be taken together, along with the nitrogen atom to which they are attached, to form an unsubstituted or substituted 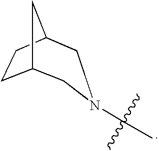
In still other embodiments, R₂ and R₂ₐ can be taken together, along with the nitrogen atom to which they are attached, to form an unsubstituted or substituted 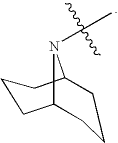
In yet other embodiments, R₂ and R₂ₐ can be taken together, along with the nitrogen atom to which they are attached, to form an unsubstituted or substituted 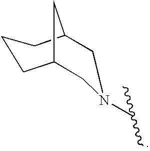
In other embodiments, R₂ and R₂ₐ can be taken together, along with the nitrogen atom to which they are attached, to form an unsubstituted or substituted 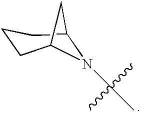
In certain embodiments, R₂ and R₂ₐ can be taken together, along with the nitrogen atom to which they are attached, to form an unsubstituted or substituted 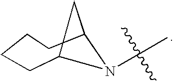
In other embodiments, R₂ and R₂ₐ can be taken together, along with the nitrogen atom to which they are attached, to form an unsubstituted or substituted 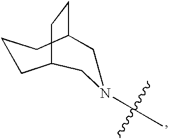
In yet other embodiments, R₂ and R₂ₐ can be taken together, along with the nitrogen atom to which they are attached, to form an unsubstituted or substituted 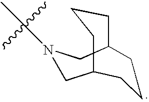
In some embodiments, R₂ and R₂ₐ can be taken together, along with the nitrogen atom to which they are attached, to form an unsubstituted or substituted 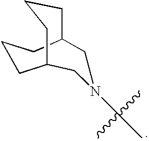
In other embodiments, R₂ and R₂ₐ can be taken together, along with the nitrogen atom to which they are attached, to form an unsubstituted or substituted 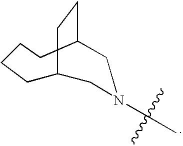
In yet other embodiments, R₂ and R₂ₐ can be taken together, along with the nitrogen atom to which they are attached, to form an unsubstituted or substituted 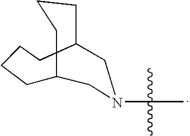
In still other embodiments, R₂ and R₂ₐ can be taken together, along with the nitrogen atom to which they are attached, to form an unsubstituted or substituted 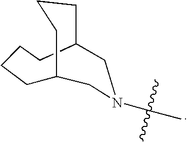
In still other embodiments, R₂ and R₂ₐ can be taken together, along with the nitrogen atom to which they are attached, to form an unsubstituted or substituted 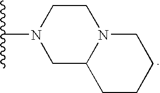
In some embodiments, R₂ and R₂ₐ can be taken together, along with the nitrogen atom to which they are attached, to form an unsubstituted or substituted 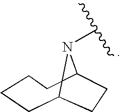
In other embodiments, R₂ and R₂ₐ can be taken together, along with the nitrogen atom to which they are attached, to form an unsubstituted or substituted 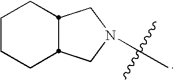
In still other embodiments, R₂ and R₂ₐ can be taken together, along with the nitrogen atom to which they are attached, to form an unsubstituted or substituted 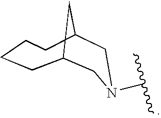
In other embodiments, R₂ and R₂ₐ can be taken together, along with the nitrogen atom to which they are attached, to form an unsubstituted or substituted 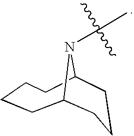
In yet other embodiments, R₂ and R₂ₐ can be taken together, along with the nitrogen atom to which they are attached, to form an unsubstituted or substituted 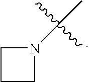
In still other embodiments, R₂ and R₂ₐ can be taken together, along with the nitrogen atom to which they are attached, to form an unsubstituted or substituted 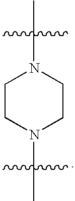
In still other embodiments, R₂ and R₂ₐ can be taken together, along with the nitrogen atom to which they are attached, to form an unsubstituted or substituted 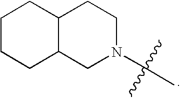
In some embodiments, R₂ and R₂ₐ can be taken together, along with the nitrogen atom to which they are attached, to form an unsubstituted or substituted 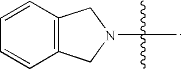
In certain embodiments, R₂ and R₂ₐ can be taken together, along with the nitrogen atom to which they are attached, to form an unsubstituted or substituted 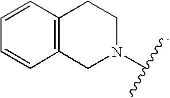
In other embodiments, R₂ and R₂ₐ can be taken together, along with the nitrogen atom to which they are attached, to form an unsubstituted or substituted 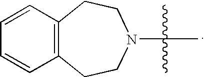
In yet other embodiments, R₂ and R₂ₐ can be taken together, along with the nitrogen atom to which they are attached, to form an unsubstituted or substituted 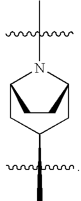
In some embodiments, R₂ and R₂ₐ can be taken together, along with the nitrogen atom to which they are attached, to form an unsubstituted or substituted 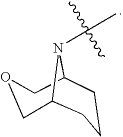
In other embodiments, R₂ and R₂ₐ can be taken together, along with the nitrogen atom to which they are attached, to form an unsubstituted or substituted 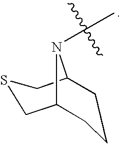
In yet other embodiments, R₂ and R₂ₐ can be taken together, along with the nitrogen atom to which they are attached, to form an unsubstituted or substituted 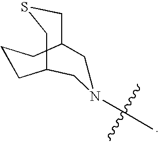
In still other embodiments, R₂ and R₂ₐ can be taken together, along with the nitrogen atom to which they are attached, to form an unsubstituted or substituted 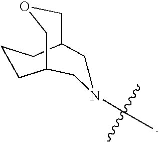
In still other embodiments, R₂ and R₂ₐ can be taken together, along with the nitrogen atom to which they are attached, to form an unsubstituted or substituted 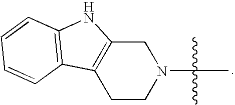
In some embodiments, R₂ and R₂ₐ can be taken together, along with the nitrogen atom to which they are attached, to form an unsubstituted or substituted

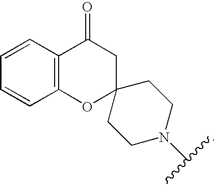

In some embodiments, R₂ and R₂ₐ can be taken together, along with the nitrogen atom to which they are attached, to form an unsubstituted or substituted heteroalicyclyl selected from the group consisting of: 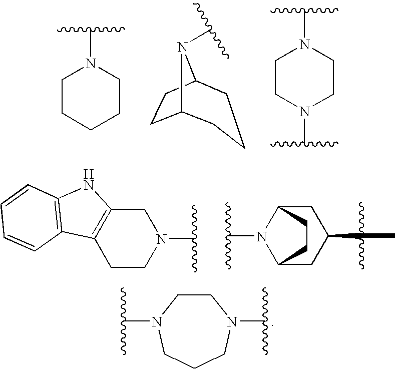
In certain embodiments, R₂ and R₂ₐ can be taken together, along with the nitrogen atom to which they are attached, to form an unsubstituted or substituted

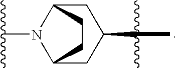

When R₂ and R₂ₐ are taken together, along with the nitrogen atom to which they are attached, to form a substituted heteroalicyclyl such as those described herein, the substituted heteroalicyclyl can be substituted with one or more group(s) individually and independently selected from the group consisting of hydrogen, halogen, cyano, nitro, hydroxyl, mono-substituted, poly-substituted or unsubstituted variants of the following residues: alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, alkoxy, aryloxy, ester, mercapto, alkylthio, arylthio, carbonyl, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, C-carboxy, O-carboxy, silyl, sulfenyl, sulfinyl, sulfonyl, haloalkyl, haloalkoxy, trihalomethanesulfonyl, trihalomethanesulfonamido, and amino.

In certain embodiments, R₂ and R₂ₐ are taken together, along with the nitrogen atom to which they are attached, to form a substituted heteroalicyclyl such as those described herein (e.g., paragraphs [0014] and [0015]), wherein the substituted heteroalicyclyl can be substituted with one or more group(s) suitable groups. In some embodiments, the substituted heteroalicyclyl is substituted with 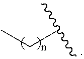
In certain embodiments, 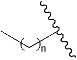
can be n-butyl or n-pentyl. In other embodiments, the substituted heteroalicyclyl can be substituted with 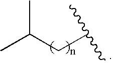
In yet other embodiments, the substituted heteroalicyclyl can be substituted with 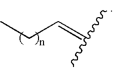
In some embodiments, the substituted heteroalicyclyl can be substituted with 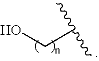
In other embodiments, the substituted heteroalicyclyl can be substituted with 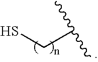
In yet other embodiments, the substituted heteroalicyclyl can be substituted with 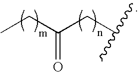
In still other embodiments, the substituted heteroalicyclyl can be substituted with 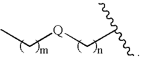
In still other embodiments, the substituted heteroalicyclyl can be substituted with 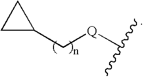
In some embodiments, the substituted heteroalicyclyl can be substituted with 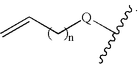
In other embodiments, the substituted heteroalicyclyl can be substituted with 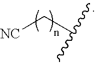
In yet other embodiments, the substituted heteroalicyclyl can be substituted with 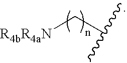
In still other embodiments, the substituted heteroalicyclyl can be substituted with 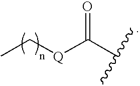
In yet other embodiments, the substituted heteroalicyclyl can be substituted with 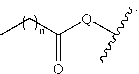
In other embodiments, the substituted heteroalicyclyl can be substituted with 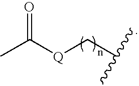
In certain embodiments, the substituted heteroalicyclyl can be substituted with 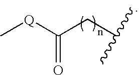
In other embodiments, the substituted heteroalicyclyl can be substituted with 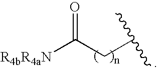
In yet other embodiments, the substituted heteroalicyclyl can be substituted with 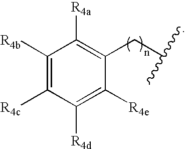
In some embodiments, the substituted heteroalicyclyl can be substituted with 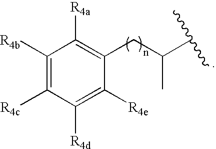
In other embodiments, the substituted heteroalicyclyl can be substituted with 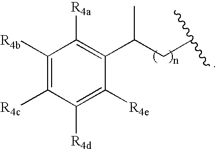
In yet other embodiments, the substituted heteroalicyclyl can be substituted with 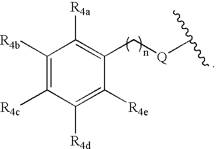
In still other embodiments, the substituted heteroalicyclyl can be substituted with

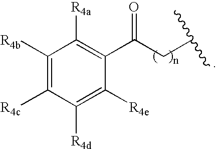

In still other embodiments, the substituted heteroalicyclyl can be substituted with 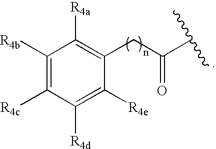
In some embodiments, the substituted heteroalicyclyl can be substituted with 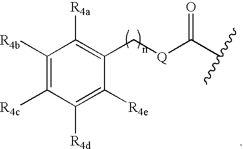
In other embodiments, the substituted heteroalicyclyl can be substituted with 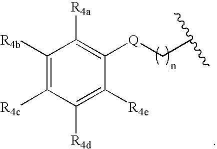
In still other embodiments, the substituted heteroalicyclyl can be substituted with 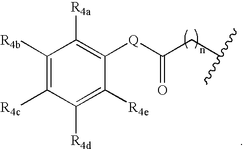
In other embodiments, the substituted heteroalicyclyl can be substituted with 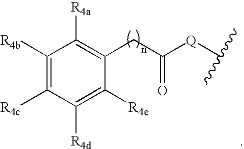
In yet other embodiments, the substituted heteroalicyclyl can be substituted with 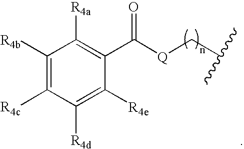
In still other embodiments, the substituted heteroalicyclyl can be substituted with 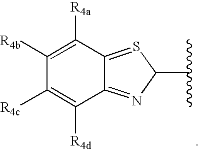
In still other embodiments, the substituted heteroalicyclyl can be substituted with 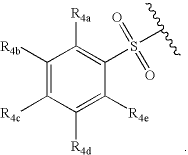
In some embodiments, the substituted heteroalicyclyl can be substituted with 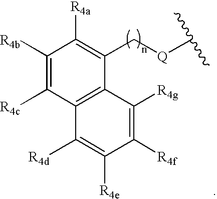
In other embodiments, the substituted heteroalicyclyl can be substituted with 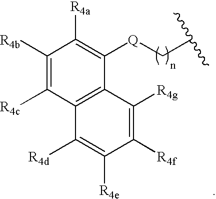
In some embodiments, the substituted heteroalicyclyl can be substituted with 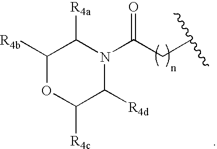
In other embodiments, the substituted heteroalicyclyl can be substituted with 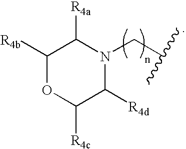
In still other embodiments, the substituted heteroalicyclyl can be substituted with 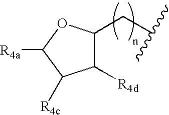
In other embodiments, the substituted heteroalicyclyl can be substituted 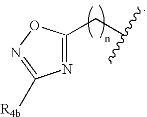
In yet other embodiments, the substituted heteroalicyclyl can be substituted with 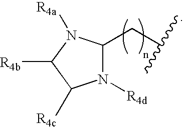
In still other embodiments, the substituted heteroalicyclyl can be substituted with 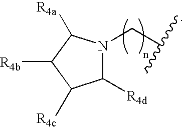
In still other embodiments, the substituted heteroalicyclyl can be substituted with 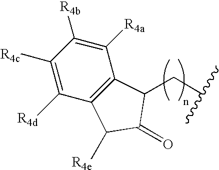
In some embodiments, the substituted heteroalicyclyl can be substituted with 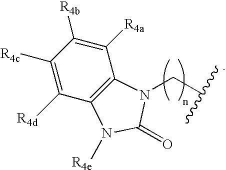
In other embodiments, the substituted heteroalicyclyl can be substituted with 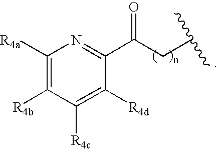
In still other embodiments, the substituted heteroalicyclyl can be substituted with 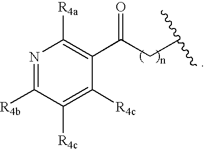
In other embodiments, the substituted heteroalicyclyl can be substituted with 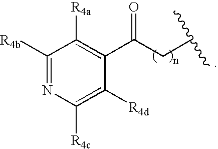
In yet other embodiments, the substituted heteroalicyclyl can be substituted with 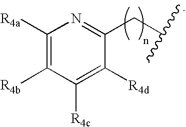
In still other embodiments, the substituted heteroalicyclyl can be substituted with 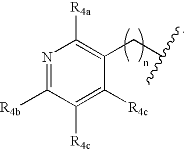
In still other embodiments, the substituted heteroalicyclyl can be substituted with 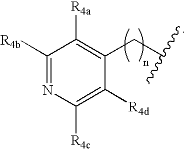
In some embodiments, the substituted heteroalicyclyl can be substituted with

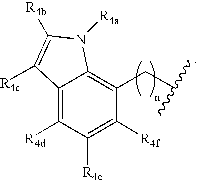

In certain embodiments, R₂ and R₂ₐ cannot be taken together, along with the nitrogen atom to which they are attached, to form an unsubstituted or substituted N-morpholinyl, an unsubstituted or substituted piperidinyl, an unsubstituted or substituted piperiazinyl, an unsubstituted or substituted azetidinyl, or an N-oxide of an amine group when B is —C(═O)R₁, wherein R₁ is an unsubstituted or substituted phenyl, an unsubstituted or substituted C₆₋₈ cycloalkyl, unsubstituted or substituted styryl, unsubstituted or substituted biphenyl, unsubstituted or substituted napthyl, unsubstituted or substituted anthryl, unsubstituted or substituted thienyl, unsubstitited furyl, unsubstituted or substituted quinolyl, or unsubstituted or substituted pyrrolyl. In some embodiments, R₂ and R₂ₐ cannot be selected from the group consisting of hydrogen, C₁₋₆alkyl, C₃₋₁₅cycloalkyl, C₃₋₁₅heterocycloalkyl, heteroaryl and aryl. In some embodiments, R₂ and R₂ₐ cannot be taken together along with the nitrogen atom to which they are attached, to form a heteroaryl.

In some of the embodiments described herein, n can be 0. In other embodiments described herein, n can be 1. In yet other embodiments described herein, n can be 2. In some embodiments described herein, n can be 3. In other embodiments described herein, n can be 4. In yet other embodiments described herein, n can be 5. In still other embodiments described herein, n can be 6.

In some of the embodiments described herein, m can be 0. In other embodiments described herein, m can be 1. In yet other embodiments described herein, m can be 2. In some of the embodiments described herein, m can be 3. In other embodiments described herein, m can be 4. In yet other embodiments described herein, m can be 5. In still other embodiments described herein, m can be 6. Also, included herein are any combination of n and m (e.g., n is 0 and m is 2, n is 3 and m is 1, n is 2 and m is 0, etc.).

In some embodiments described herein, Q can be oxygen. In other embodiments described herein Q can be sulfur.

When R₂ and R₂ₐ are taken together, along with the nitrogen atom to which they are attached, to form a substituted heteroalicyclyl, the group(s) substituents attached to the substituted heteroalicyclyl can be also be substituted. In some embodiments, any one or more of R₄ₐ, R_{4b}, R_{4c}, R_{4d}, R₄ₑ, R_{4f} and R_{4g} can be hydrogen. In other embodiments, any one or more of R₄ₐ, R_{4b}, R_{4c}, R_{4d}, R₄ₑ, R_{4f} and R_{4g} can be halogen. In yet other embodiments, any one or more of R₄ₐ, R_{4b}, R_{4c}, R_{4d}, R₄ₑ, R_{4f} and R_{4g} can be cyano. In some embodiments, any one or more of R₄ₐ, R_{4b}, R_{4c}, R_{4d}, R₄ₑ, R_{4f} and R_{4g} can be nitro. In other embodiments, any one or more of R₄ₐ, R_{4b}, R_{4c}, R_{4d}, R₄ₑ, R_{4f} and R_{4g} can be hydroxyl. In yet other embodiments, any one or more of R₄ₐ, R_{4b}, R_{4c}, R_{4d}, R₄ₑ, R_{4f} and R_{4g} can be a mono-substituted, poly-substituted or unsubstituted alkyl. In still other embodiments, any one or more of R₄ₐ, R_{4b}, R_{4c}, R_{4d}, R₄ₑ, R_{4f} and R_{4g} can be a mono-substituted, poly-substituted or unsubstituted alkenyl. In still other embodiments, any one or more of R₄ₐ, R_{4b}, R_{4c}, R_{4d}, R₄ₑ, R_{4f} and R_{4g} can be a mono-substituted, poly-substituted or unsubstituted alkynyl. In some embodiments, any one or more of R₄ₐ, R_{4b}, R_{4c}, R_{4d}, R₄ₑ, R_{4f} and R_{4g} can be a mono-substituted, poly-substituted or unsubstituted cycloalkyl. In other embodiments, any one or more of R₄ₐ, R_{4b}, R_{4c}, R_{4d}, R₄ₑ, R_{4f} and R_{4g} can be a mono-substituted, poly-substituted or unsubstituted cycloalkenyl. In yet other embodiments, any one or more of R₄ₐ, R_{4b}, R_{4c}, R_{4d}, R₄ₑ, R_{4f} and R_{4g} can be a mono-substituted, poly-substituted or unsubstituted cycloalkynyl. In still other embodiments, any one or more of R₄ₐ, R_{4b}, R_{4c}, R_{4d}, R₄ₑ, R_{4f} and R_{4g} can be a mono-substituted, poly-substituted or unsubstituted aryl. In yet other embodiments, any one or more of R₄ₐ, R_{4b}, R_{4c}, R_{4d}, R₄ₑ, R_{4f} and R_{4g} can be a mono-substituted, poly-substituted or unsubstituted heteroaryl. In other embodiments, any one or more of R₄ₐ, R_{4b}, R_{4c}, R_{4d}, R₄ₑ, R_{4f} and R_{4g} can be a mono-substituted, poly-substituted or unsubstituted aralkyl. In certain embodiments, any one or more of R₄ₐ, R_{4b}, R_{4c}, R_{4d}, R₄ₑ, R_{4f} and R_{4g} can be a mono-substituted, poly-substituted or unsubstituted heteroaralkyl. In other embodiments, any one or more of R₄ₐ, R_{4b}, R_{4c}, R_{4d}, R₄ₑ, R_{4f} and R_{4g} can be a mono-substituted, poly-substituted or unsubstituted heteroalicyclyl. In yet other embodiments, any one or more of R₄ₐ, R_{4b}, R_{4c}, R_{4d}, R₄ₑ, R_{4f} and R_{4g} can be a mono-substituted, poly-substituted or unsubstituted (heteroalicyclyl)alkyl. In some embodiments, any one or more of R₄ₐ, R_{4b}, R_{4c}, R_{4d}, R₄ₑ, R_{4f} and R_{4g} can be a mono-substituted, poly-substituted or unsubstituted alkoxy. In other embodiments, any one or more of R₄ₐ, R_{4b}, R_{4c}, R_{4d}, R₄ₑ, R_{4f} and R_{4g} can be a mono-substituted, poly-substituted or unsubstituted aryloxy. In yet other embodiments, any one or more of R₄ₐ, R_{4b}, R_{4c}, R_{4d}, R₄ₑ, R_{4f} and R_{4g} can be a mono-substituted, poly-substituted or unsubstituted ester. In certain embodiments, any one or more of R₄ₐ, R_{4b}, R_{4c}, R_{4d}, R₄ₑ, R_{4f} and R_{4g} can be a mono-substituted, poly-substituted or unsubstituted mercapto. In other embodiments, any one or more of R₄ₐ, R_{4b}, R_{4c}, R_{4d}, R₄ₑ, R_{4f} and R_{4g} can be a mono-substituted, poly-substituted or unsubstituted alkylthio. In yet other embodiments, any one or more of R₄ₐ, R_{4b}, R_{4c}, R_{4d}, R₄ₑ, R_{4f} and R_{4g} can be a mono-substituted, poly-substituted or unsubstituted arylthio. In some embodiments, any one or more of R₄ₐ, R_{4b}, R_{4c}, R_{4d}, R₄ₑ, R_{4f} and R_{4g} can be a mono-substituted, poly-substituted or unsubstituted carbonyl. In other embodiments, any one or more of R₄ₐ, R_{4b}, R_{4c}, R_{4d}, R₄ₑ, R_{4f} and R_{4g} can be a mono-substituted, poly-substituted or unsubstituted thiocarbonyl. In yet other embodiments, any one or more of R₄ₐ, R_{4b}, R_{4c}, R_{4d}, R₄ₑ, R_{4f} and R_{4g} can be a mono-substituted, poly-substituted or unsubstituted O-carbamyl. In some embodiments, any one or more of R₄ₐ, R_{4b}, R_{4c}, R_{4d}, R₄ₑ, R_{4f} and R_{4g} can be a mono-substituted, poly-substituted or unsubstituted N-carbamyl. In other embodiments, any one or more of R₄ₐ, R_{4b}, R_{4c}, R_{4d}, R₄ₑ, R_{4f} and R_{4g} can be a mono-substituted, poly-substituted or unsubstituted O-thiocarbamyl. In yet other embodiments, any one or more of R₄ₐ, R_{4b}, R_{4c}, R_{4d}, R₄ₑ, R_{4f} and R_{4g} can be a mono-substituted, poly-substituted or unsubstituted N-thiocarbamyl. In some embodiments, any one or more of R₄ₐ, R_{4b}, R_{4c}, R_{4d}, R₄ₑ, R_{4f} and R_{4g} can be a mono-substituted, poly-substituted or unsubstituted C-amido. In other embodiments, any one or more of R₄ₐ, R_{4b}, R_{4c}, R_{4d}, R₄ₑ, R_{4f} and R_{4g} can be a mono-substituted, poly-substituted or unsubstituted N-amido. In yet other embodiments, any one or more of R₄ₐ, R_{4b}, R_{4c}, R_{4d}, R₄ₑ, R_{4f} and R_{4g} can be a mono-substituted, poly-substituted or unsubstituted S-sulfonamido. In certain embodiments, any one or more of R₄ₐ, R_{4b}, R_{4c}, R_{4d}, R₄ₑ, R_{4f} and R_{4g} can be a mono-substituted, poly-substituted or unsubstituted N-carbamyl. In other embodiments, any one or more of R₄ₐ, R_{4b}, R_{4c}, R_{4d}, R₄ₑ, R_{4f} and R_{4g} can be a mono-substituted, poly-substituted or unsubstituted N-sulfonamido. In yet other embodiments, any one or more of R₄ₐ, R_{4b}, R_{4c}, R_{4d}, R₄ₑ, R_{4f} and R_{4g} can be a mono-substituted, poly-substituted or unsubstituted isocyanato. In some embodiments, any one or more of R₄ₐ, R_{4b}, R_{4c}, R_{4d}, R₄ₑ, R_{4f} and R_{4g} can be a mono-substituted, poly-substituted or unsubstituted thiocyanato. In other embodiments any one or more of R₄ₐ, R_{4b}, R_{4c}, R_{4d}, R₄ₑ, R_{4f} and R_{4g} can be a mono-substituted, poly-substituted or unsubstituted isothiocyanato. In yet other embodiments, any one or more of R₄ₐ, R_{4b}, R_{4c}, R_{4d}, R₄ₑ, R_{4f} and R_{4g} can be a mono-substituted, poly-substituted or unsubstituted C-carboxy. In certain embodiments, any one or more of R₄ₐ, R_{4b}, R_{4c}, R_{4d}, R₄ₑ, R_{4f} and R_{4g} can be a mono-substituted, poly-substituted or unsubstituted O-carboxy. In other embodiments, any one or more of R₄ₐ, R_{4b}, R_{4c}, R_{4d}, R₄ₑ, R_{4f} and R_{4g} can be a mono-substituted, poly-substituted or unsubstituted silyl. In yet other embodiments, any one or more of R₄ₐ, R_{4b}, R_{4c}, R_{4d}, Rₑ, R_{4f} and R_{4g} can be a mono-substituted, poly-substituted or unsubstituted sulfenyl. In some embodiments, any one or more of R₄ₐ, R_{4b}, R_{4c}, R_{4d}, R₄ₑ, R_{4f} and R_{4g} can be a mono-substituted, poly-substituted or unsubstituted sulfinyl. In other embodiments, any one or more of R₄ₐ, R_{4b}, R_{4c}, R_{4d}, R₄ₑ, R_{4f} and R_{4g} can be a mono-substituted, poly-substituted or unsubstituted sulfonyl. In yet other embodiments, any one or more of R₄ₐ, R_{4b}, R_{4c}, R_{4d}, R₄ₑ, R_{4f} and R_{4g} can be a mono-substituted, poly-substituted or unsubstituted haloalkyl. In certain embodiments, any one or more of R₄ₐ, R_{4b}, R_{4c}, R_{4d}, R₄ₑ, R_{4f} and R_{4g} can be a mono-substituted, poly-substituted or unsubstituted haloalkoxy. In other embodiments, any one or more of R₄ₐ, R_{4b}, R_{4c}, R_{4d}, R₄ₑ, R_{4f} and R_{4g} can be a mono-substituted, poly-substituted or unsubstituted trihalomethanesulfonyl, In yet other embodiments, any one or more of R₄ₐ, R_{4b}, R_{4c}, R_{4d}, R₄ₑ, R_{4f} and R_{4g} can be a mono-substituted, poly-substituted or unsubstituted trihalomethanesulfonamido. In some embodiments, any one or more of R₄ₐ, R_{4b}, R_{4c}, R_{4d}, R₄ₑ, R_{4f} and R_{4g} can be a mono-substituted, poly-substituted or unsubstituted amino. Also, include herein are any combination of any one or more of R₄ₐ, R_{4b}, R_{4c}, R_{4d}, R₄ₑ, R_{4f} and R_{4g} as described in this paragraph (e.g., R₄ₐ is H and R_{4d} is halogen, R_{4d} is alkyl and R_{4b} is haloalkyl, etc.).

In certain embodiments, R₂ and R₂ₐ can be taken together, along with the nitrogen atom to which they are attached, to form an unsubstituted or substituted heteroalicyclyl, which is unsubstituted or substituted with one or more group(s) individually and independently selected from the group consisting of:

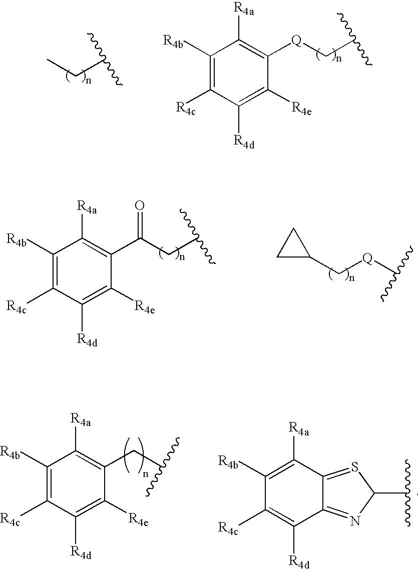

In some embodiments, any one or more of R_{1b} R₄ₐ, R_{4b}, R_{4c}, R_{4d}, R₄ₑ, R_{4f} and R_{4g} can each independently be selected from the group consisting of hydrogen, halogen, cyano, nitro, hydroxyl, mono-substituted, poly-substituted or unsubstituted variants of the following residues: alkyl, alkoxy, aryl, alkylthio (e.g., H₃CS—), and haloalkyl. In one embodiment, at least one of R₄ₐ, R_{4b}, R_{4c}, R_{4d}, R₄ₑ, R_{f} and/or R_{g} can be halogen. In another embodiment, more than one of R₄ₐ, R_{4b}, R_{4c}, R_{4d}, R₄ₑ, R_{f} and/or R_{g} can be halogen. In another embodiment, at least one of R₄ₐ, R_{4b}, R_{4c}, R_{4d}, R₄ₑ, R_{f} and/or R_{g} can be an alkoxy such as methoxy, ethoxy, n-propoxy, isopropoxy, n-butoxy, isobutoxy, and/or t-butoxy. Preferably, the alkoxy is methoxy. In still another embodiment at least one of R₄ₐ, R_{4b}, R_{4c}, R_{4d}, R₄ₑ, R_{f} and/or R_{g} can be an alkyl. Exemplary alkyls include but are not limited to methyl, ethyl, n-propyl, isopropyl, n-butyl, isobutyl, and t-butyl. In preferred embodiments, the alkyl can be methyl and/or ethyl. In yet still another embodiment, at least one of R₄ₐ, R_{4b}, R_{4c}, R_{4d}, R₄ₑ, R_{f} and/or R_{g} can be an aryl (e.g., pyridine). In one embodiment, at least one of R₄ₐ, R_{4b}, R_{4c}, R_{4d}, R₄ₑ, R_{f} and/or R_{g} can be a haloalkyl such as CF₃.

As to B, in some embodiments, B can be hydrogen. In other embodiments, B can be a mono-substituted, poly-substituted or unsubstituted alkyl. In yet other embodiments, B can be a mono-substituted, poly-substituted or unsubstituted alkenyl. In some embodiments, B can be a mono-substituted, poly-substituted or unsubstituted alkynyl. In other embodiments, B can be a mono-substituted, poly-substituted or unsubstituted cycloalkyl. In yet other embodiments, B can be a mono-substituted, poly-substituted or unsubstituted cycloalkenyl. In still other embodiments, B can be a mono-substituted, poly-substituted or unsubstituted cycloalkynyl. In still other embodiments, B can be a mono-substituted, poly-substituted or unsubstituted aryl. In some embodiments, B can be a mono-substituted, poly-substituted or unsubstituted heteroaryl. In other embodiments, B can be a mono-substituted, poly-substituted or unsubstituted heteroalicyclyl. In yet other embodiments, B can be a mono-substituted, poly-substituted or unsubstituted aralkyl. In still other embodiments, B can be a mono-substituted, poly-substituted or unsubstituted heteroaralkyl. In yet other embodiments, B can be a mono-substituted, poly-substituted or unsubstituted (heteroalicyclyl)alkyl. In other embodiments, B can be a mono-substituted, poly-substituted or unsubstituted —C(=Z)R₁. In some embodiments, B can be a mono-substituted, poly-substituted or unsubstituted —C(=Z)OR₁. In other embodiments, B can be a mono-substituted, poly-substituted or unsubstituted —C(Z)NR₁ₐR_{1b}. In yet other embodiments, B can be a mono-substituted, poly-substituted or unsubstituted —C(=Z)N(OR₁ₐ)R_{1b}. In some embodiments, B can be a mono-substituted, poly-substituted or unsubstituted —C(Z)N(R₁)NR₁ₐR_{1b}. In other embodiments, B can be a mono-substituted, poly-substituted or unsubstituted —C(R₁)═NR₁ₐ. In yet other embodiments, B can be a mono-substituted, poly-substituted or unsubstituted —C≡N. In some embodiments, B can be a mono-substituted, poly-substituted or unsubstituted —NR₁ₐR_{1b}. In other embodiments, B can be a mono-substituted, poly-substituted or unsubstituted —N═CR₁ₐR_{1b}. In yet other embodiments, B can be a mono-substituted, poly-substituted or unsubstituted —N(R₁)—C(=Z)R₁. In some embodiments, B can be a mono-substituted, poly-substituted or unsubstituted —N(R₁)—C(=Z)NR₁ₐR_{1b}. In other embodiments, B can be a mono-substituted, poly-substituted or unsubstituted —S(O)NR₁ₐR_{1b}. In yet other embodiments, B can be a mono-substituted, poly-substituted or unsubstituted —S(O)₂NR₁ₐR_{1b}. In some embodiments, B can be a mono-substituted, poly-substituted or unsubstituted, —N(R₁)—S(═O)R₁. In other embodiments, B can be a mono-substituted, poly-substituted or unsubstituted —N(R₁)—S(═O)₂R₁. In yet other embodiments, B can be a mono-substituted, poly-substituted or unsubstituted —S(O)R₁. In some embodiments, B can be a mono-substituted, poly-substituted or unsubstituted —S(O)₂R₁. In other embodiments, B can be a mono-substituted, poly-substituted or unsubstituted —OR₁. In yet other embodiments, B can be a mono-substituted, poly-substituted or unsubstituted —SR₁. In some embodiments, B can be a mono-substituted, poly-substituted or unsubstituted, —OC(═O)R₁.

In some embodiments, any one or more of R₁, R₁ₐ and R_{1b} can be hydrogen. In other embodiments, any one or more of R₁, R₁ₐ and R_{1b} can be a mono-substituted, poly-substituted or unsubstituted alkyl. In certain embodiment, the alkyl can be methyl, ethyl, n-propyl, isopropyl, n-butyl, isobutyl, t-butyl, linear or branched pentyl, linear or branched hexyl, linear or branched heptyl, and/or linear or branched octyl. Preferably, the alkyl can be methyl, ethyl, n-butyl, isobutyl, linear hexyl, and/or branched octyl. In yet other embodiments, any one or more of R₁, R₁ₐ and R_{1b} can be a mono-substituted, poly-substituted or unsubstituted alkenyl. In some embodiments, any one or more of R₁, R₁ₐ and R_{1b} can be a mono-substituted, poly-substituted or unsubstituted alkynyl. In other embodiments, any one or more of R₁, R₁ₐ and R_{1b} can be a mono-substituted, poly-substituted or unsubstituted cycloalkyl. In a preferred embodiment, the cycloalkyl is cyclopropyl. In yet other embodiments, any one or more of R₁, R₁ₐ and R_{1b} can be a mono-substituted, poly-substituted or unsubstituted cycloalkenyl. In still other embodiments, any one or more of R₁, R₁ₐ and R_{1b} can be a mono-substituted, poly-substituted or unsubstituted cycloalkynyl. In still other embodiments, any one or more of R₁, R₁ₐ and R_{1b} can be a mono-substituted, poly-substituted or unsubstituted aryl such as phenyl. In some embodiments, any one or more of R₁, R₁ₐ and R_{1b} can be a mono-substituted, poly-substituted or unsubstituted heteroaryl. In other embodiments, any one or more of R₁, R₁ₐ and R_{1b} can be a mono-substituted, poly-substituted or unsubstituted heteroalicyclyl. In yet other embodiments, any one or more of R₁, R₁ₐ and R_{1b} can be a mono-substituted, poly-substituted or unsubstituted aralkyl. In certain embodiment, the aralkyl is an optionally substituted phenyl(C₁₋₄alkyl) such as optionally substituted phenyl(methyl). If substituted, the phenyl(C₁₋₄alkyl) can be substituted with one or more substituents including but not limited to alkyl (e.g., methyl) and/or halogen. In still other embodiments, any one or more of R₁, R₁ₐ and R_{1b} can be a mono-substituted, poly-substituted or unsubstituted heteroaralkyl. In yet other embodiments, any one or more of R₁, R₁ₐ and R_{1b} can be a mono-substituted, poly-substituted or unsubstituted (heteroalicyclyl)alkyl. In other embodiments, any one or more of R₁, R₁ₐ and R_{1b} can be a haloalkyl, for example CF₃.

In some embodiments when B is —C(═O)R₁, R₁, can be a mono-substituted, poly-substituted or unsubstituted variant of the following residues: alkyl, alkenyl, cycloalkyl, aryl, aralkyl, and/or haloalkyl. In other embodiments when B is —C(═O)R₁, R₁, can be a mono-substituted, poly-substituted or unsubstituted alkyl. In still other embodiments when B is —C(═O)R₁, R₁, can be a mono-substituted, poly-substituted or unsubstituted alkenyl. In yet still other embodiments when B is —C(═O)R₁, R₁, can be a mono-substituted, poly-substituted or unsubstituted cycloalkyl. In some embodiments when B is —C(═O)R₁, R₁, can be a mono-substituted, poly-substituted or unsubstituted aryl. In other embodiments when B is —C(═O)R₁, R₁, can be a mono-substituted, poly-substituted or unsubstituted aralkyl. In still other embodiments when B is —C(—O)R₁, R₁, can be a mono-substituted, poly-substituted or unsubstituted haloalkyl.

In some embodiments, when B is —C(═O)NR₁ₐNR_{1b}, R₁ₐ and/or R_{1b} can each be a mono-substituted, poly-substituted or unsubstituted variant of the following residues: alkyl, alkenyl, cycloalkyl, aryl, aralkyl, and/or haloalkyl. In other embodiments when B is —C(═O)NR₁ₐNR_{1b}, R₁ₐ and/or R_{1b} can each be a mono-substituted, poly-substituted or unsubstituted alkyl. In still other embodiments when B is C(═O)NR₁ₐNR_{1b}, R₁ₐ and/or R_{1b} can each be a mono-substituted, poly-substituted or unsubstituted alkenyl. In yet still other embodiments when B is —C(═O)NR₁ₐNR_{1b}, R₁ₐ and/or R_{1b} can each be a mono-substituted, poly-substituted or unsubstituted cycloalkyl. In some embodiments when B is —C(═O)NR₁ₐNR_{1b}, R₁ₐ and/or R_{1b} can each be a mono-substituted, poly-substituted or unsubstituted aryl. In other embodiments when B is —C(═O)NR₁ₐNR_{1b}, R₁ₐ and/or R_{1b} can each be a mono-substituted, poly-substituted or unsubstituted aralkyl. In still other embodiments when B is —C(═O)NR₁ₐNR_{1b}, R₁ₐ and/or R_{1b} can each be a mono-substituted, poly-substituted or unsubstituted haloalkyl.

In some embodiments, when B is —C(═O)OR₁, R₁, can be a mono-substituted, poly-substituted or unsubstituted variant of the following residues: alkyl, alkenyl, cycloalkyl, aryl, aralkyl, and/or haloalkyl. In other embodiments when B is —C(═O)OR₁, R₁, can be a mono-substituted, poly-substituted or unsubstituted alkyl. In still other embodiments when B is —C(—O)OR₁, R₁, can be a mono-substituted, poly-substituted or unsubstituted alkenyl. In yet still other embodiments when B is —C(═O)OR₁, R₁, can be a mono-substituted, poly-substituted or unsubstituted cycloalkyl. In some embodiments when B is —C(═O)OR₁, R₁, can be a mono-substituted, poly-substituted or unsubstituted aryl. In other embodiments when B is —C(═O)OR₁, R₁, can be a mono-substituted, poly-substituted or unsubstituted aralkyl. In still other embodiments when B is —C(═O)OR₁, R₁, can be a mono-substituted, poly-substituted or unsubstituted haloalkyl.

In some embodiments when B is —C(═O)R₁, —C(═O)NR₁ₐNR_{1b}, or —C(═O)OR₁, R₁, R₁ₐ and/or R_{1b} can be an alkyl such as methyl, ethyl, n-propyl, isopropyl, n-butyl, isobutyl, t-butyl, linear or branched pentyl, linear or branched hexyl, linear or branched heptyl, and/or linear or branched octyl. Preferably, the alkyl can be methyl, ethyl, n-butyl, isobutyl, linear hexyl, and/or branched octyl. In other embodiments when B is —C(═O)R₁, —C(═O)NR₁ₐNR_{1b}, or —C(═O)OR₁, R₁, R₁ₐ and/or R_{1b} can be a cycloalkyl such as cyclopropyl. In still other embodiments when B is —C(═O)R₁, —C(═O)NR₁ₐNR_{1b}, or —C(═O)OR₁, R₁, R₁ₐ and/or R_{1b} can be an aralkyl. In certain embodiment, the aralkyl is an optionally substituted phenyl(C₁₋₄alkyl) such as optionally substituted phenyl(methyl). If substituted, the phenyl(C₁₋₄alkyl) can be substituted with one or more substituents including but not limited to alkyl (e.g., methyl) and/or halogen. In yet still other embodiments when B is —C(═O)R₁, —C(═O)NR₁ₐNR_{1b}, or —C(═O)OR₁, R₁, R₁ₐ and/or R_{1b} can be a haloalkyl (e.g., CF₃).

With respect to R₃, R₃ₐ, R_{3b}, and R_{3c}, in some embodiments, any one or more of R₃, R₃ₐ, R_{3b}, and R_{3c} can be hydrogen. In other embodiments, any one or more of R₃, R₃ₐ, R_{3b}, and R_{3c} can be halogen. In still other embodiments, any one or more of R₃, R₃ₐ, R_{3b}, and R_{3c} can be cyano. In yet other embodiments, any one or more of R₃, R₃ₐ, R_{3b}, and R_{3c} can be nitro. In other embodiments, any one or more of R₃, R₃ₐ, R_{3b}, and R_{3c} can be a mono-substituted, poly-substituted or unsubstituted alkyl. In yet other embodiments, any one or more of R₃, R₃ₐ, R_{3b}, and R_{3c} can be a mono-substituted, poly-substituted or unsubstituted alkenyl. In some embodiments, any one or more of R₃, R₃ₐ, R_{3b}, and R_{3c} can be a mono-substituted, poly-substituted or unsubstituted alkynyl. In other embodiments, any one or more of R₃, R₃ₐ, R_{3b}, and R_{3c} can be a mono-substituted, poly-substituted or unsubstituted cycloalkyl. In yet other embodiments, any one or more of R₃, R₃ₐ, R_{3b}, and R_{3c} can be a mono-substituted, poly-substituted or unsubstituted cycloalkenyl. In still other embodiments, any one or more of R₃, R₃ₐ, R_{3b}, and R_{3c} can be a mono-substituted, poly-substituted or unsubstituted cycloalkynyl. In still other embodiments, any one or more of R₃, R₃ₐ, R_{3b}, and R_{3c} can be a mono-substituted, poly-substituted or unsubstituted aryl. In some embodiments, any one or more of R₃, R₃ₐ, R_{3b}, and R_{3c} can be a mono-substituted, poly-substituted or unsubstituted heteroaryl. In other embodiments, any one or more of R₃, R₃ₐ, R_{3b}, and R_{3c} can be a mono-substituted, poly-substituted or unsubstituted heteroalicyclyl. In yet other embodiments, any one or more of R₃, R₃ₐ, R_{3b}, and R_{3c} can be a mono-substituted, poly-substituted or unsubstituted aralkyl. In still other embodiments, any one or more of R₃, R₃ₐ, R_{3b}, and R_{3c} can be a mono-substituted, poly-substituted or unsubstituted heteroaralkyl. In yet other embodiments, any one or more of R₃, R₃ₐ, R_{3b} and R_{3c} can be a mono-substituted, poly-substituted or unsubstituted (heteroalicyclyl)alkyl. In other embodiments, any one or more of R₃, R₃ₐ, R_{3b}, and R_{3c} can be a mono-substituted, poly-substituted or unsubstituted —C(=Z)R₁. In some embodiments, any one or more of R₃, R₃ₐ, R_{3b}, and R_{3c} can be a mono-substituted, poly-substituted or unsubstituted —C(=Z)OR₁. In other embodiments, any one or more of R₃, R₃ₐ, R_{3b}, and R_{3c} can be a mono-substituted, poly-substituted or unsubstituted-C(=Z)NR₁ₐR_{1b}. In some embodiments, any one or more of R₃, R₃ₐ, R_{3b}, and R_{3c} can be a mono-substituted, poly-substituted or unsubstituted —C(R₁)═NR₁ₐ. In other embodiments, any one or more of R₃, R₃ₐ, R_{3b} and R_{3c} can be a mono-substituted, poly-substituted or unsubstituted-NR₁ₐR_{1b}. In still other embodiments, any one or more of R₃, R₃ₐ, R_{3b}, and R_{3c} can be a mono-substituted, poly-substituted or unsubstituted —N═CR₁ₐR_{1b}. In yet other embodiments, any one or more of R₃, R₃ₐ, R_{3b}, and R_{3c} can be a mono-substituted, poly-substituted or unsubstituted —N(R₁)—C(=Z)R₁. In some embodiments, any one or more of R₃, R₃ₐ, R_{3b}, and/or R_{3c} can be a mono-substituted, poly-substituted or unsubstituted —N(R₁)—C(=Z)NR₁ₐR_{1b}. In other embodiments, any one or more of R₃, R₃ₐ, R_{3b}, and R_{3c} can be a mono-substituted, poly-substituted or unsubstituted —S(O)NR₁ₐR_{1b}. In yet other embodiments, any one or more of R₃, R₃ₐ, R_{3b}, and R_{3c} can be a mono-substituted, poly-substituted or unsubstituted —S(O)₂NR₁ₐR_{1b}. In some embodiments, any one or more of R₃, R₃ₐ, R_{3b}, and R_{3c} can be a mono-substituted, poly-substituted or unsubstituted —N(R₁)—S(═O)R₁. In other embodiments, any one or more of R₃, R₃ₐ, R_{3b}, and R_{3c} can be a mono-substituted, poly-substituted or unsubstituted —N(R₁)—S(═O)₂R₁. In yet other embodiments, any one or more of R₃, R₃ₐ, R_{3b}, and R_{3c} can be a mono-substituted, poly-substituted or unsubstituted —OR₁. In yet other embodiments, any one or more of R₃, R₃ₐ, R_{3b}, and R_{3c} can be a mono-substituted, poly-substituted or unsubstituted —SR₁. In some embodiments, any one or more of R₃, R₃ₐ, R_{3b}, and R_{3c} can be a mono-substituted, poly-substituted or unsubstituted —OC(═O)R₁. In some embodiments, R_{3c} can be taken together with B to form a cycloalkyl, cycloalkenyl, cycloalkynyl, or heteroalicyclyl ring. In other embodiments, R_{3c} cannot be taken together with B to form a cycloalkyl, cycloalkenyl, cycloalkynyl, or heteroalicyclyl ring. In some embodiments, any one or more of R₃, R₃ₐ, R_{3b}, and R_{3c} can be a mono-substituted, poly-substituted or unsubstituted sulfinyl. In other embodiments, any one or more of R₃, R₃ₐ, R_{3b}, and R_{3c} can be a mono-substituted, poly-substituted or unsubstituted sulfonyl. In yet other embodiments, any one or more of R₃, R₃ₐ, R_{3b}, and R_{3c} can be a mono-substituted, poly-substituted or unsubstituted haloalkyl. In yet still other embodiments, any one or more of R₃, R₃ₐ, R_{3b}, and R_{3c} can be a mono-substituted, poly-substituted or unsubstituted haloalkoxy. In certain embodiment, any one or more of R₃, R₃ₐ, R_{3b}, and R_{3c} can be an alkyl such as methyl, ethyl, n-propyl, isopropyl, n-butyl, isobutyl, and t-butyl. Preferably, any one or more of R₃, R₃ₐ, R_{3b}, and R_{3c} can be methyl and/or ethyl. In certain embodiment, any one or more of R₃, R₃ₐ, R_{3b}, and R_{3c} can be —OR₁, wherein R₁ can be selected from the group consisting of methyl, ethyl, n-propyl, isopropyl, n-butyl, isobutyl, and t-butyl. In a preferred embodiment, R₁, can be selected from the group consisting of methyl and isopropyl.

In some embodiments, Y can be C—R₃, wherein R₃ can be selected from the group consisting of hydrogen, halogen, cyano, nitro, mono-substituted, poly-substituted or unsubstituted variants of the following residues: alkyl, alkenyl, alkynyl, cycloalkyl, cycloalkenyl cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁. In certain embodiments, Y can be C—R₃, wherein R₃ can be selected from the group consisting of alkyl, alkoxy, —C≡N, and halogen. In some embodiments, Y can be C—R₃, wherein R₃ can be an alkyl such as methyl, ethyl, n-propyl, isopropyl, n-butyl, isobutyl, and t-butyl. In an embodiment, the alkyl can be methyl or ethyl. In some embodiments, Y can be C—R₃, wherein R₃ can be an alkoxy such as methoxy, ethoxy, n-propoxy, isopropoxy, n-butoxy, isobutoxy, and t-butoxy. In other embodiments when R₃ is an alkoxy, the alkoxy can be methoxy. In some embodiments, Y can be C—R₃, wherein R₃ can be a —C≡N. In some embodiments, Y can be C—R₃, wherein R₃ can be a halogen. In some embodiments, Y can be C—R₃, wherein R₃ can be selected from the group consisting of alkyl, alkoxy, —C≡N, and halogen; and B can be —C(═O)R₁. In other embodiments, Y can be C—R₃, wherein R₃ can be selected from the group consisting of alkyl, alkoxy, —C≡N, and halogen; and B can be —C(═O)OR₁. In still other embodiments, Y can be C—R₃, wherein R₃ can be selected from the group consisting of alkyl, alkoxy, —C≡N, and halogen; and B can be —C(=Z)NR₁ₐR_{1b}. In yet still other embodiments, Y can be C—R₃, wherein R₃ can be selected from the group consisting of alkyl, alkoxy, —C≡N, and halogen; and B can be —C(=Z)N(OR₁ₐ)R_{1b}. In some embodiments, Y can be C—R₃, wherein R₃ can be selected from the group consisting of alkyl, alkoxy, —C≡N, and halogen; and B can be —C≡N. In some embodiments, Y can be C—R₃, wherein R₃ can be selected from the group consisting of alkyl (e.g., methyl), alkoxy (e.g., methoxy), —C≡N, and halogen; B can be —C(═O)R₁; and R₁ can be a mono-substituted, poly-substituted or unsubstituted alkyl such as methyl. In other embodiments, Y can be C—R₃, wherein R₃ can be selected from the group consisting of alkyl (e.g., methyl), alkoxy (e.g., methoxy), —C≡N, and halogen; B can be —C(═O)R₁; and R₁ can be a mono-substituted, poly-substituted or unsubstituted cycloalkyl such as cyclopropyl. In some embodiments, Y can be C—R₃, wherein R₃ can be selected from the group consisting of alkyl (e.g., methyl), alkoxy (e.g., methoxy), —C≡N, and halogen; B can be —C(═O)R₁; and R₁ can be a mono-substituted, poly-substituted or unsubstituted aryl (e.g., phenyl). In other embodiments, Y can be C—R₃, wherein R₃ can be selected from the group consisting of alkyl (e.g., methyl), alkoxy (e.g., methoxy), —C≡N, and halogen; B can be —C(═O)R₁; and R₁ can be a mono-substituted, poly-substituted or unsubstituted aralkyl such as a phenyl(C₁₋₄alkyl). In other embodiments, Y can be C—R₃, wherein R₃ can be selected from the group consisting of alkyl (e.g., methyl), alkoxy (e.g., methoxy), —C≡N, and halogen; B can be —C(═O)R₁; and R₁ can be a mono-substituted, poly-substituted or unsubstituted haloalkyl (e.g., CF₃). In some embodiments, Y can be C—R₃, wherein R₃ can be selected from the group consisting of alkyl, alkoxy, —C≡N, and halogen; B can be selected from the group consisting of —C(═O)R₁, —C(═O)OR₁—C(=Z)NR₁ₐR_{1b}, —C(=Z)N(OR₁ₐ)R_{1b}, and —C≡N; and R₂ and R₂ₐ can be taken together, along with the nitrogen atom to which they are attached, to form an unsubstituted or substituted heteroalicyclyl such as those described herein.

As to A, in some embodiments, A can be hydrogen. In other embodiments, A can be halogen. In still other embodiments, A can be cyano. In other embodiments, A can be a mono-substituted, poly-substituted or unsubstituted alkyl. In yet other embodiments, A can be a mono-substituted, poly-substituted or unsubstituted alkenyl. In some embodiments, A can be a mono-substituted, poly-substituted or unsubstituted alkynyl. In other embodiments, A can be a mono-substituted, poly-substituted or unsubstituted cycloalkyl. In yet other embodiments, A can be a mono-substituted, poly-substituted or unsubstituted cycloalkenyl. In still other embodiments, A can be a mono-substituted, poly-substituted or unsubstituted cycloalkynyl. In still other embodiments, A can be a mono-substituted, poly-substituted or unsubstituted aryl. In some embodiments, A can be a mono-substituted, poly-substituted or unsubstituted heteroaryl. In other embodiments, A can be a mono-substituted, poly-substituted or unsubstituted heteroalicyclyl. In yet other embodiments, A can be a mono-substituted, poly-substituted or unsubstituted aralkyl. In still other embodiments, A can be a mono-substituted, poly-substituted or unsubstituted heteroaralkyl. In yet other embodiments, A can be a mono-substituted, poly-substituted or unsubstituted (heteroalicyclyl)alkyl. In other embodiments, A can be a mono-substituted, poly-substituted or unsubstituted —C(=Z)R₁. In some embodiments, A can be a mono-substituted, poly-substituted or unsubstituted —C(=Z)OR₁. In other embodiments, A can be a mono-substituted, poly-substituted or unsubstituted —C(=Z)NR₁ₐR_{1b}. In yet other embodiments, A can be a mono-substituted, poly-substituted or unsubstituted —C(R₁)NR₁ₐ. In yet other embodiments, A can be a mono-substituted, poly-substituted or unsubstituted —NR₁ₐR_{1b}. In other embodiments, A can be a mono-substituted, poly-substituted or unsubstituted —N═CR₁ₐR_{1b}. In yet other embodiments, A can be a mono-substituted, poly-substituted or unsubstituted —N(R₁)—C(=Z)R₁. In some embodiments, A can be a mono-substituted, poly-substituted or unsubstituted —N(R₁)—C(=Z)NR₁ₐR_{1b}. In other embodiments, A can be a mono-substituted, poly-substituted or unsubstituted —S(O)NR₁ₐR_{1b}. In yet other embodiments, A can be a mono-substituted, poly-substituted or unsubstituted —S(O)₂NR₁ₐR_{1b}. In some embodiments, A can be a mono-substituted, poly-substituted or unsubstituted, —N(R₁)—S(═O)R₁. In other embodiments, A can be a mono-substituted, poly-substituted or unsubstituted —N(R₁)—S(═O)₂R₁. In yet other embodiments, A can be a mono-substituted, poly-substituted or unsubstituted —OR₁. In yet other embodiments, A can be a mono-substituted, poly-substituted or unsubstituted —SR₁. In some embodiments, A can be a mono-substituted, poly-substituted or unsubstituted, —OC(═O)R₁. In some embodiments, A can be a mono-substituted, poly-substituted or unsubstituted sulfinyl. In other embodiments, A can be a mono-substituted, poly-substituted or unsubstituted sulfonyl. In yet other embodiments, A can be a mono-substituted, poly-substituted or unsubstituted haloalkyl. In yet still other embodiments, A can be a mono-substituted, poly-substituted or unsubstituted haloalkoxy. In a preferred embodiment, A is hydrogen. In another preferred embodiment, A is an alkyl such as methyl.

In certain embodiments, A cannot be an aryl group. In some embodiments, A cannot be a heteroaryl group. In certain embodiments, A cannot be mono-substituted, poly-substituted or unsubstituted —C(═O)NR₁ₐR_{1b}, wherein R₁ₐ is hydrogen and R_{1b} is heteroaryl or heteroalicyclyl. In certain embodiments, A cannot be mono-substituted, poly-substituted or unsubstituted —C(═O)NR₁ₐR_{1b} wherein R₁ₐ is hydrogen and R_{1b} is heteroaryl or heteroaliyclyl such as thiazolyl, oxazolyl, isoxazolyl, 1,3,4-thiadiazolyl 1,2,4-thiadiazolyl, 1,3,4-oxadiazolyl, 1,2,4-oxadiazolyl, pyrazolyl, 1,2,4-triazolyl, tetrazolyl, 3-oxo-pyrazolyl, 3-oxo-imidazolyl, 3-oxo-thiazolyl, thiazolidinyl, pyridyl, pyrimidinyl, pyrazinyl, 1,3,5-triazinyl, 1,2,4-traizinyl, benyzlimidazolyl, 4-oxo-pyrimidyl, pyridazinyl and 2-oxo-pyrimidyl.

In certain embodiments when B is —C(═O)NR₁ₐR_{1b}, wherein R₁ₐ and/or R_{1b} are hydrogen, a mono-substituted, poly-substituted or unsubstituted variant selected from the group consisting of alkyl, cycloalkyl, and aryl, R₂ and/or R₂ₐ cannot be hydrogen, aminoalkyl, or alkylcarbonyl. In some embodiments when B is —C(═O)NR₁ₐR_{1b}, R₂ and R₂ₐ cannot be taken together, along with the nitrogen atom to which they are attached, to form a N-morpholinyl group. In some embodiments when B is —C(═O)NR₁ₐR_{1b}, R₂ and R₂ₐ cannot be taken together, along with the nitrogen atom to which they are attached, to form an unsubstituted heteroalicyclyl. In still other embodiments, B cannot be —C(═O)R₁, wherein R₁ is a cycloalkyl such as a C₃₋₈ cycloalkyl. In yet still other embodiments, B cannot be —C(═O)R₁, wherein R₁ is a cycloalkyl (e.g., a C₃₋₈ cycloalkyl) when R₂ and R₂ₐ are a mono- or di-substituted aminocarbonylalkyl, a mono- or di-substituted aminosulfonylalkyl, a mono- or di-substituted aminoalkyl. In certain embodiments, B cannot be mono-substituted, poly-substituted or unsubstituted —C(═O)N₁ₐR_{1b} wherein R₁ₐ is hydrogen and R_{1b} is heteroaryl or heteroalicyclyl. In certain embodiments, B cannot be mono-substituted, poly-substituted or unsubstituted —C(═O)NR₁ₐR_{1b} wherein R₁ₐ is hydrogen and R_{1b} is heteroaryl or heteroalicyclyl such as thiazolyl, oxazolyl, isoxazolyl, 1,3,4-thiadiazolyl, tetrazolyl, 3-oxo-pyrazolyl, 3-oxo-imidazolyl, 3-oxo-thiazolyl, thiazolidinyl, pyridyl, pyrimidinyl, pyrazinyl, 1,3,5-triazinyl, 1,2,4-triazinyl, benzylimidazolyl, 4-oxo-pyrimidyl, pyridazinyl and 2-oxo-pyrimidyl. In some embodiments, B cannot be —C(═O)R₁, wherein R₁ is an unsubstituted or substituted phenyl. In some embodiments, B cannot be —C(═O)R₁, wherein R₁ is an unsubstituted or substituted phenyl, when R₂ is hydrogen or C₁₋₄alkyl and R₂ₐ is —SO₂NR₁ₐR_{1b}, wherein R₁ₐ and R_{1b} are hydrogen or a C₁₋₄alkyl. In some embodiments, B cannot be —C(═O)NR₁ₐR_{1b} wherein R₁ₐ is hydrogen and R_{1b} is C₃₋₁₀alkyl, C₅₋₁₀carbocycle (e.g., unsubstituted C₅₋₁₀carbocycle or a C₅₋₁₀carbocycle substituted with methyl) when R₂ is hydrogen and R₂ₐ is C₁₋₄alkylsulfonyl or haloalkylsulfonyl such as CF₃SO₂—. In some embodiments, B cannot be a heteroalicyclyl. In certain embodiments, B cannot be piperidinyl or 1,2,3,6-tetrahydropyridinyl.

In some embodiment, A and B can be taken together to form an unsubstituted or substituted cycloalkyl. In other embodiments, A and B can be taken together to form an unsubstituted or substituted heteroalicyclyl.

In some of the embodiments, L can be an unsubstituted or substituted lower alkylene group. Preferably, L is ethylene, propylene, or butylene. More preferably, L is propylene. If L is substituted, suitable substituents without limitation are alkyl (e.g., methyl), alkenyl, halogen, haloalkyl (e.g., CF₃), alkoxy (e.g., methoxy), haloalkoxy, hydroxyl, and —CN. In some embodiments L cannot a monosubstituted lower alkylene group, wherein the lower alkylene is monosubstituted with a hydroxyl group, when R₂ and R₂ₐ are both hydrogen or methyl. In certain embodiments, L cannot a monosubstituted lower alkylene group, wherein the lower alkylene is monosubstituted with a branched alkyl group. In some embodiments, L can be taken together with R₃ to form a cycloalkyl, cycloalkenyl, cycloalkynyl, or heteroalicyclyl ring. In other embodiments, L cannot be taken together with R₃ to form a cycloalkyl, cycloalkenyl, cycloalkynyl, or heteroalicyclyl ring.

Some embodiments disclosed herein relate to a compound of Formula (I), in which any embodiment of A can be combined with any one or more embodiments of B, R₁, R₁ₐ, R_{1b}, R₂, R₂ₐ, R₃, R₃a, R_{3b}, R_{3c}, L, Y, Z, Q, n, m, R₄ₐ, R_{4b}, R_{4c}, R_{4d}, R₄ₑ, R_{4f}, and R_{4g}.

Other embodiments disclosed herein relate to a compound of Formula (I), in which any embodiment of B can be combined with any one or more embodiments of A, R₁, R₁ₐ, R_{1b}, R₂, R₂ₐ, R₃, R₃a, R_{3b}, R_{3c}, L, Y, Z, Q, n, m, R₄ₐ, R_{4b}, R_{4c}, R_{4d}, R₄ₑ, R_{4f}, and R_{4g}.

Still other embodiments disclosed herein relate to a compound of Formula (I), in which any embodiment of R₁ can be combined with any one or more embodiments of A, B, R₁ₐ, R_{1b}, R₂, R₂ₐ, R₃, R₃a, R_{3b}, R_{3c}, L, Y, Z, Q, n, m, R₄ₐR_{4b}, R_{4c}, R_{4d}, R₄ₑ, R_{4f}, and R_{4g}.

Yet still other embodiments disclosed herein relate to a compound of Formula (I), in which any embodiment of R₁ₐ can be combined with any one or more embodiments of A, B, R₁, R_{1b}, R₂, R₂ₐ, R₃, R₃a, R_{3b}, R_{3c}, L, Y, Z, Q, n, m, R₄ₐ, R_{4b}, R_{4c}, R_{4d}, R₄ₑ, R_{4f}, and R_{4g}.

Some embodiments disclosed herein relate to a compound of Formula (I), in which any embodiment of R_{1b} can be combined with any one or more embodiments of A, B, R₁, R₁ₐ, R₂, R₂ₐ, R₃, R₃a, R_{3b}, R_{3c}, L, Y, Z, Q, n, m, R₄ₐ, R_{4b}, R_{4c}, R_{4d}, R₄ₑ, R_{4f}, and R_{4g}.

Other embodiments disclosed herein relate to a compound of Formula (I), in which any embodiment of R₂ can be combined with any one or more embodiments of A, B, R₁, R₁ₐ, R_{1b}, R₂ₐ, R₃, R₃a, R_{3b}, R_{3c}, L, Y, Z, Q, n, m, R₄ₐ, R_{4b}, R_{4c}, R_{4d}, R₄ₑ, R_{4f}, and R_{4g}.

Still other embodiments disclosed herein relate to a compound of Formula (I), in which any embodiment of R₂ₐ can be combined with any one or more embodiments of A, B, R₁, R₁ₐ, R_{1b}, R₂, R₃, R₃a, R_{3b}, R_{3c}, L, Y, Z, Q, n, m, R₄ₐ, R_{4b}, R_{4c}, R_{4d}, R₄ₑ, R_{4f}, and R_{4g}.

Yet still other embodiments disclosed herein relate to a compound of Formula (I), in which any embodiment of R₃ can be combined with any one or more embodiments of A, B, R₁, R₁ₐ, R_{1b}, R₂, R₂ₐ, R₃ₐ, R_{3b}, R_{3c}, L, Y, Z, Q, n, m, R₄ₐ, R_{4b}, R_{4c}, R_{4d}, R₄ₑ, R_{4f}, and R_{4g}.

Other embodiments disclosed herein relate to a compound of Formula (I), in which any embodiment of R₃ₐ can be combined with any one or more embodiments of A, B, R₁, R₁ₐ, R_{1b}, R₂, R₂ₐ, R₃, R_{3b}, R_{3c}, L, Y, Z, Q, n, m, R₄ₐ, R_{4b}, R_{4c}, R_{4d}, R₄ₑ, R_{4f}, and R_{4g}.

Still other embodiments disclosed herein relate to a compound of Formula (I), in which any embodiment of R_{3b} can be combined with any one or more embodiments of A, B, R₁, R₁ₐ, R_{1b}, R₂, R₂ₐ, R₃, R₃ₐ, R_{3c}, L, Y, Z, Q, n m, R₄ₐ, R_{4b}, R_{4c}, R_{4d}, R₄ₑ, R_{4f}, and R_{4g}.

Yet still other embodiments disclosed herein relate to a compound of Formula (I), in which any embodiment of R_{3c} can be combined with any one or more embodiments of A, B, R₁, R₁ₐ, R_{1b}, R₂, R₂ₐ, R₃, R₃ₐ, R_{3b}, L, Y, Z, Q, n, m, R₄ₐ, R_{4b}, R_{4c}, R_{4d}, R₄ₑ, R_{4f}, and R_{4g}.

Some embodiments disclosed herein relate to a compound of Formula (I), in which any embodiment of L can be combined with any one or more embodiments of A, B, R₁, R₁ₐ, R_{1b}, R₂, R₂ₐ, R₃, R₃a, R_{3b}, R_{3c}, Y, Z, Q, n, m, R₄ₐ, R_{4b}, R_{4c}, R_{4d}, R₄ₑ, R_{4f}, and R_{4g}.

Other embodiments disclosed herein relate to a compound of Formula (I), in which any embodiment of Y can be combined with any one or more embodiments of A, B, R₁, R₁ₐ, R_{1b}, R₂, R₂ₐ, R₃, R₃a, R_{3b}, R_{3c}, L, Z, Q, n, m, R₄ₐ, R_{4b}, R_{4c}, R_{4d}, R₄ₑ, R_{4f}, and R_{4g}.

Still other embodiments disclosed herein relate to a compound of Formula (I), in which any embodiment of Z can be combined with any one or more embodiments of A, B, R₁, R₁ₐ, R_{1b}, R₂, R₂ₐ, R₃, R₃a, R_{3b}, R_{3c}, L, Y, Q, n, m, R₄ₐ, R_{4b}, R_{4c}, R_{4d}, R₄ₑ, R_{4f}, and R_{4g}.

Yet still other embodiments disclosed herein relate to a compound of Formula (I), in which any embodiment of Q can be combined with any one or more embodiments of A, B, R₁, R₁ₐ, R_{1b}, R₂, R₂ₐ, R₃, R₃a, R_{3b}, R_{3c}, L, Y, Z, n, m, R₄ₐ, R_{4b}, R_{4c}, R_{4d}, R₄ₑ, R_{4f}, and R_{4g}.

Some embodiments disclosed herein relate to a compound of Formula (I), in which any embodiment of n can be combined with any one or more embodiments of A, B, R₁, R₁ₐ, R_{1b}, R₂, R₂ₐ, R₃, R₃a, R_{3b}, R_{3c}, L, Y, Z, Q, m, R₄ₐ, R_{4b}, R_{4c}, R_{4d}, R₄ₑ, R_{4f}, and R_{4g}.

Other embodiments disclosed herein relate to a compound of Formula (I), in which any embodiment of m can be combined with any one or more embodiments of A, B, R₁, R₁ₐ, R_{1b}, R₂, R₂ₐ, R₃, R₃a, R_{3b}, R_{3c}, L, Y, Z, Q, n, R₄ₐ, R_{4b}, R_{4c}, R_{4d}, R₄ₑ, R_{4f}, and R_{4g}.

Still other embodiments disclosed herein relate to a compound of Formula (I), in which any embodiment of R₄ₐ can be combined with any one or more embodiments of A, B, R₁, R₁ₐ, R_{1b}, R₂, R₂ₐ, R₃, R₃a, R_{3b}, R_{3c}, L, Y, Z, Q, n, m, R_{4b}, R_{4c}, R_{4d}, R₄ₑ, R_{4f}, and R_{4g}.

Yet still other embodiments disclosed herein relate to a compound of Formula (I), in which any embodiment of R_{4b} can be combined with any one or more embodiments of A, B, R₁, R₁ₐ, R_{1b}, R₂, R₂ₐ, R₃, R₃a, R_{3b}, R_{3c}, L, Y, Z, Q, n, m, R₄ₐ, R_{4c}, R_{4d}, R₄ₑ, R_{4f}, and R_{4g}.

Some embodiments disclosed herein relate to a compound of Formula (I), in which any embodiment of R_{4c} can be combined with any one or more embodiments of A, B, R₁, R₁ₐ, R_{1b}, R₂, R₂ₐ, R₃, R₃a, R_{3b}, R_{3c}, L, Y, Z, Q, n, m, R₄ₐ, R_{4b}, R_{4d}, R₄ₑ, R_{4f}, and R_{4g}.

Other embodiments disclosed herein relate to a compound of Formula (I), in which any embodiment of R_{4d} can be combined with any one or more embodiments of A, B, R₁, R₁ₐ, R_{1b}, R₂, R₂ₐ, R₃, R₃a, R_{3b}, R_{3c}, L, Y, Z, Q, n, m, R₄ₐ, R_{4b}, R_{4c}, R₄ₑ, R_{4f}, and R_{4g}.

Still other embodiments disclosed herein relate to a compound of Formula (I), in which any embodiment of R₄₁ can be combined with any one or more embodiments of A, B, R₁, R₁ₐ, R_{1b}, R₂, R₂ₐ, R₃, R₃a, R_{3b}, R_{3c}, L, Y, Z, Q, n, m, R₄ₐ, R_{4b}, R_{4c}, R_{4d}, R_{4f}, and R_{4g}.

Yet still other embodiments disclosed herein relate to a compound of Formula (I), in which any embodiment of R_{4f} can be combined with any one or more embodiments of A, B, R₁, R₁ₐ, R_{1b}, R₂, R₂ₐ, R₃, R₃a, R_{3b}, R_{3c}, L, Y, Z, Q, n, m, R₄ₐ, R_{4b}, R_{4c}, R_{4d}, R₄ₑ, and R_{4g}.

Some embodiments disclosed herein relate to a compound of Formula (I), in which any embodiment of R_{4g} can be combined with any one or more embodiments of A, B, R₁, R₁ₐ, R_{1b}, R₂, R₂ₐ, R₃, R₃a, R_{3b}, R_{3c}, L, Y, Z, Q, n, m, R₄ₐ, R_{4b}, R_{4c}, R_{4d}, R₄ₑ, and R_{4f}.

Definitions

Unless defined otherwise, all technical and scientific terms used herein have the same meaning as is commonly understood by one of ordinary skill in the art to which this invention belongs. All patents, applications, published applications and other publications referenced herein are incorporated by reference in their entirety. In the event that there are plurality of definitions for a term herein, those in this section prevail unless stated otherwise

As used herein, any “K” group(s) such as, without limitation, R₁, R₁ₐ and R_{1b}, represent substituents that can be attached to the indicated atom. A non-limiting list of R groups include hydrogen, alkyl, alkenyl, alkynyl, cycloalkyl, cycloalkenyl, cycloalkynyl, aryl, heteroaryl, and heteroalicyclyl. An R group may be substituted or unsubstituted. If two “R” groups are covalently bonded to the same atom or to adjacent atoms, then they may be “taken together” as defined herein to form a cycloalkyl, aryl, heteroaryl or heteroalicyclyl group. For example, without limitation, if Rₐ and R_{b} of an NRₐR_{b} group are indicated to be “taken together”, it means that they are covalently bonded to one another at their terminal atoms to form a ring that includes the nitrogen:

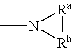

Whenever a group of this invention is described as being “optionally substituted” that group may be unsubstituted or substituted with one or more of the indicated substituents. Likewise, when a group is described as being “unsubstituted or substituted” if substituted, the substituent may be selected from one or more of the indicated substituents.

Unless otherwise indicated, when a substituent is deemed to be “optionally substituted,” or “substituted” it is meant that the substitutent is a group that may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. The protecting groups that may form the protective derivatives of the above substituents are known to those of skill in the art and may be found in references Greene and Wuts, Protective Groups in Organic Synthesis, 3^{rd} Ed., John Wiley & Sons, New York, N.Y., 1999, which is hereby incorporated by reference in its entirety.

As used herein, “Cₘ to Cₙ” in which “m” and “n” are integers refers to the number of carbon atoms in an alkyl, alkenyl or alkynyl group or the number of carbon atoms in the ring of a cycloalkyl or cycloalkenyl group. That is, the alkyl, alkenyl, alkynyl, ring of the cycloalkyl or ring of the cycloalkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl, alkenyl, alkynyl, cycloalkyl or cycloalkenyl group, the broadest range described in these definitions is to be assumed.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substitutent may be substituted with one of the above substituents.

As used herein, “alkenyl” refers to an alkyl group that contains in the straight or branched hydrocarbon chain one or more double bonds. An alkenyl group of this invention may be unsubstituted or substituted. When substituted, the substituent(s) may be selected from the same groups disclosed above with regard to alkyl group substitution.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. Examples of heteroaryl rings include, but are not limited to, furan, thiophene, phthalazine, pyrrole, oxazole, thiazole, imidazole, pyrazole, isoxazole, isothiazole, triazole, thiadiazole, pyridine, pyridazine, pyrimidine, pyrazine and triazine. A heteroaryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylethyl, 3-phenyl)propyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylmethyl, 3-thienylmethyl, furylmethyl, thienylethyl, pyrrolylalkyl, pyridylalkyl, isoxazollylalkyl, and imidazolylalkyl, and their substituted as well as benzo-fused analogs.

“Lower alkylene groups” are straight-chained tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—(CH₂)₄—) groups. A lower alkylene group may be substituted or unsubstituted.

As used herein, “alkylidene” refers to a divalent group, such as —CR′R″, which is attached to one carbon of another group, forming a double bond, Alkylidene groups include, but are not limited to, methylidene (═CH₂) and ethylidene (═CHCH₃). As used herein, “arylalkylidene” refers to an alkylidene group in which either R′ and R″ is an aryl group. An alkylidene group may be substituted or unsubstituted.

As used herein, “alkoxy” refers to the formula —OR wherein R is an alkyl is defined as above, e.g. methoxy, ethoxy, n-propoxy, T-methylethoxy (isopropoxy), n-butoxy, iso-butoxy, sec-butoxy, tert-butoxy, amoxy, tert-amoxy and the like. An alkoxy may be substituted or unsubstituted.

As used herein, “alkylthio” refers to the formula —SR wherein R is an alkyl is defined as above, e.g. methylmercapto, ethylmercapto, n-propylmercapto, I methylethylmercapto (isopropylmercapto), n-butylmercapto, iso-butylmereapto, sec-butylmercapto, tert-butylmereapto, and the like. An alkylthio may be substituted or unsubstituted.

As used herein, “aryloxy” and “arylthio” refers to RO— and RS—, in which R is an aryl, such as but not limited to phenyl. Both an aryloxyl and arylthio may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted. An acyl may be substituted or unsubstituted.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups of this invention may range from C₃ to C₁₀, in other embodiments it may range from C₃ to C₆. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). When composed of two or more rings, the rings may be connected together in a fused, bridged or spiro-connected fashion. A cycloalkenyl group of this invention may be unsubstituted or substituted. When substituted, the substituent(s) may be an alkyl or selected from the groups disclosed above with regard to alkyl group substitution unless otherwise indicated.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group of this invention may be unsubstituted or substituted. When substituted, the substituent(s) may be an alkyl or selected from the groups disclosed above with regard to alkyl group substitution unless otherwise indicated.

A “(cycloalkyl)alkyl” is a cycloalkyl group connected, as a substituent, via a lower alkylene group. The lower alkylene and cycloalkyl of a (cycloalkyl)alkyl may be substituted or unsubstituted. Examples include but are not limited cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like.

A “(cycloalkenyl)alkyl” is a cycloalkenyl group connected, as a substituent, via a lower alkylene group. The lower alkylene and cycloalkenyl of a (cycloalkenyl)alkyl may be substituted or unsubstituted.

A “(cycloalkynyl)alkyl” is a cycloalkynyl group connected, as a substituent, via a lower alkylene group. The lower alkylene and cycloalkynyl of a (cycloalkynyl)alkyl may be substituted or unsubstituted.

As used herein, “heteroalicyclic” or “heteroalicyclyl” refers to a stable 3- to 18 membered ring which consists of carbon atoms and from one to five heteroatoms selected from the group consisting of nitrogen, oxygen and sulfur. For the purpose of this invention, the “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups of this invention may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl N-Oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone.

An “(heteroalicyclyl)alkyl” is a heterocyclic or a heterocyclyl group connected, as a substituent, via a lower alkylene group. The lower alkylene and heterocyclic or a heterocyclyl of a (heteroalicyclyl)alkyl may be substituted or unsubstituted. Examples include but are not limited 4-methyltetrahydro-2H-pyran, substituted 4-methyltetrahydro-2H-pyran, 4-ethylpiperidine, 4-propylpiperidine, 4-methyltetrahydro-2H-thiopyran, and 4-methyl-1,3-thiazinane.

As used herein, “halo” or “halogen” refers to F (fluoro), Cl (chloro), Br (bromo) or I (iodo).

As used herein, “haloalkyl” refers to an alkyl group in which one or more of the hydrogen atoms are replaced by halogen. Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an “RO—” group in which R is a haloalkyl group. Such groups include but are not limited to, chloromethoxy, fluoromethoxy, difluoromethoxy, trifluoromethoxy and 1-chloro-2-fluoromethoxy, 2-fluoroisobutyoxy. A haloalkoxy may be substituted or unsubstituted.

An “O-carboxy” group refers to an “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

A “C-carboxy” group refers to a “—C(—O)R” group in which R can be the same as defined with respect to O-carboxy. A C-carboxy may be substituted or unsubstituted.

A “trihalomethanesulfonyl” group refers to an “X₃CSO₂—” group wherein X is a halogen.

A “cyano” group refers to a “—CN” group.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group in which R can be the same as defined with respect to O-carboxy, A sulfinyl may be substituted or unsubstituted.

A “sulfonyl” group refers to an “SO₂R” group in which R can be the same as defined with respect to O-carboxy. A sulfonyl may be substituted or unsubstituted.

An “S-sulfonamido” group refers to an “—SO₂NR_{A}R_{B}” group in which R_{A} and R_{B} can be the same as defined with respect to O-carboxy. An S-sulfonamido may be substituted or unsubstituted.

An “N-sulfonamido” group refers to an “RSO₂N(R_{A})—” group in which R and R_{A} can be the same as defined with respect to O-carboxy. A sulfonyl may be substituted or unsubstituted.

A “trihalomethanesulfonamido” group refers to an “X₃CSO₂N(R)—” group with X as halogen and R can be the same as defined with respect to O-carboxy. A trihalomethanesulfonamido may be substituted or unsubstituted.

An “O-carbamyl” group refers to an “—OC(═O)NR_{A}R_{B}” group in which R_{A} and R_{B} can be the same as defined with respect to O-carboxy. An O-carbamyl may be substituted or unsubstituted.

An “N-carbamyl” group refers to an “ROC(═O)NR_{A}—” group in which R and R_{A} can be the same as defined with respect to O-carboxy. An N-carbamyl may be substituted or unsubstituted.

An “O-thiocarbamyl” group refers to an “—OC(═S)—NR_{A}R_{B}” group in which R_{A} and R_{B} can be the same as defined with respect to O-carboxy. An O-thiocarbamyl may be substituted or unsubstituted.

An “N-thiocarbamyl” group refers to an “ROC(═S)NR_{A}—” group in which R and R_{A} can be the same as defined with respect to O-carboxy. An N-thiocarbamyl may be substituted or unsubstituted.

A “C-amido” group refers to a “—C(═O)NR_{A}R_{B}” group in which R_{A} and R_{B} can be the same as defined with respect to O-carboxy. A C-amido may be substituted or unsubstituted.

An “N-amido” group refers to an “RC(═O)NR_{A}—” group in which R and R_{A} can be the same as defined with respect to O-carboxy. An N-amido may be substituted or unsubstituted.

An “ester” refers to a “—C(═O)OR” group in which R can be the same as defined with respect to O-carboxy. An ester may be substituted or unsubstituted.

As used herein, an “amide” refers to a “—C(═O)NR_{A}R_{B}” group in which R_{A} and R_{B} can be the same as R defined with respect to O-carboxy.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999).

Where the numbers of substituents is not specified (e.g. haloalkyl), there may be one or more substituents present. For example “haloalkyl” may include one or more of the same or different halogens. As another example, “C1-C3 alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As employed herein, the following terms have their accepted meaning in the chemical literature.

AcOH Acetic acid

anhyd anhydrous

CDI 1,1′-carbonyldiimidazole

DCM Dichloromethane

DMF N,N-Dimethylformamide

DMAP 4-Dimethylaminopyridine

DMSODimethyl sulfoxide

EDCl 1-(3-Dimethylaminopropyl)-3-ethylcarbodiimide hydrochloride

Et₂O Diethyl ether

EtOAc Ethyl acetate

EtOH Ethanol

McOH Methanol

MeCN Acetonitrile

NH₄OAc Ammonium acetate

NMM N-Methylmorpholine

HOBt 1-Hydroxybenztriazole

Pd/C Palladium on activated carbon

TEA Triethylamine

THF Tetrahydrofuran

uW, MW Microwave reactor chemistry

It is understood that, in any compound of this invention having one or more chiral centers, if an absolute stereochemistry is not expressly indicated, then each center may independently be of R-configuration or S-configuration or a mixture thereof. Thus, the compounds provided herein may be enantiomerically pure or be stereoisomeric or diastereomeric mixtures. In addition it is understood that, in any compound of this invention having one or more double bond(s) generating geometrical isomers that can be defined as E or Z each double bond may independently be E or Z a mixture thereof. Likewise, all tautomeric forms are also intended to be included.

As used herein, “pharmaceutically acceptable salt” refers to a salt of a compound that does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methanesulfonates, ethanesulfonates, p-toluenesulfonates and salicylates.

Pharmaceutically acceptable solvates and hydrates are complexes of a compound with one or more solvent of water molecules, or 1 to about 100, or 1 to about 10, or one to about 2, 3 or 4, solvent or water molecules.

As used herein, a “prodrug” refers to a compound that may not be pharmaceutically active but that is converted into an active drug upon in vivo administration. The prodrug may be designed to alter the metabolic stability or the transport characteristics of a drug, to mask side effects or toxicity, to improve the flavor of a drug or to alter other characteristics or properties of a drug. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to release the active parent compound. By virtue of knowledge of pharmacodynamic processes and drug metabolism in vivo, those skilled in the art, once a pharmaceutically active compound is known, can design prodrugs of the compound (see, e.g. Nogrady (1985) Medicinal Chemistry A Biochemical Approach, Oxford University Press, New York, pages 388-392)

As used herein, the term “complement” refers to a oligonucleotide or polynucleotide that hybridizes by base-pairing, adenine to tyrosine and guanine to cytosine, to another oligonucleotide.

As used herein, to “modulate” the activity of a Ghrelin receptor means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all, even in the presence of a natural binding partner. A natural binding partner is an endogenous molecule that is an agonist for the receptor.

As used herein, to “detect” changes in the activity of a Ghrelin receptor or of a Ghrelin receptor sub-type refers to the process of analyzing the result of an experiment using whatever analytical techniques are best suited to the particular situation. In some cases simple visual observation may suffice, in other cases the use of a microscope, visual or UV light analyzer or specific protein assays may be required. The proper selection of analytical tools and techniques to detect changes in the activity of a Ghrelin receptor or a Ghrelin receptor sub-type are well-known to those skilled in the art.

An “agonist” is defined as a compound that increases the basal activity of a receptor (i.e. signal transduction mediated by the receptor).

As used herein, “partial agonist” refers to a compound that has an affinity for a receptor but, unlike an agonist, when bound to the receptor it elicits only a fractional degree of the pharmacological response normally associated with the receptor even if a large number of receptors are occupied by the compound.

An “inverse agonist” is defined as a compound, which reduces, or suppresses the basal activity of a receptor, such that the compound is not technically an antagonist but, rather, is an agonist with negative intrinsic activity.

As used herein, “antagonist” refers to a compound that binds to a receptor to form a complex that does not give rise to any response, as if the receptor was unoccupied. An antagonist attenuates the action of an agonist on a receptor. An antagonist may bind reversibly or irreversibly, effectively eliminating the activity of the receptor permanently or at least until the antagonist is metabolized or dissociates or is otherwise removed by a physical or biological process.

As used herein, “IC₅₀” refers to an amount, concentration of dosage of a particular test compound that achieves a 50% inhibition of a maximal response, such as modulation of GPCR, including Ghrelin receptor, activity, in an assay that measures such response in an assay that measures such response for example but not limited to R-SAT® described herein.

As used herein, “EC₅₀” refers to an dosage, concentration or amount of a particular test compound that elicits a dose-dependent respons at 50% of maximal expression of a particular response that is induced, provoked or potentiated by the particular test compound, in an assay that measures such response for example but not limited to R-SAT® described herein.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated in order to attempt to cure, or at least ameliorate the effects of, a particular disease or disorder or to prevent the disease or disorder from occurring in the first place.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment or therapy. Furthermore, treatment may include acts that may worsen the patient's overall feeling of well-being or appearance.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Synthesis

General synthetic routes to the compounds of this invention are shown in Scheme 1-11. The routes shown are illustrative only and are not intended, nor are they to be construed, to limit the scope of this invention in any manner whatsoever. Those skilled in the art will be able to recognize modifications of the disclosed synthesis and to devise alternate routes based on the disclosures herein; all such modifications and alternate routes are within the scope of this invention. 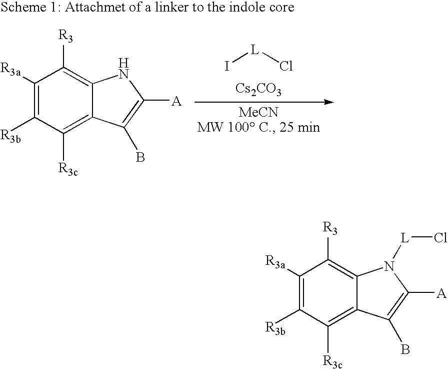 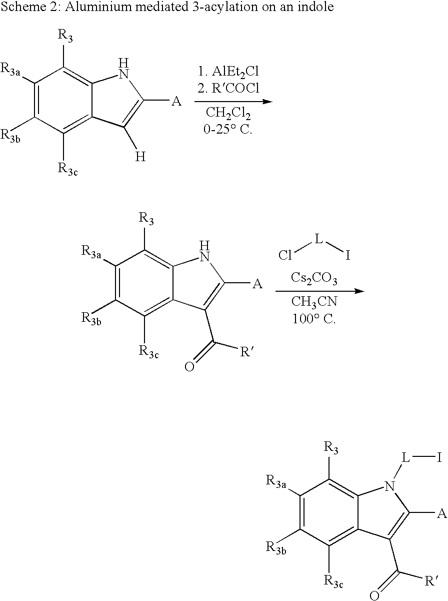 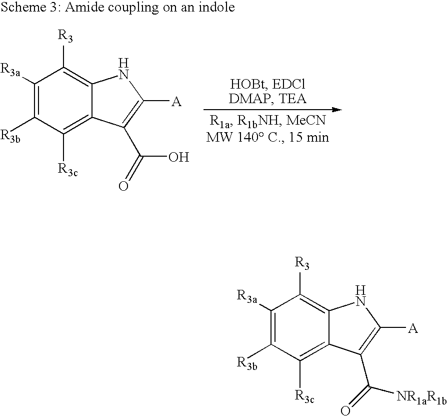 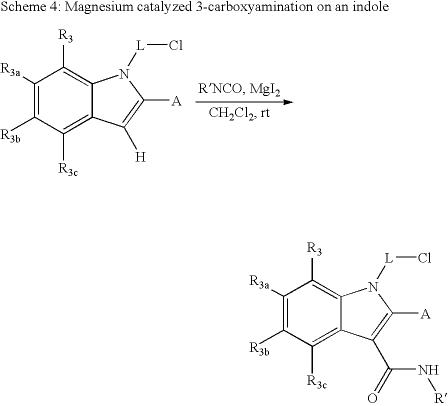 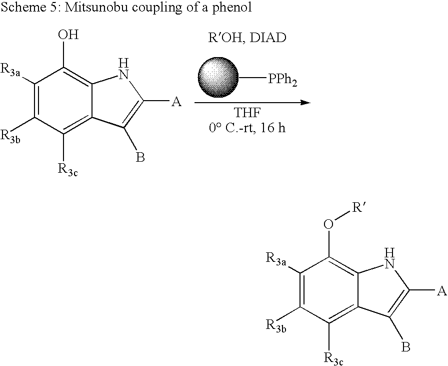 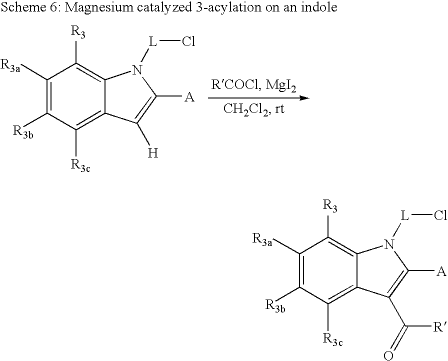 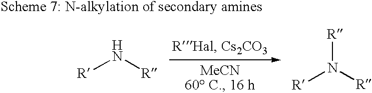 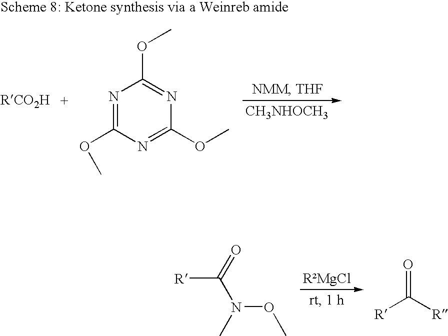 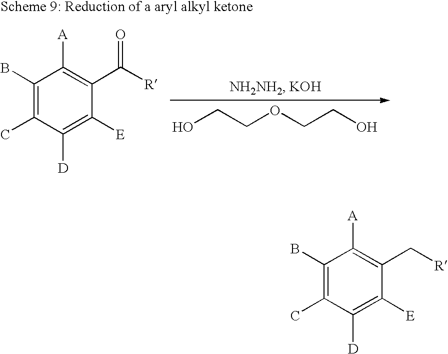 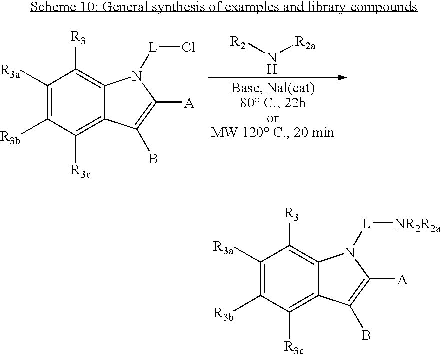 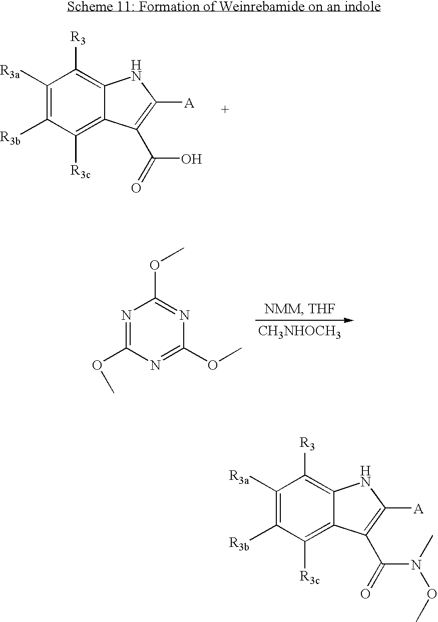
Methods of Use

Some embodiments disclosed herein relate to methods for treating or preventing diseases or conditions by administering one or more compounds of Formula I and/or a compound described herein. A non-limiting list of diseases or conditions include but are not limited to obesity, an obesity-associated disorder, a metabolic disorder, metabolic syndrome, an endocrine disorder, an appetite disorder, an eating disorder, an eating disorder requiring appetite control, atherosclerosis, diabetes, diabetes mellitus, high cholesterol, hyperlipidemia, cachexia, anorexia, bulimia, inflammation, a chronic inflammatory disorder, rheumatoid arthritis, asthma, psoriasis, a cardiovascular disorder, angina, cardiac ischemia, cardiac failure, heart disease, congestive heart failure, ischemic heart disease, chronic heart disease, hemorrhagic shock, septic shock, cirrhosis, a neurological disorder, anxiety, depression, an attention deficit disorder, a memory disorder, a cognitive disorder, a gastrointestinal disorder, reduced gastric motility, reduced gastric and intestinal motility, excessive gastric motility, post-operative gastric ileus, delayed gastric emptying, delayed gastric emptying due to diabetes, delayed gastric emptying post-operatively, short bowel syndrome, a gastric ulcer, nausea, emesis, diarrhea, gastroparesis, diabetic gastroparesis, opioid-induced bowel dysfunction, chronic intestinal pseudoobstruction, a sleep disorder, insomnia, a hyperproliferative disorder, cancer, cancer cachexia, dwarfism, osteoporosis, a catabolic state, somatopause, osteopenia, a disorder of the pancreas, a hormone deficiency, gastrointestinal dumping syndrome, postgastroenterectomy syndrome, celiac disease, AIDS, wasting, age-related decline in body composition, hypertension, retinopathy, dyslipidemia, a gall stone, osteoarthritis, congestive heart failure, insulin resistance, burn, wound, protein loss, sexual dysfunction, a central nervous system disorder, a genetic disorder, irritable bowel syndrome (IBS), non-ulcer dyspepsia, Crohn's disease, a gastroesophogeal reflux disorder, constipation, ulcerative colitis, pancreatitis, infantile hypertrophic pyloric stenosis, carcinoid syndrome, malabsorption syndrome, atrophic colitis, gastritis, gastric stasis, frailty, acromegaly, and protein loss.

Other embodiments disclosed herein relate to methods for treating impaired or risk of impaired wound healing, impaired or risk of impaired recovery from burns, impaired or risk of impaired recovery from surgery, impaired or risk of impaired muscle strength, impaired or risk of impaired mobility, altered or risk of altered skin thickness, impaired or risk of impaired metabolic homeostasis, or impaired or risk of impaired renal homeostasis.

Still other embodiments disclosed herein relate to methods for facilitating neonatal development, stimulating growth hormone release in humans, maintaining muscle strength and function in humans, reversing or preventing of frailty in humans, preventing of catabolic side effects of glucocorticoids, treating osteoporosis, stimulating and increasing muscle mass and/or muscle strength, stimulating the immune system, attenuating protein catabolic response, accelerating wound healing, accelerating bone fracture repair, treating renal failure or insufficiencies resulting in growth retardation, treating short stature, treating obesity and growth retardation, accelerating the recovery and reducing hospitalization of burn patients, treating intrauterine growth retardation, treating skeletal dysphasia, treating hypercortisolism, treating Cushing's syndrome, inducing pulsatile growth hormone release, replacing growth hormone in stressed patients, treating osteochondrodysplasias, treating Noonans syndrome, treating schizophrenia, treating depression, treating Alzheimer's disease, treating emesis, treating memory loss, treating reproduction disorders, treating delayed wound healing, treating psychosocial deprivation, treating pulmonary dysfunction, treating ventilator dependency; attenuating protein catabolic response, reducing cachexia and protein loss, treating hyperinsulinemia, improving ovulation induction, stimulating thymic development, preventing thymic function decline, treating immunosuppressed patients, improving muscle mobility, maintaining skin thickness, promoting metabolic homeostasis, promoting renal homeostasis, stimulating osteoblasts, stimulating bone remodeling, stimulating cartilage growth, stimulating the immune system in companion animals, treating disorders of aging in companion animals, promoting growth in livestock, and/or stimulating wool growth in sheep.

In one embodiment, a method of treating or preventing a disorder or condition comprises administering to a subject a therapeutically effective amount of a compound of Formula I and/or a compound described herein, for the purpose of alleviating and/or controlling the symptoms associated with these disorders or conditions.

In still another embodiment, the compound of Formula I and/or a compound described herein can modulate, agonize, inverse agonize, and/or antagonize a ghrelin receptor. In some embodiments, the compound of Formula I and/or a compound described herein can inverse agonize, and/or antagonize a ghrelin receptor.

Some embodiments described herein relate to the treatment of an eating disorder or condition related to an eating disorder. Treatment of eating disorders can include controlling the symptoms observed during these disorder or conditions, such as, for example, increased appetite and binge eating. In one embodiment, a method of treating an eating disorder or condition comprises administering a therapeutically effective amount of a compound of Formula I and/or a compound described herein, to a subject for the purpose of treating eating disorders requiring appetite control. In another embodiment, a method of treating an eating disorder or condition comprises administering a therapeutically effective amount of a compound of Formula I and/or a compound described herein to a subject for the purpose of treating a subject suffering from a symptom of an eating disorder requiring appetite control. In still another embodiment, a method of treating an eating disorder or condition comprises administering a therapeutically effective amount of a compound of Formula I and/or a compound described herein to a subject for the purpose of treating obesity and disorders associated with obesity. A non-limiting list of eating disorders and conditions associated with eating disorders includes obesity, metabolic syndrome, appetite disorders, cachexia, anorexia, bulemia, high cholesterol, hyperlipidemia, heart disease, atherosclerosis, and diabetes.

Other embodiments described herein relate to methods comprising administering a therapeutically effective amount of a compound of Formula I and/or a compound described herein to a subject for the purpose of promoting weight loss in a subject in need thereof. Promotion of weight loss can include reversing anabolic states.

Still other embodiments described herein relate to a method of preventing weight gain or weight loss in a subject comprising administering to a subject a therapeutically effective amount of a compound of Formula I and/or a compound described herein. Weight gain can be caused from a medication the subject is taking such as insulin, thiazolidinedione, sulfonylurea, corticosteroid, progestational steroid, antihistamine, alpha-adrenergic blocker, beta-adrenergic blocker, an antidepressant (e.g., a tricyclic antidepressant, selective serotonin reuptake inhibitor, a monoamine inhibitor, lithium), antipsychotic, and anticonvulsant. Weight loss can be caused by chemotherapy, radiation therapy, temporary immobilization, permanent immobilization or dialysis. In one embodiment, the compound of Formula I and/or a compound described herein can be used to prevent weight gain following weight loss by a subject.

Yet still other embodiments described herein relate to methods comprising administering a therapeutically effective amount of a compound of Formula I and/or a compound described herein to a subject for the purpose of maintaining the weight of a subject in need thereof.

Some embodiments disclosed herein relate to a method for the treatment of post-operative ileus and/or cachexia comprising comprise administering to the subject a pharmaceutically effective amount of a compound of Formula I and/or a compound described herein. Causes of post-operative ileus and/or cachexia include but are not limited to cancer, AIDS, cardiac disease and renal disease, gastroparesis, such as that resulting from type I or type II diabetes, other gastrointestinal disorders, growth hormone deficiency, bone loss, and other age-related disorders.

Other embodiments described herein relate to methods comprising administering a therapeutically effective amount of a compound of Formula I and/or a compound described herein to a subject for the purpose of treating a gastrointestinal disorder. Treatment of gastrointestinal disorders can include reversing the symptoms observed with these syndromes. Such symptoms can include loss of gastric motility or excessive gastric motility. Gastrointestinal disorders treatable by the methods of the present invention include, but are not limited to, reduced gastric and intestinal motility, post-operative gastric ileus, delayed gastric emptying, delayed gastric emptying due to diabetes, delayed gastric emptying post-operatively, short bowel syndrome, a gastric ulcer, nausea, emesis, and/or diarrhea.

Yet other embodiments described herein relate to methods comprising administering a therapeutically effective amount of a compound of Formula I and/or a compound described herein to a subject for the purpose of treating a cardiovascular disorder. Cardiovascular disorders treatable by the methods of the present invention include, but are not limited to, angina, cardiac ischemia, cardiac failure, heart disease, and related vascular disorders like atherosclerosis. In another embodiment, the methods of the present invention can also be effective in reducing cardiac afterload and/or increasing cardiac output.

Yet still other embodiments described herein relate to methods comprising administering a therapeutically effective amount of a compound of Formula I and/or a compound described herein to a subject for the purpose of treating a sleep disorder such as insomnia or narcolepsy. In one embodiment, the methods of improving sleep architecture, facilitating induction of sleep, and/or improving the quality of sleep comprises administering a therapeutically effective amount of a compound of Formula I and/or a compound described herein to a subject. In another embodiment, the methods of improving sleep architecture, facilitating induction of sleep, and/or improving the quality of sleep comprises administering a therapeutically effective amount of a compound of Formula I and/or a compound described herein, and a sleep agent such as ambien®, lunesta®, doxepin, indiplon, gaboxadol, and N-(4-fluorobenzyl)-N-(1-methylpiperidin-4-yl)-N′-(4-(2-methylproploxy)phenylmethyl)carbamide. In still another embodiment, the method for maintaining the sleep of a subject comprises administering a therapeutically effective amount of a compound of Formula I and/or a compound described herein. In yet still another embodiment, the method for maintaining the sleep of a subject comprises administering a therapeutically effective amount of a compound of Formula I and/or a compound described herein in combination with a sleep agent (e.g., ambien®, lunesta®t, doxepin, indiplon, gaboxadol, and N-(4-fluorobenzyl)-N-(1-methylpiperidin-4-yl)-N′-(4-(2-methylproploxy)phenylmethyl)carbamide. In one embodiment, the method for facilitating alertness or awakefulness comprises administering a therapeutically effective amount of a compound of Formula I and/or a compound described herein. In another embodiment, the method for facilitating alertness or wakefulness comprises administering a therapeutically effective amount of a compound of Formula I and/or a compound described herein to a subject, wherein the subject can be taking an agent that causes drowsiness or induces sleep (e.g., a sedative, ambien®, lunesta®, doxepin, or gaboxadol).

Some embodiments described herein relate to methods comprising administering a therapeutically effective amount of a compound of Formula I and/or a compound described herein to a subject for the purpose of treating a hyperproliferative disorder such as a tumor, cancer, and a neoplastic disorder, as well as a premalignant and non-neoplastic or non-malignant hyperproliferative disorder. In another embodiment, the methods of the present invention can also be effective in controlling unwanted cellular proliferation associated with a cancer. A non-limiting list of hyperproliferative disorders include but are not limited to malignant disorders such as breast cancers, osteosarcomas, angiosarcomas, fibrosarcomas and other sarcomas, leukemias, lymphomas, sinus tumors, ovarian cancers, uretal cancers, bladder cancers, prostate cancers, other genitourinary cancers, colon cancers, esophageal cancers, stomach cancers, other gastrointestinal cancers, lung cancers, myelomas, pancreatic cancers, liver cancers, kidney cancers, endocrine cancers, gliomas, neuroblastomas, skin cancers, brain cancers, and central and peripheral nervous (CNS) system tumors.

Other embodiments described herein relate to methods comprising administering a therapeutically effective amount of a compound of Formula I and/or a compound described herein to a subject for the purpose of diagnosing a hormone deficiency (e.g., production of a growth hormone, ACTH, cortisol, insulin-like growth factor 1 (IGF-1), and/or prolactin) In another embodiment, the methods of the present invention can also be effective in modulating hormone production.

Yet other embodiments described herein relate to methods comprising administering a therapeutically effective amount of a compound of Formula I and/or a compound described herein to a subject for the purpose of treating a hormone deficiency. Hormone deficiency disorders treatable by the methods of the present invention include, but are not limited to, deficiencies in producing growth hormone, ACTH, cortisol, insulin-like growth factor 1 (IGF-1), and/or prolactin.

Yet still other embodiments described herein relate to methods comprising administering a therapeutically effective amount of a compound of Formula I and/or a compound described herein to a subject for the purpose of treating dwarfism, osteoporosis, a catabolic state, somatopause, and/or osteopenia.

Some embodiments described herein relate to methods comprising administering a therapeutically effective amount of a compound of Formula I and/or a compound described herein to a subject for the purpose of treating a disorder of the pancreas.

Other embodiments described herein relates to a method of controlling the level of glucose in a subject comprising administering a therapeutically effective amount of a compound of Formula I and/or a compound described herein to a subject.

Yet other embodiments described herein relates to a method of treating diabetes in a subject comprising administering a therapeutically effective amount of a compound of Formula I and/or a compound described herein to a subject.

Yet still other embodiments described herein relate to methods comprising administering a therapeutically effective amount of a compound of Formula I and/or a compound described herein to a subject for the purpose of treating a neurological disorder, anxiety, depression, an attention deficit disorder, a memory disorder, and/or a cognitive disorder. In another embodiment, the methods of the present invention can also be effective in relieving symptoms of anxiety and/or improving memory. In one embodiment, a compound of Formula I and/or a compound described herein can be used to alleviate or treat a symptom associated with a neurological disorder comprising administering to a subject with altered cognition a compound of Formula I and/or a compound described herein.

Some embodiments described herein relate to methods comprising administering a therapeutically effective amount of a compound of Formula I and/or a compound described herein to a subject for the purpose of treating inflammation. The causes of the inflammation include but are not limited to a chronic inflammatory disorder, rheumatoid arthritis, asthma, an allergy, and/or psoriasis.

Other embodiments disclosed herein relate to methods for treating diseases or conditions by administering one or more compounds of Formula I and/or a compound described herein comprising identifying a subject in need of treatment or prevention and administering to the subject a therapeutically effective amount of a compound of Formula I and/or a compound described herein.

One embodiment described herein relates a method of identifying a compound which regulates activity of the Ghrelin receptor by culturing cells that express the Ghrelin receptor; incubating the cells with at least one compound of Formula I and/or a compound described herein as defined herein; and determining any change in activity of the Ghrelin receptor so as to identify a compound of Formula I and/or a compound described herein which regulates activity of the Ghrelin receptor.

In any of the methods described herein, in some embodiments, the compound of Formula (I) or a solvate, a polymorph, a metabolite, or a pharmaceutically acceptable salt or prodrug thereof, has the structure described herein provided that when R₂ and R₂ₐ are taken together, along with the nitrogen atom to which they are attached, form a substituted heteroalicyclyl, wherein the substituted heteroalicyclyl is 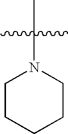
substituted with n-butyl at the para-position, then B cannot be selected from the group consisting of methyl, —C(═O)R₁, and —CH₂OH, wherein R₁ is hydrogen or methyl; or A cannot be methyl. In any of the methods described herein, in other embodiments, the compound of Formula (I) or a solvate, a polymorph, a metabolite, or a pharmaceutically acceptable salt or prodrug thereof, has the structure described herein provided that when R₂ and R₂ₐ are taken together, along with the nitrogen atom to which they are attached, form a substituted heteroalicyclyl, wherein the substituted heteroalicyclyl is 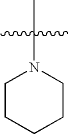
substituted with an alkyl, such as n-butyl, then A, R₃, R₃ₐ, R_{3b}, and R_{3c} cannot all be hydrogen. In any of the methods described herein, in some embodiments, the compound of formula I can be selected from the group consisting of: 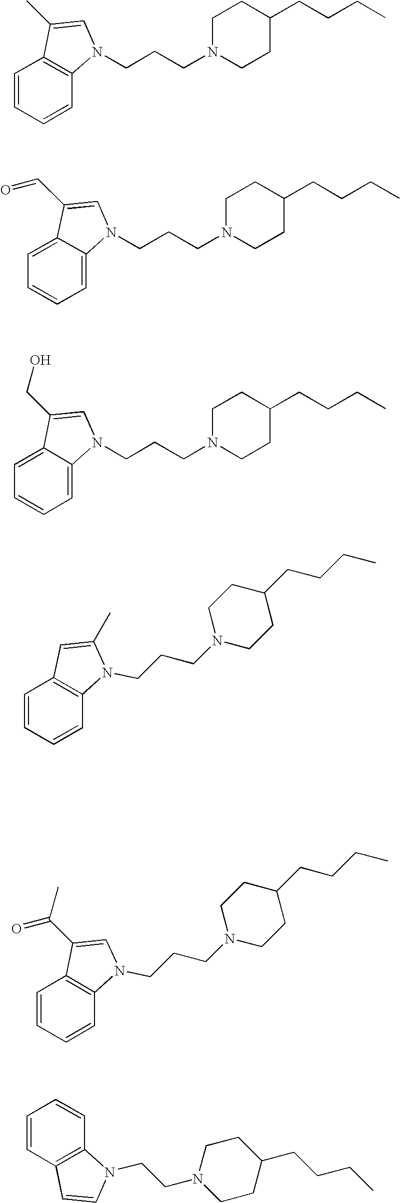
Pharmaceutical Compositions

Another embodiment described herein relates to a pharmaceutical composition comprising a compound of Formula I and/or a compound described herein, and a physiologically acceptable carrier, diluent, or excipient, or a combination thereof. In some embodiments, a pharmaceutical composition comprises a compound of Formula (I) or a solvate, a polymorph, a metabolite, or a pharmaceutically acceptable salt or prodrug thereof, provided that when R₂ and R₂ₐ are taken together, along with the nitrogen atom to which they are attached, form a substituted heteroalicyclyl, wherein the substituted heteroalicyclyl is 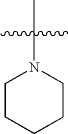
substituted with n-butyl at the para-position, then B cannot be selected from the group consisting of methyl, —C(═O)R₁, and —CH₂OH, wherein R₁ is hydrogen or methyl; or A cannot be methyl. In other embodiments, a pharmaceutical composition comprises a compound of Formula (I) or a solvate, a polymorph, a metabolite, or a pharmaceutically acceptable salt or prodrug thereof, provided that when R₂ and R₂ₐ are taken together, along with the nitrogen atom to which they are attached, form a substituted heteroalicyclyl, wherein the substituted heteroalicyclyl is 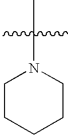
substituted with an alkyl, such as n-butyl, then A, R₃, R₃ₐ, R_{3b}, and R_{3c} cannot all be hydrogen.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990, which is hereby incorporated by reference in its entirety.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternately, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the targeted organ or tissue.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., as disclosed in Remington's Pharmaceutical Sciences, cited above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; and other biocompatible polymers may replace polyethylene glycol, e.g., polyvinyl pyrrolidone. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lacetic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. In some embodiments, the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXAMPLES

Embodiments of the present invention are disclosed in further detail in the following examples, which are not in any way intended to limit the scope of the invention.

General Analytical LC-MS Procedure

Procedure 1 (AP1): The analysis was performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammonium acetate in water, buffer B: 10 mM ammonium acetate in acetonitrile/water 95/5. A gradient was run from 30% B to 100% B in 10 min, dwelling at 100% B for 1 min, and re-equilibrating for 6 min. The system was operated at 1 ml/min.

Procedure 2 (AP2): The analysis was performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammonium acetate in water, buffer B: 10 mM ammonium acetate in acetonitrile/water 95/5. A gradient was run from 30% B to 100% B in 7 min, dwelling at 100% B for 1 min, and re-equilibrating for 5.5 min. The system was operated at 1 ml/min.

Analytical HPLC/S, Ammonium Acetate (AP)

System: Waters/Micromass ZQ2000 LC/MS system consisting of a ZQ single quadropole mass spectrometer equipped with an electrospray ionization interface, and a Waters Alliance HT with a 2795 Separation Module and 996 Photodiode Array Detector.

Column: Reversed phase column (Waters Xterra® MS C₁₈ 3.5 μm, 30×4.6 mm ID) with a guard column cartridge system.

Mobile Phase: A: 10 mM aqueous Ammonium Acetate; B: 10 mM aqueous Ammonium Acetate Acetonitrile/Water (95:5).

Program: 10 min. gradient program starting at 30% B (initial hold for 0.5 min.), over 5 min. to 100% B, hold for 1.5 min., over 0.5 min. to 30% B, hold for 2.5 min. The flow rate was 1 mL/min.

Preparative HPLC/MS, Ammonium Acetate (PP)

System: Waters/Micromass LC/ZMD Autopurification system consisting of a ZMD single quadropole mass spectrometer equipped with an electrospray ionization interface, and a Waters 600E Gradient Pump with in-line degassing, 2700 Sample Manager and 996 Photodiode Array Detector.

Column: Reversed phase column (Waters Xterra® Prep MS C₁₈ 5 μm, 19×100 mm).

Mobile Phase: A: 10 mM aqueous Ammonium Acetate; B: 110 mM aqueous Ammonium Acetate Acetonitrile/Water (95:5).

Program: 12 min. gradient program starting at 30% B (initial hold for 2.5 min.), over 8.5 min. to 100% B, over 0.5 min. to 30% B, hold for 0.5 min. The flow rate was 17 mL/min.

### Typical Procedure 1 (See Scheme 1) (TP1): 1-[1-(3-chloropropyl)-1H-indol-3-yl]-ethanone

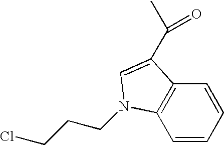

3-Acetylindole (795 mg, 5 mmol), cesium carbonate (3.25 g, 10 mmol) and 1-chloro-3-iodopropane (3.06 g, 15 mmol) were weighed into a MW vial and dry MeCN (15 mL) was added. The vial was capped and heated in the MW at 100° C. for 25 min. The reaction was filtrated and concentrated onto celite and purified by flash chromatography 0-30% EtOAc in heptane. Yield: 971 mg (83%).

¹H NMR (400 MHz, CDCl₃) δ 8.40-8.37 (m, 1H), 7.75 (s, 1H), 7.37-7.28 (m, 3H), 4.36 (t, J=6.0 Hz, 2H), 3.46 (t, J=6.0 Hz, 2H), 2.51 (s, 3H), 2.30 (pentet, J=6.0 Hz, 2H).

### Typical Procedure 2 (See Scheme 1) (TP2): 1-(3-chloropropyl)-7-isopropoxy-1H-indole

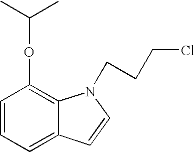

7-isopropoxy-1H-indole (736 mg, 4.2 mmol), cesium carbonate (2.73 g, 8.4 mmol) and 1-chloro-3-iodopropane (2.57 g, 12.6 mmol) were weighed into a vial and dry MeCN (20 mL) was added. The vial was sealed and heated on a shaker at 50° C. for 24 h. The reaction was filtrated and concentrated onto celite and purified by flash chromatography 0-20% EtOAc in heptane. Yield: 750 mg (71%).

¹H NMR (400 MHz, CDCl₃) δ: 7.2 (d, J=7.8 Hz, 1H), 7.04 (d, J=3.1 Hz, 1H), 6.99 (t, J=7.8 Hz, 1H), 6.62 (d, J=7.8 Hz, 1H), 6.43 (d, J=3.1 Hz, 1H), 4.80-4.73 (m, 1H), 4.58-4.49 (m, 2H), 3.49-3.46 (m, 2H), 2.37-2.35 (m, 2H), 1.45 (d, J=5.8 Hz, 6H).

### Typical procedure 3 (See Scheme 2) (TP3): 1-(7-bromo-1-(3-chloropropyl)-1H-indol-3-yl)ethanone

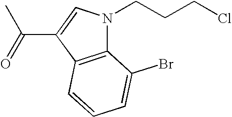

To a solution of 7-bromo-1H-indole (442 mg, 2.25 mmol) in dry CH₂Cl₂ (10 mL) at 0° C. was added Et₂AlCl (3.4 mL, 1.0 M, 3.4 mmol) dropwise. The mixture was stirred at 0° C. for 25 min. A solution of AcCl (0.24 mL, 3.36 mmol) in CH₂Cl₂ (5 mL) was added dropwise and the mixture was stirred at 0° C. until TLC showed complete conversion of the indole (1-3 h). Saturated aqueous NaHCO₃ (10 mL) was added slowly and the mixture was allowed to reach room temperature. The mixture was diluted with CH₂Cl₂ (60 mL) and the mixture was cautiously acidified to pH 4-5 with 2 M HCl (approx. 10 mL) to facilitate phase separation. The aqueous layer was extracted with CH₂Cl₂ (2×10 mL) and the combined organic layers where washed with saturated aqueous NaHCO₃ and evaporated to dryness to give the acetylated compound (496 mg, 93%). This acetylated crude product was dissolved in CH₃CN (10 mL). To this solution was added Cs₂CO₃ (1.55 g, 4.76 mmol) and 1-chloro-3-iodopropane (0.70 mL, 6.52 mmol) and the mixture was stirred at 50° C. overnight. The suspension was diluted with CH₂Cl₂ (70 mL), filtered and adsorbed onto celite. After purification by flash chromatography (heptanes→heptanes:EtOAc 3:2) the title product was obtained (611 mg, 86% over two steps.

¹H NMR (400 MHz, CDCl₃) δ 8.41 (dd, J=8.0, 1.1 Hz, 1H), 7.75 (s, 1H), 7.44 (dd, J=7.7, 1.1 Hz, 1H), 7.12-7.08 (m, 1H), 4.73 (t, J=6.7 Hz, 2H), 3.50-3.47 (m, 2H), 2.50 (s, 3H), 2.39-2.33 (m, 2H).

¹³C NMR (100 MHz, CDCl₃) δ 192.5, 137.7, 132.8, 129.7, 128.7, 123.7, 122.1, 116.5, 103.5, 45.8, 41.3, 34.1, 27.5.

### Typical Procedure (See Scheme 3) (TP4): N-(3-methylbenzyl)-1H-indole-3-carboxamide

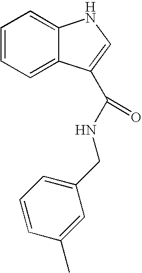

Indole-3-carboxylic acid (644 mg, 4 mmol), 1-hydroxybenzotriazole (810 mg, 6 mmol), EDCl (1.15 g, 6 mmol), TEA (1.82 g, 18 mmol) and 3-methylbenzylamine (485 mg, 4 mmol) were weighed into a MW vial and dry MeCN (10 mL) was added. The vial was capped and heated in the MW at 140° C. for 15 min. The reaction mixture was diluted with EtOAc and washed with water and brine, dried over sodium sulphate, filtered and concentrated in vacuo. The product was purified by recrystallization from MeOH. Yield: 411 mg (39%).

¹H NMR (400 MHz, CDCl₃) δ: 8.67 (bs, 1H), 7.78 (d, J=2.8 Hz, 1H), 7.48 (d, J=8.0 Hz, 1H), 7.25-7.13 (m, 6H), 6.70 (d, J=8.0 Hz, 1H), 6.23 (bt, 1H), 4.68 (d, J=5.6 Hz, 2H), 2.35 (s, 3H).

### Typical Procedure 5 (See Scheme 4) (TP5): 1-(3-chloropropyl)-N-(3,4-dichlorobenzyl)-7-methoxy-1H-indole-3-carboxamide

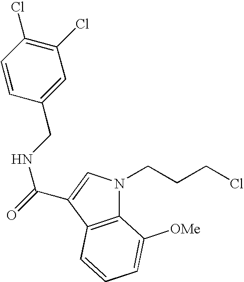

To a stirring suspension of MgI₂ (706 mg, 2.54 mmol) in DCE (4 mL) was added 1,2-dichloro-4-(isocyanatomethyl)benzene (0.39 mL, 2.65 mmol) and 1-(3-chloropropyl)-7-methoxy-[1]-indole (567 mg, 2.53 mmol). The mixture was stirred at 80° C. for 2 h at which point full conversion was observed by TLC (prolonged stirring at room temperature also led to full conversion). The mixture was diluted with EtOAc (200 mL) and the organic layer washed with saturated aqueous NaHCO₃, H₂O, 10% aqueous Na₂SO₃, brine, dried over Na₂SO₄ and evaporated to dryness. After washing the resulting crystals with diethyl ether (3×10 mL) the title compound was obtained as pinkish crystals (788 mg, 73%) which was used without further purification. 0-15% of the corresponding alkyl iodide was occasionally observed in these reactions, in particular when the reaction was performed at 80° C.

¹H NMR (400 MHz, dmso-d₆) δ 8.48-8.45 (m, 1H), 7.93 (s, 1H), 7.75 (dd, J=8.1, 0.8 Hz, 1H), 7.59-7.56 (m, 2H), 7.33 (dd, J=8.3, 2.0 Hz, 1H), 7.04 (t, J=7.9 Hz, 1H), 6.75 (d, J=7.5 Hz, 1H), 4.51-4.43 (m, 4H), 3.90 (s, 3H), 3.60 (t, J=6.3 Hz, 2H), 2.28-2.18 (m, 2H).

¹³C NMR (100 MHz, dmso-d₆) δ 164.1, 146.9, 141.6, 131.8, 130.6, 130.3, 129.1, 129.0, 128.8, 127.5, 125.2, 121.5, 113.8, 109.6, 103.5, 55.4, 46.6, 42.2, 40.9, 34.0.

### Typical Procedure 6 (See Scheme 5) (TP6): 7-isopropoxy-1H-indole

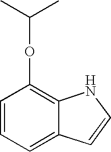

7-hydrozyindol (1.00 g, 7.5 mmol) and resin bound triphenylphosphine (4.25 g, 13 mmol, 3 mmol/g) was taken up in THF (30 mL) and cooled to 0° C., before drop wise addition of diisopropylazodicarboxylate (787 mg, 3.9 mmol). After 40 min at 0° C. isopropanol (1.80 g, 30 mmol) in THF (15 mL) was added slowly. The reaction was left for further 2 h at 0° C., then the cooling was removed and the reaction left at room temperature over night. The reaction mixture was filtered and concentrated onto celite, then purified by flash chromatography 0-20% EtOAc in heptane. Yield: 736 mg (56%).

¹H NMR (400 MHz, CDCl₃) δ: 7.26 (dd, J=0.8 and 7.4 Hz, 1H), 7.17 (t, J=2.8 Hz, 1H), 7.06 (dt, J=0.8 and 7.4 Hz, 1H), 6.60 (d, J=7.4 Hz, 1H), 6.54 (dt, J=0.8 and 2.8 Hz, 1H), 4.76 (hept, J=6.2 Hz, 1H), 1.44 (d, J=6.2 Hz, 6H).

### Typical Procedure 7 (TP7): 3α-(4-chlorophenoxy)-8-azabicyclo[3.2.1]octane

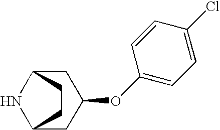

4-Chlorophenol (395 mg, 3 mmol) and resin bound triphenylphosphine (1.20 g, 3.75 mmol, 3 mmol/g) was taken up in THF (10 mL) and cooled to 0° C., before drop wise addition of diisopropylazodicarboxylate (787 mg, 3.9 mmol). After 40 min at 0° C. tert-butyl 3β-hydroxy-8-azabicyclo[3.2.1]octane-8-carboxylate (500 mg, 2.2 mmol) in THF (5 mL) was added slowly. The reaction was continued for 2 h at 0° C., then the cooling was removed and the reaction left at room temperature over night. The reaction mixture was filtered and concentrated onto celite, then purified by flash chromatography 0-30% EtOAc in heptane. The product was taken up in DCM (5 mL), TFA (5 mL) was added and the reaction mixture left stirring for 1 h and concentrated in vacuo. The product was taken up in EtOAc, then washed with NaOH (2 N), dried over sodium sulphate, filtered and concentrated in vacuo. Yield. 370 mg (71% over two steps).

¹H NMR (400 MHz, CD₃OD) δ: 7.29-7.26 (m, 2H), 6.90-6.88 (m, 2H), 4.67 (bt, J=4.4 Hz, 1H), 3.92-3.91 (m, 2H), 2.39-2.00 (m, 7H).

¹³C NMR (100 MHz, CD₃OD) δ: 159.7, 133.5, 129.9, 120.8, 72.4, 58.0, 37.0, 30.2.

### Typical Procedure 8 (See Scheme 6) (TP8): 1-(7-Methoxy-1H-indol-3-yl)ethanone

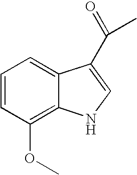

MeMgBr (3 ml, 3M in ether, 9 mmol) was added to 7-methoxy-1H-indole dissolved in dry CH₂Cl₂ (6 ml) at 0-5° C. The resulting red solution was stirred for 1 h at room temperature. Freshly distilled acetyl chloride (353 mg, 4.5 mmol) was then added at 0-5° C. and the resulting brown solution was stirred for 1 h at room temperature. Aqueous HCl (2M) was added to the reaction mixture and the organic phase was separated. The aqueous phase was extracted with CH₂Cl₂ and the combined organic phases were washed with water, brine and dried with Na₂SO₄. The filtrate was concentrated at reduced pressure and the crude product was purified by crystallization (heptane/ethyl acetate 3:1), which gave 345 mg (70%) of the title compound as brown crystals.

¹H NMR (400 MHz, CDCl₃) δ 8.80 (br s, 1H), 7.93 (d, 1H, J=8.0 Hz), 7.81 (d, 1H, J=2.9 Hz), 7.20 (t, 1H, J=8 Hz), 6.73 (d, 1H J=7.9 Hz), 3.96 (s, 3H), 2.55 (s, 3H).

### Typical Procedure 9 (See Scheme 7) (TP9): (1S,4S)-2-(2-(4-fluorophenoxy)ethyl)-2,5-diazabicyclo[2.2.2]octane

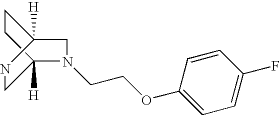

A 4 mL disposable vial was charged with (1S,4S)-tert-butyl 2,5-diazabicyclo[2.2.2]octane-2-carboxylate hydrochloride (252 mg, 1.02 mmol), 1-(2-bromoethoxy)-4-fluorobenzene (362 mg, 1.65 mmol), Cs₂CO₃ (576 mg, 1.77 mmol) and CH₃CN (2 mL). The mixture was sealed and stirred at 60 CC overnight. The suspension was diluted with CH₂Cl₂ (25 mL), filtered and evaporated to dryness. The resulting crude product was dissolved in CH₂Cl₂ (10 mL) and TFA (5 mL) was cautiously added. After stirring at room temperature for 3 h the mixture was evaporated to dryness. The crude product was dissolved in a minimal amount of MeOH and purified by solid phase extraction using a SCX cartridge eluding with NH₃(MeOH) to give the title compound (191 mg, 75% over two steps).

¹H NMR (400 MHz, CDCl₃) δ 6.98-6.93 (m, 2H), 6.85-6.82 (m, 2H), 4.04-4.00 (m, 2H), 3.44-3.41 (m, 1H), 3.10-3.08 (m, 1H), 3.00-2.97 (m, 4H), 2.73-2.70 (m, 3H), 2.10-2.02 (m, 1H), 1.95-1.88 (m, 1H), 1.80-1.70 (m, 2H).

¹³C NMR (100 MHz, CDCl₃) δ 157.5 (d, J=237 Hz), 155.2, 116.0 (d, J=29 Hz), 115.9 (d, J=1.4 Hz), 67.9, 57.8, 55.5, 50.2, 45.6, 45.0, 25.8, 24.2.

### Typical Procedure 10 (See Scheme 8) (TP10): Tert-butyl (1R,5S)-3α-(2-oxo-2-phenylethyl)-8-azabicyclo[3.2.1]octane-8-carboxylate

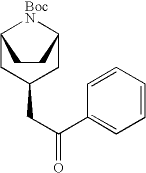

To a solution of [(1R,5S)-8-(tert-butoxycarbonyl)-8-azabicyclo[3.2.1]oct-3α-yl]acetic acid (807 mg, 3 mmol) in THF (10 mL) was added CDMT (624 mg, 3.6 mmol) and NMM (0.99 mL, 9 mmol). A white precipitate was formed during stirring for 1 hour, then N,O-dimethylhydroxylamine hydrochloride (291 mg, 3 mmol) was added and the reaction mixture left stirring for 16 h, quenched with water (20 mL) and extracted with ether. The ether phase washed with sat. aq. Na₂CO₃ and HCl (1 N), then dried over Na₂SO₄ and concentrated in vacuo. Yield: 894 mg (95%).

The crude product was taken up in THF and cooled to 0° C., then phenyl grignard (3 mL, 2 M, 6 mmol) was added and the ice bath removed. After 1 h at rt the reaction mixture was quenched with sat. aq. NH₄Cl. The product was extracted with EtOAc and the organic phase washed with Na₂CO₃, then HCl (1 N), dried over Na₂SO₄ and concentrated in vacuo. Crude yield: 847 mg (90%). UV/MS: 84/62.

¹H NMR (CDCl₃) δ: 7.93-7.90 (m, 2H), 7.58-7.55 (m, 1H), 7.47-7.43 (m, 2H), 4.24-4.12 (m, 2H), 3.12-3.11 (m, 2H), 2.04-2.00 (m, 1H), 1.73-1.71 (m, 2H), 1.44 (s, 9H), 1.30-1.26 (m, 4H), 0.89-0.86 (m, 2H).

### Typical Procedure 11 (See Scheme 9) (TP11): (1R,5S)-3α-(2-phenylethyl)-8-azabicyclo[3.2.1]octane

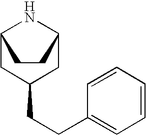

Tert-butyl (1R,5S)-3α-(2-oxo-2-phenylethyl)-8-azabicyclo[3.2.1]octane-8-carboxylate (847 mg, 2.5 mmol) and KOH (540 mg, 9.6 mmol) was taken up in diethyleneglycol (5 mL) then hydrazine monohydrate (401 μL, 8.3 mmol) was added and the RM was heated to 140° C. on an oil bath for 16 h. Cooled on an ice bath and quenched with HCl (2 N). Extracted with EtOAc, then dried over Na₂SO₄ and concentrated in vacuo onto celite. The crude product was purified by flash chromatography 0-20% EtOAc in heptane. Yield: 234 mg (30%). The isolated product was taken up in a mixture of TFA (2 mL) and DCM (2 mL) and stirred for 1 h at rt, concentrated in vacuo, then taken up in EtOAc, washed with NaOH (2 N), dried over Na₂SO₄ and concentrated in vacuo. Yield: 160 mg (96%).

¹H NMR (400 MHZ, CDCl₃): δ 7.30-7.14 (m, 5H), 3.89-3.83 (m, 2H), 2.63-2.59 (m, 2H), 2.33-2.26 (m, 2H), 2.13-2.10 (m, 2H), 1.91-1.68 (m, 5H), 1.59-1.55 (m, 2H).

¹³C NMR (100 MHz, CDCl₃) major isomer: δ 142.0, 128.7, 128.5, 126.2, 54.3, 39.7, 35.0, 33.6, 27.5, 27.4.

### Typical Procedure 12 (See Scheme 10) (TP12): 1-(1-(3-(4-(4-Fluorophenoxy)piperidin 1-yl)propyl)-7-methoxy-1H-indol-3-yl)ethanone oxalate C658b

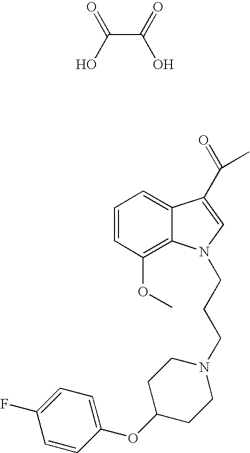

4-(4-fluorophenoxy)piperidine hydrochloride (163 mg, 0.70 mmol) was added to 1-(1-(3-Chloropropyl)-7-methoxy-1H-indol-3-yl)ethanone (93 mg, 0.35 mmol), triethylamine (99 μl, 0.70 mmol) and NaI (cat) in dry DMF (4 ml). The reaction mixture was shaken at 80° C. for 15 h and at 120° C. for 1 h. Ethyl acetate and water was then added to the reaction mixture and the organic phase washed repeatedly with water and finally with brine. Celite was added to the filtrate and the volatile material was removed at reduced pressure. The crude product on celite was purified by column chromatography (heptane/ethyl acetate 1:1 to 100% ethyl acetate and then ethyl acetate/methanol 9:1 to 4:1), which gave 90 mg (61%) of 1-(1-(3-(4-(4-fluorophenoxy)piperidin-1-yl)propyl 7-methoxy-1H-indol-3-yl)ethanone compound as a clear oil.

¹H NMR (400 MHz, CDCl₃) δ 7.97 (d, 1H, J=8.0 Hz), 7.70 (s, 1H), 7.17 (t, 1H, J=8 Hz), 6.95 (m, 2H), 6.84 (m, 2H), 6.71 (d, 1H J=7.8 Hz), 4.47 (t, 2H, J=6.4 Hz), 4.24 (m, 1H), 3.94 (s, 3H), 2.73 (m, 1H), 2.49 (s, 3H), 2.32 (m, 4H), 2.05 (m, 4H), 1.84 (m, 2H). Oxalic acid (1.1 eq) dissolved in acetone (1 ml) was added to the clear oil dissolved in acetone (1 ml). The precipitant was filtered off and dried to yield 90 mg of the title compound as white crystals. MS (ES⁺, M+1)=425.

### Typical Procedure 13 (See Scheme 11) (TP13): N-methoxy-N-methyl-1H-indole-3-carboxamide

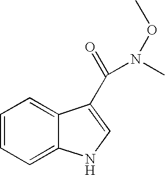

To a solution of 1H-indole-3-carboxylic acid (650 mg, 4 mmol) in DCM (10 mL) in a MW vial was added N,O-dimethylhydroxylamine hydrochloride (423 mg, 4.4 mmol), PPh₃ (800 mg, 8 mmol), CCl₄ (4 mL) and MMP (0.98 mL, 8.9 mmol). The vial was capped and heated in the MW at 100° C. for 12 min, the reaction mixture was subsequently stirred at rt over night. The resulting mixture was filtered, diluted with DCM, washed with HCl (0.5 N) and sat. aq. Na₂CO₃ then dried over Na₂SO₄ and concentrated in vacuo. Yield: 219 mg (27%).

¹H NMR (400 MHZ, CDCl₃): δ 8.85 (bs, 1H), 8.4 (d, J=7.6 Hz, 1H), 7.97 (d, J=2.1 Hz, 1H), 7.40.7.22 (m, 3H), 3.71 (s, 3H), 3.42 (s, 3H).

### 1-(1H-pyrrolo[2,3-b]pyridin-3-yl)ethanone

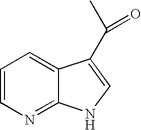

To a suspension of AlCl₃ (4.91 g, 36.8 mmol) in CH₂Cl₂ (100 mL) at rt was added 1H-pyrrolo[2,3-b]pyridine (899 mg, 7.61 mmol). After 60 min AcCl (2.7 mL, 37.8 mmol) was added dropwise and the mixture was stirred overnight. The reaction was quenched by careful addition of MeOH (35 mL) and evaporated to dryness. Saturated aqueous NaRCO₃ (150 mL) and EtOAc (100 mL) were added to the residue followed by vigorous stirring. The aqueous layer was extracted with EtOAc (2×100 mL), and the combined organic layers were dried over Na₂SO₄ and evaporated to dryness to give the title compound as colorless crystals (803 mg, 66%).

¹H NMR (400 MHz, CD₃OD) δ 8.70 (dd, J=7.9, 1.6 Hz, 1H), 8.37-8.35 (m, 2H), 7.37 (dd, J=7.9, 5.0 Hz, 1H), 2.58 (s, 3H).

### 7-methoxy-1H-indole-3-carbonitrile

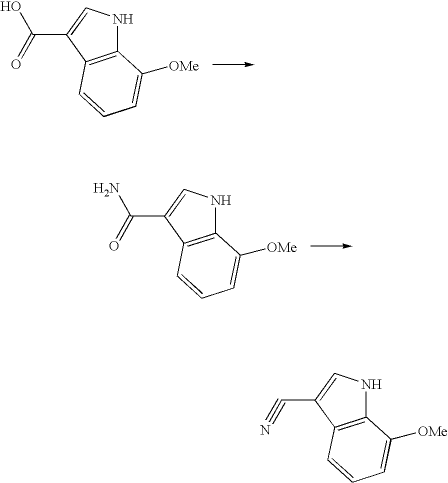

To a solution of 7-methoxy-1H-indole-3-carboxylic acid (363 mg, 1.90 mmol) in DMF (10 mL) was added carbonyl diimidazole (382 mg, 2.36 mmol) in one portion. After stirring at room temperature for 1 h 30 min 28% aqueous NH₃ (0.50 mL, approx. 7 mmol) was added, and the mixture was left stirring overnight. After evaporation to dryness the remanens was taken up in EtOAc (200 mL) and the organic layer washed with H₂O (2×10 mL), saturated aqueous NaHCO₃ (10 mL), brine, dried over Na₂SO₄ and evaporated to dryness to give the intermediate amide (527 mg). This amide was dissolved in a mixture of CH₃CN (15 mL) and H₂O (15 mL). The mixture was heated to 50° C. and Pd(OAc)₂ (408 mg, 1.81 mmol) was added in portions over 24 h. The black reaction mixture was quenched with saturated aqueous NaHCO₃ (25 mL) and the CH₃CN was removed by rotary evaporation. The remaining aqueous layer was extracted with EtOAc (3×25 mL). The combined organic layers washed with brine, dried over Na₂SO₄ and evaporated to dryness to give the pure title compound (134 mg, 41% overall yield.).

One peak GC-MS m/z (relative intensity) 172(100), 157(67), 129(60), 102(11).

### 1-(1-(3-chloropropyl)-1H-pyrrolo[2,3-b]pyridin-3-yl)-2,2,2-trifluoroethanone

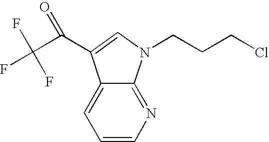

Prepared according to TP2, see also Scheme 1, using 2,2,2-trifluoro-1-(1H-pyrrolo[2,3-b]pyridin-3-yl)ethanone (253 mg, 1.18 mmol), Cs₂CO₃ (810 mg, 2.49 mmol) and 1-chloro-3-iodopropane (0.40 mL, 3.7 mmol) in CH₃CN (10 mL). Purification was by flash chromatography (heptanes→heptanes:EtOAc 3:2) to give the title compound (241 mg, 70%).

¹H NMR (400 MHz, CDCl₃) δ 8.59 (dd, J=7.9, 1.6 Hz, 1H), 8.43 (dd, J=4.7, 1.6 Hz, 1H), 8.11 (d, J=1.7 Hz, 1H), 7.29 (dd, J=7.9, 4.7 Hz, 1H), 4.58-4.54 (m, 2H), 3.53-3.50 (m, 2H), 2.46-2.39 (m, 2H).

¹³C NMR (100 MHz, CDCl₃) δ 174.9 (q, J=35.4 Hz), 147.8, 145.6, 137.5 (q, J=4.8 Hz), 130.9, 119.7, 119.4, 116.8 (q, J=290.8 Hz), 108.0, 43.2, 41.3, 31.8.

### 1-(1-(3-chloropropyl)-1H-pyrrolo[2,3-b]pyridin-3-yl)ethanone

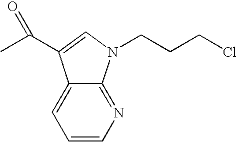

Prepared according to TP2, see also Scheme 1, by using 1-(1H-pyrrolo[2,3-b]pyridin-3-yl)ethanone (492 mg, 3.07 mmol), Cs₂CO₃ (1.53 g, 4.70 mmol), 1-chloro-3-iodopropane (0.90 mL, 8.4 mmol) in CH₃CN (15 mL) to give the title compound (338 mg, 47%).

¹H NMR (400 MHz, CDCl₃) δ 8.51 (dd, J=8.0, 1.6 Hz, 1H), 8.29 (dd, J=4.8, 1.6 Hz, 1H), 7.82 (s, 1H), 7.14 (dd, J=8.0, 4.8 Hz, 1H), 4.44-4.40 (m, 2H), 3.45-3.42 (m, 2H), 2.43 (s, 3H), 2.35-2.28 (m, 2H).

¹³C NMR (100 MHz, CDCl₃) δ 192.4, 147.6, 144.2, 134.5, 130.7, 118.5, 118.3, 115.2, 42.3, 41.4, 31.9, 26.9.

### 1-(3-chloropropyl)-7-methoxy-1H-indole-3-carbonitrile

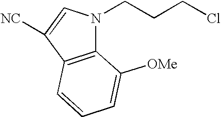

Prepared according to TP2, see also Scheme 1, by using 7-methoxy-1H-indole-3-carbonitrile (134 mg, 0.78 mmol), Cs₂CO₃ (809 mg, 2.48 mmol), 1-chloro-3-iodopropane (0.45 mL, 4.2 mmol) in CH₃CN (6 mL) to give the title compound (138 mg, 71%).

¹H NMR (400 MHz, CDCl₃) δ 7.51 (s, 1H), 7.32-7.30 (m, 1H), 7.18-7.14 (m, 1H), 6.73-6.71 (m, 1H), 4.57-4.54 (m, 2H), 3.94 (s, 3H), 3.45-3.42 (m, 2H), 2.31-2.25 (m, 2H).

¹³C NMR (100 MHz, CDCl₃) δ 147.6, 135.7, 130.3, 124.7, 122.9, 115.6, 112.2, 104.3, 85.7, 55.4, 47.1, 41.3, 33.8.

### 1-(3-chloropropyl)-N-(3-methylbenzyl)-1H-indole-3-carboxamide

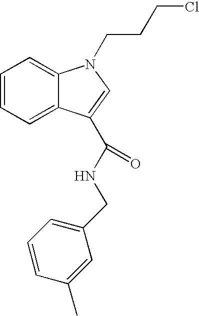

Prepared according to TP1, see also Scheme 1, using N-(3-methylbenzyl)-1H-indole-3-carboxamide (264 mg, 1 mmol), cesium carbonate (650 mg, 2 mmol) and 1-chloro-3-iodopropane (612 mg, 3 mmol) in MeCN (4 mL). The product was purified by flash chromatography 0-30% EtOAc in heptane. Yield: 280 mg (82%).

¹H NMR (400 MHz, CDCl₃) δ: 7.60 (s, 1H), 7.54 (dd, J=8.0 and 0.8 Hz, 1H), 7.26-7.10 (m, 6H), 6.68 (d, J=8.0 Hz, 1H), 6.15 (bt, 1H), 4.66 (d, J=5.6 Hz, 2H), 4.56 (t, J=6.6 Hz, 2H), 3.47 (t, J=6.6 Hz, 2H), 2.35 (s, 3H), 2.29 (pentet, J=6.6 Hz, 2H).

### N-(3-chlorobenzyl)-1H-indole-3-carboxamide

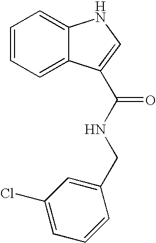

Prepared according to TP4, see also Scheme 3, using indole-3-carboxylic acid (644 mg, 4 mmol), 1-hydroxybenzotriazole (810 mg, 6 mmol), EDCl (1.15 g, 6 mmol), TEA (1.82 g, 18 mmol) and 3-chlorobenzylamine (566 mg, 4 mmol) in MeCN (10 mL). The product was purified by recrystallization from MeOH. Yield: 540 mg (48%).

¹H NMR (400 MHz, CDCl₃) δ: 8.58 (bs, 1H), 7.96-7.94 (m, 1H), 7.82 (d, J=2.4 Hz, 1H), 7.45-7.44 (m, 1H), 7.39 (s, 1H), 7.30-7.26 (m, 5H), 6.26 (bt, 1H), 4.70 (d, J=5.6 Hz, 2H).

### 1-(3-chloropropyl)-N-(3-chlorobenzyl)-1H-indole-3-carboxamide

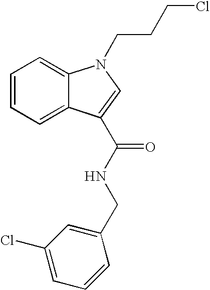

Prepared according to TP4, see also Scheme 3, using N-(3-chlorobenzyl)-1H-indole-3-carboxamide (284 mg, 1 mmol), cesium carbonate (650 mg, 2 mmol) and 1-chloro-3-iodopropane (612 mg, 3 mmol) in MeCN (4 mL). The product was purified by flash chromatography 0-30% EtOAc in heptane. Yield: 170 mg (48%).

¹H NMR (400 MHz, CDCl₃) δ: 7.98-7.96 (m, 1H), 7.77-7.72 (m, 1H), 7.44-7.24 (m, 7H), 6.28 (bt, 1H), 4.68 (d, J=6.0 Hz, 2H), 4.36 (t, J=6.4 Hz, 2H), 3.47 (t, J=6.4 Hz, 2H), 2.30 (pentet, J=6.4 Hz, 2H).

### 7-methoxy-N-(3-methylbenzyl)-1H-indole-3-carboxamide

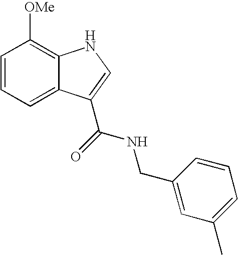

Prepared according to TP4, see also Scheme 3, using 7-methoxyindole-3-carboxylic acid (764 mg, 4 mmol), 1-hydroxybenzotriazole (810 mg, 6 mmol), EDCl (1.15 g, 6 mmol), TEA (1.82 g, 18 mmol) and 3-methylbenzylamine (485 mg, 4 mmol) in MeCN (10 mL). The product was purified by recrystallization from MeOH. Yield: 466 mg (40%).

¹H NMR (400 MHz, CDCl₃) δ: 7.81-7.79 (m, 1H), 7.49 (d, J=6.4 Hz, 1H), 7.33-7.10 (m, 5H), 6.72 (d, J=6.4 Hz, 1H), 4.70-4.68 (m, 2H), 3.97 (s, 3H), 2.38 (s, 3H).

### N-(3-chlorobenzyl)-7-methoxy-1H-indole-3-carboxamide

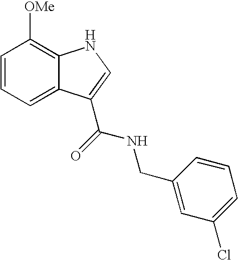

Prepared according to TP4, see also Scheme 3, from 7-methoxyindole-3-carboxylic acid (764 mg, 4 mmol) and 3-chlorobenzylamine (564 mg, 4 mmol) to yield the title compound. Yield: 478 mg (38%).

¹H NMR (400 MHz, CDCl₃) δ: 7.78 (d, J=2.7 Hz, 1H), 7.47 (d, J=8.2 Hz, 1H), 7.28-7.20 (m, 4H), 7.16 (t, J=8.2 Hz, 1H), 6.70 (d, J=7.4 Hz, 1H), 4.68 (d, J=5.8 Hz, 2H), 3.96 (s, 3H).

### N-isobutyl-7-methoxy-1H-indole-3-carboxamide

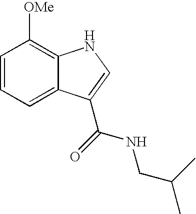

Prepared according to TP4, see also Scheme 3, from 7-methoxyindole-3-carboxylic acid (764 mg, 4 mmol) and isobutyl amine (292 mg, 4 mmol) to yield the title compound. Yield: 422 mg (43%).

¹H NMR (400 MHz, CDCl₃) δ: 7.77 (d, J=2.7 Hz, 1H), 7.45 (d, J=7.8 Hz, 1H), 7.17 (t, J=7.8 Hz, 1H), 6.70 (d, J=7.8 Hz, 1H), 3.96 (s, 3H), 3.34 (t, J=6.6 Hz, 2H), 1.95-1.92 (m, 1H), 1.02-1.00 (m, 6H).

¹³C NMR (100 MHz, CDCl₃) δ: 163.5, 146.7, 127.7, 126.1, 122.4, 114.7, 112.3, 102.8, 102.3, 55.6, 47.1, 29.0, 20.5.

### 1-(3-chloropropyl)-7-methoxy-N-(3-methylbenzyl)-1H-indole-3-carboxamide

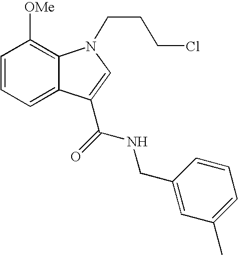

Prepared according to TP2, see also Scheme 1, using 7-methoxy-N-(3-methylbenzyl)-1H-indole-3-carboxamide (441 mg, 1.5 mmol), cesium carbonate (975 mg, 3 mmol) and 1-chloro-3-iodopropane (918 mg, 4.5 mmol) in MeCN (10 mL) was added. Yield: 438 mg (78%).

¹H NMR (400 MHz, CDCl₃) δ: 7.60 (s, 1H), 7.54 (dd, J=0.8 and 7.8 Hz, 1H), 7.26-7.10 (m, 5H), 6.68 (d, J=7.8 Hz, 1H), 4.65 (d, J=5.6 Hz, 2H), 4.56 (t, J=6.6 Hz, 2H), 3.94 (s, 3H), 3.47 (t, J=6.4 Hz, 2H), 2.35 (s, 3H), 2.31-2.27 (m, 2H).

¹³C NMR (100 MHz, CDCl₃) δ: 173.1, 148.2, 139.2, 132.6, 128.8, 128.3, 127.8, 126.3, 125.0, 122.4, 113.1, 113.1, 111.8, 111.2, 103.5, 55.5, 47.2, 43.7, 41.9, 34.5, 34.4.

### N-(3-chlorobenzyl)-1-(3-chloropropyl)-7-methoxy-1H-indole-3-carboxamide

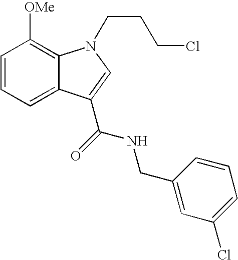

Prepared according to TP2, see also Scheme 1, from N-(3-chlorobenzyl)-7-methoxy-1H-indole-3-carboxamide (471 mg, 1.5 mmol) to yield the title compound. Yield: 418 mg (72%).

¹H-NMR (400 MHz, CDCl₃) δ: 7.61 (s, 1H), 7.53 (d, J=8.2 Hz, 1H), 7.28-7.24 (m, 4H), 7.14 (t, J=7.8 Hz, 1H), 6.69 (d, J=7.8 Hz, 1H), 4.66 (d, J=5.8 Hz, 2H), 4.56 (t, J=6.7 Hz, 2H), 3.95 (s, 3H), 3.47 (t, J=6.7 Hz, 2H), 2.29 (pent, J=6.7 Hz, 2H).

¹³C-NMR (100 MHz, CDCl₃) δ: 165.2, 147.9, 141.3, 132.7, 130.2, 128.3, 128.0, 127.8, 126.2, 126.1, 122.6, 113.2, 113.1, 111.0, 103.7, 76.7, 55.6, 47.2, 43.1, 41.9, 34.5.

### 1-(3-chloropropyl)-N-isobutyl-7-methoxy-1H-indole-3-carboxamide

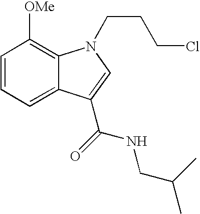

Prepared according to TP2, see also Scheme 1, from N-isobutyl-7-methoxy-1H-indole-3-carboxamide (369 mg, 1.5 mmol) to yield the title compound. Yield: 342 mg (70%).

¹H-NMR (400 MHz, CDCl₃) δ: 7.61 (s, 1H), 7.55 (d, J=7.8 Hz, 1H), 7.14 (t, J=7.8 Hz, 1H), 6.69 (d, J=7.8 Hz, 1H), 4.56 (t, J=6.4 Hz, 2H), 3.95 (s, 3H), 3.47 (t, J=5.8 Hz, 2H), 3.32 (t, J=5.8 Hz, 2H), 2.30-2.27 (m, 2H), 1.93 (hept, J=6.4 Hz, 1H), 1.00 (d, J=6.4 Hz, 6H).

### 1-(3-chloropropyl)-7-isopropoxy-N-(3-methylbenzyl)-1H-indole-3-carboxamide

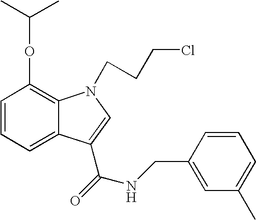

Prepared according to TP5 from 1-(3-chloropropyl)-7-isopropoxy-1H-indole (251 mg, 1 mmol) and 1-(isocyanatomethyl)-3-methylbenzene (154 mg, 1.05 mmol). The obtained solid washed with MeOH. Yield 269 mg (67%).

¹H NMR (400 MHz, CDCl₃) δ: 7.59 (t, J=7.6 Hz, 1H), 7.51-7.46 (m, 1H), 7.26-7.17 (m, 3H), 7.11-7.06 (m, 2H), 6.66 (d, J=7.8 Hz, 1H), 4.75 (hept, J=6.3 Hz, 1H), 4.65 (d, J=5.5 Hz, 2H), 4.57 (t, J=6.7 Hz, 2H), 3.48 (1, J=6.3 Hz, 2H), 2.30 (pent, J=6.2 Hz, 2H), 1.56 (s, 3H), 1.42 (d, J=5.5 Hz, 6H).

### 1-(3-chloropropyl)-N-(3,4-dichlorobenzyl)-7-isopropoxy-1H-indole-3-carboxamide

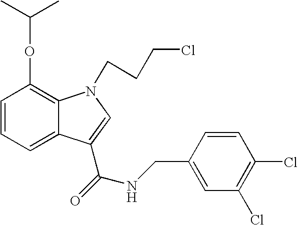

Prepared according to TP5, see also Scheme 4, from 1-(3-chloropropyl)-7-isopropoxy-1H-indole (251 mg, 1 mmol) and 1,2-dichloro-4-(isocyanatomethyl)benzene (212 mg, 1.05 mmol). The obtained solid washed with MeOH. Yield 270 mg (61%).

¹H-NMR (400 MHz, CDCl₃) δ: 7.64-7.60 (m, 1H), 7.49-7.38 (m, 3H), 7.26-7.21 (m, 1H), 7.11-7.09 (m, 1H), 6.69-6.66 (m, 1H), 4.76-4.49 (m, 4H), 3.49-3.46 (m, 1H), 2.32-2.29 (m, 2H), 1.57-1.56 (m, 2H), 1.43-1.39 (m, 6H).

### 1-(1-(3-chloropropyl)-7-methyl-1H-indol-3-yl)ethanone

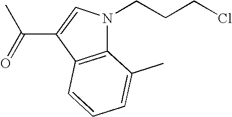

Acetylation was carried out according to TP8, see also Scheme 6, using 7-methyl-1H-indole (369 mg, 2.81 mmol), MeMgBr (2.8 mL, 1M, 2.8 mmol) in CH₂Cl₂ (6 mL). Subsequent alkylation of the crude product was carried out according to TP1 using 3-iodo-1-chloropropane (0.45 mL, 4.2 mmol) and Cs₂CO₃ (1.19 g, 3.7 mmol) in a mixture of CH₃CN (3 mL) and DMF (2 mL) to give the title compound. Yield: 248 mg (35%).

¹H NMR (400 MHz, CDCl₃) δ 8.31-8.29 (m, 1H), 7.73 (s, 1H), 7.19-7.15 (m, 1H), 7.04-7.02 (m, 1H), 4.57 (t, J=6.7 Hz, 2H), 3.52-3.50 (m, 2H), 2.72 (s, 3H), 2.52 (s, 3H), 2.32-2.25 (m, 2H).

### (1-(3-chloropropyl)-7-methoxy-1H-indol-3-yl)(cyclopropyl)methanone

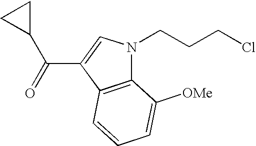

Prepared according to TP3, see also Scheme 2, by using 7-methoxy-1H-indole (365 mg, 2.48 mmol), Et₂AlCl (3.5 mL, 1.0 M, 3.5 mmol), cyclopropanecarbonyl chloride (0.31 mL, 3.4 mmol) in CH₂Cl₂ (6 mL) and Cs₂CO₃ (2.38 g, 7.2 mmol), 1-chloro-3-iodopropane (0.70 mL, 6.52 mmol) in CH₃CN (10 mL) to give the title compound (401 mg, 55% over two steps).

¹H NMR (400 MHz, CDCl₃) δ 8.01 (dd, J=8.1, 0.9 Hz, 1H), 7.79 (s, 1H), 7.13-7.12 (m, 1H), 6.70 (d, J=7.8 Hz, 1H), 4.56 (t, J=6.5 Hz, 2H), 3.92 (s, 3H), 3.48-3.45 (m, 2H), 2.44-2.38 (m, 1H), 2.33-2.27 (m, 2H), 1.23-1.20 (m, 2H), 0.93-0.89 (m, 2H).

¹³C NMR (100 MHz, CDCl₃) δ 194.7, 146.9, 135.4, 128.7, 125.8, 123.0, 117.1, 115.0, 104.0, 55.1, 47.0, 41.6, 33.8, 17.9, 9.6.

### 1-(7-chloro-1-(3-chloropropyl)-1H-indol-3-yl)ethanone

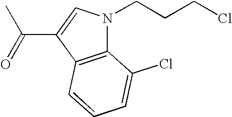

Prepared according to TP3, see also Scheme 2, by using 7-chloro-1H-indole (310 mg, 2.04 mmol), Et₂AlCl (3.0 mL, 1.0 M, 3.0 mmol), AcCl (0.21 mL, 2.9 mmol) in CH₂Cl₂ (9 mL) and Cs₂CO₃ (1.32 g, 4.1 mmol), 1-chloro-3-iodopropane (0.65 mL, 6.1 mmol) in CH₃CN (10 mL) to give the title compound (438 mg, 79% over two steps).

¹H NMR (400 MHz, CDCl₃) δ 8.32 (dd, J=7.9, 1.2 Hz, 1H), 7.69 (s, 1H), 7.21-7.11 (m, 2H), 4.63 (t, J=6.7 Hz, 2H), 3.45 (t, J=5.9 Hz, 2H), 2.46 (s, 3H), 2.34-2.28 (m, 2H).

### (1-(3-chloropropyl)-7-methoxy-1H-indol-3-yl)(phenyl)methanone

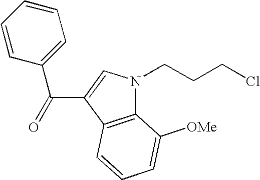

Prepared according to TP3, see also Scheme 2, by using 7-methoxy-1H-indole (318 mg, 2.16 mmol), Et₂AlCl (3.2 mL, 1.0 M, 3.2 mmol), benzoyl chloride (0.39 mL, 3.4 mmol) in CH₂Cl₂ (6 mL) and Cs₂CO₃ (1.67 g, 5.1 mmol), 1-chloro-3-iodopropane (0.70 mL, 6.5 mmol) in CH₃CN (10 mL) to give the title compound (430 mg, 61% over two steps). ¹H NMR (400 MHz, CDCl₃) δ 8.04 (dd, J=8.1, 0.9 Hz, 1H), 7.82-7.80 (m, 2H), 7.56-7.46 (m, 4H), 7.25-7.21 (m, 1H), 6.77 (d, J=7.9 Hz, 1H), 4.57 (t, J=6.6 Hz, 2H), 3.96 (s, 3H), 3.48-3.45 (m, 2H), 2.34-2.28 (m, 2H).

### 1-(7-bromo-1-(3-chloropropyl)-2-methyl-1H-indol-3-yl)ethanone

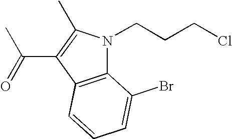

Prepared according to TP3, see also Scheme 2, by using 7-bromo-2-methyl-1 h-indole (425 mg, 2.02 mmol), Et₂AlCl (3.0 mL, 1.0 M, 3.0 mmol), AcCl (0.21 mL, 2.9 mmol) in CH₂Cl₂ (10 mL) and Cs₂CO₃ (1.61 g, 4.94 mmol), 1-chloro-3-iodopropane (0.68 mL, 6.3 mmol) in CH₃CN (10 mL) to give the title compound (449 mg, 68% over two steps).

¹H NMR (400 MHz, CDCl₃) δ 7.98 (dd, J=8.1, 1.0 Hz, 1H), 7.36 (dd, J=7.7, 1.0 Hz, 1H), 7.02 (dd, J=8.1, 7.7 Hz, 1H), 4.66-4.63 (m, 2H), 3.59 (t, J=6.1 Hz, 2H), 2.72 (s, 3H), 2.60 (s, 3H), 2.24-2.17 (m, 2H).

¹³C NMR (100 MHz, CDCl₃) δ 194.0, 145.4, 131.9, 129.7, 127.9, 122.6, 120.1, 114.9, 103.2, 41.4, 41.3, 33.8, 31.7, 12.5.

### 1-(1-(3-chloropropyl)-7-ethyl-1H-indol-3-yl)ethanone

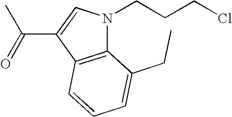

Prepared according to TP3, see also Scheme 2, by using 7-ethyl-1H-indole (307 mg, 2.11 mmol), Et₂AlCl (3.2 mL, 1.0 M, 3.2 mmol), AcCl (0.23 mL, 3.2 mmol) in CH₂Cl₂ (10 mL) and Cs₂CO₃ (1.38 g, 4.26 mmol), 1-chloro-3-iodopropane (0.68 mL, 6.3 mmol) in CH₃CN (10 mL) to give the title compound (334 mg, 60% over two steps).

¹H NMR (400 MHz, CDCl₃) δ 8.34 (dd, J=8.0, 1.3 Hz, 1H), 7.73 (s, 1H), 7.23-7.19 (m, 1H), 7.10-7.08 (m, 1H), 4.49 (t, J=6.8 Hz, 2H), 3.49-3.46 (m, 2H), 3.04-2.98 (m, 2H), 2.49 (s, 3H), 2.27-2.21 (m, 2H), 1.34 (t, J=7.5 Hz, 3H).

¹³C NMR (100 MHz, CDCl₃) δ 192.6, 136.7, 134.1, 127.7, 127.3, 124.4, 122.7, 120.4, 116.8, 46.2, 41.2, 33.8, 27.3, 25.1, 15.8.

### 1-(1-(3-chloro-2-methyl)propyl)-7-methoxy-1H-indol-3-yl)ethanone

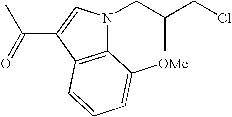

Prepared according to TP3, see also Scheme 2, by using 7-methoxy-1H-indole (669 mg, 4.55 mmol), Et₂AlCl (6.8 mL, 1.0 M, 6.8 mmol), AcCl (0.49 mL, 6.8 mmol) in CH₂Cl₂ (15 mL) and Cs₂CO₃ (1.99 g, 6.13 mmol), 1-chloro-3-iodo-2-methylpropane (0.94 mL, 8.0 mmol) in CH₃CN (20 mL) to give the title compound (589 mg, 70% over two steps).

¹H NMR (400 MHz, CDCl₃) δ 8.00 (dd, J=8.1, 0.9 Hz, 1H), 7.61 (s, 1H), 7.17-7.12 (m, 1H), 6.69-6.67 (m, 1H), 4.38-4.24 (m, 2H), 3.90 (s, 3H), 3.46-3.34 (m, 2H), 2.51-2.46 (m, 4H), 1.02 (d, J=6.8 Hz, 3H).

¹³C NMR (100 MHz, CDCl₃) δ 192.6, 146.9, 136.1, 128.6, 126.0, 123.0, 116.5, 114.8, 104.0, 55.1, 52.5, 47.8, 36.7, 27.3, 15.0.

### 1-(1-(3-chloropropyl)-7-methoxy-1H-indol-3-yl)-2-phenylethanone

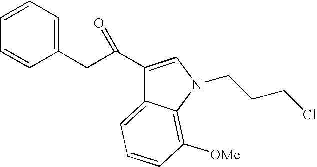

Prepared according to TP3, see also Scheme 2, by using 7-methoxy-1H-indole (286 mg, 1.94 mmol), Et₂AlCl (3.0 mL, 1.0 M, 3.0 mmol), 2-phenylacetyl chloride (0.39 mL, 3.0 mmol) in CH₂Cl₂ (10 mL) and Cs₂CO₃ (1.50 g, 4.63 mmol), 1-chloro-3-iodopropane (0.70 mL, 6.5 mmol) in CH₃CN (10 mL) to give the title compound (332 mg, 50% over two steps).

¹H NMR (400 MHz, CDCl₃) δ 8.06 (dd, J=8.1, 0.9 Hz, 1H), 7.72 (s, 1H), 7.36-7.16 (m, 6H), 6.72-6.70 (m, 1H), 4.55 (t, J=6.4 Hz, 2H), 4.11 (s, 2H), 3.92 (s, 3H), 3.41-3.38 (m, 2H), 2.31-2.24 (m, 2H).

¹³C NMR (100 MHz, CDCl₃) δ 192.5, 146.9, 136.2, 135.8, 129.2, 129.2, 128.4, 126.4, 125.8, 123.3, 115.9, 115.2, 104.2, 55.2, 47.0, 46.9, 41.5, 33.6.

### 1-(3-chloropropyl)-7-methoxy-1H-indole

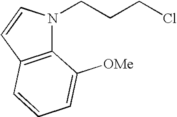

A dry round bottomed flask was charged with KOH (574 mg, 10.2 mmol) which was finely ground under Ar. To this powder was added DMSO (15 mL) and 7-methoxy-1H-indole (0.90 mL, 6.89 mmol). The suspension was submitted to ultrasound irradiation for 30 min and cooled to 0° C. To this mixture was added 1-bromo-3-chloropropane (2.00 mL, 20.3 mmol) and the mixture was left with stirring overnight. The solution was poured into ice-water (25 mL) and extracted with EtOAc (4×50 mL). The combined organic layers were washed with H₂O, brine, dried over Na₂SO₄ and adsorbed onto celite. Purification was by flash chromatography (heptanes→heptanes:EtOAc 4:1) to give the title compound (1.40 g, 91%).

¹H NMR (400 MHz, CDCl₃) δ 7.20 (dd, J=8.0, 0.9 Hz, 1H), 7.03-6.97 (m, 2H), 6.63-6.61 (m, 1H), 6.43 (d, J=3.1 Hz, 1H), 4.54 (t, J=6.4 Hz, 2H), 3.93 (s, 3H), 3.46-3.43 (m, 2H), 2.30-2.24 (m, 2H).

¹³C NMR (100 MHz, CDCl₃) δ 147.4, 131.2, 129.3, 119.9, 113.8, 113.8, 102.2, 101.3, 55.2, 46.1, 42.1, 34.6.

### 1-(3-chloropropyl)-7-ethyl-1H-indole

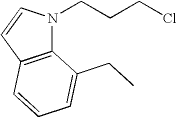

A dry round bottomed flask was charged with KOH (1.10 g, 19.7 mmol) which was finely ground under Ar. To this powder was added DMSO (30 mL) and 7-ethyl-1H-indole (2.00 mL, 14.6 mmol). The suspension was submitted to ultrasound irradiation for 30 min and cooled to 0° C. To this mixture was added 1-bromo-3-chloropropane (4.30 mL, 43.7 mmol) and the mixture was left with stirring overnight. The solution was poured into ice-water (25 mL) and extracted with EtOAc (4×50 mL). The combined organic layers were washed with H₂O, brine, dried over Na₂SO₄ and adsorbed onto celite. Purification was by flash chromatography (heptanes→heptanes:EtOAc 10:1) to give the title compound (875 mg, 20%).

¹H NMR (400 MHz, CDCl₃) δ 7.48 (dd, J=7.6, 1.5 Hz, 1H), 7.08-6.99 (m, 3H), 6.50 (d, J=6.5 Hz, 1H), 4.48 (t, J=6.7 Hz, 2H), 3.48-3.45 (m, 2H), 3.07-3.01 (m, 2H), 2.25-2.17 (m, 2H), 1.36 (t, J=7.5 Hz, 3H).

¹³C NMR (100 MHz, CDCl₃) δ 130.3, 129.8, 127.2, 122.7, 119.9, 119.1, 102.1, 45.5, 41.8, 34.5, 25.6, 15.9. (signal for C_{7b} was not observed).

### 1-(3-chloropropyl)-N-(3,4-dichlorobenzyl)-7-ethyl-1H-indole-3-carboxamide

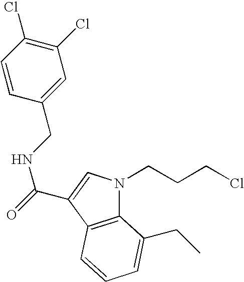

Prepared according to TP5, see also Scheme 4, by using MgI₂ (559 mg, 2.01 mmol), 1-(3-chloropropyl)-7-ethyl-1H-indole (430 mg, 1.94 mmol), 1,2-dichloro-4-(isocyanatomethyl)benzene (0.30 mL, 2.0 mmol) in DCE (4 mL) to give the title compound as off-white crystals (564 mg, 69%).

¹H NMR (400 MHz, dmso-d₆) δ 8.05-8.47 (m, 1H), 8.51-8.03 (m, 1H), 7.99 (s, 1H), 7.57-7.57-7.54 (m, 2H), 7.33-7.30 (m, 1H), 7.07-6.98 (m, 2H), 4.46-4.42 (m, 4H), 3.68-3.65 (m, 2H), 3.03-2.99 (m, 2H), 2.23-2.18 (m, 2H), 1.27-1.23 (m, 3H).

¹³C NMR (100 MHz, dmso-d₆) δ 165.0, 142.4, 134.3, 133.1, 131.5, 131.1, 129.9, 129.8, 128.7, 128.3, 128.2, 123.9, 121.8, 120.0, 110.4, 46.7, 43.1, 41.7, 34.8, 25.4, 16.7.

### N-benzyl-1-(3-chloropropyl)-7-ethyl-1H-indole-3-carboxamide

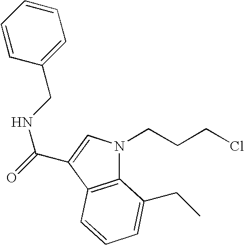

Prepared according to TP5, see also Scheme 4, by using MgI₂ (570 mg, 2.05 mmol), 1-(3-chloropropyl)-7-ethyl-1H-indole (430 mg, 1.94 mmol), (isocyanatomethyl)benzene (0.27 mL, 2.2 mmol) in DCE (4 mL) to give the title compound as off-white crystals (445 mg, 65%).

¹H NMR (400 MHz, dmso-d₆) δ 8.42-8.39 (m, 1H), 8.08-8.06 (m, 1H), 8.00 (s, 1H), 7.33-7.28 (m, 4H), 7.23-7.19 (m, 1H), 7.06-6.97 (m, 2H), 4.46-4.42 (m, 4H), 3.68-3.65 (m, 2H), 3.03-2.99 (m, 2H), 2.23-2.18 (m, 2H), 1.27-1.23 (m, 3H).

¹³C NMR (100 MHz, dmso-d₆) δ 164.9, 141.0, 134.3, 133.0, 128.9, 128.8, 27.3, 123.9, 121.6, 120.1, 110.7, 46.7, 43.1, 42.6, 34.8, 25.4, 16.7.

### 1-(3-chloropropyl)-7-methoxy-N-(3-methylbenzyl)-1H-indole-3-carboxamide

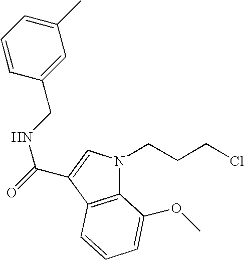

Prepared according to TP5, see also Scheme 4, by using MgI₂ (681 mg, 2.45 mmol), 1-(3-chloropropyl)-7-methoxy-1H-indole (538 mg, 2.40 mmol), 1-(isocyanatomethyl)-3-methylbenzene (0.34 mL, 2.45 mmol) in DCE (5 mL) to give the title compound as colorless crystals after flash chromatography (heptanes→heptanes:EtOAc 7:3) followed by recrystallization from EtOAc/heptanes (438 mg, 49%).

¹H NMR (400 MHz, dmso-d₆) δ 8.35-8.32 (m, 1H), 7.93 (s, 1H), 7.76-7.73 (m, 1H), 7.20-7.17 (m, 1H), 7.12-7.10 (m, 2H), 7.04-7.00 (m, 2H), 6.72 (d, J=7.6 Hz, 1H), 4.49-4.40 (m, 4H), 3.88 (s, 3H), 3.60-3.57 (m, 2H), 2.27 (s, 3H), 2.23-2.18 (m, 2H).

### 1-(3-chloropropyl)-N-(3-fluorobenzyl)-7-methoxy-1H-indole-3-carboxamide

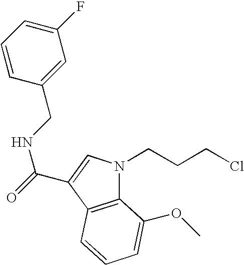

Prepared according to TP5, see also Scheme 4, by using MgI₂ (745 mg, 1-(3-chloropropyl)-7-methoxy-1H-indole (600 mg, 2.68 mmol), 1-fluoro-3-(isocyanomethyl)benzene (0.34 mL, 2.68 mmol) in DCE (5 mL) to give the title compound as colorless crystals after flash chromatography (heptanes→heptanes:EtOAc 7:3) followed by recrystallization from EtOAc/heptanes (531 mg, 53%) as a 1:1 mixture of the chloride and the iodide.

¹H NMR (400 MHz, dmso-d₆) δ 8.44-8.41 (m, 1H), 7.94 (s, 1H), 7.75-7.72 (m, 1H), 7.37-7.32 (m, 1H), 7.17-7.10 (m, 2H), 7.06-7.00 (m, 2H), 6.73 (d, J=7.6 Hz, 1H), 4.50-4.40 (m, 4H), 3.89 (s, 3H), 3.59 (t, J=6.2 Hz, 2H), 2.26-2.19 (m, 2H).

### 1-(3-chloropropyl)-7-methoxy-N-(2-methylbenzyl)-1H-indole-3-carboxamide

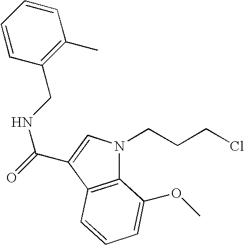

Prepared according to TP5, see also Scheme 4, by using MgI₂ (745 mg, 2.68 mmol), 1-(3-chloropropyl)-7-methoxy-1H-indole (600 mg, 2.68 mmol), 1-(isocyanatomethyl)-2-methylbenzene (0.37 mL, 2.68 mmol) in DCE (5 mL) to give the title compound as colorless crystals after flash chromatography (heptanes→heptanes:EtOAc 7:3) followed by recrystallization from EtOAc l heptanes (390 mg, 39%).

¹H NMR (400 MHz, dmso-d₆) δ 8.23-8.20 (m, 1H), 7.96 (s, 1H), 7.76-7.73 (m, 1H), 7.28-7.26 (m, 1H), 7.14-7.11 (3H), 7.03-6.99 (m, 1H), 6.72 (d, J=7.6 Hz, 1H), 4.49-4.39 (m, 4H), 3.88 (s, 3H), 3.60-3.57 (m, 2H), 2.31 (s, 3H), 2.26-2.20 (m, 2H).

### 7-Cyano-1H-indole

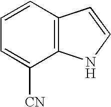

Pd(PPh₃)₄ (103 mg, 0.09 mmol) was added to 7-bromo-1H-indole (588 mg, 3 mmol) and Zn(CN)₂ in degassed DMF (10 ml). The vial was capped and heated in the MW at 170° C. for 15 min. The reaction mixture was diluted with EtOAc and washed with water and brine, dried over sodium sulphate, filtered and concentrated in vacuo. The product was purified by column chromatography (heptane 100% to heptane/EtOAc 9:1) followed by recrystallization from heptane. Yield: 350 mg (82%).

¹H NMR (400 MHz, CDCl₃) δ 8.74 (br s, 1H), 7.86 (d, 1H, J=8.0 Hz), 7.52 (d, 1H, J=7.6 Hz), 7.34 (t, 1H, J=2.6 Hz), 7.17 (t, 1H, J=8.0 Hz), 6.65 (m, 1H).

### 3-acetyl-1-(3-chloropropyl)-7-cyano-indole

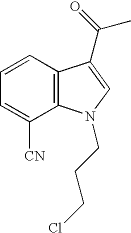

Prepared accordingly to TP3, see also Scheme 2, using 7-cyano-11H-indole (350 mg, 2.45 mmol), Et₂AlCl (3.7 mL, 1.0 M, 3.7 mmol), acetyl chloride (0.22 mL, 3.7 mmol) in CH₂Cl₂ (10 mL) and Cs₂CO₃ (0.88 g, 2.7 mmol), NaI (10 mg), 1-chloro-3-iodopropane (0.43 mL, 4.1 mmol) in DMF (10 mL), 45 min at 100° C. to give the title compound (280 mg, 43% over two steps).

¹H NMR (400 MHz, CDCl₃) δ 8.70 (m, 1H), 7.84 (s, 1H), 7.62 (m, 1H), 7.33 (t, 1H, J=8 Hz), 4.73 (t, 2H, J=6.8 Hz), 3.55 (t, 2H, J=5.6 Hz), 2.54 (s, 3H), 2.42 (m, 2H).

### 1-(1-(3-Chloropropyl)-7-methoxy-1H-indol-3-yl)ethanone

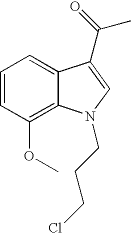

Prepared according to TP1, see also Scheme 1, using 1-(7-methoxy-1H-indol-3-yl)ethanone (500 mg, 2.65 mmol), Cs₂CO₃ (1.73 g, 5.3 mmol), NaI (cat) and 1-chloro-3-iodopropane (827 μmol, 7.94) in dry CH₃CN (10 ml). Purification by flash chromatography (heptane/ethyl acetate 99:1-1:1) gave 675 mg (97%) of title compound as white crystals.

¹H NMR (400 MHz, CDCl₃) δ 7.97 (d, 1H, J=8.0 Hz), 7.67 (s, 1H), 7.18 (t, 1H, J=8 Hz), 6.72 (d, 1H J=7.8 Hz), 4.58 (t, 2H, J=6.4 Hz), 3.94 (s, 3H), 3.47 (t, 2H, J=6.0 Hz), 2.51 (s, 3H), 2.32 (m, 2H).

### 1-(3-chloropropyl)-N-methoxy-N-methyl-1H-indole-3-carboxamide

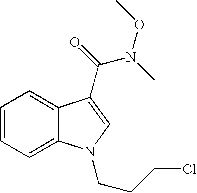

Prepared according to TP3, see also Scheme 2, from N-methoxy-N-methyl-1H-indole-3-carboxamide (865 mg, 4.24 mmol). The product was purified by flash chromatography 0-40% EtOAc in heptane to yield the title compound: 886 mg (75%).

¹H NMR (400 MHz, CDCl₃) δ 8.41 (d, J=8.0 Hz, 1H), 7.96 (s, 1H), 7.41-7.25 (m, 3H), 4.41 (t, J=6.7 Hz, 2H), 3.77 (s, 3H), 3.45 (d, J=6.7 Hz, 2H), 3.40 (s, 3H), 2.31 (pent, J=6.7 Hz, 2H).

Amines

### 1-(3-phenoxypropyl)-1,4-diazepane

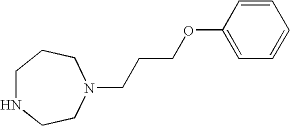

Prepared according to TP9, see also Scheme 7, by using tert-butyl 1,4-diazepane-1-carboxylate (390 mg, 1.95 mmol), (3-bromopropoxy)benzene (598 mg, 2.78 mmol), Cs₂CO₃ (1.03 g, 3.17 mmol) to give the title compound (343 mg, 75%).

¹H NMR (400 MHz, CDCl₃) δ 7.29-7.25 (m, 2H), 6.94-6.89 (m, 3H), 4.03-4.00 (m, 2H), 3.01-2.95 (m, 4H), 2.75-2.68 (m, 6H), 1.96-1.91 (m, 2H), 1.85-1.79 (m, 2H).

¹³C NMR (100 MHz, CDCl₃) δ 159.3, 129.6, 120.8, 114.8, 66.3, 57.4, 55.1, 54.6, 48.8, 47.1, 29.9, 27.8.

### 3-(2-(4-fluorophenoxy)ethyl)-3,8-diazabicyclo[3.2.1]octane

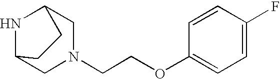

Prepared according to TP9, see also Scheme 7, by using tert-butyl 3,8-diazabicyclo[3.2.1]octane-8-carboxylate (248 mg, 1.17 mmol), 1-(2-bromoethoxy)-4-fluorobenzene (384 mg, 1.75 mmol), Cs₂CO₃ (571 mg, 1.75 mmol) in CH₃CN (10 mL) to give the title compound (188 mg, 64%).

¹H NMR (400 MHz, CDCl₃) δ 6.89-6.84 (m, 2H), 6.75-6.72 (m, 2H), 3.92-3.89 (m, 2H), 3.33-3.30 (m, 2H), 2.64-2.60 (m, 4H), 2.55 (br s, 1H), 2.25-2.23 (m, 2H), 1.77-1.75 (m, 2H), 1.63-1.59 (m, 2H).

### 4-(3-chlorophenoxy)piperidine

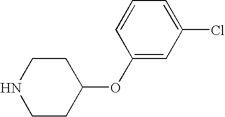

Prepared according to TP6, see also Scheme 5, from tert-butyl 4-hydroxypiperidine-1-carboxylate (400 mg, 2 mmol) and 3-chlorophenol (358 mg, 2.8 mmol). The crude product was purified by flash chromatography 0-20% EtOAc in heptane. The resulting product was dissolved in CH₂Cl₂ (10 mL) and TFA (5 mL) was cautiously added. After stirring at room temperature for 3 h the mixture was evaporated to dryness. The crude product was dissolved in a minimal amount of MeOH and purified by solid phase extraction using a SCX cartridge eluding with NH₃(MeOH) to give the title compound 304 mg (72% over two steps).

¹H NMR (400 MHz, CDCl₃) δ: 7.11-7.06 (m, 1H), 6.97-6.85 (m, 2H), 6.71-6.69 (m, 1H), 4.44-4.40 (m, 1H), 3.71-3.63 (m, 2H), 3.42-3.37 (m, 2H), 1.95-1.88 (m, 2H), 1.79-1.72 (m, 2H).

### 4-(2-phenoxyethyl)piperidine

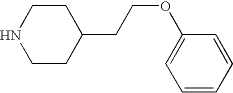

Prepared according to TP6, see also Scheme 5, using tert-butyl 4-(2-hydroxyethyl)piperidine-1-carboxylate (458 mg, 2 mmol) and phenol (263 mg, 2.8 mmol). The crude product was purified by flash chromatography 0-20% EtOAc in heptane. The resulting product was dissolved in CH₂Cl₂ (10 mL) and TFA (5 mL) was cautiously added. After stirring at room temperature for 3 h the mixture was evaporated to dryness. The crude product was dissolved in a minimal amount of MeOH and purified by solid phase extraction using a SCX cartridge eluding with NH₃(MeOH) to give the title compound 293 mg (56% over two steps).

¹H NMR (400 MHz, CDCl₃) δ: 7.31-7.25 (m, 2H), 6.98-6.88 (m, 2H), 4.13-4.06 (m, 2H), 4.00 (t, J=7.2 Hz, 2H), 2.78-2.69 (m, 2H), 1.78-1.65 (m, 5H), 1.23-0.19 (m, 2H).

¹³C NMR (100 MHz, CDCl₃) δ: 159.2, 129.6, 120.8, 114.7, 65.5, 46.6, 36.5, 33.3, 31.7.

### 4-(2-(4-chlorophenoxy)ethyl)piperidine

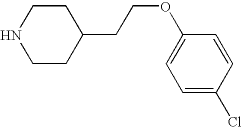

Prepared according to TP6, see also Scheme 5, from tert-butyl 4-(2-hydroxyethyl)piperidine-1-carboxylate (458 mg, 2 mmol) and 4-chlorophenol (361 mg, 2.8 mmol). The crude product was purified by flash chromatography 0-20% EtOAc in heptane. The resulting product was dissolved in CH₂Cl₂ (10 mL) and TFA (5 mL) was cautiously added. After stirring at room temperature for 3 h the mixture was evaporated to dryness. The crude product was dissolved in a minimal amount of MeOH and purified by solid phase extraction using a SCX cartridge eluding with NH₃(MeOH) to give the title compound 406 mg (86% over two steps).

¹H NMR (400 MHz, CDCl₃) δ: 7.20-7.18 (m, 2H), 6.81-6.78 (m, 2H), 4.16-4.07 (m, 2H), 3.97 (t, J=7.2 Hz, 2H), 2.74-2.67 (m, 2H), 1.77-1.60 (m, 5H), 1.20-1.11 (m, 2H).

### tert-butyl 3β-hydroxy-8-azabicyclo[3.2.1]octane-8-carboxylate

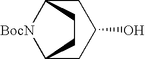

A reaction flask was charged with 3α-hydroxy-8-azabicyclo[3.2.1]octane-8-carboxylic acid tertbutyl ester (3.94 g, 17.3 mmol), 4-nitrobenzoic acid (11.3 g, 67.8 mmol), PPh₃ (18.8 g, 68.6 mmol) in dry THF (140 mL) and cooled to 0° C. Diisopropylazodicarboxylate (13.8 mL, 70.1 mmol) was added drop wise over 15 min. After 1 h cooling was removed and the mixture stirred at rt overnight. The mixture was then stirred at 45° C. for 5 h followed by cooling to rt and the mixture was diluted with diethylether and washed with several portions of saturated aqueous NaHCO₃. The combined aqueous layers were extracted with diethyl ether and the combined organic layers dried over Na₂SO₄ and evaporated to dryness. The resulting gum was stirred with diethyl ether (80 mL) while n-heptane (40 mL) was added slowly to cause crystallization. The mixture was filtered and the filter cake extracted with diethyl ether:n-heptane 1:1 (300 mL). The filtrate was adsorbed onto celite and purified by flash column chromatography (SiO₂; n-heptane→n-heptane/ethyl acetate 7:3) to give the intermediate benzoic acid ester. The ester was dissolved in THF (40 mL) and LiOH.H₂O (0.60 g, 14.3 mmol) in water (7 mL) was added. After stirring at rt for 5 h saturated aqueous NaHCO₃ was added and the mixture extracted with diethyl ether. The combined organic layers were washed with saturated aqueous NaHCO₃, brine, dried over Na₂SO₄ and evaporated to dryness to give the title compound as colorless crystals (3.33 g, 82%).

### (1R,3r,5S)-3-(4-fluorophenoxy)-8-azabicyclo[3.2.1]octane

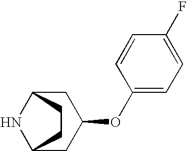

Prepared according to TP7, see also Scheme 5, from 4-fluorophenol (313 mg, 2.8 mmol) and tert-butyl 3β-hydroxy-8-azabicyclo[3.2.1]octane-8-carboxylate (451 mg, 2.0 mmol). Yield: 130 mg (29% over two steps).

¹H NMR (400 MHz, CDCl₃) δ: 6.95-6.90 (m, 2H), 6.78-6.72 (m, 2H), 4.53-4.49 (m, 1H), 4.23-4.11 (m, 2H), 2.17-1.90 (m, 8H).

### (1R,3r,5S)-3-(2-chlorophenoxy)-8-azabicyclo[3.2.1]octane

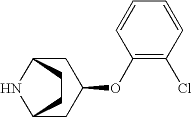

Prepared according to TP7, see also Scheme 5, from 2-chlorophenol (358 mg, 2.8 mmol) and tert-butyl 3β-hydroxy-8-azabicyclo[3.2.1]octane-8-carboxylate (451 mg, 2.0 mmol). Yield: 263 mg (58% over two steps).

¹H NMR (400 MHz, CDCl₃) δ: 7.34 (d, J=6.6 Hz, 1H), 7.18 (t, J=6.6 Hz, 1H), 6.83 (t, J=6.6 Hz, 1H), 6.79 (d, J=6.6 Hz, 1H), 4.64-4.60 (m, 1H), 4.23-4.18 (m, 2H), 2.32-1.95 (m, 8H).

### 3-Ethoxycarbonylmethylene-8-azabicyclo[3.2.1]octane-8-carboxylic acid t-butyl ester

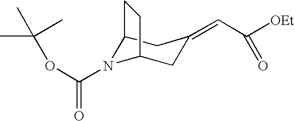

A reaction flask was charged with triethyl phosphonoacetate (7.458 g, 33.3 mmol) in dry THF (20 mL) under Argon. NaH (60% in mineral oil, 1.33 g, 33.3 mmol) was added in portions and the mixture was stirred at rt for 1 h. The clear solution was cooled to <10° C. with an icebath followed by dropwise addition of 3-oxo-8-azabicyclo[3.2.1]octane-8-carboxylic acid t-butyl ester (4.977 g, 22.2 mmol) dissolved in THF (5 mL) over 45 min. The temperature was slowly raised to rt and the reaction was stirred for another 20 h. The reaction mixture was quenched with water and the product extracted into ethyl acetate. The combined organic phases were dried over sodium sulfate, filtered, and concentrated. The product was purified by flash column chromatography (SiO₂; n-heptane/ethyl acetate 4:1) to give the title compound: 5.416 g (82%).

¹H NMR (CDCl₃) δ 5.76-5.74 (m, 1H), 4.28 (br s, 2H), 4.19-4.07 (m 2), 3.66-3.59 (m, 1H), 2.76-2.20 (m, 2H), 2.11-2.06 (m, 1H), 1.93-1.87 (m, 2H), 1.58-1.54 (m, 2H) δ 1.46 (m, 9H), 1.26 (t, 3H).

### (8-Azabicyclo[3.2.1]oct-3-ylidene)acetic acid ethyl ester

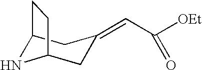

To 3-ethoxycarbonylmethylene-8-azabicyclo[3.2.1]octane-8-carboxylic acid t-butyl ester (12.3 g, 41.8 mmol) in dichloromethane was added TFA (10 mL) and the reaction was stirred for 8 h. The solution was concentrated under reduced pressure, diluted with dichloromethane, and washed with 2 M NaOH followed by brine. The water phases were thereafter back-extracted with ethyl acetate and the combined organic phases were dried over sodium sulfate, filtered, and concentrated. The crude product: 7.04 g (91%) was used without further purification.

### (8-Azabicyclo[3.2.1]oct-3α-yl)acetic acid ethyl ester

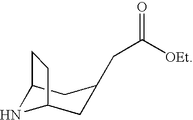

A 250 mL reaction flask was charged with (8-azabicyclo[3.2.1]oct-3-ylidene)acetic acid ethyl ester (3.7 g, 19 mmol), ammonium formiate (14 g, 190 mmol), and Pd/C (0.32 g) in 150 mL MeOR. When all of the ammonium formiate was dissolved the mixture was degassed for 15 min. The reaction was stirred on under an inert atmosphere (N2) over night at rt. The mixture was filtered through celite, concentrated, diluted with 2 M NaOH (ca pH 10), and extracted with ethyl acetate. The combined organic phases were washed with brine, dried over sodium sulfate, filtered, and concentrated to give the crude product 3.1 g (83%; 85:15 α:β) that was used without further purification. Major isomer:

¹H NMR (CDCl₃) δ 4.08 (q, J=7.2 Hz, 2H), 3.45-3.41 (m, 2H), 2.40 (d, J=8.0 Hz, 2H), 2.25-2.18 (m, 1H), 2.06-1.96 (m, 2H), 1.82-1.55 (m, 5H), 1.31-1.23 (m, 2H), 1.21 (to J=7.2 Hz, 3H).

¹³C NMR (CDCl₃) δ 173.3, 60.4, 53.6, 42.6, 36.7, 30.4, 25.4, 14.4.

### 3α-Ethoxycarbonylmethyl-8-azabicyclo[3.2.1]octane-8-carboxylic acid tert-butyl ester

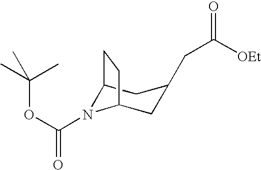

A solution of di-tert-butyldicarbonate (4.3 g, 20 mmol) in THF (10 mL) was added to a cooled (ice water bath) solution of (8-azabicyclo[3.2.1]oct-3α-yl)acetic acid ethyl ester (2.8 g, 14 mmol) in THF (40 mL). The reaction was stirred at rt for 14 h and then concentrated. The semi-solid residue was diluted with ethyl acetate, washed with brine, dried over sodium sulfate, filtered, and concentrated. The oily residue was purified by flash column chromatography (SiO₂; heptane/ethyl acetate 7:3) to yield the title compound: 3.8 g (73%) as an oil. Major isomer:

¹H NMR (CDCl₃) δ 4.16 (vbr s, 2H), 4.11 (q, J=6.8 Hz, 2H), 2.43 (d, J=7.6 Hz, 2H), 2.24-2.12 (m, 3H), 2.00-1.92 (m, 2H), 1.70-1.61 (M, 2H), 1.44 (s, 9H), 1.23 (t, J=6.8 Hz, 3H).

### [(1R,5S)-8-(tert-butoxycarbonyl)-8-azabicyclo[3.2.1]oct-3α-yl]acetic acid

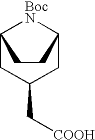

3α-Ethoxycarbonylmethyl-8-azabicyclo[3.2.1]octane-8-carboxylic acid tert-butyl ester (5.38 g, 18 mmol) was taken up in THF (30 mL) and NaOH (2M, 20 mL) and the reaction mixture was stirred at rt for 48 h. The THF was distilled off and the aq. phase made acidic with HCl (pH=2) before extraction of the product with ether. The organic phase was dried over Na₂SO₄ and concentrated in vacuo. Yield: 4.87 g (99%).

### 2-((1R,3r,5S)-8-azabicyclo[3.2.1]octan-3-yl)-1-(4-chlorophenyl)ethanone

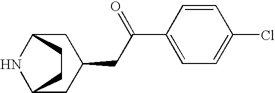

Prepared according to TP10, see also Scheme 8, from [(1R,5S)-8-(tert-butoxycarbonyl)-8-azabicyclo[3.2.1]oct-3α-yl]acetic acid (1.34 g, 5 mmol) and 4-chlorophenylmagnesium chloride (10 mL, 1 M, 10 mmol). After extractive work up the product was purified by flash chromatography 0-20% EtOAc in heptane. Yield 1.48 g (82%). The product (386 mg, 1 mmol) was taken up in a mixture of TFA (2 mL) and DCM (2 mL) and stirred for 1 h at rt, concentrated in vacuo, then taken up in EtOAc, washed with NaOH (2 N), dried over Na₂SO₄ and concentrated in vacuo. Yield: 278 mg (97%).

¹H NMR (400 MHz, CDCl₃) δ: 7.84 (d, J=6.8 Hz, 2H), 7.45 (d, J=6.8 Hz, 2H), 4.21-4.11 (m, 2H), 3.09-3.02 (m, 2H), 2.38-1.93 (m, 4H), 1.71-1.63 (m, 3H), 1.24-1.19(m, 2H).

¹³C NMR (100 MHz, CDCl₃) δ: 198.8, 139.7, 135.6, 129.7, 129.2, 56.5, 53.6, 36.9, 30.5, 24.5.

### (1R,3r,5S)-3-(4-chlorophenethyl)-8-azabicyclo[3.2.1]octane

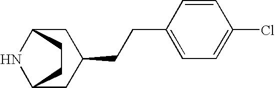

Prepared according to TP11, see also Scheme 9, from (1R,3r,5S)-tert-butyl 3-(2-(4-chlorophenyl)-2-oxoethyl)-8-azabicyclo[3.2.1]octane-8-carboxylate (928 mg, 2.6 mmol). After extractive workup, the product was purified by SCX. Yield: 310 mg (48%).

¹H NMR (400 MHz, CDCl₃) δ: 7.24-7.20 (m, 2H), 7.07-7.01 (m, 2H), 3.52-3.43 (m, 2H), 2.55-2.50 (m, 1H), 2.07-1.98 (m, 2H), 1.80-1.66 (m, 6H), 1.32-1.27 (m, 2H).

### 2-((1R,3r,5S)-8-azabicyclo[3.2.1]octan-3-yl)-1-(3,4-dichlorophenyl)ethanone

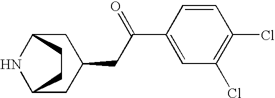

Prepared according to TP10, see also Scheme 8, from [(1R,5S)-8-(tert-butoxycarbonyl)-8-azabicyclo[3.2.1]oct-3α-yl]acetic acid (468 mg, 1.5 mmol) and 3,4-dichlorophenylmagnesium chloride (6 mL, 0.5 M, 3 mmol). After extractive work up the product was purified by flash chromatography 0-20% EtOAc in heptane. The product was taken up in a mixture of TFA (2 mL) and DCM (2 mL), stirred for 1 h at rt and purified by SCX. Yield: 300 mg (68%).

¹H NMR (400 MHz, CDCl₃) δ: 7.97 (d, J=2.0 Hz, 1H), 7.72 (dd, J=2.0 and 8.6 Hz, 1H), 7.52 (d, J=8.6 Hz, 1H), 3.56-3.54 (m, 2H), 3.04 (d, J=7.5 Hz, 2H), 2.50-2.45 (m, 1H), 2.18-2.11 (m, 2H), 1.92-1.89 (m, 2H), 1.77-1.74 (m, 2H), 1.34-1.29 (m, 2H).

¹³C NMR (100 MHz, CDCl₃) δ: 197.6, 137.8, 136.8, 133.6, 131.0, 130.3, 127.3, 53.5, 46.4, 36.7, 30.3, 24.3.

### 2-((1R,3r,5S)-8-azabicyclo[3.2.1]octan-3-yl)-1-(3,5-dichlorophenyl)ethanone

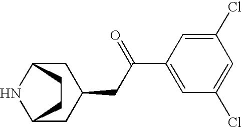

Prepared according to TP10, see also Scheme 8, from [(1R,5S)-8-(tert-butoxycarbonyl)-8-azabicyclo[3.2.1]oct-3α-yl]acetic acid (468 mg, 1.5 mmol) and 3,5-dichlorophenylmagnesium chloride (6 mL, 0.5 M, 3 mmol). After extractive work up the product was purified by flash chromatography 0-20% EtOAc in heptane. The product was taken up in a mixture of TFA (2 mL) and DCM (2 mL), stirred for 1 h at rt and purified by SCX. Yield: 260 mg (58%).

¹H NMR (400 MHz, CDCl₃) δ: 7.75 (d, J=1.5 Hz, 2H), 7.58 (t, J=1.5 Hz, 1H), 4.05-3.98 (m, 2H), 4.36 (d, J=7.8 Hz, 2H), 2.68-2.62 (m, 1H), 2.53-2.45 (m, 2H), 2.34-2.29 (m, 2H), 2.05-1.97 (m, 2H), 1.67-1.60 (m, 2H).

¹³C NMR (100 MHz, CDCl₃) δ: 195.8, 139.0, 136.2, 133.4, 126.6, 54.2, 45.8, 33.0, 26.9, 22.8.

### 2-((1R,3r,5S)-8-azabicyclo[3.2.1]octan-3-yl)-1-(3-chlorophenyl)ethanone

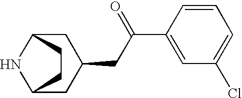

Prepared according to TP10, see also Scheme 8, from [(1R,5S)-8-(tert-butoxycarbonyl)-8-azabicyclo[3.2.1]oct-3α-yl]acetic acid (468 mg, 1.5 mmol) and 3-chlorophenylmagnesium chloride (6 mL, 0.5 M, 3 mmol). After extractive work up the product was purified by flash chromatography 0-20% EtOAc in heptane. The product was taken up in a mixture of TFA (2 mL) and DCM (2 mL), stirred for 1 h at rt and purified by SCX. Yield: 290 mg (66%).

¹H NMR (400 MHz, CDCl₃) δ: 7.91-7.86 (m, 1H), 7.82-7.76 (m, 1H), 7.57-7.52 (m, 1H), 7.44-7.38 (m, 1H), 4.02-3.98 (m, 2H), 3.22-3.18 (m, 2H), 2.27-2.65 (m, 1H), 2.54-2.46 (m, 2H), 2.33-2.21 (m, 2H), 2.02-1.97 (m, 2H), 1.85-1.80 (m, 2H).

¹³C NMR (100 MHz, CDCl₃) δ: 196.5, 138.2, 135.1, 134.7, 131.9, 128.2, 126.3, 55.4, 46.0, 32.2, 26.4, 23.4.

### 2-((1R,3r,5S)-8-azabicyclo[3.2.1]octan-3-yl)-1-(pyridin-2-yl)ethanone

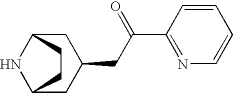

Prepared according to TP10, see also Scheme 8, from [(1R,5S)-8-(tert-butoxycarbonyl)-8-azabicyclo[3.2.1]oct-3α-yl]acetic acid (468 mg, 1.5 mmol) and 2-pyridininmagnesium chloride (12 mL, 0.25 M, 3 mmol). After extractive work up the product was purified by flash chromatography 0-20% EtOAc in heptane. The product was taken up in a mixture of TFA (2 mL) and DCM (2 mL), stirred for 1 h at rt and purified by SCX. Yield: 172 mg (50%).

¹H NMR (400 MHz, CDCl₃) δ: 8.66-8.65 (m, 1H), 8.01-7.99 (m, 1H), 7.83-7.79 (m, 1H), 7.46-7.43 (m, 1H), 3.59-3.53 (m, 2H), 3.39-3.37 (m, 2H), 2.59-2.50 (m, 1H), 2.17-2.10 (m, 2H), 1.93-1.86 (m, 4H), 1.42-1.38 (m, 2H).

¹³C NMR (100 MHz, CDCl₃) δ: 201.8, 153.8, 149.2, 137.1, 127.3, 122.1, 54.9, 45.2, 36.5, 29.8, 29.0, 28.9, 24.3.

### 2-((1R,3r,5S)-8-azabicyclo[3.2.1]octan-3-yl)-1-(3-chloro-4-fluorophenyl)ethanone

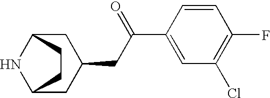

Prepared according to TP10, see also Scheme 8, from [(1R,5S)-8-(tert-butoxycarbonyl)-8-azabicyclo[3.2.1]oct-3α-yl]acetic acid (468 mg, 1.5 mmol) and 3-chloro-4-fluorophenylmagnesium chloride (6 mL, 0.5 M, 3 mmol). After extractive work up the product was purified by flash chromatography 0-20% EtOAc in heptane. The product was taken up in a mixture of TFA (2 mL) and DCM (2 mL), stirred for 1 h at rt and purified by SCX. Yield: 122 mg (21%).

¹H NMR (400 MHz, CDCl₃) δ: 8.00-7.97 (m, 1H), 7.84-7.80 (m, 1H), 7.26-7.19 (m, 1H), 3.57-3.55 (m, 2H), 3.10-3.04 (m, 2H), 2.48-2.46 (m, 1H), 2.19-2.17 (m, 2H), 1.93-1.89 (m, 2H), 1.79-1.77 (m, 2H), 1.35-1.31 (m, 2H).

¹³C NMR (100 MHz, CDCl₃) δ: 198.0, 134.3, 131.2, 128.6, 128.5, 117.2, 116.9, 53.5, 46.4, 36.7, 30.4, 24.3.

### 1-(2-(4-fluorophenoxy)ethyl)-1,4-diazepane

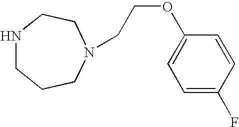

Prepared according to TP9, see also Scheme 7, from tert-butyl 1,4-diazepane-1-carboxylate (400 mg, 2 mmol), 1-(2-bromoethoxy)-4-fluorobenzene (569 mg, 2.6 mmol). Yield: 450 mg (94%).

¹H NMR (400 MHz, CDCl₃) δ: 6.96-6.91 (m, 2H), 6.82-6.79 (m, 2H), 4.38-3.34 (m, 2H), 4.00 (t, J=5.4 Hz, 2H), 3.05-3.00 (m, 2H), 2.95-2.80 (m, 4H), 1.86-1.83 (m, 2H).

¹³C NMR (100 MHz, CDCl₃) δ: 162.5, 158.7, 156.3, 155.1, 116.1, 115.9, 115.8, 115.7, 67.3, 56.7, 55.8, 54.9, 47.8, 46.3, 28.6.

### 1-(2-phenoxyethyl)-1,4-diazepane

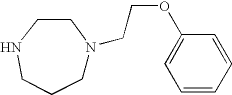

Prepared according to TP9, see also Scheme 7 from tert-butyl 1,4-diazepane-1-carboxylate (400 mg, 2 mmol), (2-bromoethoxy)benzene (569 mg, 2.6 mmol). Yield: 377 mg (86%).

¹H NMR (400 MHz, CDCl₃) δ: 7.28-7.24 (m, 2H), 6.95-6.86 (m, 3H), 5.22-5.20 (m, 2H), 4.06-4.03 (m, 2H), 3.09-2.82 (8H), 1.91-1.85 (m, 2H).

¹³C NMR (100 MHz, CDCl₃) δ: 158.9, 129.7, 121.1, 114.8, 66.6, 57.0, 55.0, 54.8, 47.8, 46.1, 28.2.

### 3-Trifluorosulfonyl-8-tertBlutloxycarbonyl-8-azabicyclo[3.2.1]-oct-2-ene (N-Boc-nortropanone enol triflate)

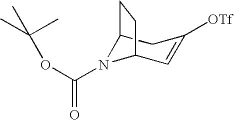

LDA was generated by adding BuLi (20 mL, 1.68M, 32.6 mmol) to a solution of diisopropylamine (2.38 g, 32.6 mmol) in dry THF (10 mL) at −78° C. under argon. The mixture was kept at that temperature for 30 min followed by the addition of a solution of N-Bocnortropinone (5.27 g, 23.4 mmol) in dry THF (20 mL). The mixture was then left stirring for 1 h while maintaining the temperature at 78° C. Then a solution of 2-[N,N-Bis(trifluoromethylsulfonyl)amino]-5-chloropyridine (10.08 g, 25.7 mmol) in dry THF (20 mL) was added and the mixture was slowly allowed to reach room temperature overnight and subsequently concentrated and purified by flash chromatography (EtOAc/heptane 1:6,) to give the title compound 6.68 g (80%) which on prolonged standing crystallised into a white solid.

¹H NMR (CDCl₃) δ 6.10 (bs, 1H), 4.42 (m, 2H), 3.05 (bs, 1H), 2.23 (m, 1H), 2.07 (d, J=16.6 Hz, 1H), 1.93-2.03 (m, 2H), 1.72 (m, 1H), 1.43 (s, 9H).

¹³C NMR (CDCl₃) δ 153.9, 148.0, 124.0, 118.7, 80.5, 51.9, 36.5, 34.7, 30.1, 28.4.

### tert-butyl (1R,5S)-3-pentyl-8-azabicyclo[3.2.1]octane-8-carboxylate

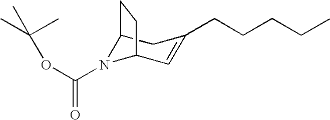

In a dry argon flushed schlenk flask palladium acetate (220 mg, 0.98 mmol) and tricyclohexyl phosphine (549 mg, 1.96 mmol) were dissolved in THF (40 mL) and NMP (20 mL). After 5 min 3-trifluorosulfonyl-8-tertButyloxycarbonyl-8-azabicyclo[3.2.1]-oct-2-ene (N-Boc-nortropanone enol triflate) (7.0 g, 19.6 mmol) and NMI (1.72 g, 21 mmol) were added. In a second dry argon flushed flask pentyl magnesium bromide (2M, 14 mL, 28 mmol) was transmetallated to the corresponding zinc reagent with ZnBr₂ (1.5 M, 18.6 mL, 28 mmol) at rt. The formed pentyl zinc reagent was added to the reaction mixture (exothermic). The reaction was heated to 80° C. for 16 h, then quenched with McOH (10 mL) and filtered through celite. The reaction mixture was diluted with EtOAc and washed with water and brine, dried over sodium sulphate, filtered and concentrated onto celite. The product was purified by flash chromatography 0-5% EtOAc in heptane. Yield: 1.92 g (73%).

¹H NMR (400 MHz, CDCl₃) δ: 5.67 (bs, 1H), 4.38-4.17 (m, 2H), 2.78-2.60 (m, 1H), 2.19-2.06 (m, 1H), 1.98-1.55 (m, 6H), 1.43 (s, 9H), 1.38-1.17 (m, 6H), 0.87 (t, J=7.2 Hz, 3H).

### (1R,5S)-3α-pentyl-8-azabicyclo[3.2.1]octane

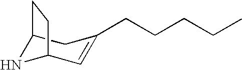

tert-butyl (1R,5S)-3-pentyl-8-azabicyclo[3.2.1]octane-8-carboxylate (450 mg, 1.7 mmol) was dissolved in DCM (2 mL) and TFA (2 mL) and left stirring for 1 h, concentrated in vacuo. The crude product was redissolved in EtOAc and washed with NaOH (2 M, aq.), dried over sodium sulphate, filtered and concentrated in vacuo. Platinum oxide (34 mg), acetic acid (100 mg, 1.7 mmol) and MeOH were added, the flask was evacuated and flushed with H₂. The reaction was stirred for 2 h at rt then filtered through celite and concentrated in vacuo. The concentrate was taken up in DCM and washed with NaOH (2 M, aq.) dried over sodium sulphate, filtered and concentrated in vacuo. Yield: 272 mg (88%).

¹H NMR (400 MHz, CDCl₃) δ: 3.41 (bs, 2H), 1.95 (pentet, J=6.9 Hz, 2H), 1.74-1.21 (m, 15H), 0.84 (t, J=7.0 Hz, 3H).

¹³C NMR (100 MHZ, CDCl₃)—major isomer: δ 53.7, 38.0, 37.4, 32.1, 30.9, 28.5, 28.3, 22.8, 14.2.

### 1-[1-[3-(4-butyl-1-piperidinyl)propyl]-1H-indol-3-yl]-ethanone C900b

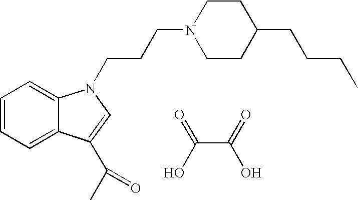

1-[1-(3-chloropropyl)-1H-indol-3-yl]-ethanone (235 mg, 1 mmol), cesium carbonate (650 mg, 2 mmol), potassium iodide (166 mg, 1 mmol) and 4-n-butylpiperidine (134 mg, 0.95 mmol) were weighed into a MW vial and dry MeCN (4 mL) was added. The vial was capped and heated in the MW at 120° C. for 20 min. The reaction was repeated 4 times. The reaction mixture was diluted with EtOAc and washed with water and brine, dried over sodium sulphate, filtered and concentrated onto celite. The product was purified by flash chromatography 0-10% MeOH in DCM. Yield: 1.07 g (78%). LC/MS purity: UV/MS 100/100.

¹H NMR (400 MHz, CDCl₃) δ: 8.38-8.36 (m, 1H), 7.82 (s, 1H), 7.40-7.26 (m, 3H), 4.26 (t, J=6.4 Hz, 2H), 2.88 (bd, 2H), 2.52 (s, 3H), 2.29 (t, J=6.8 Hz, 2H), 2.08 (pentet, J=6.8 Hz, 2H), 1.95 (bt, 2H), 1.71 (bd, 2H), 1.30-1.24 (m, 9H), 0.89 (t, J=6.8 Hz, 3H).

¹³C NMR (100 MHz, CDCl₃); δ 193.1, 137.0, 135.6, 126.6, 123.4, 122.9, 122.7, 117.2, 110.0, 54.9, 54.1, 44.7, 36.4, 35.9, 32.4, 29.2, 27.8, 26.8, 23.1, 14.3.

### 1-(1-{3-[(1R,5S)-3α-(2-phenylethyl)-8-azabicyclo[3.2.1]oct-8-yl]propyl}-1H-indol-3-yl)ethanone C616b

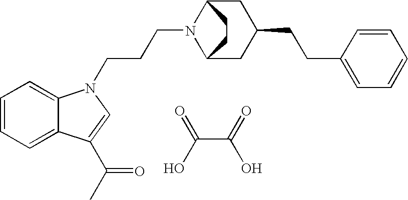

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and (1R,58)-3-(2-phenylethyl)-8-azabicyclo[3.2.1]octane (80 mg, 0.35 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 88 mg (57%). LC/MS purity: UV/MS 99/98.

¹H NMR (400 MHz, CDCl₃) δ: 8.39-8.37 (m, 1H), 7.85 (s, 1H), 7.42-7.16 (m, 8H), 4.33 (t, J=6.6 Hz, 2H), 3.20 (bs, 2H), 2.62 (t, J=7.2 Hz, 2H), 2.52 (s, 3H), 2.32 (t, J=6.6 Hz, 2H), 2.25-2.17 (m, 2H), 2.03 (pentet, J=6.6 Hz, 2H), 1.94-1.90 (m, 2H), 1.78 (t, J=5.8 Hz, 2H), 1.68-1.38 (m, 5H).

### 1-(1-{3-[(1R,5S)-3α-pentyl-8-azabicyclo[3.2.1]oct-8-yl]propyl}-1H-indol-3-yl)ethanone

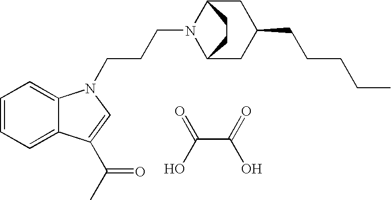

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and (1R,5S)-3-pentyl-8-azabicyclo[3.2.1]octane (81 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 123 mg (72%). LC/MS purity: UV/MS 100/92.

¹H NMR (400 MHz, CDCl₃) δ: 8.39-8.36 (m, 1H), 7.81 (s, 1H), 7.43-7.26 (m, 3H), 4.33 (t, J=6.6 Hz, 2H), 3.12-3.09 (m, 2H), 2.52 (s, 3H), 2.24 (t, J=6.6 Hz, 2H), 2.15-2.09 (m, 2H), 1.96 (pentet, J=6.6 Hz, 2H), 1.90-1.87 (m, 2H), 1.74-1.22 (m, 13H), 0.89 (t, J=7.0 Hz, 3H).

¹³C NMR (100 MHz, CDCl₃); δ 193.1, 137.1, 135.8, 126.6, 123.3, 122.8, 122.6, 117.0, 110.2, 58.8, 48.2, 44.6, 38.6, 36.1, 32.2, 28.6, 28.6, 28.5, 27.7, 27.4, 22.9, 14.3.

### N-(3-methylbenzyl)-1-{3-[(1R,5S)-3α-(2-phenylethyl)-8-azabicyclo[3.2.1]oct-8-yl]propyl}-1H-indole-3-carboxamide C296b

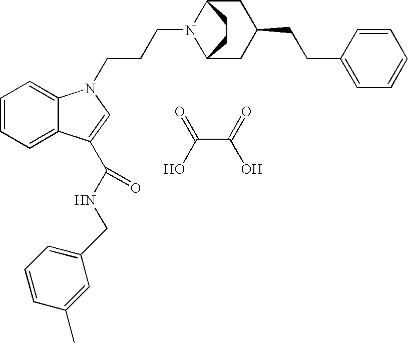

1-(3-chloropropyl)-N-(3-methylbenzyl)-1H-indole-3-carboxamide (117 mg, 0.5 mmol), cesium carbonate (325 mg, 1 mmol), potassium iodide (83 mg, 0.5 mmol) and (1R,5S)-3-pentyl-8-azabicyclo[3.2.1]octane (80 mg, 0.35 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 74 mg (41%). LC/MS purity: UV/MS 100/92.

¹H NMR (400 MHz, CDCl₃) δ: 7.98-7.81 (m, 2H), 7.44-7.42 (m, 1H), 7.29-7.10 (m, 11H), 6.15 (bt, 1H), 4.66 (d, J=5.6 Hz, 2H), 4.28 (t, J=6.6 Hz, 2H), 3.22 (bs, 2H), 2.89 (t, J=7.2 Hz, 2H), 2.62-2.58 (m, 2H), 2.37-1.36 (m, 14H), 1.27 (t, J=7.2 Hz, 2H).

### 1-(1-{3-[4-(2-methoxyphenyl)-1-piperidinyl]propyl}-1H-indol-3-yl)ethanone C639b

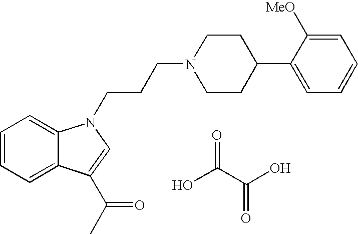

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and 4-(2-methoxyphenyl)piperidine (86 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 125 mg (71%). LC/MS purity: UV/MS 99/86.

¹H NMR (400 MHz, CDCl₃) δ: 8.28-8.26 (m, 2H), 7.59 (dt, J=8.0 and 1.0 Hz, 1H), 7.34-7.13 (m, 4H), 6.94-6.88 (m, 2H), 4.40 (t, J=6.8 Hz, 2H), 3.81 (s, 3H), 3.43-3.40 (m, 2H), 3.18-3.10 (m, 1H), 2.97-2.93 (m, 2H), 2.78-2.75 (m, 2H), 2.54 (s, 3H), 2.34-2.30 (m, 2H), 1.95-1.89 (m, 4H).

### 1-(1-{3-[4-(1,3-benzothiazol-2-yl)-1-piperidinyl]propyl}-1H-indol-3-yl)ethanone C667b

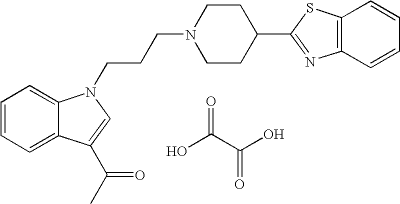

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and 2-(4-piperidinyl)-1,3-benzothiazole (98 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 100 mg (53%). LC/MS purity: UV/MS 98/84.

¹H NMR (400 MHz, CDCl₃) δ: 8.26-8.24 (m, 2H), 7.96-7.90 (m, 2H), 7.58-7.22 (m, 5H), 4.39 (t, J=6.8 Hz, 2H), 3.34-3.24 (m, 5H), 2.76-2.72 (m, 2H), 2.61-2.57 (m, 2H), 2.53 (s, 3H), 2.28-2.22 (m, 4H).

### 1-(1-{3-[4-(4-fluorophenoxy)-1-piperidinyl]propyl}-1H-indol-3-yl)ethanone C656b

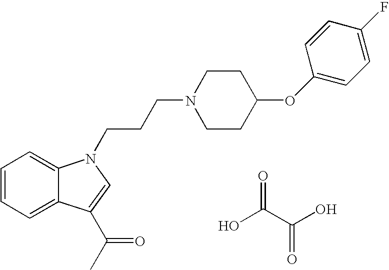

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and 4-(4-fluorophenoxy)piperidine (104 mg, 0.45 mmol) were weighed into a MW vial and dry MeCN (2 mL) was added. The vial was capped and heated in the MW at 120 CC for 20 min. The reaction mixture was diluted with EtOAc and washed with water and brine, dried over sodium sulphate, filtered and concentrated onto celite. The product was purified by flash chromatography 0-5% MeOH in DCM. Yield: 111 mg (63%). LC/MS purity: UV/MS 98/82.

¹H NMR (400 MHz, CDCl₃) δ: 8.25-8.22 (m, 2H), 7.55-7.53 (m, 1H), 7.35-7.21 (m, 2H), 7.00-6.91 (m, 4H), 4.40-4.34 (m, 3H), 2.99-2.95 (m, 2H), 2.70-2.66 (m, 4H), 2.52 (s, 3H), 2.22-2.18 (m, 2H), 2.04-2.00 (m, 2H), 1.88-1.82 (m, 2H).

### N-(3-chlorobenzyl)-1-{3-[4-(4-fluorophenoxy)-1-piperidinyl]propyl}-1H-indole-3-carboxamide C292b

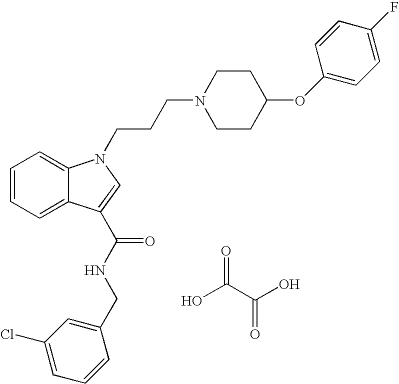

1-(3-chloropropyl)-N-(3-chlorobenzyl)-1H-indole-3-carboxamide (180 mg, 0.5 mmol), cesium carbonate (325 mg, 1 mmol), potassium iodide (83 mg, 0.5 mmol) and 4-(4-fluorophenoxy)piperidine (104 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 80 mg (34%). LC/MS purity: UV/MS 99/84.

¹H NMR (400 MHz, CDCl₃) δ: 8.14 (dt, J=8.0 and 0.6 Hz, 1H), 7.88 (s, 1H), 7.43 (dt, J=8.4 and 1.0 Hz, 1H), 7.37-7.35 (m, 1H), 7.26-7.16 (m, 5H), 6.96-6.92 (m, 2H), 6.85-6.82 (m, 2H), 4.53 (s, 2H), 4.21-4.18 (m, 3H), 2.63-2.59 (m, 2H), 2.25 (t, J=7.4 Hz, 2H), 2.21-2.13 (m, 2H), 1.98 (pentet, J=7.4 Hz, 2H), 1.91-1.86 (m, 2H), 1.71-1.63 (m, 2H).

### 1-(1-{3-[4-(2-chlorophenoxy)-1-piperidinyl]propyl}-1H-indol-3-yl)ethanone C627b

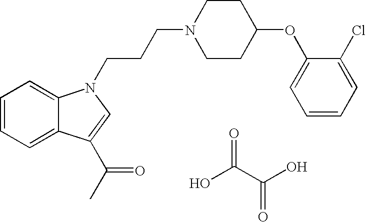

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and 4-(2-chlorophenoxy)piperidine (111 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 181 mg (98%). LC/MS purity: UV/MS 99/95.

¹H NMR (400 MHz, CDCl₃) δ: 8.28 (s, 1H), 8.24-8.22 (m, 1H), 7.59-7.57 (m, 1H), 7.35 (dd, J=8.0 and 1.6 Hz, 1H), 7.31-7.21 (m, 3H), 7.12 (dd, J=8.0 and 1.6 Hz, 1H), 6.95 (dt, J=7.2 and 1.2 Hz, 1H), 4.73.4.69 (m, 1H), 4.39 (t, J=7.2 Hz, 2H), 3.36-3.17 (m, 6H), 2.51 (s, 3H), 2.40-2.36 (m, 2H), 2.20-2.05 (m, 4H).

### 1-{1-[3-(6-methoxy-1,3,4,9-tetrahydro-2H-β-carbolin-2-yl)propyl]-1H-indol-3-yl}ethanone C901b

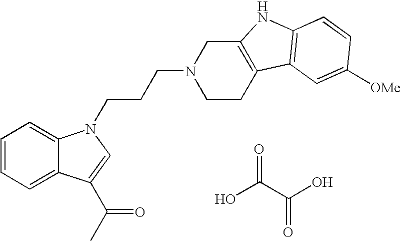

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and 6-methoxy-1,2,3,4-tetrahydro-β-carboline (91 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 66 mg (37%). LC/MS purity: UV/MS 97/58.

¹H NMR (400 MHz, CDCl₃) δ: 8.25-8.23 (m, 1H), 8.10 (s, 1H), 7.47 (d, J=8.4 Hz, 1H), 7.28-7.19 (m, 2H), 7.14 (d, J=8.8 Hz, 1H), 6.87 (d, J=2.4 Hz, 1H), 6.70 (dd, J=8.4 and 2.4 Hz, 1H), 4.26 (t, J=6.8 Hz, 2H), 3.76 (s, 3H), 3.69 (s, 2H), 2.88 (t, J=6.0 Hz, 2H), 2.76 (t, J=6.0 Hz, 2H), 2.64 (t, J=7.9 Hz, 2H), 2.43 (s, 3H), 2.15 (pentet, J=7.9 Hz, 2H).

### 1-(1-{3α-[3-(4-chlorophenoxy)-8-azabicyclo[3.2.1]oct-8-yl]propyl}-1H-indol-3-yl)ethanone C390b

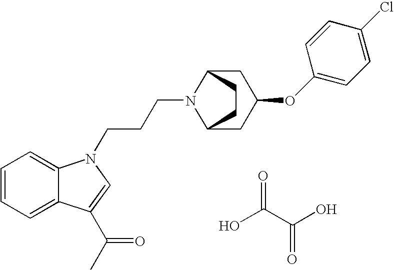

1-[1-(3-chloropropyl)-1H-indol-3-yl]-ethanone (117 mg, 0.5 mmol), cesium carbonate (325 mg, 1 mmol), potassium iodide (83 mg, 0.5 mmol) and 3α-(4-chlorophenoxy)-8-azabicyclo[3.2.1]octane (106 mg, 0.45 mmol) were weighed into a MW vial and dry MeCN (2 mL) was added. The vial was capped and heated in the MW at 120° C. for 20 min. The reaction mixture was diluted with EtOAc and washed with water and brine, dried over sodium sulphate, filtered and concentrated onto celite. The product was purified by flash chromatography 0-5% MeOH in DCM. Yield: 90 mg (46%). LC/MS purity: UV/MS 99/96.

¹H NMR (400 MHz, CDCl₃) δ: 8.39-8.36 (m, 1H), 7.81 (s, 1H), 7.42-7.40 (m, 1H), 7.30-7.21 (m, 4H), 6.76-6.73 (m, 2H), 4.50 (t, J=5.6 Hz, 2H), 4.32 (t, J=6.6 Hz, 2H), 3.13-3.12 (m, 2H), 2.52 (s, 3H), 2.29 (t, J=6.6 Hz, 2H), 2.13-1.85 (m, 10H).

¹³C NMR (100 MHz, CDCl₃) δ: 193.1, 156.2, 137.1, 135.5, 129.7, 126.6, 125.5, 123.3, 122.8, 122.7, 117.2, 116.9, 110.1, 70.5, 58.4, 48.7, 44.6, 35.8, 28.7, 27.8, 26.3.

### 1-(1-(3-(4-(Benzo[d]thiazol-2-yl)piperidin-1-yl)propyl)-7-methoxy-1H-indol-3-yl)ethanone oxalate C669b

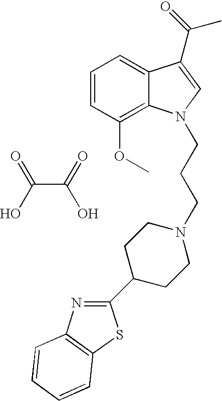

The title compound was synthesized according to TP12 at 0.35 mmol scale using 2-(piperidin-4-yl)benzo[d]thiazole (153 mg, 0.70 mmol) and no triethylamine. This gave 90 mg (58%) of the free base.

¹H NMR (400 MHz, CDCl₃) δ 7.98 (m, 2H), 7.86 (d, 1H, J=8.0 Hz), 7.71 (s, 1H), 7.45 (m, 1H), 7.35 (m, 1H), 7.17 (t, 1H, J=8 Hz), 6.71 (d, 1H J=7.8 Hz), 4.49 (t, 2H, J=6.6 Hz), 3.94 (s, 3H), 3.13 (m, 1H), 3.03 (m, 2H), 2.49 (s, 3H), 2.32 (t, 2H, J=6.8 Hz), 2.23-1.98 (m, 8H). The title compound (89 mg) was isolated as white crystals. MS (ES⁺, M+1)=448.

### 1-(1-(3-(4-(2-Chlorophenoxy)piperidin-1-yl)propyl)-7-methoxy-1H-indol-3-yl)ethanone oxalate C629b

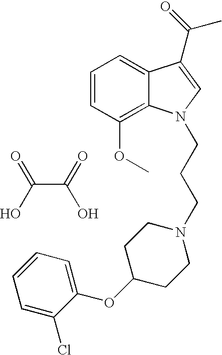

The title compound was synthesized according to TP12 at 0.35 mmol scale using 4-(2-chlorophenoxy)piperidine hydrochloride (174 mg, 0.70 mmol). This gave 105 mg (68%) of the free base.

¹H NMR (400 MHz, CDCl₃) δ 7.98 (d, 1H, J=8.0 Hz), 7.70 (s, 1H), 7.37 (dd, 1H, J=7.8, 1.6 Hz), 7.20 (m, 1H), 7.17 (t, H, J=8 Hz), 6.95 (dd, 1H, J=8.2, 1.4 Hz), 6.90 (dt, 1H, J=7.4, 1.4 Hz), 6.71 (d, 1H J=7.8 Hz), 4.47 (t, 2H, J=6.4 Hz), 4.40 (m, 1H), 3.94 (s, 3H), 2.73 (m, 1H), 2.49 (s, 3H), 2.32 (m, 4H), 2.05 (m, 4H), 1.90 (m, 2H). The title compound (96 mg) was isolated as white crystals. MS (ES⁺, M+1) 441.

### 1-(1-(3-(4-(4-Chlorophenoxy)piperidin-1-yl)propyl)-7-methoxy-1H-indol-3-yl)ethanone oxalate C645b

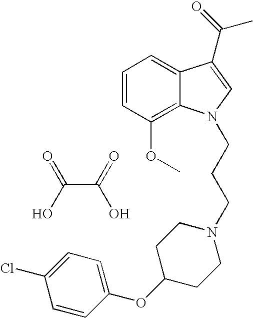

The title compound was synthesized according to TP12 at 0.35 mmol scale using 4-(4-chlorophenoxy)piperidine (115 mg, 0.55 mmol). This gave 60 mg (39%) of the free base. ¹H NMR (400 MHz, CDCl₃) δ 7.97 (d, 1H, J=8.0 Hz), 7.70 (s, 1H), 7.22 (m, 2H), 7.17 (t, 1H, J=8 Hz), 6.82 (m, 2H), 6.71 (d, 1H J=7.8 Hz), 4.47 (t, 2H, J=6.4 Hz), 4.29 (m, 1H), 3.94 (s, 3H), 2.71 (m, 1H), 2.49 (s, 3H), 2.32 (m, 4H), 2.05 (m, 4H), 1.82 (m, 2H). The title compound (60 mg) was isolated as white crystals. MS (ES⁺, M+1)=441.

### 1-(1-(3-(3α-(4-Chlorophenoxy)-8-azabicyclo[3.2.1]octan-8-yl)propyl)-7-methoxy-1H-indol-3-yl)ethanone oxalate C598b

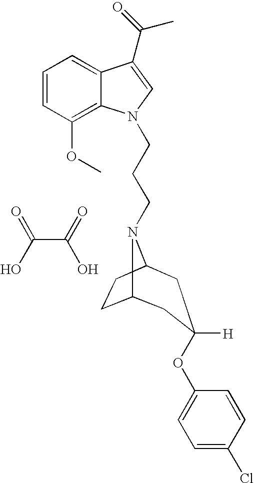

The title compound was synthesized according to TP12 at 0.35 mmol scale using 3-(4-chlorophenoxy)-8-azabicyclo[3.2.1]octane (166 mg, 0.7 mmol) and no triethylamine. This gave 115 mg (70%) of the free base.

¹H NMR (400 MHz, CDCl₃) δ 7.97 (d, 1H, J=8.0 Hz), 7.70 (s, 1H), 7.20 (m, 2H), 7.15 (t, 1H, J=8 Hz), 6.72 (m, 2H), 6.69 (d, 1H J=7.8 Hz), 4.49 (m 3H), 3.92 (s, 3H), 3.28 (m, 2H), 2.49 (s, 3H), 2.42 (t, 2H, J=7.2 Hz), 2.21 (m, 2H), 2.10-1.87 (m, 8H). The title compound (58 mg) was isolated as white crystals. MS (ES⁺, M)=467.

### 1-(7-bromo-1-(3-(4-(4-chlorophenoxy)piperidin-1-yl)propyl)-1H-indol-3-yl)ethanone oxalate C468b

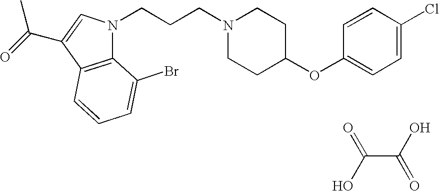

A 4 mL vial was charged with 1-(7-bromo-1-(3-chloropropyl)-1H-indol-3-yl)ethanone (139 mg, 0.44 mmol), 4-(4-chlorophenoxy)piperidine (87 mg, 0.41 mmol), Cs₂CO₃ (232 mg, 0.71 mmol), NaI (62 mg, 0.41 mmol) and CH₃CN (2 mL). The mixture was stirred at 50° C. overnight and then at 80° C. for 6 h. To the resulting suspension was added H₂O (1 mL), EtOAc (2 mL) and the organic layer was applied to a SCX ion exchange column. The cartridge washed with MeOH, and the crude product was eluded with NH₃ (MeOH). The resulting crude amine was purified by flash chromatography (EtOAc→EtOAc:MeOH 4:1) to give the free base (62 mg, 31%). Oxalic acid (1.1 eq) dissolved in acetone (0.3 ml) was added to the clear oil dissolved in acetone (0.5 ml). The precipitant was filtered off and dried to yield 62 mg of the title compound as white crystals.

¹H NMR (400 MHz, CDCl₃) (of free base) δ 8.43 (dd, J=8.0, 1.1 Hz, 1H), 7.83 (s, 1H), 7.45 (dd, J=7.7, 1.0 Hz, 1H), 7.24-7.20 (m, 2H), 7.12-7.08 (m, 1H), 6.84-6.80 (m, 2H), 4.65 (t, J=6.7 Hz, 2H), 4.32-4.26 (m, 1H), 2.76-2.68 (m, 2H), 2.50 (s, 3H), 2.36-2.27 (m, 4H), 2.15-1.98 (m, 4H), 1.89-1.79 (m, 2H).

¹³C NMR (100 MHz, CDCl₃) (of free base) δ 192.4, 155.9, 138.4, 133.1, 129.7, 129.4, 128.6, 125.7, 123.5, 122.1, 117.3, 116.3, 103.7, 72.7, 54.1, 50.3, 46.6, 30.7, 28.6, 27.5.

### 1-(3-(4-(benzo[d]thiazol-2-yl)piperidin-1-yl)propyl)-N-isobutyl-7-methoxy-1H-indole-3-carboxamide C184b

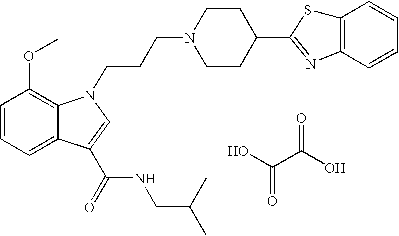

1-(3-chloropropyl)-N-isobutyl-7-methoxy-1H-indole-3-carboxamide (25 mg, 0.08 mmol), potassium iodide (2 mg, 0.01 mmol), DIPEA (21 mg, 0.16 mmol) and 2-(piperidin-4-yl)benzo[d]thiazole (34 mg, 0.16 mmol) were weighed into a vial and dry DMF (1 mL) was added. The vial was sealed and heated on a shaker at 80° C. for 24 h. The reaction mixture was diluted with EtOAc and washed with MgSO₄ (4% aq.), water and brine, dried over sodium sulphate, filtered and concentrated onto celite. The product was purified by flash chromatography 0-10% MeOH in DCM. Yield: 31 mg (78%).

¹H NMR (400 MHz, CDCl₃) δ: 7.96 (d, J=6.5 Hz, 1H), 7.85 (d, J=6.5 Hz, 1H), 7.54 (s, 1H), 7.49 (d, J=8.0 Hz, 1H), 7.45 (t, J=8.4 Hz, 1H), 7.34 (t, J=8.4 Hz, 1H), 7.12 (t, J=8.4 Hz, 1H), 6.67 (d, J=8.0 Hz, 1H), 4.45 (t, J=6.8 Hz, 2H), 3.94 (s, 3H), 3.32 (t, J=6.0 Hz, 1H), 3.11-2.98 (m, 3H), 2.33 (t, J=6.8 Hz, 1H), 2.07-1.90 (m, 9H), 1.00 (d, J=7.6 Hz, 1H).

¹³C NMR (400 MHz, CDCl₃) δ: 177.1, 166.4, 154.2, 148.9, 135.7, 133.9, 128.9, 127.2, 127.0, 125.8, 123.7, 123.0, 122.7, 113.6, 112.1, 104.2, 56.5, 56.2, 54.4, 49.1, 47.9, 42.7, 33.6, 30.0, 29.9, 21.4.

### 1-(7-methoxy-1-(3-(4-propoxypiperidin-1-yl)propyl)-1H-indol-3-yl)ethanone C730b

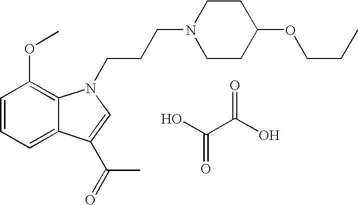

Prepared according to TP12 from 1-(1-(3-chloropropyl)-7-methoxy-1H-indol-3-yl)ethanone (199 mg. 0.75 mmol) and 4-propoxypiperidine (214 mg, 1.5 mmol). Yield of the title compound: 247 mg (89%).

¹H NMR (400 MHz, CDCl₃) δ: 7.97 (d, J=8.0 Hz, 1H), 7.694 (s, 1H), 7.161 (t, J=8.0 Hz, 1H), 6.70 (d, J=8.0 Hz, 1H), 4.44 (t, J=6.8 Hz, 2H), 3.92 (s, 3H), 3.396(t, J=6.8 Hz, 2H), 3.31-3.26 (m, 1H), 2.76-2.67 (m, 2H), 2.48 (s, 3H), 2.21 (t, J=6.4 Hz, 2H), 2.09-2.89 (m, 6H), 1.65-1.55 (m, 4H), 0.92 (t, J=7.2 Hz, 2H).

### 1-(7-methoxy-1-(3-(4-propoxypiperidin-1-yl)propyl)-1H-indol-3-yl)ethanol C902

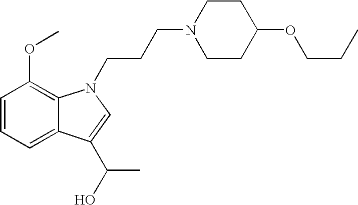

LiAlH₄ (3.8 mg, 0.1 mmol) was weighed into a dry, argon flushed vial, dry THF (1 mL) was added, followed by 1-(7-methoxy-1-(3-(4-propoxypiperidin-1-yl)propyl)-1H-indol-3-yl)ethanone (20 mg, 0.05 mmol) in THF (1 mL). The reaction mixture was left for 1 h at rt followed by 1 h at 80° C. Quenched with water and NaOH (2 N) and extracted with EtOAc. The combined organic phases was dried over Na₂SO₄ and concentrated in vacuo. The product was purified by flash chromatography 0-2% MeOH in DCM. Yield: 7 mg (50%).

¹H NMR (400 MHz, CDCl₃) δ: 7.32 (d, J=7.2 Hz, 1H), 7.01-6.97 (m, 2H), 6.61 (d, J=8.0 Hz, 1H), 5.17 (q, J=5.6 Hz, 1H), 4.35 (t, J=7.2 Hz, 2H), 3.90 (S, 1H), 3.37 (t, J=6.8 Hz, 1H), 3.29-3.24 (m, 1H), 2.74-2.70 (m, 2H), 2.26 (t J=6.8 Hz, 2H), 2.14-1.86 (m, 8H), 1.64-1.54 (m, 6H), 0.90 (t, J=7.2 Hz, 3H).

### 3-ethyl-7-methoxy-1-(3-(4-propoxypiperidin-1-yl)propyl)-1H-indole C903

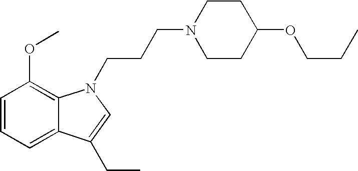

1-(7-methoxy-1-(3-(4-propoxypiperidin-1-yl)propyl)-1H-indol-3-yl)ethanone (20 mg, 0.05 mmol) was taken up in DCM in an argon flushed vial and cooled on an ice bath before slowly adding TiCl₄ (10 mg, 0.05 mmol, neat), after 10 min BH₃.NHMe₂ (6 mg, 0.1 mmol) was added in DCM (1 mL) and the reaction mixture was allowed to warm to rt, left for an additional 2 h before the reaction was quenched with HCl (2N). The crude product was taken up in EtOAc and washed with water, dried over Na₂SO₄ and concentrated in vacuo. The product was purified by prep TLC from 2% MeOH in DCM. Yield: 0.9 mg (8%). UV/MS: 95/90.

### N-(3-chlorobenzyl)-7-methoxy-1-(3-((1R,3r,5S)-3-(2-oxo-2-phenylethyl)-8-azabicyclo[3.2.1]octan-8-yl)propyl)-1H-indole-3-carboxamide C221b

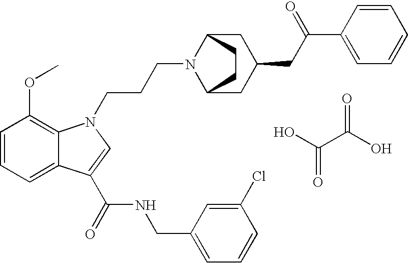

Prepared according to TP12 from N-(3-chlorobenzyl)-1-(3-chloropropyl)-7-methoxy-1H-indole-3-carboxamide (98 mg. 0.25 mmol) and 2-((1R,3r,5S)-8-azabicyclo[3.2.1]octan-3-yl)-1-phenylethanone (115 mg, 0.5 mmol). Yield of the title compound: 113 mg (77%).

¹H-NMR (400 MHz, CDCl₃) δ: 7.91-7.89 (m, 2H), 7.72 (d, J=0.8 Hz, 1H), 7.64 (d, J=8.0 Hz, 1H), 7.57-7.53 (m, 1H), 7.46-7.43 (m, 2H), 7.34 (s, 1H), 7.26-7.19 (m, 3H), 7.10 (dt, J=1.2 and 8.0 Hz, 1H), 6.65 (d, J=8.0 Hz, 1H), 4.64 (d, J=6.0 Hz, 2H), 4.47 (t, J=6.8 Hz, 2H), 3.92 (s, 3H), 3.17-3.12 (m, 2H), 3.05 (d, J=7.2 Hz, 2H), 2.56-2.49 (m, 1H), 2.22-2.15 (m, 4H), 1.99-1.87 (m, 4H), 1.68-1.57 (m, 2H), 1.25-1.17 (m, 2H).

### 7-methoxy-N-(3-methylbenzyl)-1-(3-((1R,3r,5S)-3-(2-oxo-2-phenylethyl-8-azabicyclo[3.2.1]octan-8-yl)propyl)-1H-indole-3-carboxamide C193b

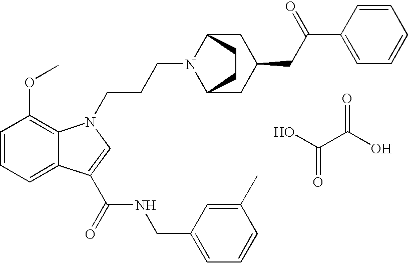

Prepared according to TP12 from 1-(3-chloropropyl)-7-methoxy-N-(3-methylbenzyl)-1H-indole-3-carboxamide (98 mg. 0.25 mmol) and 2-((1R,3r,5S)-8-azabicyclo[3.2.1]octan-3-yl)-1-phenylethanone (115 mg, 0.5 mmol). Yield of the title compound: 111 mg (46%).

¹H NMR (400 MHz, CDCl₃) δ: 7.93-7.89 (m, 2H), 7.75-7.53 (m, 3H), 7.46-7.40 (m, 2H), 7.33-7.06 (m, 5H), 4.68-4.63 (m, 2H), 4.50-4.43 (m, 2H), 3.97 (s, 3H), 3.17-3.05 (m, 4H), 2.53-2.45 (m, 1H), 2.33 (s, 3H), 2.27-2.15 (m, 4H), 1.98-1.90 (m, 4H), 1.67-1.60 (m, 2H), 1.22-1.17 (m, 2H).

¹³C NMR (400 MHz, CDCl₃) δ: 200.3, 147.8, 138.9, 138.5, 133.3, 133.2, 132.8, 131.1, 129.0, 129.0, 128.8, 128.7, 128.7, 128.2, 126.3, 124.9, 122.1, 113.1, 103.4, 58.3, 55.5, 50.6, 50.6, 48.1, 46.9, 43.6, 35.1, 30.2, 27.1, 23.9.

Synthesis of Libraries

Alkyl halides (0.03 mmol/reaction) were dissolved in DMF (0.5 mL/reaction). Secondary amines (0.06 mmol/reaction) and DIEA (0.06 mmol/reaction) were dissolved in DMF (0.3 mL/reaction). NaI (cat) was added to a microtiter plate. The microtiter plate was moved to liquid handler and the solutions containing alkyl halides and amines were dispensed. The microtiter plate was shaken at 80° C. for 22 hours. Volatile materials were then removed at reduced pressure. The remaining crude products were dissolved in DMF (0.32 mL) and filtered using a 96-position filter plate (0.8 mL, 0.4 micrometer) into microtiter plates. The crude products were purified by preparative LC/MS according to PP (analytical methods). Purity analyses of the purified products were performed according to AP (analytical methods).

The following compounds were prepared using the library procedure:

### N-(3,4-dichlorobenzyl)-7-isopropoxy-1-(3-(4-(3-phenoxypropyl)-1,4-diazepan-1-yl)propyl)-1H-indole-3-carboxamide C001

Amount made: 1.6 mg. LCMS m/z 651 [M+H]⁺, purity (UV/MS) 97/80.

### 1-(3-(4-(2-(4-chloronaphthalen-1-yloxy)ethyl)piperazin-1-yl)propyl)-N-(3-fluorobenzyl)-7-methoxy-1H-indole-3-carboxamide C002

Amount made: 3.3 mg. LCMS m/z 629 [M+H]⁺, purity (UV/MS) 100/90.

### N-(3,4-dichlorobenzyl)-7-ethyl-1-(3-(4-(2-phenoxyethyl)piperazin-1-yl)propyl)-1H-indole-3-carboxamide C003

Amount made: 3.3 mg. LCMS m/z 593 [M+H]⁺, purity (UV/MS) 100/90.

### N-(3,4-dichlorobenzyl)-7-ethyl-1-(3-(4-phenethyl-1,4-diazepan-1-yl)propyl)-1H-indole-3-carboxamide C004

Amount made: 6.3 mg. LCMS m/z 591 [M+H]⁺, purity (UV/MS) 96/80.

### N-(3,4-dichlorobenzyl)-7-ethyl-1-(3-(4-(4-fluorobenzyl)-1,4-diazepan-1-yl)propyl)-1H-indole-3-carboxamide C005

Amount made: 5.6 mg. LCMS m/z 595 [M+H]⁺, purity (UV/MS) 95/70.

### 1-(3-((1R,3r,5S)-3-(2-(3,4-dichlorophenyl)-2-oxoethyl)-8-azabicyclo[3.2.1]octan-8-yl)propyl)-7-isopropoxy-N-(3-methylbenzyl)-1H-indole-3-carboxamide C006

Amount made: 1.1 mg. LCMS m/z 660 [M+H]⁺, purity (UV/MS) 100/90.

### 1-(3-((1R,3r,5S)-3-(2-(4-chlorophenly)-2-oxoethyl)-8-azabicyclo[3.2.1]octan-8-yl)propyl)-7-isopropoxy-N-(3-methylbenzyl)-1H-indole-3-carboxamide C007

Amount made: 5.0 mg. LCMS m/z 626 [M+H]⁺, purity (UV/MS) 99/80.

### 7-isopropoxy-N-(3-methylbenzyl)-1-(3-((1R,3r,5S)-3-(2-oxo-2-(pyridin-2-yl)ethyl)-8-azabicyclo[3.2.1]octan-8-propyl)-1H-indole-3-carboxamide C008

Amount made: 1.8 mg. LCMS m/z 593 [M+H]⁺, purity (UV/MS) 100/70.

### 1-(3-(4-(2-chloro-6-fluorobenzyl)-1,4-diazepan-1-yl)propyl)-N-(3,4-dichlorobenzyl)-7-ethyl-1H-indole-3-carboxamide C009

Amount made: 7.3 mg. LCMS m/z 629 [M+H]⁺, purity (UV/MS) 98/80.

### 1-(3-((1R,3r,5S)-3-(2-(3-chloro-4-fluorophenyl)-2-oxoethyl)-8-azabicyclo[3.2.1]octan-8-yl)propyl)-7-isopropoxy-N-(3-methylbenzyl)-1H-indole-3-carboxamide C010

Amount made: 3.3 mg. LCMS m/z 644 [M+H]⁺, purity (UV/MS) 97/70.

### 7-isopropoxy-N-(3-methylbenzyl)-1-(3-(4-(2-phenoxyethyl)piperidin-1-yl)propyl)-1H-indole-3-carboxamide C011

Amount made: 0.9 mg. LCMS m/z 568 [M+H]⁺, purity (UV/MS) 73/60.

### 1-(3-(4-(2-(4-fluorophenoxy)ethyl)-1,4-diazepan-1-yl)propyl)-7-isopropoxy-N-(3-methylbenzyl)-1H-indole-3-carboxamide C012

Amount made: 1.4 mg. LCMS m/z 601 [M+H]⁺, purity (UV/MS) 100/100.

### 1-(3-((1S,4S)-5-(2-(4-fluorophenoxy)ethyl)-2,5-diazabicyclo[2.2.2]octan-2-yl)propyl)-7-isopropoxy-N-(3-methylbenzyl)-1H-indole-3-carboxamide C013

Amount made: 6.0 mg. LCMS m/z/z 613 [M+H]⁺, purity (UV/MS) 100/90.

### N-(3,4-dichlorobenzyl)-1-(3-((1R,5S)-3-(2-(4-fluorophenoxy)ethyl)-3,8-diazabicyclo[3.2.1]octan-8-yl)propyl)-7-isopropoxy-1H-indole-3-carboxamide C014

Amount made: 2.6 mg. LCMS m/z 667 [M+H]⁺, purity (UV/MS) 90/70. N-benzyl-7-ethyl-1-(3-(4-(2-phenoxyethyl)-1,4-diazepan-1-yl)propyl)-1H-indole-3-carboxamide C015

Amount made: 1.5 mg. LCMS m/z 539 [M+H]⁺, purity (UV/MS) 100/100.

### N-(3,4-dichlorobenzyl)-1-(3-((1R,3r,5S)-3-(2-(3,4-dichlorophenyl)-2-oxoethyl)-8-azabicyclo[3.2.1]octan-8-yl)propyl)-7-isopropoxy-1H-indole-3-carboxamide C016

Amount made: 2.3 mg. LCMS m/z 714 [M+H]⁺, purity (UV/MS) 96/60.

### 1-(3-((1R,3r,5S)-3-(2-(4-chlorophenyl)-2-oxoethyl)-8-azabicyclo[3.2.1]octan-8-yl)propyl)-N-(3,4-dichlorobenzyl)-7-isopropoxy-1H-indole-3-carboxamide C017

Amount made: 2.1 mg. LCMS m/z 680 [M+H]⁺, purity (UV/MS) 98/70.

### 1-(3-((1R,3r,5S)-3-(2-(3-chloro-4-fluorophenyl)-2-oxoethyl)-8-azabicyclo[3.2.1]octan-8-yl)propyl)-N-(3,4-dichlorobenzyl)-7-isopropoxy-1H-indole-3-carboxamide C018

Amount made: 6.6 mg. LCMS m/z 698 [M+H]⁺, purity (UV/MS) 87/70.

### N-(3,4-dichlorobenzyl)-7-isopropoxy-1-(3-(4-(2-phenoxyethyl)piperidin-1-yl)propyl)-1H-indole-3-carboxamide C019

Amount made: 3.9 mg. LCMS m/z 622 [M+H]⁺, purity (UV/MS) 85/50.

### 1-(3-(4-(2-(4-chloronaphthalen-1-yloxy)ethyl)piperazin-1-yl)propyl)-N-(3,4-dichlorobenzyl)-7-ethyl-1H-indole-3-carboxamide C020

Amount made: 6.8 mg. LCMS m/z 677 [M+H]⁺, purity (UV/MS) 100/80.

### N-(3,4-dichlorobenzyl)-1-(3-(4-(2-(4-fluorophenoxy ethyl)-1,4-diazepan-1-yl)propyl)-7-isopropoxy-1H-indole-3-carboxamide C021

Amount made: 3.2 mg. LCMS m/z 655 [M+H]⁺, purity (UV/MS) 100/90.

### N-(3,4-dichlorobenzyl)-7-isopropoxyl)-1-(3-(4-(2-phenoxyethyl)-1,4-diazepan-1-yl)propyl)-1H-indole-3-carboxamide C022

Amount made: 1.1 mg. LCMS m/z 637 [M+H]⁺, purity (UV/MS) 100/100.

### 1-(3-((1S,4S)-5-(2-(4-fluorophenoxy)ethyl)-2,5-diazabicyclo[2.2.2]octan-2-yl)propyl)-7-methoxy-N-(3-methylbenzyl)-1H-indole-3-carboxamide C023

Amount made: 2.5 mg. LCMS m/z 585 [M+H]⁺, purity (UV/MS) 100/90.

### 1-(3-((1R,5S)-3-(2-(4-fluorophenoxy)ethyl)-3,8-diazabicyclo[3.2.1]octan-8-yl)propyl)-7-methoxy-N-(3-methylbenzyl)-1H-indole-3-carboxamide C024

Amount made: 2.1 mg. LCMS m/z 585 [M+H]⁺, purity (UV/MS) 100/80.

### N-(3,4-dichlorobenzyl)-7-ethyl-1-(3-(4-(3-phenoxypropyl)piperazin-1-yl)propyl)-1H-indole-3-carboxamide C025

Amount made: 8.2 mg. LCMS m/z 607 [M+H]⁺, purity (UV/MS) 99/50.

### 7-methoxy-N-(3-methylbenzyl)-1-(3-(4-(3-phenoxypropyl)-1,4-diazepan-1-yl)propyl)-1H-indole-3-carboxamide C026

Amount made: 1.2 mg. LCMS m/z 569 [M+H]⁺, purity (UV/MS) 98/90.

### 1-(3-(C1R,3r,5S)-3-(2-(4-chlorophenyl)-2-oxoethyl)-8-azabicyclo[3.2.1]octan-8-yl)propyl)-7-methoxy-N-(3-methylbenzyl)-1H-indole-3-carboxamide C027

Amount made: 3.1 mg. LCMS m/z 598 [M+H]⁺, purity (UV/MS) 99/70.

### N-(3,4-dichlorobenzyl)-1-(3-((1S,4S)-5-(2-(4-fluorophenoxy)ethyl)-2,5-diazabicyclo[2.2.2]octan-2-yl)propyl)-7-isopropoxy-1H-indole-3-carboxamide C028

Amount made: 3.2 mg. LCMS m/z 667 [M+H]⁺, purity (UV/MS) 100/90.

### 1-(3-(4-(4-fluorobenzyl)-1,4-diazepan-1-yl)propyl)-7-methoxy-N-(2-methylbenzyl)-1H-indole-3-carboxamide C029

Amount made: 0.3 mg, LCMS m/z 543 [M+H]⁺, purity (UV/MS) 99/90.

### 1-(3-(4-(2-(4-chlorophenoxy)ethyl)piperazin-1-yl)propyl)-7-methoxy-N-(2-methylbenzyl)-1H-indole-3-carboxamide C030

Amount made: 6.3 mg. LCMS m/z 575 [M+H]⁺, purity (UV/IS) 100/100.

### N-(3,4-dichlorobenzyl)-7-ethyl-1-(3-((1S,4S)-5-(2-(4-fluorophenoxy)ethyl)-2,5-diazabicyclo[2.2.2]octan-2-yl)propyl)-1H-indole-3-carboxamide C031

Amount made: 1.6 mg. LCMS m/z 637 [M+H]⁺, purity (UV/MS) 100/100.

### N-(3,4-dichlorobenzyl)-7-ethyl-1-(3-((1R,5S)-3-(2-(4-fluorophenoxy)ethyl)-3,8-diazabicyclo[3.2.1]octan-8-yl)propyl)-1H-indole-3-carboxamide C032

Amount made: 2.6 mg. LCMS m/z 637 [M+H]⁺, purity (UV/MS) 100/100.

### 7-isopropoxy-N-(3-methylbenzyl)-1-(3-(4-(3-phenoxypropyl)-1,4-diazepan-1-yl)propyl)-1H-indole-3-carboxamide C033

Amount made: 2.5 mg. LCMS m/z 597 [M+H]⁺, purity (UV/MS) 100/90.

### 1-(3-((1R,3r,5S)-3-(2-(4-chlorophenyl)-2-oxoethyl)-8-azabicyclo[3.2.1]octan-8-yl)propyl)-N-(3,4-dichlorobenzyl)-7-ethyl-1H-indole-3-carboxamide C034

Amount made: 4.0 mg. LCMS m/z 650 [M+H]⁺, purity (UV/MS) 100/80.

### N-benzyl-1-(3-(1-benzylpyrrolidin-3-ylamino)propyl)-7-ethyl-1H-indole-3-carboxamide C035

Amount made: 5.2 mg. LCMS m/z 495 [M+H]⁺, purity (UV/MS) 98/60.

### N-(3,4-dichlorobenzyl)-7-ethyl-1-(3-(4-(2-phenoxyethyl)piperidin-1-yl)propyl)-1H-indole-3-carboxamide C036

Amount made: 4.9 mg. LCMS m/z 592 [M+H]⁺, purity (UV/MS) 100/80.

### 1-(3-(4-(2-(diisopropylamino)ethyl)piperazin-1-yl)propyl)-7-methoxy-N-(2-methylbenzyl) 1H-indole-3-carboxamide C037

Amount made: 8.3 mg. LCMS m/z 548 [M+H]⁺, purity (UV/MS) 98/80.

### N-(3,4-dichlorobenzyl)-7-ethyl-1-(3-(4-(2-(4-fluorophenoxy)ethyl)-1,4-diazepan-1-yl)propyl)-1H-indole-3-carboxamide C038

Amount made: 5.2 mg. LCMS m/z 625 [M+H]⁺, purity (UV/MS) 97/80.

### N-(3,4-dichlorobenzyl)-7-ethyl-1-(3-(4-(2-phenoxyethyl)-4-diazepan-1-yl)propyl)-1H-indole-3-carboxamide C039

Amount made: 2.0 mg. LCMS m/z 607 [M+H]⁺, purity (UV/MS) 100/100.

### 7-methoxy-N-(2-methylbenzyl)-1-(3-(4-(2-morpholino-2-oxoethyl)piperazin-1-yl)propyl)-1-indole-3-carboxamide C040

Amount made: 5.1 mg. LCMS m/z 548 [M+H]⁺, purity (UV/MS) 100/80.

### 1-(3-((1R,5S)-3-(2-(4-fluorophenoxy)ethyl)-3,8-diazabicyclo[3.2.1]octan-8-yl)propyl)-7-isopropoxy-N-(3-methylbenzyl)-1H-indole-3-carboxamide C041

Amount made: 3.2 mg. LCMS m/z 613 [M+H]⁺, purity (UV/MS) 85/50.

### 7-methoxy-N-(2-methylbenzyl)-1-(3-(4-phenethyl-1,4-diazepan-1-yl)propyl)-1H-indole-3-carboxamide C042

Amount made: 5.9 mg. LCMS m/z 539 [M+H]⁺, purity (UV/MS) 99/70.

### 7-methoxy-N-(3-methylbenzyl)-1-(3-(4-(2-phenoxyethyl)piperidin-1-yl)propyl)-1H-indole-3-carboxamide C043

Amount made: 1.9 mg. LCMS m/z 540 [M+H]⁺, purity (UV/MS) 100/70.

### N-benzyl-7-ethyl-1-(3-((1S,4S)-5-(2-(4-fluorophenoxy)ethyl)-2,5-diazabicyclo[2.2.2]octan-2-yl)propyl)-1H-indole-3-carboxamide C044

Amount made: 1.5 mg. LCMS m/z 569 [M+H]⁺, purity (UV/MS) 100/100.

### N-benzyl-7-ethyl-1-(3-((1R,5S)-3-(2-(4-fluorophenoxy)ethyl)-3,8-diazabicyclo[3.2.1]octan-8-yl)propyl)-1H-indole-3-carboxamide C045

Amount made: 4.2 mg. LCMS m/z 569 [M+H]⁺, purity (UV/MS) 100/100.

### 1-(3-(4-(2-chloro-6-fluorobenzyl)-1,4-diazepan-1-yl)propyl)-7-methoxy-N-(2-methylbenzyl)-1H-indole-3-carboxamide C046

Amount made: 4.1 mg. LCMS m/z 577 [M+H]⁺, purity (UV/MS) 98/70.

### 1-(3-(4-(2-chloro-6-fluorobenzyl)-1,4-diazepan-1-yl)propyl)-N-(3-fluorobenzyl)-7-methoxy-1H-indole-3-carboxamide C047

Amount made: 4.1 mg. LCMS m/z 581 [M+H]⁺, purity (UV/MS) 97/70.

### 7-methoxy-N-(2-methylbenzyl)-1-(3-(4-(3-phenoxypropyl)piperazin-1-yl)propyl)-1H-indole-3-carboxamide C048

Amount made: 3.1 mg. LCMS m/z 555 [M+H]⁺, purity (UV/MS) 100/90.

### 7-methoxy-N-(2-methylbenzyl)-1-(3-(4-phenethylpiperazin-1-yl)propyl)-1H-indole-3-carboxamide C049

Amount made: 6.2 mg. LCMS m/z 525 [M+H]⁺, purity (UV/MS) 100/90.

### 1-(3-(1-benzylpyrrolidin-3-ylamino)propyl)-N-(3,4-dichlorobenzyl)-7-ethyl-1H-indole-3-carboxamide C050

Amount made: 1.0 mg. LCMS m/z 563 [M+H]⁺, purity (UV/MS) 97/80.

### N-benzyl-7-ethyl-1-(3-(4-(2-phenoxyethyl)piperidin-1-yl)propyl)-1H-indole-3-carboxamide C051

Amount made: 1.8 mg. LCMS m/z 524 [M+H]⁺, purity (UV/MS) 100/100.

### N-benzyl-7-ethyl-1-(3-(4-(2-(4-fluorophenoxy)ethyl)-1,4-diazepan-1-yl)propyl)-1H-indole-3-carboxamide C052

Amount made: 4.8 mg. LCMS m/z 557 [M+H]⁺, purity (UV/MS) 96/80.

### N-(3-fluorobenzyl)-1-(3-(4-(4-fluorobenzyl)-1,4-diazepan-1-yl)propyl)-7-methoxy-1H-indole-3-carboxamide C053

Amount made: 1.0 mg. LCMS m/z 547 [M+H]⁺, purity (UV/MS) 99/90.

### N-(3,4-dichlorobenzyl)-1-(3-(4-(2-(diisopropylamino)ethyl)piperazin-1-yl)propyl)-7-ethyl-1H-indole-3-carboxamide C054

Amount made: 7.2 mg. LCMS m/z 600 [M+H]⁺, purity (UV/MS) 100/90.

### N-(3,4-dichlorobenzyl)-7-ethyl-1-(3-(4-(2-morpholino-2-oxoethyl)piperazin-1-yl)propyl)-1H-indole-3-carboxamide C055

Amount made: 5.9 mg. LCMS m/z 600 [M+H]⁺, purity (UV/MS) 100/100.

### 7-methoxy-N-(2-methylbenzyl)-1-(3-(4-(2-phenoxyethyl)piperazin-1-yl)propyl)-1H-indole-3-carboxamide C056

Amount made: 1.9 mg. LCMS m/z 541 [M+H]⁺, purity (UV/MS) 100/100.

### 1-(3-(4-(2-(diisopropylamino)ethyl)piperazin-1-yl)propyl)-7-isopropoxy-N-(3-methylbenzyl)-1H-indole-3-carboxamide C057

Amount made: 7.1 mg. LCMS m/z 576 [M+H]⁺, purity (UV/MS) 98/70.

### N-(3,4-dichlorobenzyl)-7-isopropoxy-1-(3-(4-phenethyl-1,4-diazepan-1-yl)propyl)-1H-indole-3-carboxamide C058

Amount made: 5.3 mg. LCMS m/z 621 [M+H]⁺, purity (UV/MS) 99/80.

### N-(3,4-dichlorobenzyl)-7-isopropoxy-1-(3-(4-(2-phenoxyethyl)piperazin-1-yl)propyl)-1H-indole-3-carboxamide C059

Amount made: 4.0 mg. LCMS m/z 623 [M+H]⁺, purity (UV/MS) 100/90.

### N-(3,4-dichlorobenzyl)-7-isopropoxy-1-(3-(4-(2-morpholino-2-oxoethyl)piperazin-1-yl)propyl)-1H-indole-3-carboxamide C060

Amount made: 2.3 mg. LCMS m/z 630 [M+H]⁺, purity (UV/MS) 100/90.

### N-(3,4-dichlorobenzyl)-1-(3-(4-(2-(diisopropylamino)ethyl)piperazin-1-yl)propyl)-7-isopropoxy-1H-indole-3-carboxamide C061

Amount made: 3.9 mg. LCMS m/z 630 [M+H]⁺, purity (UV/MS) 97/80.

### 1-(3-(4-(2-(4-chlorophenoxy)ethyl)piperazin-1-yl)propyl)-N-(3,4-dichlorobenzyl)-7-isopropoxy-1H-indole-3-carboxamide C062

Amount made: 6.4 mg. LCMS m/z 657 [M+H]⁺, purity (UV/MS) 100/100.

### 1-(3-(1-benzylpyrrolidin-3-ylamino)propyl)-N-(3,4-dichlorobenzyl)-7-isopropoxy-1H-indole-3-carboxamide C063

Amount made: 2.6 mg. LCMS m/z 593 [M+H]⁺, purity (UV/MS) 94/80.

### 1-(3-(dihydro-1H-pyrido[1,2-a]pyrazin-2(6H,7H,8H,9H,9aH)-yl)propyl)-7-isopropoxy-N-(3-methylbenzyl)-1H-indole-3-carboxamide C064

Amount made: 2.9 mg. LCMS m/z 503 [M+H]⁺, purity (UV/MS) 100/100.

### 7-isopropoxy-N-(3-methylbenzyl)-1-(3-(4-phenethylpiperazin-1-yl)propyl)-1H-indole-3-carboxamide C065

Amount made: 4.6 mg. LCMS m/z 553 [M+H]⁺, purity (UV/MS) 85/60.

### 7-isopropoxy-N-(3-methylbenzyl)-1-(3-(4-(3-phenoxypropyl)piperazin-1-yl)propyl)-1H-indole-3-carboxamide C066

Amount made: 3.0 mg. LCMS m/z 583 [M+H]⁺, purity (UV/MS) 99/70.

### 1-(3-(4-(2-(4-chloronaphthalen-1-yloxy)ethyl)piperazin-1-yl)propyl 7-isopropoxy-N-(3-methylbenzyl)-1H-indole-3-carboxamide C067

Amount made: 2.9 mg. LCMS m/z 653 [M+H]⁺, purity (UV/MS) 100/90.

### 7-isopropoxy-N-(3-methylbenzyl)-1-(3-(4-phenethyl-1,4-diazepan-1-yl)propyl)-1H-indole-3-carboxamide C068

Amount made: 1.5 mg. LCMS m/z 567 [M+H]⁺, purity (UV/MS) 99/70.

### 7-methoxy-N-(3-methylbenzyl)-1-(3-((1R,3r,5S)-3-(2-oxo-2-(pyridin-2-yl)ethyl)-8-azabicyclo[3.2.1]octan-8-yl)propyl)-1H-indole-3-carboxamide C069

Amount made: 1.3 mg. LCMS m/z 565 [M+H]⁺, purity (UV/MS) 97/80.

### 7-isopropoxy-N-(3-methylbenzyl)-1-(3-(4-(2-morpholino-2-oxoethyl)piperazin-1-yl)propyl)-1H-indole-3-carboxamide C070

Amount made: 5.8 mg. LCMS m/z 576 [M+H]⁺, purity (UV/MS) 100/80.

### N-(3,4-dichlorobenzyl)-7-isopropoxy-1-(3-(4-phenethylpiperazin-1-yl)propyl)-1H-indole-3-carboxamide C071

Amount made: 3.6 mg. LCMS m/z 607 [M+H]⁺, purity (UV/MS) 100/80.

### 1-(3-(4-(2-(4-chlorophenoxy)ethyl)piperazin-1-yl)propyl)-7-isopropoxy-N-(3-methylbenzyl)-1H-indole-3-carboxamide C072

Amount made: 3.1 mg. LCMS m/z 603 [M+H]⁺, purity (UV/MS) 100/90.

### 1-(3-(1-benzylpyrrolidin-3-ylamino)propyl)-7-isopropoxy-N-(3-methylbenzyl)-1H-indole-3-carboxamide C073

Amount made: 3.5 mg. LCMS m/z 539 [M+H]⁺, purity (UV/MS) 98/80.

### N-benzyl-1-(3-(dihydro-1H-pyrido[1,2-a]pyrazin-2(6H,7H,8H,9H,9aH)-yl)propyl)-7-ethyl-1H-indole-3-carboxamide C074

Amount made: 3.9 mg. LCMS m/z 459 [M+H]⁺, purity (UV/MS) 100/100.

### N-benzyl-7-ethyl-1-(3-(4-(3-phenoxypropyl)piperazin-1-yl)propyl)-1H-indole-3-carboxamide C075

Amount made: 2.2 mg. LCMS m/z 539 [M+H]⁺, purity (UV/MS) 100/100.

### 1-(3-(4-(2-(diisopropylamino)ethyl)piperazin-1-yl)propyl)-N-(3-fluorobenzyl)-7-methoxy-1H-indole-3-carboxamide C076

Amount made: 9.0 mg. LCMS m/z 552 [M+H]⁺, purity (UV/MS) 81/60.

### N-benzyl-1-(3-(4-(2-chloro-6-fluorobenzyl)-1,4-diazepan-1-yl)propyl)-7-ethyl-1H-indole-3-carboxamide C077

Amount made: 3.6 mg. LCMS m/z 561 [M+H]⁺, purity (UV/MS) 100/90.

### N-benzyl-7-ethyl-1-(3-(4-(4-fluorobenzyl)-1,4-diazepan-1-yl)propyl)-1H-indole-3-carboxamide C078

Amount made: 5.1 mg. LCMS m/z 527 [M+H]⁺, purity (UV/MS) 90/60.

### N-(3-fluorobenzyl)-7-methoxy-1-(3-(4-(2-morpholino-2-oxoethyl)piperazin-1-yl)propyl)-1H-indole-3-carboxamide C079

Amount made: 9.4 mg. LCMS m/z 552 [M+H]⁺, purity (UV/MS) 98/80.

### N-(3-fluorobenzyl)-7-methoxy-1-(3-(4-(2-phenoxyethyl)piperazin-1-yl)propyl)-1H-indole-3-carboxamide C080

Amount made: 6.3 mg. LCMS m/z 545 [M+H]⁺, purity (UV/MS) 100/90.

### N-(3-fluorobenzyl)-7-methoxy-1-(3-(4-phenethyl-1,4-diazepan-1-yl)propyl)-1H-indole-3-carboxamide C081

Amount made: 6.0 mg. LCMS m/z 543 [M+H]⁺, purity (UV/MS) 98/80.

### N-benzyl-1-(3-(4-(2-(diisopropylamino)ethyl)piperazin-1-yl)propyl)-7-ethyl-1H-indole-3-carboxamide C082

Amount made: 6.7 mg. LCMS m/z 532 [M+H]⁺, purity (UV/MS) 99/80.

### N-benzyl-1-(3-(4-(2-(4-chlorophenoxy)ethyl)piperazin-1-yl)propyl)-7-ethyl-1H-indole-3-carboxamide C083

Amount made: 8.9 mg. LCMS m/z 559 [M+H]⁺, purity (UV/MS) 100/90.

### 7-isopropoxy-N-(3-methylbenzyl)-1-(3-(4-(2-phenoxyethyl)piperazin-1-yl)propyl)-1H-indole-3-carboxamide C084

Amount made: 4.8 mg. LCMS m/z 569 [M+H]⁺, purity (UV/MS) 100/90.

### N-(3,4-dichlorobenzyl)-7-methoxy-1-(3-(4-phenethylpiperazin-1-yl)propyl)-1H-indole-3-carboxamide C085

Amount made, 2.1 mg. LCMS m/z 579 [M+H]⁺, purity (UV/MS) 100/100. N-(3,4-dichlorobenzyl)-7-ethyl-1-(3-(4-(3-phenoxypropyl)-1,4-diazepan-1-yl)propyl)-1H-indole-3-carboxamide C086

Amount made: 1.8 mg. LCMS m/z 621 [M+H]⁺, purity (UV/MS) 100/90.

### 1-(3-(4-(2-(4-fluorophenoxy)ethyl 1,4-diazepan-1-yl)propyl)-7-methoxy-N-(3-methylbenzyl)-1H-indole-3-carboxamide C087

Amount made: 2.4 mg. LCMS m/z 573 [M+H]⁺, purity (UV/MS) 100/90.

### 7-methoxy-N-(3-methylbenzyl)-1-(3-(4-(2-phenoxyethyl)-1,4-diazepan)-1-yl)propyl)-1H-indole-3-carboxamide C088

Amount made: 1.0 mg. LCMS m/z 555 [M+H]⁺, purity (UV/MS) 100/90.

### 1-(3-(1-benzylpyrrolidin-3-ylamino)propyl)-N-(3,4-dichlorobenzyl)-7-methoxy-1H-indole-3-carboxamide C089

Amount made: 3.4 mg. LCMS m/z 565 [M+H]⁺, purity (UV/MS) 99/80.

### 1-(3-(4-(2-(4-chlorophenoxy)ethyl)piperazin-1-yl)propyl)-N-(3,4-dichlorobenzyl)-7-methoxy-1H-indole-3-carboxamide C090

Amount made: 8.1 mg. LCMS m/z 629 [M+H]⁺, purity (UV/MS) 100/90.

### N-(3,4-dichlorobenzyl)-1-(3-(4-(2-(diisopropylamino)ethyl)piperazin-1-yl)propyl)-7-methoxy-1H-indole-3-carboxamide C091

Amount made: 6.9 mg. LCMS m/z 602 [M+H]⁺, purity (UV/MS) 99/90.

### N-(3,4-dichlorobenzyl)-7-methoxy-1-(3-(4-(2-morpholino-2-oxoethyl)piperazin-1-yl)propyl)-1H-indole-3-carboxamide C092

Amount made: 8.5 mg. LCMS m/z 602 [M+H]⁺, purity (UV/MS) 100/80.

### N-(3,4-dichlorobenzyl)-7-methoxy-1-(3-(4-(2-phenoxyethyl)piperazin-1-yl)propyl)-1H-indole-3-carboxamide C093

Amount made: 6.9 mg. LCMS m/z 595 [M+H]⁺, purity (UV/MS) 100/90.

### N-(3,4-dichlorobenzyl)-7-methoxy-1-(3-(4-phenethyl-1,4-diazepan-1-yl)propyl)-1H-indole-3-carboxamide C094

Amount made: 8.0 mg. LCMS m/z 593 [M+H]⁺, purity (UV/MS) 99/80.

### N-(3,4-dichlorobenzyl)-1-(3-(4-(4-fluorobenzyl)-1,4-diazepan-1-yl)propyl)-7-methoxy-1H-indole-3-carboxamide C095

Amount made: 1.7 mg. LCMS m/z 597 [M+H]⁺, purity (UV/MS) 99/90.

### 1-(3-(4-(2-chloro-6-fluorobenzyl)-1,4-diazepan-1-yl)propyl)-N-(3,4-dichlorobenzyl)-7-methoxy-1H-indole-3-carboxamide C096

Amount made: 4.7 mg. LCMS m/z 631 [M+H]⁺, purity (UV/MS) 89/60.

### 1-(3-(4-(2-(4-chloronaphthalen-1-yloxy)ethyl)piperazin-1-yl)propyl)-N-(3,4-dichlorobenzyl)-7-methoxy-1H-indole-3-carboxamide C097

Amount made: 4.0 mg. LCMS m/z 679 [M+H]⁺, purity (UV/MS) 100/90.

### 1-(3-(4-(2-(4-chloronaphthalen-1-yloxy)ethyl)piperazin-1-yl)propyl)-N-(3,4-dichlorobenzyl)-7-isopropoxy-1H-indole-3-carboxamide C098

Amount made: 4.3 mg. LCMS m/z 707 [M+H]⁺, purity (UV/MS) 100/90.

### N-(3,4-dichlorobenzyl)-7-ethyl-1-(3-(4-phenethylpiperazin-1-yl)propyl)-1H-indole-3-carboxamide C099

Amount made: 9.5 mg. LCMS m/z 577 [M+H]⁺, purity (UV/MS) 100/90.

### N-(3,4-dichlorobenzyl)-7-isopropoxy-1-(3-(4-(3-phenoxypropyl)piperazin-1-yl)propyl)-1H-indole-3-carboxamide C100

Amount made: 4.0 mg. LCMS m/z 637 [M+H]⁺, purity (UV/MS) 84/40.

### N-(3,4-dichlorobenzyl)-1-(3-(dihydro-1H-pyrido[1,2-a]pyrazin-2(6H,7H,8H,9H,9aH)-yl)propyl)-7-methoxy-1H-indole-3-carboxamide C101

Amount made: 1.8 mg. LCMS m/z 529 [M+H]⁺, purity (UV/MS) 100/100.

### 1-(3-(1-benzylpyrrolidin-3-ylamino)propyl)-N-(3-fluorobenzyl)-7-methoxy-1H-indole-3-carboxamide C102

Amount made: 2.1 mg. LCMS m/z 515 [M+H]⁺, purity (UV/MS) 98/80.

### N-(3,4-dichlorobenzyl)-1-(3-(dihydro-1H-pyrido[1,2-a]pyrazin-2(6H,7H,8H,9H,9aH)-yl)propyl)-7-ethyl-1H-indole-3-carboxamide C103

Amount made: 3.3 mg. LCMS m/z 527 [M+H]⁺, purity (UV/MS) 99/100.

### 1-(3-(4-(2-(4-chlorophenoxy)ethyl)piperazin-1-yl)propyl)-N-(3-fluorobenzyl)-7-methoxy-1H-indole-3-carboxamide C104

Amount made: 8.1 mg. LCMS m/z 579 [M+H]⁺, purity (UV/MS) 100/100.

### 7-methoxy-N-(3-methylbenzyl)-1-(3-(4-phenethylpiperazin-1-yl)propyl)-1H-indole-3-carboxamide C105

Amount made: 7.4 mg. LCMS m/z 525 [M+H]⁺, purity (UV/MS) 100/90.

### 7-methoxy-N-(3-methylbenzyl)-1-(3-(4-(3-phenoxypropyl)piperazin-1-yl)propyl)-1H-indole-3-carboxamide C106

Amount made: 6.6 mg. LCMS m/z 555 [M+H]⁺, purity (UV/MS) 81/60.

### 1-(3-(4-(2-chloro-6-fluorobenzyl)-1,4-diazepan-1-yl)propyl)-7-methoxy-N-(3-methylbenzyl) 1H-indole-3-carboxamide C107

Amount made: 4.7 mg. LCMS m/z 577 [M+H]⁺, purity (UV/MS) 100/80.

### 7-methoxy-N-(3-methylbenzyl)-1-(3-(4-(2-morpholino-2-oxoethyl)piperazin-1-yl)propyl)-1H-indole-3-carboxamide C108

Amount made: 5.6 mg. LCMS m/z 548 [M+H]⁺, purity (UV/MS) 100/90.

### 1-(3-(4-(2-(diisopropylamino)ethyl)piperazin-1-yl)propyl)-7-methoxy-N-(3-methylbenzyl)-1H-indole-3-carboxamide C109

Amount made: 7.2 mg. LCMS m/z 548 [M+H]⁺, purity (UV/MS) 99/80.

### 1-(3-(1-benzylpyrrolidin-3-ylamino)propyl)-7-methoxy-N-(3-methylbenzyl)-1H-indole-3-carboxamide C110

Amount made: 3.8 mg. LCMS m/z 511 [M+H]⁺, purity (UV/MS) 99/70.

### N-(3,4-dichlorobenzyl)-1-(3-(dihydro-1H-pyrido[1,2-a]pyrazin-2(6H,7H,8H,9H,9aH)-yl)propyl)-7-isopropoxy-1H-indole-3-carboxamide C111

Amount made: 4.0 mg. LCMS m/z 557 [M+H]⁺, purity (UV/MS) 100/100.

### 1-(3-((1R,3r,5S)-3-(2-(3-chloro-4-fluorophenyl)-2-oxoethyl)-8-azabicyclo[3.2.1]octan-8-yl)propyl)-7-methoxy-N-(3-methylbenzyl)-1H-indole-3-carboxamide C112

Amount made: 7.6 mg. LCMS m/z 616 [M+H]⁺, purity (UV/MS) 95/70.

### N-(3,4-dichlorobenzyl)-7-methoxy-1-(3-(4-(3-phenoxypropyl)piperazin-1-yl)propyl)-1H-indole-3-carboxamide C113

Amount made: 6.9 mg. LCMS m/z 609 [M+H]⁺, purity (UV/MS) 98/40.

### N-(3-fluorobenzyl)-1-(3-((1S,4S)-5-(2-(4-fluorophenoxy)ethyl)-2,5-diazabicyclo[2.2.2]octan-2-yl)propyl)-7-methoxy-1H-indole-3-carboxamide C114

Amount made: 6.8 mg. LCMS m/z 589 [M+H]⁺, purity (UV/MS) 100/90.

### N-(3-fluorobenzyl)-1-(3-((1R,5S)-3-(2-(4-fluorophenoxy)ethyl)-3,8-diazabicyclo[3.2.1]octan-8-yl)propyl)-7-methoxy-1H-indole-3-carboxamide C115

Amount made: 3.9 mg. LCMS m/z 589 [M+H]⁺, purity (UV/MS) 99/70.

### N-(3,4-dichlorobenzyl)-1-(3-((1S,4S)-5-(2-(4-fluorophenoxy)ethyl)-2,5-diazabicyclo[2.2.2]octan-2-yl)propyl)-7-methoxy-1H-indole-3-carboxamide C116

Amount made: 1.7 mg. LCMS m/z 639 [M+H]⁺, purity (UV/MS) 100/90.

### N-(3,4-dichlorobenzyl)-1-(3-((1R,5S)-3-(2-(4-fluorophenoxy)ethyl)-3,8-diazabicyclo[3.2.1]octan-8-yl)propyl)-7-methoxy-1H-indole-3-carboxamide C117

Amount made: 5.2 mg. LCMS m/z 639 [M+H]⁺, purity (UV/MS) 93/80.

### N-(3,4-dichlorobenzyl)-7-methoxy-1-(3-(4-(3-phenoxypropyl)-1,4-diazepan-1-yl)propyl in H-indole-3-carboxamide C118

Amount made: 9.6 mg. LCMS m/z 623 [M+H]⁺, purity (UV/MS) 100/90.

### 1-(3-((1R,3r,5S)-3-(2-(4-chlorophenyl)-2-oxoethyl)-8-azabicyclo[3.2.1]octan-8-yl)propyl)-N-(3,4-dichlorobenzyl)-7-methoxy-1H-indole-3-carboxamide C119

Amount made: 1.7 mg. LCMS m/z 652 [M+H]⁺, purity (UV/MS) 91/60.

### N-(3,4-dichlorobenzyl)-7-methoxy-1-(3-((1R,3r,5S)-3-(2-oxo-2-(pyridin-2-yl)ethyl)-8-azabicyclo[3.2.1]octan-8-yl)propyl)-1H-indole-3-carboxamide C120

Amount made: 4.0 mg. LCMS m/z 619 [M+H]⁺, purity (UV/MS) 99/90.

### 1-(3-((1S,4S)-5-(2-(4-fluorophenoxy)ethyl)-2,5-diazabicyclo[2.2.2]octan-2-yl)propyl)-7-methoxy-N-(2-methylbenzyl)-1H-indole-3-carboxamide C121

Amount made: 2.8 mg. LCMS m/z 585 [M+H]⁺, purity (UV/MS) 100/90.

### N-(3,4-dichlorobenzyl)-1-(3-(4-(2-(4-fluorophenoxy)ethyl)-1,4-diazepan-1-yl)propyl)-7-methoxy-1H-indole-3-carboxamide C122

Amount made: 3.4 mg. LCMS m/z 627 [M+H]⁺, purity (UV/MS) 99/90.

### 1-(3-((1R,5S)-3-(2-(4-fluorophenoxy)ethyl)-3,8-diazabicyclo[3.2.1]octan-8-yl)propyl)-7-methoxy-N-(2-methylbenzyl)-1H-indole-3-carboxamide C123

Amount made: 5.1 mg. LCMS m/z 585 [M+H]⁺, purity (UV/MS) 100/70.

### N-(3-fluorobenzyl)-1-(3-(4-(2-(4-fluorophenoxy)ethyl)-1,4-diazepan-1-yl)propyl)-7-methoxy-1H-indole-3-carboxamide C124

Amount made: 1.8 mg. LCMS m/z 577 [M+H]⁺, purity (UV/MS) 100/90.

### N-(3-fluorobenzyl)-7-methoxy-1-(3-(4-(2-phenoxyethyl)piperidin-1-yl)propyl)-1H-indole-3-carboxamide C125

Amount made: 2.9 mg. LCMS m/z 544 [M+H]⁺, purity (UV/MS) 100/70.

### N-(3-fluorobenzyl)-7-methoxy)-1-(3-((1R,3r,5S)-3-(2-oxo-2-(pyridin-2-yl)ethyl)-8-azabicyclo[3.2.1]octan-8-yl)propyl)-1H-indole-3-carboxamide C126

Amount made: 1.2 mg. LCMS m/z 569 [M+H]⁺, purity (UV/MS) 92/70.

### 1-(3-((1R,3r,5S)-3-(2-(4-chlorophenyl)-2-oxoethyl)-8-azabicyclo[3.2.1]octan-8-yl)propyl)-N-(3-fluorobenzyl)-7-methoxy-1H-indole-3-carboxamide C127

Amount made: 5.3 mg. LCMS m/z 602 [M+H]⁺, purity (UV/MS) 100/70.

### 1-(3-((1R,3r,5S)-3-(2-(3,4-dichlorophenyl)-2-oxoethyl)-8-azabicyclo[3.2.1]octan-8-yl)propyl)-N-(3-fluorobenzyl)-7-methoxy-1H-indole-3-carboxamide C128

Amount made: 5.6 mg. LCMS m/z 636 [M+H]⁺, purity (UV/MS) 100/90.

### 1-(3-(4-(2-(4-fluorophenoxy)ethyl 1,4-diazepan-1-yl)propyl)-7-methoxy-N-(2-methylbenzyl)-1H-indole-3-carboxamide C129

Amount made: 3.1 mg. LCMS m/z 573 [M+H]⁺, purity (UV/MS) 100/90.

### N-(3-fluorobenzyl)-7-methoxy-1-(3-(4-(3-phenoxypropyl)-1,4-diazepan-1-yl)propyl)-1H-indole-3-carboxamide C130

Amount made: 2.4 mg. LCMS m/z 573 [M+H]⁺, purity (UV/MS) 100/90.

### N-(3,4-dichlorobenzyl)-7-methoxy-1-(3-(4-(2-phenoxyethyl)piperidin-1-yl)propyl)-1H-indole-3-carboxamide C131

Amount made: 3.2 mg. LCMS m/z 594 [M+H]⁺, purity (UV/MS) 90/70.

### N-(3,4-dichlorobenzyl)-7-ethyl-1-(3-((1R,3r,5S)-3-(2-oxo-2-(pyridin-2-yl)ethyl)-8-azabicyclo[3.2.1]octan-8-yl)propyl)-1H-indole-3-carboxamide C132

Amount made: 1.8 mg. LCMS m/z 617 [M+H]⁺, purity (UV/MS) 99/80.

### 7-methoxy-N-(2-methylbenzyl)-1-(3-(4-(2-phenoxyethyl)piperidin-1-yl)propyl)-1H-indole-3-carboxamide C133

Amount made: 4.5 mg. LCMS m/z 540 [M+H]⁺, purity (UV/MS) 99/60.

### 1-(3-(1-benzylpyrrolidin-3-ylamino)propyl)-7-methoxy-N-(2-methylbenzyl)-1H-indole-3-carboxamide C134

Amount made: 2.9 mg. LCMS m/z 511 [M+H]⁺, purity (UV/MS) 99/80.

### 1-(3-((1R,3r,5S)-3-(2-(3-chloro-4-fluorophenyl)-2-oxoethyl)-8-azabicyclo[3.2.1]octan-8-yl)propyl)-7-methoxy-N-(2-methylbenzyl)-1H-indole-3-carboxamide C135

Amount made: 1.5 mg. LCMS m/z 616 [M+H]⁺, purity (UV/MS) 83/50.

### 7-methoxy-N-(2-methylbenzyl)-1-(3-(4-(2-phenoxyethyl)-1,4-diazepan-1-yl)propyl)-1H-indole-3-carboxamide C136

Amount made: 2.2 mg. LCMS m/z 555 [M+H]⁺, purity (UV/MS) 98/80.

### 7-methoxy-N-(2-methylbenzyl)-1-(3-((1R,3r,5S)-3-(2-oxo-2-(pyridin-2-yl)ethyl)-8-azabicyclo[3.2.1]octan-8-yl)propyl)-1H-indole-3-carboxamide C137

Amount made: 3.8 mg. LCMS m/z 565 [M+H]⁺, purity (UV/MS) 96/80.

### 1-(3-(dihydro-1H-pyrido[1,2-a]pyrazin-2(6H,7H,8H,9H,9aH)-yl)propyl)-N-(3-fluorobenzyl)-7-methoxy-1H-indole-3-carboxamide C138

Amount made: 3.0 mg. LCMS m/z 479 [M+H]⁺, purity (UV/MS) 99/80.

### 1-(3-((1R,3r,5S)-3-(2-(4-chlorophenyl)-2-oxoethyl)-8-azabicyclo[3.2.1]octan-8-yl)propyl)-7-methoxy-N-(2-methylbenzyl)-1H-indole-3-carboxamide C139

Amount made: 1.2 mg. LCMS m/z 598 [M+H]⁺, purity (UV/MS) 98/80.

### 1-(3-((1R,3r,5S)-3-(2-(3,4-dichlorophenyl)-2-oxoethyl)-8-azabicyclo[3.2.1]octan-8-yl))propyl)-7-methoxy-N-(2-methylbenzyl)-1H-indole-3-carboxamide C140

Amount made: 2.7 mg. LCMS m/z 632 [M+H]⁺, purity (UV/MS) 100/80.

### 7-methoxy-N-(2-methylbenzyl)-1-(3-(4-(3-phenoxypropyl)-1,4-diazepan-1-yl)propyl)-1H-indole-3-carboxamide C141

Amount made: 5.0 mg. LCMS m/z 569 [M+H]⁺, purity (UV/MS) 100/90.

### 1-(3-(2,3-dihydro-1H-inden-2-ylamino)propyl)-7-methoxy-N-(3-methylbenzyl)-1H-indole-3-carboxamide C142

Amount made: 4.6 mg. LCMS m/z 468 [M+H]⁺, purity (UV/MS) 89/70.

### 1-(3-(2,3-dihydro-1H-inden-2-ylamino)propyl)-N-isobutyl-7-methoxy-1H-indole-3-carboxamide C143

Amount made: 5.2 mg. LCMS m/z 420 [M+H]⁺, purity (UV/MS) 97/80.

### 1-(3-(3-(2-(4-chlorophenyl)-2-oxoethyl)-8-azabicyclo[3.2.1]octan-8-yl)propyl)-7-methoxy-N-(3-methylbenzyl)-1H-indole-3-carboxamide C144

Amount made: 7.6 mg. LCMS m/z 598 [M+H]⁺, purity (UV/MS) 98/70.

### 1-(3-(3-(2-(4-chlorophenyl)-2-oxoethyl)-8-azabicyclo[3.2.1]octan-8-yl)propyl)-N-isobutyl-7-methoxy-1H-indole-3-carboxamide C145

Amount made: 6.4 mg. LCMS m/z 550 [M+H]⁺, purity (UV/MS) 98/70.

### 1-(3-(3-(2-chlorobenzyl)piperidin-1-yl)propyl)-7-methoxy-N-(3-methylbenzyl)-1H-indole-3-carboxamide C146

Amount made: 4.3 mg. LCMS m/z 544 [M+H]⁺, purity (UV/MS) 97/80.

### 1-(3-(3-(2-chlorophenoxy)-8-azabicyclo[3.2.1]octan-8-yl)propyl)-7-methoxy-N-(3-methylbenzyl)-1H-indole-3-carboxamide C147

Amount made: 5.4 mg. LCMS m/z 572 [M+H]⁺, purity (UV/MS) 100/80.

### 1-(3-(3-(2-chlorophenoxy)-8-azabicyclo[3.2.1]octan-8-yl)propyl)-N-isobutyl-7-methoxy-1H-indole-3-carboxamide C148

Amount made: 4.6 mg. LCMS m/z 524 [M+H]⁺, purity (UV/MS) 98/80.

### 1-(3-(3-(4-chlorophenethyl)-8-azabicyclo[3.2.1]octan-8-yl)propyl)-7-methoxy-N-(3-methylbenzyl)-1H-indole-3-carboxamide C149

Amount made: 6.9 mg. LCMS m/z 584 [M+H]⁺, purity (UV/MS) 94/60.

### 1-(3-(3-(4-chlorophenethyl)-8-azabicyclo[3.2.1]octan-8-yl)propyl)-N-isobutyl-7-methoxy-1H-indole-3-carboxamide C150

Amount made: 5.8 mg. LCMS m/z 536 [M+H]⁺, purity (UV/MS) 97/70.

### 1-(3-(3-(4-chlorophenoxy)-8-azabicyclo[3.2.1]octan-8-yl)propyl)-7-methoxy-N-(3-methylbenzyl)-1H-indole-3-carboxamide C151

Amount made: 4.4 mg. LCMS m/z 572 [M+H]⁺, purity (UV/MS) 87/70.

### 1-(3-(3-(4-chloro-henoxy)-8-azabicyclo[3.2.1]octan-8-yl)propyl)-N-isobutyl-7-methoxy-1H-indole-3-carboxamide C152

Amount made: 3.0 mg. LCMS m/z 524 [M+H]⁺, purity (UV/MS) 94/70.

### 1-(3-(3-(4-chlorophenoxy)piperidin-1-yl)propyl)-7-methoxy-N-(3-methylbenzyl)-1H-indole-3-carboxamide C153

Amount made: 4.3 mg. LCMS m/z 546 [M+H]⁺, purity (UV/MS) 100/90.

### 1-(3-(3-(4-chlorophenoxy)piperidin-1-yl)propyl)-N-isobutyl-7-methoxy-1H-indole-3-carboxamide C154

Amount made: 5.8 mg. LCMS m/z 498 [M+H]⁺, purity (UV/MS) 100/90.

### 1-(3-(3-(4-fluorophenoxy)-8-azabicyclo[3.2.1]octan-8-yl)propyl)-7-methoxy-N-(3-methylbenzyl)-1H-indole-3-carboxamide C155

Amount made: 5.6 mg. LCMS m/z 556 [M+H]⁺, purity (UV/MS) 92/60.

### 1-(3-(3-(4-fluorophenoxy)-8-azabicyclo[3.2.1]octan-8-yl)propyl)-N-isobutyl-7-methoxy-1H-indole-3-carboxamide C156

Amount made: 4.2 mg. LCMS m/z 508 [M+H]⁺, purity (UV/MS) 98/80.

### 1-(3-(4-(2-(4-chloronaphthalen-1-yloxy)ethyl)piperazin-1-yl)propyl)-7-methoxy-N-(2-methylbenzyl)-1H-indole-3-carboxamide C157

Amount made: 2.1 mg. LCMS m/z 625 [M+H]⁺, purity (UV/MS) 100/100.

### 1-(3-(4-(2-(4-chloronaphthalen-1-yloxy)ethyl)piperazin-1-yl)propyl)-7-methoxy-N-(3-methylbenzyl)-1H-indole-3-carboxamide C158

Amount made: 0.9 mg. LCMS m/z 625 [M+H]⁺, purity (UV/MS) 100/90.

### 1-(3-(4-(2-(4-chloronaphthalen-1-yloxy)ethyl)piperazin-1-yl)propyl)-N-isobutyl-7-methoxy-1H-indole-3-carboxamide C159

Amount made: 0.8 mg. LCMS m/z 577 [M+H]⁺, purity (UV/MS) 100/90,

### 1-(3-(4-(2-(4-chlorophenoxy)ethyl)piperazin-1-yl)propyl)-7-methoxy-N-(3-methylbenzyl)-1H-indole-3-carboxamide C160

Amount made: 6.6 mg. LCMS m/z 575 [M+H]⁺, purity (UV/MS) 100/90.

### 1-(3-(4-(2-(4-chlorophenoxy)ethyl)piperazin-1-yl)propyl)-N-(3,4-dichlorobenzyl)-7-ethyl-1H-indole-3-carboxamide C161

Amount made: 8.8 mg. LCMS m/z 627 [M+H]⁺, purity (UV/MS) 100/100.

### 1-(3-(4-(2-(4-chlorophenoxy)ethyl)piperazin-1-yl)propyl)-N-isobutyl-7-methoxy-1H-indole-3-carboxamide C162

Amount made: 7.8 mg. LCMS m/z 527 [M+H]⁺, purity (UV/MS) 100/90.

### 1-(3-(4-(2-(4-chlorophenoxy)ethyl)piperidin-1-yl)propyl)-7-methoxy-N-(3-methylbenzyl)-1H-indole-3-carboxamide C163

Amount made: 3.1 mg. LCMS m/z 574 [M+H]⁺, purity (UV/MS) 100/90.

### 1-(3-(4-(2-(4-chlorophenoxy)ethyl)piperidin-1-yl)propyl)-N-isobutyl-7-methoxy-1H-indole-3-carboxamide C164

Amount made: 0.6 mg. LCMS m/z 526 [M+H]⁺, purity (UV/MS) 100/90.

### 1-(3-(4-(2,4-dichlorobenzyl)piperazin-1-yl)propyl)-7-methoxy-N-(3-methylbenzyl)-1H-indole-3-carboxamide C165

Amount made: 2.4 mg. LCMS m/z 579 [M+H]⁺, purity (UV/MS) 100/90.

### 1-(3-(4-(2,4-dichlorobenzyl)piperazin-1-yl)propyl)-N-isobutyl-7-methoxy-1H-indole-3-carboxamide C166

Amount made: 7.3 mg. LCMS m/z 531 [M+H]⁺, purity (UV/MS) 100/100.

### 1-(3-(4-(2-chlorobenzyl)-1,4-diazepan-1-yl)propyl)-7-methoxy-N-(3-methylbenzyl)-1H-indole-3-carboxamide C167

Amount made: 4.3 mg. LCMS m/z 559 [M+H]⁺, purity (UV/MS) 97/80.

### 1-(3-(4-(2-chlorobenzyl)-1,4-diazepan-1-yl)propyl)N-isobutyl-7-methoxy-1H-indole-3-carboxamide C168

Amount made: 4.1 mg. LCMS m/z 511 [M+H]⁺, purity (UV/MS) 98/80.

### 1-(3-(4-(2-chlorophenoxy)piperidin-1-yl)propyl)-7-methoxy-N-(3-methylbenzyl)-1H-indole-3-carboxamide C169

Amount made: 9.8 mg. LCMS m/z 546 [M+H]⁺, purity (UV/MS) 89/50.

### 1-(3-(4-(2-chlorophenoxy)piperidin-1-yl)propyl)-N-isobutyl-7-methoxy-1H-indole-3-carboxamide C170

Amount made: 8.5 mg. LCMS m/z 498 [M+H]⁺, purity (UV/MS) 94/70.

### 1-(3-(4-(3-chloro-5-(trifluoromethyl)pyridin-2-yl)piperazin-1-yl)propyl)-7-methoxy-N-(3-methylbenzyl)-1H-indole-3-carboxamide C171

Amount made: 9.4 mg. LCMS m/z 600 [M+H]⁺, purity (UV/MS) 100/100.

### 1-(3-(4-(3-chloro-5-(trifluoromethyl)pyridin-2-yl)piperazin-1-yl)propyl)-N-(3-chlorobenzyl)-7-methoxy-1H-indole-3-carboxamide C172

Amount made: 2.4 mg. LCMS m/z 620 [M+H]⁺, purity (UV/MS) 100/100.

### 1-(3-(4-(3-chloro-5-(trifluoromethyl)pyridin-2-yl)piperazin-1-yl)propyl)N-isobutyl-7-methoxy-1H-indole-3-carboxamide C173

Amount made: 8.2 mg. LCMS m/z 552 [M+H]⁺, purity (UV/MS) 100/100.

### 1-(3-(4-(3-chlorophenoxy)piperidin-1-yl)propyl)-7-methoxy-N-(3-methylbenzyl)-1H-indole-3-carboxamide C174

Amount made: 4.9 mg. LCMS m/z 546 [M+H]⁺, purity (UV/MS) 100/80.

### 1-(3-(4-(3-chlorophenoxy)piperidin-1-yl)propyl)-N-isobutyl-7-methoxy-1H-indole-3-carboxamide C175

Amount made: 5.8 mg. LCMS m/z 498 [M+H]⁺, purity (UV/MS) 100/90.

### 1-(3-(4-(4-chlorophenoxy)piperidin-1-yl)propyl)-7-methoxy-N-(3-methylbenzyl)-1H-indole-3-carboxamide C176

Amount made: 6.6 mg. LCMS m/z 546 [M+H]⁺, purity (UV/MS) 78/50.

¹H NMR (400 MHz, CDCl₃) δ: 7.71-7.55 (m, 2H), 7.34-7.09 (m, 7H), 6.81-6.78 (m, 2H), 6.72-6.69 (m, 1H), 4.63-4.55 (m, 2H), 4.47-4.40 (m, 2H), 3.97 (s, 3H), 3.63-2.89 (m, 9H), 2.37 (s, 3H), 2.35-2.09 (m, 4H).

### 1-(3-(4-(4-chlorophenoxy)piperidin-1-yl)propyl)-N-isobutyl-7-methoxy-1H-indole-3-carboxamide C177

Amount made: 3.5 mg. LCMS m/z 498 [M+H]⁺, purity (UV/MS) 88/60.

### 1-(3-(4-(4-fluorobenzyl)-1,4-diazepan-1-yl)propyl)-7-methoxy-N-(3-methylbenzyl)-1H-indole-3-carboxamide C178

Amount made: 6.5 mg. LCMS m/z 543 [M+H]⁺, purity (UV/MS) 100/90.

### 1-(3-(4-(4-fluorobenzyl)-1,4-diazepan-1-yl)propyl)-N-isobutyl-7-methoxy-1H-indole-3-carboxamide C179

Amount made: 5.6 mg. LCMS m/z 495 [M+H]⁺, purity (UV/MS) 100/90.

### 1-(3-(4-(4-fluorophenoxy)piperidin-1-yl)propyl)-7-methoxy-N-(3-methylbenzyl)-1H-indole-3-carboxamide C180

Amount made: 2.5 mg. LCMS m/z 530 [M+H]⁺, purity (UV/MS) 94/70.

### 1-(3-(4-(4-fluorophenoxy)piperidin-1-yl)propyl)-N-isobutyl-7-methoxy-1H-indole-3-carboxamide C181

Amount made: 2.3 mg. LCMS m/z 482 [M+H]⁺, purity (UV/MS) 96/80.

### 1-(3-(4-(benzo[d]thiazol-2-yl)piperidin-1-yl)propyl)-7-methoxy-N-(3-methylbenzyl)-1H-indole-3-carboxamide C182

Amount made: 6.3 mg. LCMS m/z 553 [M+H]⁺, purity (UV/MS) 100/80.

### 1-(3-(4-(benzo[d]thiazol-2-yl)piperidin-1-yl)propyl)-N-(3-chlorobenzyl)-7-methoxy-1H-indole-3-carboxamide C183

Amount made: 3.3 mg. LCMS m/z 573 [M+H]⁺, purity (UV/MS) 98/70.

### 1-(3-(4-(benzo[d]thiazol-2-yl)piperidin-1-yl)propyl)-N-isobutyl-7-methoxy-1H-indole-3-carboxamide C184

Amount made: 6.9 mg. LCMS m/z 505 [M+H]⁺, purity (UV/MS) 97/70.

### 1-(3-(4-benzoylpiperidin-1-yl)propyl)-7-methoxy-N-(3-methylbenzyl)-1H-indole-3-carboxamide C185

Amount made: 3.8 mg. LCMS m/z 524 [M+H]⁺, purity (UV/MS) 95/60.

### 1-(3-(4-benzoylpiperidin-1-yl)propyl)-N-(3-chlorobenzyl)-7-methoxy-1H-indole-3-carboxamide C186

Amount made: 11.1 mg. LCMS m/z 544 [M+H]⁺, purity (UV/MS) 98/70.

### 1-(3-(4-benzoylpiperidin-1-yl)propyl)-N-isobutyl-7-methoxy-1H-indole-3-carboxamide C187

Amount made: 5.4 mg. LCMS m/z 476 [M+H]⁺, purity (UV/MS) 100/80.

### 1-(3-(4-butylpiperidin-1-yl)propyl)-7-methoxy-N-(3-methylbenzyl)-1H-indole-3-carboxamide C188

Amount made: 5.3 mg. LCMS m/z 476 [M+H]⁺, purity (UV/MS) 99/90.

### 1-(3-(4-butylpiperidin-1-yl)propyl)-N-(3-chlorobenzyl)-7-methoxy-1H-indole-3-carboxamide C189

Amount made: 5.8 mg. LCMS m/z 496 [M+H]⁺, purity (UV/MS) 99/90.

### 1-(3-(4-butylpiperidin-1-yl)propyl)-N-isobutyl-7-methoxy-1H-indole-3-carboxamide C190

Amount made: 2.8 mg. LCMS m/z 428 [M+H]⁺, purity (UV/MS) 100/90.

### 7-methoxy-1-(3-(4-(2-methoxyphenyl)piperidin-1-yl)propyl)-N-(3-methylbenzyl)-1H-indole-3-carboxamide C191

Amount made: 9.7 mg. LCMS m/z 526 [M+H]⁺, purity (UV/MS) 96/70.

### 7-methoxy-N-(3-methylbenzyl)-1-(3-(2-phenoxyethylamino)propyl)-1H-indole-3-carboxamide C192

Amount made: 4.8 mg. LCMS m/z 472 [M+H]⁺, purity (UV/MS) 91/70.

### 7-methoxy-N-(3-methylbenzyl)-1-(3-(3-(2-oxo-2-phenylethyl)-8-azabicyclo[3.2.1]octan-8-yl)propyl)-1H-indole-3-carboxamide C193

Amount made: 4.2 mg. LCMS m/z 564 [M+H]⁺, purity (UV/MS) 96/80.

### 7-methoxy-N-(3-methylbenzyl)-1-(3-(3-pentyl-8-azabicyclo[3.2.1]octan-8-yl)propyl)-1H-indole-3-carboxamide C194

Amount made: 2.8 mg. LCMS m/z 516 [M+H]⁺, purity (UV/MS) 97/90.

### 7-methoxy-N-(3-methylbenzyl)-1-(3-(3-phenethyl-8-azabicyclo[3.2.1]octan-8-yl)propyl)-1H-indole-3-carboxamide C195

Amount made: 5.0 mg. LCMS m/z 550 [M+H]⁺, purity (UV/MS) 91/70.

### 7-methoxy-N-(3-methylbenzyl)-1-(3-(4-(2-oxoindolin-1-yl)piperidin-1-yl)propyl)-1H-indole-3-carboxamide C196

Amount made: 0.9 mg. LCMS m/z 551 [M+H]⁺, purity (UV/MS) 94/80.

### 7-methoxy-N-(3-methylbenzyl)-1-(3-(4-(2-phenoxyethyl)piperazin-1-yl)propyl)-1H-indole-3-carboxamide C197

Amount made: 8.1 mg. LCMS m/z 541 [M+H]⁺, purity (UV/MS) 100/100.

### 7-methoxy-N-(3-methylbenzyl)-1-(3-(4-(3-(pyridin-3-yl)-1,2,4-oxadiazol-5-yl)piperidin-1-yl)propyl)-1H-indole-3-carboxamide C198

Amount made: 6.9 mg. LCMS m/z 565 [M+H]⁺, purity (UV/MS) 98/70.

### 7-methoxy-N-(3-methylbenzyl)-1-(3-(4-(3-(pyridin-4-yl)-1,2,4-oxadiazol-5-yl)piperidin-1-yl)propyl)-1H-indole-3-carboxamide C199

Amount made: 8.0 mg. LCMS m/z 565 [M+H]⁺, purity (UV/MS) 98/60.

### 7-methoxy-N-(3-methylbenzyl)-1-(3-(4-oxospiro[chroman-2,4′-piperidine]-1′-yl)propyl)-1H-indole-3-carboxamide C200

Amount made: 6.2 mg. LCMS m/z 552 [M+H]⁺, purity (UV/MS) 99/90.

### 7-methoxy-N-(3-methylbenzyl)-1-(3-(4-phenethyl-1,4-diazepan-1-yl)propyl)-1H-indole-3-carboxamide C201

Amount made: 6.3 mg. LCMS m/z 539 [M+H]⁺, purity (UV/MS) 100/90.

### N-(3,4-dichlorobenzyl)-7-isopropoxy-1-(3-((1R,3r,5S)-3-(2-oxo-2-(pyridin-2-yl)ethyl)-8-azabicyclo[3.2.1]octan-8-yl)propyl)-1H-indole-3-carboxamide C202

Amount made: 4.3 mg. LCMS m/z 647 [M+H]⁺, purity (UV/MS) 98/80.

### N-(3-chlorobenzyl)-1-(3-(2,3-dihydro-1H-inden-2-ylamino)propyl)-7-methoxy-1H-indole-3-carboxamide C203

Amount made: 5.7 mg. LCMS m/z 488 [M+H]⁺, purity (UV/MS) 94/90.

### N-(3-chlorobenzyl)-1-(3-(3-(2-(4-chlorophenyl)-2-oxoethyl)-8-azabicyclo[3.2.1]octan-8-yl)propyl)-7-methoxy-1H-indole-3-carboxamide C204

Amount made: 7.7 mg. LCMS m/z 618 [M+H]⁺, purity (UV/MS) 98/60.

### N-(3-chlorobenzyl)-1-(3-(3-(2-chlorobenzyl)piperidin-1-yl)propyl)-7-methoxy-1H-indole-3-carboxamide C205

Amount made: 4.2 mg. LCMS m/z 564 [M+H]⁺, purity (UV/MS) 98/80.

### N-(3-chlorobenzyl)-1-(3-(3-(2-chlorophenoxy)-8-azabicyclo[3.2.1]octan-8-yl)propyl)-7-methoxy-1H-indole-3-carboxamide C206

Amount made: 4.5 mg. LCMS m/z 592 [M+H]⁺, purity (UV/MS) 100/80.

### N-(3-chlorobenzyl)-1-(3-(3-(4-chlorophenethyl)-8-azabicyclo[3.2.1]octan-8-yl)propyl)-7-methoxy-1H-indole-3-carboxamide C207

Amount made: 6.4 mg. LCMS m/z 604 [M+H]⁺, purity (UV/MS) 97/60.

### N-(3-chlorobenzyl)-1-(3-(3-(4-chlorophenoxy)-8-azabicyclo[3.2.1]octan-8-yl)propyl)-7-methoxy-1H-indole-3-carboxamide C208

Amount made: 3.4 mg. LCMS m/z 592 [M+H]⁺, purity (UV/MS) 87/70.

### N-(3-chlorobenzyl)-1-(3-(3-(4-chlorophenoxy)piperidin-1-yl)propyl)-7-methoxy-1H-indole-3-carboxamide C209

Amount made: 7.2 mg. LCMS m/z 566 [M+H]⁺, purity (UV/MS) 100/90.

### N-(3-chlorobenzyl)-1-(3-(3-(4-fluorophenoxy)-8-azabicyclo[3.2.1]octan-8-yl)propyl)-7-methoxy-1H-indole-3-carboxamide C210

Amount made: 4.5 mg. LCMS m/z 576 [M+H]⁺, purity (UV/MS) 97/70.

### N-(3-chlorobenzyl)-1-(3-(4-(2-(4-chloronaphthalen-1-yloxy)ethyl)piperazin-1-yl)propyl)-7-methoxy-1H-indole-3-carboxamide C211

Amount made: 1.9 mg. LCMS m/z 645 [M+H]⁺, purity (UV/MS) 100/100.

### N-(3-chlorobenzyl)-1-(3-(4-(2-(4-chlorophenoxy)ethyl)piperazin-1-yl)propyl)-7-methoxy-1H-indole-3-carboxamide C212

Amount made-6.7 mg. LCMS m/z 595 [M+H]⁺, purity (UV/MS) 100/100.

### N-(3-chlorobenzyl)-1-(3-(4-(2-(4-chlorophenoxy)ethyl)piperidin-1-yl)propyl)-7-methoxy-1H-indole-3-carboxamide C213

Amount made: 4.2 mg. LCMS m/z 594 [M+H]⁺, purity (UV/MS) 100/90.

### N-(3-chlorobenzyl)-1-(3-(4-(2-chlorobenzyl)-1,4-diazepan-1-yl)propyl)-7-methoxy-1H-indole-3-carboxamide C214

Amount made: 4.0 mg. LCMS m/z 579 [M+H]⁺, purity (UV/MS) 100/90.

### N-(3-chlorobenzyl)-1-(3-(4-(2-chlorophenoxy)piperidin-1-yl)propyl)-7-methoxy-1H-indole-3-carboxamide C215

Amount made-5.5 mg. LCMS m/z 566 [M+H]⁺, purity (UV/MS) 93/70.

### N-(3-chlorobenzyl)-1-(3-(4-(3-chlorophenoxy)piperidin-1-yl)propyl)-7-methoxy-1H-indole-3-carboxamide C216

Amount made: 5.5 mg. LCMS m/z 566 [M+H]⁺, purity (UV/MS) 98/90.

### N-(3-chlorobenzyl)-1-(3-(4-(4-chlorophenoxy)piperidin-1-yl)propyl)-7-methoxy-1H-indole-3-carboxamide C217

Amount made: 4.2 mg. LCMS m/z 566 [M+H]⁺, purity (UV/MS) 79/60.

¹H NMR (400 MHz, CDCl₃) δ: 7.71-7.61 (m, 2H), 7.39-7.13 (m, 7H), 6.80-6.67 (m, 3H), 4.73-4.48 (m, 4H), 3.98 (s, 3H), 3.57-3.39 (m, 2H), 3.01-2.93 (m, 3H), 2.72-2.04 (m, 8H).

### N-(3-chlorobenzyl)-1-(3-(4-(4-fluorobenzyl)-1,4-diazepan-1-yl)propyl)-7-methoxy-1H-indole-3-carboxamide C218

Amount made: 5.9 mg. LCMS m/z 563 [M+H]⁺, purity (UV/MS) 100/90.

### N-(3-chlorobenzyl)-1-(3-(4-(4-fluorophenoxy)piperidin-1-yl)propyl)-7-methoxy-1H-indole-3-carboxamide C219

Amount made: 3.1 mg. LCMS m/z 550 [M+H]⁺, purity (UV/MS) 95/70.

### N-(3-chlorobenzyl)-7-methoxy-1-(3-(2-phenoxyethylamino)propyl)-1H-indole-3-carboxamide C220

Amount made: 3.7 mg. LCMS m/z 492 [M+H]⁺, purity (UV/MS) 96/70.

### N-(3-chlorobenzyl)-7-methoxy-1-(3-(3-(2-oxo-2-phenylethyl)-8-azabicyclo[3.2.1]octan-8-yl)propyl)-1H-indole-3-carboxamide C221

Amount made: 3.5 mg. LCMS m/z 584 [M+H]⁺, purity (UV/MS) 95/80.

### N-(3-chlorobenzyl)-7-methoxy-1-(3-(3-pentyl-8-azabicyclo[3.2.1]octan-8-yl)propyl)-1H-indole-3-carboxamide C222

Amount made: 3.5 mg. LCMS m/z 536 [M+H]⁺, purity (UV/MS) 95/90.

### N-(3-chlorobenzyl)-7-methoxy-1-(3-(3-phenethyl-8-azabicyclo[3.2.1]octan-8-yl)propyl)-1H-indole-3-carboxamide C223

Amount made: 5.5 mg. LCMS m/z 570 [M+H]⁺, purity (UV/MS) 93/80.

### N-(3-chlorobenzyl)-7-methoxy-1-(3-(4-(2-methoxyphenyl)piperidin-1-yl)propyl)-1H-indole-3-carboxamide C224

Amount made: 4.0 mg. LCMS m/z 546 [M+H]⁺, purity (UV/MS) 96/80.

### N-(3-chlorobenzyl)-7-methoxy-1-(3-(4-(2-oxoindolin-1-yl)piperidin-1-yl)propyl)-1H-indole-3-carboxamide C225

Amount made: 1.1 mg. LCMS m/z 571 [M+H]⁺, purity (UV/MS) 97/90.

### N-(3-chlorobenzyl)-7-methoxy-1-(3-(4-(2-phenoxyethyl)piperazin-1-yl)propyl)-1H-indole-3-carboxamide C226

Amount made: 7.6 mg. LCMS m/z 561 [M+H]⁺, purity (UV/MS) 100/100.

### N-(3-chlorobenzyl)-7-methoxy-1-(3-(4-(3-(pyridin-3-yl)-1,2,4-oxadiazol-5-yl)piperidin-1-yl)propyl)-1H-indole-3-carboxamide C227

Amount made: 5.6 mg. LCMS m/z 585 [M+]⁺, purity (UV/MS) 98/60.

### N-(3-chlorobenzyl)-7-methoxy-1-(3-(4-(3-(pyridin-4-yl)-1,24-oxadiazol-5-yl)piperidin-1-yl)propyl)-1H-indole-3-carboxamide C228

Amount made: 6.8 mg. LCMS m/z 585 [M+H]⁺, purity (UV/MS) 98/60.

### N-(3-chlorobenzyl)-7-methoxy-1-(3-(4-oxospiro[chroman-2,4′-piperidine]-1′-yl)propyl)-1H-indole-3-carboxamide C229

Amount made: 6.1 mg. LCMS m/z 572 [M+H]⁺, purity (UV/MS) 100/90.

### N-(3-chlorobenzyl)-7-methoxy-1-(3-(4-phenethyl-1,4-diazepan-1-yl)propyl)-1H-indole-3-carboxamide C230

Amount made: 6.3 mg. LCMS m/z 559 [M+H]⁺, purity (UV/MS) 100/90.

### N-benzyl-1-(3-((1R,3r,5S)-3-(2-(3,4-dichlorophenyl)-2-oxoethyl)-g-azabicyclo[3.2.1]octan-8-yl)propyl)-7-ethyl-1H-indole-3-carboxamide C231

Amount made: 4.8 mg. LCMS m/z 616 [M+H]⁺, purity (UV/MS) 93/40.

### N-benzyl-1-(3-((1R,3r,5S)-3-(2-(4-chlorophenyl)-2-oxoethyl)-8-azabicyclo[3.2.1]octan-8-yl)propyl)-7-ethyl-1H-indole-3-carboxamide C232

Amount made: 4.5 mg. LCMS m/z 582 [M+H]⁺, purity (UV/MS) 99/80.

### N-benzyl-1-(3-(4-(2-(4-chloronaphthalen-1-yloxy)ethyl)piperazin-1-yl)propyl)-7-ethyl-1H-indole-3-carboxamide C233

Amount made: 4.4 mg. LCMS m/z 609 [M+H]⁺, purity (UV/MS) 100/90.

### N-benzyl-7-ethyl-1-(3-((1R,3r, 5S)-3-(2-oxo-2-(pyridin-2-yl)ethyl)-8-azabicyclo[3.2.1]octan-8-yl)propyl)-1H-indole-3-carboxamide C234

Amount made: 2.4 mg. LCMS m/z 549 [M+H]⁺, purity (UV/MS) 100/100.

### N-benzyl-7-ethyl-1-(3-(4-C2-morpholino-2-oxoethyl)piperazin-1-yl)propyl)-1H-indole-3-carboxamide C235

Amount made: 9.8 mg. LCMS m/z 532 [M+H]⁺, purity (UV/MS) 100/80.

### N-benzyl-7-ethyl-1-(3-(4-(2-phenoxyethyl)piperazin-1-yl)propyl)-1H-indole-3-carboxamide C236

Amount made: 6.1 mg. LCMS m/z 525 [M+H]⁺, purity (UV/MS) 100/90.

### N-benzyl-7-ethyl-1-(3-(4-(3-phenoxypropyl)-1,4-diazepan-1-yl)propyl)-1H-indole-3-carboxamide C237

Amount made: 3.0 mg. LCMS m/z 553 [M+H]⁺, purity (UV/MS) 100/100.

### N-benzyl-7-ethyl-1-(3-(4-phenethyl-1,4-diazepan-1-yl)propyl)-1H-indole-3-carboxamide C238

Amount made: 5.3 mg. LCMS m/z 523 [M+H]⁺, purity (UV/MS) 98/80.

### N-isobutyl-7-methoxy-1-(3-(2-phenoxyethylamino)propyl)-1H-indole-3-carboxamide C239

Amount made: 4.7 mg. LCMS m/z 424 [M+H]⁺, purity (UV/MS) 95/80.

### N-isobutyl-7-methoxy-1-(3-(3-(2-oxo-2-phenylethyl)-8-azabicyclo[3.2.1]octan-8-yl)propyl)-1H-indole-3-carboxamide C240

Amount made: 4.0 mg. LCMS m/z 516 [M+H]⁺, purity (UV/MS) 98/80.

### N-isobutyl-7-methoxy-1-(3-(3-pentyl-8-azabicyclo[3.2.1]octan-8-yl)propyl)-1H-indole-3-carboxamide C241

Amount made: 2.1 mg. LCMS m/z 468 [M+H]⁺, purity (UV/MS) 87/80.

### N-isobutyl-7-methoxy-1-(3-(3-phenethyl-8-azabicyclo[3.2.1]octan-8-yl)propyl)-1H-indole-3-carboxamide C242

Amount made: 4.1 mg. LCMS m/z 502 [M+H]⁺, purity (UV/MS) 90/70.

### N-isobutyl-7-methoxy-1-(3-(4-(2-methoxyphenyl)piperidin-1-yl)propyl)-1H-indole-3-carboxamide C243

Amount made: 2.2 mg. LCMS m/z 478 [M+H]⁺, purity (UV/MS) 96/70.

### N-isobutyl-7-methoxy-1-(3-(4-(2-phenoxyethyl)piperazin-1-yl)propyl)-1H-indole-3-carboxamide C244

Amount made: 9.0 mg. LCMS m/z 493 [M+H]⁺, purity (UV/MS) 94/70.

### N-isobutyl-7-methoxy-1-(3-(4-(3-(pyridin-3-yl)-1,2,4-oxadiazol-5-yl)piperidin-1-yl)propyl)-1H-indole-3-carboxamide C245

Amount made: 6.0 mg. LCMS m/z 517 [M+H]⁺, purity (UV/MS) 99/70.

### N-isobutyl-7-methoxy-1-(3-(4-(3-(pyridin-4-yl)-1,2,4-oxadiazol-5-yl)piperidin-1-yl)propyl)-1H-indole-3-carboxamide C246

Amount made: 6.9 mg. LCMS m/z 517 [M+H]⁺, purity (UV/MS) 97/60.

### N-isobutyl-7-methoxy-1-(3-(4-oxospiro[chroman-2,4′-piperidine]-1′-yl)propyl)-1H-indole-3-carboxamide C247

Amount made: 6.3 mg. LCMS m/z 504 [M+H]⁺, purity (UV/MS) 100/90.

### N-isobutyl-7-methoxy-1-(3-(4-phenethyl-1,4-diazepan-1-yl)propyl)-1H-indole-3-carboxamide C248

Amount made: 6.8 mg. LCMS m/z 491 [M+H]⁺, purity (UV/MS) 100/90.

### 1-(3-(4-(3-cyanopyridin-2-yl)-1,4-diazepan-1-yl)propyl)-N-(2-ethylhexyl)-1H-indole-3-carboxamide C249

Amount made: 9.3 mg. LCMS m/z 515 [M+H]⁺, purity (UV/MS) 97/75.

### 1-(3-(4-(3-cyanopyridin-2-yl)-1,4-diazepan-1-yl)propyl)-N-(3-methylbenzyl)-1H-indole-3-carboxamide C250

Amount made: 7.5 mg. LCMS m/z 507 [M+H]⁺, purity (UV/MS) 95/66.

### 1-(3-(4-(3-cyanopyridin-2-yl)-1,4-diazepan-1-yl)propyl)-N-isobutyl-1H-indole-3-carboxamide C251

Amount made: 5.1 mg. LCMS m/z 459 [M+H]⁺, purity (UV/MS) 96/77.

### 1-(3-(4-(4-chlorophenylthio)piperidin-1-yl)propyl)-N-(2-ethylhexyl)-1H-indole-3-carboxamide hydrochloride C252

Amount made: 4.3 mg. LCMS m/z 540 [M+H]⁺, purity (UV/MS) 90/60.

### 1-(3-(4-(4-chlorophenylthio)piperidin-1-yl)propyl)-N-(3-methylbenzyl)-1H-indole-3-carboxamide C253

Amount made: 10.2 mg. LCMS m/z 532 [M+H]⁺, purity (UV/MS) 100/63.

### 1-(3-(4-(4-chlorophenylthio)piperidin-1-yl)propyl)-N-isobutyl-1H-indole-3-carboxamide hydrochloride C254

Amount made: 1.3 mg. LCMS m/z 484 [M+H]⁺, purity (UV/MS) 75/54.

### 1-(3-(4-(4-fluorophenoxy)piperidin-1-yl)propyl)-N-(3-methylbenzyl)-1H-indole-3-carboxamide C255

Amount made: 7.9 mg. LCMS m/z 500 [M+H]⁺, purity (UV/MS) 95/100.

### 1-(3-(4-(4-fluorophenoxy)piperidin-1-yl)propyl)-N-isobutyl-1H-indole-3-carboxamide hydrochloride C256

Amount made: 3.9 mg. LCMS m/z 452 [M+H]⁺, purity (UV/MS) 81/58.

### 1-(3-(4-(benzo[d]thiazol-2-yl)piperidin-1-yl)propyl)-N-(2-chlorobenzyl-1H-indole-3-carboxamide C257

Amount made: 5.3 mg, LCMS m/z 543 [M+H]⁺, purity (UV/MS) 96/66.

### 1-(3-(4-(benzo[d]thiazol-2-yl)piperidin-1-yl)propyl)-N-(2-ethylhexyl)-1H-indole-3-carboxamide C258

Amount made: 7.2 mg. LCMS m/z 531 [M+H]⁺, purity (UV/MS) 91/62.

### 1-(3-(4-(benzo[d]thiazol-2-ylpiperidin-1-yl)propyl)-N-(3-chlorobenzyl)-1H-indole-3-carboxamide C259

Amount made: 5.0 mg. LCMS m/z 543 [M+H]⁺, purity (UV/MS) 100/93.

### 1-(3-(4-(benzo[d]thiazol-2-yl)piperidin-1-yl)propyl)-N-(3-methylbenzyl)-1H-indole-3-carboxamide C260

Amount made: 4.9 mg. LCMS m/z 523 [M+H]⁺, purity (UV/MS) 91/66.

### 1-(3-(4-(benzo[d]thiazol-2-yl)piperidin-1-yl)propyl)-N-(4-chlorobenzyl)-1H-indole-3-carboxamide C261

Amount made: 5.0 mg. LCMS m/z 543 [M+H]⁺, purity (UV/MS) 87/78.

### 1-(3-(4-(benzo[d]thiazol-2-yl)piperidin-1-yl)propyl)-N-isobutyl-1H-indole-3-carboxamide C262

Amount made: 4.8 mg. LCMS m/z 475 [M+H]⁺, purity (UV/MS) 97/80.

### 1-(3-(4-(benzylpiperidin-1-yl)propyl)-N-(2-chlorobenzyl)-1H-indole-3-carboxamide C263

Amount made: 8.7 mg. LCMS m/z 500 [M+H]⁺, purity (UV/MS) 96/63.

### 1-(3-(4-(benzylpiperidin-1-yl)propyl)-N-(2-ethylhexyl)-1H-indole-3-carboxamide C264

Amount made: 8.3 mg. LCMS m/z 488 [M+H]⁺, purity (UV/MS) 92/70.

### 1-(3-(4-(benzylpiperidin-yl)propyl)-N-(3-chlorobenzyl)-1H-indole-3-carboxamide C265

Amount made: 7.2 mg. LCMS m/z 500 [M+H]⁺, purity (UV/MS) 100/80.

### 1-(3-(4-(benzylpiperidin-1-yl)propyl)-N-(3-methylbenzyl)-1H-indole-3-carboxamide C266

Amount made: 6.1 mg. LCMS m/z 480 [M+H]⁺, purity (UV/MS) 100/94.

### 1-(3-(4-(benzylpiperidin-1-yl)propyl)-N-(4-chlorobenzyl)-1H-indole-3-carboxamide C267

Amount made: 8.2 mg. LCMS m/z 500 [M+H]⁺, purity (UV/MS) 98/67.

### 1-(3-(4-(benzylpiperidin-1-yl)propyl)-N-isobutyl-1H-indole-3-carboxamide C268

Amount made: 5.3 mg. LCMS m/z 432 [M+H]⁺, purity (UV/MS) 95/89.

### 1-(3-(4-butylpiperidin-1-yl)propyl)-N-(2-chlorobenzyl)-1H-indole-3-carboxamide C269

Amount made: 8.5 mg. LCMS m/z 466 [M+H]⁺, purity (UV/MS) 66/57.

### 1-(3-(4-butylpiperidin-1-yl)propyl)-N-(2-ethylhexyl)-1H-indole-3-carboxamide C270

Amount made: 11.1 mg. LCMS m/z 454 [M+H]⁺, purity (UV/MS) 97/85.

### 1-(3-(4-butylpiperidin-1-yl)propyl)-N-(3-chlorobenzyl)-1H-indole-3-carboxamide C271

Amount made: 6.1 mg. LCMS m/z 466 [M+H]⁺, purity (UV/MS) 99/80.

### 1-(3-(4-butylpiperidin-1-yl)propyl)-N-(3-methylbenzyl)-1H-indole-3-carboxamide C272

Amount made: 10.2 mg. LCMS m/z 446 [M+H]⁺, purity (UV/MS) 100/89.

### 1-(3-(4-butylpiperidin-1-yl)propyl)-N-(4-chlorobenzyl)-1H-indole-3-carboxamide C273

Amount made: 6.1 mg. LCMS m/z 466 [M+H]⁺, purity (UV/MS) 98/44.

### 1-(3-(4-butylpiperidin-1-yl)propyl)-N-isobutyl-1H-indole-3-carboxamide C274

Amount made: 5.4 mg. LCMS m/z 398 [M+H]⁺, purity (UV/MS) 100/83.

### 1-(3-(methyl(2-(pyridin-2-yl)ethyl)amino)propyl)-N-(3-methylbenzyl-1H-indole-3-carboxamide C275

Amount made: 9.3 mg. LCMS m/z 441 [M+H]⁺, purity (UV/MS) 95/82.

### N-(2-chlorobenzyl)-1-(3-(2-phenyl)propylamino)propyl)-1H-indole-3-carboxamide C276

Amount made: 4.2 mg. LCMS m/z 460 [M+H]⁺, purity (UV/MS) 98/94.

### N-(2-chlorobenzyl)-1-(3-(3-phenethyl-8-azabicyclo[3.2.1]octan-8-yl)propyl)-1H-indole-3-carboxamide C277

Amount made: 8.6 mg. LCMS m/z 540 [M+H]⁺, purity (UV/MS) 85/30.

### N-(2-chlorobenzyl)-1-(3-(4-(3-cyanopyridin-2-yl)-1,4-diazepan-1-yl)propyl)-1H-indole-3-carboxamide C278

Amount made: 9.1 mg. LCMS m/z 527 [M+H]⁺, purity (UV/MS) 96/63.

### N-(2-chlorobenzyl)-1-(3-(4-(4-chlorophenylthio)piperidin-1-yl)propyl)-1H-indole-3-carboxamide C279

Amount made: 6.9 mg. LCMS m/z 552 [M+H]⁺, purity (UV/MS) 87/81.

### N-(2-chlorobenzyl)-1-(3-(4-(4-fluorophenoxy)piperidin-1-yl)propyl)-1H-indole-3-carboxamide C280

Amount made: 9.2 mg. LCMS m/z 520 [M+H]⁺, purity (UV/MS) 94/69.

### N-(2-chlorobenzyl)-1-(3 (4-(pyrrolidin-1-yl)piperidin-1-yl)propyl)-1H-indole-3-carboxamide C281

Amount made: 4.4 mg. LCMS m/z 479 [M+H]⁺, purity (UV/MS) 70/93.

### N-(2-chlorobenzyl)-1-(3-(methyl(2-(pyridin-2-yl)ethyl)amino)propyl)-1H-indole-3-carboxamide C282

Amount made: 15.0 mg. LCMS m/z 461 [M+H]⁺, purity (UV/MS) 86/82.

### N-(2-ethylhexyl)-1-(3-(2-phenyl)propylamino)propyl)-1H-indole-3-carboxamide C283

Amount made: 9.8 mg. LCMS m/z 448 [M+H]⁺, purity (UV/MS) 98/80.

### N-(2-ethylhexyl)-1-(3-(3-phenethyl-8-azabicyclo[3.2.1]octan-8-yl)propyl)-1H-indole-3-carboxamide C284

Amount made: 4.3 mg. LCMS m/z 528 [M+H]⁺, purity (UV/MS) 98/41.

### N-(2-ethylhexyl)-1-(3-(4-(4-fluorophenoxy)piperidin-1-yl)propyl)-1H-indole-3-carboxamide hydrochloride C285

Amount made: 5.8 mg. LCMS m/z 508 [M+H]⁺, purity (UV/MS) 86/59.

### N-(2-ethylhexyl)-1-(3-(4-(pyrrolidin-1-yl)piperidin-1-yl)propyl)-1H-indole-3-carboxamide C286

Amount made: 8.3 mg. LCMS m/z 467 [M+H]⁺, purity (UV/MS) 87/27.

### N-(2-ethylhexyl)-1-(3-(methyl(2-(pyridin-2-yl)ethyl)amino)propyl)-1H-indole-3-carboxamide C287

Amount made: 8.5 mg. LCMS m/z 449 [M+H]⁺, purity (UV/MS) 93/80.

### N-(3-chlorobenzyl)-1-(3-(2-phenyl)propylamino)propyl)-1H-indole-3-carboxamide C288

Amount made: 1.5 mg. LCMS m/z 460 [M+H]⁺, purity (UV/MS) 100/96.

### N-(3-chlorobenzyl)-1-(3-(3-phenethyl-8-azabicyclo[3.2.1]octan-8-yl)propyl)-1H-indole-3-carboxamide C289

Amount made: 4.3 mg. LCMS m/z 540 [M+H]⁺, purity (UV/MS) 94/82.

### N-(3-chlorobenzyl)-1-(3-(4-(3-cyanopyridin-2-yl)-1,4-diazepan-1-yl)propyl)-1H-indole-3-carboxamide C290

Amount made: 5.5 mg. LCMS m/z 527 [M+H]⁺, purity (UV/MS) 99/74.

### N-(3-chlorobenzyl)-1-(3-(4-(4-chlorophenylthio)piperidin-1-yl)propyl)-1H-indole-3-carboxamide C291

Amount made: 2.6 mg. LCMS m/z 552 [M+H]⁺, purity (UV/MS) 91/72.

### N-(3-chlorobenzyl)-1-(3-(4-(4-fluorophenoxy)piperidin-1-yl)propyl)-1H-indole-3-carboxamide C292

Amount made: 5.1 mg. LCMS m/z 520 [M+H]⁺, purity (UV/MS) 88/64.

### N-(3-chlorobenzyl)-1-(3-(4-(pyrrolidin-1-yl)piperidin-1-yl)propyl)-1H-indole-3-carboxamide C293

Amount made: 5.2 mg. LCMS m/z 479 [M+H]⁺, purity (UV/MS) 94/88.

### N-(3-chlorobenzyl)-1-(3-(methyl(2-(pyridin-2-yl)ethyl)amino)propyl)-1H-indole-3-carboxamide C294

Amount made: 8.2 mg. LCMS m/z 461 [M+H]⁺, purity (UV/MS) 88/68.

### N-(3-methylbenzyl)-1-(3-(2-phenyl)propylamino)propyl)-1H-indole-3-carboxamide C295

Amount made: 3.1 mg. LCMS m/z 440 [M+H]⁺, purity (UV/MS) 99/94.

### N-(3-methylbenzyl)-1-(3-(3-phenethyl-8-azabicyclo[3.2.1]octan-8-yl)propyl)-1H-indole-3-carboxamide C296

Amount made: 8.6 mg. LCMS m/z 520 [M+H]⁺, purity (UV/MS) 93/80.

### N-(3-methylbenzyl)-1-(3-(4-(pyrrolidin-1-yl)piperidin-1-yl)propyl)-1H-indole-3-carboxamide C297

Amount made: 13.2 mg. LCMS m/z 459 [M+H]⁺, purity (UV/MS) 100/85.

### N-(4-chlorobenzyl)-1-(3-(2-phenyl)propylamino)propyl)-1H-indole-3-carboxamide C298

Amount made: 2.9 mg. LCMS m/z 460 [M+H]⁺, purity (UV/MS) 100/97.

### N-(4-chlorobenzyl)-1-(3-(3-phenethyl-8-azabicyclo[3.2.1]octan-8-yl)propyl)-1H-indole-3-carboxamide C299

Amount made: 6.8 mg. LCMS m/z 540 [M+H]⁺, purity (UV/MS) 98/85.

### N-(4-chlorobenzyl)-1-(3-(4-(3-cyanopyridin-2-yl)-1,4-diazepan-1-yl)propyl)-1H-indole-3-carboxamide C300

Amount made: 8.2 mg. LCMS m/z 527 [M+H]⁺, purity (UV/MS) 73/64.

### N-(4-chlorobenzyl)-1-(3-(4-(4-chlorophenylthio)piperidin-1-yl)propyl)-1H-indole-3-carboxamide C301

Amount made: 6.5 mg. LCMS m/z 552 [M+H]⁺, purity (UV/MS) 80/68.

### N-(4-chlorobenzyl)-1-(3-(4-(4-fluorophenoxy)piperidin-1-yl)propyl)-1H-indole-3-carboxamide C302

Amount made: 8.8 mg. LCMS m/z 520 [M+H]⁺, purity (UV/MS) 100/100.

### N-(4-chlorobenzyl)-1-(3-(4-(pyrrolidin-1-yl)piperidin-1-yl)propyl)-1H-indole-3-carboxamide C303

Amount made: 5.1 mg. LCMS m/z 479 [M+H]⁺, purity (UV/MS) 100/100.

### N-(4-chlorobenzyl)-1-(3-(methyl(2-(pyridin-2-yl)ethyl)amino)propyl)-1H-indole-3-carboxamide C304

Amount made: 6.0 mg. LCMS m/z 461 [M+H]⁺, purity (UV/MS) 94/80.

### N-isobutyl-1-(3-(2-phenyl)propylamino)propyl)-1H-indole-3-carboxamide C305

Amount made: 3.6 mg. LCMS m/z 392 [M+H]⁺, purity (UV/MS) 100/100.

### N-isobutyl-1-(3-(3-phenethyl-8-azabicyclo[3.2.1]octan-8-yl)propyl)-1H-indole-3-carboxamide C306

Amount made: 4.8 mg. LCMS m/z 472 [M+H]⁺, purity (UV/MS) 85/75.

### N-isobutyl-1-(3-(4-(pyrrolidin-1-yl)piperidin-1-yl)propyl)-1H-indole-3-carboxamide C307

Amount made: 4.4 mg. LCMS m/z 411 [M+H]⁺, purity (UV/MS) 93/30.

### N-isobutyl-1-(3-(methyl(2-(pyridin-2-yl)ethyl)amino)propyl)-1H-indole-3-carboxamide C308

Amount made: 6.8 mg. LCMS m/z 393 [M+H]⁺, purity (UV/MS) 91/66.

### (1-(3-(2,3-dihydro-1H-inden-2-ylamino)propyl)-7-methoxy-1H-indol-3-yl)(phenyl)methanone C309

Amount made: 4.6 mg. LCMS m/z 425 [M+H]⁺, purity (UV/MS) 98/90.

### (1-(3-(3-(2-chlorobenzyl)piperidin-1-yl)propyl)-7-methoxy-1H-indol-3-yl)(cyclopropyl)methanone C310

Amount made: 13.2 mg. LCMS m/z 465 [M+H]⁺, purity (UV/MS) 100/90.

### (1-(3-(3-(2-chlorobenzyl)piperidin-1-yl)propyl)-7-methoxy-1H-indol-3-yl)(phenyl)methanone C311

Amount made: 3.2 mg. LCMS m/z 501 [M+H]⁺, purity (UV/MS) 100/90.

### (1-(3-(3-(2-chlorophenoxy)-8-azabicyclo[3.2.1]octan-8-yl)propyl)-7-methoxy-1H-indol-3-yl)(cyclopropyl)methanone C312

Amount made: 3.9 mg. LCMS m/z 493 [M+H]⁺, purity (UV/MS) 99/80.

### (1-(3-(3-(4-chlorophenethyl)-8-azabicyclo[3.2.1]octan-8-yl)propyl)-7-methoxy-1H-indol-3-yl)(cyclopropyl)methanone C313

Amount made: 6.1 mg. LCMS m/z 505 [M+H]⁺, purity (UV/MS) 98/90.

### (1-(3-(3-(4-chlorophenethyl)-8-azabicyclo[3.2.1]octan-8-yl)propyl)-7-methoxy-1H-indol-3-yl)(phenylmethanone C314

Amount made: 1.1 mg. LCMS m/z 541 [M+H]⁺, purity (UV/MS) 100/90.

### (1-(3-(3-(4-chlorophenoxy)-8-azabicyclo[3.2.1]octan-8-yl)propyl)-7-methoxy-1H-indol-3-yl)(cyclopropyl)methanone C315

Amount made: 5.9 mg. LCMS m/z 493 [M+H]⁺, purity (UV/MS) 99/90.

### (1-(3-(3-(4-chlorophenoxy)-8-azabicyclo[3.2.1]octan-8-yl)propyl)-7-methoxy-1H-indol-3-yl)(phenyl)methanone C316

Amount made: 5.4 mg. LCMS m/z 529 [M+H]⁺, purity (UV/MS) 80/60.

¹H NMR (400 MHz, CDCl₃) δ: 8.04-7.98 (m, 1H), 7.80-7.77 (m, 2H), 7.58-7.42 (m, 4H), 7.23-7.19 (m, 4H), 6.83-6.83 (m, 3H), 4.61-4.57 (m, 3H), 3.99 (s, 3H), 3.78-3.61 (m, 2H), 2.91-2.67 (m, 3H), 2.43-2.33 (m, 4H), 2.21-1.83 (m, 6H).

### (1-(3-(3-(4-chlorophenoxy)piperidin-1-yl)propyl)-7-methoxy-1H-indol-3-yl)(cyclopropyl)methanone C317

Amount made: 5.5 mg. LCMS m/z 467 [M+H]⁺, purity (UV/MS) 100/90.

### (1-(3-(3-(4-chlorophenoxy)piperidin-1-yl)propyl)-7-methoxy-1H-indol-3-yl)(phenyl)methanone C318

Amount made: 4.1 mg. LCMS m/z 503 [M+H]⁺, purity (UV/MS) 100/90.

### (1-(3-(3-(4-fluorophenoxy)-8-azabicyclo[3.2.1]octan-8-yl)propyl)-7-methoxy-1H-indol-3-yl)(phenyl)methanone C319

Amount made: 5.9 mg. LCMS m/z 513 [M+H]⁺, purity (UV/MS) 86/60.

### (1-(3-(3-benzoyl-7-methoxy-1H-indol-1-yl)propyl)piperidin-4-yl)(phenyl)methanone C320

Amount made: 2.1 mg. LCMS m/z 481 [M+H]⁺, purity (UV/MS) 100/70.

### (1-(3-(4-(2-(4-chloronaphthalen-1-yloxy)ethyl)piperazin-1-yl)propyl)-7-methoxy-1H-indol-3-yl)(cyclopropyl)methanone C321

Amount made: 1.4 mg. LCMS m/z 546 [M+H]⁺, purity (UV/MS) 100/100.

### (1-(3-(4-(2-(4-chlorophenoxy)ethyl)piperazin-1-yl)propyl)-7-methoxy-1H-indol-3-yl)(cyclopropyl)methanone C322

Amount made: 8.2 mg. LCMS m/z 496 [M+H]⁺, purity (UV/MS) 97/80.

### (1-(3-(4-(2-(4-chlorophenoxy)ethyl)piperazin-1-yl)propyl)-7-methoxy-1H-indol-3-yl)(phenyl)methanone C323

Amount made: 8.0 mg. LCMS m/z 532 [M+H]⁺, purity (UV/MS) 100/100.

### (1-(3-(4-(2-(4-chlorophenoxy)ethyl)piperidin-1-yl)propyl)-7-methoxy-1H-indol-3-yl)(cyclopropyl)methanone C324

Amount made: 3.9 mg. LCMS m/z 495 [M+H]⁺, purity (UV/MS) 100/90.

### (1-(3-(4-(2-chlorobenzyl)-1,4-diazepan-1-yl)propyl)-7-methoxy-1H-indol-3-yl)(cyclopropyl)methanone C325

Amount made: 3.8 mg. LCMS m/z 480 [M+H]⁺, purity (UV/MS) 98/80.

### (1-(3-(4-(2-chlorobenzyl)-1,4-diazepan-1-yl)propyl)-7-methoxy-1H-indol-3-yl)(phenyl)methanone C326

Amount made: 5.9 mg. LCMS m/z 516 [M+H]⁺, purity (UV/MS) 98/90.

### (1-(3-(4-(2-chlorophenoxy)piperidin-1-yl)propyl)-7-methoxy-1H-indol-3-yl)(cyclopropyl)methanone C327

Amount made: 3.0 mg. LCMS m/z 467 [M+H]⁺, purity (UV/MS) 94/70.

### (1-(3-(4-(2-chlorophenoxy)piperidin-1-yl)propyl)-7-methoxy-1H-indol-3-yl)(phenyl)methanone C328

Amount made, 8.6 mg. LCMS m/z 503 [M+H]⁺, purity (UV/MS) 92/50.

### (1-(3-(4-(3-chlorophenoxy)piperidin-1-yl)propyl)-7-methoxy-1H-indol-3-yl)(cyclopropyl)methanone C329

Amount made: 6.6 mg. LCMS m/z 467 [M+H]⁺, purity (UV/MS) 100/90.

### (1-(3-(4-(3-chlorophenoxy)piperidin-1-yl)propyl)-7-methoxy-1H-indol-3-yl)(phenyl)methanone C330

Amount made: 6.7 mg. LCMS m/z 503 [M+H]⁺, purity (UV/MS) 100/90.

### (1-(3-(4-(4-chlorophenoxy)piperidin-1-yl)propyl)-7-methoxy-1H-indol-3-yl)(cyclopropyl)methanone C331

Amount made: 4.4 mg. LCMS m/z 467 [M+H]⁺, purity (UV/MS) 97/80.

### (1-(3-(4-(4-chlorophenoxy)piperidin-1-yl)propyl)-7-methoxy-1H-indol-3-yl)(phenyl)methanone C332

Amount made: 6.3 mg. LCMS m/z 503 [M+H]⁺, purity (UV/MS) 80/50.

### (1-(3-(4-(4-fluorobenzyl)-1,4-diazepan-1-yl)propyl)-7-methoxy-1H-indol-3-yl)(phenyl)methanone C333

Amount made: 6.4 mg. LCMS m/z 500 [M+H]⁺, purity (UV/MS) 100/90.

### (1-(3-(4-(4-fluorophenoxy)piperidin-1-yl)propyl)-7-methoxy-1H-indol-3-yl)(phenyl)methanone C334

Amount made: 4.5 mg. LCMS m/z 487 [M+H]⁺, purity (UV/MS) 96/60.

### (1-(3-(4-(benzo[d]thiazol-2-yl)piperidin-1-yl)propyl)-7-methoxy-1H-indol-3-yl)(cyclopropyl)methanone C335

Amount made: 3.7 mg. LCMS m/z 474 [M+H]⁺, purity (UV/MS) 95/70.

### (1-(3-(4-(benzo[d]thiazol-2-yl)piperidin-1-yl)propyl)-7-methoxy-1H-indol-3-yl)(phenyl)methanone C336

Amount made: 7.6 mg. LCMS m/z 510 [M+H]⁺, purity (UV/MS) 99/70.

### (1-(3-(4-benzoylpiperidin-1-yl)propyl)-7-methoxy-1H-indol-3-yl)(cyclopropyl)methanone C337

Amount made: 3.7 mg. LCMS m/z 445 [M+H]⁺, purity (UV/MS) 97/70.

### (1-(3-(4-butylpiperidin-1-yl)propyl)-7-methoxy-1H-indol-3-yl)(cyclopropyl)methanone C338

Amount made: 5.1 mg. LCMS m/z 397 [M+H]⁺, purity (UV/MS) 100/90.

### (1-(3-(4-butylpiperidin-1-yl)propyl)-7-methoxy-1H-indol-3-yl)(phenyl)methanone C339

Amount made: 8.5 mg. LCMS m/z 433 [M+H]⁺, purity (UV/MS) 99/90.

### (1R,5S)-8-(3-(3-acetyl-7-methoxy-1H-indol-1-yl)propyl)-8-azabicyclo[3.2.1]octan-3-yl 3,4-dimethoxybenzoate C340

Amount made: 9.3 mg. LCMS m/z 521 [M+H]⁺, purity (UV/MS) 95/70.

### (7-methoxy-1-(3-(2-phenoxyethylamino)propyl)-1H-indol-3-yl)(phenyl)methanone C341

Amount made: 2.6 mg. LCMS m/z 429 [M+H]⁺, purity (UV/MS) 98/80.

### (7-methoxy-1-(3-(3-pentyl-8-azabicyclo[3.2.1]octan-8-yl)propyl)-1H-indol-3-yl)(phenyl)methanone C342

Amount made: 3.1 mg. LCMS m/z 473 [M+H]⁺, purity (UV/MS) 92/92.

### (7-methoxy-1-(3-(4-(2-methoxyphenyl)piperidin-1-yl)propyl)-1H-indol-3-yl)(Phenyl)methanone C343

Amount made: 7.7 mg. LCMS m/z 483 [M+H]⁺, purity (UV/MS) 95/70.

### (7-methoxy-1-(3-(4-(2-phenoxyethyl)piperazin-1-yl)propyl)-1H-indol-3-yl)(phenyl)methanone C344

Amount made: 7.5 mg. LCMS m/z 498 [M+H]⁺, purity (UV/MS) 100/90.

### (7-methoxy-1-(3-(4-(3-(pyridin-3-yl)-1,2,4-oxadiazol-5-yl)piperidin-1-yl)propyl)-1H-indol-3-yl)(phenyl)methanone C345

Amount made: 6.1 mg. LCMS m/z 522 [M+H]⁺, purity (UV/MS) 97/70.

### (7-methoxy-1-(3-(4-(3-(pyridin-4-yl)-1,2,4-oxadiazol-5-yl)piperidin-1-yl)propyl)-1H-indol-3-yl)(phenyl)methanone C346

Amount made: 8.4 mg. LCMS m/z 522 [M+H]⁺, purity (UV/MS) 95/60.

### (7-methoxy-1-(3-(4-phenethyl-1,4-diazepan-1-yl)propyl)-1H-indol-3-yl)(phenyl)methanone C347

Amount made: 7.0 mg. LCMS m/z 496 [M+H]⁺, purity (UV/MS) 98/90.

### 1-(1-(3-(2,3-dihydro-1H-inden-2-ylamino)propyl)-7-ethyl-1H-indol-3-yl)ethanone C348

Amount made: 2.3 mg. LCMS m/z 361 [M+H]⁺, purity (UV/MS) 99/90.

### 1-(1-(3-(2,3-dihydro-1H-inden-2-ylamino)propyl)-7-methoxy-1H-indol-3-yl)-2-phenylethanone C349

Amount made: 1.8 mg. LCMS m/z 439 [M+H]⁺, purity (UV/MS) 99/80.

### 1-(1-(3-(2,3-dihydro-1H-inden-2-ylamino)propyl)-7-methyl-1H-indol-3-yl)ethanone C350

Amount made: 3.0 mg. LCMS m/z 347 [M+H]⁺, purity (UV/MS) 93/80.

### 1-(1-(3-(3-(2-chlorobenzyl)piperidin-1-yl)propyl)-7 ethyl-1H-indol-3-yl)ethanone C351

Amount made: 2.4 mg. LCMS m/z 437 [M+H]⁺, purity (UV/MS) 100/90.

### 1-(1-(3-(3-(2-chlorobenzyl)piperidin-1-yl)propyl)-7-methoxy-1H-indol-3-yl)-2-phenylethanone C352

Amount made: 2.1 mg. LCMS m/z 515 [M+H]⁺, purity (UV/MS) 98/90.

### 1-(1-(3-(3-(2-chlorobenzyl)piperidin-1-yl)propyl)-7-methyl-1H-indol-3-yl)ethanone C353

Amount made: 3.2 mg. LCMS m/z 423 [M+H]⁺, purity (UV/MS) 100/80.

### 1-(1-(3-(3-(2-chlorobenzyl)pyrrolidin-1-yl)propyl)-7-methyl-1H-indol-3-yl)ethanone C354

Amount made: 0.4 mg. LCMS m/z 409 [M+H]⁺, purity (UV/MS) 98/90.

### 1-(1-(3-(3-(2-chlorophenoxy)-8-azabicyclo[3.2.1]octan-8-yl)propyl)-7-ethyl-1H-indol-3-yl)ethanone C355

Amount made: 2.0 mg. LCMS m/z 465 [M+H]⁺, purity (UV/MS) 97/80.

### 1-(1-(3-(3-(2-chlorophenoxy)-8-azabicyclo[3.2.1]octan-8-yl)propyl)-7-methoxy-1H-indol-3-yl)-2-phenylethanone C356

Amount made: 3.1 mg. LCMS m/z 543 [M+H]⁺, purity (UV/MS) 100/90.

### 1-(1-(3-(3-(2-chlorophenoxy)-8-azabicyclo[3.2.1]octan-8-yl)propyl)-7-methyl-1H-indol-3-yl)ethanone C357

Amount made: 0.3 mg. LCMS m/z 451 [M+H]⁺, purity (UV/MS) 99/90.

### 1-(1-(3-(3-(4-chlorophenethyl)-8-azabicyclo[3.2.1]octan-8-yl)propyl)-7-ethyl-1H-indol-3-yl)ethanone C358

Amount made: 4.3 mg. LCMS m/z 477 [M+H]⁺, purity (UV/MS) 93/70.

### 1-(1-(3-(3-(4-chlorophenethyl)-8-azabicyclo[3.2.1]octan-8-yl)propyl)-7-methyl-1H-indol-3-yl)ethanone C359

Amount made: 2.8 mg. LCMS m/z 463 [M+H]⁺, purity (UV/MS) 100/90.

### 1-(1-(3-(3-(4-chlorophenoxy)-8-azabicyclo[3.2.1]octan-8-yl)propyl)-7-methyl-1H-indol-3-yl)ethanone C360

Amount made: 0.8 mg. LCMS m/z 451 [M+H]⁺, purity (UV/MS) 99/80.

### 1-(1-(3-(3-(4-chlorophenoxy)piperidin-1-yl)-2-methyl)propyl)-7-methoxy-1H-indol-3-yl)ethanone C361

Amount made: 1.5 mg. LCMS m/z 455 [M+H]⁺, purity (UV/MS) 100/90.

### 1-(1-(3-(3-(4-chlorophenoxy)piperidin-1-yl)propyl)-7-ethyl-1H-indol-3-yl)ethanone C362

Amount made: 3.7 mg. LCMS m/z 439 [M+H]⁺, purity (UV/MS) 100/100.

### 1-(1-(3-(3-(4-chlorophenoxy)piperidin-1-yl)propyl)-7-methoxy-1H-indol-3-yl)-2-phenylethanone C363

Amount made: 2.7 mg. LCMS m/z 517 [M+H]⁺, purity (UV/MS) 100/100.

### 1-(1-(3-C3-(4-chlorophenoxy)piperidin-1-yl)propyl)-7-methyl-1H-indol-3-yl)ethanone C364

Amount made: 2.2 mg. LCMS m/z 425 [M+H]⁺, purity (UV/MS) 100/100.

### 1-(1-(3-(3-(4-fluorophenoxy)-8-azabicyclo[3.2.1]octan-8-yl)-2-methyl)propyl)-7-methoxy-1H-indol-3-yl)ethanone C365

Amount made: 0.6 mg. LCMS m/z 465 [M+H]⁺, purity (UV/MS) 79/60.

### 1-(1-(3-(3-(4-fluorophenoxy)-8-azabicyclo[3.2.1]octan-8-yl)propyl)-7-methoxy-1H-indol-3-yl)-2-phenylethanone C366

Amount made: 5.2 mg. LCMS m/z 527 [M+H]⁺, purity (UV/MS) 92/80.

### 1-(1-(3-(3-(4-fluorophenoxy)-8-azabicyclo[3.2.1]octan-8-yl)propyl)-7-methyl-1H-indol-3-yl)ethanone C367

Amount made: 4.1 mg. LCMS m/z 435 [M+H]⁺, purity (UV/MS) 98/70.

### 1-(1-(3-(3-(4-fluorophenyl)-3-hydroxy-8-azabicyclo[3.2.1]octan-8-yl)propyl)-7-methoxy-1H-indol-3-yl)ethanone C368

Amount made: 8.9 mg. LCMS m/z 451 [M+H]⁺, purity (UV/MS) 98/70.

### 1-(1-(3-(3-(cyclooropanecarbonyl)-7-methoxy-1H-indol-1-yl)propyl)piperidin-4-yl)indolin-2-one C369

Amount made: 1.6 mg. LCMS m/z 472 [M+H]⁺, purity (UV/MS) 97/90.

Amount made: 2.8 mg. LCMS m/z 494 [M+H]⁺, purity (UV/MS) 96/90.

### 1-(1-(3-(3-acetyl-7-bromo-2-methyl-1H-indol-1-yl)propyl)piperidin-4-yl)indolin-2-one C371

Amount made: 0.6 mg. LCMS m/z 508 [M+H]⁺, purity (UV/MS) 94/90.

### 1-(1-(3-(3-acetyl-7-chloro-1H-indol-1-yl)propyl)piperidin-4-yl)indolin-2-one C372

Amount made: 1.4 mg. LCMS m/z 450 [M+H]⁺, purity (UV/MS) 93/90.

### 1-(1-(3-(3-acetyl-7-ethyl-1H-indol-1-yl)propyl)piperidin-4-yl)indolin-2-one C373

Amount made: 0.5 mg. LCMS m/z 444 [M+H]⁺, purity (UV/MS) 90/90.

### 1-(1-(3-(3-acetyl-7-methoxy-1H-indol-1-yl)propyl)piperidin-4-yl)-1H-benzo[d]imidazol-2(3H)-one C374

Amount made: 8.4 mg. LCMS m/z 447 [M+H]⁺, purity (UV/MS) 99/80.

### 1-(1-(3-(3-acetyl-7-methyl-1H-indol-1-yl)propyl)piperidin-4-yl)indolin-2-one C375

Amount made: 1.9 mg. LCMS m/z 430 [M+H]⁺, purity (UV/MS) 87/90.

### 1-(1-(3-(4-(2-(4-chloronaphthalen-1-yloxy)ethyl)piperazin-1-yl)-2-methyl)propyl)-7-methoxy-1H-indol-3-yl)ethanone C376

Amount made: 0.2 mg. LCMS m/z 534 [M+H]⁺, purity (UV/MS) 100/90.

### 1-(1-(3-(4-(2-(4-chloronaphthalen-1-yloxy)ethyl)piperazin-1-yl)propyl)-7-ethyl-1H-indol-3-yl)ethanone C377

Amount made: 1.6 mg. LCMS m/z 518 [M+H]⁺, purity (UV/MS) 100/100.

### 1-(1-(3-(4-(2-(4-chloronaphthalen-1-yloxy)ethyl)piperazin-1-yl)propyl)-7-methoxy-1H-indol-3-yl)-2-phenylethanone C378

Amount made: 0.7 mg. LCMS m/z 596 [M+H]⁺, purity (UV/MS) 100/90.

### 1-(1-(3-(4-(2-(4-chloronaphthalen-1-yloxy)ethyl)piperazin-1-yl)propyl)-7-methyl-1H-indol-3-yl)ethanone C379

Amount made: 1.1 mg. LCMS m/z 504 [M+H]⁺, purity (UV/MS) 100/100.

### 1-(1-(3-(4-(2-(4-chlorophenoxy)ethyl)piperazin-1-yl)-2-methyl)propyl)-7-methoxy-1H-indol-3-yl)ethanone C380

Amount made: 5.4 mg. LCMS m/z 484 [M+H]⁺, purity (UV/MS) 100/90.

### 1-(1-(3-(4-(2-(4-chlorophenoxy)ethyl)piperazin-1-yl)propyl)-7-ethyl-1H-indol-3-yl)ethanone C381

Amount made: 7.9 mg. LCMS m/z 468 [M+H]⁺, purity (UV/MS) 100/90.

### 1-(1-(3-(4-(2-(4-chlorophenoxy)ethyl)piperazin-1-yl)propyl)-7-methoxy-1H-indol-3-yl)-2-phenylethanone C382

Amount made: 5.6 mg. LCMS m/z 546 [M+H]⁺, purity (UV/MS) 100/90.

### 1-(1-(3-(4-(2-(4-chlorophenoxy)ethyl)piperazin-1-yl)propyl)-7-methyl-1H-indol-3-yl)ethanone C383

Amount made: 5.3 mg. LCMS m/z 454 [M+H]⁺, purity (UV/MS) 98/90.

### 1-(1-(3-(4-(2-(4-chlorophenoxy)ethyl)piperidin-1-yl)-2-methyl)propyl)-7-methoxy-1H-indol-3-yl)ethanone C384

Amount made: 1.7 mg. LCMS m/z 483 [M+H]⁺, purity (UV/MS) 99/90.

### 1-(1-(3-(4-(2-(4-chlorophenoxy)ethyl)piperidin-1-yl)propyl)-7-ethyl-1H-indol-3-yl)ethanone C385

Amount made: 2.9 mg. LCMS m/z 467 [M+H]⁺, purity (UV/MS) 98/90.

### 1-(1-(3-(4-(2-(4-chlorophenoxy)ethyl)piperidin-1-yl)propyl)-7-methoxy-1H-indol-3-yl)-2-phenylethanone C386

Amount made: 0.9 mg. LCMS m/z 545 [M+H]⁺, purity (UV/MS) 97/90.

### 1-(1-(3-(4-(2-(4-chlorophenoxy)ethyl)piperidin-1-yl)propyl)-7-methyl-1H-indol-3-yl)ethanone C387

Amount made: 2.9 mg. LCMS m/z 453 [M+H]⁺, purity (UV/MS) 100/90.

### 1-(1-(3-(4-(2,3-dichlorophenyl)piperazin-1-yl)propyl)-7-methoxy-1H-indol-3-yl)ethanone C388

Amount made: 1.8 mg. LCMS m/z 460 [M+H]⁺, purity (UV/MS) 100/90.

### 1-(1-(3-(4-(2,4-dichlorobenzyl)piperazin-1-yl)-2-methyl)propyl)-7-methoxy-1H-indol-3-yl)ethanone C389

Amount made: 6.5 mg. LCMS m/z 488 [M+H]⁺, purity (UV/MS) 100/90.

### 1-(1-(3-(4-(2,4-dichlorobenzyl)piperazin-1-yl)propyl)-7-ethyl-1H-indol-3-yl)ethanone C390

Amount made: 2.5 mg. LCMS m/z 472 [M+H]⁺, purity (UV/MS) 100/90.

### 1-(1-(3-(4-(2,4-dichlorobenzyl)piperazin-1-yl)propyl)-7-methoxy-1H-indol-3-yl)-2-phenylethanone C391

Amount made: 6.4 mg. LCMS m/z 550 [M+H]⁺, purity (UV/MS) 100/90.

### 1-(1-(3-(4-(2,4-dichlorobenzyl)piperazin-1-yl)propyl)-7-methyl-1H-indol-3-yl)ethanone C392

Amount made: 3.4 mg. LCMS m/z 458 [M+H]⁺, purity (UV/MS) 100/100.

### 1-(1-(3-(4-(2,6-dimethylphenyl)piperazin-1-yl)propyl)-7-methoxy-1H-indol-3-yl)ethanone C393

Amount made: 9.2 mg. LCMS m/z 420 [M+H]⁺, purity (UV/MS) 97/60.

### 1-(1-(3-(4-(2-chlorobenzyl)-1,4-diazepan-1-yl)-2-methyl)propyl)-7-methoxy-1H-indol-3-yl)ethanone C394

Amount made: 4.0 mg. LCMS m/z 468 [M+H]⁺, purity (UV/MS) 99/90.

### 1-(1-(3-(4-(2-chlorobenzyl)-1,4-diazepan-1-yl)propyl)-7-ethyl-1H-indol-3-yl)ethanone C395

Amount made: 3.4 mg. LCMS m/z 452 [M+H]⁺, purity (UV/MS) 100/90.

### 1-(1-(3-(4-(2-chlorobenzyl)-1,4-diazepan-1-yl)propyl)-7-methoxy-1H-indol-3-yl)-2-phenylethanone C396

Amount made: 5.5 mg. LCMS m/z 530 [M+H]⁺, purity (UV/MS) 100/90.

### 1-(1-(3-(4-(2-chlorobenzyl)-1,4-diazepan-1-yl)propyl)-7-methyl-1H-indol-3-yl)ethanone C397

Amount made: 2.5 mg. LCMS m/z 438 [M+H]⁺, purity (UV/MS) 97/50.

### 1-(1-(3-(4-(2-chlorophenoxy)piperidin-1-yl)propyl)-7-methyl-1H-indol-3-yl)ethanone C398

Amount made: 6.2 mg. LCMS m/z 425 [M+H]⁺, purity (UV/MS) 100/90.

### 1-(1-(3-(4-(2-chlorophenyl)piperazin-1-yl)propyl)-7-methoxy-1H-indol-3-yl)ethanone C399

Amount made: 2.2 mg. LCMS m/z 426 [M+H]⁺, purity (UV/MS) 100/80.

### 1-(1-(3-(4-(2-methoxyphenyl)piperidin-1-yl)propyl)-7-methyl-1H-indol-3-yl)ethanone C400

Amount made: 8.6 mg. LCMS m/z 405 [M+H]⁺, purity (UV/MS) 99/80.

### 1-(1-(3-(4-(3-chloro-5-(trifluoromethyl)pyridin-2-yl)piperazin-1-yl)-2-methyl)propyl)-7-methoxy-1H-indol-3-yl)ethanone C401

Amount made: 5.4 mg. LCMS m/z 509 [M+H]⁺, purity (UV/MS) 100/100.

### 1-(1-(3-(4-(3-chloro-5-(trifluoromethyl)pyridin-2-yl)piperazin-1-yl)propyl)-7-ethyl-1H-indol-3-yl)ethanone C402

Amount made: 4.7 mg. LCMS m/z 493 [M+H]⁺, purity (UV/MS) 100/100.

### 1-(1-(3-(4-(3-chloro-5-(trifluoromethyl)pyridin-2-yl)piperazin-1-yl)propyl)-7-methoxy-1H-indol-3-yl)-2-phenylethanone C403

Amount made: 6.9 mg. LCMS m/z 571 [M+H]⁺, purity (UV/MS) 100/90.

### 1-(1-(3-(4-(3-chloro-5-(trifluoromethyl)pyridin-2-yl)piperazin-1-yl)propyl)-7-methoxy-1H-indol-3-yl)ethanone C404

Amount made: 8.0 mg. LCMS m/z 495 [M+H]⁺, purity (UV/MS) 98/90.

### 1-(1-(3-(4-(3-chloro-5-(trifluoromethyl)pyridin-2-yl)piperazin-1-yl)propyl)-7-methyl-1H-indol-3-yl)ethanone C405

Amount made: 6.2 mg. LCMS m/z 479 [M+H]⁺, purity (UV/MS) 98/80.

### 1-(1-(3-(4-(3-chlorophenoxy)piperidin-1-yl)-2-methyl)propyl)-7-methoxy-1H-indol-3-yl)ethanone C406

Amount made: 2.7 mg. LCMS m/z 455 [M+H]⁺, purity (UV/MS) 100/90.

### 1-(1-(3-(4-(3-chlorophenoxy)piperidin-1-yl)propyl)-7-ethyl-1H-indol-3-yl)ethanone C407

Amount made: 6.5 mg. LCMS m/z 439 [M+H]⁺, purity (UV/MS) 100/100.

### 1-(1-(3-(4-(3-chlorophenoxy)piperidin-1-yl)propyl)-7-methoxy-1H-indol-3-yl)-2-phenylethanone C408

Amount made: 4.6 mg. LCMS m/z 517 [M+H]⁺, purity (UV/MS) 100/100.

### 1-(1-(3-(4-(3-chlorophenoxy)piperidin-1-yl)propyl)-7-methyl-1H-indol-3-yl)ethanone C409

Amount made: 4.5 mg. LCMS m/z 425 [M+H]⁺, purity (UV/MS) 100/100.

### 1-(1-(3-(4-(4-chlorobenzyl)piperazin-1-yl)propyl)-7-methoxy-1H-indol-3-yl)ethanone C410

Amount made: 9.8 mg. LCMS m/z 440 [M+H]⁺, purity (UV/MS) 100/70.

### 1-(1-(3-(4-(4-chlorophenoxy)piperidin-1-yl)propyl)-7-methyl-1H-indol-3-yl)ethanone C411

Amount made: 5.5 mg. LCMS m/z 425 [M+H]⁺, purity (UV/MS) 99/80.

### 1-(1-(3-(4-(4-chlorophenylsulfonyl)piperidin-1-yl)propyl)-7-methoxy-1H-indol-3-yl)ethanone C412

Amount made: 3.6 mg. LCMS m/z 489 [M+H]⁺, purity (UV/MS) 100/90.

### 1-(1-(3-(4-(4-fluorobenzyl)-1,4-diazepan-1-yl)propyl)-7-methoxy-1H-indol-3-yl)-2-phenylethanone C413

Amount made: 3.4 mg. LCMS m/z 514 [M+H]⁺, purity (UV/MS) 92/80.

### 1-(1-(3-(4-(4-fluorobenzyl)-1,4-diazepan-1-yl)propyl)-7-methyl-1H-indol-3-yl)ethanone C414

Amount made: 3.5 mg. LCMS m/z 422 [M+H]⁺, purity (UV/MS) 98/70.

### 1-(1-(3-(4-(4-fluorophenoxy)piperidin-1-yl)propyl)-7-methyl-1H-indol-3-yl)ethanone C415

Amount made: 6.3 mg. LCMS m/z 409 [M+H]⁺, purity (UV/MS) 100/90.

¹H NMR (400 MHz, CDCl₃) δ 7.97 (d, 1H, J=8.0 Hz), 7.70 (s, 1H), 7.17 (t, 1H, J=8 Hz), 6.95 (m, 2H), 6.84 (m, 2H), 6.71 (d, 1H J=7.8 Hz), 4.47 (t, 2H, J=6.4 Hz), 4.24 (m, 1H), 3.94 (s, 3H), 2.73 (m, 1H), 2.49 (s, 3H), 2.32 (m, 4H), 2.05 (m, 4H), 1.84 (m, 2H).

### 1-(1-(3-(4-(4-fluorophenyl)piperazin-1-yl)propyl)-7-methoxy-1H-indol-3-yl)ethanone C416

Amount made: 7.4 mg. LCMS m/z 410 [M+H]⁺, purity (UV/MS) 98/60.

### 1-(1-(3-(4-(benzo[d]thiazol-2-yl)piperidin-1-yl)propyl)-7-bromo-1H-indol-3-yl)ethanone C417

Amount made: 2.4 mg. LCMS m/z 496 [M+H]⁺, purity (UV/MS) 100/80.

### 1-(1-(4-(benzo[d]thiazol-2-yl)piperidin-1-yl)propyl)-7-chloro-1H-indol-3-yl)ethanone C418

Amount made: 3.9 mg. LCMS m/z 452 [M+H]⁺, purity (UV/MS) 92/80.

### 1-(1-(3-(4-benzoylpiperidin-1-yl)propyl)-7-bromo-1H-indol-3-yl)ethanone C419

Amount made: 6.8 mg. LCMS m/z 467 [M+H]⁺, purity (UV/MS) 100/90.

### 1-(1-(3-(4-benzoylpiperidin-1-yl)propyl)-7-chloro-1H-indol-3-yl)ethanone C420

Amount made: 3.6 mg. LCMS m/z 423 [M+H]⁺, purity (UV/MS) 96/70.

### 1-(1-(3-(4-benzoylpiperidin-1-yl)propyl)-7-methyl-1H-indol-3-yl)ethanone C421

Amount made: 1.9 mg. LCMS m/z 403 [M+H]⁺, purity (UV/MS) 93/70.

### 1-(1-(3-(4-benzyl-4-hydroxypiperidin-1-yl)propyl)-7-methoxy-1H-indol-3-yl)ethanone C422

Amount made: 8.4 mg. LCMS m/z 421 [M+H]⁺, purity (UV/MS) 99/90.

### 1-(1-(3-(4-benzylpiperidin-1-yl)propyl)-7-methoxy-1H-indol-3-yl)ethanone C423

Amount made: 7.4 mg. LCMS m/z 405 [M+H]⁺, purity (UV/MS) 100/90.

### 1-(1-(3-(4-butylpiperidin-1-yl)propyl)-7-chloro-1H-indol-3-yl)ethanone C424

Amount made: 2.0 mg. LCMS m/z 375 [M+H]⁺, purity (UV/MS) 100/90.

### 1-(1-(3-(4-butylpiperidin-1-yl)propyl)-7-methyl-1H-indol-3-yl)ethanone C425

Amount made: 5.0 mg. LCMS m/z 355 [M+H]⁺, purity (UV/MS) 99/90.

### 1-(1-(3-(7-methoxy-3-(2-phenylacetyl)-1H-indol-1-yl)propyl)piperidin-4-yl)indolin-2-one C426

Amount made: 1.4 mg. LCMS m/z 522 [M+H]⁺, purity (UV/MS) 96/90.

### 1′-(3-(3-(cyclopropanecarbonyl)-7-methoxy-1H-indol-1-yl)propyl)spiro[chroman-2,4′-piperidin]-4-one C427

Amount made: 5.6 mg. LCMS m/z 473 [M+H]⁺, purity (UV/MS) 97/90.

### 1′-(3-(3-acetyl-7-bromo-1H-indol-1-yl)propyl)spiro[chroman-2,4′-piperidin]-4-one C428

Amount made: 2.1 mg. LCMS m/z 495 [M+H]⁺, purity (UV/MS) 100/90.

### 1′-(3-(3-acetyl-7-bromo-2-methyl-1H-indol-1-yl))propyl)spiro[chroman-2,4′-piperidin]-4-one C429

Amount made: 1.3 mg. LCMS m/z 509 [M+H]⁺, purity (UV/MS) 100/100.

### 1′-(3-(3-acetyl-7-chloro-1H-indol-1-yl)propyl)spiro[chroman-2,4′-piperidin]-4-one C430

Amount made: 5.4 mg. LCMS m/z 451 [M+H]⁺, purity (UV/NS) 100/90.

### 1′-(3-(3-acetyl-7-ethyl-1H-indol-1-yl)propyl)spiro[chroman-2,4′-piperidin]-4-one C431

Amount made: 4.6 mg. LCMS m/z 445 [M+H]⁺, purity (UV/MS) 100/90.

### 1-(3-(3-acetyl-7-methoxy-1H-indol-1-yl)-2-methyl)propyl)spiro[chroman-2,4′-piperidin]-4-one C432

Amount made: 0.4 mg. LCMS m/z 461 [M+H]⁺, purity (UV/MS) 98/90.

### 1-(3-(3-acetyl-7-methoxy-1H-indol-1-yl)propyl)-4-phenylpiperidine-4-carbonitrile C433

Amount made: 3.4 mg. LCMS m/z 416 [M+H]⁺, purity (UV/MS) 96/80.

### 1-(3-(3-acetyl-7-methoxy-1H-indol-1-yl)propyl)-N,N-diethylpiperidine-3-carboxamide C434

Amount made, 7.7 mg. LCMS m/z 414 [M+H]⁺, purity (UV/MS) 100/90.

### 1′-(3-(3-acetyl-7-methyl-1H-indol-1-yl)propyl)spiro[chroman-2,4′-piperidin]-4-one C435

Amount made: 2.8 mg. LCMS m/z 431 [M+H]⁺, purity (UV/MS) 100/90.

### 1′-(3-(3-benzoyl-7-methoxy-1H-indol-1-yl)propyl)spiro[chroman-2,4′-piperidin]-4-one C436

Amount made: 3.7 mg. LCMS m/z 509 [M+H]⁺, purity (UV/MS) 100/90.

### 1′-(3-(7-methoxy-3-(2-phenylacetyl)-1H-indo-1 yl)propyl)spiro[chroman-2,4′-piperidin]-4-one C437

Amount made: 1.9 mg. LCMS m/z 523 [M+H]⁺, purity (UV/MS) 100/90.

### 1-(4-chlorophenyl)-2-(8-(3-(3-(cyclopropanecarbonyl)-7-methoxy-1H-indol-1-yl)propyl)-8-azabicyclo[3.2.1]octan-3-yl)ethanone C438

Amount made: 6.0 mg. LCMS m/z 519 [M+H]⁺, purity (UV/MS) 98/80.

### 1-(4-chlorophenyl)-2-(8-(3-(7-methoxy-3-(2-phenylacetyl)-1H-indol-1-yl)propyl)-8-azabicyclo[3.2.1]octan-3-yl)ethanone C439

Amount made: 6.2 mg. LCMS m/z 569 [M+H]⁺, purity (UV/MS) 92/80.

### 1-(7-bromo-1-(3-(2,3-dihydro-1H-inden-2-ylamino)propyl)-1H-indol-3-yl)ethanone C440

Amount made: 4.1 mg. LCMS m/z 411 [M+H]⁺, purity (UV/MS) 100/90.

### 1-(7-bromo-1-(3-(2,3-dihydro-1H-inden-2-ylamino)propyl)-2-methyl-1H-indol-3-yl)ethanone C441

Amount made: 2.1 mg. LCMS m/z 425 [M+H]⁺, purity (UV/MS) 100/90.

### 1-(7-bromo-1-(3-(2-phenoxyethylamino)propyl)-1H-indol-3-yl)ethanone C442

Amount made: 3.7 mg. LCMS m/z 415 [M+H]⁺, purity (UV/MS) 100/90.

### 1-(7-bromo-1-(3-(3-(2-chlorobenzyl)piperidin-1-yl)propyl)-1H-indol-3-yl)ethanone C443

Amount made: 2.8 mg. LCMS m/z 487 [M+H]⁺, purity (UV/MS) 100/80.

### 1-(7-bromo-1-(3-(3-(2-chlorobenzyl)piperidin-1-yl)propyl)-2-methyl-1H-indol-3-yl)ethanone C444

Amount made: 0.9 mg. LCMS m/z 501 [M+H]⁺, purity (UV/MS) 98/90.

### 1-(7-bromo-1-(3-(3-(2-chlorobenzyl)pyrrolidin-1-yl)propyl)-2-methyl-1H-indol-3-yl)ethanone C445

Amount made: 0.8 mg. LCMS m/z 487 [M+H]⁺, purity (UV/MS) 98/90.

### 1-(7-bromo-1-(3-(3-(2-chlorophenoxy)-8-azabicyclo[3.2.1]octan-8-yl)propyl)-1H-indol-3-yl)ethanone C446

Amount made: 1.6 mg. LCMS m/z 515 [M+H]⁺, purity (UV/MS) 98/80.

### 1-(7-bromo-1-(3-(3-(2-chlorophenoxy)-8-azabicyclo[3.2.1]octan-8-yl)propyl)-2-methyl-1H-indol-3-yl)ethanone C447

Amount made: 1.6 mg. LCMS m/z 529 [M+H]⁺, purity (UV/MS) 100/80.

### 1-(7-bromo-1-(3-(3-(4-chlorophenethyl)-8-azabicyclo[3.2.1]octan-8-yl)propyl)-1H-indol-3-yl)ethanone C448

Amount made: 5.0 mg. LCMS m/z 527 [M+H]⁺, purity (UV/MS) 97/80.

### 1-(7-bromo-1-(3-(3-(4-chlorophenethyl)-8-azabicyclo[3.2.1]octan-8-yl)propyl)-2-methyl-1H-indol-3-yl)ethanone C449

Amount made: 3.5 mg. LCMS m/z 541 [M+H]⁺, purity (UV/MS) 97/80.

### 1-(7-bromo-1-(3-(3-(4-chlorophenoxy)piperidin-1-yl)propyl)-1H-indol-3-yl)ethanone C450

Amount made: 3.1 mg. LCMS m/z 489 [M+H]⁺, purity (UV/MS) 100/90.

### 1-(7-bromo-1-(3-(3-(4-chlorophenoxy)piperidin-1-yl)propyl)-2-methyl-1H-indol-3-yl)ethanone C451

Amount made: 0.4 mg. LCMS m/z 503 [M+H]⁺, purity (UV/MS) 100/70.

### 1-(7-bromo-1-(3-(3-(4-fluorophenoxy)-8-azabicyclo[3.2.1]octan-8-yl)propyl)-1H-indol-3-yl)ethanone C452

Amount made: 3.7 mg. LCMS m/z 499 [M+H]⁺, purity (UV/MS) 87/60.

### 1-(7-bromo-1-(3-(3-(4-fluorophenoxy)-8-azabicyclo[3.2.1]octan-8-yl)propyl)-2-methyl-1H-indol-3-yl)ethanone C453

Amount made: 1.7 mg. LCMS m/z 513 [M+H]⁺, purity (UV/MS) 94/90.

### 1-(7-bromo-1-(3-(3-pentyl-8-azabicyclo[3.2.1]octan-8-yl)propyl)-1H-indol-3-yl)ethanone C454

Amount made: 2.6 mg. LCMS m/z 459 [M+H]⁺, purity (UV/MS) 91/80.

### 1-(7-bromo-1-(3-(3-phenethyl-8-azabicyclo[3.2.1]octan-8-yl)propyl)-1H-indol-3-yl)ethanone C455

Amount made: 3.9 mg. LCMS m/z 493 [M+H]⁺, purity (UV/MS) 85/70.

### 1-(7-bromo-1-(3-(4-(2-(4-chlorophenoxy)ethyl)piperazin-1-yl)propyl)-1H-indol-3-yl)ethanone C456

Amount made: 3.2 mg. LCMS m/z 518 [M+H]⁺, purity (UV/MS) 100/100.

### 1-(7-bromo-1-(3-(4-(2-(4-chlorophenoxy)ethyl)piperazin-1-yl)propyl)-2-methyl-1H-indol-3-yl)ethanone C457

Amount made: 5.7 mg. LCMS m/z 532 [M+H]⁺, purity (UV/MS) 100/90.

### 1-(7-bromo-1-(3-(4-(2-(4-chlorophenoxy)ethyl)piperidin-1-yl)propyl)-2-methyl-1H-indol-3-yl)ethanone C458

Amount made: 3.1 mg. LCMS m/z 531 [M+H]⁺, purity (UV/MS) 100/80.

### 1-(7-bromo-1-(3-(4-(2-chlorobenzyl)-1,4-diazepan-1-yl)propyl)-1H-indol-3-yl)ethanone C459

Amount made: 3.4 mg. LCMS m/z 502 [M+H]⁺, purity (UV/MS) 98/90.

### 1-(7-bromo-1-(3-(4-(2-chlorobenzyl)-1,4-diazepan-1-yl)propyl)-2-methyl-1H-indol-3-yl)ethanone C460

Amount made: 3.3 mg. LCMS m/z 516 [M+H]⁺, purity (UV/MS) 98/90.

### 1-(7-bromo-1-(3-(4-(2-chlorophenoxy)piperidin-1-yl propyl)-1H-indol-3-yl)ethanone C461

Amount made: 3.3 mg. LCMS m/1489 [M+H]⁺, purity (UV/MS) 96/80.

### 1-(7-bromo-1-(3-C4-(2-methoxyphenol)piperidin-1-yl)propyl)-1H-indol-3-yl)ethanone C462

Amount made: 5.4 mg. LCMS m/z 469 [M+H]⁺, purity (UV/MS) 95/60.

### 1-(7-bromo-1-(3-(4-(2-phenoxyethyl)piperazin-1-yl)propyl)-1H-indol-3-yl)ethanone C463

Amount made. 8.8 mg. LCMS m/z 484 [M+H]⁺, purity (UV/MS) 100/100.

### 1-(7-bromo-1-(3-(4-(3-(pyridin-3-yl)-1,2,4-oxadiazol-5-yl)piperidin-1-yl)propyl)-1H-indol-3-yl)ethanone C464

Amount made: 8.0 mg. LCMS m/z 508 [M+H]⁺, purity (UV/MS) 100/90.

### 1-(7-bromo-1-(3-(4-(3-(pyridin-4-yl)-1,2,4-oxadiazol-5-yl)piperidin-1-yl)propyl)-1H-indol-3-yl)ethanone C465

Amount made: 0.6 mg. LCMS m/z 508 [M+H]⁺, purity (UV/MS) 100/70.

### 1-(7-bromo-1-(3-(4-(3-chlorophenoxy)piperidin-1-yl)propyl)-1H-indol-3-yl)ethanone C466

Amount made: 5.9 mg. LCMS m/z 489 [M+H]⁺, purity (UV/MS) 100/90.

### 1-(7-bromo-1-(3-(4-(3-chlorophenoxy)piperidin-1-yl)propyl)-2-methyl-1H-indol-3-yl)ethanone C467

Amount made: 4.9 mg. LCMS m/z 503 [M+H]⁺, purity (UV/MS) 100/90.

### 1-(7-bromo-1-(3-(4-(4-chlorophenoxy)piperidin-1-yl)propyl)-1H-indol-3-yl)ethanone C468

Amount made: 4.6 mg. LCMS m/z 489 [M+H]⁺, purity (UV/MS) 94/80.

### 1-(7-bromo-1-(3-(4-(4-fluorobenzyl)-1,4-diazepan-1-yl)propyl)-1H-indol-3-yl)ethanone C469

Amount made: 4.4 mg. LCMS m/z 486 [M+H]⁺, purity (UV/MS) 96/70.

### 1-(7-bromo-1-(3-(4-(4-fluorobenzyl)-1,4-diazepan-1-yl)propyl)-2-methyl-1H-indol-3-yl)ethanone C470

Amount made: 3.3 mg. LCMS m/z 500 [M+H]⁺, purity (UV/MS) 98/80.

### 1-(7-bromo-1-(3-(4-butylpiperidin-1-yl)propyl)-1H-indol-3-yl)ethanone C471

Amount made: 4.9 mg. LCMS m/z 419 [M+H]⁺, purity (UV/MS) 100/90.

### 1-(7-bromo-1-(3-(4-phenethyl-1,4-diazepan-1-yl)propyl)-1H-indol-3-yl)ethanone C472

Amount made: 6.2 mg. LCMS m/z 482 [M+H]⁺, purity (UV/MS) 100/90.

### 1-(7-bromo-2-methyl-1-(3-(2-phenoxyethylamino)propyl)-1H-indol-3-yl)ethanone C473

Amount made: 2.3 mg. LCMS m/z 429 [M+H]⁺, purity (UV/MS) 100/90.

### 1-(7-bromo-2-methyl-1-(3-(3-phenethyl-8-azabicyclo[3.2.1]octan-8-yl)propyl)-1H-indol-3-yl)ethanone C474

Amount made: 3.0 mg. LCMS m/z 507 [M+H]⁺, purity (UV/MS) 95/80.

### 1-(7-bromo-2-methyl-1-(3-(4-(2-phenoxyethyl)piperazin-1-yl)propyl)-1H-indol-3-yl)ethanone C475

Amount made: 6.4 mg. LCMS m/z 498 [M+H]⁺, purity (UV/MS) 100/90.

### 1-(7-bromo-2-methyl-1-(3-(4-(3-(pyridin-3-yl)-1,2,4-oxadiazol-5-yl)piperidin-1-yl)propyl)-1H-indol-3-yl)ethanone C476

Amount made: 4.6 mg. LCMS m/z 522 [M+H]⁺, purity (UV/MS) 98/90.

### 1-(7-bromo-2-methyl-1-(3-(4-(3-(pyridin-4-yl)-1,2,4-oxadiazol-5-yl)piperidin-1-yl)propyl) 1H-indol-3-yl)ethanone C477

Amount made: 5.7 mg. LCMS m/z 522 [M+H]⁺, purity (UV/MS) 99/80.

### 1-(7-bromo-2-methyl-1-(3-(4-phenethyl-1,4-diazepan-1-yl)propyl)-1H-indol-3-yl)ethanone C478

Amount made: 3.9 mg. LCMS m/z 496 [M+H]⁺, purity (UV/MS) 100/90.

### 1-(7-chloro-1-(3-(2,3-dihydro-1H-inden-2-ylamino)propyl)-1H-indol-3-yl)ethanone C479

Amount made: 3.0 mg. LCMS m/z 367 [M+H]⁺, purity (UV/MS) 100/90.

### 1-(7-chloro-1-(3-(2-phenoxyethylamino)propyl)-1H-indol-3-yl)ethanone C480

Amount made: 2.5 mg. LCMS m/z 371 [M+H]⁺, purity (UV/MS) 100/90.

### 1-(7-chloro-1-(3-(3-(2-chlorobenzyl)piperidin-1-yl)propyl)-1H-indol-3-yl)ethanone C481

Amount made: 3.6 mg. LCMS m/z 443 [M+H]⁺, purity (UV/MS) 100/70.

### 1-(7-chloro-1-(3-(3-(2-chlorobenzyl)pyrrolidin-1-yl)propyl)-1H-indol-3-yl)ethanone C482

Amount made: 1.0 mg. LCMS m/z 429 [M+H]⁺, purity (UV/MS) 100/90.

### 1-(7-chloro-1-(3-(3-(2-chlorophenoxy)-8-azabicyclo[3.2.1]octan-8-yl)propyl)-1H-indol-3-yl)ethanone C483

Amount made: 2.3 mg. LCMS m/z 471 [M+H]⁺, purity (UV/MS) 100/80.

### 1-(7-chloro-1-(3-(3-(4-chlorophenethyl)-8-azabicyclo[3.2.1]octan-8-yl)propyl)-1H-indol-3-yl)ethanone C484

Amount made: 4.5 mg. LCMS m/z 483 [M+H]⁺, purity (UV/MS) 97/80.

### 1-(7-chloro-1-(3-(3-(4-chlorophenoxy)-8-azabicyclo[3.2.1]octan-8-yl)propyl)-1H-indol-3-yl)ethanone C485

Amount made: 2.0 mg. LCMS m/z 471 [M+H]⁺, purity (UV/MS) 95/90.

### 1-(7-chloro-1-(3-(3-(4-chlorophenoxy)piperidin-1-yl)propyl)-1H-indol-3-yl)ethanone C486

Amount made: 4.0 mg. LCMS m/z 445 [M+H]⁺, purity (UV/MS) 100/100.

### 1-(7-chloro-1-(3-(3-(4-fluorophenoxy)-8-azabicyclo[3.2.1]octan-8-yl)propyl)-1H-indol-3-yl)ethanone C487

Amount made: 2.8 mg. LCMS m/z 455 [M+H]⁺, purity (UV/MS) 80/50.

### 1-(7-chloro-1-(3-(3-pentyl-8-azabicyclo[3.2.1]octan-8-yl)propyl)-1H-indol-3-yl)ethanone C488

Amount made: 2.2 mg. LCMS m/z 415 [M+H]⁺, purity (UV/MS) 98/96.

### 1-(7-chloro-1-(3-(3-phenethyl-8-azabicyclo[3.2.1]octan-8-yl)propyl)-1H-indol-3-yl)ethanone C489

Amount made: 3.4 mg. LCMS m/z 449 [M+H]⁺, purity (UV/MS) 100/80.

### 1-(7-chloro-1-(3-(4-(2-(4-chlorophenoxy)ethyl)piperazin-1-yl)propyl)-1H-indol-3-yl)ethanone C490

Amount made: 6.1 mg. LCMS m/z 474 [M+H]⁺, purity (UV/MS) 100/100.

### 1-(7-chloro-1-(3-(4-(2-(4-chlorophenoxy)ethyl)piperidin-1-yl)propyl)-1H-indol-3-yl)ethanone C491

Amount made: 2.4 mg. LCMS m/z 473 [M+H]⁺, purity (UV/MS) 98/80.

### 1-(7-chloro-1-(3-(4-(2-chlorobenzyl)-1,4-diazepan-1-yl)propyl)-1H-indol-3-yl)ethanone C492

Amount made: 3.4 mg. LCMS m/z 458 [M+H]⁺, purity (UV/MS) 98/80.

### 1-(7-chloro-1-(3-(4-(2-chlorophenoxy)piperidin-1-yl)propyl)-1H-indol-3-yl)ethanone C493

Amount made: 2.2 mg. LCMS m/z 445 [M+H]⁺, purity (UV/MS) 91/70.

### 1-(7-chloro-1-(3-(4-(2-methoxyphenyl)piperidin-1-yl)propyl)-1H-indol-3-yl)ethanone C494

Amount made: 3.1 mg. LCMS m/z 425 [M+H]⁺, purity (UV/MS) 95/70.

### 1-(7-chloro-1-(3-(4-(2-phenoxyethyl)piperazin-1-yl)propyl)-1H-indol-3-yl)ethanone C495

Amount made: 7.5 mg. LCMS m/z 440 [M+H]⁺, purity (UV/MS) 100/90.

### 1-(7-chloro-1-(3-(4-(3-(pyridin-3-yl)-1,2,4-oxadiazol-5-yl)piperidin-1-yl)propyl)-1H-indol-3-yl)ethanone C496

Amount made: 6.0 mg. LCMS m/z 464 [M+H]⁺, purity (UV/MS) 98/80.

### 1-(7-chloro-1-(3-(4-(3-(pyridin-4-yl)-1,2,4-oxadiazol-5-yl)piperidin-1-yl)propyl)-1H-indol-3-yl)ethanone C497

Amount made: 8.3 mg. LCMS m/z 464 [M+H]⁺, purity (UV/MS) 100/90.

### 1-(7-chloro-1-(3-(4-(3-chlorophenoxy)piperidin-1-yl)propyl)-1H-indol-3-yl)ethanone C498

Amount made: 8.3 mg. LCMS m/z 445 [M+H]⁺, purity (UV/MS) 100/80.

### 1-(7-chloro-1-(3-(4-(4-chlorophenoxy)piperidin-1-yl)propyl)-1H-indol-3-yl)ethanone C499

Amount made: 1.0 mg. LCMS m/z 445 [M+H]⁺, purity (UV/MS) 88/80.

### 1-(7-chloro-1-(3-(4-(4-fluorobenzyl)-1,4-diazepan-1-yl)propyl)-1H-indol-3-yl)ethanone C500

Amount made: 3.6 mg. LCMS m/z 442 [M+H]⁺, purity (UV/MS) 98/90.

### 1-(7-chloro-1-(3-(4-(4-fluorophenoxy)piperidin-1-yl)propyl)-1H-indol-3-yl)ethanone C501

Amount made: 3.7 mg. LCMS m/z 429 [M+H]⁺, purity (UV/MS) 90/80.

### 1-(7-chloro-1-(3-(4-phenethyl-1,4-diazepan-1-yl)propyl)-1H-indol-3-yl)ethanone C502

Amount made: 4.9 mg. LCMS m/z 438 [M+H]⁺, purity (UV/MS) 100/90.

### 1-(7-ethyl-1-(3-(2-phenoxyethylamino)propyl)-1H-indol-3-yl)ethanone C503

Amount made: 2.3 mg. LCMS m/z 365 [M+H]⁺, purity (UV/MS) 99/90.

### 1-(7-ethyl-1-(3-(3-(4-fluorophenoxy)-8-azabicyclo[3.2.1]octan-8-yl)propyl)-1H-indol-3-yl)ethanone C504

Amount made: 1.9 mg. LCMS m/z 449 [M+H]⁺, purity (UV/MS) 98/70.

### 1-(7-ethyl-1-(3-(3-phenethyl-8-azabicyclo[3.2.1]octan-8-yl)propyl)-1H-indol-3-yl)ethanone C505

Amount made: 3.4 mg. LCMS m/z 443 [M+H]⁺, purity (UV/MS) 92/80.

### 1-(7-ethyl-1-(3-(4-(2-phenoxyethyl)piperazin-1-yl)propyl)-1H-indol-3-yl)ethanone C506

Amount made: 8.8 mg. LCMS m/z 434 [M+H]⁺, purity (UV/MS) 100/90.

### 1-(7-ethyl-1-(3-(4-(3-(pyridin-3-yl)-1,2,4-oxadiazol-5-yl)piperidin-1-yl)propyl)-1H-indol-3-yl)ethanone C507

Amount made: 2.4 mg. LCMS m/z 458 [M+H]⁺, purity (UV/MS) 100/90.

### 1-(7-ethyl-1-(3-(4-(3-(pyridin-4-yl)-1,2,4-oxadiazol-5-yl)piperidin-1-yl)propyl)-1H-indol-3-yl)ethanone C508

Amount made: 4.8 mg. LCMS m/z 458 [M+H]⁺, purity (UV/MS) 100/80.

### 1-(7-ethyl-1-(3-(4-(4-fluorobenzyl)-1,4-diazepan-1-yl)propyl)-1H-indol-3-yl)ethanone C509

Amount made: 2.6 mg. LCMS m/z 436 [M+H]⁺, purity (UV/MS) 94/80.

### 1-(7-ethyl-1-(3-(4-phenethyl-1,4-diazepan-1-yl)propyl)-1H-indol-3-yl)ethanone C510

Amount made: 4.2 mg. LCMS m/z 432 [M+H]⁺, purity (UV/MS) 97/80.

### 1-(7-methoxy-1-(2-methyl-1-(2-phenoxyethylamino)propyl)-1H-indol-3-yl)ethanone C511

Amount made: 0.3 mg. LCMS m/z 381 [M+H]⁺, purity (UV/MS) 98/90.

### 1-(7-methoxy-1-(2-methyl-3-(3-phenethyl-8-azabicyclo[3.2.1]octan-8-yl)propyl)-1H-indol-3-yl)ethanone C512

Amount made: 1.0 mg. LCMS m/z 459 [m+H]⁺, purity (UV/MS) 80/50.

### 1-(7-methoxy-1-(2-methyl-3-(4-(2-phenoxyethyl)piperazin-1-yl)propyl)-1H-indol-3-yl)ethanone C513

Amount made: 5.8 mg. LCMS m/z 450 [M+H]⁺, purity (UV/MS) 100/90.

### 1-(7-methoxy-1-(2-methyl-3-(4-(3-(pyridin-3-yl)-1,2,4-oxadiazol-5-yl)piperidin-1-yl)propyl)-1H-indol-3-yl)ethanone C514

Amount made: 4.6 mg. LCMS m/z 474 [M+H]⁺, purity (UV/MS) 100/90.

### 1-(7-methoxy-1-(2-methyl-3-(4-(3-(pyridin-4-yl)-1,2,4-oxadiazol-5-yl)piperidin-1-yl)propyl)-1H-indol-3-yl)ethanone C515

Amount made: 6.1 mg. LCMS m/z 474 [M+H]⁺, purity (UV/MS) 100/80.

### 1-(7-methoxy-1-(2-methyl-3-(4-phenethyl-1,4-diazepan-1-yl)propyl)-1H-indol-3-yl)ethanone C516

Amount made: 3.5 mg. LCMS m/z 448 [M+H]⁺, purity (UV/MS) 100/90.

### 1-(7-methoxy-1-(3-(2-phenoxyethylamino)propyl)-1H-indol-3-yl)-2-phenylethanone C517

Amount made: 2.9 mg. LCMS m/z 443 [M+H]⁺, purity (UV/MS) 99/70.

### 1-(7-methoxy-1-(3-(3-pentyl-8-azabicyclo[3.2.1]octan-8-yl)propyl)-1H-indol-3-yl)ethanone C518

Amount made: 7.1 mg. LCMS m/z 411 [M+H]⁺, purity (UV/MS) 97/80.

### 1-(7-methoxy-1-(3-(3-phenethyl-8-azabicyclo[3.2.1]octan-8-yl)propyl)-1H-indol-3-yl)-2-phenylethanone C519

Amount made: 3.6 mg. LCMS m/z 521 [M+H]⁺, purity (UV/MS) 96/90.

### 1-(7-methoxy-1-(3-(4-((tetrahydrofuran-2-yl)methyl)piperazin-1-yl)propyl)-1H-indol-3-yl)ethanone C520

Amount made: 5.4 mg. LCMS m/z 400 [M+H]⁺, purity (UV/MS) 81/50.

### 1-(7-methoxy-1-(3-(4-(2-nitro-4-(trifluoromethyl)phenyl)piperazin-1-yl)propyl)-1H-indol-3-yl)ethanone C521

Amount made: 8.6 mg. LCMS m/z 505 [M+H]⁺, purity (UV/MS) 84/80.

### 1-(7-methoxy-1-(3-(4-(2-phenoxyethyl)piperazin-1-yl)propyl)-1H-indol-3-yl)-2-phenylethanone C522

Amount made: 3.4 mg. LCMS m/z 512 [M+H]⁺, purity (UV/MS) 100/90.

### 1-(7-methoxy-1-(3-(4-(3-(pyridin-3-yl)-1,2,4-oxadiazol-5-yl)piperidin-1-yl)propyl)-1H-indol-3-yl)-2-phenylethanone C523

Amount made: 6.3 mg. LCMS m/z 536 [M+H]⁺, purity (UV/MS) 100/80.

### 1-(7-methoxy-1-(3-(4-(3-(pyridin-4-yl)-1,24-oxadiazol-5-yl)piperidin-1-yl)propyl)-1H-indol-3-yl)-2-phenylethanone C524

Amount made: 6.8 mg. LCMS m/z 536 [M+H]⁺, purity (UV/MS) 99/80.

### 1-(7-methoxy-1-(3-(4-(4-(trifluoromethyl)phenyl)piperazin-1-yl)propyl)-1H-indol-3-yl)ethanone C525

Amount made: 7.4 mg. LCMS m/z 460 [M+H]⁺, purity (UV/MS) 95/90.

### 1-(7-methoxy-1-(3-(4-morpholinopiperidin-1-yl)propyl)-1H-indol-3-yl)ethanone C526

Amount made: 2.4 mg. LCMS m/z 400 [M+H]⁺, purity (UV/MS) 89/60.

### 1-(7-methoxy-1-(3-(octahydroisoquinolin-2(1H)-yl)propyl)-1H-indol-3-yl)ethanone C527

Amount made: 8.4 mg. LCMS m/z 369 [M+H]⁺, purity (UV/MS) 99/90.

### 1-(7-methyl-1-(3-(2-phenoxyethylamino)propyl)-1H-indol-3-yl)ethanone C528

Amount made: 3.3 mg. LCMS m/z 351 [M+H]⁺, purity (UV/MS) 100/90.

### 1-(7-methyl-1-(3-(3-pentyl-8-azabicyclo[3.2.1]octan-8-yl)propyl)-1H-indol-3-yl)ethanone C529

Amount made: 3.0 mg. LCMS m/z 395 [M+H]⁺, purity (UV/MS) 82/70.

### 1-(7-methyl-1-(3-(3-phenethyl-8-azabicyclo[3.2.1]octan-8-yl)propyl)-1H-indol-3-yl)ethanone C530

Amount made: 3.5 mg. LCMS m/z 429 [M+H]⁺, purity (UV/MS) 98/90.

### 1-(7-methyl-1-(3-(4-(2-phenoxyethyl)piperazin-1-yl)propyl)-1H-indol-3-yl)ethanone C531

Amount made: 4.8 mg. LCMS m/z 420 [M+H]⁺, purity (UV/MS) 100/90.

### 1-(7-methyl-1-(3-(4-(3-(pyridin-3-yl)-1,2,4-oxadiazol-5-yl)piperidin-1-yl)propyl)-1H-indol-3-yl)ethanone C532

Amount made: 4.7 mg. LCMS m/z 444 [M+H]⁺, purity (UV/MS) 100/100.

### 1-(7-methyl-1-(3-(4-(3-(pyridin-4-yl) 1,2,4-oxadiazol-5-yl)piperidin-1-yl)propyl)-1H-indol-3-yl)ethanone C533

Amount made: 5.3 mg. LCMS m/z 444 [M+H]⁺, purity (UV/MS) 100/90.

### 1-(7-methyl-1-(3-(4-phenethyl-1,4-diazepan-1-yl)propyl)-1H-indol-3-yl)ethanone C534

Amount made: 5.1 mg. LCMS m/z 418 [M+H]⁺, purity (UV/MS) 97/70.

### 2-(4-(3-(3-acetyl-7-methoxy-1H-indol-1-yl)propyl)piperazin-1-yl)-1-morpholinoethanone C535

Amount made: 8.5 mg. LCMS m/z 443 [M+H]⁺, purity (UV/MS) 93/70.

### 2-(8-(3-(3-(cyclopropanecarbonyl)-7-methoxy-1H-indol-1-yl)propyl)-8-azabicyclo[3.2.1]octan-3-yl)-1-phenylethanone C536

Amount made: 4.6 mg. LCMS m/z 485 [M+H]⁺, purity (UV/MS) 94/70.

### 2-(8-(3-(3-acetyl-7-bromo-1H-indol-1-yl)propyl)-8-azabicyclo[3.2.1]octan-3-yl)-1-(4-chlorophenyl)ethanone C537

Amount made: 5.5 mg. LCMS m/z 541 [M+H]⁺, purity (UV/MS) 98/90.

### 2-(8-(3-(3-acetyl-7-bromo-1H-indol-1-yl)propyl)-8-azabicyclo[3.2.1]octan-3-yl)-1-phenylethanone C538

Amount made: 4.2 mg. LCMS m/z 507 [M+H]⁺, purity (UV/MS) 91/60.

### 2-(8-(3-(3-acetyl-7-bromo-2-methyl-1H-indol-1-yl)propyl)-8-azabicyclo[3.2.1]octan-3-yl)-1-(4-chlorophenyl)ethanone C539

Amount made: 3.9 mg. LCMS m/z 555 [M+H]⁺, purity (UV/MS) 98/70.

### 2-(8-(3-(3-acetyl-7-chloro-1H-indol-1-yl)propyl)-8-azabicyclo[3.2.1]octan-3-yl)-1-(4-chlorophenyl)ethanone C540

Amount made: 4.0 mg. LCMS m/z 497 [M+H]⁺, purity (UV/MS) 100/90.

### 2-(8-(3-(3-acetyl-7-chloro-1H-indol-1-yl)propyl)-8-azabicyclo[3.2.1]octan-3-yl)-1-phenylethanone C541

Amount made: 4.2 mg. LCMS m/z 463 [M+H]⁺, purity (UV/MS) 91/70.

### 2-(8-(3-(3-acetyl-7-ethyl-1H-indol-1-yl)propyl)-8-azabicyclo[3.2.1]octan-3-yl)-1-(4-chlorophenyl)ethanone C542

Amount made: 3.6 mg. LCMS m/z 491 [M+H]⁺, purity (UV/MS) 94/60.

### 2-(8-(3-(3-acetyl-7-methoxy-1H-indol-1-yl)-2-methyl)propyl)-8-azabicyclo[3.2.1]octan-3-yl)-1-(4-chlorophenyl)ethanone C543

Amount made: 5.1 mg. LCMS m/z 507 [M+H]⁺, purity (UV/MS) 96/90.

### 2-(8-(3-(3-acetyl-7-methyl-1H-indol-1-yl)propyl)-8-azabicyclo[3.2.1]octan-3-yl)-1-(4-chlorophenyl)ethanone C544

Amount made: 3.3 mg. LCMS m/z 477 [M+H]⁺, purity (UV/MS) 98/70.

### 2-(8-(3-(3-acetyl-7-methyl-1H-indol-1-yl)propyl)-8-azabicyclo[3.2.1]octan-3-yl)-1-phenylethanone C545

Amount made: 2.1 mg. LCMS m/z 443 [M+H]⁺, purity (UV/MS) 99/80.

### 2-(8-(3-(3-benzoyl-7-methoxy-1H-indol-1-yl)propyl)-8-azabicyclo[3.2.1]octan-3-yl)-1-(4-chlorophenyl)ethanone C546

Amount made: 7.0 mg. LCMS m/z 555 [M+H]⁺, purity (UV/MS) 96/70.

### 2-(8-(3-(3-benzoyl-7-methoxy-1H-indol-1-yl)propyl)-8-azabicyclo[3.2.1]octan-3-yl)-1-phenylethanone C547

Amount made: 5.7 mg. LCMS m/z 521 [M+H]⁺, purity (UV/MS) 95/81.

### 3-acetyl-1-(3-(2,3-dihydro-1H-inden-2-ylamino)propyl)-1H-indole-7-carbonitrile C548

Amount made: 3.0 mg. LCMS m/z 358 [M+H]⁺, purity (UV/MS) 99/80.

### 3-acetyl-1-(3-(2-phenoxyethylamino)propyl) I H-indole-7-carbonitrile C549

Amount made: 3.7 mg. LCMS m/z 362 [M+H]⁺, purity (UV/MS) 96/80.

### 3-acetyl-1-(3-(3-(2-(4-chlorophenyl)-2-oxoethyl)-8-azabicyclo[3.2.1]octan-8-yl)propyl)-1H-indole-7-carbonitrile C550

Amount made: 5.1 mg. LCMS m/z 488 [M+H]⁺, purity (UV/MS) 95/80.

### 3-acetyl-1-(3-(3-(2-chlorobenzyl)piperidin-1-yl)propyl)-1H-indole-7-carbonitrile C551

Amount made: 2.1 mg. LCMS m/z 434 [M+H]⁺, purity (UV/MS) 100/80.

### 3-acetyl-1-(3-(3-(2-chlorophenoxy)-8-azabicyclo[3.2.1]octan-8-yl)propyl)-1H-indole-7-carbonitrile C552

Amount made: 3.3 mg. LCMS m/z 462 [M+H]⁺, purity (UV/MS) 94/100.

### 3-acetyl-1-(3-(3-(4-chlorophenoxy)piperidin-1-yl)propyl)-1H-indole-7-carbonitrile C553

Amount made: 3.0 mg. LCMS m/z 436 [M+H]⁺, purity (UV/MS) 100/100.

### 3-acetyl-1-(3-(3-(4-fluorophenoxy)-8-azabicyclo[3.2.1]octan-8-yl)propyl)-1H-indole-7-carbonitrile C554

Amount made: 3.9 mg. LCMS m/z 446 [M+H]⁺, purity (UV/MS) 100/100.

### 3-acetyl-1-(3-(3-phenethyl-8-azabicyclo[3.2.1]octan-8-yl)propyl)-1H-indole-7-carbonitrile C555

Amount made: 2.8 mg. LCMS m/z 440 [M+H]⁺, purity (UV/MS) 92/90.

### 3-acetyl-1-(3-(4-(2-(4-chloronaphthalen-1-yloxy)ethyl)piperazin-1-yl)propyl)-1H-indole-7-carbonitrile C556

Amount made: 0.4 mg. LCMS m/z 515 [M+H]⁺, purity (UV/MS) 100/70.

### 3-acetyl-1-(3-(4-(2-(4-chlorophenoxy)ethyl)piperazin-1-yl)propyl)-1H-indole-7-carbonitrile C557

Amount made: 7.2 mg. LCMS m/z 465 [M+H]⁺, purity (UV/MS) 100/90.

### 3-acetyl-1-(3-(4-(2-(4-chlorophenoxy)ethyl)piperidin-1-yl)propyl)-1H-indole-7-carbonitrile C558

Amount made: 1.8 mg. LCMS m/z 464 [M+H]⁺, purity (UV/MS) 100/90.

### 3-acetyl-1-(3-(4-(2,4-dichlorobenzyl)piperazin-1-yl)propyl)-1H-indole-7-carbonitrile C559

Amount made: 6.4 mg. LCMS m/z 469 [M+H]⁺, purity (UV/MS) 100/90.

### 3-acetyl-1-(3-(4-(2-chlorobenzyl)-1,4-diazepan-1-yl)propyl)-1H-indole-7-carbonitrile C560

Amount made: 2.9 mg. LCMS m/z 449 [M+H]⁺, purity (UV/MS) 69/60.

### 3-acetyl-1-(3-(4-(2-oxoindolin-1-ylpiperidin-1-yl)propyl)-1H-indole-7-carbonitrile C561

Amount made: 1.5 mg. LCMS m/z 441 [M+H]⁺, purity (UV/MS) 93/80.

### 3-acetyl-1-(3-(4-(2-phenoxyethyl)piperazin-1-yl)propyl)-1H-indole-7-carbonitrile C562

Amount made: 7.1 mg. LCMS m/z 431 [M+H]⁺, purity (UV/MS) 100/90.

### 3-acetyl-1-(3-(4-(3-(pyridin-3-yl)-1,2,4-oxadiazol-5-yl)piperidin-1-yl)propyl)-1H-indole-7-carbonitrile C563

Amount made: 6.6 mg. LCMS m/z 455 [M+H]⁺, purity (UV/MS) 92/80.

### 3-acetyl-1-(3-(4-(3-(pyridin-4-yl)-1,2,4-oxadiazol-5-yl)piperidin-1-yl)propyl)-1H-indole-7-carbonitrile C564

Amount made: 6.6 mg. LCMS m/z 455 [M+H]⁺, purity (UV/MS) 100/90.

### 3-acetyl-1-(3-(4-(3-chloro-5-(trifluoromethyl 1 pyridin-2-yl)piperazin-1-yl)propyl)-1H-indole-7-carbonitrile C565

Amount made: 6.7 mg. LCMS m/z 490 [M+H]⁺, purity (UV/MS) 100/90.

### 3-acetyl-1-(3-(4-(3-chlorophenoxy)piperidin-1-yl)propyl 1H-indole-7-carbonitrile C566

Amount made: 5.6 mg. LCMS m/z 436 [M+H]⁺, purity (UV/MS) 100/100.

### 3-acetyl-1-(3-(4-oxospiro[chroman-2,4′-piperidine]-1-yl)propyl)-1H-indole-7-carbonitrile C567

Amount made: 2.7 mg. LCMS m/z 442 [M+H]⁺, purity (UV/MS) 94/90.

### 3-acetyl-1-(3-(4-phenethyl-1,4-diazepan-1-yl)propyl)-1H-indole-7-carbonitrile C568

Amount made: 4.7 mg. LCMS m/z 429 [M+H]⁺, purity (UV/MS) 80/80.

### 4-(4-(3-(3-acetyl-7-methoxy-1H-indol-1-yl)propyl)piperazin-1-yl)benzonitrile C569

Amount made: 2.4 mg. LCMS m/z 417 [M+H]⁺, purity (UV/MS) 95/80.

### cyclopropyl(1-(3-(2,3-dihydro-1H-inden-2-ylamino)propyl)-7-methoxy-1H-indol-3-yl)methanone C570

Amount made: 4.8 mg. LCMS m/z 389 [M+H]⁺, purity (UV/MS) 97/80.

### cyclopropyl(1-(3-(3-(4-fluorophenoxy)-8-azabicyclo[3.2.1]octan-8-yl)propyl)-7-methoxy-1H-indol-3-yl)methanone C571

Amount made: 2.7 mg. LCMS m/z 477 [M+H]⁺, purity (UV/MS) 96/60.

### cyclopropyl(1-(3-(4-(4-fluorobenzyl-1,4-diazepan-1-yl)propyl)-7-methoxy-1H-indol-3-yl)methanone C572

Amount made: 5.0 mg. LCMS m/z 464 [M+H]⁺, purity (UV/MS) 100/90.

### cyclopropyl(1-(3-(4-(4-fluorophenoxy)piperidin-1-yl)propyl)-7-methoxy-1H-indol-3-yl)methanone C573

Amount made: 5.9 mg. LCMS m/z 451 [M+H]⁺, purity (UV/MS) 95/80.

### cyclopropyl(7-methoxy-1-(3-(2-phenoxyethylamino)propyl)-1H-indol-3-yl)methanone C574

Amount made: 4.2 mg. LCMS m/z 393 [M+H]⁺, purity (UV/MS) 95/80.

### cyclopropyl(7-methoxy-1-(3-(3-pentyl-8-azabicyclo[3.2.1]octan-8-yl)propyl)-1H-indol-3-yl)methanone C575

Amount made: 2.4 mg. LCMS m/z 437 [M+H]⁺, purity (UV/MS) 86/80.

### cyclopropyl(7-methoxy-1-(3-(3-phenethyl-8-azabicyclo[3.2.1]octan-8-yl)propyl)-1H-indol-3-yl)methanone C576

Amount made: 5.4 mg. LCMS m/z 471 [M+H]⁺, purity (UV/MS) 83/60.

### cyclopropyl(7-methoxy-1-(3-(4-(2-methoxyphenyl)piperidin-1-yl)propyl)-1H-indol-3-yl)methanone C577

Amount made: 4.8 mg. LCMS m/z 447 [M+H]⁺, purity (UV/MS) 96/60.

### cyclopropyl(7-methoxy-1-(3-(4-(2-phenoxyethyl)piperazin-1-yl)propyl)-1H-indol-3-yl)methanone C578

Amount made: 7.8 mg. LCMS m/z 462 [M+H]⁺, purity (UV/MS) 98/90.

### cyclopropyl(7-methoxy-1-(3-(4-(3-(pyridin-3-yl)-1,2,4-oxadiazol-5-yl)piperidin-1-yl)propyl)-1H-indol-3-yl)methanone C579

Amount made: 6.0 mg. LCMS m/z 486 [M+H]⁺, purity (UV/MS) 100/90.

### cyclopropyl(7-methoxy-1-(3-(4-(3-(pyridin-4-yl)-1,2,4-oxadiazol-5-yl)piperidin-1-yl)propyl)-1H-indol-3-yl)methanone C580

Amount made: 7.7 mg. LCMS m/z 486 [M+H]⁺, purity (UV/MS) 99/90.

### cyclopropyl(7-methoxy-1-(3-(4-phenethyl-1,4-diazepan-1-yl)propyl)-1H-indol-3-yl)methanone C581

Amount made: 5.8 mg. LCMS m/z 460 [M+H]⁺, purity (UV/MS) 98/90.

### ethyl 1-(3-(3-acetyl-7-methoxy-1H-indol-1-yl)propyl)piperidine-4-carboxylate C582

Amount made: 7.6 mg. LCMS m/z 387 [M+H]⁺, purity (UV/MS) 80/70.

### 1-(1-C2-(3-(4-fluorophenyl)-3-hydroxy-8-azabicyclo[3.2.1]octan-8-yl)ethyl)-1H-indol-3-yl)ethanone C583

Amount made: 2.8 mg. LCMS m/z 407 [M+H]⁺, purity (UV/MS) 98/70.

### 1-(1-(2-(3-(cyclopropylmethoxy)-8-azabicyclo[3.2.1]octan-8-yl)ethyl)-1H-indol-3-yl)ethanone C584

Amount made: 6.0 mg. LCMS m/z 367 [M+H]⁺, purity (UV/MS) 98/77.

### 1-(1-(2-(4-(2-methoxyphenyl)piperidin-1-yl)ethyl)-1H-indol-3-yl)ethanone C585

Amount made: 3.4 mg. LCMS m/z 377 [M+H]⁺, purity (UV/MS) 100/87.

### 1-(2-(4-benzylpiperidin-1-yl)ethyl-1H-indol-3-yl)ethanone C586

Amount made: 1.0 mg. LCMS m/z 361 [M+H]⁺, purity (UV/MS) 100/70.

### 1-(1-(2-(4-butylpiperidin-1-yl)ethyl)-1H-indol-3-yl)ethanone C587

Amount made: 2.8 mg. LCMS m/z 327 [M+H]⁺, purity (UV/MS) 100/91.

### 1-(1-(2-(4-propoxypiperidin-1-yl)ethyl)-1H-indol-3-yl)ethanone C588

Amount made: 1.8 mg. LCMS m/z 329 [M+H]⁺, purity (UV/MS) 100/90.

### 1-(1-(2-hydroxy-3-(4-(1-phenylethyl)piperazin-1-yl)propyl)-1H-indol-3-yl)ethanone C589

Amount made: 11.5 mg. LCMS m/z 406 [M+H]⁺, purity (UV/MS) 100/70.

### 1-(1-(2-hydroxy-3-(4-propoxypiperidin-1-yl)propyl)-1H-indol-3-yl)ethanone C590

Amount made: 5.0 mg. LCMS m/z 359 [M+H]⁺, purity (UV/MS) 90/67.

### 1-(1-(2-methyl-3-(4-(1-phenylethyl)piperazin-1-yl)propyl)-1H-indol-3-yl)ethanone C591

Amount made: 3.7 mg. LCMS m/z 404 [M+H]⁺, purity (UV/MS) 98/55.

### 1-(1-(2-methyl-3-(4-propoxypiperidin-1-yl)propyl)-1H-indol-3-yl)ethanone C592

Amount made: 3.0 mg. LCMS m/z 357 [M+H]⁺, purity (UV/MS) 80/73.

### 1-(1-(3-(3-(2-methoxyethyl)-8-azabicyclo[3.2.1]octan-8-yl)propyl)-1H-indol-3-yl)ethanone C593

Amount made: 6.4 mg. LCMS m/z 369 [M+H]⁺, purity (UV/MS) 96/93.

### 1-(1-(3-(3-(4-chlorophenoxy)-8-azabicyclo[3.2.1]octan-8-yl)propyl)-1H-indol-3-yl)-2,2,2-trifluoroethanone C594

Amount made: 2.8 mg. LCMS m/z 491 [M+H]⁺, purity (UV/MS) 98/64.

### 1-(1-(3-(3-(4-chlorophenoxy)-8-azabicyclo[3.2.1]octan-8-yl)propyl)-1H-indol-3-yl)-3-methylbutan-1-one C595

Amount made: 3.4 mg. LCMS m/z 479 [M+H]⁺, purity (UV/MS) 100/84.

### 1-(1-(3-(3-(4-chlorophenoxy)-8-azabicyclo[3.2.1]octan-8-yl)propyl)-1H-indol-3-yl)ethanone C596

Amount made: 16.0 mg. LCMS m/z 437 [M+H]⁺, purity (UV/MS) 95/72.

### 1-(1-(3-(3-(4-chlorophenoxy)-8-azabicyclo[3.2.1]octan-8-yl)propyl)-1H-indol-3-yl)ethanone C597

Amount made, 16.0 mg. LCMS m/z 437 [M+H]⁺, purity (UV/MS) 97/64.

### 1-(1-(3-(3-(4-chlorophenoxy)-8-azabicyclo[3.2.1]octan-8-yl)propyl)-4-methoxy-1H-indol-3-yl)ethanone C598

Amount made: 2.1 mg, LCMS m/z 467 [M+H]⁺, purity (UV/MS) 79/39.

### 1-(1-(3-(3-(4-chlorophenoxy)-8-azabicyclo[3.2.1]octan-8-yl)propyl)-5-methoxy-1H-indol-3-yl)ethanone C599

Amount made: 2.1 mg. LCMS m/z 467 [M+H]⁺, purity (UV/MS) 87/38.

### 1-(1-C3-(3-(4-chlorophenoxy)-8-azabicyclo[3.2.1]octan-8-yl)propyl)-5-methoxy-1H-indol-3-yl)pentan-1-one C600

Amount made: 3.1 mg. LCMS m/z 509 [M+H]⁺, purity (UV/MS) 77/55.

### 1-(1-(3-(3-(4-chlorophenoxy)-8-azabicyclo[3.2.1]octan-8-yl)propyl)-6-methoxy-1H-indol-3-yl)ethanone C601

Amount made: 3.6 mg. LCMS m/z 467 [M+H]⁺, purity (UV/MS) 93/50.

### 1-(1-(3-(3-(4-fluorophenyl)-3-hydroxy-8-azabicyclo[3.2.1]octan-8-yl)propyl)-1H-indol-3-yl)propan-1-one C602

Amount made: 3.3 mg. LCMS m/z 435 [M+H]⁺, purity (UV/MS) 98/80.

### 1-(1-(3-(3-(cyclopropylmethoxy)-8-azabicyclo[3.2.1]octan-8-yl)-2-methyl)propyl)-1H-indol-3-yl)ethanone C603

Amount made: 9.3 mg. LCMS m/z 395 [M+H]⁺, purity (UV/MS) 100/89.

### 1-(1-(3-(3-(cyclopropylmethoxy)-8-azabicyclo[3.2.1]octan-8-yl)propyl)-1H-indol-3-yl)-2,2,2-trifluoroethanone C604

Amount made: 8.3 mg. LCMS m/z 435 [M+H]⁺, purity (UV/MS) 99/95.

### 1-(1-(3-(3-(cyclopropylmethoxy)-8-azabicyclo[3.2.1]octan-8-yl)propyl)-1H-indol-3-yl)-3-methylbutan-1-one C605

Amount made: 8.7 mg. LCMS m/z 423 [M+H]⁺, purity (UV/MS) 98/91.

### 1-(1-(3-(3-(cyclopropylmethoxy)-8-azabicyclo[3.2.1]octan-8-yl)propyl)-1H-indol-3-yl)ethanone C606

Amount made: 16.3 mg. LCMS m/z 381 [M+H]⁺, purity (UV/MS) 99/88.

### 1-(1-(3-(3-(cyclopropylmethoxy)-8-azabicyclo[3.2.1]octan-8-yl)propyl)-1H-indol-3-yl)propan-1-one C607

Amount made: 5.6 mg. LCMS m/z 395 [M+H]⁺, purity (UV/MS) 100/75.

### 1-(1-(3-(3-(cyclopropylmethoxy)-8-azabicyclo[3.2.1]octan-8-yl)propyl)-4-methoxy-1H-indol-3-yl)ethanone C608

Amount made: 4.7 mg. LCMS m/z 411 [M+H]⁺, purity (UV/MS) 99/81.

### 1-(1-(3-(3-(cyclopropylmethoxy)-8-azabicyclo[3.2.1]octan-8-yl)propyl)-5-methoxy-1H-indol-3-yl)ethanone C609

Amount made: 4.1 mg. LCMS m/z 411 [M+H]⁺, purity (UV/MS) 100/86.

### 1-(1-(3-(3-(cyclopropylmethoxy 8-azabicyclo[3.2.1]octan-8-yl)propyl)-5-methoxy-1H-indol-3-yl)pentan-1-one C610

Amount made: 4.8 mg. LCMS m/z 453 [M+H]⁺, purity (UV/MS) 99/71.

### 1-(1-(3-(3-(cyclopropylmethoxy)-8-azabicyclo[3.2.1]octan-8-yl)propyl)-6-methoxy-1H-indol-3-yl)ethanone C611

Amount made: 10.5 mg. LCMS m/z 411 [M+H]⁺, purity (UV/MS) 100/93.

### 1-(1-(3-(3,4-dihydroisoquinolin-2(1H)-yl)propyl)-1H-indol-3-yl)ethanone C612

Amount made: 12.0 mg. LCMS m/z 333 [M+H]⁺, purity (UV/MS) 88/60.

### 1-(1-(3-(3-butyl-3,8-diazabicyclo[3.2.1]octan-8-yl)propyl)-1H-indol-3-yl)ethanone C613

Amount made: 18.1 mg. LCMS m/z 368 [M+H]⁺, purity (UV/MS) 95/61.

### 1-(1-(3-(3-pentyl-8-azabicyclo[3.2.1]octan-8-yl)propyl)-1H-indol-3-yl)ethanone C614

Amount made: 14.0 mg. LCMS m/z 381 [M+H]⁺, purity (UV/MS) 97/79.

### 1-(1-(3-(3-pentyl-8-azabicyclo[3.2.1]octan-8-yl)propyl)-1H-indol-3-yl)ethanone C615

Amount made: 15.0 mg. LCMS m/z 381 [M+H]⁺, purity (UV/MS) 95/77.

### 1-(1-(3-(3-phenethyl-8-azabicyclo[3.2.1]octan-8-yl)propyl)-1H-indol-3-yl)ethanone C616

Amount made: 9.8 mg. LCMS m/z 415 [M+H]⁺, purity (UV/MS) 99/84.

### 1-(1-(3-(4-((tetrahydrofuran-2-yl)methyl)piperazin-1-yl)propyl)-1H-indol-3-yl)ethanone C617

Amount made: 8.2 mg. LCMS m/z 370 [M+H]⁺, purity (UV/MS) 90/57.

### 1-(1-(3-(4-(1H-indol-4-yl)piperazin-1-yl)propyl)-1H-indol-3-yl)ethanone C618

Amount made: 15.7 mg. LCMS m/z 401 [M+H]⁺, purity (UV/MS) 82/41.

### 1-(1-(3-(4-(1-phenylethyl)piperazin-1-yl)propyl)-1H-indol-3-yl)ethanone C619

Amount made: 10.7 mg. LCMS m/z 390 [M+H]⁺, purity (UV/MS) 93/72.

### 1-(1-(3-(4-(1-phenylethyl)piperazin-1-yl)propyl)-1H-indol-3-yl)propan-1-one C620

Amount made: 10.9 mg. LCMS m/z 404 [M+H]⁺, purity (UV/MS) 100/84.

### 1-(1-(3-(4-(2-(diisopropylamino)ethyl)piperazin-1-yl)propyl)-1H-indol-3-yl)ethanone C621

Amount made: 13.2 mg. LCMS m/z 413 [M+H]⁺, purity (UV/MS) 95/50.

### 1-(1-(3-(4-(2-(methylthio)phenyl)piperazin-1-yl)propyl)-1H-indol-3-yl)ethanone C622

Amount made: 11.3 mg. LCMS m/z 408 [M+H]⁺, purity (UV/MS) 84/51.

### 1-(1-(3-(4-(2,4-difluorobenzoyl)piperidin-1-yl)propyl)-1H-indol-3-yl)ethanone C623

Amount made: 4.4 mg. LCMS m/z 425 [M+H]⁺, purity (UV/MS) 72/53.

### 1-(1-(3-(4-(2-chlorophenoxy)piperidin-1-yl)-2-hydroxypropyl)-1H-indol-3-yl)ethanone C624

Amount made: 17.4 mg. LCMS m/z 427 [M+H]⁺, purity (UV/MS) 100/95.

### 1-(1-(3-(4-(2-chlorophenoxy)piperidin-1-yl)propyl)-1H-indol-3-yl)-2,2,2-trifluoroethanone C625

Amount made: 10.1 mg. LCMS m/z 465 [M+H]⁺, purity (UV/MS) 97/86.

### 1-(1-(3-(4-(2-chlorophenoxy)piperidin-1-yl)propyl)-1H-indol-3-yl)-3-methylbutan-1-one C626

Amount made: 8.7 mg. LCMS m/z 453 [M+H]⁺, purity (UV/MS) 96/80.

### 1-(1-(3-(4-(2-chlorophenoxy)piperidin-1-yl)propyl)-1H-indol-3-yl)ethanone hydrochloride C627

Amount made: 20.2 mg. LCMS m/z 441 [M+H]⁺, purity (UV/MS) 98/72.

### 1-(1-(3-(4-(2-chlorophenoxy)piperidin-1-yl)propyl) I H-indol-3-yl)propan-1-one C628

Amount made: 5.9 mg. LCMS m/z 425 [M+H]⁺, purity (UV/MS) 100/81.

### 1-(1-(3-(4-(2-chlorophenoxy)piperidin-1-yl)propyl)-4-methoxy-1H-indol-3-yl)ethanone C629

Amount made: 4.6 mg. LCMS m/z 441 [M+H]⁺, purity (UV/MS) 99/89.

### 1-(1-(3-(4-(2-chlorophenoxy)piperidin-1-yl)propyl)-5-methoxy-1H-indol-3-yl)ethanone C630

Amount made: 5.6 mg. LCMS m/z 441 [M+H]⁺, purity (UV/MS) 99/86.

### 1-(1-(3-(4-(2-chlorophenoxy)piperidin-1-yl)propyl)-5-methoxy-1H-indol-3-yl)pentan-1-one C631

Amount made: 5.5 mg. LCMS m/z 483 [M+H]⁺, purity (UV/MS) 96/81.

### 1-(1-(3-(4-(2-chlorophenoxy)piperidin-1-yl)propyl)-6-methoxy-1H-indol-3-yl)ethanone C632

Amount made: 12.3 mg. LCMS m/z 441 [M+H]⁺, purity (UV/MS) 98/90.

### 1-(1-(3-(4-(2-chlorophenyl)piperazin-1-yl)propyl)-1H-indol-3-yl)ethanone hydrochloride C633

Amount made: 9.1 mg. LCMS m/z 396 [M+H]⁺, purity (UV/MS) 82/58.

### 1-(1-(3-(4-(2-ethoxyethyl)piperazin-1-yl)propyl)-1H-indol-3-yl)ethanone C634

Amount made: 6.3 mg. LCMS m/z 358 [M+H]⁺, purity (UV/MS) 88/57.

### 1-(1-(3-(4-(2-methoxyethyl)piperazin-1-yl)propyl)-1H-indol-3-yl)ethanone C636

Amount made: 9.1 mg. LCMS m/z 344 [M+H]⁺, purity (UV/MS) 96/86.

### 1-(1-(3-(4-(2-methoxyphenyl)piperidin-1-yl)-2-methyl)propyl)-1H-indol-3-yl)ethanone C637

Amount made: 4.8 mg. LCMS m/z 405 [M+H]⁺, purity (UV/MS) 99/75.

### 1-(1-(3-(4-(2-methoxyphenyl)piperidin-1-yl)propyl)-1H-indol-3-yl)-3-methylbutan-1-one C638

Amount made: 12.0 mg. LCMS m#z 433 [M+H]⁺, purity (UV/MS) 97/72.

### 1-(1-(3-(4-(2-methoxyphenyl)piperidin-1-yl)propyl)-1H-indol-3-yl)ethanone C639

Amount made: 4.8 mg. LCMS m/z 391 [M+H]⁺, purity (UV/MS) 81/69.

### 1-(1-(3-(4-(2-methoxyphenyl)piperidin-1-yl)propyl)-1H-indol-3-yl)propan-1-one C640

Amount made: 10.9 mg. LCMS m/z 405 [M+H]⁺, purity (UV/MS) 97/81.

### 1-(1-(3-(4-(4-chlorophenoxy)piperidin-1-yl)-2-hydroxypropyl)-1H-indol-3-yl)ethanone C641

Amount made: 9.6 mg. LCMS m/z 427 [M+H]⁺, purity (UV/MS) 80/58.

### 1-(1-(3-(4-(4-chloro-henoxy)piperidin-1-yl)propyl)-1H-indol-3-yl)-2,2,2-trifluoroethanone C642

Amount made: 5.2 mg. LCMS m/z 465 [M+H]⁺, purity (UV/MS) 74/75.

### 1-(1-(3-(4-(4-chlorophenoxy)piperidin-1-yl)propyl)-1H-indol-3-yl)-3-methylbutan-1-one C643

Amount made: 4.7 mg. LCMS m/z 453 [M+H]⁺, purity (UV/MS) 92/65.

### 1-(1-(3-(4-(4-chlorophenoxy)piperidin-1-yl)propyl)-1H-indol-3-yl)ethanone C644

Amount made: 16.6 mg. LCMS m/z 411 [M+H]⁺, purity (UV/MS) 96/71.

### 1-(1-(3-(4-(4-chlorophenoxy)piperidin-1-yl)propyl)-4-methoxy-1H-indol-3-yl)ethanone C645

Amount made: 2.3 mg. LCMS m/z 441 [M+H]⁺, purity (UV/MS) 92/51.

### 1-(1-(3-(4-(4-chlorophenoxy)piperidin-1-yl)propyl)-5-methoxy-1H-indol-3-yl)ethanone C646

Amount made: 3.0 mg. LCMS m/z 441 [M+H]⁺, purity (UV/MS) 92/57.

### 1-(1-(3-(4-(4-chlorophenoxy)piperidin-1-yl)propyl)-5-methoxy-1H-indol-3-yl)pentan-1-one C647

Amount made: 3.5 mg. LCMS m/z 483 [M+H]⁺, purity (UV/MS) 84/47.

### 1-(1-(3-(4-(4-chlorophenoxy)piperidin-1-yl)propyl)-6-methoxy-1H-indol-3-yl)ethanone C648

Amount made: 4.8 mg. LCMS m/z 441 [M+H]⁺, purity (UV/MS) 85/58.

### 1-(1-(3-(4-(4-chlorophenylsulfonyl)piperidin-1-yl)propyl)-1H-indol-3-yl)-2,2,2-trifluoroethanone C649

Amount made: 5.6 mg. LCMS m/z 513 [M+H]⁺, purity (UV/MS) 100/70.

### 1-(1-(3-(4-(4-chlorophenylsulfonyl)piperidin-1-yl)propyl)-1H-indol-3-yl)ethanone hydrochloride C650

Amount made: 21.0 mg. LCMS m/z 459 [M+H]⁺, purity (UV/MS) 97/62.

### 1-(1-(3-(4-(4-chlorophenylsulfonyl)piperidin-1-yl)propyl)-1H-indol-3-yl)propan-1-one C651

Amount made: 10.6 mg. LCMS m/z 473 [M+H]⁺, purity (UV/MS) 100/80.

### 1-(1-(1-(3-(4-(4-chlorophenylthio)piperidin-1-yl)propyl)-1H-indol-3-yl)ethanone hydrochloride C652

Amount made: 14.4 mg. LCMS m/z 427 [M+H]⁺, purity (UV/MS) 98/72.

### 1-(1-(3-(4-(4-fluorophenoxy)piperidin-1-yl)-2-hydroxypropyl)-1H-indol-3-yl)ethanone C653

Amount made: 9.6 mg. LCMS m/z 411 [M+H]⁺, purity (UV/MS) 100/87.

### 1-(1-(3-(4-(4-fluoro-henoxy)piperidin-1-yl)-2-methyl)propyl)-1H-indol-3-yl)ethanone C654

Amount made: 1.1 mg. LCMS m/z 409 [M+H]⁺, purity (UV/MS) 100/78.

### 1-(1-(3-(4-(4-fluorophenoxy)-piperidin-1-yl)propyl)-1H-indol-3-yl)-3-methylbutan-1-one C655

Amount made: 10.2 mg. LCMS m/z 437 [M+H]⁺, purity (UV/MS) 98/80.

### 1-(1-(3-(4-(4-fluorophenoxy)piperidin-1-yl)propyl)-1H-indol-3-yl)ethanone hydrochloride C656

Amount made: 9.0 mg. LCMS m/z 395 [M+H]⁺, purity (UV/MS) 88/76.

### 1-(1-(3-(4-(4-fluorophenoxy)piperidin-1-yl)propyl)-1H-indol-3-yl)propan-1-one C657

Amount made: 9.8 mg. LCMS m/z 409 [M+H]⁺, purity (UV/MS) 99/80.

### 1-(1-(3 (4-(4-fluorophenoxy)piperidin-1-yl)propyl)-4-methoxy-1H-indol-3-yl)ethanone C658

Amount made: 4.5 mg. LCMS m/z 425 [M+H]⁺, purity (UV/MS) 98/86.

### 1-(3-(4-(4-fluorophenoxy)piperidin-1-yl)propyl)-5-methoxy-1H-indol-3-yl)ethanone C659

Amount made: 5.2 mg. LCMS m/z 425 [M+H]⁺, purity (UV/MS) 100/89.

### 1-(1-(3-(4-(4-fluorophenoxy)piperidin-1-yl)propyl)-5-methoxy-1H-indol-3-yl)pentan-1-one C660

Amount made: 5.5 mg. LCMS m/z 467 [M+H]⁺, purity (UV/MS) 98/80.

### 1-(1-(3-(4-(4-fluorophenoxy)piperidin-1-yl)propyl)-6-methoxy-1H-indol-3-yl)ethanone C661

Amount made: 10.7 mg LCMS m/z 425 [M+H]⁺, purity (UV/MS) 100/96.

### 1-(1-(3-(4-(allyloxy)piperidin-1-yl)propyl)-1H-indol-3-yl)ethanone C662

Amount made: 9.0 mg. LCMS m/z 341 [M+H]⁺, purity (UV/MS) 99/70.

### 1-(1-(3-(4-(benzo[d]thiazol-2-yl)piperidin-1-yl)-2-hydroxypropyl)-1H-indol-3-yl)ethanone C663

Amount made: 5.6 mg. LCMS m/z 434 [M+H]⁺, purity (UV/MS) 97/79.

### 1-(1-(3-(4-(benzo[d]thiazol-2-yl)piperidin-1-yl)-2-methyl)propyl)-1H-indol-3-yl)ethanone C664

Amount made: 1.0 mg. LCMS m/z 432 [M+H]⁺, purity (UV/MS) 91/58.

### 1-(1-(3-(4-(benzo[d]thiazol-2-yl)piperidin-1-yl)propyl)-1H-indol-3-yl)-2,2,2-trifluoroethanone C665

Amount made: 8.4 mg. LCMS m/z 472 [M+H]⁺, purity (UV/MS) 100/98.

### 1-(1-(3-(4-(benzo[d]thiazol-2-ylpiperidin-1-yl)propyl)-1H-indol-3-yl)-3-methylbutan-1-one C666

Amount made: 10.4 mg. LCMS m/z 460 [M+H]⁺, purity (UV/MS) 100/94.

### 1-(1-(3-(4-(benzo[d]thiazol-2-yl)piperidin-1-yl)propyl)-1H-indol-3-yl)ethanone C667

Amount made: 20.7 mg. LCMS m/z 418 [M+H]⁺, purity (UV/MS) 99/68.

### 1-(1-(3-(4-(benzo[d]thiazol-2-yl)piperidin-1-yl)propyl)-1H-indol-3-yl)propan-1-one C668

Amount made: 9.4 mg. LCMS m/z 432 [M+H]⁺, purity (UV/MS) 97/82.

### 1-(1-(3-(4-(benzo[d]thiazol-2-yl)piperidin-1-yl)propyl)-4-methoxy-1H-indol-3-yl)ethanone C669

Amount made: 1.7 mg. LCMS m/z 448 [M+H]⁺, purity (UV/MS) 93/72.

### 1-(1-(3-(4-(benzo[d]thiazol-2-yl)piperidin-1-yl)propyl)-6-methoxy-1H-indol-3-yl)ethanone C670

Amount made: 11.0 mg. LCMS m/z 448 [M+H]⁺, purity (UV/MS) 99/86.

### 1-(1-(3-(4-(cyclopropylmethoxy)piperidin-1-yl)propyl)-1H-indol-3-yl)ethanone C671

Amount made: 9.3 mg. LCMS m/z 355 [M+H]⁺, purity (UV/MS) 97/81.

### 1-(1-(3-(4-(methoxymethyl)piperidin-1-yl)propyl)-1H-indol-3-yl)ethanone C672

Amount made: 14.2 mg. LCMS m/z 329 [M+H]⁺, purity (UV/MS) 100/87.

### 1-(1 (3-(4-(pyrrolidin-1-yl)piperidin-1-yl)propyl)-1H-indol-3-yl)ethanone C673

Amount made: 7.1 mg. LCMS m/z 354 [M+H]⁺, purity (UV/MS) 96/83.

### 1-(1-(3-(4-benzoylpiperidin-1-yl)-2-hydroxypropyl)-1H-indol-3-yl)ethanone C674

Amount made: 14.3 mg. LCMS m/z 405 [M+H]⁺, purity (UV/MS) 99/95.

### 1-(1-(3-(4-benzoylpiperidin-1-yl)-2-methyl)propyl)-1H-indol-3-yl)ethanone C675

Amount made: 1.9 mg. LCMS m/z 403 [M+H]⁺, purity (UV/MS) 96/65.

### 1-(1-(3-(4-benzoylpiperidin-1-yl)propyl)-1H-indol-3-yl)-2,2,2-trifluoroethanone C676

Amount made: 8.5 mg. LCMS m/z 443 [M+H]⁺, purity (UV/MS) 100/97.

### 1-(1-(3-(4-benzoylpiperidin-1-yl)propyl)-1H-indol-3-yl)-3-methylbutan-1-one C677

Amount made: 11.3 mg. LCMS m/z 431 [M+H]⁺, purity (UV/MS) 100/89.

### 1-(1-(3-(4-benzoylpiperidin-1-yl)propyl)-1H-indol-3-yl)propan-1-one C678

Amount made: 7.2 mg. LCMS m/z 403 [M+H]⁺, purity (UV/MS) 99/83.

### 1-(1-(3-(4-benzoylpiperidin-1-yl)propyl)-4-methoxy-1H-indol-3-yl)ethanone C679

Amount made: 4.3 mg. LCMS m/z 419 [M+H]⁺, purity (UV/MS) 94/75.

### 1-(1-(3-(4-benzoylpiperidin-1-yl)propyl)-5-bromo-1H-indol-3-yl)ethanone C680

Amount made: 6.4 mg. LCMS m/z 467 [M+H]⁺, purity (UV/MS) 98/76.

### 1-(1-(3-(4-benzoylpiperidin-1-yl)propyl)-5-methoxy-1H-indol-3-yl)ethanone C681

Amount made: 2.7 mg. LCMS m/z 419 [M+H]⁺, purity (UV/MS) 98/64.

### 1-(1-(3-(4-benzoylpiperidin-1-yl)propyl)-5-methoxy-1H-indol-3-yl)pentan-1-one C682

Amount made: 5.1 mg. LCMS m/z 461 [M+H]⁺, purity (UV/MS) 99/81.

### 1-(1-(3-(4-benzoylpiperidin-1-yl)propyl)-6-bromo-1H-indol-3-yl)ethanone C683

Amount made: 7.8 mg. LCMS m/z 467 [M+H]⁺, purity (UV/MS) 99/85.

### 1-(1-(3-(4-benzoylpiperidin-1-yl)propyl)-6-methoxy-1H-indol-3-yl)ethanone C684

Amount made: 11.7 mg. LCMS m/z 419 [M+H]⁺, purity (UV/MS) 99/87.

### 1-(1-(3-(4-benzyl-4-hydroxypiperidin-1-yl)-2-methyl)propyl)-1H-indol-3-yl)ethanone C685

Amount made: 2.5 mg. LCMS m/z 405 [M+H]⁺, purity (UV/MS) 100/90.

### 1-(1-(3-(4-benzyl-4-hydroxypiperidin-1-yl)propyl)-1H-indol-3-yl)-2,2,2-trifluoroethanone C686

Amount made: 1.2 mg. LCMS m/z 445 [M+H]⁺, purity (UV/MS) 100/100.

### 1-(1-(3-(4-benzyl-4-hydroxypiperidin-1-yl)propyl)-1H-indol-3-yl)propan-1-one C687

Amount made: 5.2 mg. LCMS m/z 405 [M+H]⁺, purity (UV/MS) 99/90.

### 1-(1-(3-(4-benzylpiperidin-1-yl)-2-methyl)propyl)-1H-indol-3-yl)ethanone C688

Amount made: 3.2 mg. LCMS m/z 389 [M+H]⁺, purity (UV/MS) 98/80.

### 1-(1-(3-(4-benzylpiperidin-1-yl)propyl)-1H-indol-3-yl)-2,2,2-trifluoroethanone C689

Amount made: 2.4 mg. LCMS m/z 429 [M+H]⁺, purity (UV/MS) 100/80.

### 1-(1-(3-(4-benzylpiperidin-1-yl)propyl)-1H-indol-3-ethanone C690

Amount made: 12.6 mg. LCMS m/z 375 [M+H]⁺, purity (UV/MS) 99/93.

### 1-(1-(3-(4-benzylpiperidin-1-yl)propyl)-1H-indol-3-yl)propan-1-one C691

Amount made: 6.0 mg. LCMS m/z 389 [M+H]⁺, purity (UV/MS) 100/100.

### 1-(1-(3-(4-benzylpiperidin-1-yl)-2-methyl)propyl)-1H-indol-3-yl)ethanone C692

Amount made: 4.6 mg. LCMS m/z 355 [M+H]⁺, purity (UV/MS) 100/81.

### 1-(1-(3-(4-butylpiperidin-1-yl)propyl)-1H-indol-3-yl)-2,2,2-trifluoroethanone C693

Amount made: 7.2 mg. LCMS m/z 395 [M+H]⁺, purity (UV/MS) 100/100.

### 1-(1-(3-(4-butylpiperidin-1-yl)propyl)-1H-indol-3-yl)-3-methylbutan-1-one C694

Amount made: 9.8 mg. LCMS m/z 383 [M+H]⁺, purity (UV/MS) 99/95.

### 1-(1-(3-(4-butylpiperidin-1-yl)propyl)-1H-indol-3-yl)propan-1-one C695

Amount made: 11.1 mg. LCMS m/z 355 [M+H]⁺, purity (UV/MS) 100/96.

### 1-(1-(3-(4-butylpiperidin-1-yl)propyl)-4-methoxy-1H-indol-3-yl)ethanone C696

Amount made: 4.6 mg. LCMS m/z 371 [M+H]⁺, purity (UV/MS) 100/94.

### 1-(1-(3-(4-butylpiperidin-1-yl)propyl)-5-methoxy-1H-indol-3-yl)ethanone C697

Amount made: 5.2 mg. LCMS m/z 371 [M+H]⁺, purity (UV/MS) 100/96.

### 1-(1-(3-(4-butylpiperidin-1-yl)propyl)-5-methoxy-1H-indol-3-yl)pentan-1-one C698

Amount made: 4.7 mg. LCMS m/z 413 [M+H]⁺, purity (UV/MS) 100/95.

### 1-(1-(3-(4-butylpiperidin-1-yl)propyl)-6-methoxy-1H-indol-3-yl)ethanone C699

Amount made: 9.1 mg. LCMS m/z 371 [M+H]⁺, purity (UV/MS) 100/98.

### 1-(1-(3-(4-hexylidenepiperidin-1-yl)propyl)-1H-indol-3-yl)ethanone C700

Amount made: 8.2 mg. LCMS m/z 367 [M+H]⁺, purity (UV/MS) 79/78.

### 1-(1-(3-(4-hexylpiperidin-1-yl)propyl)-1H-indol-3-yl)ethanone C701

Amount made: 7.7 mg. LCMS m/z 369 [M+H]⁺, purity (UV/MS) 89/80.

### 1-(1-(3-(4-isopentylpiperidin-1-yl)propyl)-1H-indol-3-yl)ethanone C702

Amount made: 3.0 mg. LCMS m/z 355 [M+H]⁺, purity (UV/MS) 87/90.

### 1-(1-(3-(4-pentylidenepiperidin-1-yl)propyl)-1H-indol-3-yl)ethanone C703

Amount made: 17.5 mg. LCMS m/z 353 [M+H]⁺, purity (UV/MS) 94/74.

### 1-(1-(3-(4-phenylpiperazin-1-yl)propyl)-1H-indol-3-yl)ethanone C704

Amount made: 10.7 mg. LCMS m/z 362 [M+H]⁺, purity (UV/MS) 93/55.

### 1-(1-(3-(4-phenylpiperidin-1-yl)propyl)-1H-indol-3-yl)ethanone C705

Amount made: 12.0 mg. LCMS m/z 361 [M+H]⁺, purity (UV/MS) 96/68.

### 1-(1-(3-(4-propoxypiperidin-1-yl)propyl)-1H-indol-3-yl)ethanone C706

Amount made: 10.4 mg. LCMS m/z 343 [M+H]⁺, purity (UV/MS) 99/91.

### 1-(1-(3-(4-propylpiperazin-1-yl)propyl)-1H-indol-3-yl)ethanone C707

Amount made: 9.4 mg. LCMS m/z 328 [M+H]⁺, purity (UV/MS) 78/65.

### 1-(1-(3-(5-butyl-2,5-diazabicyclo[2.2.1]heptan-2-yl)propyl)-1H-indol-3-yl)ethanone C708

Amount made: 3.6 mg. LCMS m/z 354 [M+H]⁺, purity (UV/MS) 88/67.

### 1-(1-(3-(6-methoxy-3,4-dihydro-1H-pyrido[3,4-b]indol-2(9H)-yl)propyl)-1H-indol-3-yl)-3-methylbutan-1-one C709

Amount made: 5.5 mg. LCMS m/z 444 [M+H]⁺, purity (UV/MS) 88/47.

### 1-(1-(3-(methyl(2-(pyridin-2-yl)ethyl)amino)propyl)-1H-indol-3-yl)ethanone C710

Amount made: 10.4 mg. LCMS m/z 336 [M+H]⁺, purity (UV/MS) 85/55.

### 1-(1-(3-(octahydroisoquinolin-2(1H)-yl)propyl)-1H-indol-3-yl)ethanone C711

Amount made: 23.5 mg. LCMS m/z 339 [M+H]⁺, purity (UV/MS) 100/74.

### 1-(1-(3-(piperidin-1-yl)propyl)-1H-indol-3-yl)ethanone C712

Amount made: 14.3 mg. LCMS m/z 285 [M+H]⁺, purity (UV/MS) 100/77.

### 1-(1-(3-morpholinopropyl)-1H-indol-3-yl)ethanone C713

Amount made: 9.4 mg. LCMS m/z 287 [M+H]⁺, purity (UV/MS) 99/77.

### 1-(1-(4-(3-(4-chlorophenoxy)-8-azabicyclo[3.2.1]octan-8-yl)butyl)-1H-indol-3-yl)ethanone C714

Amount made: 3.4 mg. LCMS m/z 451 [M+H]⁺, purity (UV/MS) 68/40.

### 1-(1-(4-(3-(4-fluorophenyl)-3-hydroxy-8-azabicyclo[3.2.1]octan-8-yl)butyl)-1H-indol-3-yl)ethanone C715

Amount made: 2.8 mg. LCMS m/z 435 [M+H]⁺, purity (UV/MS) 98/80.

### 1-(1-(4-(3-(cyclopropylmethoxy)-8-azabicyclo[3.2.1]octan-8-yl)butyl)-1H-indol-3-yl)ethanone C716

Amount made: 7.0 mg. LCMS m/z 395 [M+H]⁺, purity (UV/MS) 100/80.

### 1-(1-(4-(4-(1-phenylethyl)piperazin-1-yl)butyl-1H-indol-3-yl)ethanone C717

Amount made: 11.1 mg. LCMS m/z 404 [M+H]⁺, purity (UV/MS) 98/65.

### 1-(1-(4-(4-(2-chlorophenoxy)piperidin-1-yl)butyl)-1H-indol-3-yl)ethanone C718

Amount made: 11.5 mg. LCMS m/z 425 [M+H]⁺, purity (UV/MS) 100/84.

### 1-(1-(4-(4-(2-methoxyphenyl)piperidin-1-yl)butyl 1H-indol-3-yl)ethanone C719

Amount made: 13.8 mg. LCMS m/z 405 [M+H]⁺, purity (UV/MS) 100/98.

### 1-(1-(4-(4-(4-fluorophenoxy)piperidin-1-yl)butyl 1H-indol-3-yl)ethanone C720

Amount made: 11.9 mg. LCMS m/z 409 [M+H]⁺, purity (UV/MS) 100/87.

### 1-(1-(4-(4-(benzo[d]thiazol-2-yl)piperidin-1-yl)butyl)-1H-indol-3-yl)ethanone C721

Amount made: 11.8 mg. LCMS m/z 432 [M+H]⁺, purity (UV/MS) 98/63.

### 1-(1-(4-(4-benzoylpiperidin-1-yl)butyl)-1H-indol-3-yl)ethanone C722

Amount made: 14.4 mg. LCMS m/z 403 [M+H]⁺, purity (UV/MS) 98/84.

### 1-(1-(4-(4-benzyl-4-hydroxypiperidin-1-yl)butyl)-1H-indol-3-yl)ethanone C723

Amount made: 3.4 mg. LCMS m/z 405 [M+H]⁺, purity (UV/MS) 99/90.

### 1-(1-(4-(4-benzylpiperidin-1-yl)butyl)-1H-indol-3-yl)ethanone C724

Amount made: 6.0 mg. LCMS m/z 389 [M+H]⁺, purity (UV/MS) 99/90.

### 1-(1-(4-(4-butylpiperidin-1-yl)butyl)-1H-indol-3-yl)ethanone C725

Amount made: 13.8 mg. LCMS m/z 355 [M+H]⁺, purity (UV/MS) 100/88.

### 1-(1-(4-(6-methoxy-3,4-dihydro-1H-pyrido[3,4-b]indol-2(9H)-yl)butyl)-1H-indol-3-yl)ethanone C726

Amount made: 3.5 mg. LCMS m/z 416 [M+H]⁺, purity (UV/MS) 92/53.

### 1-(3-(3-acetyl-1H-indol-1-yl)propyl)-N,N-diethylpiperidine-3-carboxamide C727

Amount made: 11.7 mg. LCMS m/z 384 [M+H]⁺, purity (UV/MS) 100/86.

### 1-(4-methoxy-1-(3-(4-(1-phenylethyl)piperazin-1-yl)propyl)-1H-indol-3-yl)ethanone C728

Amount made: 9.5 mg. LCMS m/z 420 [M+H]⁺, purity (UV/MS) 94/66.

### 1-(4-methoxy-1-(3-(4-(2-methoxyphenyl)piperidin-1-yl)propyl)-1H-indol-3-yl)ethanone C729

Amount made: 8.3 mg. LCMS m/z 421 [M+H]⁺, purity (UV/MS) 99/82.

### 1-(4-methoxy-1-(3-(4-propoxypiperidin-1-yl)propyl)-1H-indol-3-yl)ethanone C730

Amount made: 1.6 mg. LCMS m/z 373 [M+H]⁺, purity (UV/MS) 66/66.

### 1-(4-methoxy-1-(3-(6-methoxy-3,4-dihydro-1H-pyrido[3,4-b]indol-2(9H)-yl)propyl)-1H-indol-3-yl)ethanone C731

Amount made: 1.9 mg. LCMS m/z 432 [M+H]⁺, purity (UV/MS) 86/42.

### 1-(5-bromo-1-(3-(3-(cyclopropylmethoxy)-8-azabicyclo[3.2.1]octan-8-yl)propyl)-1H-indol-3-yl)ethanone C732

Amount made: 6.9 mg. LCMS m/z 459 [M+H]⁺, purity (UV/MS) 98/82.

### 1-(5-bromo-1-(3-(4-(1-phenylethyl)piperazin-1-yl)propyl)-1H-indol-3-yl)ethanone C733

Amount made: 9.8 mg. LCMS m/z 468 [M+H]⁺, purity (UV/MS) 99/66.

### 1-(5-bromo-1-(3-(4-(2-chlorophenoxy)piperidin-1-yl)propyl)-1H-indol-3-yl)ethanone C734

Amount made: 5.4 mg. LCMS m/z 489 [M+H]⁺, purity (UV/MS) 99/84.

### 1-(5-bromo-1-(3-(4-(4-fluorophenoxy)piperidin-1-yl)propyl)-1H-indol-3-yl)ethanone C735

Amount made: 6.7 mg. LCMS m/z 473 [M+H]⁺, purity (UV/MS) 97/90.

### 1-(5-bromo-1-(3-(4-propoxypiperidin-1-yl)propyl)-1H-indol-3-yl)ethanone C736

Amount made: 1.7 mg. LCMS m/z 421 [M+H]⁺, purity (UV/MS) 83/71.

### 1-(5-methoxy-1-(3-(4-(1-phenylethyl piperazin-1-yl)propyl)-1H-indol-3-yl)ethanone C737

Amount made: 8.9 mg. LCMS m/z 420 [M+H]⁺, purity (UV/MS) 98/64.

### 1-(5-methoxy-1-(3-(4-(1-phenylethyl)piperazin-1-yl)propyl)-1H-indol-3-yl)pentan-1-one C738

Amount made: 8.6 mg. LCMS m/z 462 [M+H]⁺, purity (UV/MS) 98/86.

### 1-(5-methoxy-1-(3-(4-(2-methoxyphenyl)piperidin-1-yl)propyl)-1H-indol-3-yl)ethanone C739

Amount made: 8.1 mg. LCMS m/z 421 [M+H]⁺, purity (UV/MS) 99/82.

### 1-(5-methoxy-1-(3-(4-(2-methoxyphenyl)piperidin-1-yl)propyl)-1H-indol-3-yl)pentan-1-one C740

Amount made: 7.7 mg. LCMS m/z 463 [M+H]⁺, purity (UV/MS) 97/74.

### 1-(5-methoxy-1-(3-(4-propoxypiperidin-1-yl)propyl)-1H-indol-3-yl)pentan-1-one C741

Amount made: 1.8 mg. LCMS m/z 415 [M+H]⁺, purity (UV/MS) 85/70.

### 1-(5-methoxy-1-(3-(6-methoxy-3,4-dihydro-1H-pyrido[3,4-b]indol-2(9H)-yl)propyl)-1H-indol-3-yl)pentan-1-one C742

Amount made: 3.2 mg. LCMS m/z 474 [M+H]⁺, purity (UV/MS) 81/25.

### 1-(6-bromo-1-(3-(3-(4-chlorophenoxy)-8-azabicyclo[3.2.1]octan-8-yl)propyl)-1H-indol-3-yl)ethanone C743

Amount made: 1.1 mg. LCMS m/z 515 [M+H]⁺, purity (UV/MS) 100/55.

### 1-(6-bromo-1-(3-(3-(cyclopropylmethoxy)-8-azabicyclo[3.2.1]octan-8-yl)propyl)-1H-indol-3-yl)ethanone C744

Amount made: 8.2 mg. LCMS m/z 459 [M+H]⁺, purity (UV/MS) 96/70.

### 1-(6-bromo-1-(3-(4-(1-phenylethyl)piperazin-1-yl)propyl)-1H-indol-3-yl)ethanone C745

Amount made: 9.0 mg. LCMS m/z 468 [M+H]⁺, purity (UV/MS) 100/64.

### 1-(6-bromo-1-(3-(4-(2-chlorophenoxy)piperidin-1-yl)propyl)-1H-indol-3-yl)ethanone C746

Amount made: 8.8 mg. LCMS m/z 489 [M+H]⁺, purity (UV/MS) 97/84.

### 1-(6-bromo-1-(3-(4-(4-chlorophenoxy)piperidin-1-yl)propyl)-1H-indol-3-yl)ethanone C747

Amount made: 2.3 mg. LCMS m/z 489 [M+H]⁺, purity (UV/MS) 100/75.

### 1-(6-bromo-1-(3-(4-(4-fluorophenoxy)piperidin-1-yl)propyl)-1H-indol-3-yl)ethanone C748

Amount made: 5.0 mg. LCMS m/z 473 [M+H]⁺, purity (UV/MS) 95/68.

### 1-(6-bromo-1-(3-(4-butylpiperidin-1-yl)propyl)-1H-indol-3-yl)ethanone C749

Amount made: 4.1 mg. LCMS m/z 419 [M+H]⁺, purity (UV/MS) 100/90.

### 1-(6-bromo-1-(3-(4-propoxypiperidin-1-yl)propyl)-1H-indol-3-yl)ethanone C750

Amount made: 3.2 mg. LCMS m/z 421 [M+1H]⁺, purity (UV/MS) 100/63.

### 1-(6-methoxy-1-(3-(4-(1-phenylethyl)piperazin-1-yl)propyl)-1H-indol-3-yl)ethanone C751

Amount made: 16.1 mg. LCMS m/z 420 [M+H]⁺, purity (UV/MS) 100/91.

### 1-(6-methoxy-1-(3-(4-(2-methoxyphenyl)piperidin-1-yl)propyl)-1H-indol-3-yl)ethanone C752

Amount made: 15.7 mg. LCMS m/z 421 [M+H]⁺, purity (UV/MS) 99/90.

### 1-(6-methoxy-1-(3-(4-propoxypiperidin-1-yl)propyl)-1H-indol-3-yl)ethanone C753

Amount made: 3.0 mg. LCMS m/z 373 [M+H]⁺, purity (UV/MS) 80/68.

### 1-(6-methoxy-1-(3-(6-methoxy-3,4-dihydro-1H-pyrido[3,4-b]indol-2(9H)-yl)propyl)-1H-indol-3-yl)ethanone C754

Amount made: 5.9 mg. LCMS m/z 432 [M+H]⁺, purity (UV/MS) 90/61.

### 2-(4-(3-(3-acetyl-1H-indol-1-yl)propyl)piperazin-1-yl)-1-morpholinoethanone C755

Amount made: 10.2 mg. LCMS m/z 413 [M+H]⁺, purity (UV/MS) 93/70.

### 2-(4-(3-(3-acetyl-1H-indol-1-yl)propyl)piperazin-1-yl)-N,N-dimethylacetamide C756

Amount made: 12.2 mg. LCMS m/z 371 [M+H]⁺, purity (UV/MS) 93/61.

### 2,2,2-trifluoro-1-(1-(3-(3-(4-fluorophenyl)-3-hydroxy-8-azabicyclo[3.2.1]octan-8-yl)propyl)-1H-indol-3-yl)ethanone C757

Amount made: 9.4 mg. LCMS m/z 475 [M+H]⁺, purity (UV/MS) 99/80.

### 2,2,2-trifluoro-1-(1-(3-(4-(1-phenylethyl)piperazin-1-yl)propyl)-1H-indol-3-yl)ethanone C758

Amount made: 9.3 mg. LCMS m/z 444 [M+H]⁺, purity (UV/MS) 99/75.

### 2,2,2-trifluoro-1-(1-(3-(4-(2-methoxyphenyl)piperidin-1-yl)propyl)-1H-indol-3-yl)ethanone C759

Amount made: 10.9 mg. LCMS m/z 445 [M+H]⁺, purity (UV/MS) 99/84.

### 2,2,2-trifluoro-1 (1-(3-(4-(4-fluorophenoxy)piperidin-1-yl)propyl)-1H-indol-3-yl)ethanone C760

Amount made: 9.4 mg. LCMS m/z 449 [M+H]⁺, purity (UV/MS) 99/97.

### 2,2,2-trifluoro-1-(1-(3-(4-propoxypiperidin-1-yl)propyl)-1H-indol-3-yl)ethanone C761

Amount made: 3.0 mg. LCMS m/z 397 [M+H]⁺, purity (UV/MS) 97/85.

### 2,2,2-trifluoro-1-(1-(3-(6-methoxy-3,4-dihydro-1H-pyrido[3,4-b]indol-2(9H)-yl)propyl)-1H-indol-3-yl)ethanone C762

Amount made: 3.0 mg. LCMS m/z 456 [M+H]⁺, purity (UV/MS) 87/44.

### 3-methyl-1-(1-(3-(4-(1-phenylethyl)piperazin-1-yl)propyl)-1H-indol-3-yl)butan-1-one C763

Amount made: 13.3 mg. LCMS m/z 432 [M+H]⁺, purity (UV/MS) 99/91.

### 3-methyl-1-(1-(3-(4-propoxypiperidin-1-yl)propyl)-1H-indol-3-yl)butan-1-one C764

Amount made: 2.3 mg. LCMS m/z 385 [M+H]⁺, purity (UV/MS) 97/79.

### 4-(4-(3-(3-acetyl-1H-indol-1-yl)propyl)piperazin 1-yl)benzonitrile hydrochloride C765

Amount made: 3.7 mg. LCMS m/z 387 [M+H]⁺, purity (UV/MS) 84/41.

### ethyl 1-(2-(3-acetyl-1H-indol-1-yl)ethyl)piperidine-4-carboxylate C766

Amount made: 0.8 mg. LCMS m/z 343 [M+H]⁺, purity (UV/MS) 100/100.

### ethyl 1-(3-(3-(2,2,2-trifluoroacetyl)-1H-indol-1-yl)propyl)piperidine-4-carboxylate C767

Amount made: 5.1 mg. LCMS m/z 411 [M+H]⁺, purity (UV/MS) 93/90.

### ethyl 1-(3-(3-propionyl-1H-indol-1-yl)propyl)piperidine-4-carboxylate C768

Amount made: 1.7 mg. LCMS m/z 371 [M+H]⁺, purity (UV/MS) 92/90.

### N-(1-(3-(3-acetyl-1H-indol-1-yl)propyl)pyrrolidin-3-yl)acetamide C769

Amount made: 12.4 mg. LCMS m/z 328 [M+H]⁺, purity (UV/MS) 98/80,

### 4-(1-(3-(3-(cyclopropylmethoxy)-8-azabicyclo[3.2.1]octan-8-yl)propyl)-1H-indol-3-yl)butan-2-one C770

Amount made: 5.1 mg. LCMS m/z 409 [M+H]⁺, purity (UV/MS) 96/49.

### 4-(1-(3-(4-(1-phenylethyl)piperazin-1-yl)propyl)-1H-indol-3-yl)butan-2-one C771

Amount made: 7.8 mg. LCMS m/z 418 [M+H]⁺, purity (UV/MS) 95/65.

### 4-(1-(3-(4-(2-chlorophenoxy)piperidin-1-yl)propyl)-1H-indol-3-yl)butan-2-one C772

Amount made: 9.1 mg. LCMS m/z 439 [M+H]⁺, purity (UV/MS) 86/50.

### 4-(1-(3-(4-(2-methoxyphenyl)piperidin-1-yl)propyl)-1H-indol-3-yl)butan-2-one C773

Amount made: 10.3 mg. LCMS m/z 419 [M+H]⁺, purity (UV/MS) 80/54.

### 4-(1-(3-(4-(4-fluorophenoxy)piperidin-1-yl)propyl)-1H-indol-3-yl)butan-2-one C774

Amount made: 11.0 mg. LCMS m/z 423 [M+H]⁺, purity (UV/MS) 87/56.

### 4-(1-(3-(4-(benzo[d]thiazol-2-yl)piperidin-1-yl)propyl)-1H-indol-3-yl)butan-2-one C775

Amount made: 9.1 mg. LCMS m/z 446 [M+H]⁺, purity (UV/MS) 88/44.

### 4-(1-(3-(4-benzoylpiperidin-1-yl)propyl)-1H-indol-3-yl)butan-2-one C776

Amount made: 10.2 mg. LCMS m/z 417 [M+H]⁺, purity (UV/MS) 92/58.

### 4-(1-(3-(4-butylpiperidin-1-yl)propyl)-1H-indol-3-yl)butan-2-one C777

Amount made: 10.2 mg. LCMS m/z 369 [M+H]⁺, purity (UV/MS) 83/55.

### 4-(1-(3-(4-propoxypiperidin-1-yl)propyl)-1H-indol-3-yl)butan-2-one C778

Amount made. 2.7 mg. LCMS m/z 371 [M+H]⁺, purity (UV/MS) 70/39.

### methyl 1-(3-((2-(C H-indol-3-yl)ethyl)(methyl)amino)propyl)-1H-indole-3-carboxylate C779

Amount made: 17.7 mg. LCMS m/z 390 [M+H]⁺, purity (UV/MS) 83/52.

### methyl 1-(3-(2-phenyl)propylamino)propyl)-1H-indole-3-carboxylate C780

Amount made: 8.2 mg. LCMS m/z 351 [M+H]⁺, purity (UV/MS) 88/80.

### methyl 1-(3-(3,4-dihydroisoquinolin-2(1H)-yl)propyl)-1H-indole-3-carboxylate C781

Amount made: 12.8 mg. LCMS m/z 349 [M+H]⁺, purity (UV/MS) 93/69.

### methyl 1-(3-(3-acetamidopyrrolidin-1-yl)propyl)-1H-indole-3-carboxylate C782

Amount made: 12.2 mg. LCMS m/z 344 [M+H]⁺, purity (UV/MS) 99/93.

### methyl 1-(3-(4-((tetrahydrofuran-2-yl)methyl)piperazin-1-yl)propyl)-1H-indole-3-carboxylate C783

Amount made: 14.2 mg. LCMS m/z 386 [M+H]⁺, purity (UV/MS) 97/78.

### methyl 1-(3-(4-(1H-indol-4-yl)piperazin-1-yl)propyl)-1H-indole-3-carboxylate C784

Amount made: 20.9 mg. LCMS m/z 417 [M+H]⁺, purity (UV/MS) 77/43.

### methyl 1-(3-(4-(1-phenylethyl)piperazin-1-yl)propyl)-1H-indole-3-carboxylate C785

Amount made: 14.5 mg. LCMS m/z 406 [M+H]⁺, purity (UV/MS) 97/93.

### methyl 1-(3-(4-(2-(diisopropylamino)ethyl)piperazin-1-yl)propyl)-1H-indole-3-carboxylate C786

Amount made: 17.7 mg. LCMS m/z 429 [M+H]⁺, purity (UV/MS) 95/65.

### methyl 1-(3-(4-(2-(dimethylamino)-2-oxoethyl)piperazin-1-yl)propyl)-1H-indole-3-carboxylate C787

Amount made: 18.7 mg. LCMS m/z 387 [M+H]⁺, purity (UV/MS) 97/73.

### methyl 1-(3-(4-(2-(dimethylamino)ethyl)piperazin-1-yl)propyl)-1H-indole-3-carboxylate C788

Amount made: 10.1 mg. LCMS m/z 373 [M+H]⁺, purity (UV/MS) 81/41.

### methyl 1-(3-(4-(2-(methylthio)phenyl)piperazin-1-yl)propyl)-1H-indole-3-carboxylate C789

Amount made: 20.1 mg. LCMS m/z 424 [M+H]⁺, purity (UV/MS) 85/57.

### methyl 1-(3-(4-(2,4-difluorobenzoyl)piperidin-1-yl)propyl)-1H-indole-3-carboxylate C790

Amount made: 21.1 mg. LCMS m/z 441 [M+H]⁺, purity (UV/MS) 80/64.

### methyl 1-(3-(4-(2-chlorophenyl)piperazin-1-yl)propyl)-1H-indole-3-carboxylate hydrochloride C791

Amount made: 16.7 mg. LCMS m/z 412 [M+H]⁺, purity (UV/MS) 88/54.

### methyl 1-(3-(4-(2-ethoxyethyl)piperazin-1-yl)propyl)-1H-indole-3-carboxylate C792

Amount made: 15.7 mg. LCMS m/z 374 [M+H]⁺, purity (UV/MS) 95/70.

### methyl 1-(3-(4-(2-hydroxyphenyl)piperazin-1-yl)propyl)-1H-indole-3-carboxylate C793

Amount made: 2.2 mg. LCMS m/z 394 [M+H]⁺, purity (UV/MS) 94/74.

### methyl 1-(3-(4-(2-methoxyethyl)piperazin-1-yl)propyl)-1H-indole-3-carboxylate C794

Amount made: 9.3 mg. LCMS m/z 360 [M+H]⁺, purity (UV/MS) 96/79.

### methyl 1-(3-(4-(2-methoxyphenyl)piperidin-1-yl)propyl)-1H-indole-3-carboxylate C795

Amount made: 19.4 mg. LCMS m/z 407 [M+H]⁺, purity (UV/MS) 98/73.

### methyl 1-(3-(4-(2-morpholino-2-oxoethyl)piperazin-1-yl)propyl)-1H-indole-3-carboxylate C796

Amount made: 15.7 mg. LCMS m/z 429 [M+H]⁻, purity (UV/MS) 94/79.

### methyl 1-(3-(4-(3-chlorophenyl)piperazin-1-yl)propyl)-1H-indole-3-carboxylate C797

Amount made: 15.1 mg. LCMS m/z 412 [M+H]⁺, purity (UV/MS) 84/51.

### methyl 1-(3-(4-(3-cyanopyridin-2-yl)-1,4-diazepan-1-yl)propyl)-1H-indole-3-carboxylate C798

Amount made: 5.8 mg. LCMS m/z 418 [M+H]⁺, purity (UV/MS) 93/83.

### methyl 1-(3-(4-(4-chlorophenyl)piperazin-1-yl)propyl)-1H-indole-3-carboxylate C799

Amount made: 15.5 mg. LCMS m/z 412 [M+H]⁺, purity (UV/MS) 80/48.

### methyl 1-(3-(4-(4-cyanophenyl)piperazin-1-yl)propyl)-1H-indole-3-carboxylate hydrochloride C800

Amount made: 5.5 mg. LCMS m/z 403 [M+H]⁺, purity (UV/MS) 100/100.

### methyl 1-(4-(4-fluoro-2-nitrophenyl)piperazin-1-yl)propyl)-1H-indole-3-carboxylate C801

Amount made: 22.0 mg. LCMS m/z 441 [M+H]⁺, purity (UV/MS) 77/47.

### methyl 1-(3-(4-(4-fluorophenoxy)piperidin-1-yl)propyl)-1H-indole-3-carboxy late hydrochloride C802

Amount made: 17.5 mg. LCMS m/z 411 [M+H]⁺, purity (UV/MS) 95/86.

### methyl 1-(3-(4-(6-fluorobenzo[d]isoxazol-3-yl)piperidin-1-yl)propyl)-1H-indole-3-carboxylate C803

Amount made: 15.1 mg. LCMS m/z 436 [M+H]⁺, purity (UV/MS) 87/78.

### methyl 1-(3-(4-(pyrrolidin-1-yl)piperidin-1-yl)propyl)-1H-indole-3-carboxylate C804

Amount made: 26.9 mg. LCMS m/z 370 [M+H]⁺, purity (UV/MS) 99/84.

### methyl 1-(3-(4-phenylpiperazin-1-yl)propyl)-1H-indole-3-carboxylate C805

Amount made: 17.5 mg. LCMS m/z 378 [M+H]⁺, purity (UV/MS) 87/68.

### methyl 1-(3-(4-phenylpiperidin-1-yl)propyl)-1H-indole-3-carboxylate C806

Amount made: 13.2 mg. LCMS m/z 377 [M+H]⁺, purity (UV/MS) 97/79.

### methyl 1-(3-(methyl(2-(pyridin-2-yl)ethyl)amino)propyl)-1H-indole-3-carboxylate C807

Amount made: 12.1 mg. LCMS m/z 352 [M+H]⁺, purity (UV/MS) 93/72.

### 1-(3-((2-(1H-indol-3-yl)ethyl)(methyl)amino)propyl)-N-methoxy-N-methyl-1H-indole-3-carboxamide C808

Amount made: 0.8 mg. LCMS m/z 419 [M+H]⁺, purity (UV/MS) 80/60.

### 1-(3-(3,4-dihydroisoquinolin-2(1H)-ylpropyl)-N,5-dimethoxy-N-methyl-1H-indole-3-carboxamide C809

Amount made: 23.2 mg. LCMS m/z 408 [M+H]⁺, purity (UV/MS) 100/66.

### 1-(3-(3-acetamidopyrrolidin-1-yl)propyl)-N-methoxy-N-methyl-1H-indole-3-carboxamide C810

Amount made-7.3 mg. LCMS m/z 373 [M+H]⁺, purity (UV/MS) 88/70.

### 1-(3-(4-(2-(diisopropylamino)ethyl)piperazin-1-yl)propyl)-N-methoxy-N-methyl-1H-indole-3-carboxamide C811

Amount made: 9.8 mg. LCMS m/z 458 [M+H]⁺, purity (UV/MS) 99/80.

### 1-(3-(4-(2-(dimethylamino)-2-oxoethyl)piperazin-1-yl)propyl)-N-methoxy-N-methyl-1H-indole-3-carboxamide C812

Amount made: 11.8 mg. LCMS m/z 416 [M+H]⁺, purity (UV/MS) 94/85.

### 1-(3-(4-(2-(dimethylamino)ethyl)piperazin-1-yl)propyl)-N-methoxy-N-methyl-1H-indole-3-carboxamide C813

Amount made: 6.9 mg. LCMS m/z 402 [M+H]⁺, purity (UV/MS) 76/55.

### 1-(3-(4-(2-chlorophenyl)piperazin-1-yl)propyl)-N,5-dimethoxy-N-methyl-1H-indole-3-carboxamide hydrochloride C814

Amount made: 7.2 mg. LCMS m/z 471 [M+H]⁺, purity (UV/MS) 90/70.

### 1-(3-(4-(2-chlorophenyl)piperazin-1-yl)propyl)N-methoxy-N-methyl-1H-indole-3-carboxamide hydrochloride C815

Amount made: 2.3 mg. LCMS m/z 441 [M+H]⁺, purity (UV/MS) 90/79.

### 1-(3-(4-(2-ethoxyethyl)piperazin-1-yl)propyl)-N-methoxy-N-methyl-1H-indole-3-carboxamide C816

Amount made: 4.8 mg. LCMS m/z 403 [M+H]⁺, purity (UV/MS) 95/77.

### 1-(3-(4-(2-hydroxyphenyl)piperazin-1-yl)propyl)-N,5-dimethoxy-N-methyl-1H-indole-3-carboxamide C817

Amount made: 7.1 mg. LCMS m/z 453 [M+H]⁺, purity (UV/MS) 89/70.

### 1-(3-(4-(2-hydroxyphenyl)piperazin-1-yl)propyl)-N-methoxy-N-methyl-1H-indole-3-carboxamide C818

Amount made: 5.9 mg. LCMS m/z 423 [M+H]⁺, purity (UV/MS) 80/70.

### 1-(3-(4-(3-chlorophenyl)piperazin-1-yl)propyl)-N-methoxy-N-methyl-1H-indole-3-carboxamide C819

Amount made: 2.4 mg. LCMS m/z 441 [M+H]⁺, purity (UV/MS) 86/76.

### 1-(3-(4-(3-cyanopyridin-2-yl)-1,4-diazepan-1-yl)propyl)-N-methoxy-N-methyl-1H-indole-3-carboxamide C820

Amount made: 5.4 mg. LCMS m/z 447 [M+H]⁺, purity (UV/MS) 95/65.

### 1-(3-(4-(4-acetylphenyl)piperazin-1-yl)propyl)-N-methoxy-N-methyl-1H-indole-3-carboxamide C821

Amount made: 14.8 mg. LCMS m/z 449 [M+H]⁺, purity (UV/MS) 96/70.

### 1-(3-(4-(4-chlorophenyl)piperazin-1-yl)propyl)N-methoxy-N-methyl-1H-indole-3-carboxamide C822

Amount made: 11.4 mg. LCMS m/z 441 [M+H]⁺, purity (UV/MS) 90/70.

### 1-(3-(4-(4-chlorophenylsulfonyl)piperidin-1-yl)propyl)-N-methoxy-N-methyl-1H-indole-3-carboxamide hydrochloride C823

Amount made: 6.8 mg. LCMS m/z 504 [M+H]⁺, purity (UV/MS) 96/73.

### 1-(3-(4-(4-chlorophenylthio)piperidin-1-yl)propyl)-N-methoxy-N-methyl-11H-indole-3-carboxamide hydrochloride C824

Amount made: 8.4 mg. LCMS m/z 472 [M+H]⁺, purity (UV/MS) 97/72.

### 1-(3-(4-(4-cyanophenyl)piperazin-1-yl)propyl)-N-methoxy-N-methyl I H-indole-3-carboxamide hydrochloride C825

Amount made: 1.7 mg. LCMS m/z 432 [M+H]⁺, purity (UV/MS) 90/60.

### 1-(3-(4-(4-fluoro-2-nitrophenyl)piperazin-1-yl)propyl)-N-methoxy-N-methyl-1H-indole-3-carboxamide C826

Amount made: 1.6 mg. LCMS m/z 470 [M+H]⁺, purity (UV/MS) 84/55.

### 1-(3-(4-(4-fluorophenoxy)piperidin-1-yl)propyl)-N-methoxy-N-methyl-1H-indole-3-carboxamide hydrochloride C827

Amount made: 10.9 mg. LCMS m/z 440 [M+H]⁺, purity (UV/MS) 90/70.

### 1-(3-(4-(4-fluorophenyl)piperazin-1-yl)propyl)-N,5-dimethoxy-N-methyl-1H-indole-3-carboxamide C828

Amount made: 12.4 mg. LCMS m/z 455 [M+H]⁺, purity (UV/MS) 93/70.

### 1-(3-(4-(4-fluorophenyl)piperazin-1-yl)propyl)-N-methoxy-N-methyl-1H-indole-3-carboxamide C829

Amount made: 6.3 mg. LCMS m/z 425 [M+H]⁺, purity (UV/MS) 88/60.

### 1-(3-(4-benzylpiperidin-1-yl)propyl)-N,5-dimethoxy-N-methyl-1H-indole-3-carboxamide C830

Amount made: 18.6 mg. LCMS m/z 450 [M+H]⁺, purity (UV/MS) 98/70.

### 1-(3-(4-butylpiperidin-1-yl)propyl)-N,5-dimethoxy-N-methyl-1H-indole-3-carboxamide C831

Amount made: 16.3 mg. LCMS m/z 416 [M+H]⁺, purity (UV/MS) 100/84.

### N,5-dimethoxy-N-methyl-1-(3-(4-(2-morpholino-2-oxoethyl)piperazin-1-yl)propyl)-1H-indole-3-carboxamide C832

Amount made: 20.4 mg. LCMS m/z 488 [M+H]⁺, purity (UV/MS) 95/60.

### N,5-dimethoxy-N-methyl-1-(3-(4-(pyrrolidin-1-yl)piperidin-1-yl)propyl)-1H-indole-3-carboxamide C833

Amount made: 17.6 mg. LCMS m/z 429 [M+H]⁺, purity (UV/MS) 95/92.

### N,5-dimethoxy-N-methyl-1-(3-(4-phenylpiperazin-1-yl)propyl)-1H-indole-3-carboxamide

Amount made: 14.0 mg. LCMS m/z 437 [M+H]⁺, purity (UV/MS) 88/50.

### N,5-dimethoxy-N-methyl-1-(3-(octahydroisoquinolin-2(1H)-yl)propyl)-1H-indole-3-carboxamide C835

Amount made: 15.0 mg. LCMS m/z 414 [M+H]⁺, purity (UV/MS) 95/67.

### N-methoxy-1-(3-(4-(2-methoxyethyl)piperazin-1-yl)propyl)-N-methyl-1H-indole-3-carboxamide C836

Amount made: 9.4 mg. LCMS m/z 389 [M+H]⁺, purity (UV/MS) 96/82.

### N-methoxy-N-methyl-1-(3-(4-((tetrahydrofuran-2-yl)methyl)piperazin-1-yl)propyl)-1H-indole-3-carboxamide C837

Amount made: 8.1 mg. LCMS m/z 415 [M+H]⁺, purity (UV/MS) 95/81.

### N-methoxy-N-methyl-1-(3-(4-(2-(methylthio)phenyl)piperazin-1-yl)propyl)-1H-indole-3-carboxamide C838

Amount made: 6.0 mg. LCMS m/z 453 [M+H]⁺, purity (UV/MS) 86/70.

### N-methoxy-N-methyl-1-(3-(4-(2-morpholino-2-oxoethyl)piperazin-1-yl)propyl) I H-indole-3-carboxamide C839

Amount made: 13.7 mg. LCMS m/z 458 [M+H]⁺, purity (UV/MS) 95/73.

### N-methoxy-N-methyl-1-(3-(4-(pyrrolidin-1-yl)piperidin-1-yl)propyl)-1H-indole-3-carboxamide C840

Amount made: 8.0 mg. LCMS m/z 399 [M+H]⁺, purity (UV/MS) 92/70.

### N-methoxy-N-methyl-1-(3-(4-phenylpiperidin-1-yl)propyl)-1H-indole-3-carboxamide C841

Amount made: 4.1 mg. LCMS m/z 406 [M+H]⁺, purity (UV/MS) 96/80.

### N-methoxy-N-methyl-1-(3-(methyl(2-(pyridin-2-yl)ethyl)amino)propyl)-1H-indole-3-carboxamide C842

Amount made: 7.1 mg. LCMS m/z 381 [M+H]⁺, purity (UV/MS) 98/84.

### 1-(1-(3-(2,3-dihydro-1H-inden-2-ylamino)propyl)-1H-pyrrolo[2,3-b]pyridin-3-yl)-2,2,2-trifluoroethanone C843

Amount made: 1.9 mg. LCMS m/z 388 [M+H]⁺, purity (UV/MS) 79/50.

### 1-(1-(3-(4-(3-chloro-5-(trifluoromethyl)pyridin-2-yl)piperazin-1-yl)propyl)-1H-pyrrolo[2,3-b]pyridin-3-yl)-2,2,2-trifluoroethanone C844

Amount made: 2.6 mg. LCMS m/z 520 [M+H]⁺, purity (UV/MS) 98/80.

### 1-(1-(3-(4-(2-(4-chlorophenoxy)ethyl)piperazin-1-yl)propyl)-1H-pyrrolo[2,3-b]pyridin-3-yl)-2,2,2-trifluoroethanone C845

Amount made: 1.8 mg. LCMS m/z 495 [M+H]⁺, purity (UV/MS) 99/80.

### 2,2,2-trifluoro-1-(1-(3-(2-phenoxyethylamino)propyl)-1H-pyrrolo[2,3-b]pyridin-3-yl)ethanone C846

Amount made: 0.8 mg. LCMS m/z 392 [M+H]⁺, purity (UV/MS) 100/80.

### 2,2,2-trifluoro-1-(1-(3-(4-(2-phenoxyethyl)piperazin-1-yl)propyl)-1H-pyrrolo[2,3-b]pyridin-3-yl)ethanone C847

Amount made: 3.0 mg. LCMS m/z 461 [M+H]⁺, purity (UV/MS) 100/90.

### 2,2,2-trifluoro-1-(1-(3-(4-phenethyl-1,4-diazepan-1-yl)propyl)-1H-pyrrolo[2,3-b]pyridin-3-yl)ethanone C848

Amount made: 2.6 mg. LCMS m/z 459 [M+H]⁺, purity (UV/MS) 99/80.

### 2,2,2-trifluoro-1-(1-(3-(4-(4-fluorobenzyl)-1,4-diazepan-1-yl)propyl)-1H-pyrrolo[2,3-b]pyridin-3-yl)ethanone C849

Amount made: 2.2 mg. LCMS m/z 463 [M+H]⁺, purity (UV/MS) 100/80.

### 1-(1-(3-(4-(2,4-dichlorobenzyl)piperazin-1-yl)propyl)-1H-pyrrolo[2,3-b]pyridin-3-yl)-2,2,2-trifluoroethanone C850

Amount made: 2.9 mg. LCMS m/z 499 [M+H]⁺, purity (UV/MS) 99/80.

### 2,2,2-trifluoro-1-(1-(3-(4-(3-(pyridin-4-yl)-1,2,4-oxadiazol-5-yl)piperidin-1-yl)propyl)-1H-pyrrolo[2,3-b]pyridin-3-yl)ethanone C851

Amount made: 3.1 mg. LCMS m/z 485 [M+H]⁺, purity (UV/MS) 99/90.

### 2,2,2-trifluoro-1-(1-(3-(4-(3-(pyridin-3-yl)-1,2,4-oxadiazol-5-yl)piperidin-1-yl)propyl)-1H-pyrrolo[2,3-b]pyridin-3-yl)ethanone C852

Amount made: 1.9 mg. LCMS m/z 485 [M+H]⁺, purity (UV/MS) 99/90.

### 1-(1-(3-(4-(3-chloro-5-(trifluoromethyl)pyridin-2-yl)piperazin-1-yl)propyl)-1H-pyrrolo[2,3-b]pyridin-3-yl)ethanone C853

Amount made: 0.6 mg. LCMS m/z 466 [M+H]⁺, purity (UV/MS) 100/90.

### 1-(1-(3-(4-(2-(4-chlorophenoxy ethyl)piperazin-1-yl)propyl)-1H-pyrrolo[2,3-h]pyridin-3-yl)ethanone C854

Amount made: 1.6 mg. LCMS m/z 441 [M+H]⁺, purity (UV/MS) 100/80.

### 1-(1-(3-(4-(2-phenoxyethyl)piperazin-1-yl)propyl)-1H-pyrrolo[2,3-b]pyridin-3-yl)ethanone C855

Amount made; 0.2 mg. LCMS m/z 407 [M+H]⁺, purity (UV/MS) 100/80.

### 1-(1-(3-(4-phenethyl-1,4-diazepan-1-yl)propyl)-1H-pyrrolo[2,3-b]pyridin-3-yl)ethanone C856

Amount made: 0.7 mg. LCMS m/z 405 [M+H]⁺, purity (UV/MS) 100/80.

### 1-(1-(3-(4-(4-fluorobenzyl)-1,4-diazepan-1-yl)propyl)-1H-pyrrolo[2,3-b]pyridin-3-yl)ethanone C857

Amount made: 1.6 mg. LCMS m/z 409 [M+H]⁺, purity (UV/MS) 100/80.

### 1-(1-(3-(4-(2,4-dichlorobenzyl)piperazin-1-yl)propyl)-1H-pyrrolo[2,3-b]pyridin-3-yl)ethanone C858

Amount made: 0.7 mg. LCMS m/z 445 [M+H]⁺, purity (UV/MS) 100/80.

### 1-(1-(3-(4-(3-(pyridin-4-yl)-1,24-oxadiazol-5-yl)piperidin-1-yl)propyl)-1H-pyrrolo[2,3-b]pyridin-3-yl)ethanone C859

Amount made: 0.8 mg. LCMS m/z 431 [M+H]⁺, purity (UV/MS) 97/70.

### 1-(1-(3-(4-(3-(pyridin-3-yl)-1,2,4-oxadiazol-5-yl)piperidin-1-yl)propyl)-1H-pyrrolo[2,3-b]pyridin-3-yl)ethanone C860

Amount made: 0.2 mg. LCMS m/z 431 [M+H]⁺, purity (UV/MS) 99/70.

### 1′-(3-(3-(2,2,2-trifluoroacetyl)-1H-pyrrolo[2,3-b]pyridin-1-yl)propyl)spiro[chroman-2,4′-piperidin]-4-one C861

Amount made: 1.0 mg. LCMS m/z 472 [M+H]⁺, purity (UV/MS) 99/90.

### 1-(1-(3-(4-(2-chlorobenzyl)-1,4-diazepan-1-yl)propyl)-1H-pyrrolo[2,3-b]pyridin-3-yl)-2,2,2-trifluoroethanone C862

Amount made: 2.5 mg. LCMS m/z 479 [M+H]⁺, purity (UV/MS) 99/90.

### 1-(1-(3-(3-(4-chlorophenoxy)piperidin-1-yl)propyl)-1H-pyrrolo[2,3-b]pyridin-3-yl)-2,2,2-trifluoroethanone C863

Amount made: 1.0 mg. LCMS m/z 466 [M+H]⁺, purity (UV/MS) 99/90.

### 1-(1-(3-(3-(2-chlorobenzyl)piperidin-1-yl)propyl)-1H-pyrrolo[2,3-b]pyridin-3-yl)-2,2,2-trifluoroethanone C864

Amount made: 0.8 mg. LCMS m/z 464 [M+H]⁺, purity (UV/MS) 100/90.

### 2,2,2-trifluoro-1-(1-(3-(3-phenethyl-8-azabicyclo[3.2.1]octan-8-yl)propyl)-1H-pyrrolo[2,3-b]pyridin-3-yl)ethanone C865

Amount made: 1.7 mg. LCMS m/z 470 [M+H]⁺, purity (UV/MS) 97/70.

### 1-(1-(3-(3-(4-chlorophenethyl)-8-azabicyclo[3.2.1]octan-8-yl)propyl)-1H-pyrrolo[2,3-b]pyridin-3-yl)-2,2,2-trifluoroethanone C866

Amount made: 3.2 mg. LCMS m/z 504 [M+H]⁺, purity (UV/MS) 93/60.

### 1-(1-(3-(3-(2-(4-chlorophenyl)-2-oxoethyl)-8-azabicyclo[3.2.1]octan-8-yl)propyl)-1H-pyrrolo[2,3-b]pyridin-3-yl)-2,2,2-trifluoroethanone C867

Amount made: 2.6 mg. LCMS m/z 518 [M+H]⁺, purity (UV/MS) 94/80.

### 1-(1-(3-(4-(3-chlorophenoxy)-piperidin-1-yl)propyl)-1H-pyrrolo[2,3-b]pyridin-3-yl)-2,2,2-trifluoroethanone C868

Amount made: 0.8 mg. LCMS m/z 466 [M+H]⁺, purity (UV/MS) 100/90.

### 2,2,2-trifluoro-1-(1-(3-(3-(4-fluorophenoxy)-8-azabicyclo[3.2.1]octan-8-yl)propyl)-1H-pyrrolo[2,3-b]pyridin-3-yl)ethanone C869

Amount made: 0.9 mg. LCMS m/z 476 [M+H]⁺, purity (UV/MS) 100/70.

### 1-(1-(3-(3-(2-chlorophenoxy)-8-azabicyclo[3.2.1]octan-8-yl)propyl)-1H-pyrrolo[2,3-b]pyridin-3-yl)-2,2,2-trifluoroethanone C870

Amount made: 1.7 mg. LCMS m/z 492 [M+H]⁺, purity (UV/MS) 100/90.

### 1-(1-(3-(4-(2-(4-chlorophenoxy)ethyl)piperidin-1-yl)propyl)-1H-pyrrolo[2,3-b]pyridin-3-yl)-2,2,2-trifluoroethanone C871

Amount made: 0.8 mg. LCMS m/z 494 [M+H]⁺, purity (UV/MS) 100/90.

### 1-(1-(3-(4-(2-chlorobenzyl)-1,4-diazepan-1-yl)propyl)-1H-pyrrolo[2,3-b]pyridin-3-yl)ethanone C872

Amount made: 1.7 mg. LCMS m/z 425 [M+H]⁺, purity (UV/MS) 90/50.

### 1-(1-(3-(3-(4-chlorophenoxy)piperidin-1-yl)propyl)-1H-pyrrolo[2,3-b]pyridin-3-yl)ethanone C873

Amount made: 0.3 mg. LCMS m/z 412 [M+H]⁺, purity (UV/MS) 100/90.

### 1-(1-(3-(3-(4-chlorophenethyl)-8-azabicyclo[3.2.1]octan-8-yl)propyl)-1H-pyrrolo[2,3-b]pyridin-3-yl)ethanone C874

Amount made: 1.7 mg. LCMS m/z 450 [M+H]⁺, purity (UV/MS) 100/80.

### 2-(8-(3-(3-acetyl-1H-pyrrolo[2,3-b]pyridin-1-yl)propyl)-8-azabicyclo[3.2.1]octan-3-yl)-1-(4-chlorophenyl)ethanone C875

Amount made: 1.6 mg. LCMS m/z 464 [M+H]⁺, purity (UV/MS) 100/90.

### 1-(1-(3-(4-(3-chlorophenoxy)piperidin-1-yl)propyl)-1H-pyrrolo[2,3-b]pyridin-3-yl)ethanone C876

Amount made: 1.5 mg. LCMS m/z 412 [M+H]⁺, purity (UV/MS) 99/90.

### 1-(1-(3-(4-fluorophenoxy)-8-azabicyclo[3.2.1]octan-8-yl)propyl)-1H-pyrrolo[2,3-b]pyridin-3-yl)ethanone C877

Amount made: 0.6 mg. LCMS m/z 422 [M+H]⁺, purity (UV/MS) 100/100.

### 1-(1-(3-(3-(2-chlorophenoxy)-1-azabicyclo[3.2.1]octan-8-yl)propyl)-1H-pyrrolo[2,3-b]pyridin-3-yl)ethanone C878

Amount made: 1.1 mg. LCMS m/z 438 [M+H]⁺, purity (UV/MS) 100/90.

### 1-(1-(3-(4-(2-C4-chlorophenoxy)ethyl)piperidin-1-yl)propyl)-1H-pyrrolo[2,3-b]pyridin-3-yl)ethanone C879

Amount made: 0.8 mg. LCMS m/z 440 [M+H]⁺, purity (UV/MS) 100/90.

### 1-(3-(4-(3-chloro-5-(trifluoromethyl)pyridin-2-yl)piperazin-1-yl)propyl)-7-methoxy-1H-indole-3-carbonitrile C880

Amount made: 4.5 mg. LCMS m/z 478 [M+H]⁺, purity (UV/MS) 100/100.

### 1-(3-(4-(2-(4-chlorophenoxy)ethyl)piperazin-1-yl)propyl)-7-methoxy-1H-indole-3-carbonitrile C881

Amount made: 3.1 mg. LCMS m/z 453 [M+H]⁺, purity (UV/MS) 100/90.

### 7-methoxy-1-(3-(4-(2-phenoxyethyl)piperazin-1-yl)propyl)-1H-indole-3-carbonitrile C882

Amount made: 3.2 mg. LCMS m/z 419 [M+H]⁺, purity (UV/MS) 100/90.

### 1-(3-(4-C4-fluorobenzyl)-1,4-diazepan-1-yl)propyl)-7-methoxy-1H-indole-3-carbonitrile C883

Amount made: 1.4 mg. LCMS m/z 421 [M+H]⁺, purity (UV/MS) 99/80.

### 1-(3-(4-(2,4-dichlorobenzyl)piperazin-1-yl)propyl)-7-methoxy-1H-indole-3-carbonitrile C884

Amount made: 4.9 mg. LCMS m/z 457 [M+H]⁺, purity (UV/MS) 100/90.

### 1-(3-(4-(2-(4-chloronaphthalen-1-yloxy)ethyl)piperazin-1-yl)propyl)-7-methoxy-1H-indole-3-carbonitrile C885

Amount made: 1.0 mg. LCMS m/z 503 [M+H]⁺, purity (UV/MS) 100/100.

### 7-methoxy-1-(3-(4-(3-(pyridin-4-yl)-1,2,4-oxadiazol-5-yl)piperidin-1-yl)propyl)-1H-indole-3-carbonitrile C886

Amount made: 4.0 mg. LCMS m/z 443 [M+H]⁺, purity (UV/MS) 100/90.

### 7-methoxy-1-(3-(4-(3-(pyridin-3-yl)-1,2,4-oxadiazol-5-yl)piperidin-1-yl)propyl)-1H-indole-3-carbonitrile C887

Amount made: 3.9 mg. LCMS m/z 443 [M+H]⁺, purity (UV/MS) 99/80.

### 7-methoxy-1-(3-(4-(2-oxoindolin-1-yl)piperidin-1-yl)propyl)-1H-indole-3-carbonitrile C888

Amount made: 2.6 mg. LCMS m/z 429 [M+H]⁺, purity (UV/MS) 93/80.

### 7-methoxy-1-(3-(4-oxospiro[chroman-2,4′-piperidine]-1′-yl)propyl)-1H-indole-3-carbonitrile C889

Amount made: 2.3 mg. LCMS m/z 430 [M+H]⁺, purity (UV/MS) 100/100.

### 1-(3-(4-(2-chlorobenzyl)-1,4-diazepan-1-yl)propyl)-7-methoxy-1H-indole-3-carbonitrile C890

Amount made: 3.1 mg. LCMS m/z 437 [M+H]⁺, purity (UV/MS) 97/80.

### 1-(3-(3-(4-chlorophenoxy)piperidin-1-yl)propyl)-7-methoxy-1H-indole-3-carbonitrile C891

Amount made: 3.4 mg. LCMS m/z 424 [M+H]⁺, purity (UV/MS) 100/90.

### 1-(3-(3-(2-chlorobenzyl)piperidin-1-yl)propyl)-7-methoxy-1H-indole-3-carbonitrile C892

Amount made: 3.8 mg. LCMS m/z 422 [M+H]⁺, purity (UV/MS) 100/100.

### 7-methoxy-1-(3-(3-phenethyl-8-azabicyclo[3.2.1]octan-8-yl)propyl)-1H-indole-3-carbonitrile C893

Amount made: 2.6 mg. LCMS m/z 428 [M+H]⁺, purity (UV/MS) 94/80.

### 1-(3-(3-(4-chlorophenethyl)-8-azabicyclo[3.2.1]octan-8-yl)propyl)-7-methoxy-1H-indole-3-carbonitrile C894

Amount made: 4.3 mg. LCMS m/z 462 [M+H]⁺, purity (UV/MS) 98/80.

### 1-(3-(3-(2-(4-chlorophenyl)-2-oxoethyl)-8-azabicyclo[3.2.1]octan-8-yl)propyl)-7-methoxy-1H-indole-3-carbonitrile C895

Amount made: 5.2 mg. LCMS m/z 476 [M+H]⁺, purity (UV/MS) 98/80.

### 1-(3-(4-(3-chlorophenoxy)piperidin-1-yl)propyl)-7-methoxy-1H-indole-3-carbonitrile C896

Amount made: 4.0 mg. LCMS m/z 424 [M+H]⁺, purity (UV/MS) 100/100.

### 1-(3-(3-(4-fluorophenoxy)-8-azabicyclo[3.2.1]octan-8-yl)propyl)-7-methoxy-1H-indole-3-carbonitrile C897

Amount made: 3.2 mg. LCMS m/z 434 [M+H]⁺, purity (UV/MS) 95/80.

### 1-(3-(3-(2-chlorophenoxy)-8-azabicyclo[3.2.1]octan-8-yl)propyl)-7-methoxy-1H-indole-3-carbonitrile C898

Amount made: 3.5 mg. LCMS m/z 450 [M+H]⁺, purity (UV/MS) 100/90.

### 1-(3-(4-(2-(4-chlorophenoxy)ethyl)piperidin-1-yl)propyl)-7-methoxy-1H-indole-3-carbonitrile C899

Amount made: 1.2 mg. LCMS m/z 452 [M+H]⁺, purity (UV/MS) 100/90.

### 1-(1-(3-(4-butylpiperidin-1-yl)propyl)-1H-indol-3-yl)ethanone C900

Amount made: 16.2 mg. LCMS m/z 341 [M+H]⁺, purity (UV/MS) 99/100.

### 1-(1-(3-(6-methoxy-3,4-dihydro-1H-pyrido[3,4-b]indol-2(9H)-yl)propyl)-1H-indol-3-yl)ethanone C901

Amount made: 3.0 mg. LCMS m/z 402 [M+H]⁺, purity (UV/MS) 100/82.

### 1-(7-methoxy-1-(3-(4-(3-(trifluoromethyl)pyridin-2-yl)piperazin-1-yl)propyl)-1H-indol-3-yl)ethanone C902

Amount made: 9.6 mg. LCMS m/z 463 [M+H]⁺, purity (UV/MS) 91/60.

### N-(1-(3-(3-acetyl-7-methoxy-1H-indol-1-yl)propyl)pyrrolidin-3-yl)-N-methylacetamide C903

Amount made: 6.4 mg. LCMS m/z 374 [M+H]⁺, purity (UV/MS) 90/60.

### 8-(3-(3-acetyl-7-methoxy-1H-indol-1-yl)propyl)-1-phenyl-1,38-triazaspiro[4,5]decan-4-one C904

Amount made: 1.1 mg. LCMS m/z 463 [M+H]⁺, purity (UV/MS) 82/60.

### N-(1-(3-(3-acetyl-7-methoxy-1H-indol-1-yl)propyl)piperidin-4-yl)-N-cyclopropylbenzenesulfonamide C905

Amount made: 9.1 mg. LCMS m/z 512 [M+H]⁺, purity (UV/MS) 94/50.

### General procedure for substitution on the tail (GP2): 1-[1-[3-[3-butyl-8-aza-bicyclo[3.2.1]octan-8-yl]propyl]-1H-indol-3-yl]ethanone

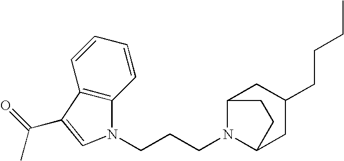

1-[1-(3-chloropropyl)-1H-indol-3-yl]-ethanone (12 mg, 0.05 mmol), cesium carbonate (32 mg, 0.1 mmol), potassium iodide (8 mg, 0.05 mmol) and 5-butyl-2-azabicyclo[2.2.1]heptane (6 mg, 0.04 mmol) were weighed into a vial, dry MeCN (1 mL) was added and the reaction mixture was shaken at 80° C. on a shaker over night. The product was purified by SCX to yield the title compound.

LC/MS purity: UV/MS 88/67

### 1-[1-[2-[3-(cyclopropylmethoxy)-8-azabicyclo[3.2.1]oct-8-yl]propyl]-1H-indol-3-yl]-ethanone

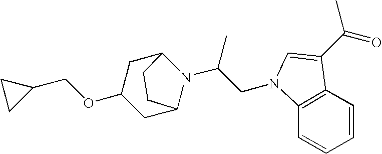

Prepared according to GP2 from 1-[1-(3-chloropropyl)-1H-indol-3-yl]-ethanone (12 mg, 0.05 mmol), cesium carbonate (32 mg, 0.1 mmol), potassium iodide (8 mg, 0.05 mmol) and 3-(cyclopropylmethoxy)-8-azabicyclo[3.2.1]octane (7 mg, 0.04 mmol).

LC/MS purity: UV/MS 99/88

### General procedure for amide formation (GP3): N-phenyl-1H-indole-3-carboxamide

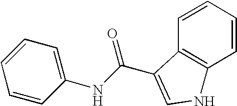

Indole-3-carboxylic acid (644 mg, 4 mmol), 1-hydroxybenzothiazole (810 mg, 6 mmol), 1-ethyl-3-(3-dimethylaminopropyl)carbodimide hydrochloride (1.15 g, 6 mmol), DMAP (5 mg, 0.04 mmol), triethyl amine (1.84 g, 18 mmol) and 4-chlorobenzyl amine (566 mg, 4 mmol) were weighed into a MW vial, dry MeCN (10 mL) was added, and the reaction heated in the MW at 140° C. for 15 min. The reaction mixture was diluted with EtOAc and washed with water and brine, dried over sodium sulphate, filtered and concentrated in vacuo. The product was recrystallized from methanol. Yield: 733 mg (65%).

Receptor Selection and Amplification Technology (R-SAT) Assay

The functional receptor assay, Receptor Selection and Amplification Technology (R-SAT), was used to investigate the pharmacological properties of novel compounds. R-SAT is disclosed in U.S. Pat. Nos. 5,707,798, 5,912,132, and 5,955,281, all of which are hereby incorporated herein by reference in their entirety, including any drawings. These experiments have provided a molecular profile, or fingerprint, for each of these agents.

Briefly, NIH3T3 cells were grown in 96 well tissue culture plates to 70-80% confluence. Cells were transfected for 16-20 h with plasmid DNAs using Polyfect (Qiagen Inc.) using the manufacturer's protocols. R-SATs were generally performed with 20 ng/well of receptor, 10 ng/well of RGS1 (Burstein et al., JPET, 315:1278-1287) and 20 ng/well of β-galactosidase plasmid DNA. All receptor constructs used were in the pS1-derived mammalian expression vector (Promega Inc). The Ghrelin receptor genes were amplified by PCR from genomic DNA using oligodeoxynucleotide primers based on the published sequences. For large-scale transfections, cells were transfected for 16-20 h, then trypsinized and frozen in DMSO. Frozen cells were later thawed, plated at ˜20,000 cells per well of a 96 half-area well plate that contained drug. With both methods, cells were then grown in a humidified atmosphere with 5% ambient CO₂ for five days. Media was then removed from the plates and marker gene activity was measured by the addition of the β-galactosidase substrate o-nitrophenyl β-D-galactopyranoside (ONPG) in PBS with 0.5% NP-40. The resulting calorimetric reaction was measured using a spectrophotometric plate reader (Titertek Inc.) at 420 nm n All data was analyzed using the XLFit (IDBSm) computer program. pIC₅₀ represents the negative logarithm of the concentration of ligand that caused 50% inhibition of the constitutive receptor response. Percent inhibition was calculated as the difference between the absorbance measurements in the absence of added ligand compared with that in the presence of saturating concentrations of ligand normalized to the absorbance difference for the reference ligand (Substance P analog), which was assigned a value of 100%.

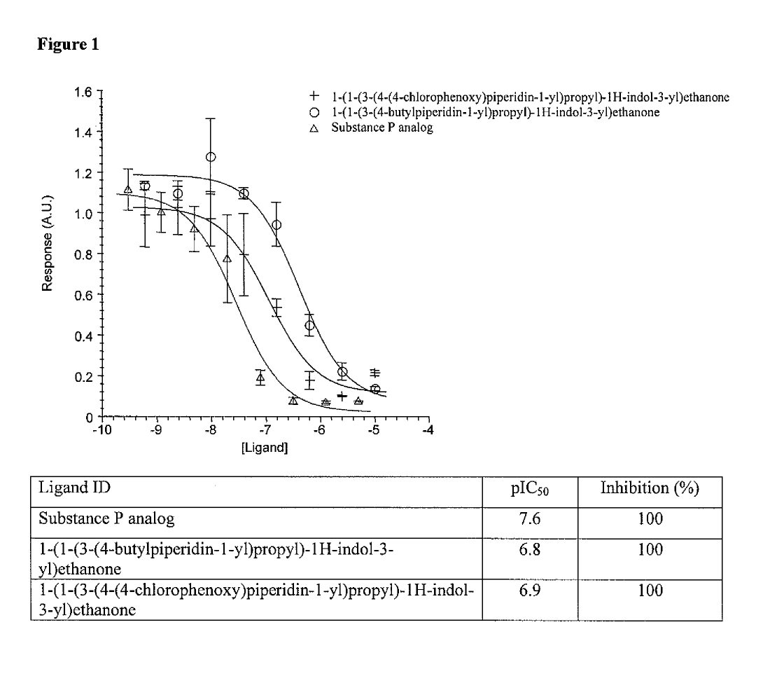

Data presented in FIG. 1 are derived from R-SAT assays as previously described (Jensen, A., A. et. al. (2000). J Biol Chem. 275(38): 29547-55). The concentration response relationship of the reference antagonist/inverse agonist Substance P analog (open triangles), 1-(1-(3-(4-(4-chlorophenoxy)piperidin-1-yl)propyl)-1H-indol-3-yl)ethanone (+signs) and 1-(1-(3-(4-butylpiperidin-1-yl)propyl)-1H-indol-3-yl)ethanone (open circles) to suppress constitutive activity of human Ghrelin receptors is shown. Data are plotted as response in absorbance units. Ligand activity is reported as −pIC₅₀, and percent inhibition relative to the reference antagonist/inverse agonist Substance P analog. Compounds of Formula I displayed potent ghrelin receptor antagonist/inverse agonist activity as disclosed in FIG. 1 and Appendix A. These compounds are ghrelin receptor antagonists/inverse agonists in this system.

Phosphatidyl Inositol Hydrolysis Assays (PI Assays)

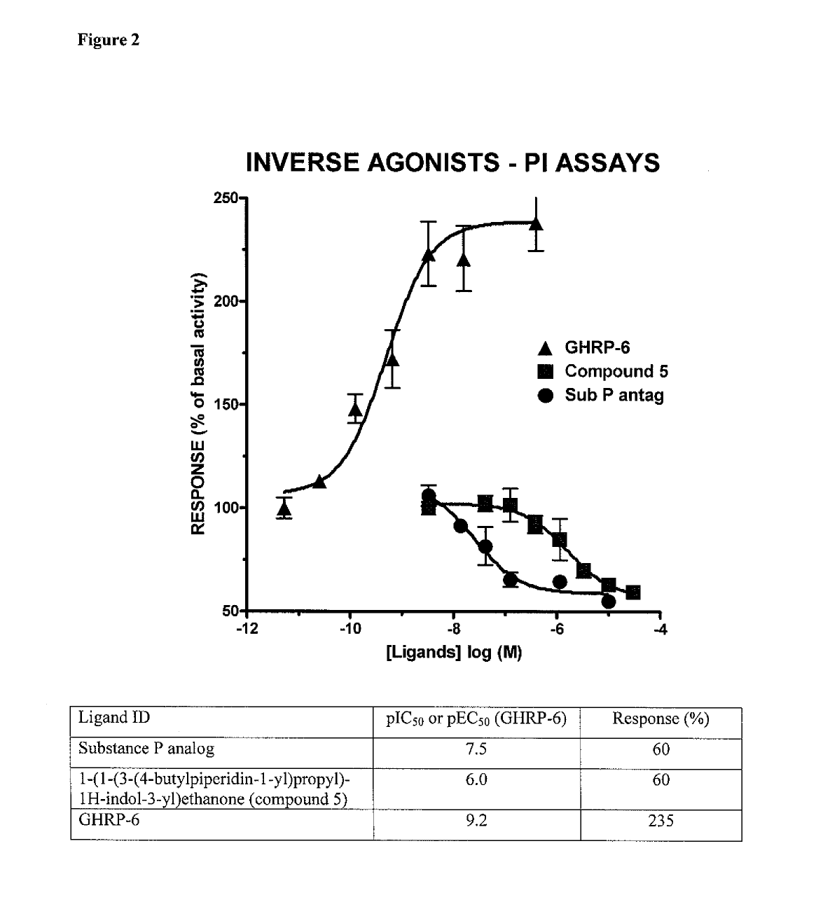

To confirm the observation that these compounds display ghrelin receptor inverse agonist activity, a PI hydrolysis assay was performed, the results of which are disclosed in FIG. 2. Data presented in FIG. 2 are derived from PI assays performed as described in (Jensen, A. A. et. al. (2000). J Biol Chem. 275(38): 29547-55). The concentration response relationship of the reference agonist GHRP-6 (filled triangles), the reference antagonist/inverse agonist Substance P analog (filled circles) and 1-(1-(3-(4-butylpiperidin-1-yl)propyl)-1H-indol-3-yl)ethanone (filled squares) to modulate the activity of human ghrelin receptors is shown. Data are plotted as the normalized response to basal activity (receptor activity in the absence of added ligands) versus drug concentration. Potency is reported as −pEC₅₀ values for GHRP-6, −pIC₅₀ values for substance P analog and 1-(1-(3-(4-butylpiperidin-1-yl)propyl)-1H-indol-3-yl)ethanone; and response is reported as that relative to the basal response in the absence of added ligands. Thus, two distinct in vitro functional assays confirm that analogs of Formula I possess potent antagonist/inverse agonist activity at human ghrelin receptors.

Suppression of Acute Feeding Response in Fasted Rats

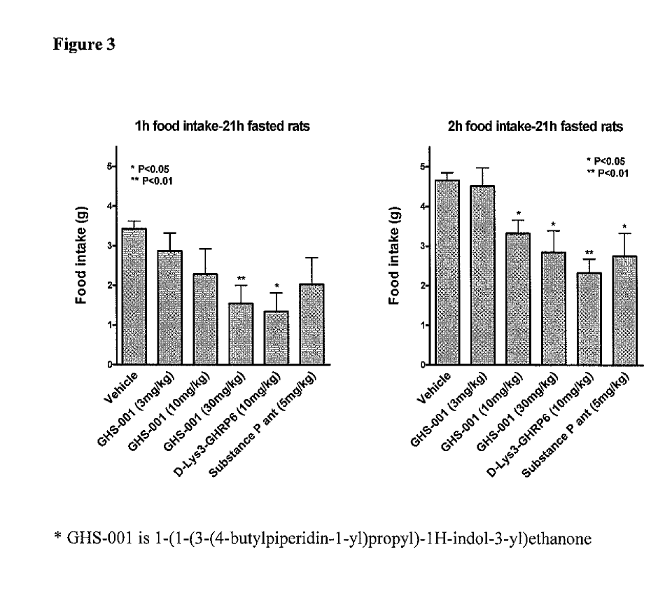

To confirm aspects of this molecular profile in vivo, 1-(1-(3-(4-butylpiperidin-1-yl)propyl)-1H-indol-3-yl)ethanone, the reference antagonist/inverse agonist Substance P analog, and the reference antagonist D-Lys3-GHRP-6 were administered intraperitoneally to rats, and the ghrelin receptor mediated stimulation of feeding was determined essentially as described previously (Sartor O, et al. Endocrinology. 1985 October; 117(4):1441-7), and this is disclosed in FIG. 3. Robust suppression of feeding was observed, at a dose of 30 mg/kg. This confirms that 1-(1-(3-(4-butylpiperidin-1-yl)propyl)-1H-indol-3-yl)ethanone functions as a ghrelin receptor antagonist in-vivo.

¹²⁵I-Ghrelin Binding Assay

To confirm the observation that these compounds bind to the ghrelin receptor, and can antagonize or block the binding of the endogenous GHSR1a agonist ghrelin, a binding assay was performed, the results of which are disclosed in Appendix B. Data presented in Appendix B is derived from binding assays performed as follows: 15 cm dishes were seeded with 4 million HEK293 cells in 16 ml 10% FCS/1% PSG/DMEM for transfection the next day. Plasmid DNA containing the GHSR1a receptor (12.5 ug/dish) in 0.675 ml DMEM was transfected into the cells by mixing with 180 ul PolyFect, 15 min later mixing in 2.25 ml 10% CS/DMEM, and transferring the mixture into the dish. At 16-18 h post transfection, medium was replaced with 25 ml fresh 10% FCS/1% PSG/DMEM to each dish for another 18-20 h. At approximately 48 hours post-transfection, cells were harvested in ice-cold membrane buffer (20 mM HEPES, 6 mM MgCl2, 1 mM EDTA, pH to 7.2) using a cell scraper, and pelleted by centrifugation.

Pelleted cells were added to a nitrogen cavitation chamber and 900 bar of pressure applied for 30 min. The pressure was slowly released the cavitated cells collected in 50 ml falcon tubes. The tubes were centrifuged at 1000 rpm, 4° C. for 10 min, and the supernatant collected. This centrifugation and collection was repeated two more times until the supernatant was free of precipitate (membranes are still in suspension). The supernatant was poured into a 50 ml centrifuge tube and centrifuged at 10.000 rpm, 4° C. for 20 min. The supernatant was discarded and the pellet re-suspended in 750 μl membrane binding buffer using a chilled 1 ml syringe with 25G5/8 needle to re-suspend membranes. The protein concentration was determined using the BioRad Protein Assay Dye Reagent according to the manufacturers instructions. The protein concentration was adjusted to 5 mg/ml and aliquots snap-frozed and stored at −80 C until use.

Membranes were thawed rapidly; diluted with binding buffer (25 mM Hepes+5 mM MgCl2, 1 mM CaCl₂, 2.5 mM EDTA and 0.2% BSA) to a protein concentration of 0.8 ug/30 ul and placed on ice. 96-well plates (U-bottom wells) were prepared with serial dilutions (8 doses, 40 ul/w) of the test compounds. Membranes (30 uL/well) were then added, and incubated with test ligands for 30 min at room temperature with shaking. 30 ul ¹²⁵I-ghrelin (0.053 nM) was then added to each well, and the plates incubated for another 2.5 hours with shaking. Binding was terminated by filtration through GF/B filters (presoaked with 0.1% polyethylenimine) with a Brandel 96-well harvester. The filters were washed with ice-cold binding buffer (150 ml/plate) and allowed to air-dry for 30 min. 50 ul MicroScient-20 cocktail was added to each dried well, the plates were sealed, and counted for 2 min/well using a TopCount scintillation counter (Packard).

All data was analyzed using the Prizm computer program. pKi represents the negative logarithm of the concentration of ligand that caused 50% displacement of bound ¹²⁵I-ghrelin adjusted for the concentration of radioligand using the Cheng-Prussoff equation: Ki=IC₅₀/{1+[test ligand]/Kd_{ghrelin}} to derive Ki values. Percent inhibition was calculated as the difference between the bound ¹²⁵I-ghrelin in the absence of added ligand compared with that in the presence of saturating concentrations of ligand normalized to the absorbance difference for the reference ligand (ghrelin), which was assigned a value of 100% (not shown).

Although the invention has been described with reference to the above examples, it will be understood that modifications and variations are encompassed within the spirit and scope of the invention. Accordingly, the invention is limited only by the following claims.

| | RSAT | | Binding | Aeg |
|---|---|---|---|---|
| Ligand | pIC50 | Eff(%) | pKi | pIC50 |
| D-Lys3-GHRP6 | nd | | 6.6 | |
| Ghrelin | nd | | 10.2 | |
| substance P analog | 7.4 | 100 | 7.7 | 8.5 |
| 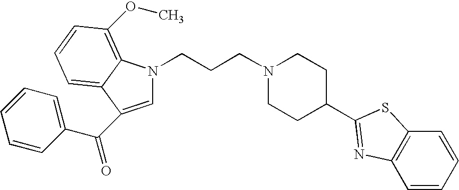 | 9.7 | 107 | 7.4 | 7.2 |
| 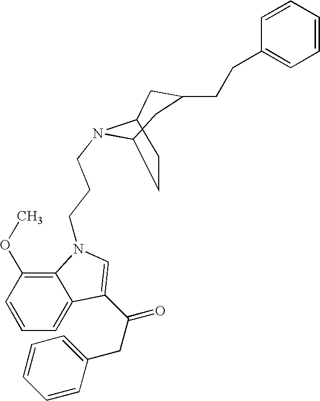 | 9.6 | 141 | 8.0 | 6.9 |
| 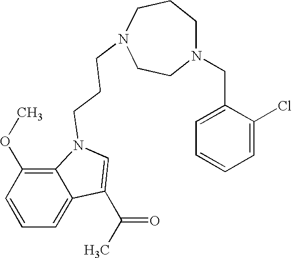 | 9.6 | 128 | 8.0 | 6.9 |
| 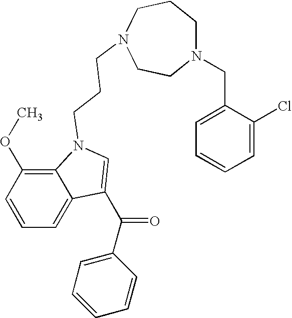 | 9.4 | 98 | 8.4 | 6.5 |
| 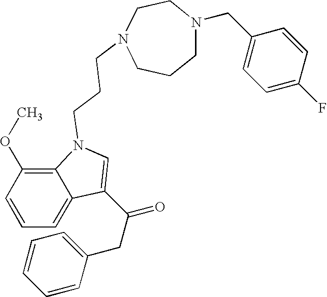 | 9.4 | 87 | 8.1 | |
| 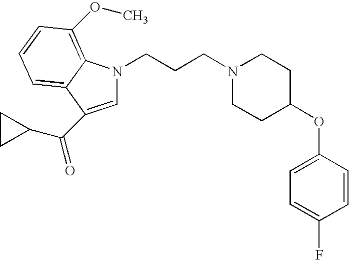 | 9.4 | 101 | 7.3 | 74 |
| 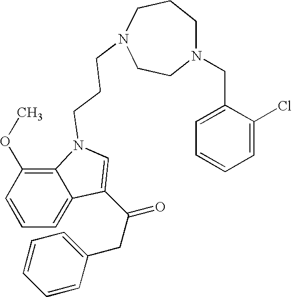 | 9.3 | 92 | 8.2 | 7.2 |
| 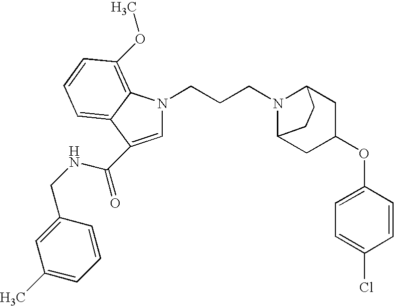 | 9.2 | 92 | 7.3 | 6.8 |
| 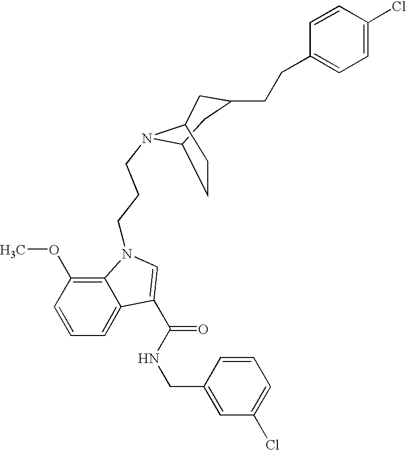 | 9.1 | 127 | | |
| 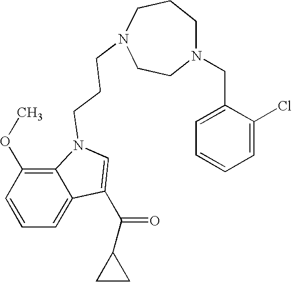 | 9.1 | 99 | 8.1 | 6.6 |
| 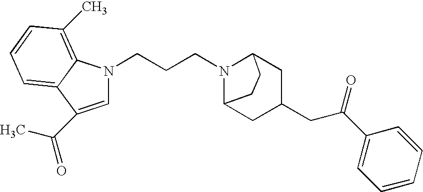 | 9.1 | 91 | 6.7 | |
| 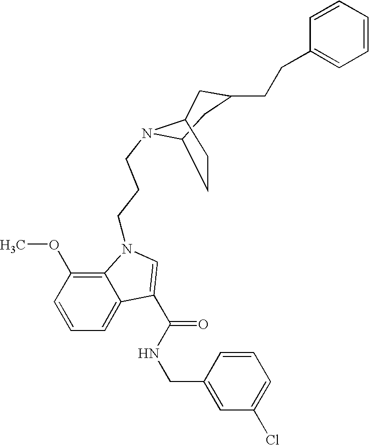 | 9.0 | 115 | 8.1 | 5.9 |
| 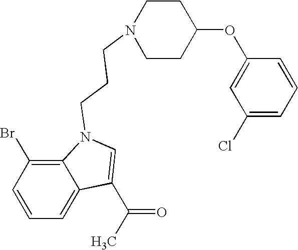 | 9.0 | 92 | | |
| 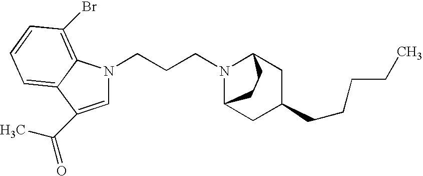 | 8.9 | 81 | 6.8 | |
| 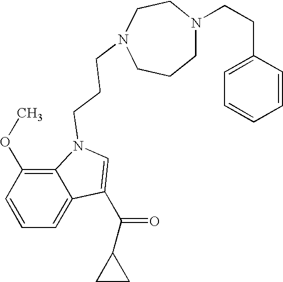 | 8.9 | 100 | | |
| 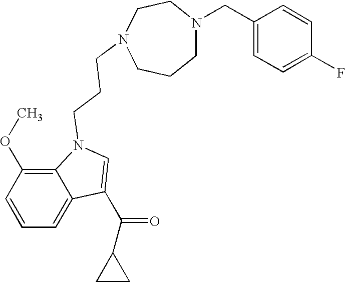 | 8.9 | 92 | 8.1 | 7.9 |
| 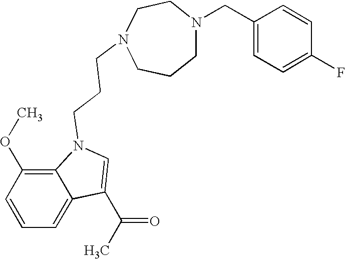 | 8.9 | 130 | 7.8 | |
| 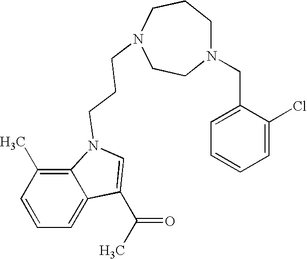 | 8.9 | 111 | | |
| 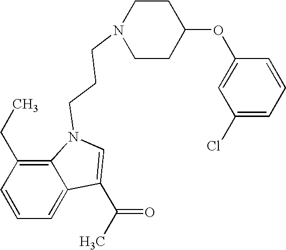 | 8.8 | 118 | 7.5 | 6.9 |
| 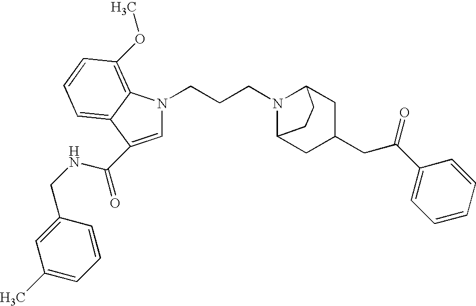 | 8.8 | 106 | 8.7 | 7.4 |
| 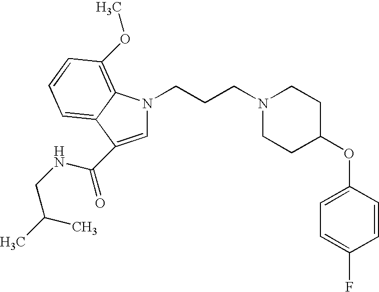 | 8.8 | 90 | 6.9 | |
| 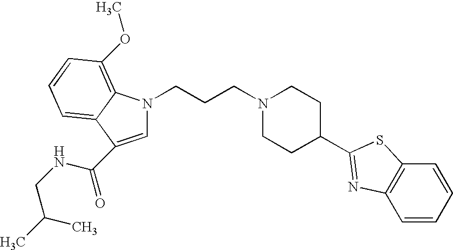 | 8.8 | 106 | 6.0 | |
| 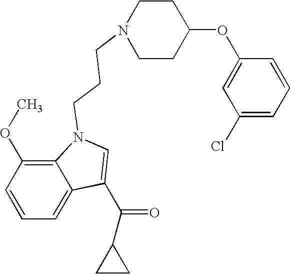 | 8.8 | 102 | 7.7 | 6.3 |
| 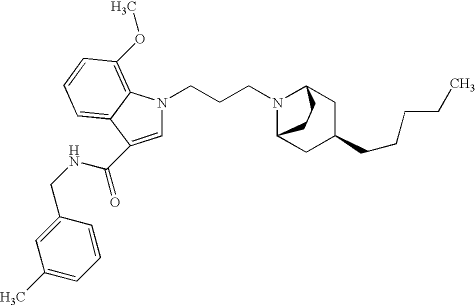 | 8.7 | 90 | 7.3 | 6.8 |
| 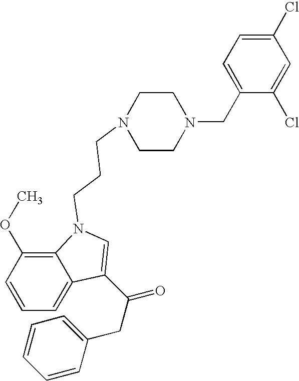 | 8.7 | 76 | | |
| 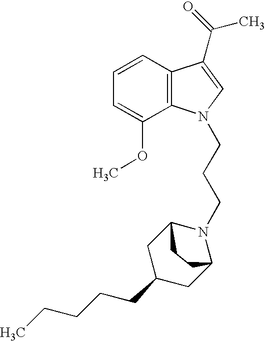 | 8.7 | 113 | 6.5 | |
| 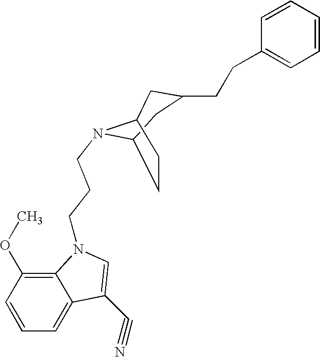 | 8.7 | 104 | 7.6 | |
| 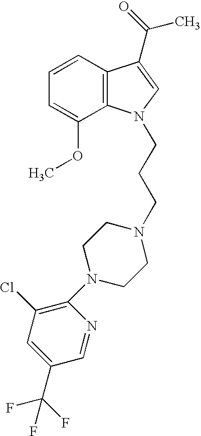 | 8.6 | 103 | 6.4 | |
| 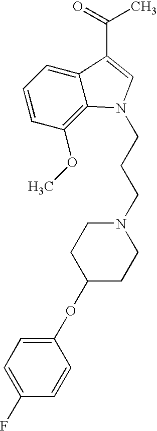 | 8.6 | 104 | 6.1 | |
| 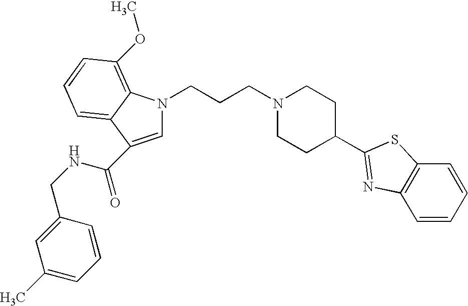 | 8.6 | 94 | 7.2 | |
| 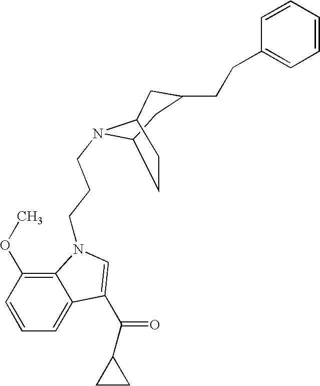 | 8.6 | 109 | | |
| 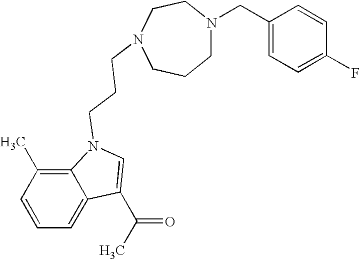 | 8.6 | 95 | | |
| 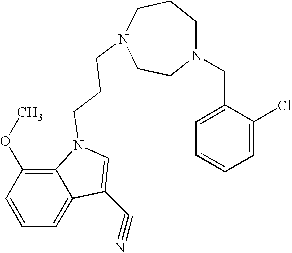 | 8.6 | 137 | 7.9 | |
| 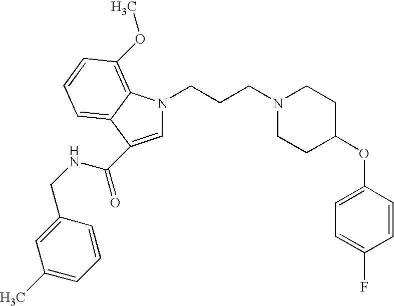 | 8.5 | 81 | 7.0 | 6.3 |
| 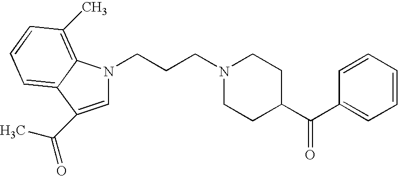 | 8.5 | 96 | 7.0 | |
| 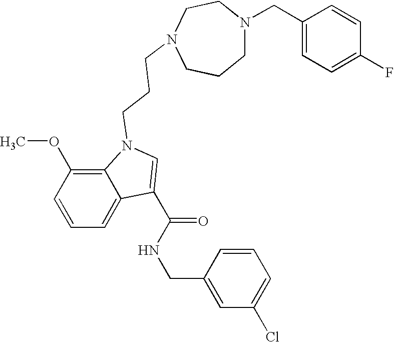 | 8.5 | 113 | — | |
| 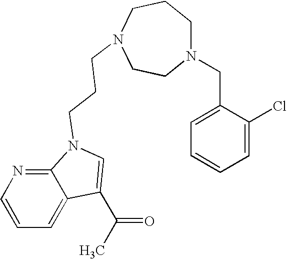 | 8.5 | 115 | 7.6 | 7.6 |
| 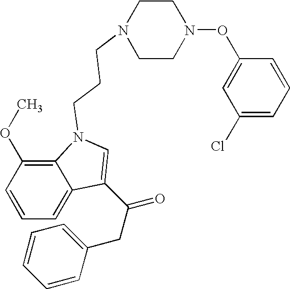 | 8.5 | 88 | 7.8 | |
| 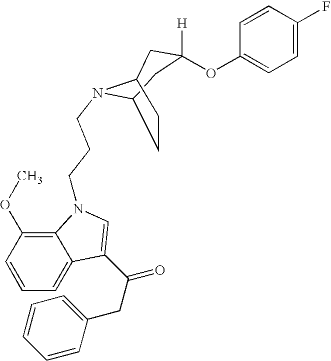 | 8.4 | 99 | 6.6 | |
| 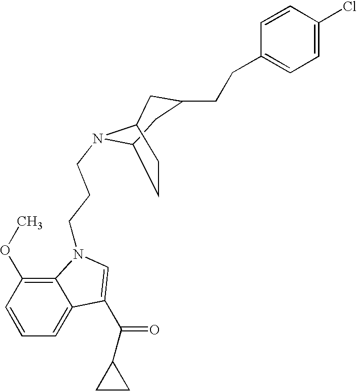 | 8.4 | 103 | 7.7 | |
| 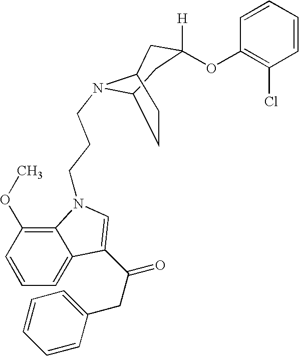 | 8.4 | 129 | — | |
| 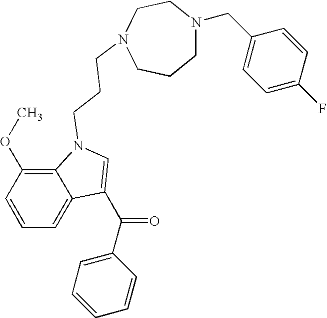 | 8.4 | 96 | 8.0 | 7.5 |
| 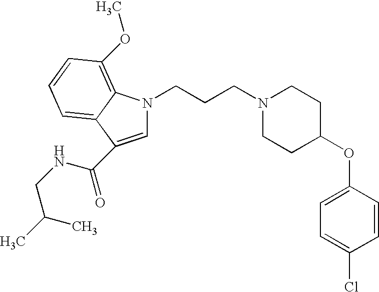 | 8.4 | 146 | 7.3 | 7.0 |
| 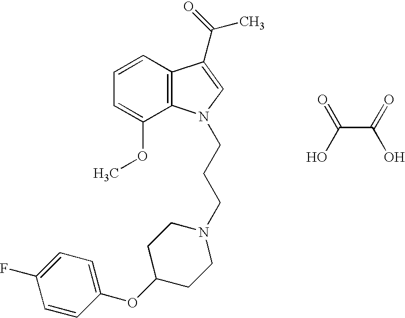 | 8.4 | 119 | 6.8 | |
| 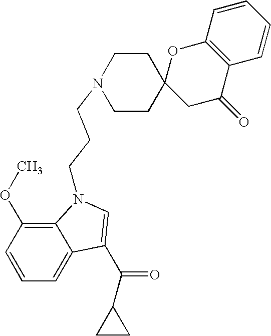 | 8.4 | 106 | | |
| 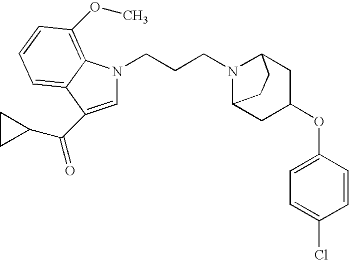 | 8.4 | 86 | 6.8 | |
| 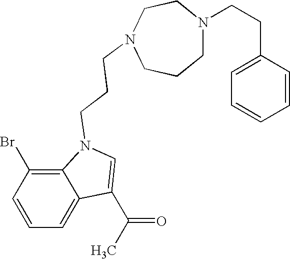 | 8.4 | 98 | 7.1 | |
| 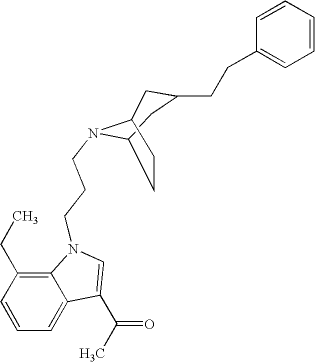 | 8.4 | 103 | 6.9 | |
| 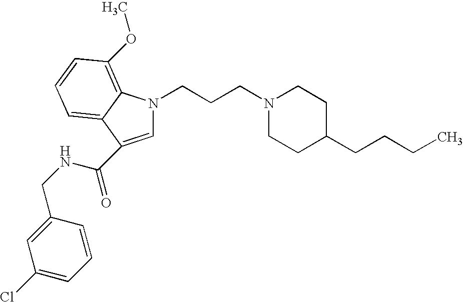 | 8.4 | 94 | 7.3 | |
| 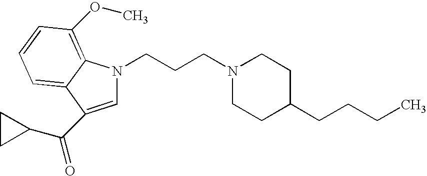 | 8.4 | 95 | 6.4 | |
| 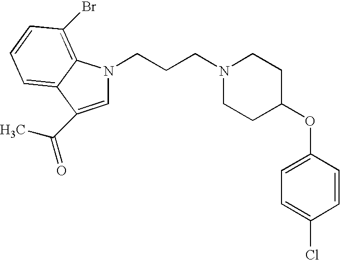 | 8.4 | 105 | 7.0 | 6.5 |
| 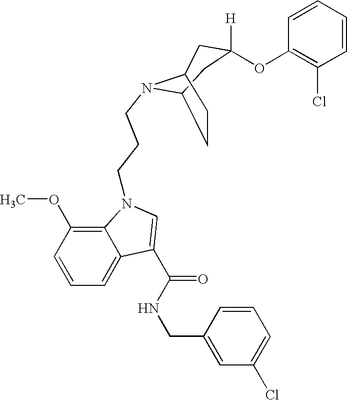 | 8.4 | 99 | 7.7 | |
| 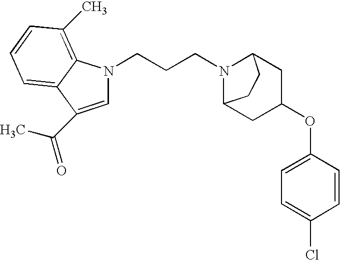 | 8.3 | 99 | 6.9 | |
| 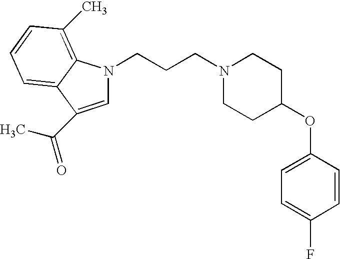 | 8.3 | 72 | 6.7 | |
| 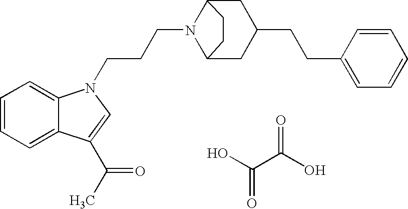 | 8.3 | 105 | 6.5 | |
| 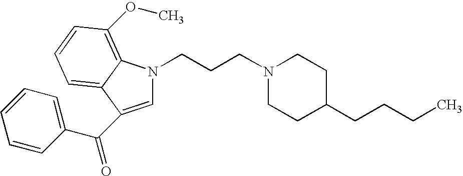 | 8.3 | 98 | 6.7 | |
| 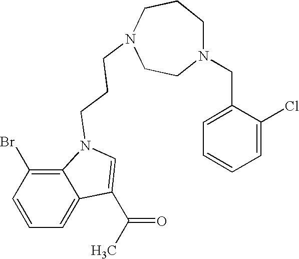 | 8.3 | 95 | 8.0 | |
| 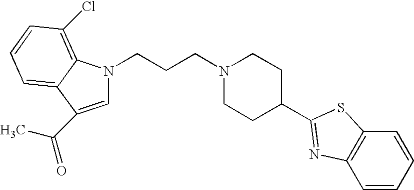 | 8.3 | 117 | 6.4 | |
| 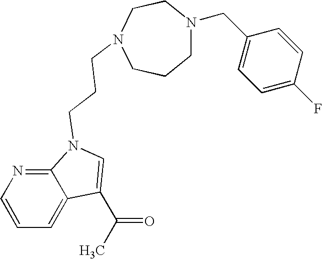 | 8.3 | 136 | | |
| 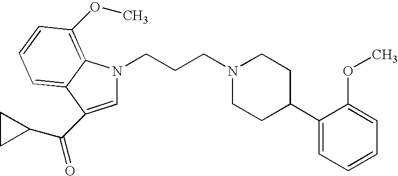 | 8.3 | 102 | 6.8 | |
| 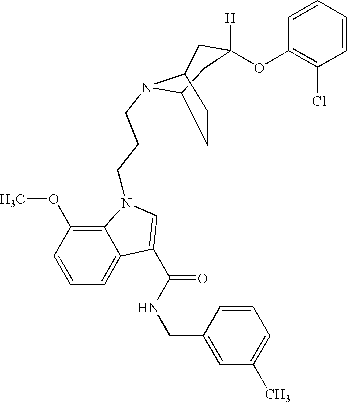 | 8.3 | 121 | 7.7 | |
| 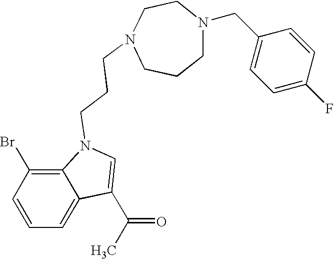 | 8.3 | 95 | | |
| 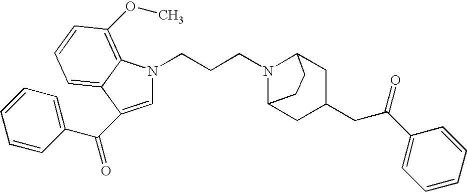 | 8.3 | 101 | 7.0 | |
| 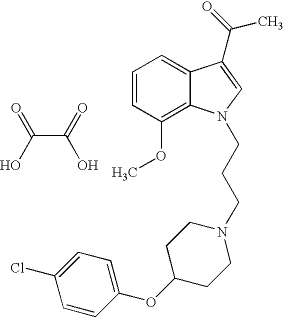 | 8.3 | 144 | 6.7 | |
| 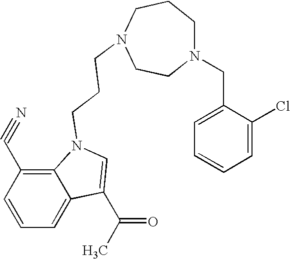 | 8.2 | 98 | 7.2 | |
| 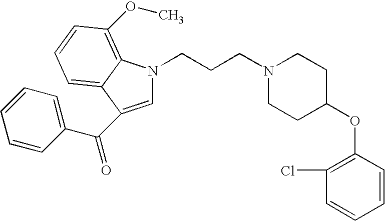 | 8.2 | 79 | 6.8 | |
| 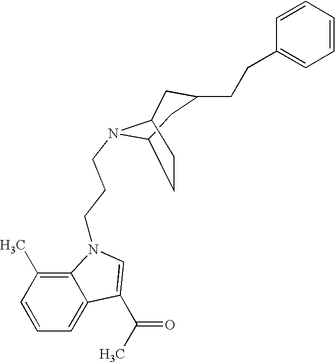 | 8.2 | 105 | | |
| 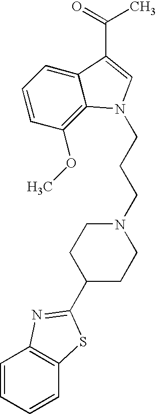 | 8.2 | 128 | 6.4 | |
| 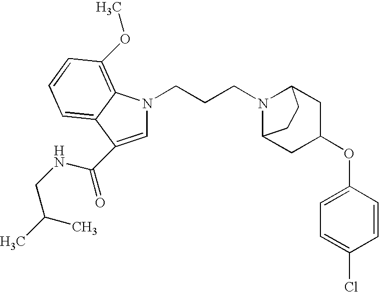 | 8.2 | 107 | 7.0 | 6.6 |
| 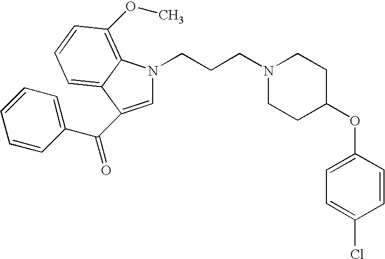 | 8.2 | 93 | 7.0 | 6.6 |
| 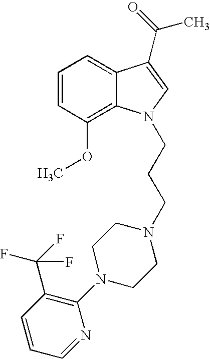 | 8.2 | 108 | 6.0 | |
| 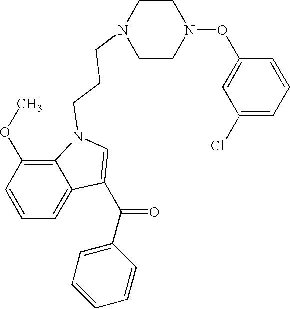 | 8.2 | 79 | 8.1 | |
| 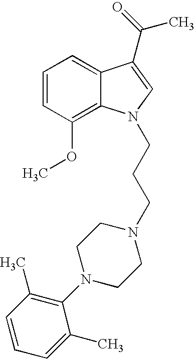 | 8.2 | 107 | 6.0 | |
| 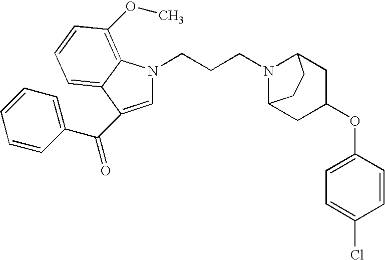 | 8.1 | 91 | 7.0 | 6.2 |
| 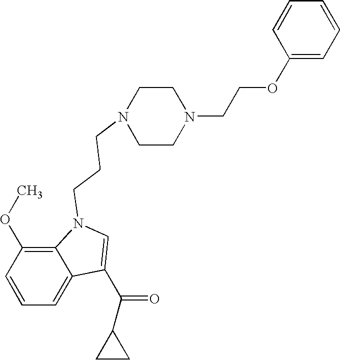 | 8.1 | 94 | | |
| 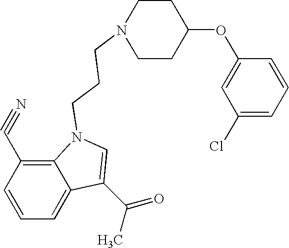 | 8.1 | 101 | 6.8 | |
| 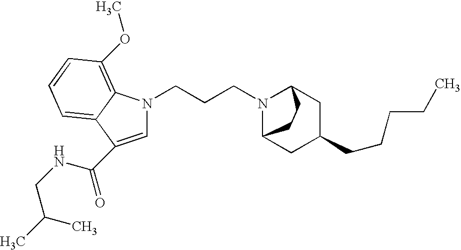 | 8.1 | 135 | 7.1 | 6.5 |
| 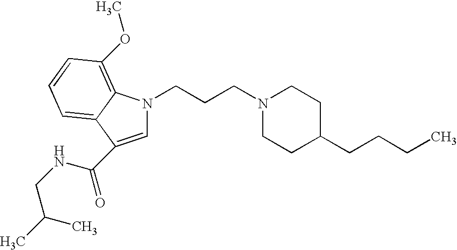 | 8.1 | 93 | 6.7 | |
| 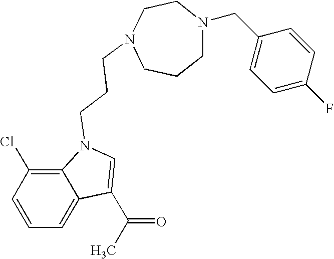 | 8.1 | 115 | | |
| 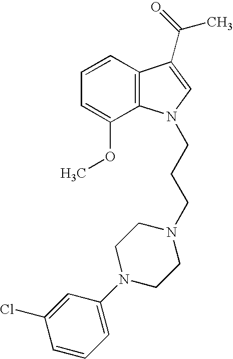 | 8.1 | 99 | 6.3 | |
| 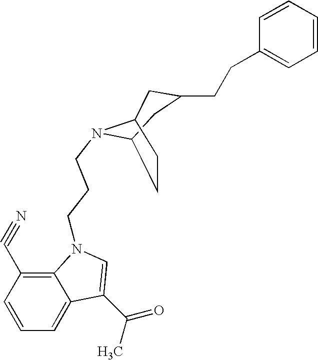 | 8.1 | 106 | 7.4 | |
| 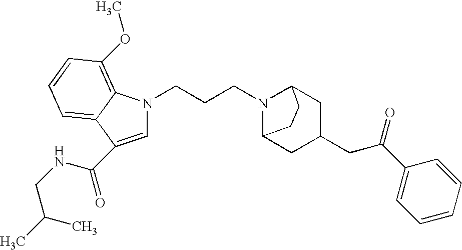 | 8.1 | 145 | 7.3 | 6.8 |
| 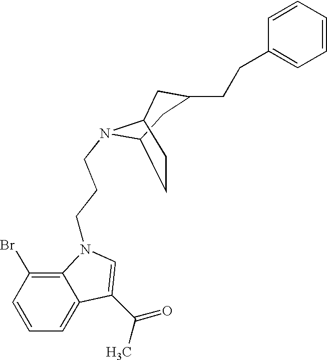 | 8.1 | 107 | 7.1 | |
| 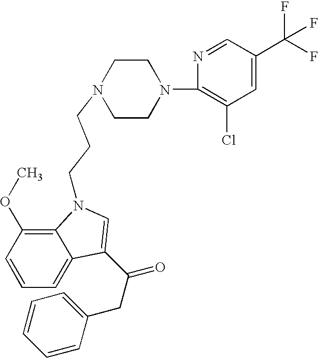 | 8.1 | 95 | | |
| 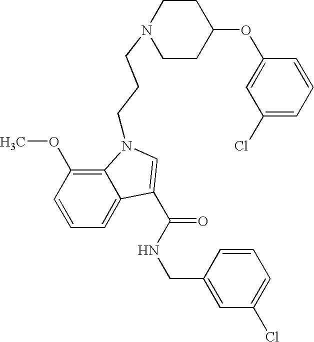 | 8.1 | 100 | 7.2 | |
| 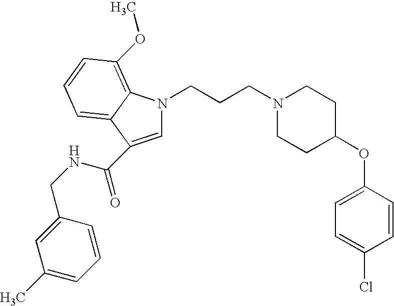 | 8.1 | 115 | 6.9 | |
| 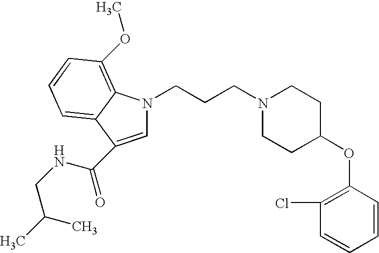 | 8.1 | 100 | 7.2 | 7.0 |
| 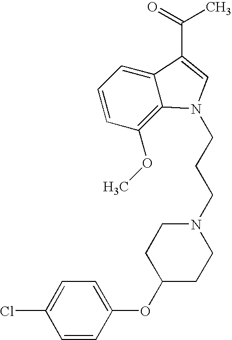 | 8.1 | 129 | 6.9 | |
| 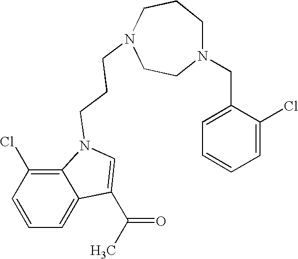 | 8.1 | 92 | 71 | |
| 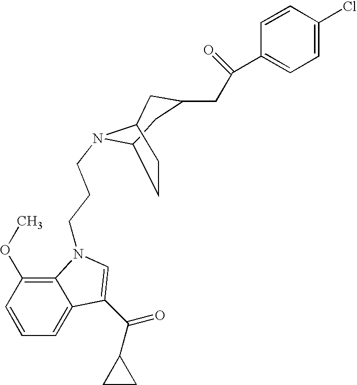 | 8.1 | 70 | 7.4 | |
| 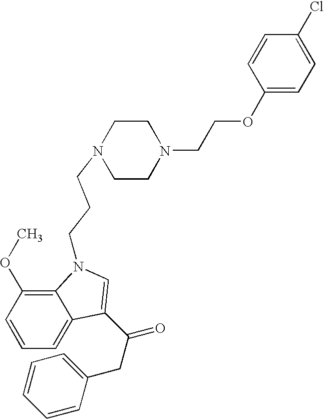 | 8.1 | 82 | 5.5 | |
| 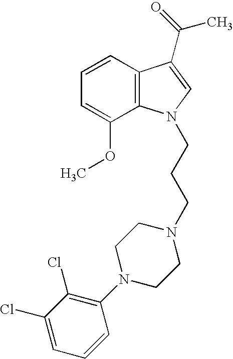 | 8.1 | 103 | 6.4 | |
| 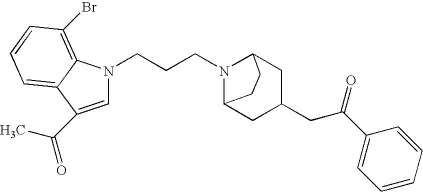 | 8.1 | 111 | 7.0 | |
| 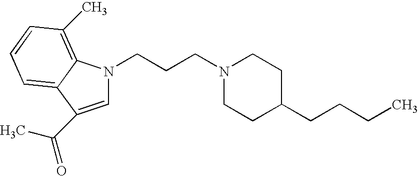 | 8.1 | 78 | 6.5 | |
| 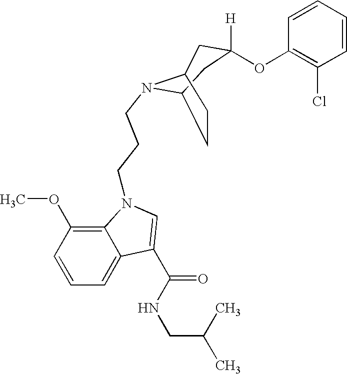 | 8.0 | 116 | 7.0 | |
| 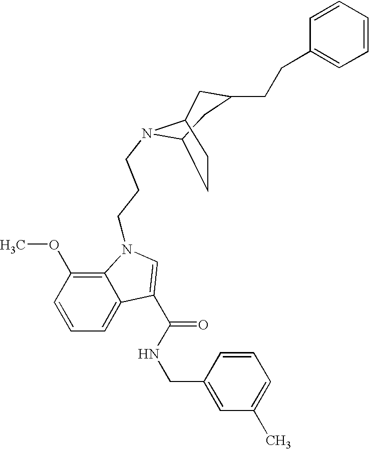 | 8.0 | 89 | | |
| 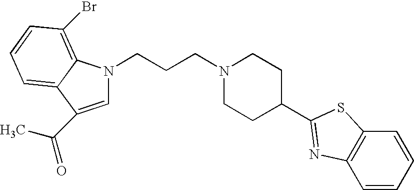 | 8.0 | 81 | 6.7 | |
| 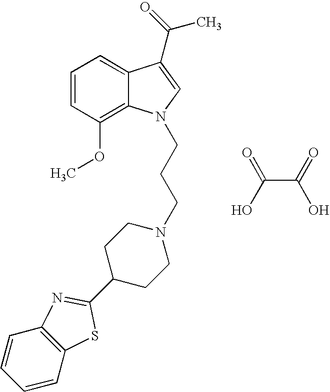 | 8.0 | 102 | 6.8 | |
| 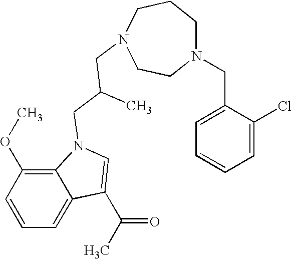 | 8.0 | 123 | 7.0 | |
| 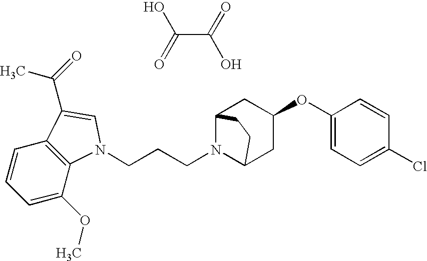 | 8.0 | 114 | 6.7 | |
| 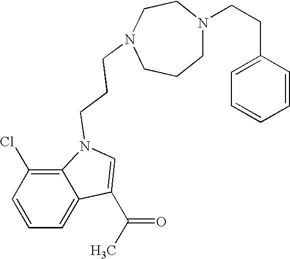 | 8.0 | 96 | | |
| 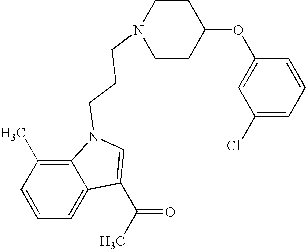 | 8.0 | 96 | | |
| 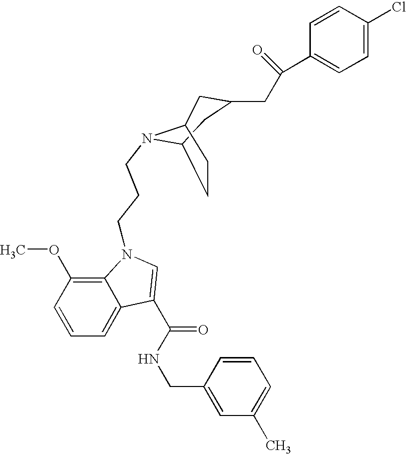 | 8.0 | 110 | 7.8 | |
| 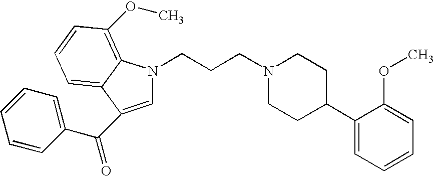 | 7.9 | 88 | 6.8 | |
| 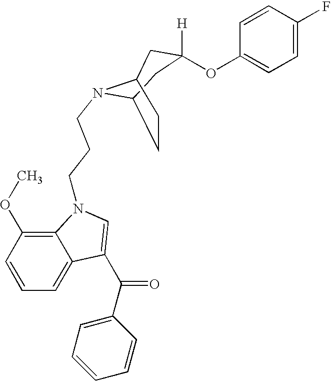 | 7.9 | 106 | 6.8 | |
| 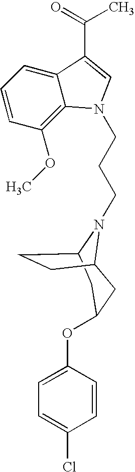 | 7.9 | 113 | 6.3 | |
| 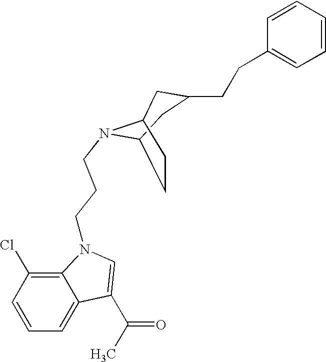 | 7.9 | 109 | | |
| 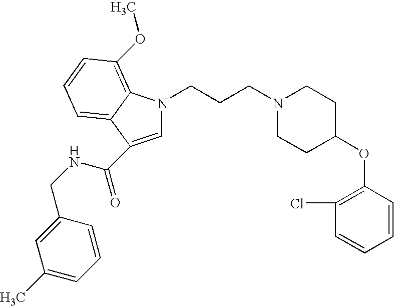 | 7.9 | 96 | 7.1 | 6.6 |
| 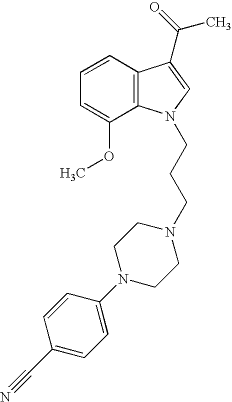 | 7.9 | 90 | 6.2 | |
| 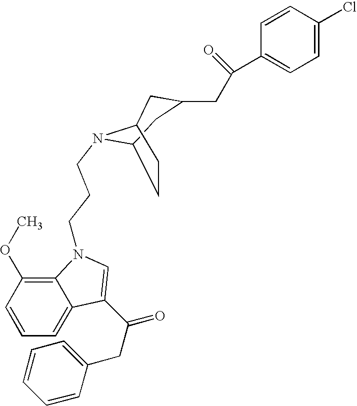 | 7.9 | 167 | 7.5 | |
| 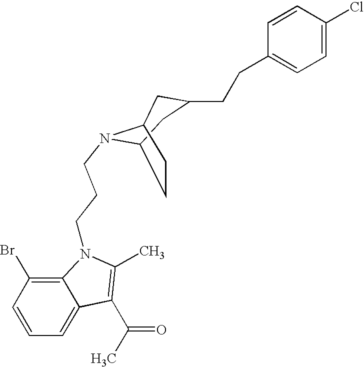 | 7.9 | 69 | 6.5 | |
| 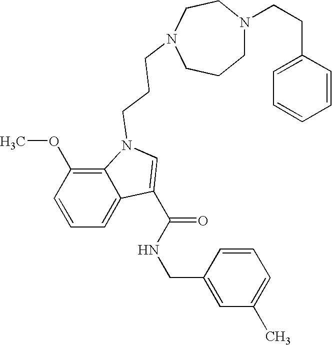 | 7.9 | 110 | 7.8 | |
| 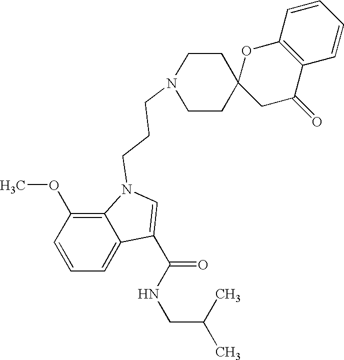 | 7.9 | 115 | | |
| 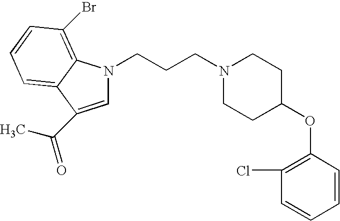 | 7.9 | 96 | 6.9 | |
| 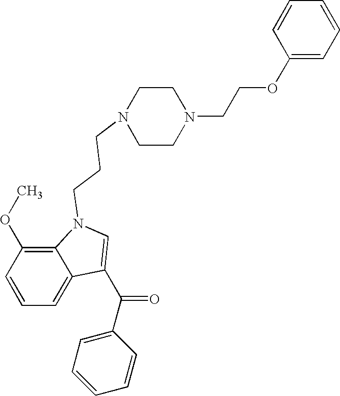 | 7.9 | 98 | 6.6 | |
| 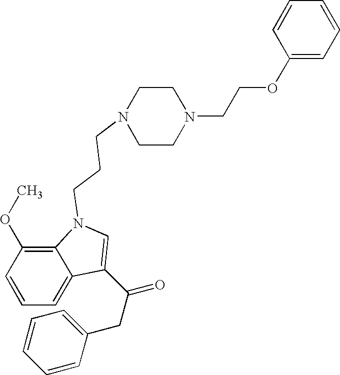 | 7.9 | 99 | 7.4 | |
| 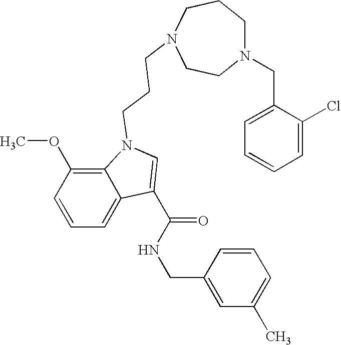 | 7.8 | 119 | 7.1 | |
| 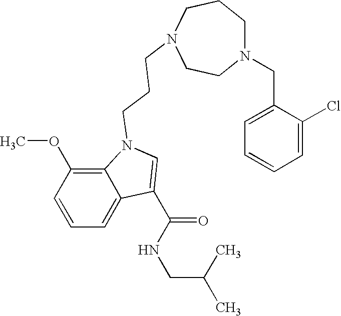 | 7.8 | 100 | | |
| 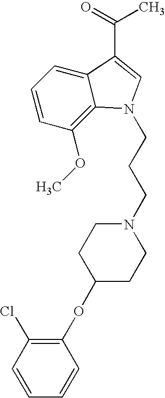 | 7.8 | 98 | 6.1 | |
| 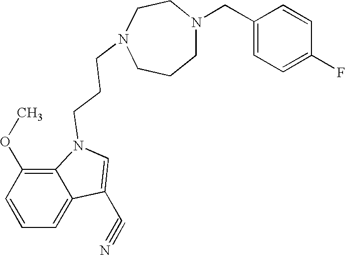 | 7.8 | 109 | — | |
| 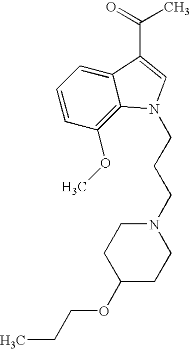 | 7.8 | 112 | 5.7 | |
| 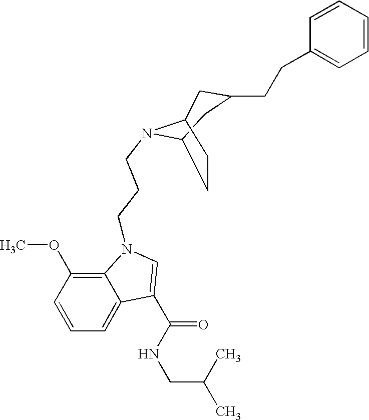 | 7.8 | 86 | | |
| 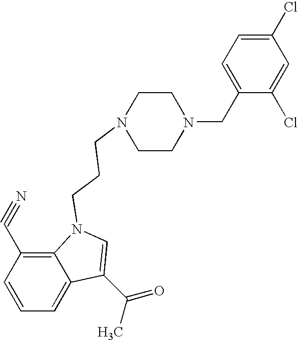 | 7.8 | 92 | | |
| 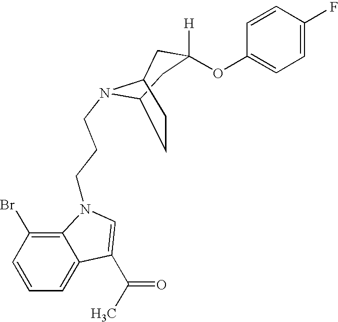 | 7.8 | 81 | | |
| 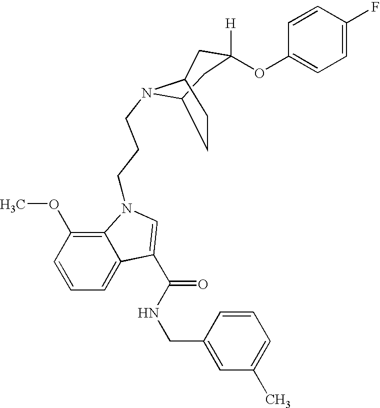 | 7.8 | 112 | | |
| 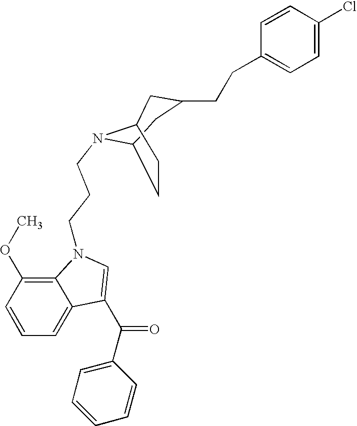 | 7.8 | 94 | | |
| 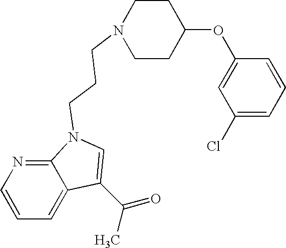 | 7.8 | 124 | 6.5 | |
| 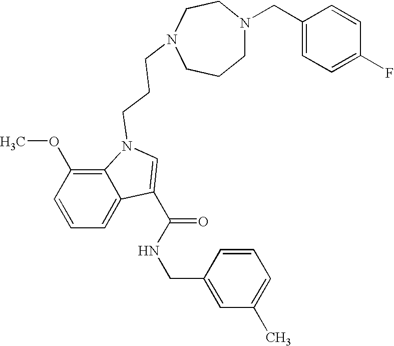 | 7.8 | 129 | 7.0 | |
| 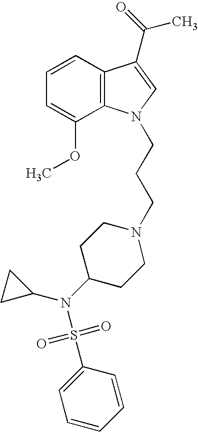 | 7.8 | 105 | 5.6 | |
| 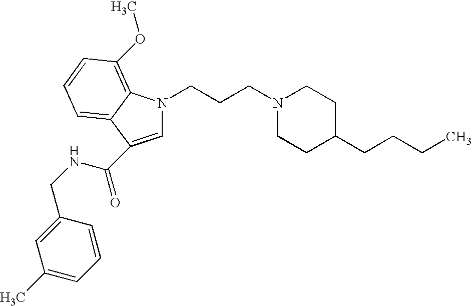 | 7.8 | 72 | 6.5 | |
| 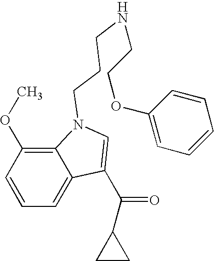 | 7.8 | 83 | | |
| 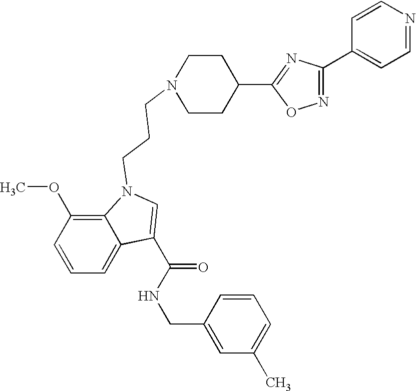 | 7.8 | 121 | — | |
| 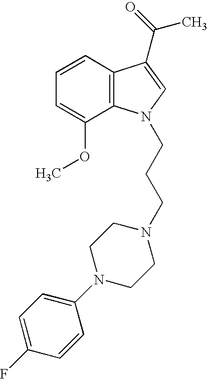 | 7.8 | 95 | 6.3 | |
| 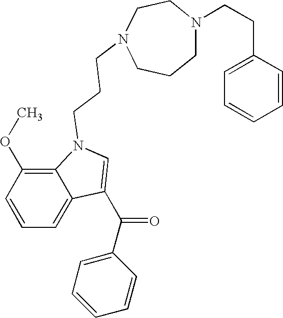 | 7.8 | 124 | 7.6 | |
| 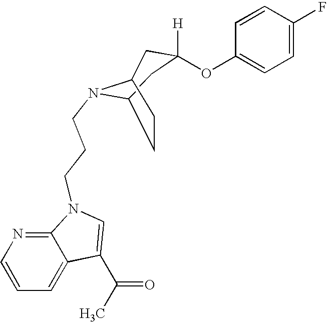 | 7.8 | 77 | 6.3 | |
| 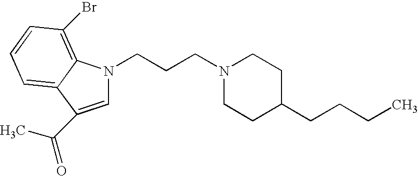 | 7.7 | 87 | 6.8 | |
| 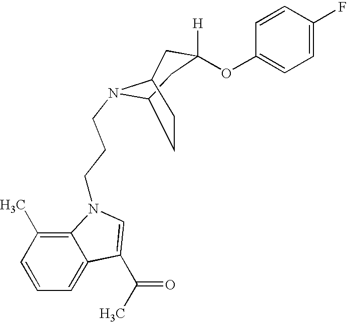 | 7.7 | 98 | | |
| 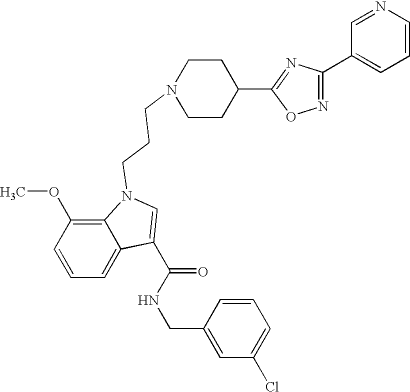 | 7.7 | 103 | | |
| 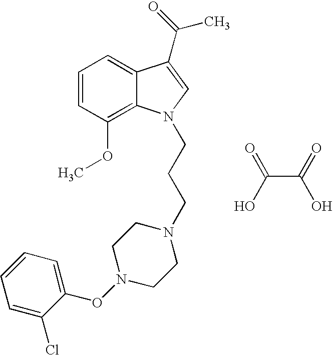 | 7.7 | 100 | 6.5 | |
| 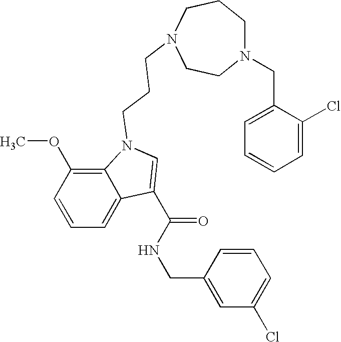 | 7.7 | 107 | | |
| 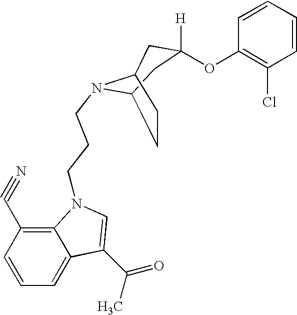 | 7.7 | 83 | 6.7 | |
| 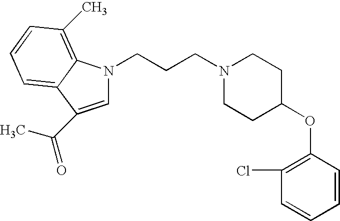 | 7.7 | 110 | 6.2 | |
| 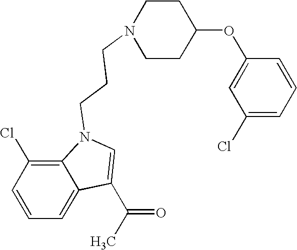 | 7.7 | 83 | 7.0 | |
| 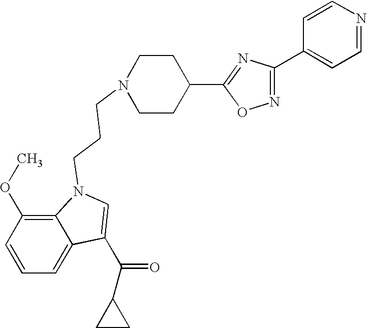 | 7.7 | 87 | | |
| 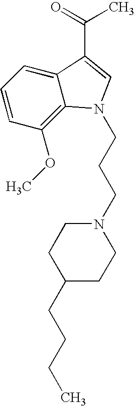 | 7.7 | 116 | 5.8 | |
| 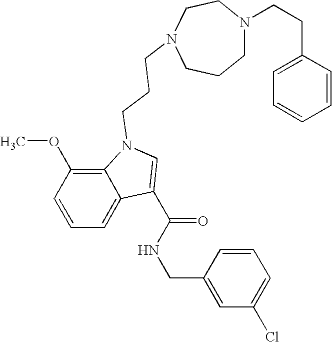 | 7.7 | 125 | 6.8 | |
| 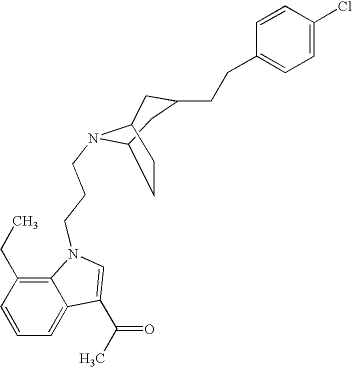 | 7.7 | 95 | 7.0 | |
| 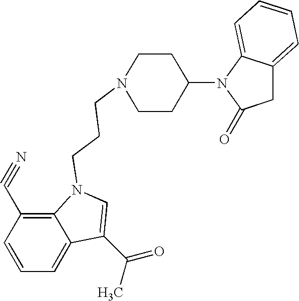 | 7.7 | 133 | | |
| 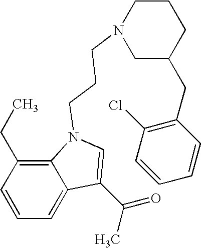 | 7.7 | 87 | — | |
| 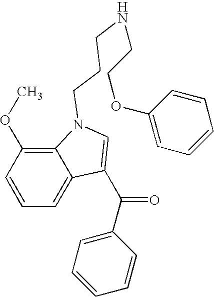 | 7.7 | 74 | 6.8 | |
| 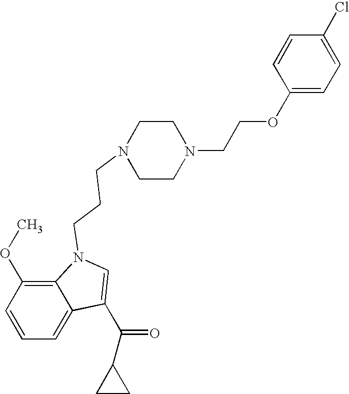 | 7.7 | 122 | 6.9 | |
| 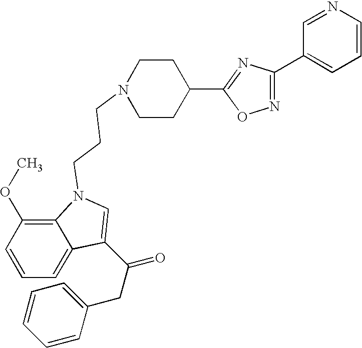 | 7.7 | 88 | | |
| 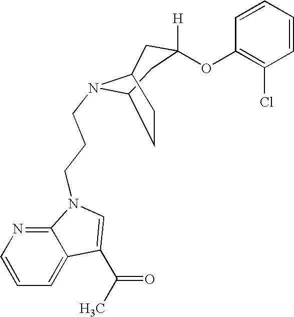 | 7.7 | 108 | 7.5 | |
| 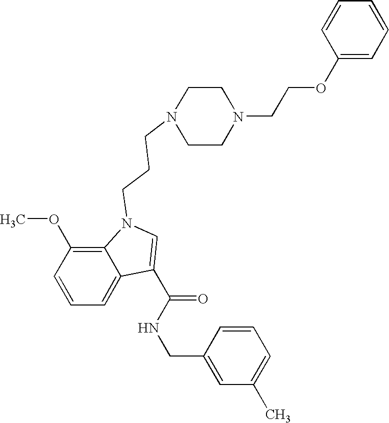 | 7.7 | 89 | 8.1 | 8.8 |
| 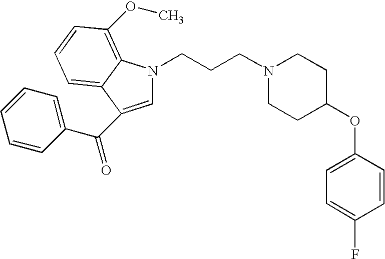 | 7.6 | 107 | 5.9 | |
| 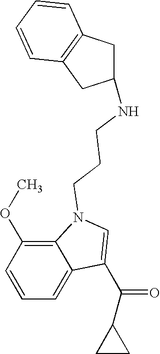 | 7.6 | 97 | | |
| 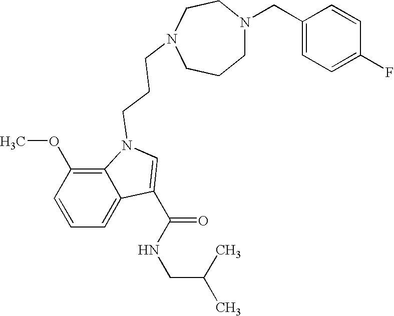 | 7.6 | 118 | 7.0 | |
| 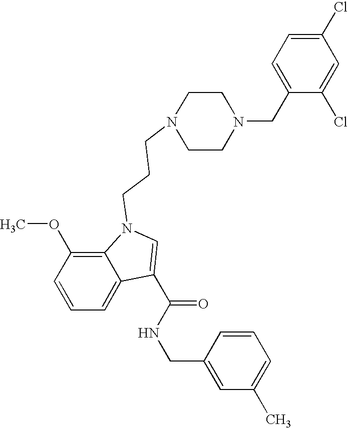 | 7.6 | 110 | 6.0 | |
| 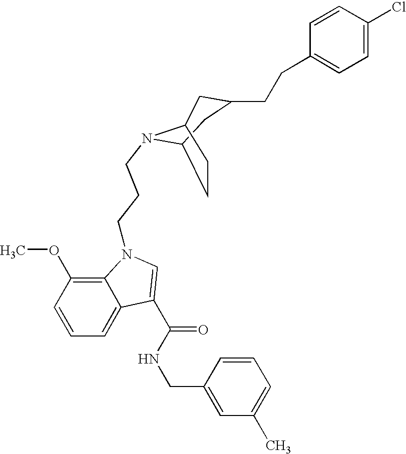 | 7.6 | 77 | | |
| 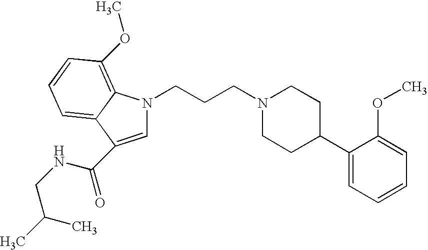 | 7.6 | 108 | 6.9 | |
| 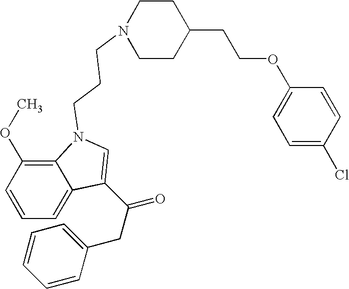 | 7.6 | 121 | 6.6 | |
| 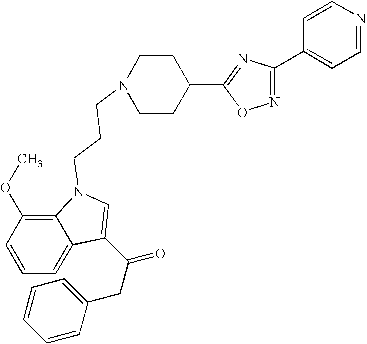 | 7.6 | 137 | | |
| 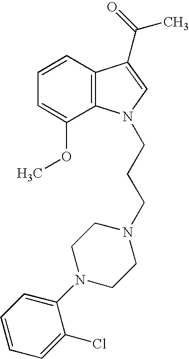 | 7.6 | 108 | 5.6 | |
| 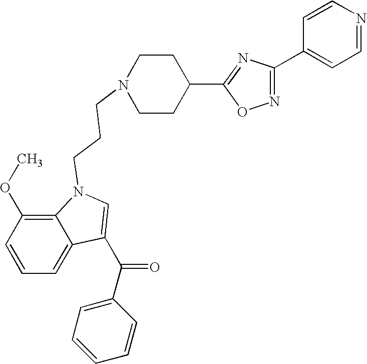 | 7.6 | 98 | — | |
| 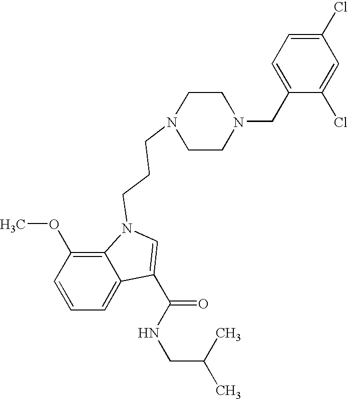 | 7.6 | 105 | | |
| 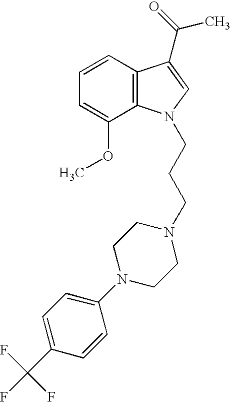 | 7.6 | 92 | 6.6 | |
| 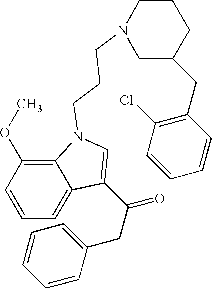 | 7.6 | 116 | 6.9 | |
| 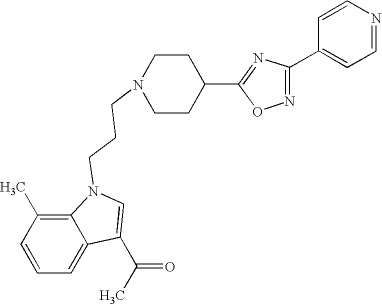 | 7.5 | 137 | | |
| 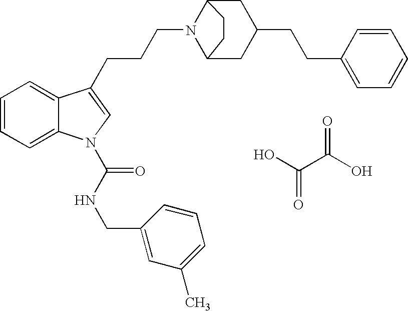 | 7.5 | 114 | 6.8 | |
| 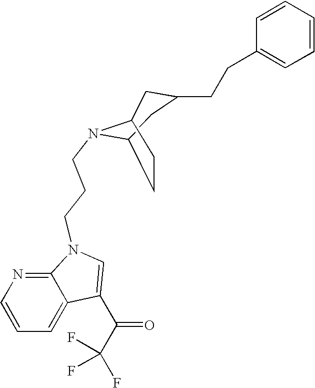 | 7.5 | 104 | 6.7 | |
| 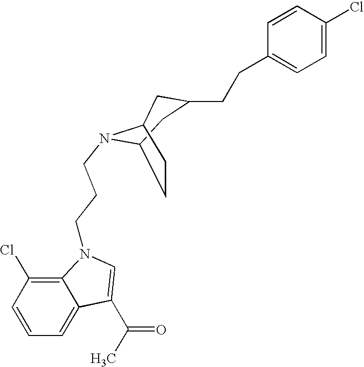 | 7.5 | 122 | | |
| 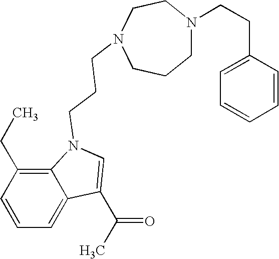 | 7.5 | 103 | — | |
| 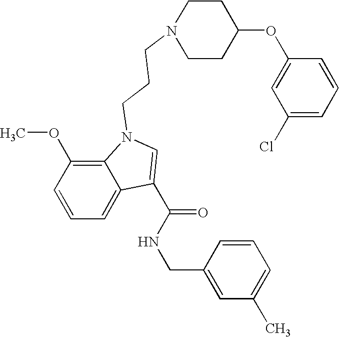 | 75 | 93 | | |
| 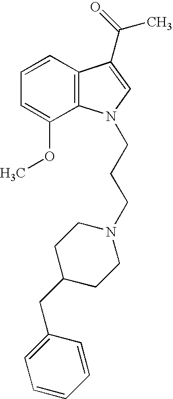 | 7.5 | 116 | 6.1 | |
| 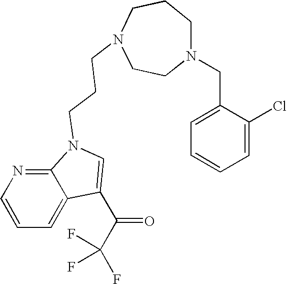 | 7.5 | 85 | 7.3 | |
| 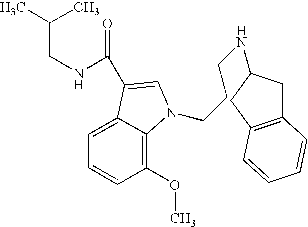 | 7.5 | 102 | | |
| 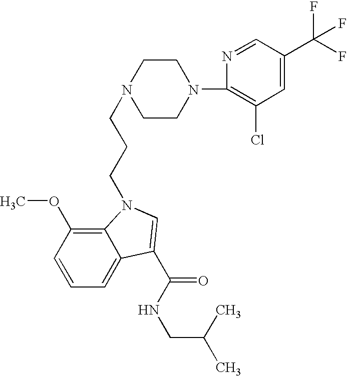 | 7.5 | 100 | — | |
| 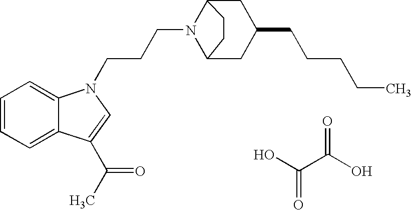 | 7.5 | 104 | 5.8 | |
| 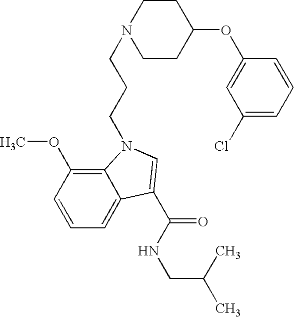 | 7.5 | 71 | | |
| 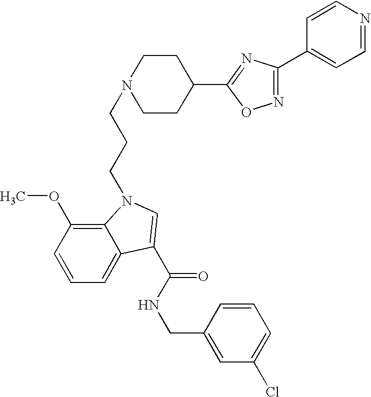 | 7.5 | 105 | — | |
| 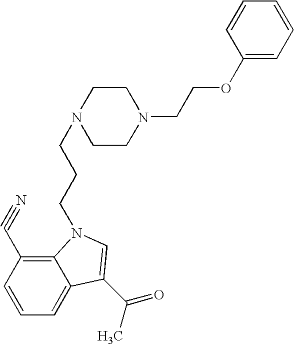 | 7.5 | 86 | | |
| 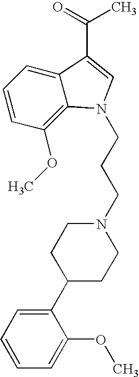 | 7.4 | 109 | 6.2 | |
| 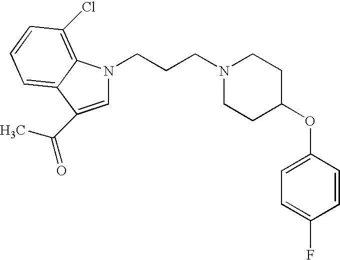 | 7.4 | 120 | 5.7 | |
| 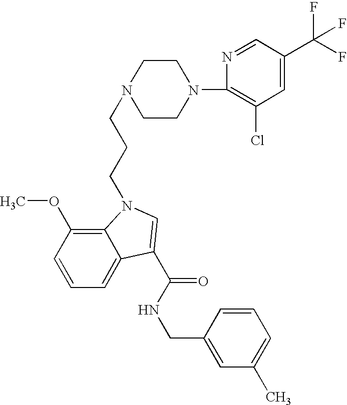 | 7.4 | 80 | | |
| 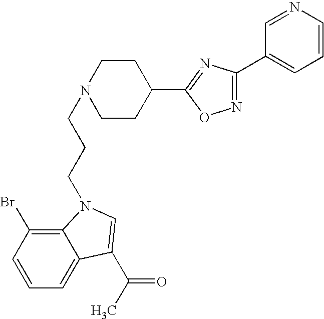 | 7.4 | 119 | | |
| 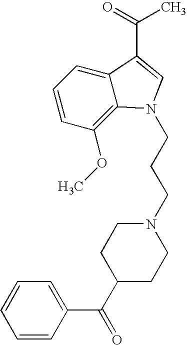 | 7.4 | 98 | 5.7 | |
| 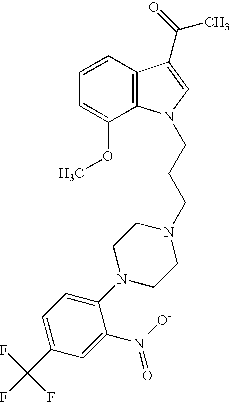 | 7.4 | 94 | 6.2 | |
| 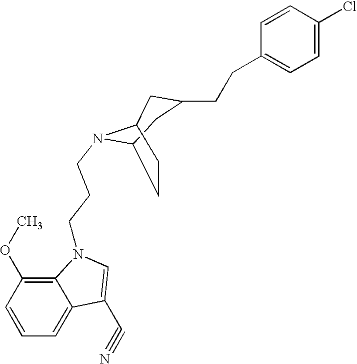 | 7.4 | 77 | 6.6 | |
| 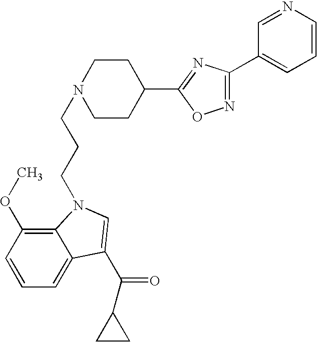 | 7.4 | 96 | | |
| 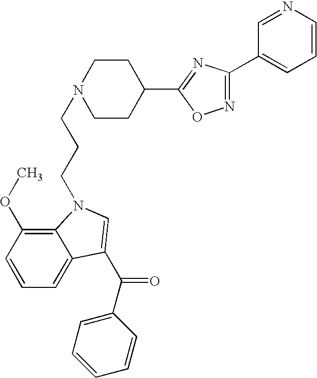 | 7.4 | 80 | | |
| 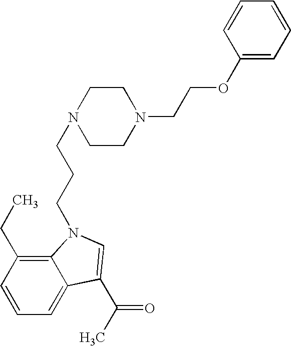 | 7.4 | 110 | | |
| 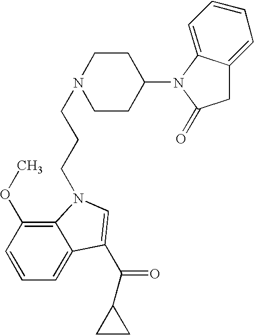 | 7.4 | 110 | | |
| 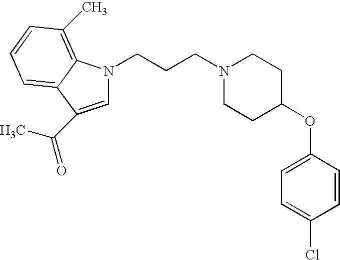 | 7.4 | 101 | 5.7 | |
| 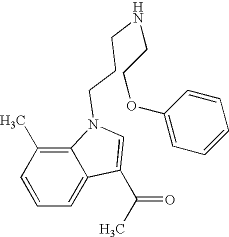 | 7.4 | 100 | | |
| 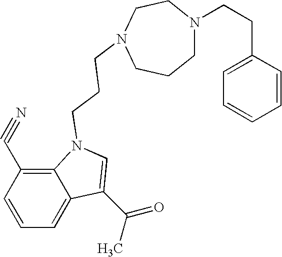 | 7.4 | 83 | | |
| 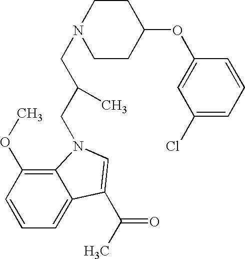 | 7.4 | 102 | 6.1 | |
| 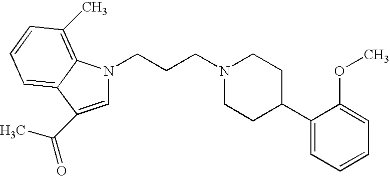 | 7.4 | 116 | 5.9 | |
| 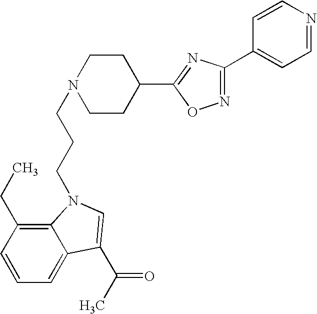 | 7.4 | 121 | | |
| 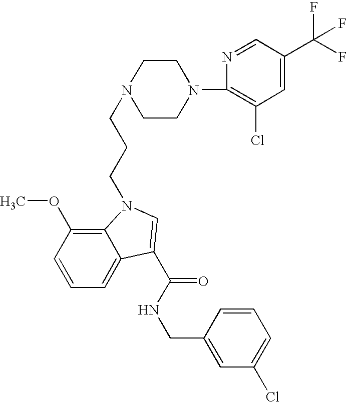 | 7.4 | 107 | | |
| 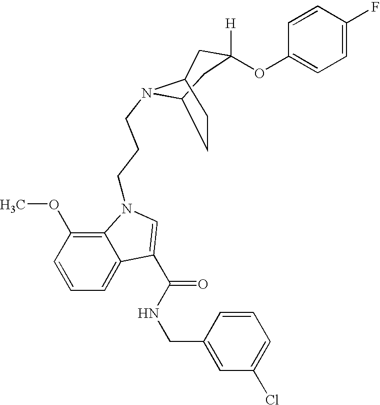 | 7.4 | 152 | 6.8 | |
| 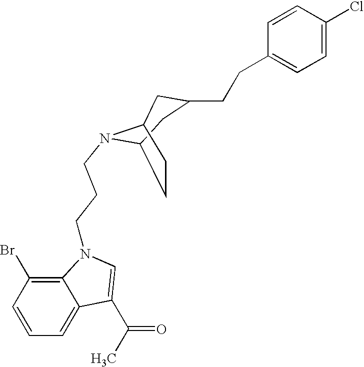 | 7.4 | 106 | | |
| 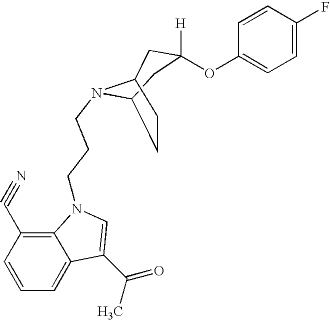 | 7.3 | 93 | 7.0 | |
| 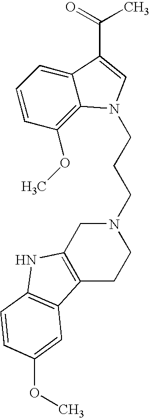 | 7.3 | 123 | 6.0 | |
| 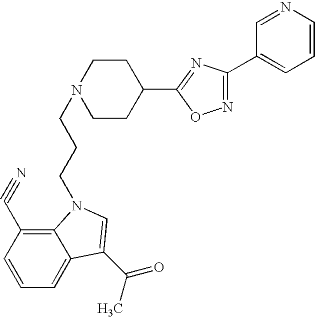 | 7.3 | 77 | | |
| 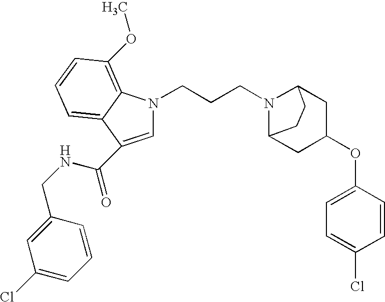 | 7.3 | 97 | 6.6 | |
| 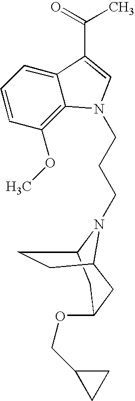 | 7.3 | 113 | 5.8 | |
| 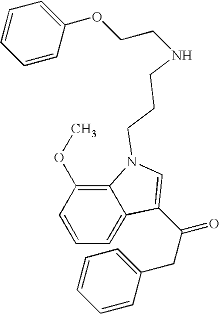 | 7.3 | 92 | | |
| 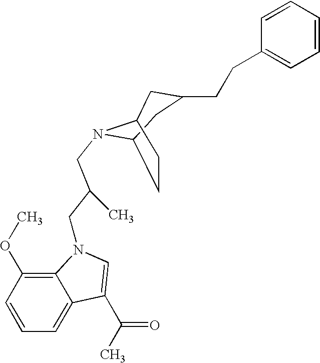 | 7.3 | 120 | | |
| 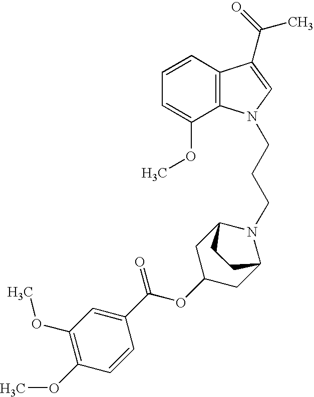 | 7.3 | 104 | 6.0 | |
| 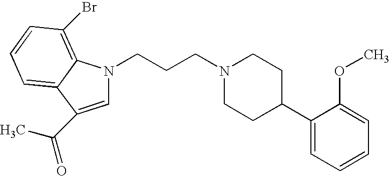 | 7.3 | 109 | 6.5 | |
| 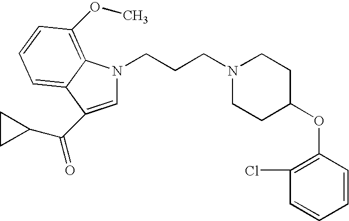 | 7.3 | 102 | 5.5 | |
| 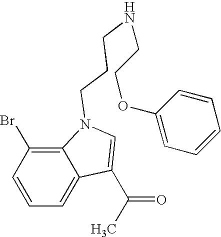 | 7.3 | 132 | | |
| 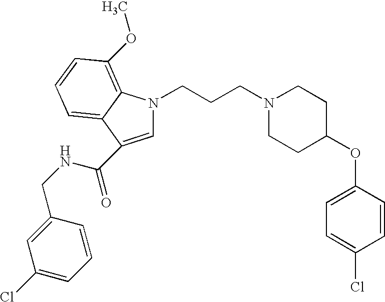 | 7.3 | 123 | 6.2 | |
| 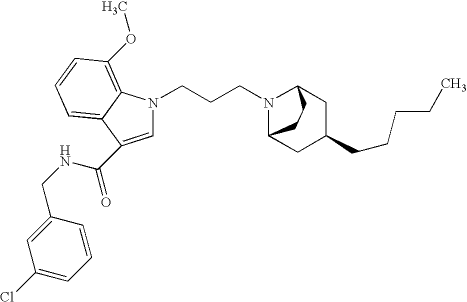 | 7.3 | 123 | 6.7 | |
| 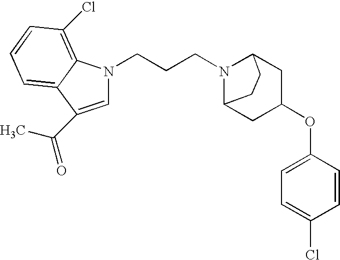 | 7.3 | 104 | 6.0 | |
| 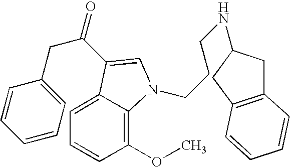 | 7.3 | 103 | | |
| 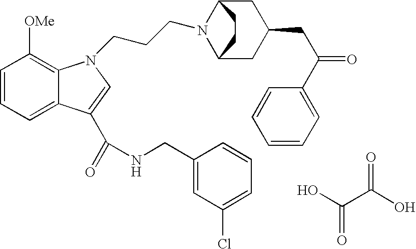 | 7.3 | 111 | 7.2 | |
| 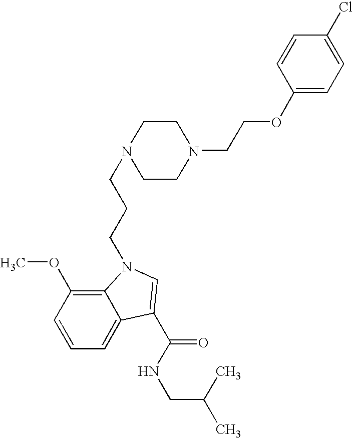 | 7.2 | 84 | — | |
| 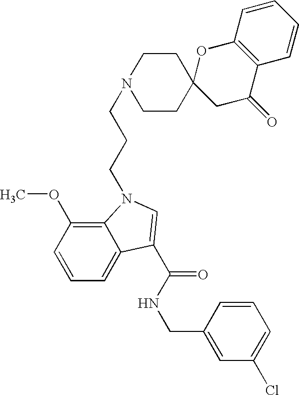 | 7.2 | 7.8 | | |
| 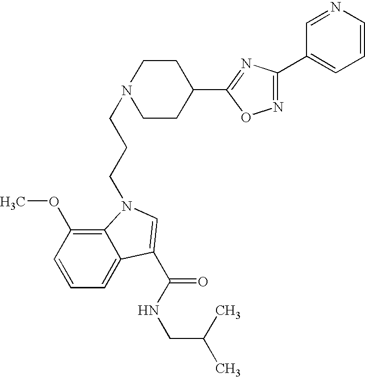 | 7.2 | 93 | | |
| 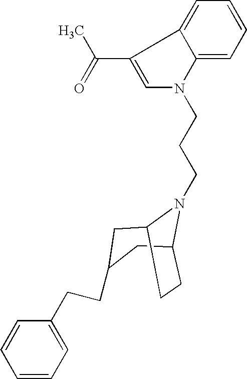 | 7.2 | 96 | 5.8 | |
| 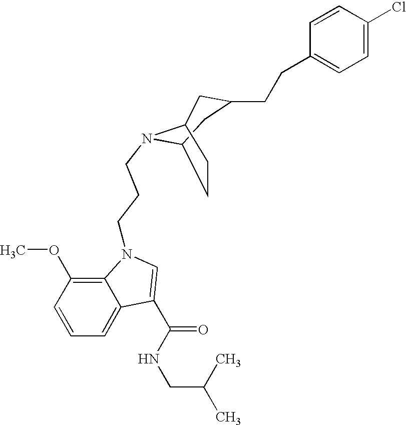 | 7.2 | 85 | | |
| 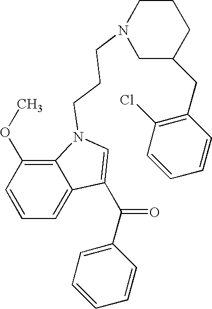 | 7.2 | 82 | | |
| 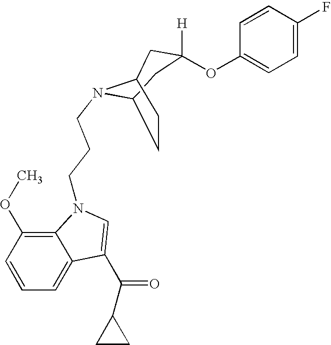 | 7.2 | 131 | | |
| 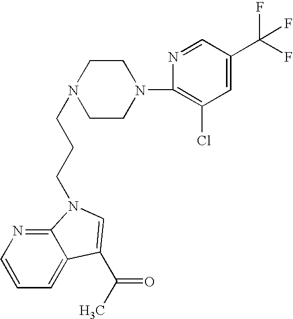 | 7.2 | 91 | | |
| 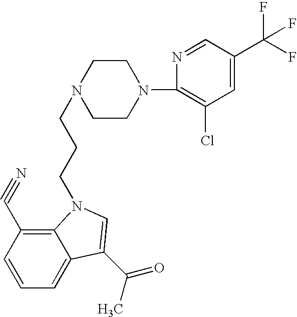 | 7.1 | 106 | | |
| 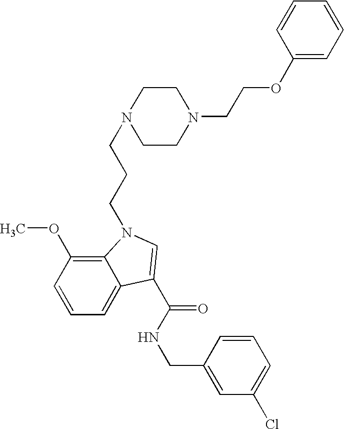 | 7.1 | 101 | 8.2 | 8.5 |
| 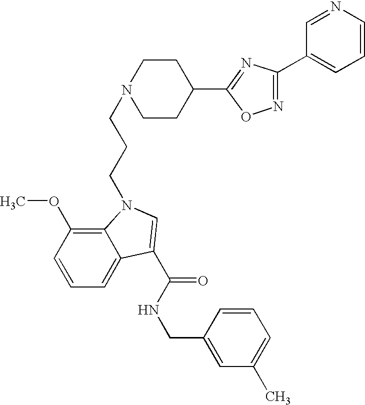 | 7.1 | 97 | 6.8 | |
| 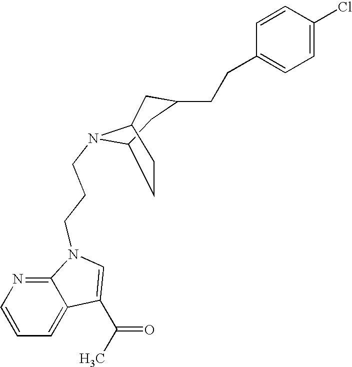 | 7.1 | 87 | 6.4 | |
| 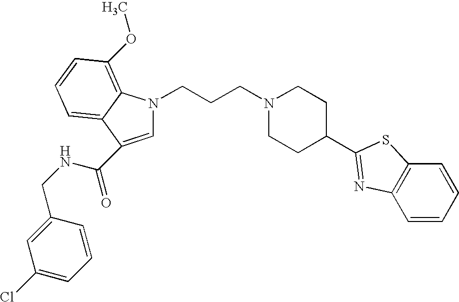 | 7.1 | 112 | 6.6 | |
| 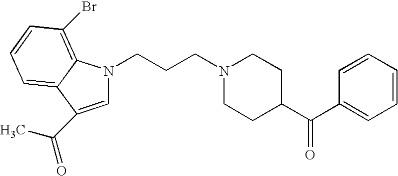 | 7.1 | 84 | 5.8 | |
| 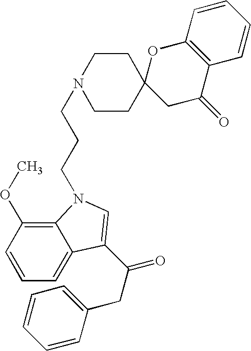 | 7.1 | 98 | | |
| 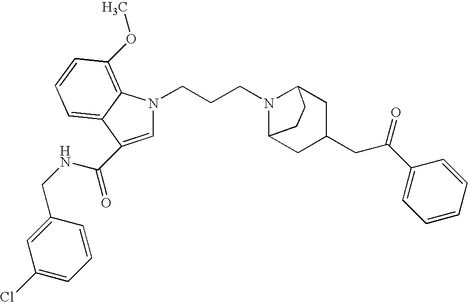 | 7.1 | 104 | 6.7 | |
| 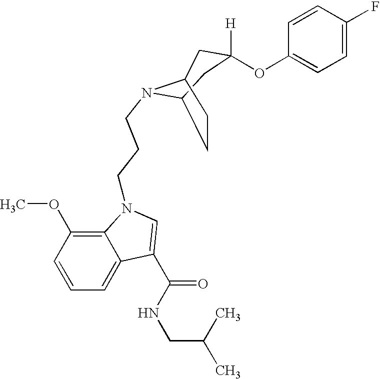 | 7.1 | 147 | | |
| 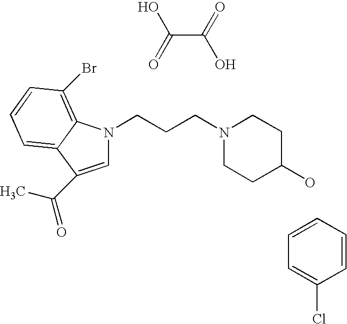 | 7.1 | 113 | | |
| 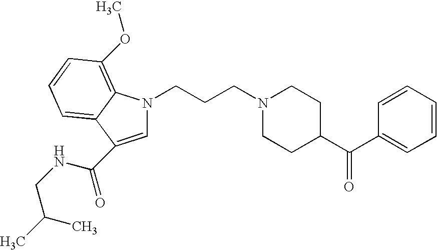 | 7.1 | 114 | | |
| 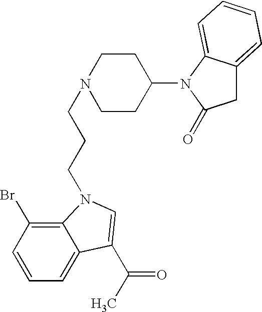 | 7.0 | 60 | | |
| 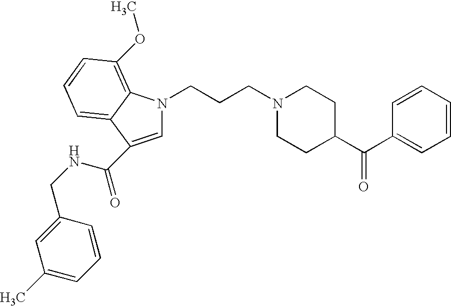 | 7.0 | 126 | | |
| 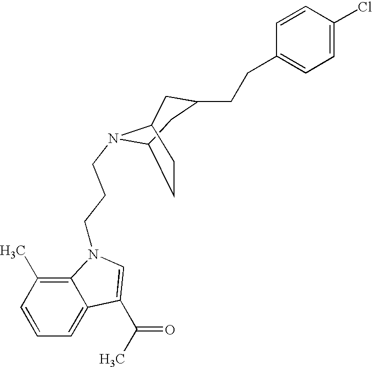 | 7.0 | 50 | | |
| 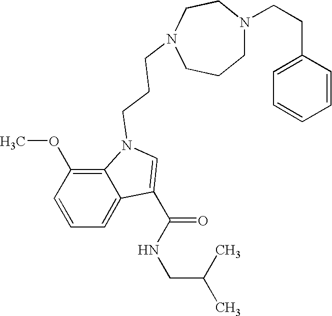 | 7.0 | 114 | 6.1 | |
| 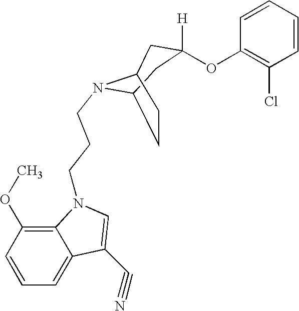 | 7.0 | 114 | 6.2 | |
| 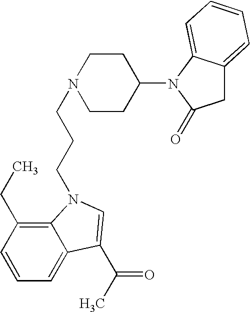 | 7.0 | 147 | | |
| 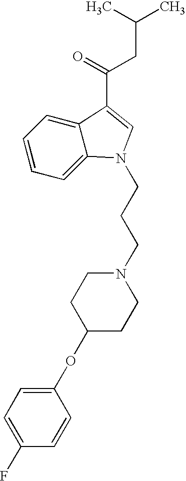 | 7.0 | 93 | 6.0 | |
| 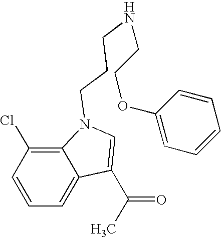 | 7.0 | 84 | | |
| 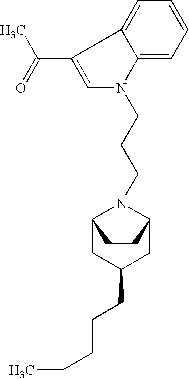 | 7.0 | 118 | 5.6 | |
| 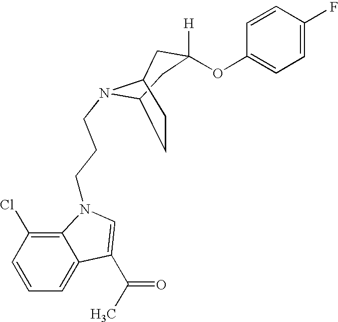 | 7.0 | 87 | — | |
| 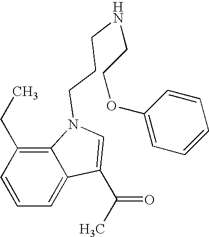 | 7.0 | 106 | 7.9 | |
| 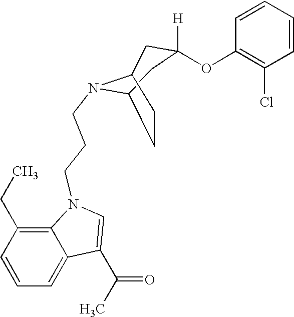 | 7.0 | 101 | — | |
| 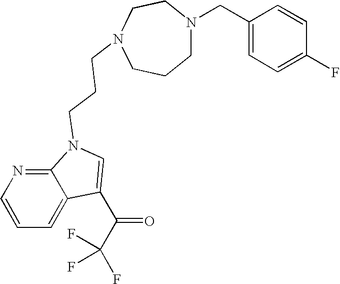 | 7.0 | 83 | | |
| 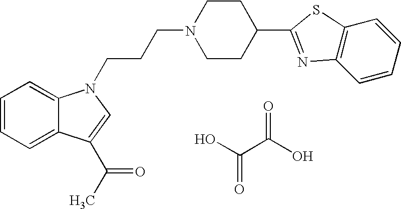 | 7.0 | 115 | 5.8 | |
| 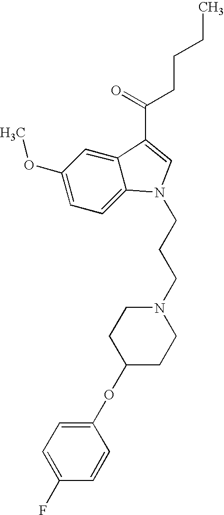 | 7.0 | 91 | | |
| 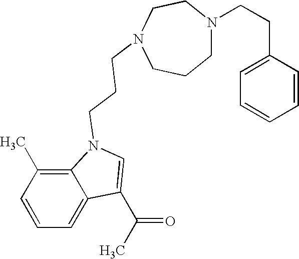 | 7.0 | 102 | | |
| 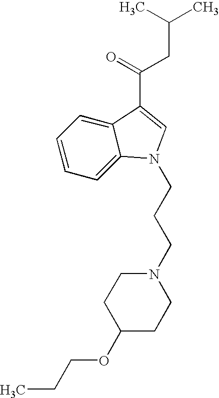 | 6.9 | 81 | | |
| 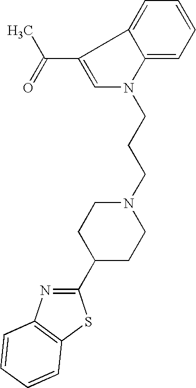 | 6.9 | 99 | 5.3 | |
| 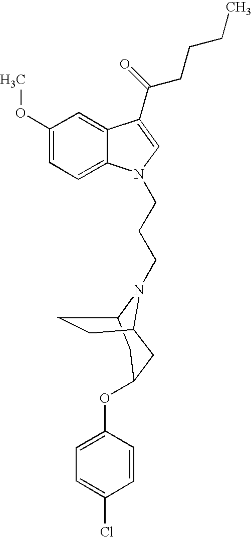 | 6.9 | 110 | | |
| 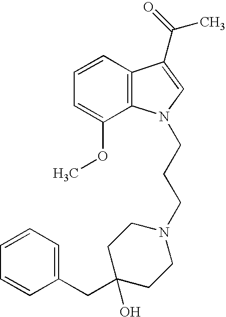 | 6.9 | 106 | | |
| 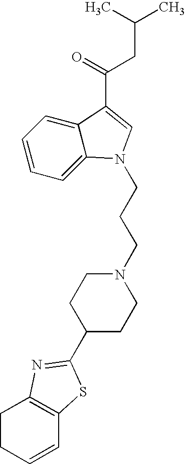 | 6.9 | 108 | | |
| 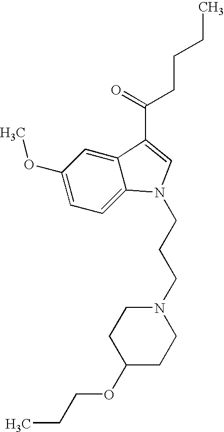 | 6.9 | 114 | | |
| 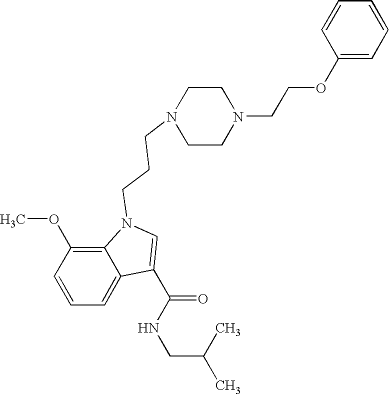 | 6.9 | 118 | | |
| 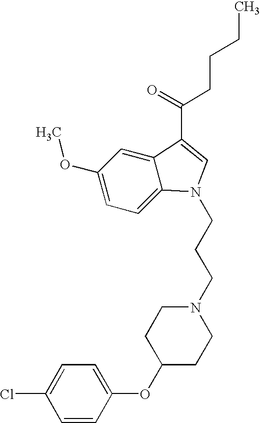 | 6.9 | 111 | 6.6 | |
| 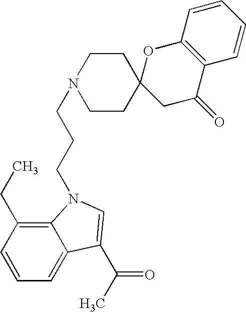 | 6.9 | 85 | | |
| 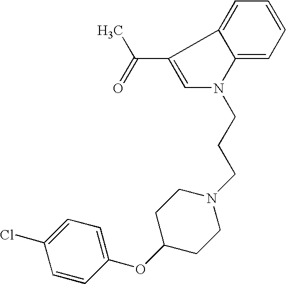 | 6.9 | 104 | 5.6 | |
| 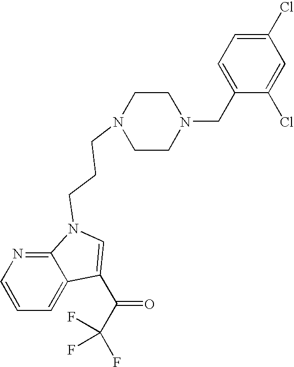 | 6.9 | 78 | | |
| 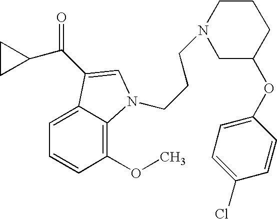 | 6.9 | 37 | | |
| 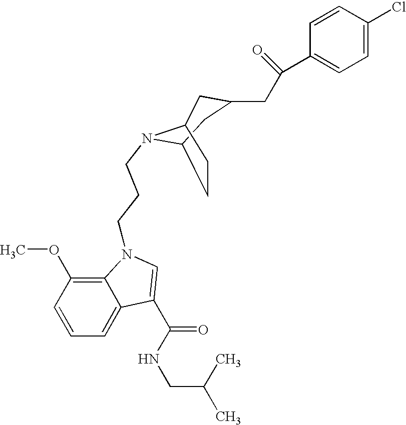 | 6.9 | 128 | | |
| 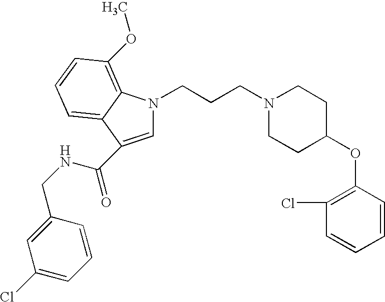 | 6.9 | 125 | | |
| 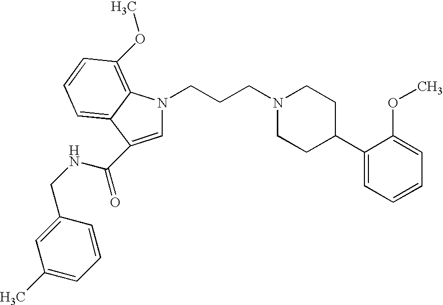 | 6.9 | 127 | | |
| 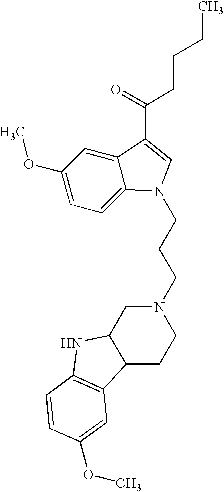 | 6.9 | 91 | | |
| 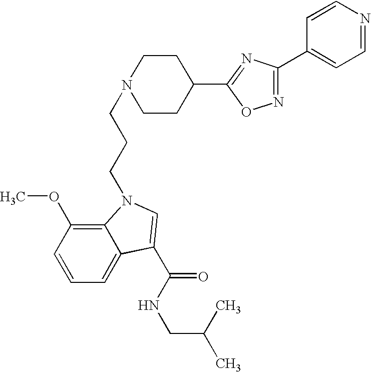 | 6.8 | 93 | | |
| 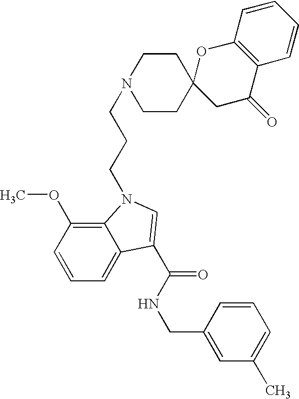 | 6.8 | 63 | | |
| 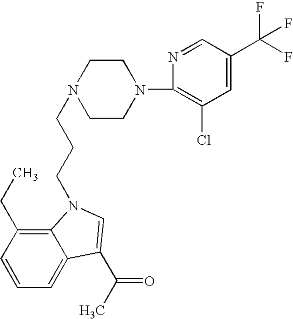 | 6.8 | 90 | | |
| 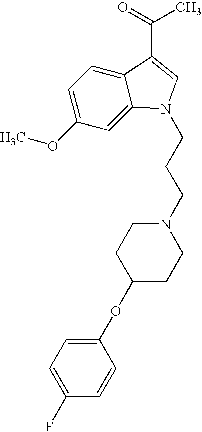 | 6.8 | 70 | | |
| 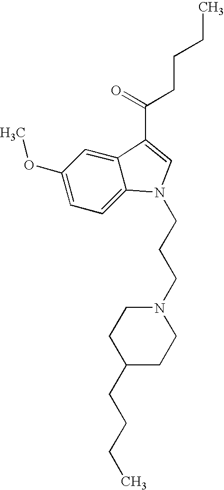 | 6.8 | 104 | | |
| 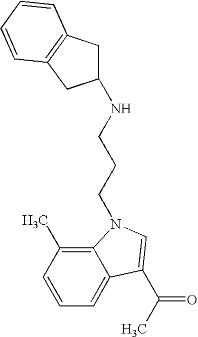 | 6.8 | 70 | | |
| 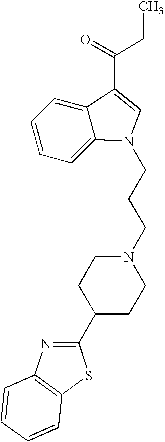 | 6.8 | 104 | | |
| 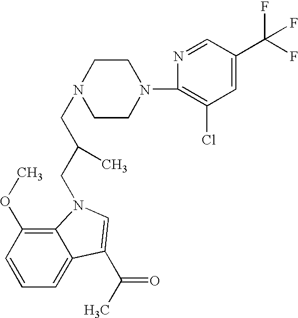 | 6.8 | 95 | | |
| 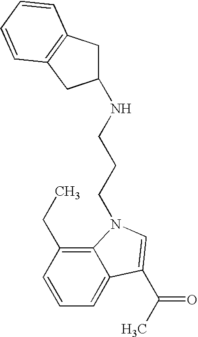 | 6.8 | 98 | | |
| 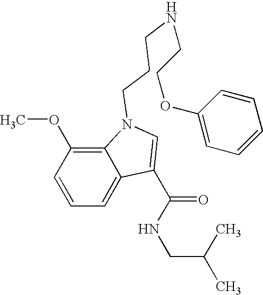 | 6.8 | 118 | | |
| 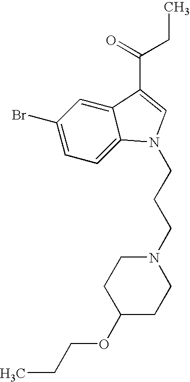 | 6.8 | 90 | 5.0 | |
| 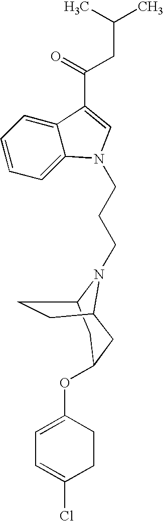 | 6.8 | 137 | | |
| 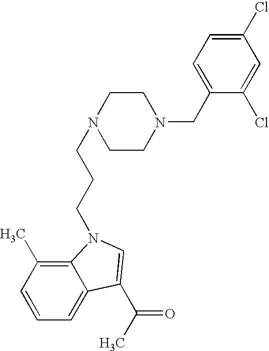 | 6.8 | 120 | | |
| 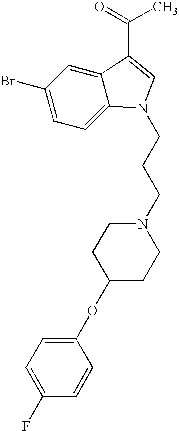 | 6.8 | 75 | | |
| 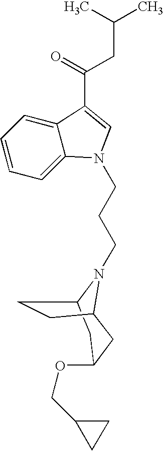 | 6.8 | 129 | | |
| 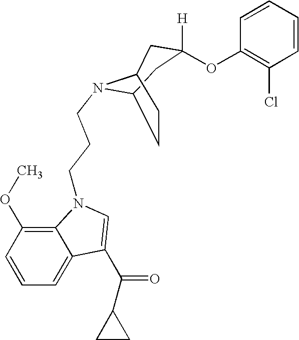 | 6.8 | 130 | | |
| 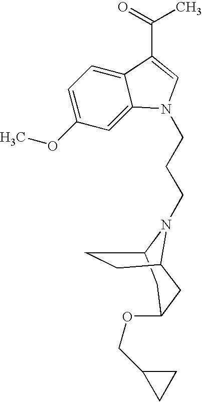 | 6.8 | 93 | | |
| 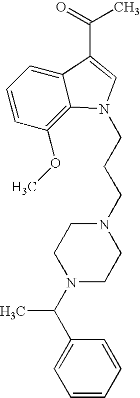 | 6.8 | 114 | | |
| 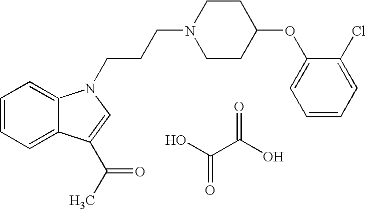 | 6.8 | 105 | | |
| 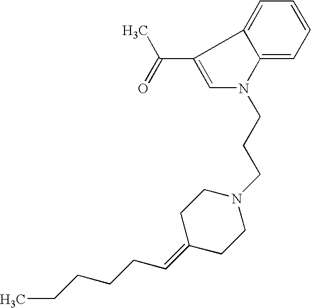 | 6.7 | 139 | 6.7 | |
| 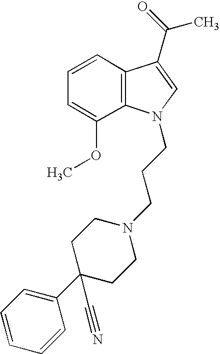 | 67 | 101 | | |
| 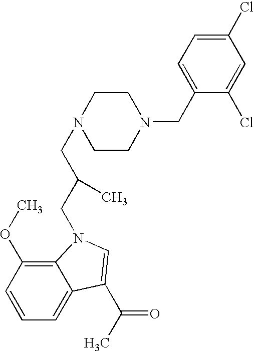 | 6.7 | 78 | | |
| 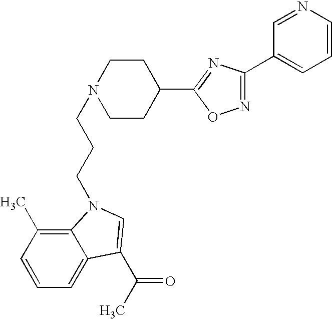 | 6.7 | 103 | | |
| 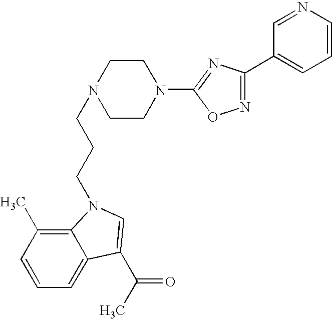 | 6.7 | 110 | | |
| 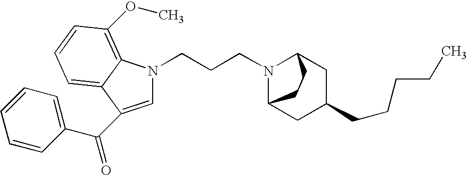 | 6.7 | 91 | | |
| 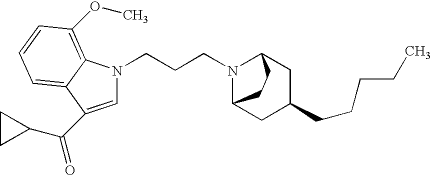 | 6.7 | 101 | | |
| 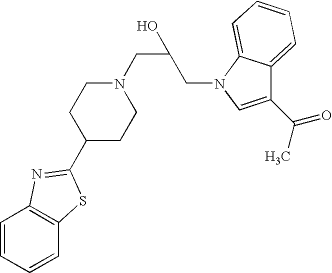 | 6.7 | 58 | | |
| 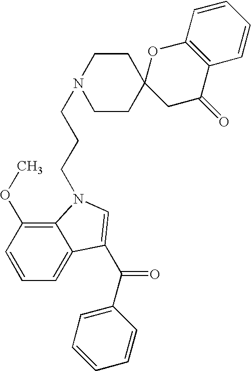 | 6.7 | 60 | | |
| 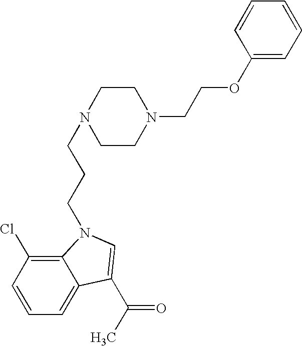 | 6.7 | 91 | | |
| 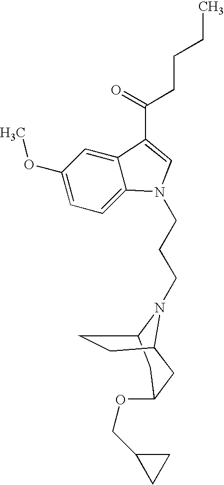 | 6.7 | 132 | | |
| 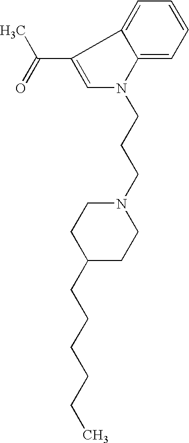 | 6.7 | 94 | 5.2 | |
| 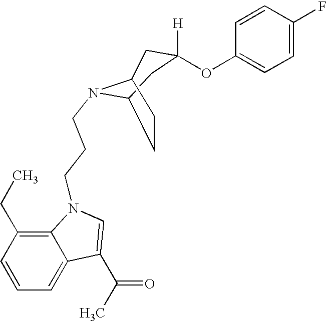 | 6.7 | 98 | — | |
| 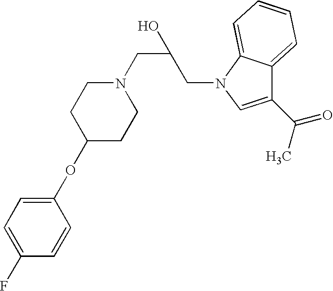 | 6.7 | 74 | | |
| 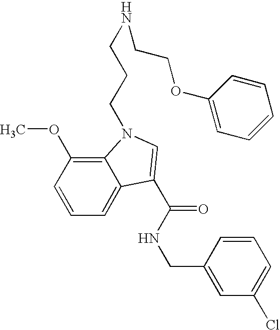 | 6.6 | 120 | 7.4 | |
| 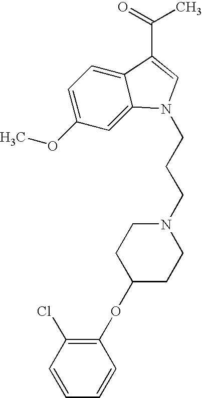 | 6.6 | 88 | | |
| 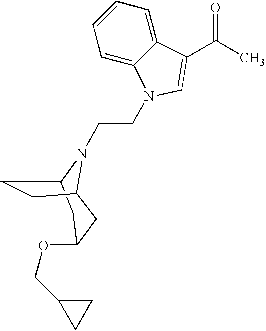 | 6.6 | 54 | | |
| 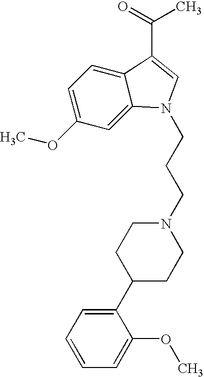 | 6.6 | 72 | | |
| 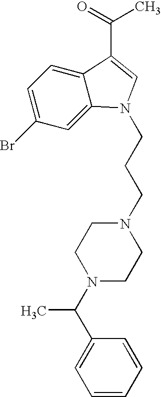 | 6.6 | 93 | | |
| 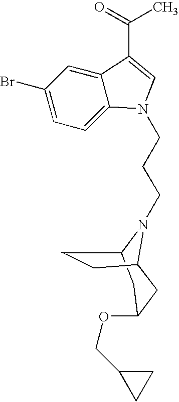 | 6.6 | 102 | | |
| 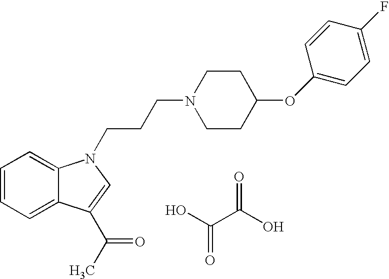 | 6.6 | 128 | | |
| 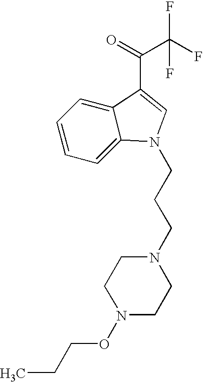 | 6.6 | 99 | | |
| 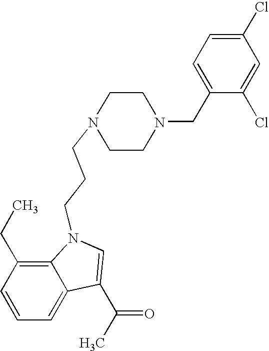 | 6.6 | 106 | | |
| 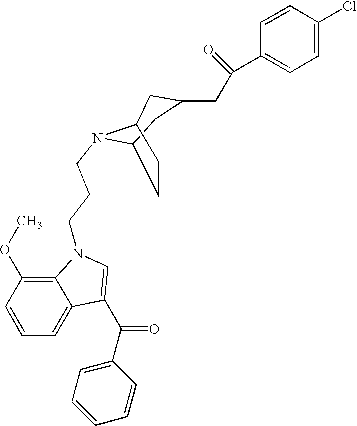 | 6.6 143 | | | |
| 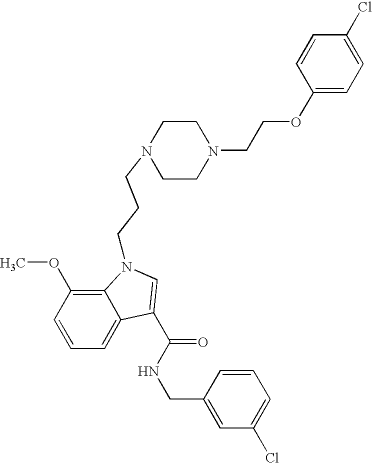 | 6.6 | 104 | 7.8 | |
| 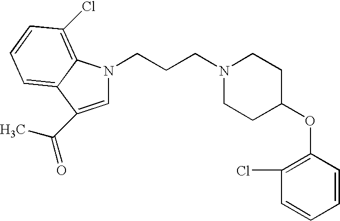 | 6.6 | 97 | | |
| 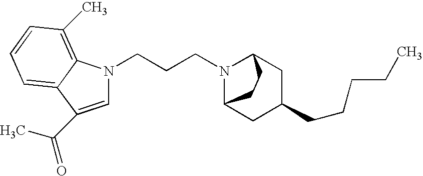 | 6.6 | 105 | | |
| 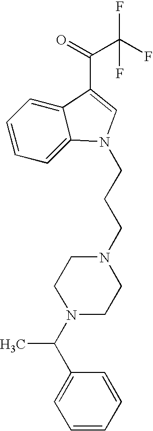 | 6.6 | 92 | | |
| 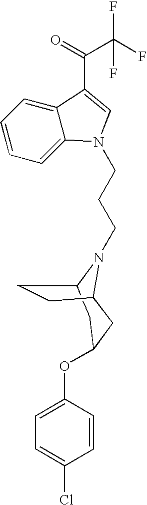 | 6.6 | 97 | | |
| 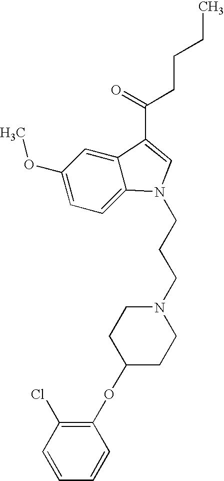 | 6.6 | 92 | | |
| 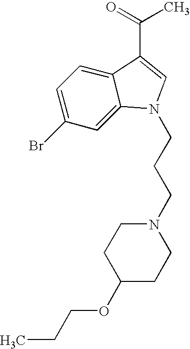 | 6.6 | 107 | | |
| 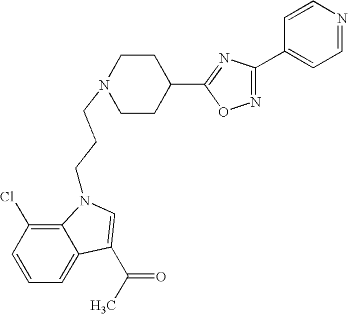 | 6.6 | 108 | — | |
| 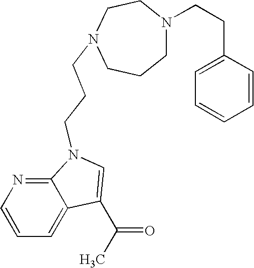 | 6.6 | 84 | | |
| 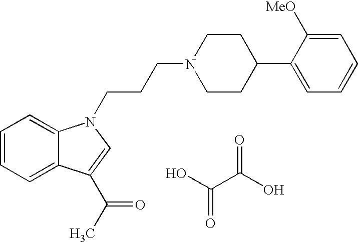 | 6.5 | 109 | | |
| 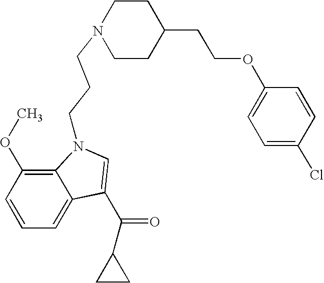 | 6.5 | 89 | | |
| 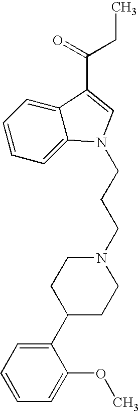 | 6.5 | 59 | | |
| 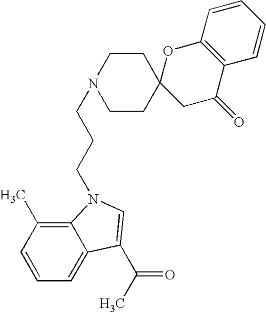 | 6.5 | 71 | | |
| 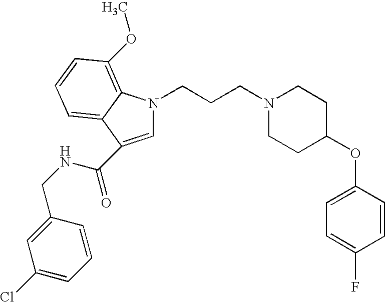 | 6.5 | 106 | | |
| 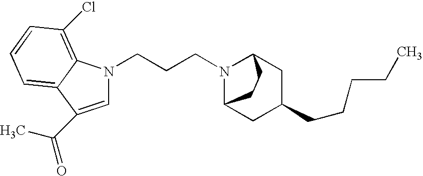 | 6.5 | 72 | | |
| 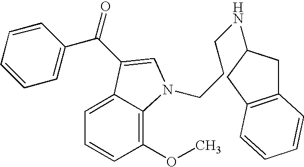 | 6.5 | 93 | | |
| 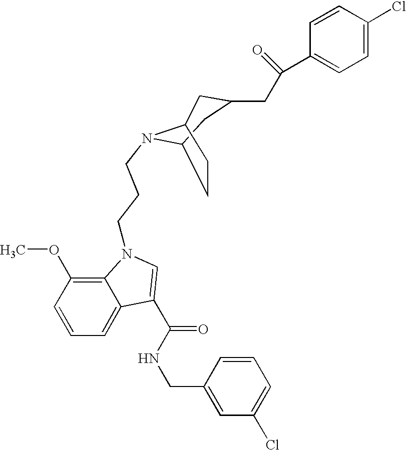 | 6.5 | 76 | | |
| 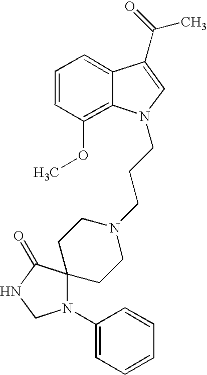 | 6.5 | 38 | | |
| 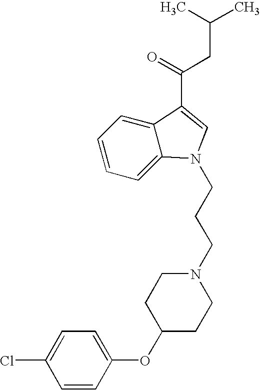 | 6.5 | 112 | | |
| 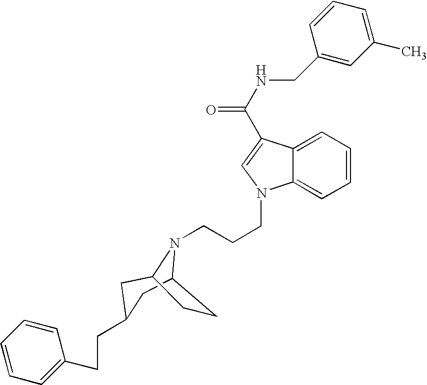 | 6.5 | 108 | 6.2 | |
| 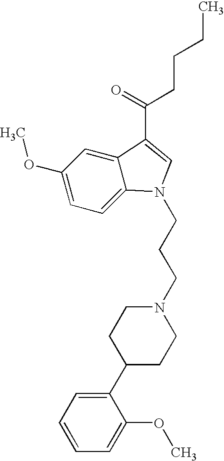 | 6.5 | 101 | | |
| 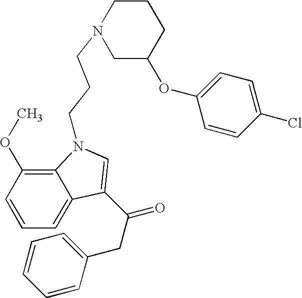 | 6.5 | 86 | | |
| 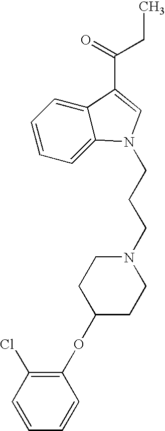 | 6.5 | 76 | | |
| 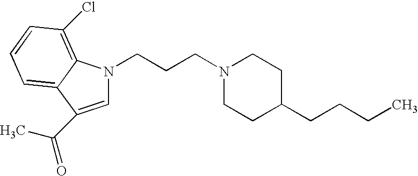 | 6.5 | 80 | | |
| 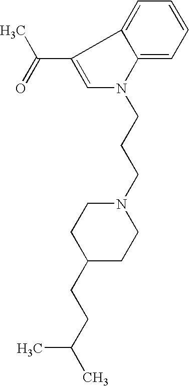 | 6.4 | 73 | 5.4 | |
| 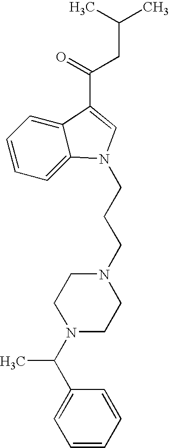 | 6.4 | 106 | | |
| 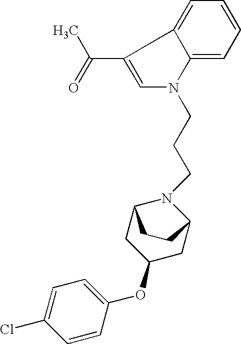 | 6.4 | 107 | 5.1 | |
| 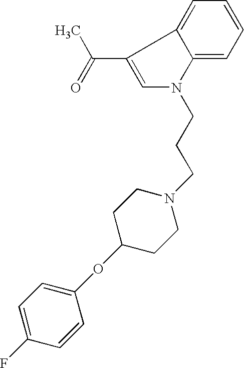 | 6.4 | 96 | 5.7 | |
| 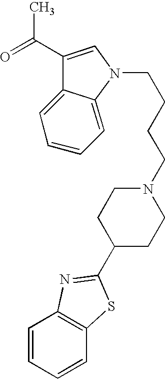 | 6.4 | 81 | | |
| 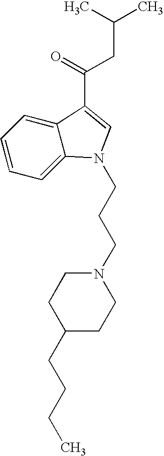 | 6.4 | 85 | | |
| 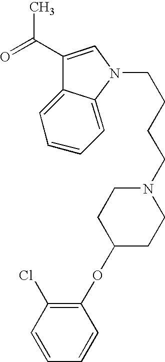 | 6.4 | 46 | | |
| 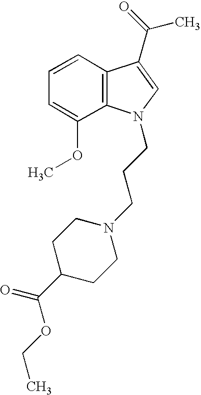 | 6.4 | 127 | | |
| 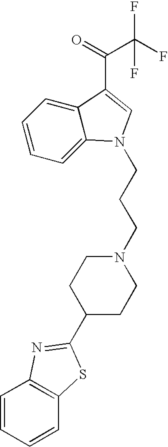 | 6.4 | 88 | | |
| 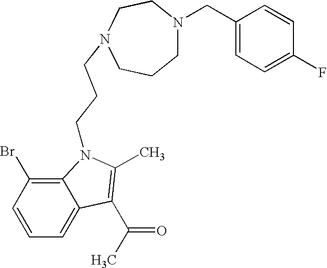 | 6.4 | 49 | | |
| 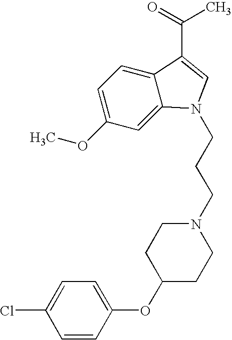 | 6.4 | 67 | | |
| 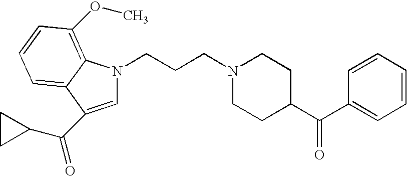 | 6.4 | 100 | | |
| 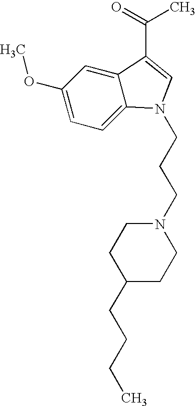 | 6.4 | 118 | | |
| 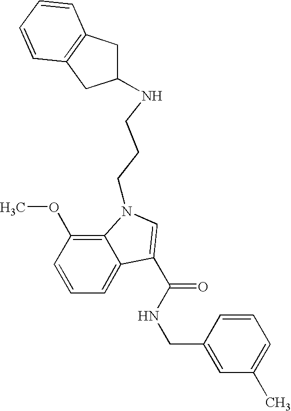 | 6.4 | 84 | 7.1 | |
| 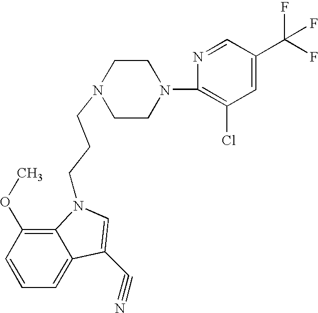 | 6.4 | 97 | | |
| 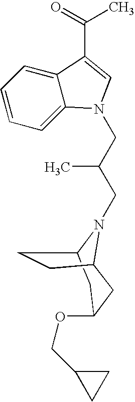 | 6.4 | 65 | | |
| 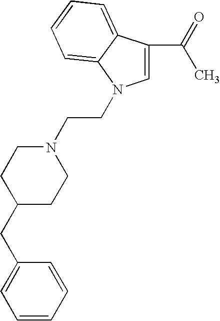 | 6.4 | 77 | | |
| 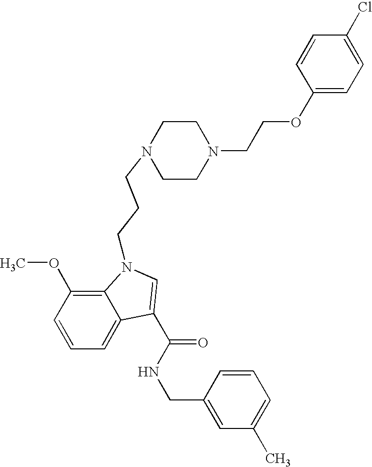 | 6.3 | 79 | 6.9 | |
| 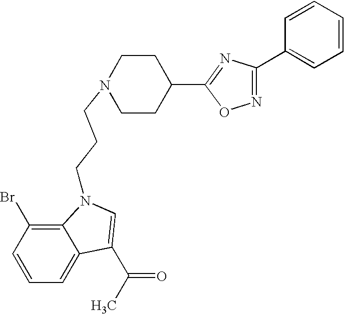 | 6.3 | 69 | | |
| 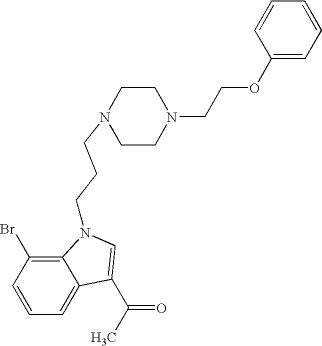 | 6.3 | 91 | | |
| 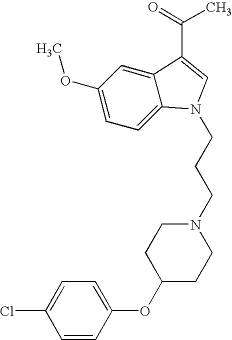 | 6.3 104 | | | |
| 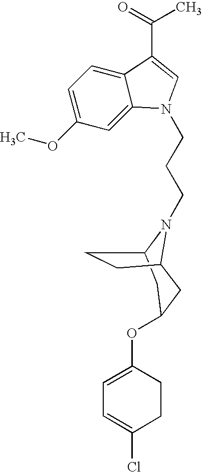 | 6.3 | 108 | | |
| 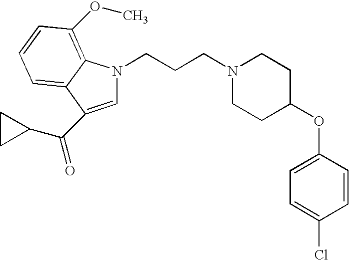 | 6.3 | 90 | | |
| 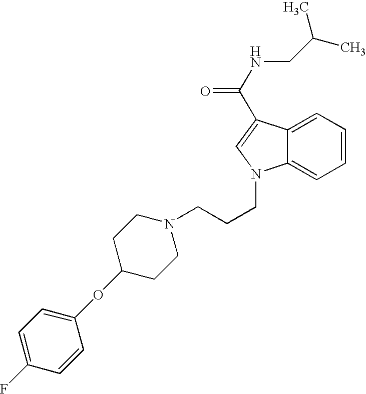 | 6.3 | 94 | | |
| 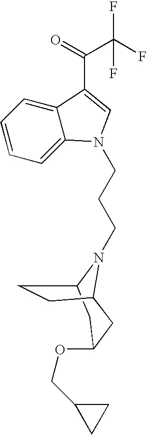 | 6.3 | 103 | | |
| 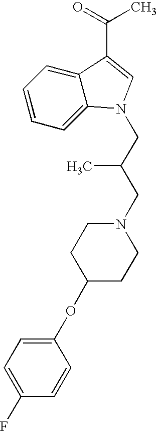 | 6.3 | 98 | | |
| 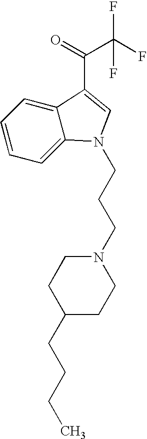 | 6.3 | 74 | | |
| 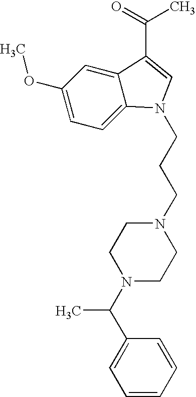 | 6.3 | 77 | | |
| 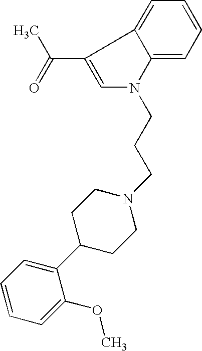 | 6.3 | 106 | 5.9 | |
| 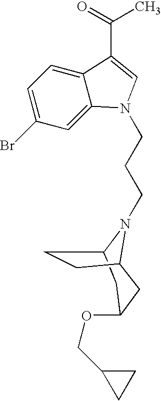 | 6.3 | 130 | | |
| 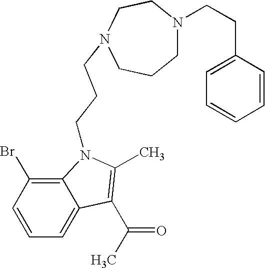 | 6.3 | 42 | | |
| 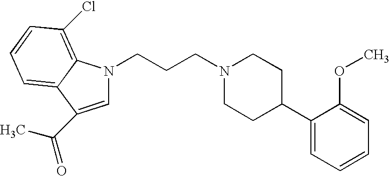 | 6.3 | 103 | | |
| 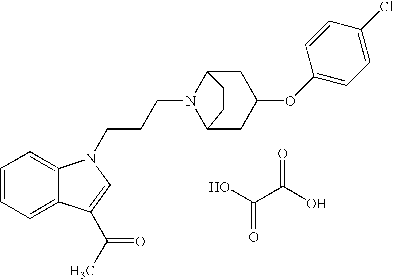 | 6.3 | 108 | | |
| 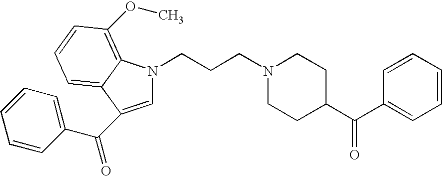 | 6.3 | 102 | | |
| 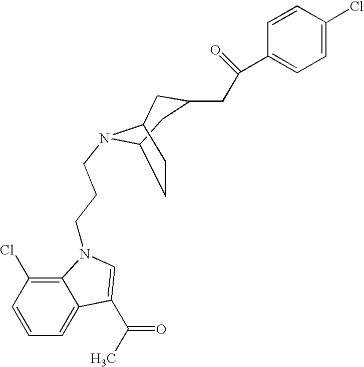 | 6.3 | 41 | | |
| 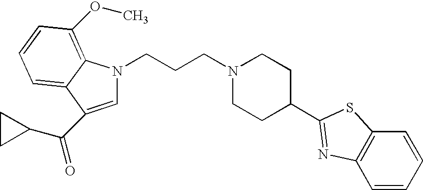 | 6.3 | 88 | | |
| 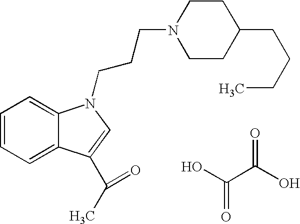 | 6.2 | 77 | 5.4 | |
| 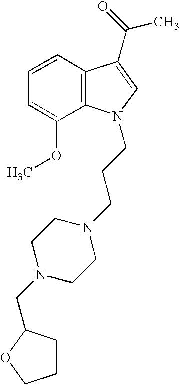 | 6.2 | 98 | | |
| 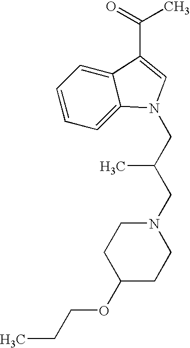 | 6.2 | 74 | | |
| 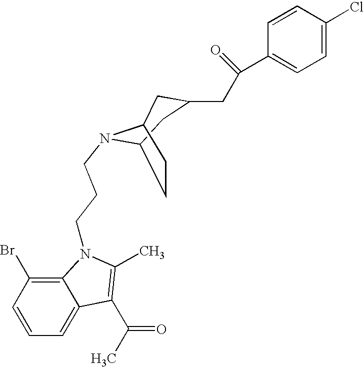 | 6.2 | 64 | 7.2 | |
| 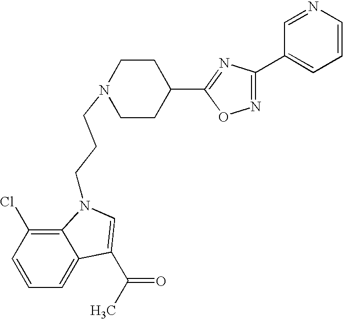 | 6.2 | 101 | | |
| 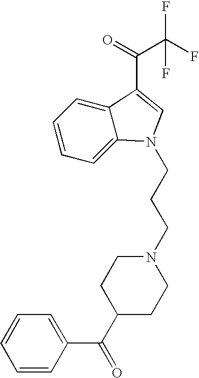 | 6.2 | 63 | | |
| 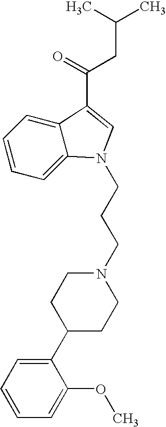 | 6.2 | 100 | | |
| 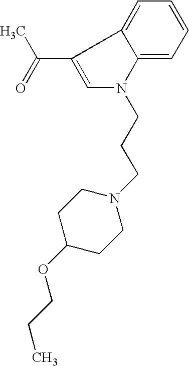 | 6.2 | 112 | | |
| 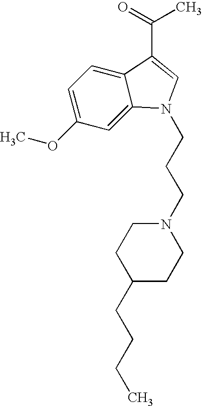 | 6.2 | 63 | | |
| 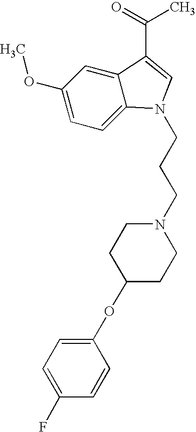 | 6.2 | 58 | | |
| 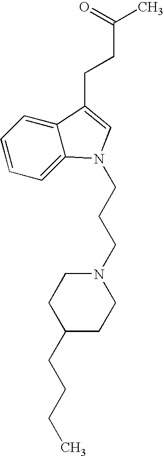 | 6.2 | 76 | | |
| 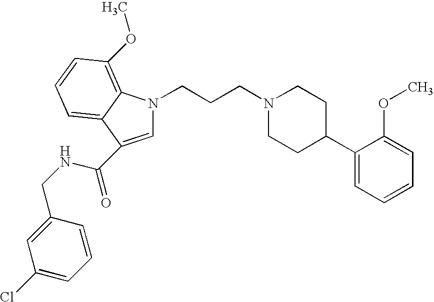 | 6.2 | 127 | | |
| 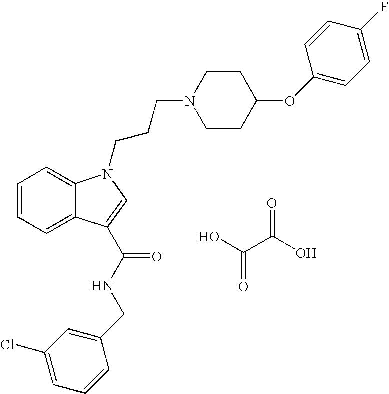 | 6.2 | 142 | | |
| 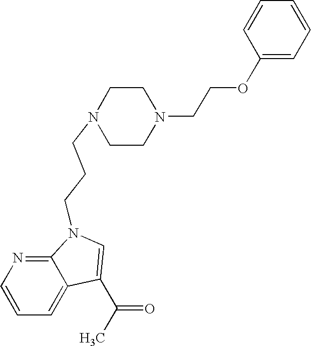 | 6.2 | 81 | | |
| 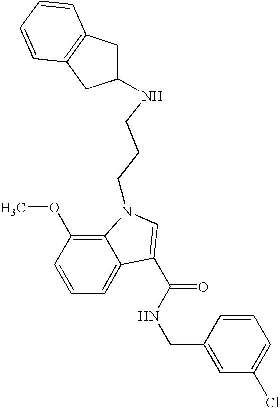 | 6.1 | 133 | 7.2 | |
| 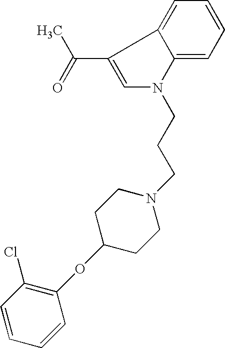 | 6.1 | 116 | 5.6 | |
| 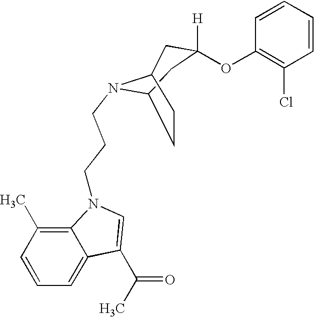 | 6.1 | 101 | | |
| 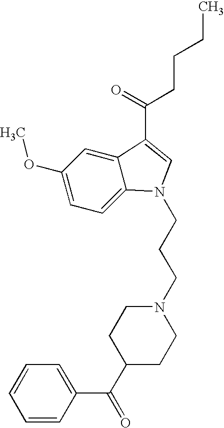 | 6.1 | 98 | | |
| 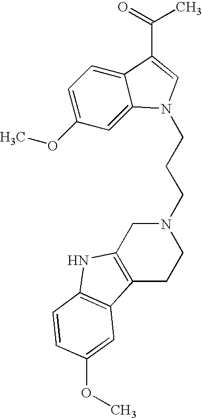 | 6.1 | 69 | | |
| 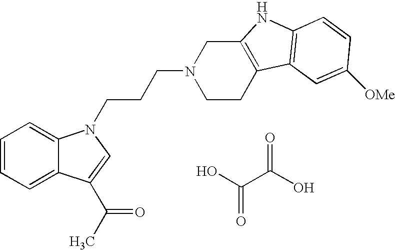 | 6.1 76 | | | |
| 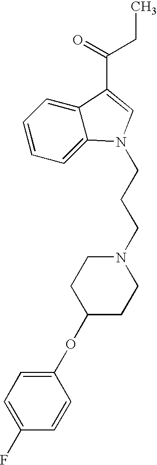 | 6.1 | 90 | | |
| 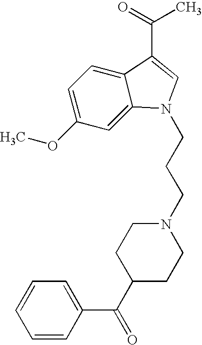 | 6.1 | 66 | | |
| 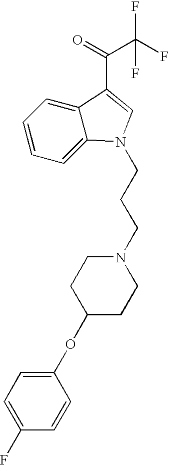 | 6.1 | 75 | | |
| 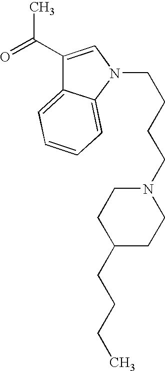 | 6.1 | 113 | | |
| 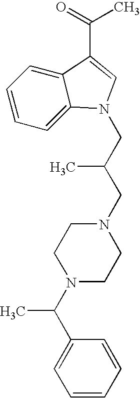 | 6.0 | 35 | | |
| 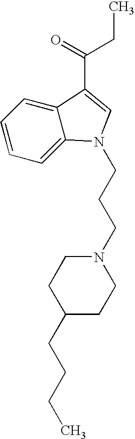 | 6.0 | 92 | | |
| 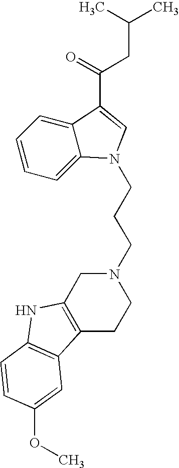 | 6.0 | 123 | | |
| 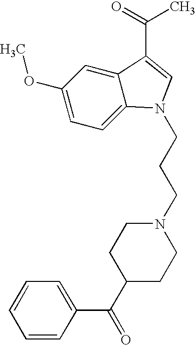 | 6.0 | 72 | | |
| 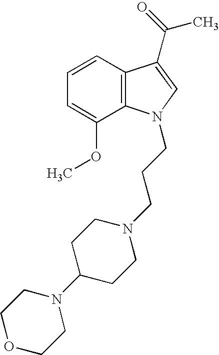 | 6.0 | 95 | | |
| 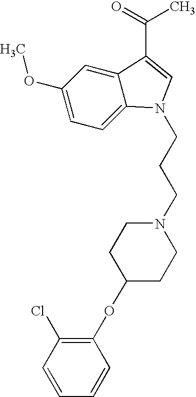 | 6.0 | 95 | | |
| 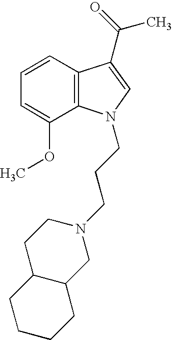 | 6.0 | 69 | | |
| 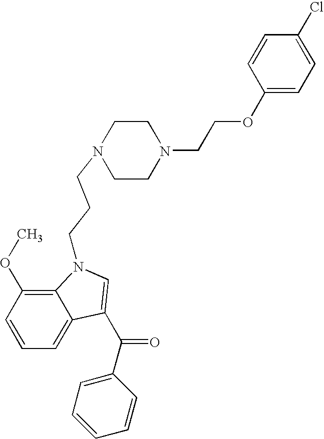 | 6.0 | 66 | 6.9 | |
| 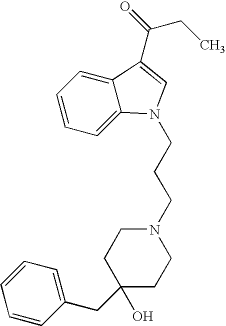 | 6.0 | 73 | | |
| 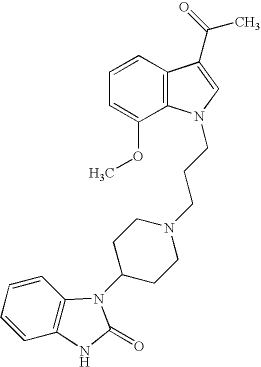 | 6.0 | 90 | | |
| 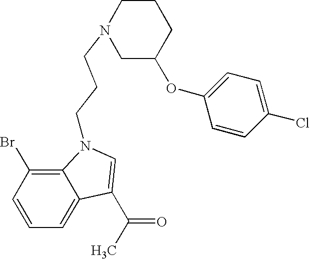 | 5.9 | 46 | | |
| 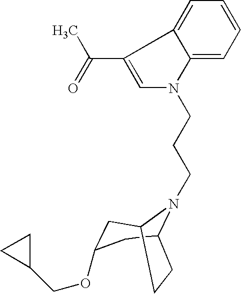 | 5.9 | 89 | 4.8 | |
| 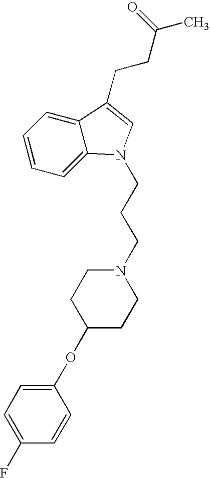 | 5.9 | 80 | | |
| 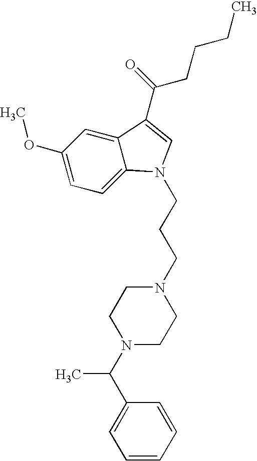 | 5.9 | 81 | | |
| 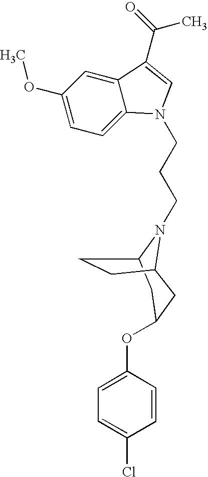 | 5.9 | 105 | | |
| 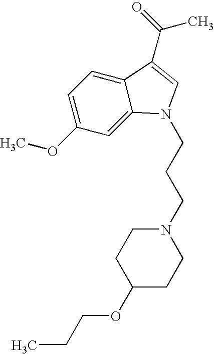 | 5.9 | 61 | | |
| 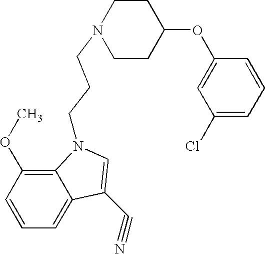 | 5.9 | 162 | 7.4 | |
| 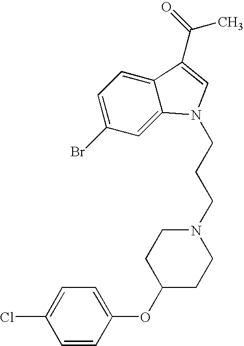 | 5.9 | 74 | | |
| 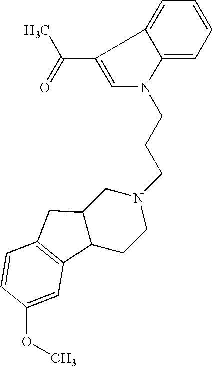 | 5.9 | 110 | 5.3 | |
| 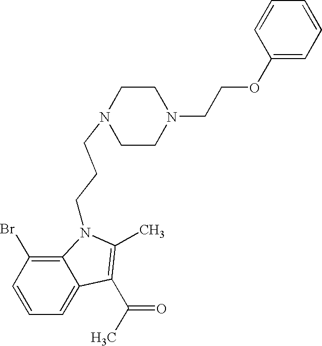 | 5.9 | 57 | | |
| 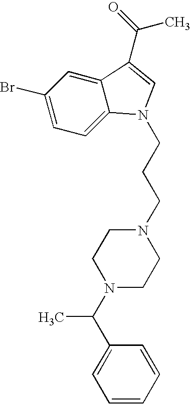 | 5.9 | 72 | | |
| 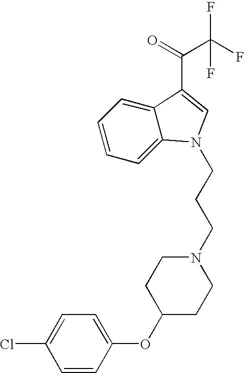 | 5.8 | 77 | | |
| 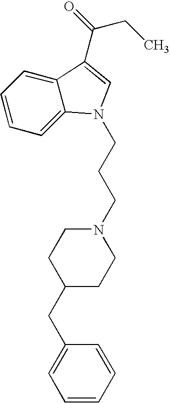 | 5.8 | 109 | | |
| 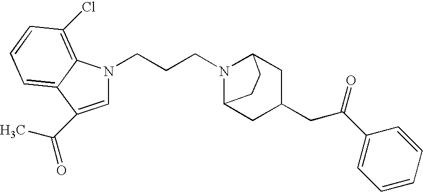 | 5.8 | 85 | | |
| 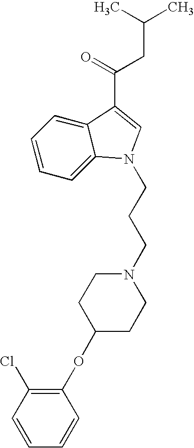 | 5.8 | 96 | | |
| 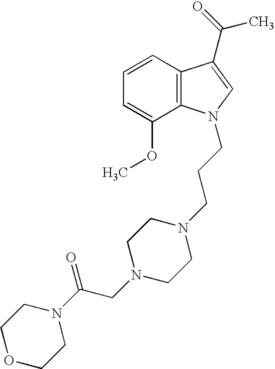 | 5.8 | 37 | | |
| 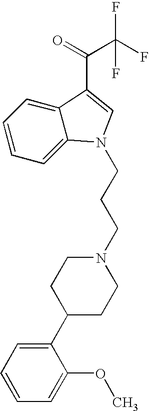 | 5.8 | 82 | | |
| 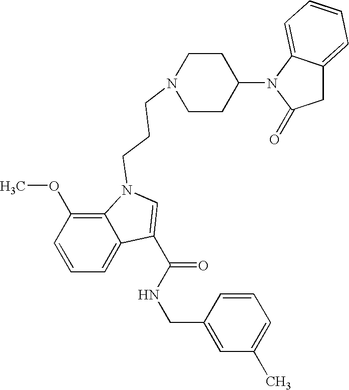 | 5.7 | 43 | | |
| 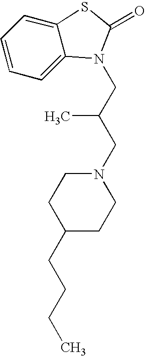 | 5.7 | 84 | | |
| 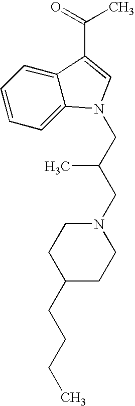 | 5.7 | 76 | | |
| 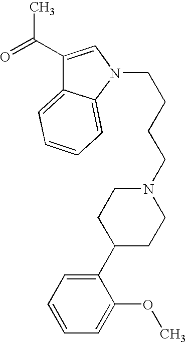 | 5.7 | 58 | | |
| 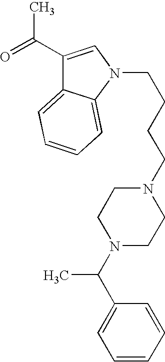 | 5.7 | 69 | | |
| 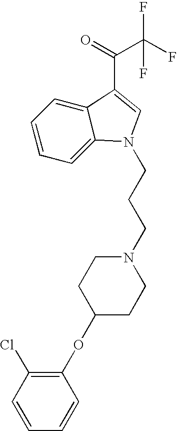 | 5.7 | 81 | | |
| 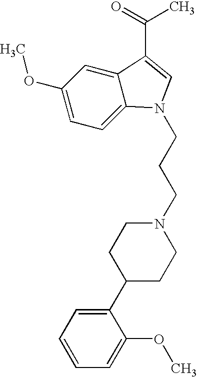 | 5.7 | 66 | | |
| 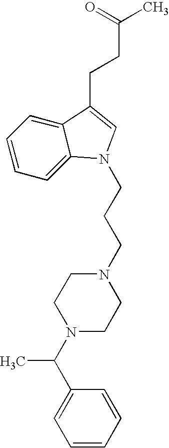 | 5.7 | 46 | | |
| 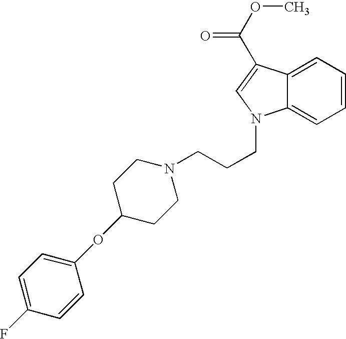 | 5.7 | 87 | 5.5 | |
| 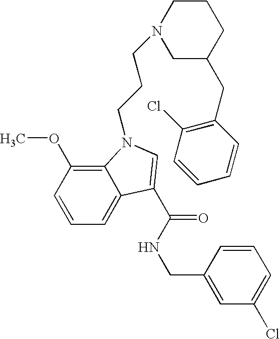 | 5.7 | 47 | | |
| 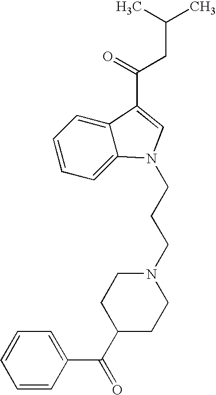 | 5.7 | 67 | | |
| 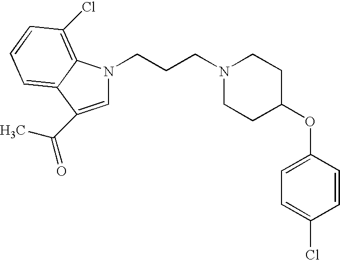 | 5.7 | 107 | | |
| 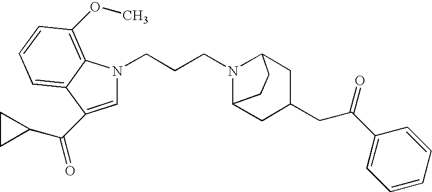 | 5.7 | 86 | | |
| 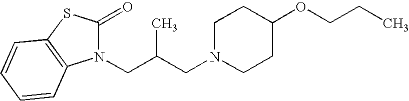 | 5.6 | 71 | | |
| 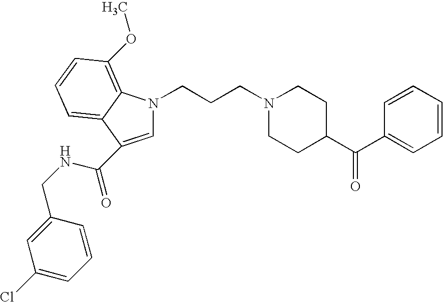 | 5.6 | 114 | | |
| 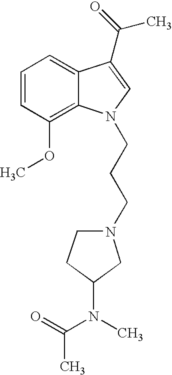 | 5.6 | 60 | | |
| 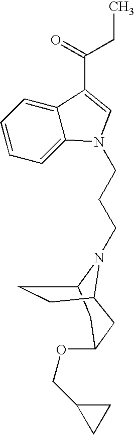 | 5.6 | 120 | | |
| 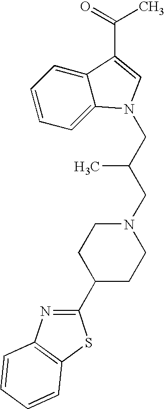 | 5.6 | 78 | | |
| 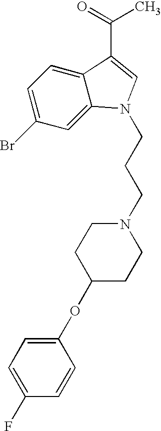 | 5.6 | 64 | | |
| 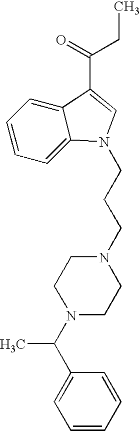 | 5.6 | 57 | | |
| 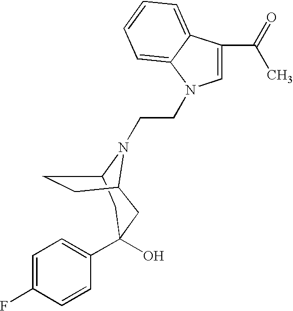 | 5.6 | −11 | | |
| 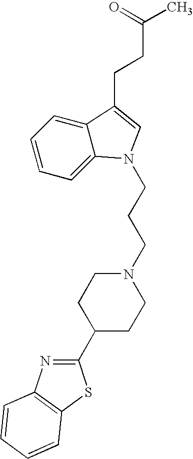 | 5.6 | 84 | | |
| 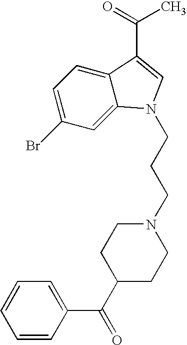 | 5.6 | 36 | | |
| 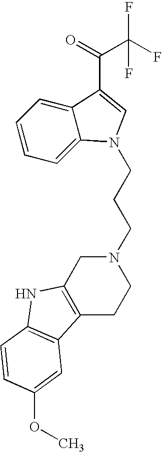 | 5.6 | 106 | | |
| 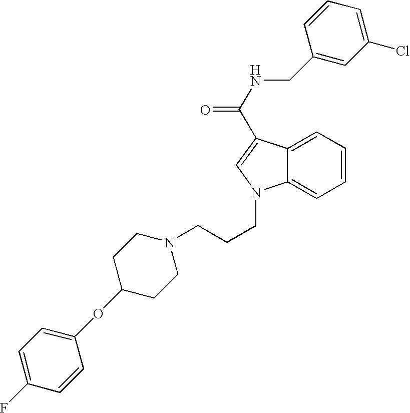 | 5.5 | 67 | 5.8 | |
| 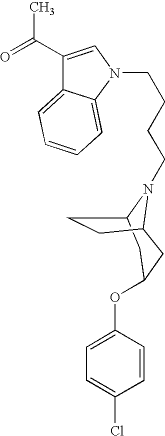 | 5.5 | 119 | | |
| 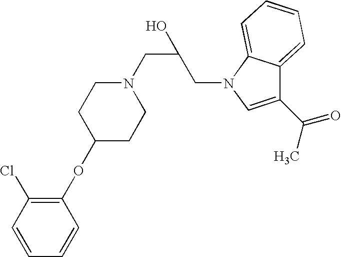 | 5.5 | 69 | | |
| 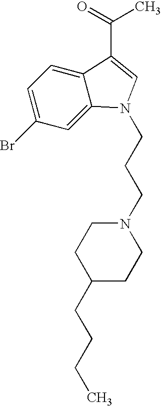 | 5.5 | 93 | | |
| 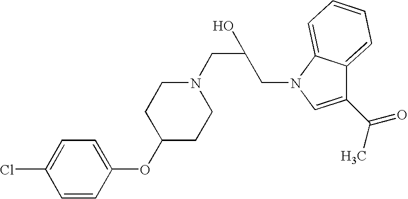 | 5.5 | 73 | | |
| 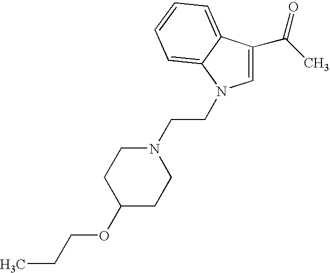 | 5.5 | 40 | | |
| 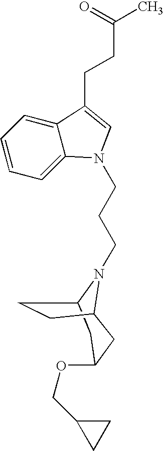 | 5.5 | 103 | | |
| 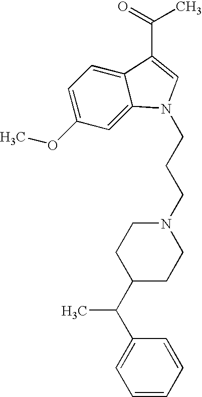 | 5.5 | 73 | | |
| 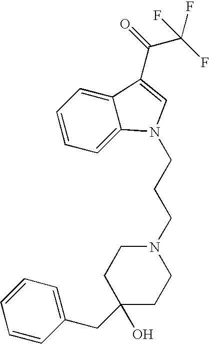 | 5.4 | 111 | | |
| 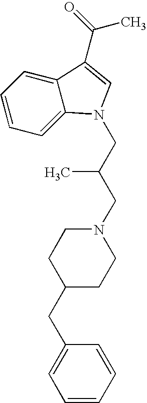 | 5.4 | 121 | | |
| 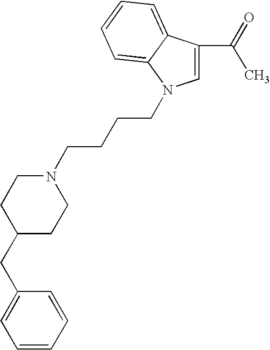 | 5.4 | 86 | | |
| 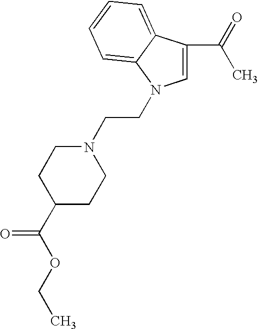 | 5.4 | 72 | | |
| 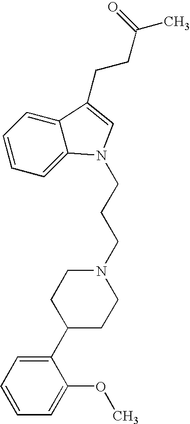 | 5.3 | 78 | | |
| 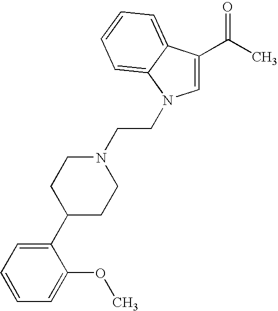 | 5.3 | 44 | | |
| 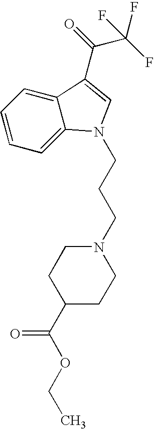 | 5.3 | 64 | | |
| 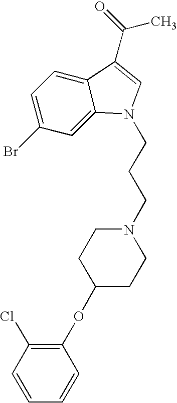 | 5.3 | 67 | | |
| 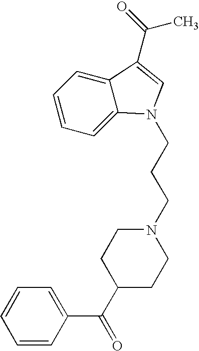 | 5.3 | 64 | | |
| 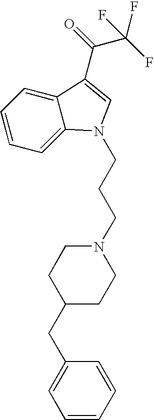 | 5.2 | 112 | | |
| 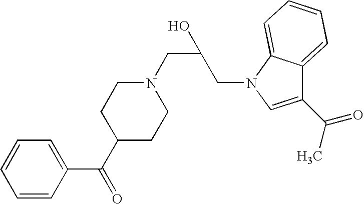 | 5.2 | 26 | | |
| 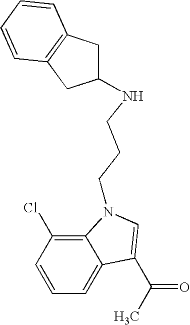 | 5.2 | 96 | | |
| 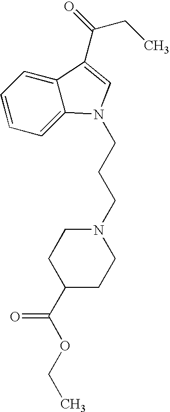 | 5.2 | 39 | | |
| 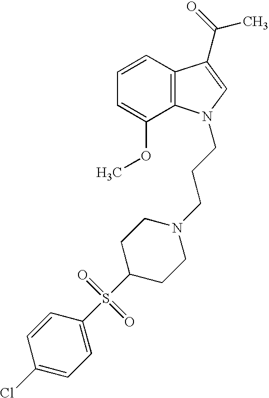 | 5.0 | 71 | | |
| 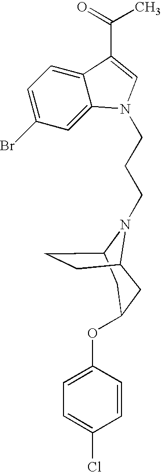 | 5.0 | 74 | | |
| 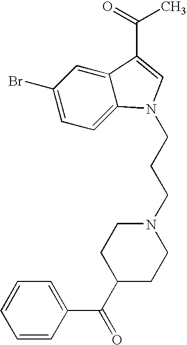 | 5.0 | 60 | | |
| 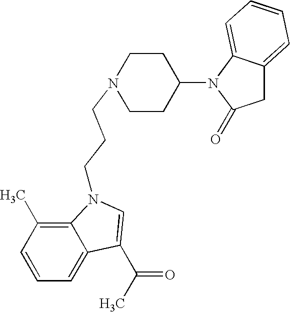 | 4.9 | 69 | | |
| 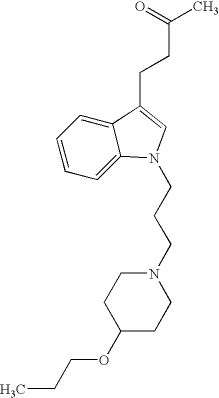 | 4.8 | 75 | | |
| 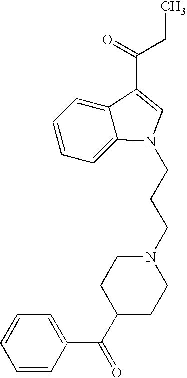 | 4.8 | 53 | | |
| 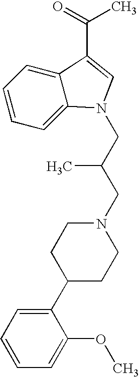 | 4.8 | 64 | | |
| 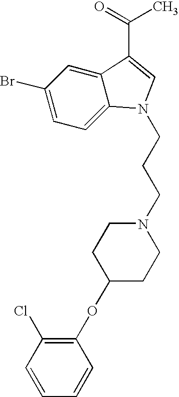 | 4.8 | 74 | | |
| 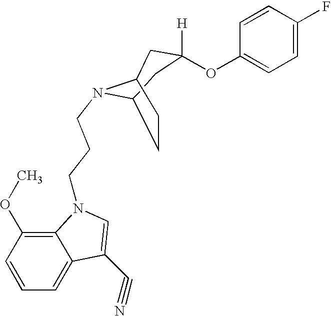 | 4.8 | 107 | 6.7 | |
| 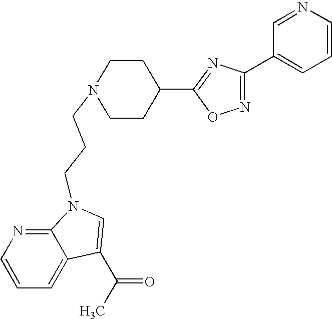 | 4.7 | 55 | | |
| 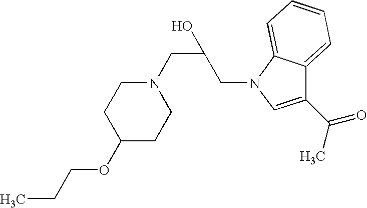 | 4.5 | 18 | | |
| 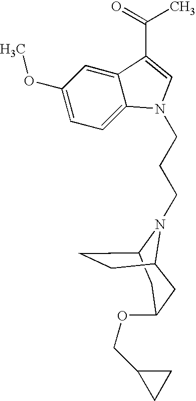 | 4.5 | 74 | | |
| 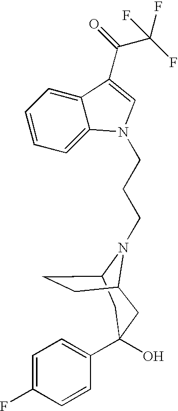 | 3.6 | 58 | | |
| 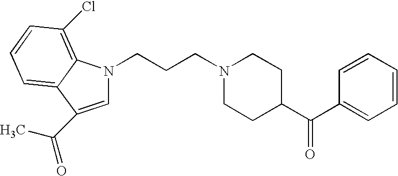 | 2.8 | 92 | | |
| 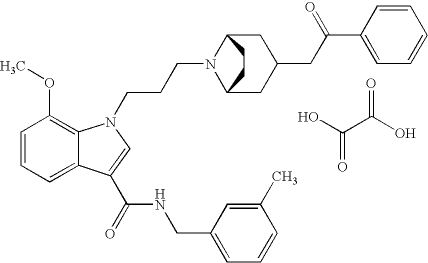 | nd | | 7.2 | |
| 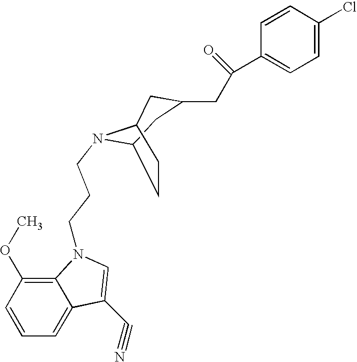 | — | 12 | 7.1 | |
| 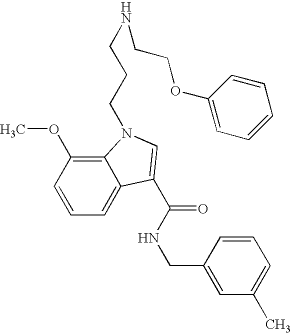 | — | 52 | 7.0 | |
| 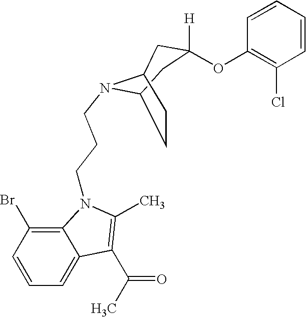 | — | 37 | 7.0 | |
| 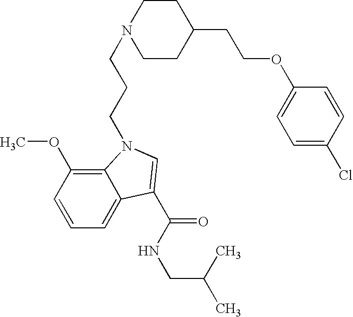 | — | 45 | 6.8 | |
| 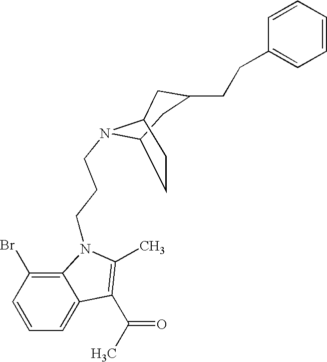 | nd | | 6.7 | |
| 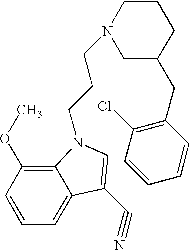 | — | 62 | 6.7 | |
| 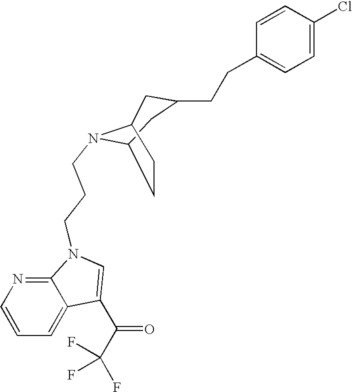 | nd | | 6.6 | |
| 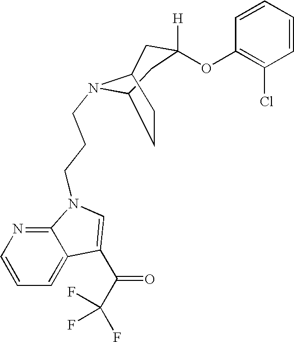 | — | 23 | 6.6 | |
| 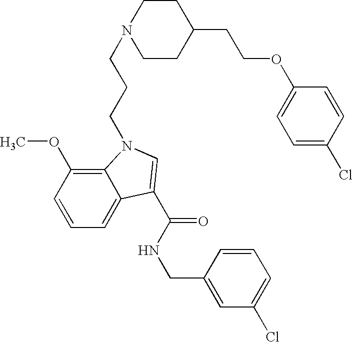 | — | 46 | 6.6 | |
| 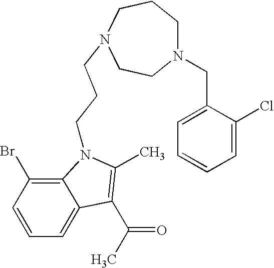 | 13 | −14 | 6.4 | |
| 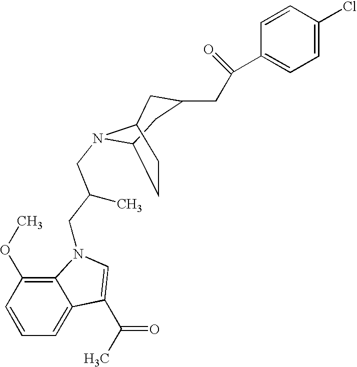 | — | 16 | 6.4 | |
| 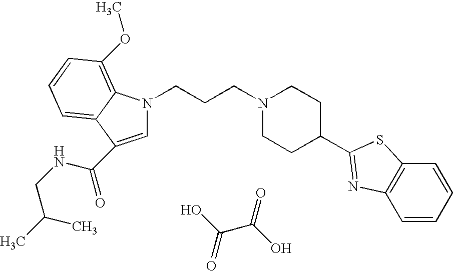 | nd | | 6.3 | |
| 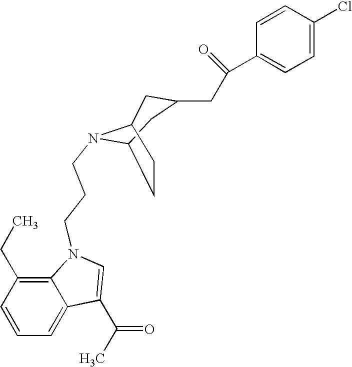 | nd | | 6.3 | |
| 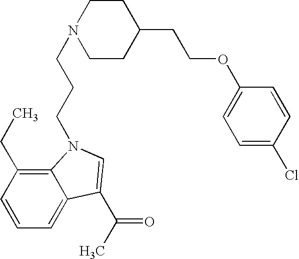 | — | 76 | 6.3 | |
| 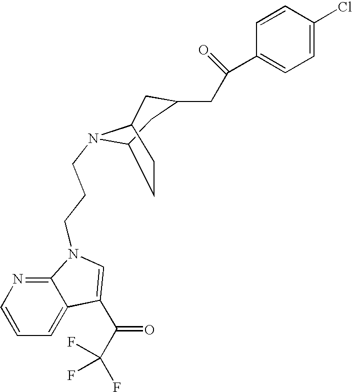 | — | 10 | 6.2 | |
| 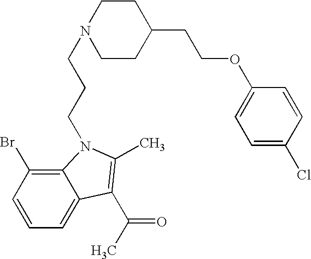 | nd | | 6.2 | |
| 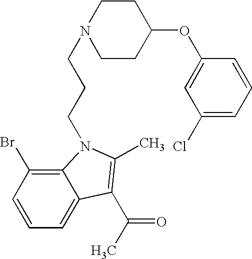 | — | 35 | 6.2 | |
| 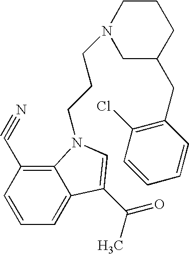 | — | 22 | 6.1 | |
| 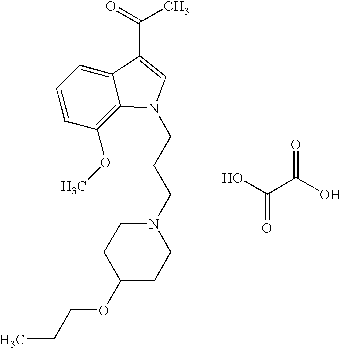 | nd | | 5.9 | |
| 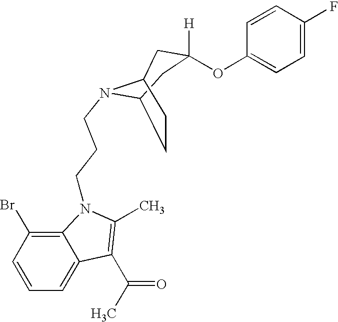 | — | 1 | 5.8 | |
| 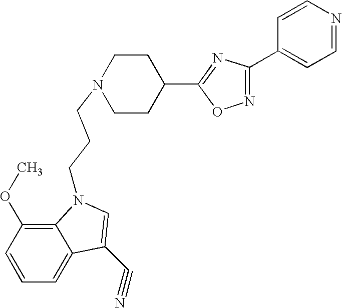 | — | 40 | — | |
| 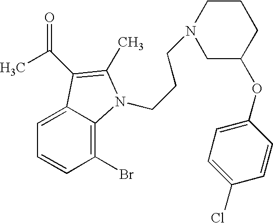 | — | 24 | — | |
| 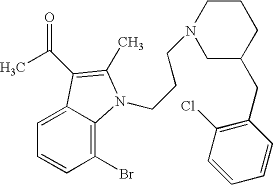 | — | 5 | — | |
| 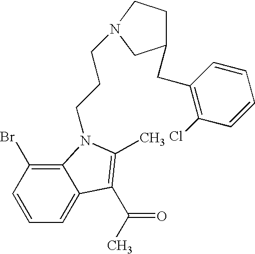 | — | 38 | — | |
| 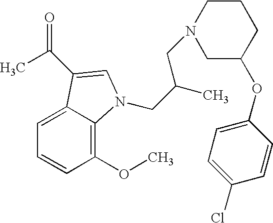 | — | −1 | — | |
| 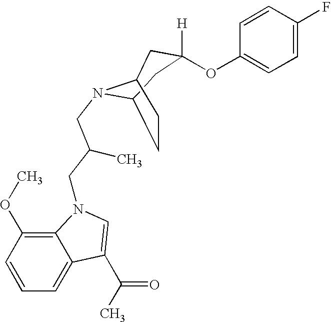 | — | 44 | — | |
| 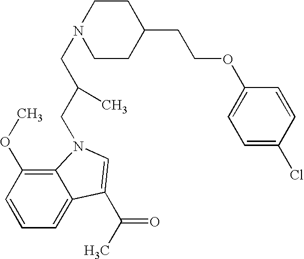 | — | 22 | — | |
| 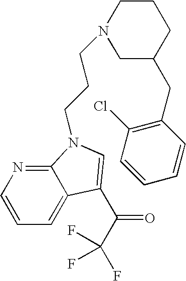 | — | 27 | — | |
| 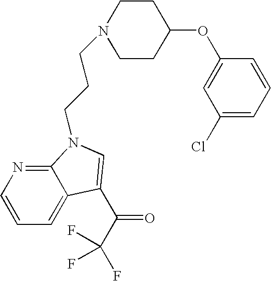 | — | 23 | — | |
| 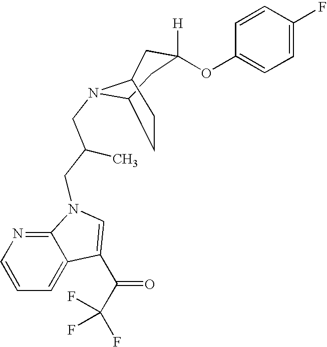 | — | 35 | — | |
| 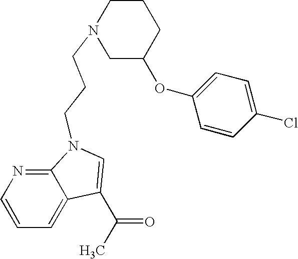 | — | 53 | — | |
| 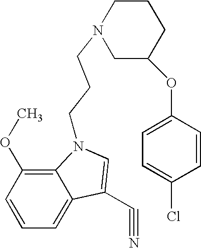 | — | 48 | — | |
| 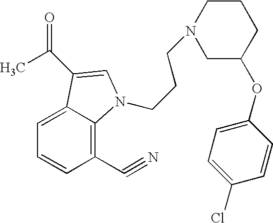 | — | 31 | — | |
| 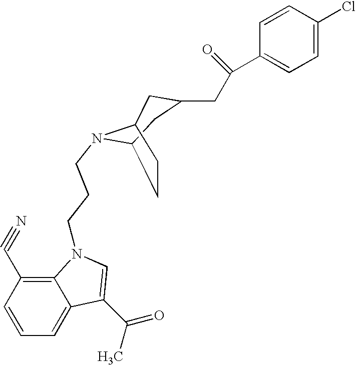 | 5— | 31 | — | |
| 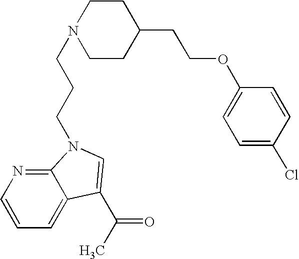 | 5nd | | — | |
| 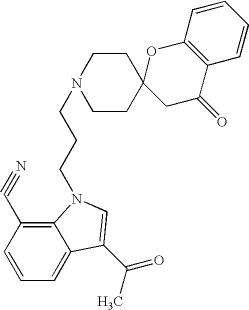 | 5nd | | — | |
| 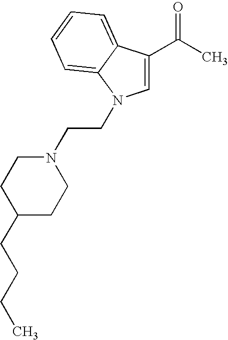 | 5— | 23 | | |
| 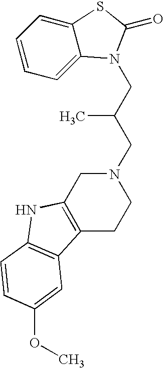 | 5— | −10 | | |
| 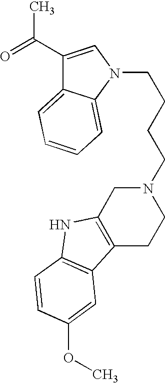 | — | 9 | | |
| 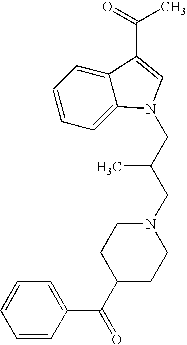 | — | 2 | | |
| 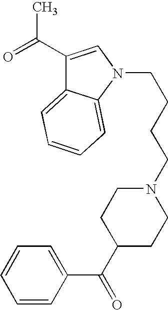 | — | 38 | | |
| 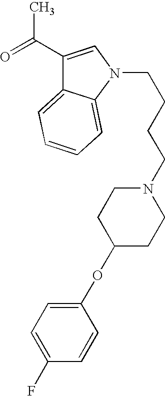 | — | 41 | | |
| 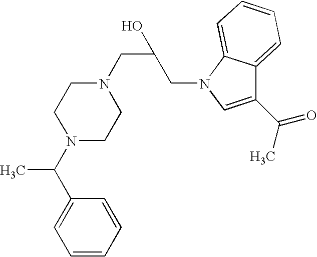 | — | 23 | | |
| 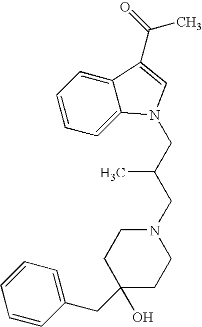 | — | −3 | | |
| 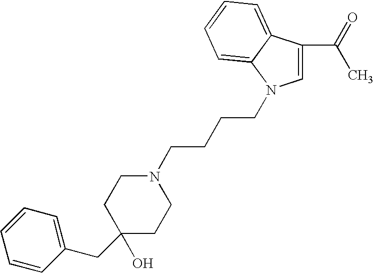 | — | 24 | | |
| 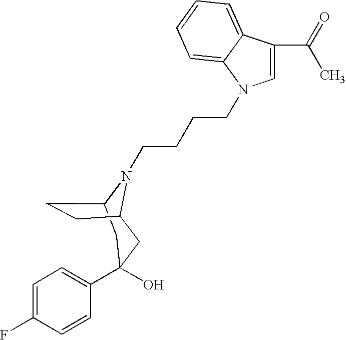 | — | 2 | | |
| 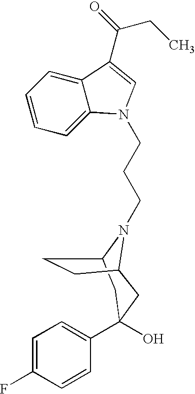 | — | 18 | | |
| 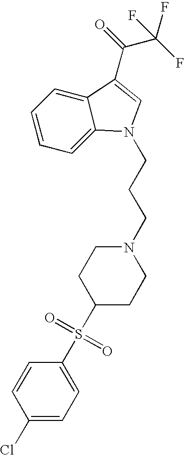 | — | 6 | | |
| 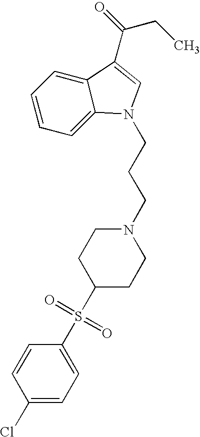 | — | 1 | | |
| 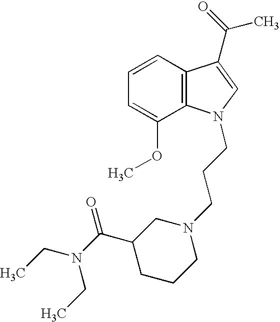 | — | 16 | | |
| 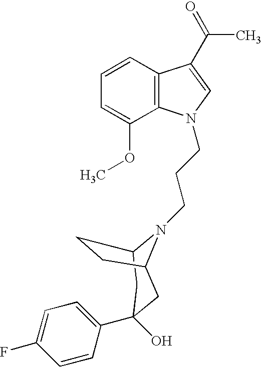 | — | 3 | | |
| 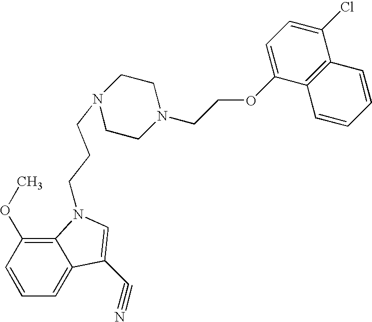 | — | 21 | | |
| 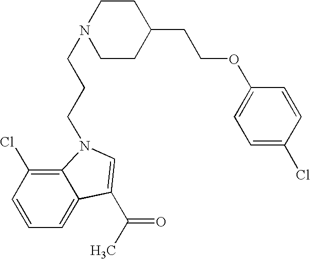 | — | 53 | | |
| 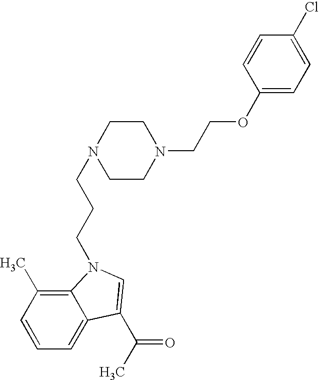 | — | 53 | | |
| 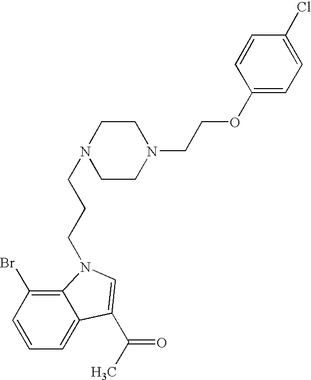 | — | 12 | | |
| 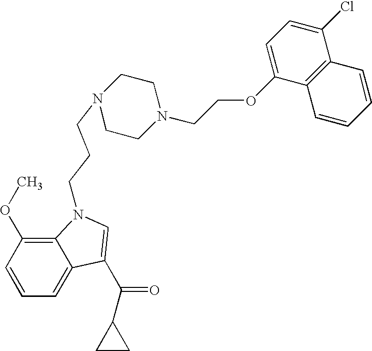 | — | 44 | | |
| 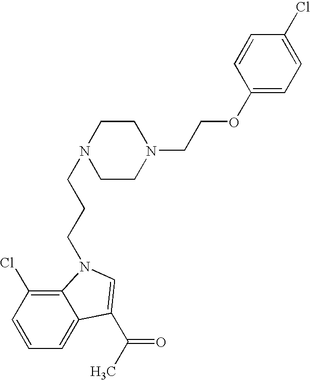 | — | 32 | | |
| 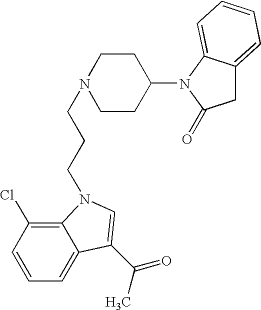 | — | 28 | | |
| 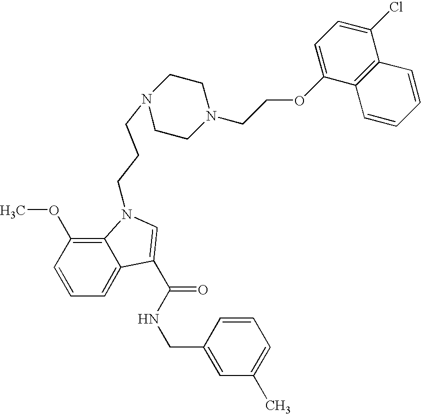 | — | 2 | | |
| 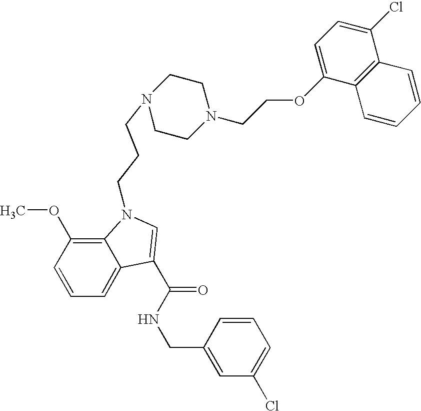 | — | 59 | | |
| 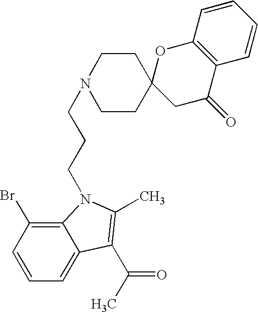 | — | 8 | | |
| 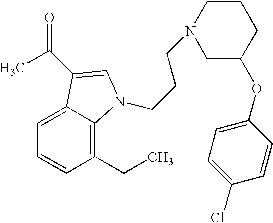 | — | 13 | | |
| 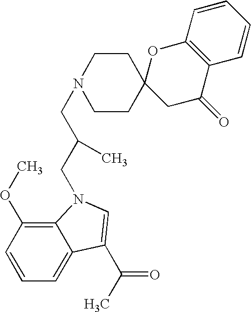 | — | 55 | | |
| 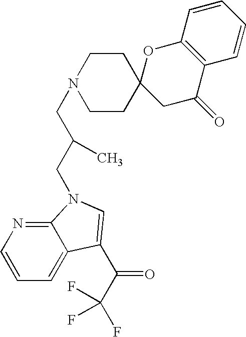 | — | 26 | | |
| 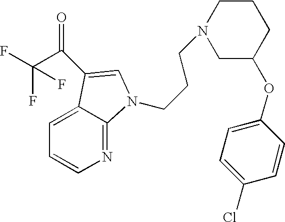 | — | 2 | | |
| 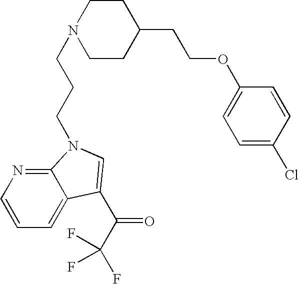 | — | 37 | | |
| 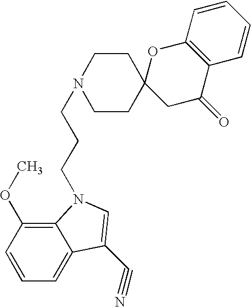 | — | 41 | | |
| 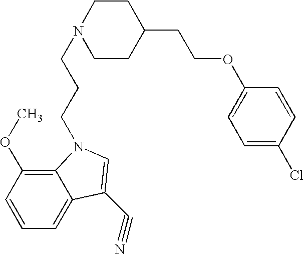 | — | −3 | | |
| 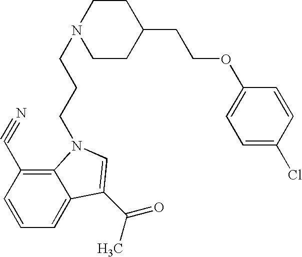 | — | 27 | | |
| 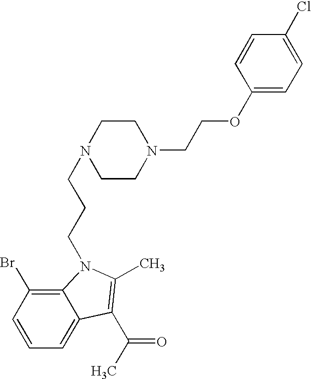 | — | −13 | | |
| 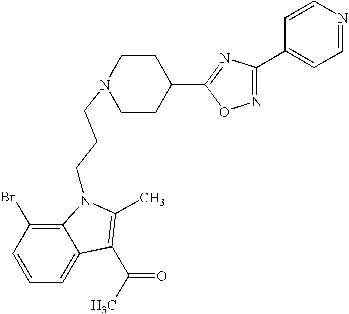 | — | 25 | | |
| 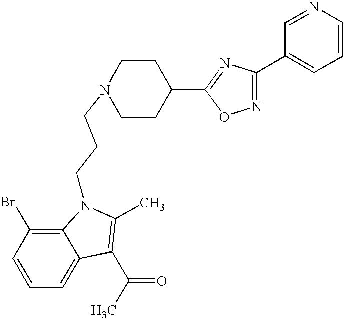 | — | 12 | | |
| 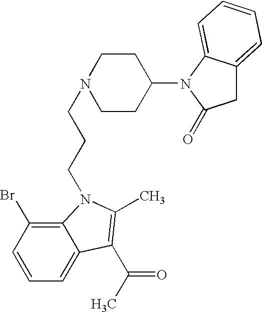 | — | 12 | | |
| 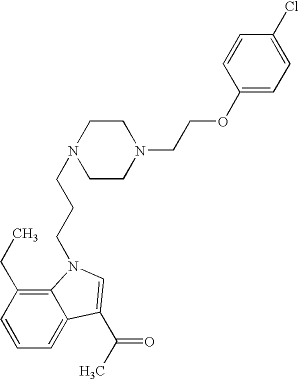 | — | 24 | | |
| 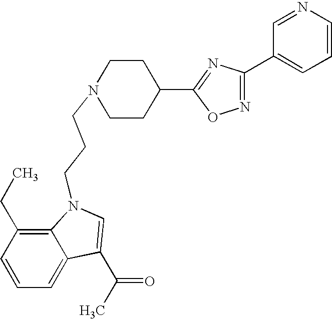 | — | 61 | | |
| 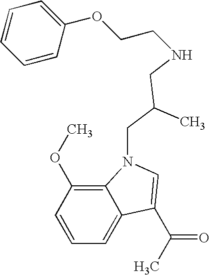 | — | 29 | | |
| 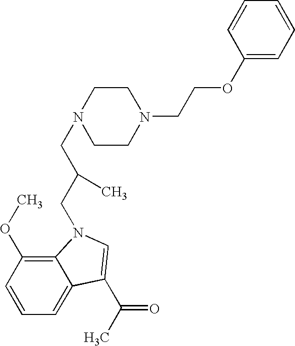 | — | −7 | | |
| 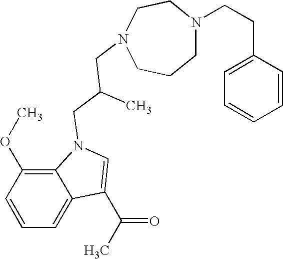 | — | 31 | | |
| 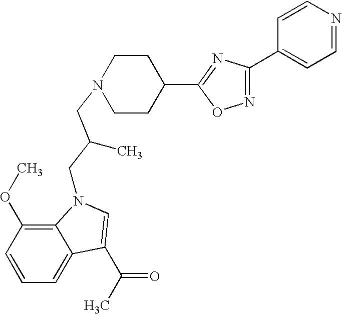 | — | 78 | | |
| 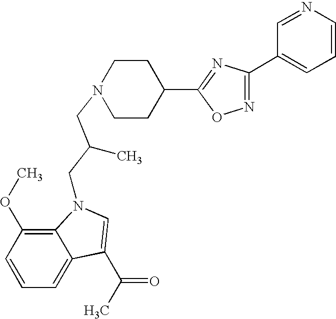 | — | 83 | | |
| 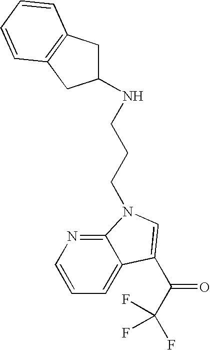 | — | −3 | | |
| 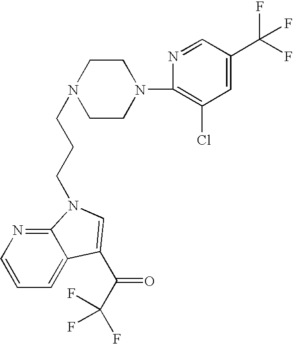 | — | 27 | | |
| 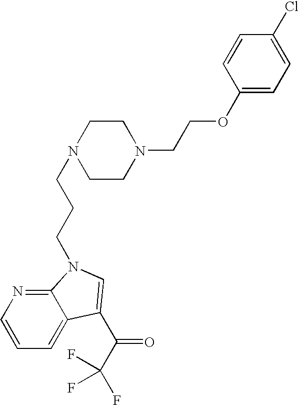 | — | 38 | | |
| 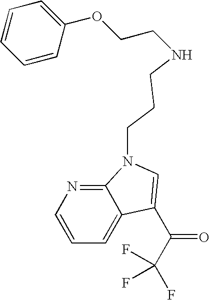 | — | 26 | | |
| 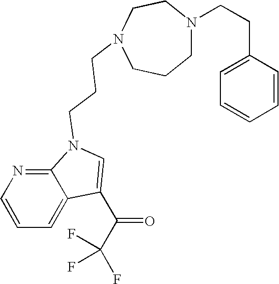 | — | 51 | | |
| 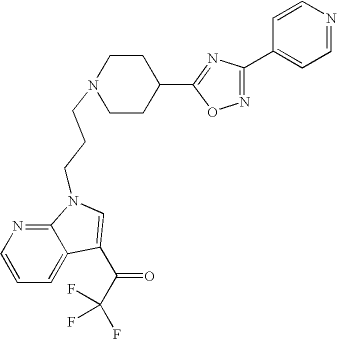 | — | 31 | | |
| 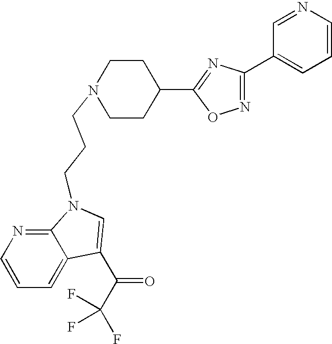 | — | 72 | | |
| 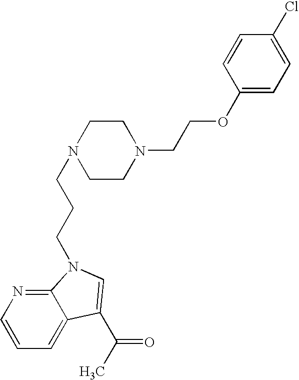 | — | 62 | | |
| 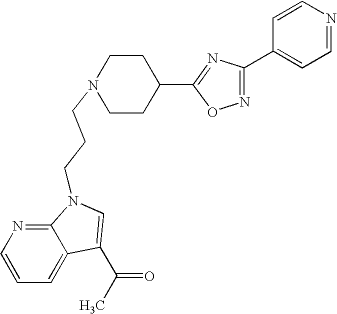 | — | 88 | | |
| 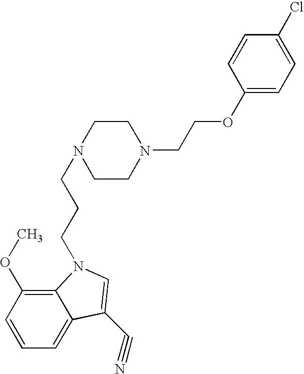 | — | −23 | | |
| 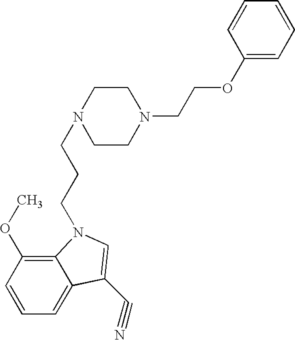 | — | 42 | | |
| 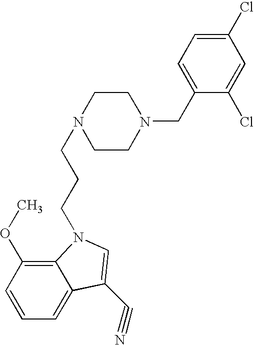 | — | 30 | | |
| 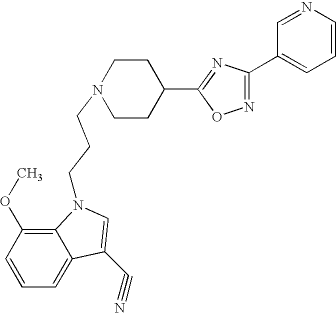 | — | 23 | | |
| 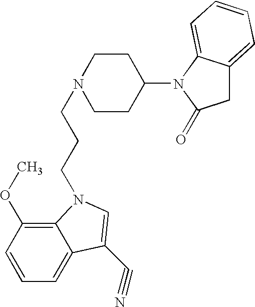 | — | 33 | | |
| 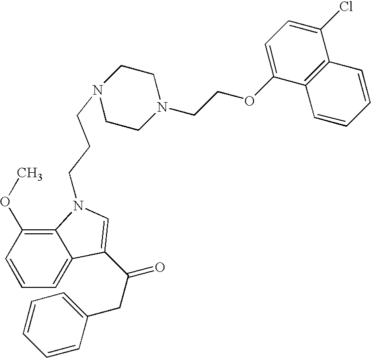 | — | 46 | | |
| 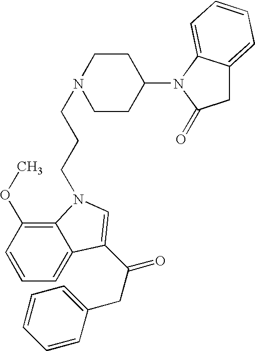 | — | 24 | | |
| 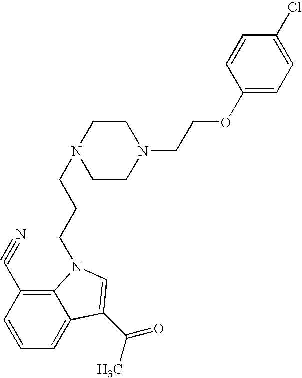 | — | 4 | | |
| 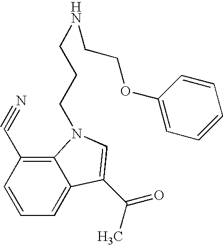 | — | 11 | | |
| 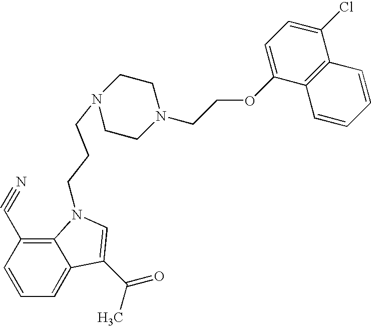 | — | 53 | | |
| 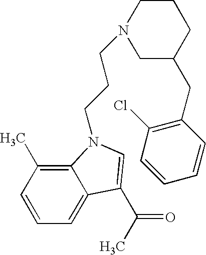 | — | 38 | | |
| 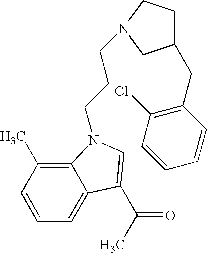 | — | 47 | | |
| 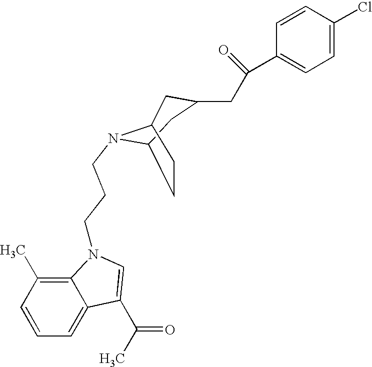 | — | 19 | | |
| 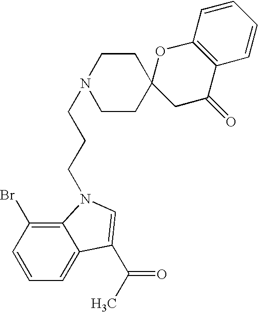 | — | 3 | | |
| 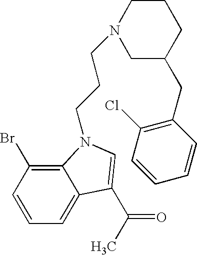 | — | −18 | | |
| 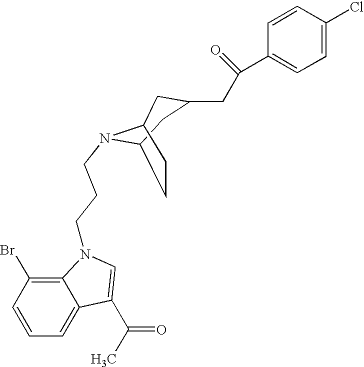 | — | 33 | | |
| 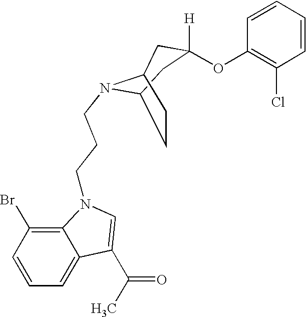 | — | 65 | | |
| 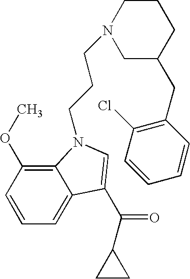 | — | 28 | | |
| 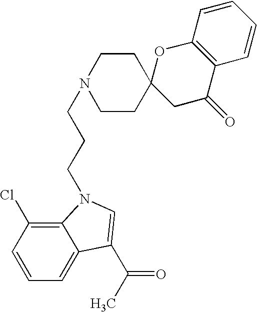 | — | 40 | | |
| 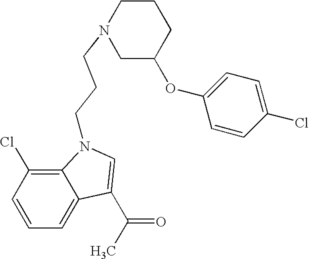 | — | 7 | | |
| 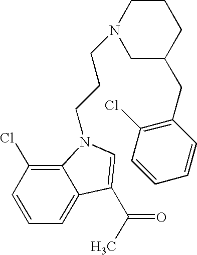 | — | 47 | | |
| 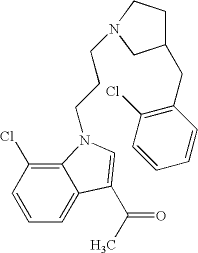 | — | 37 | | |
| 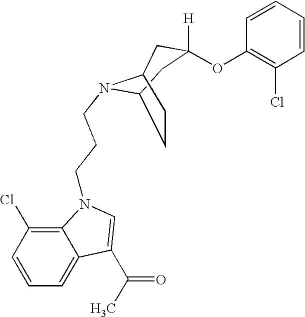 | — | 60 | | |
| 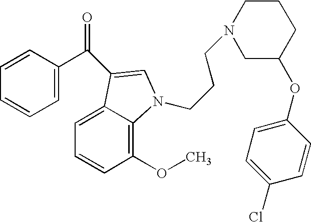 | — | 48 | | |
| 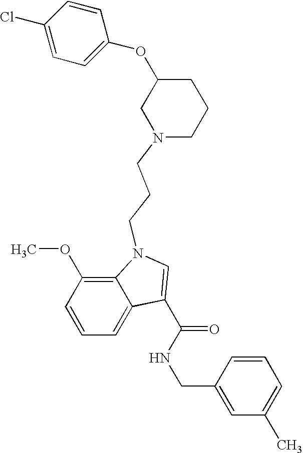 | — | 14 | | |
| 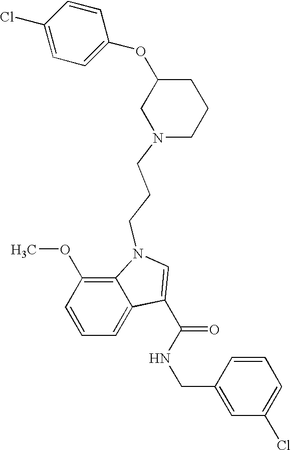 | — | 31 | | |
| 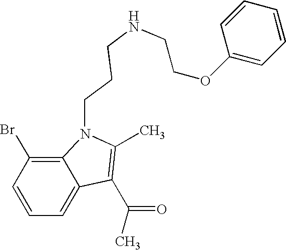 | — | 5 | | |
| 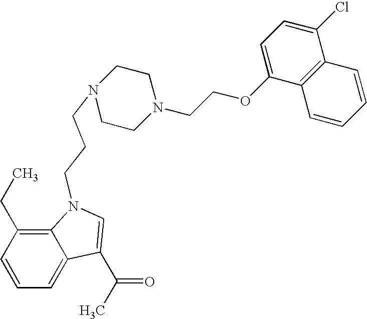 | — | 29 | | |
| 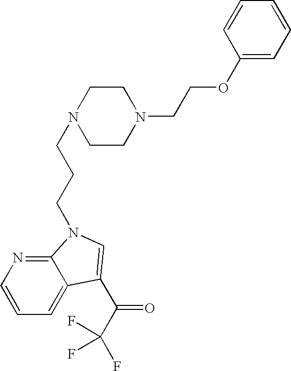 | — | 27 | | |
| 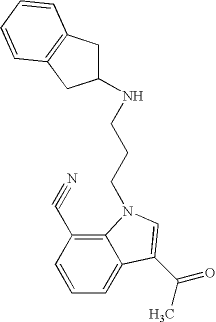 | — | 6 | | |
| 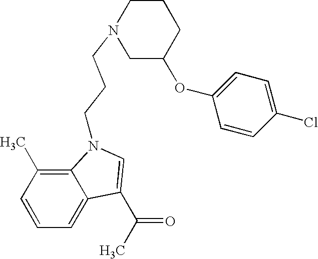 | — | −2 | | |
| 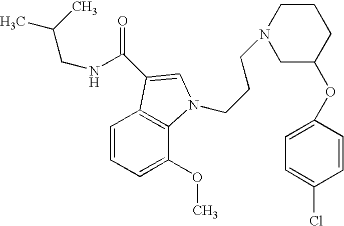 | — | 9 | | |
| 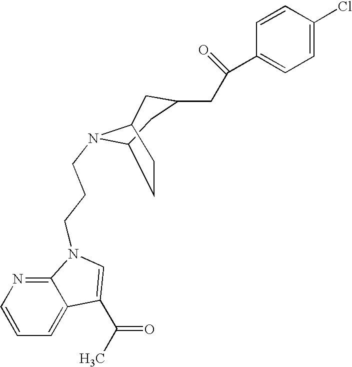 | — | 11 | | |
| 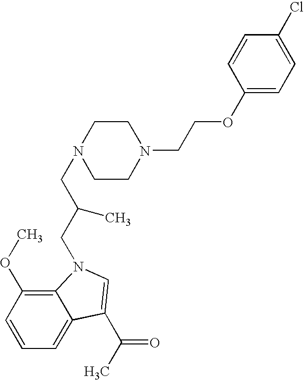 | nd | | | |
| 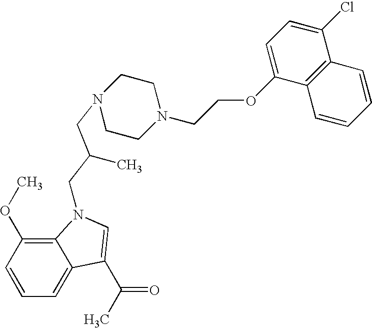 | nd | | | |
| 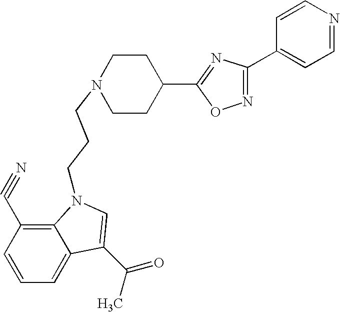 | nd | | | |
| 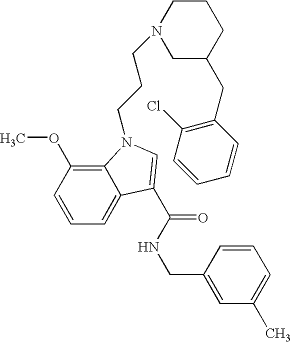 | nd | | | |
| 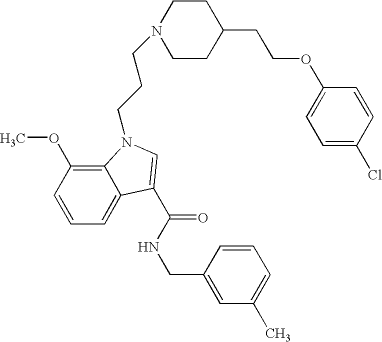 | nd | | | |
| 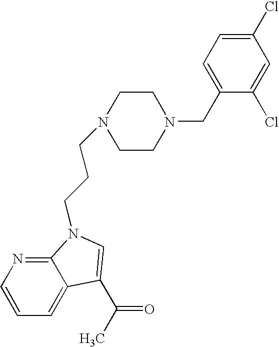 | nd | | | |

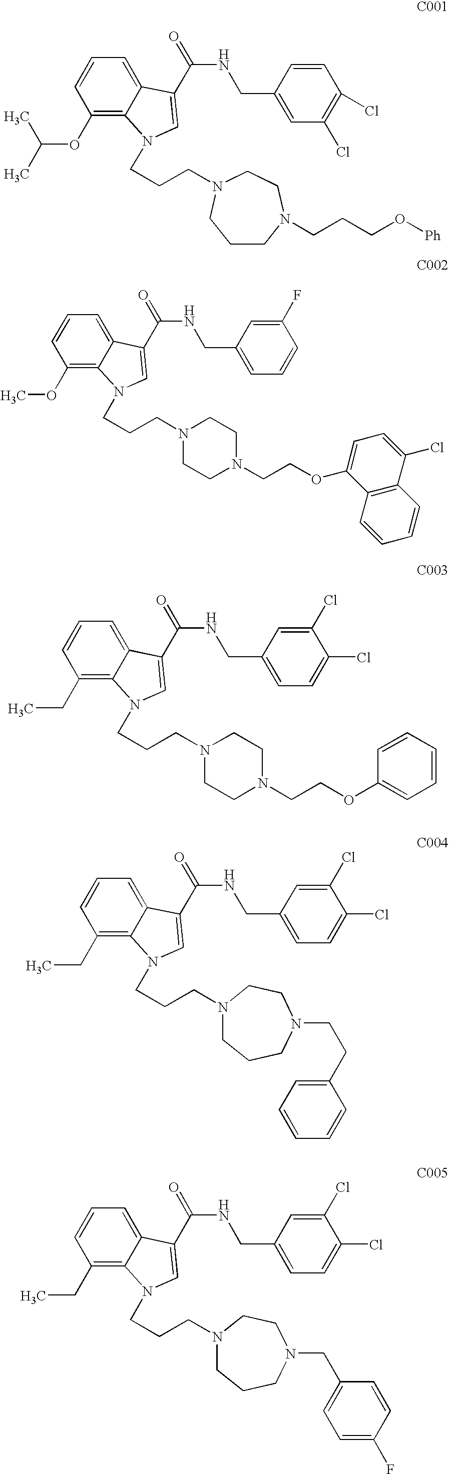

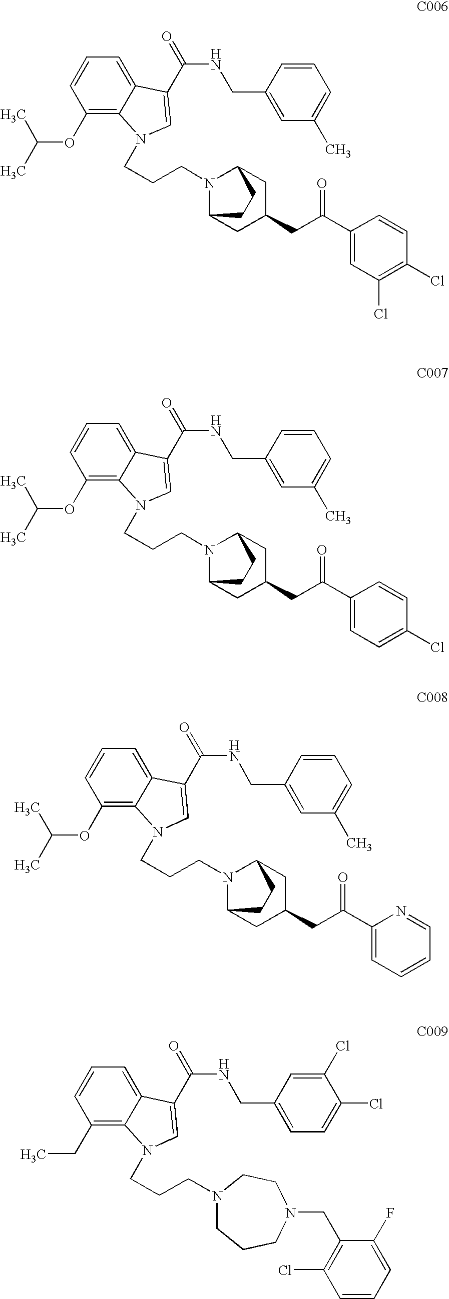

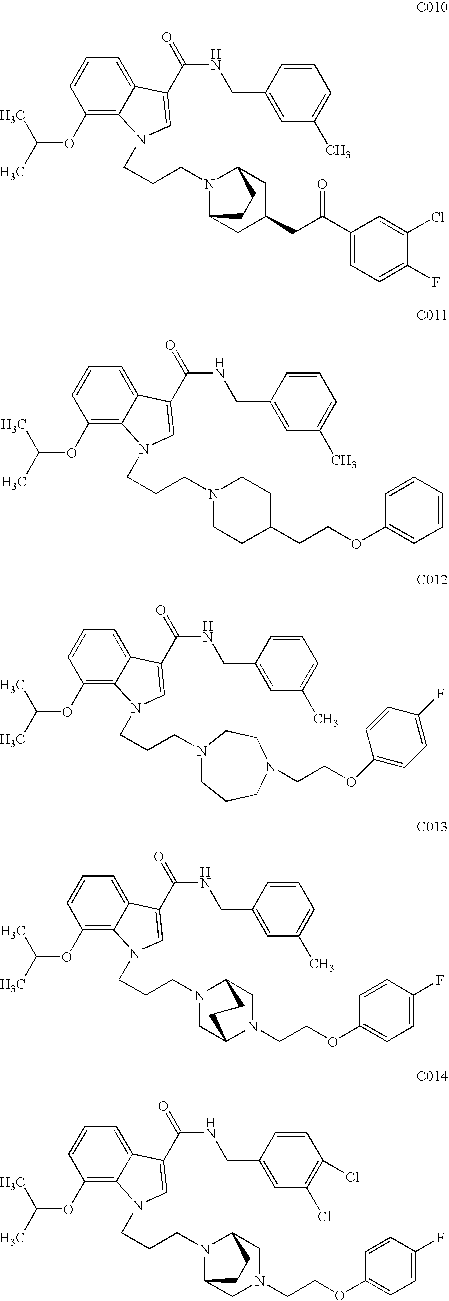

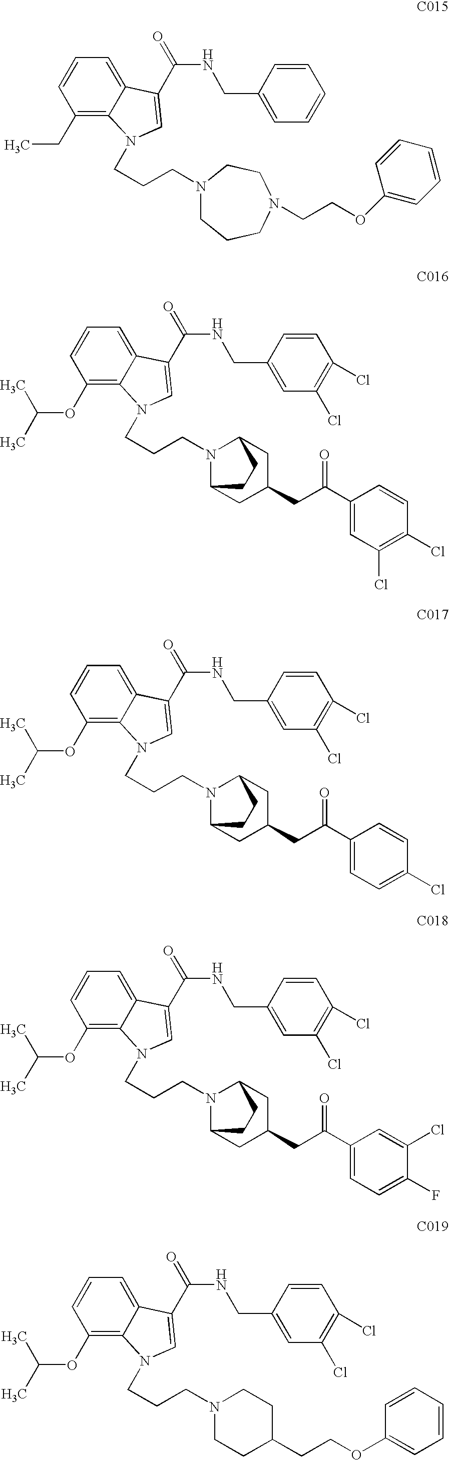

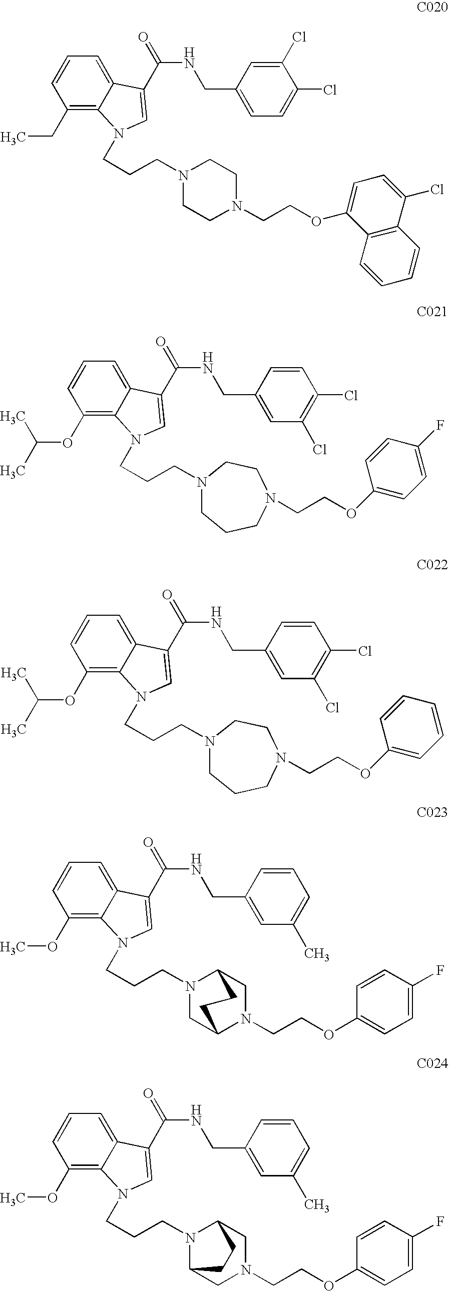

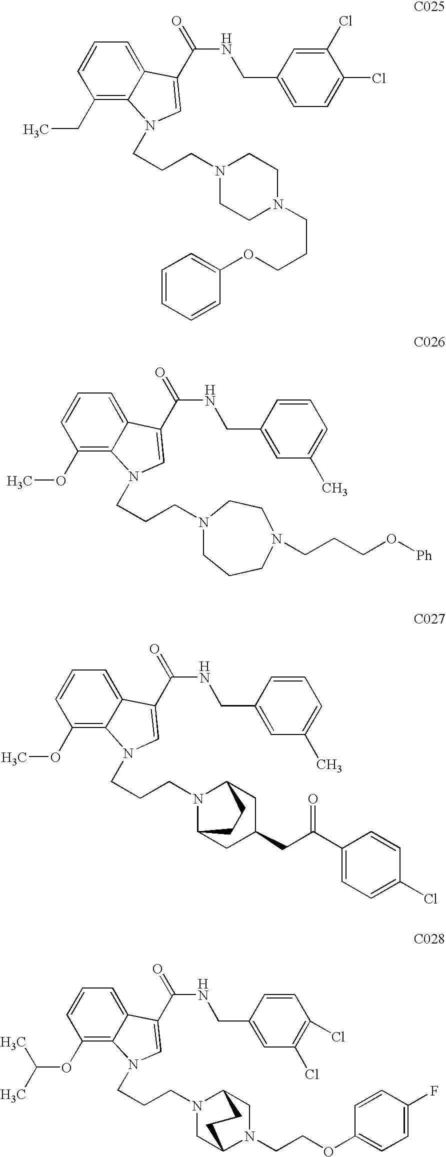

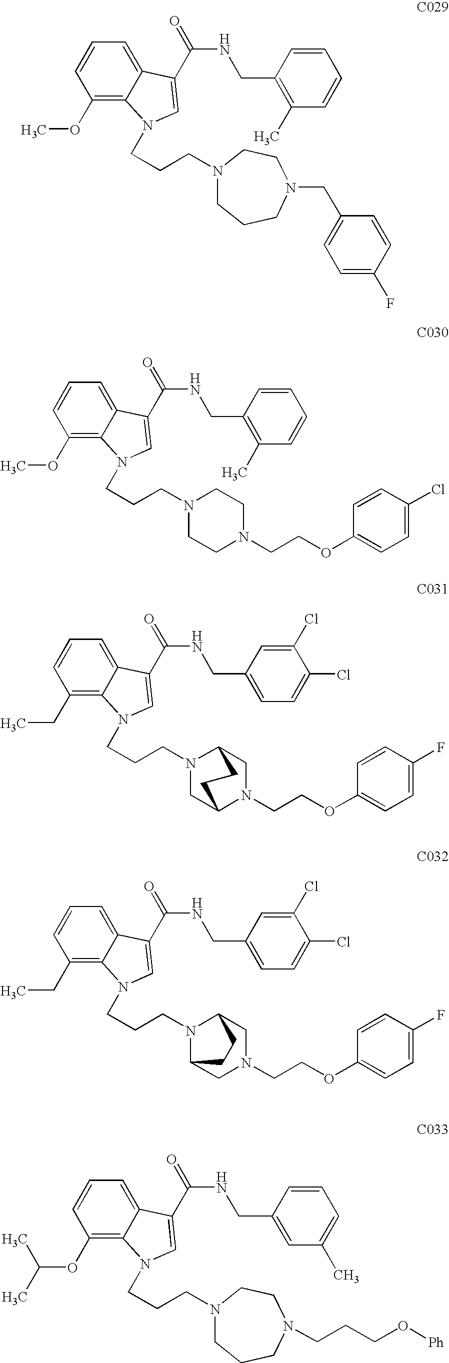

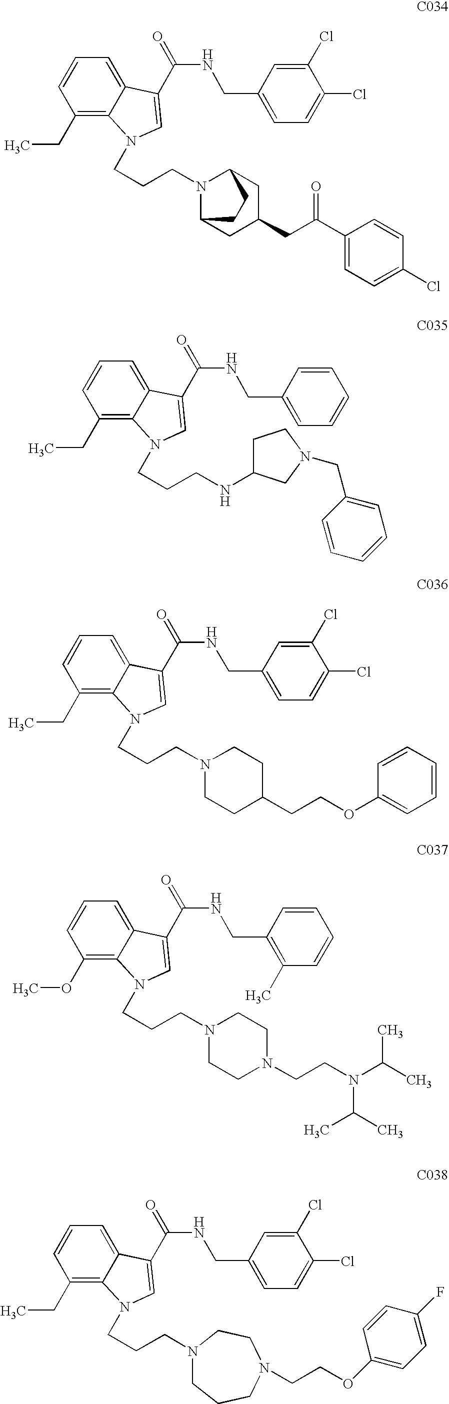

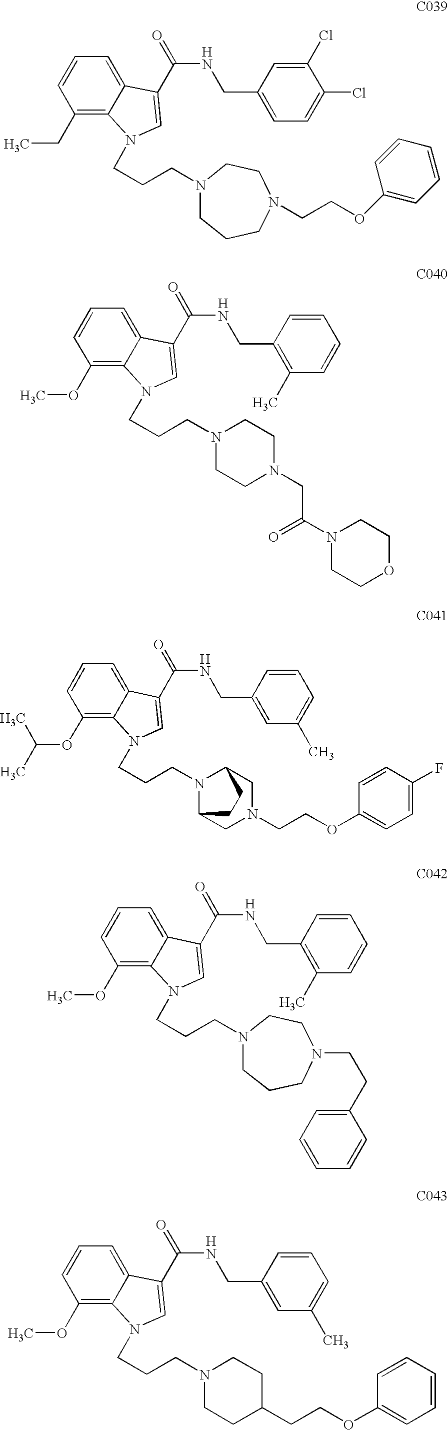

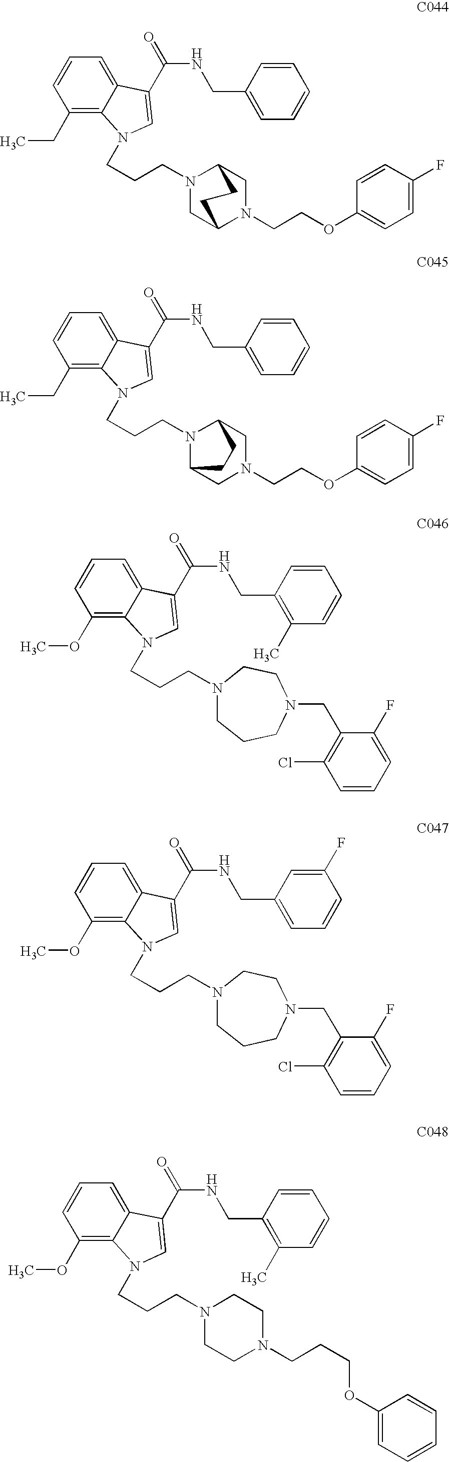

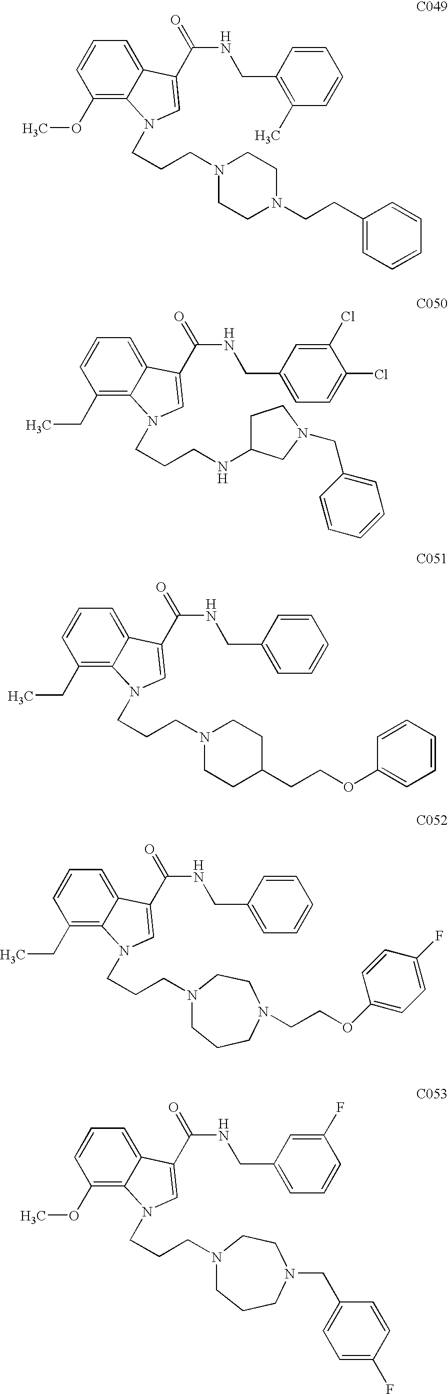

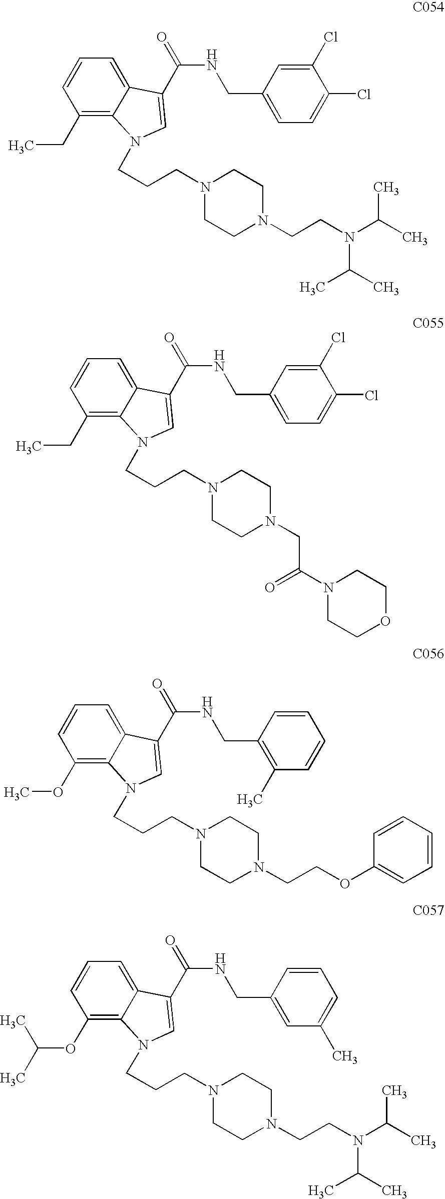

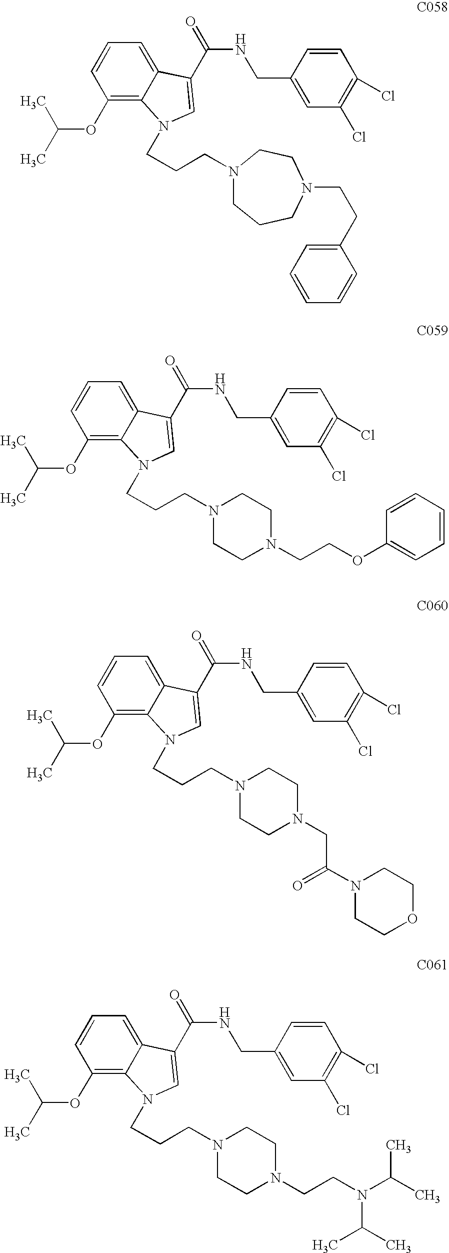

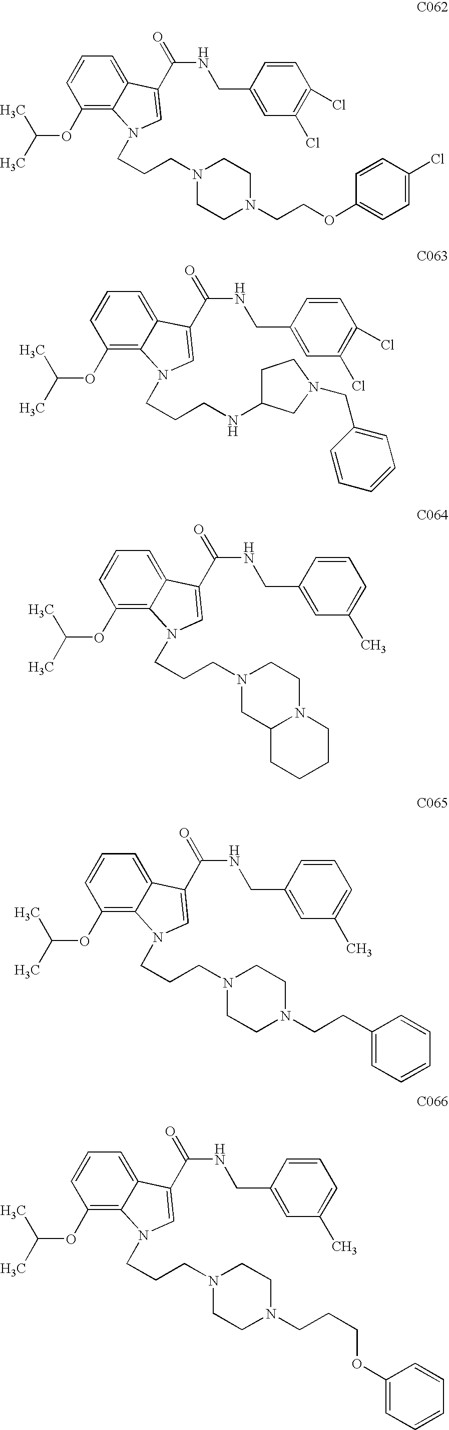

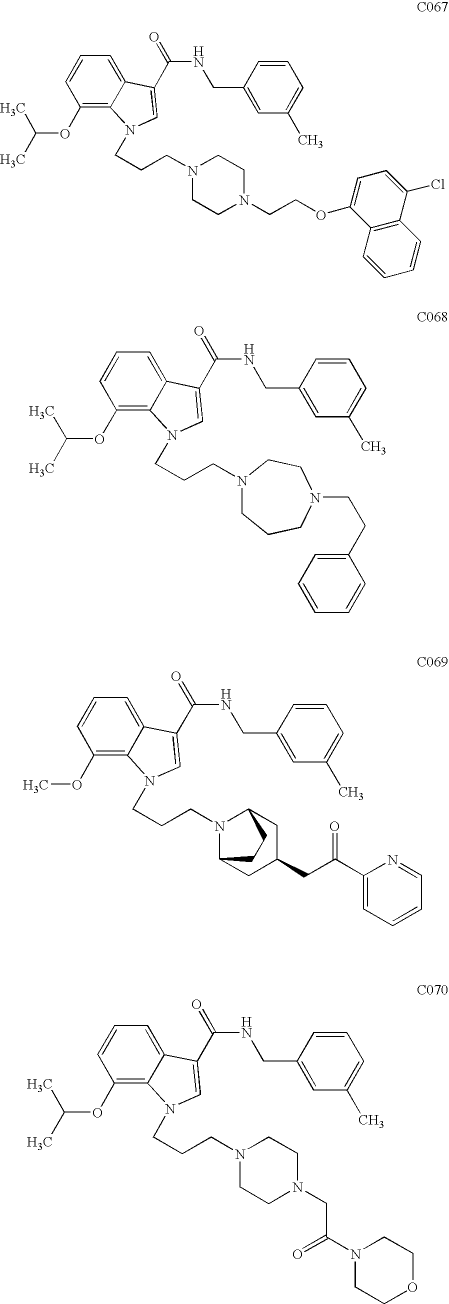

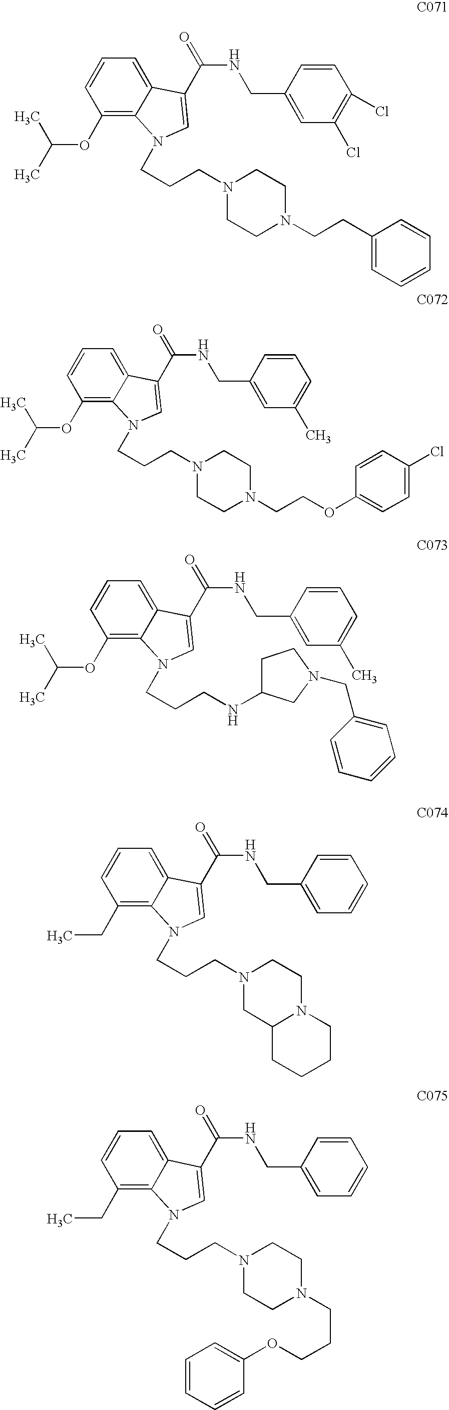

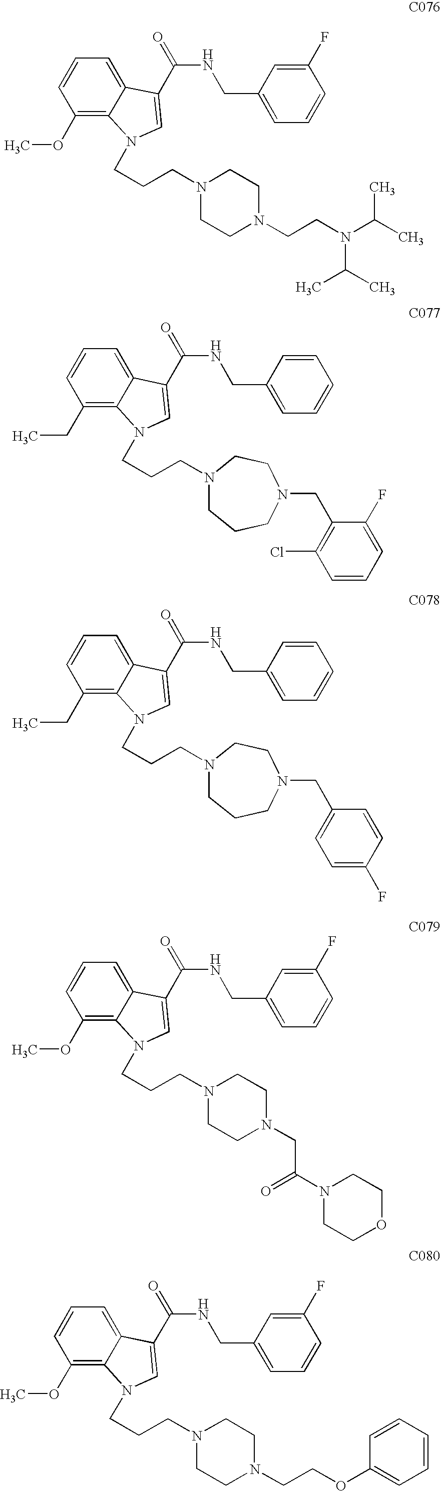

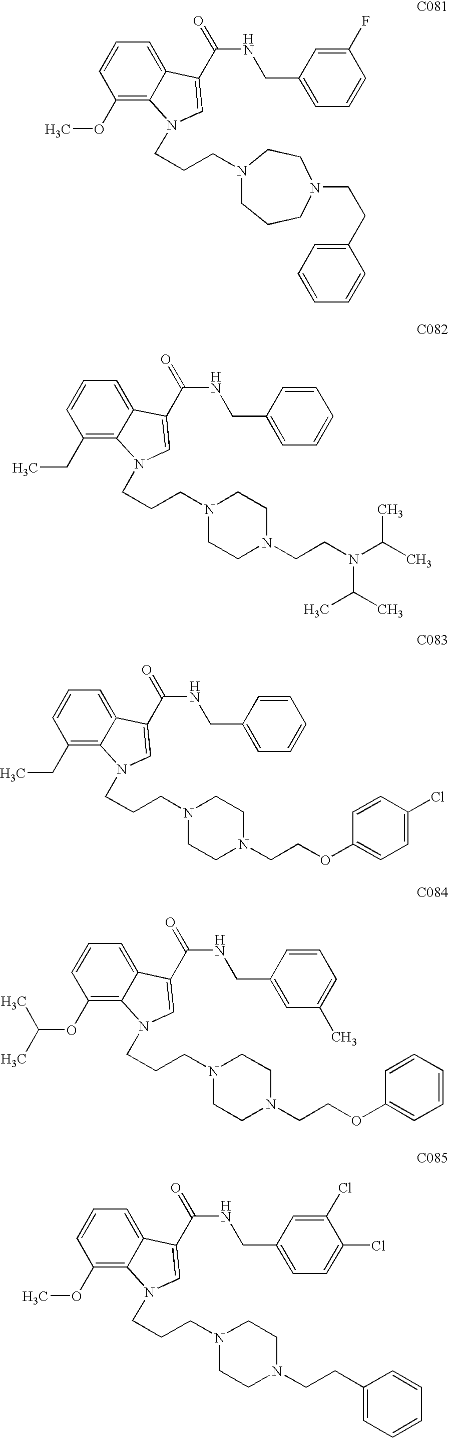

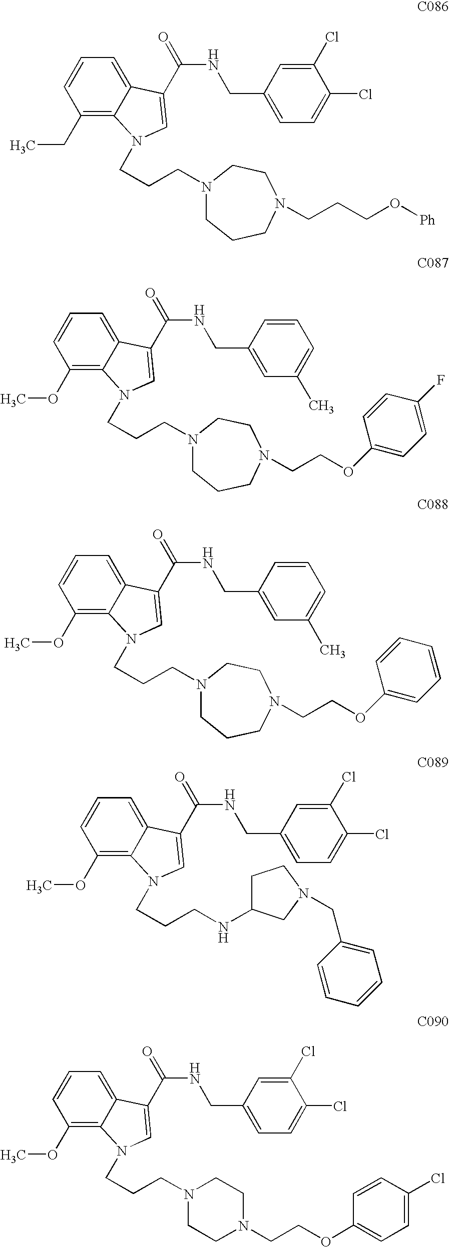

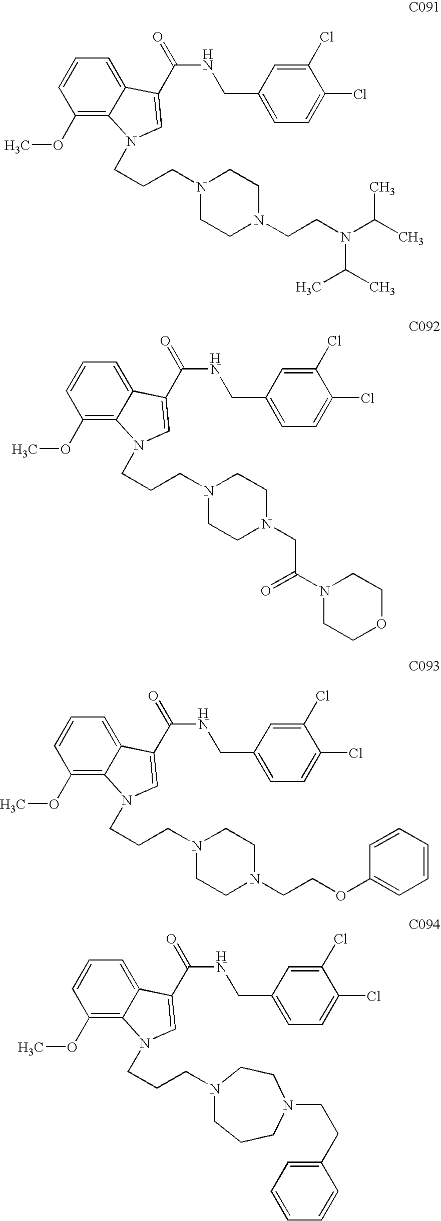

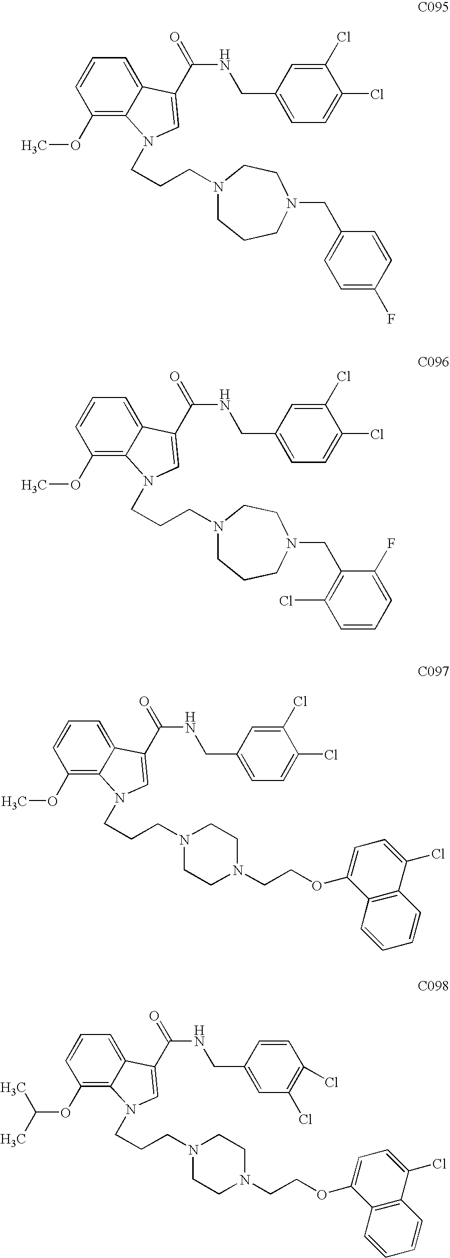

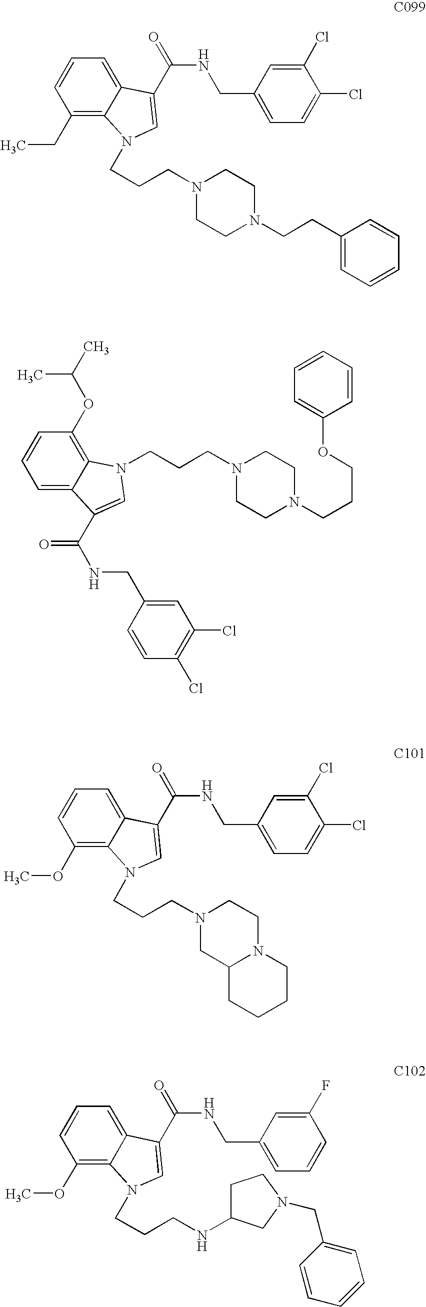

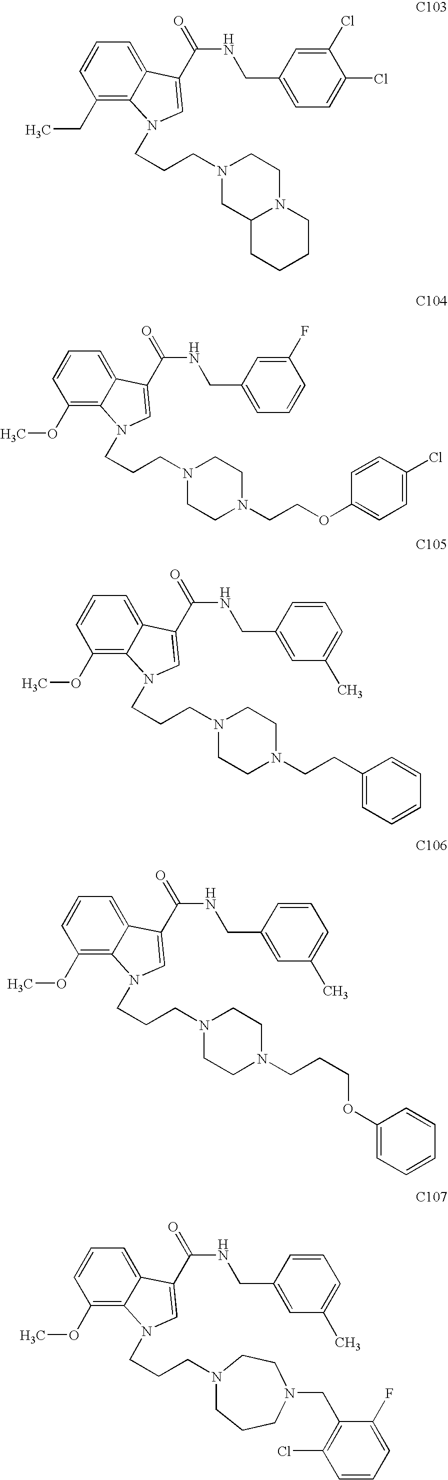

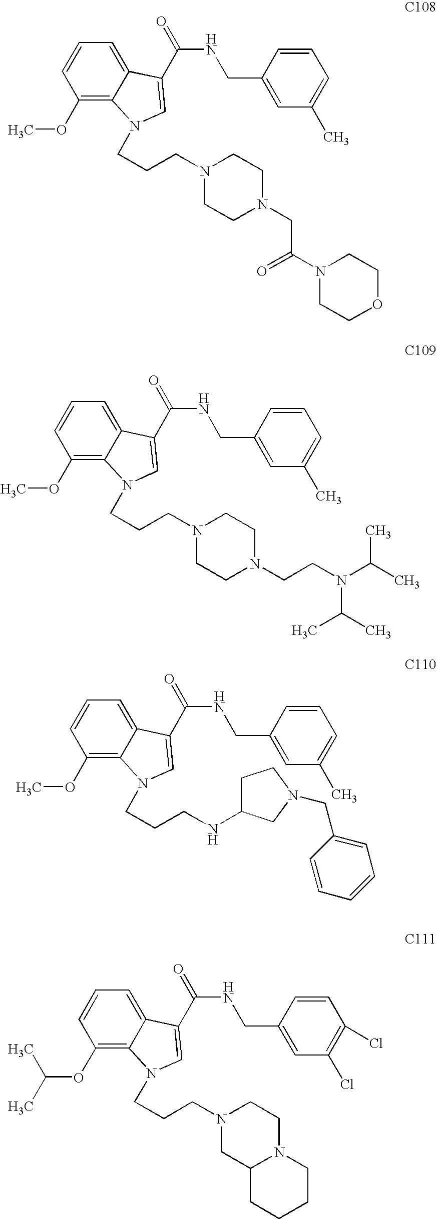

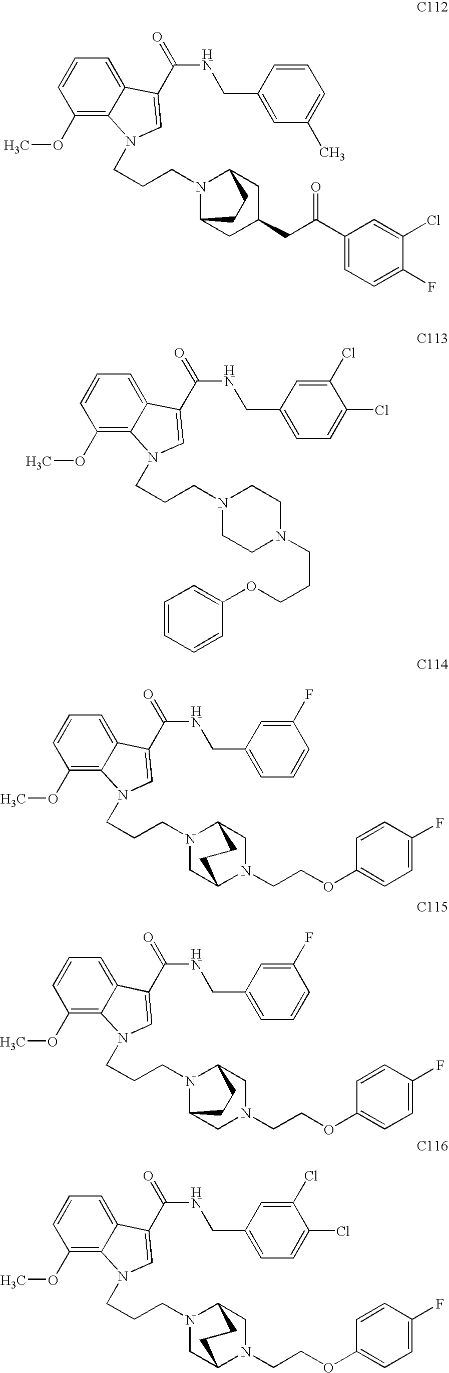

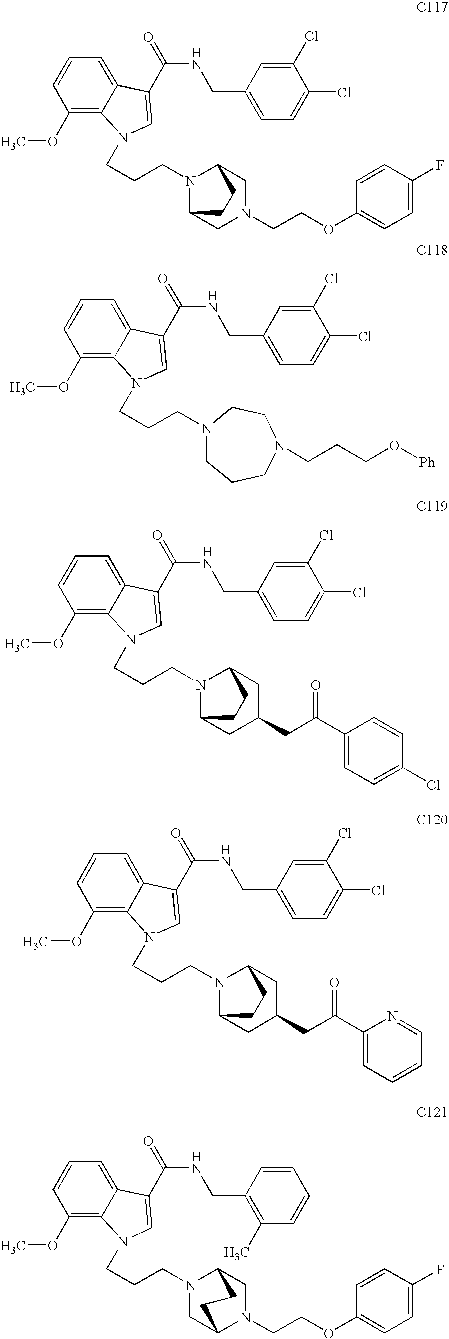

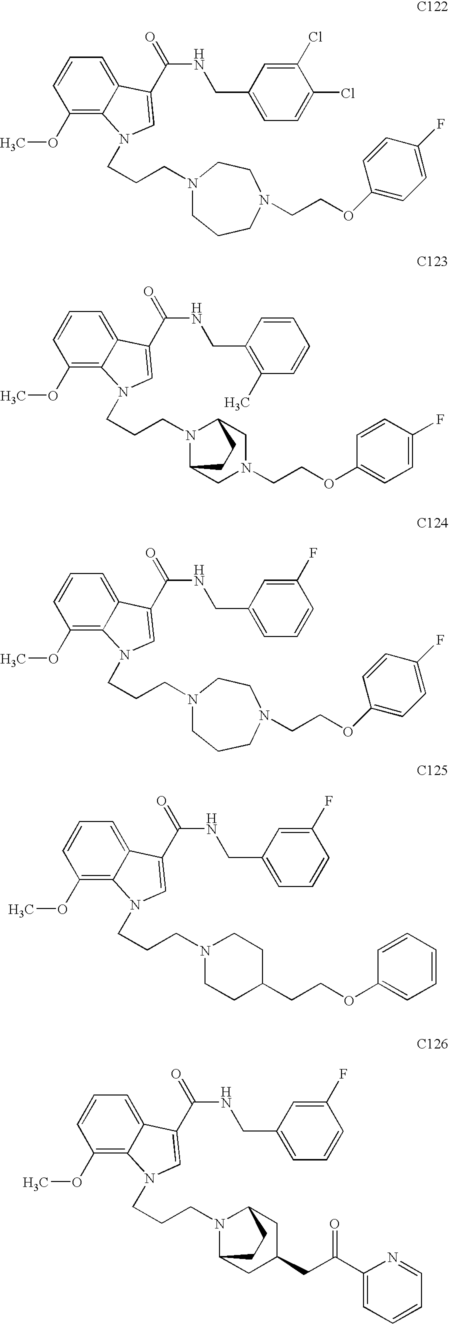

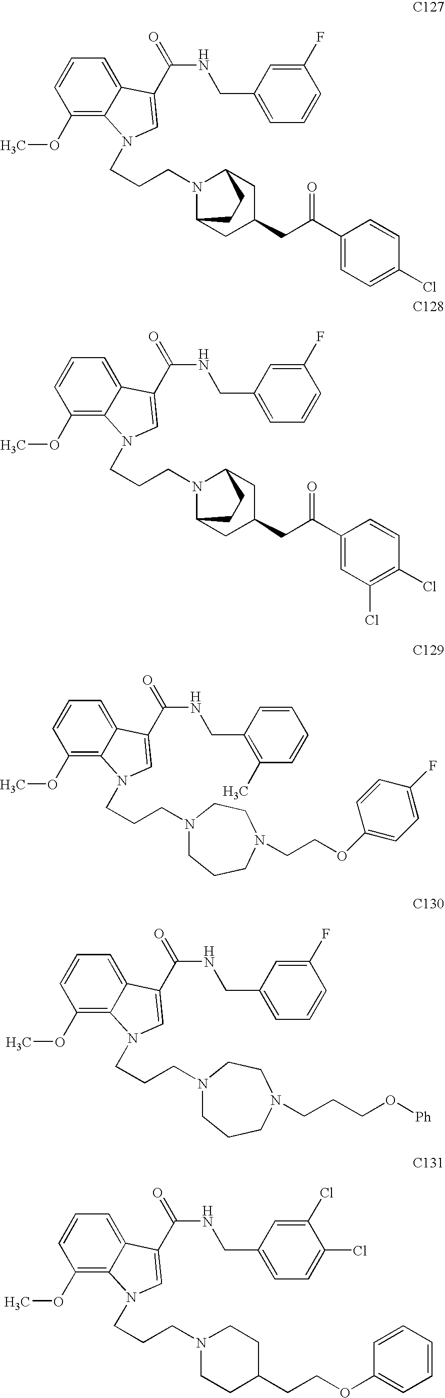

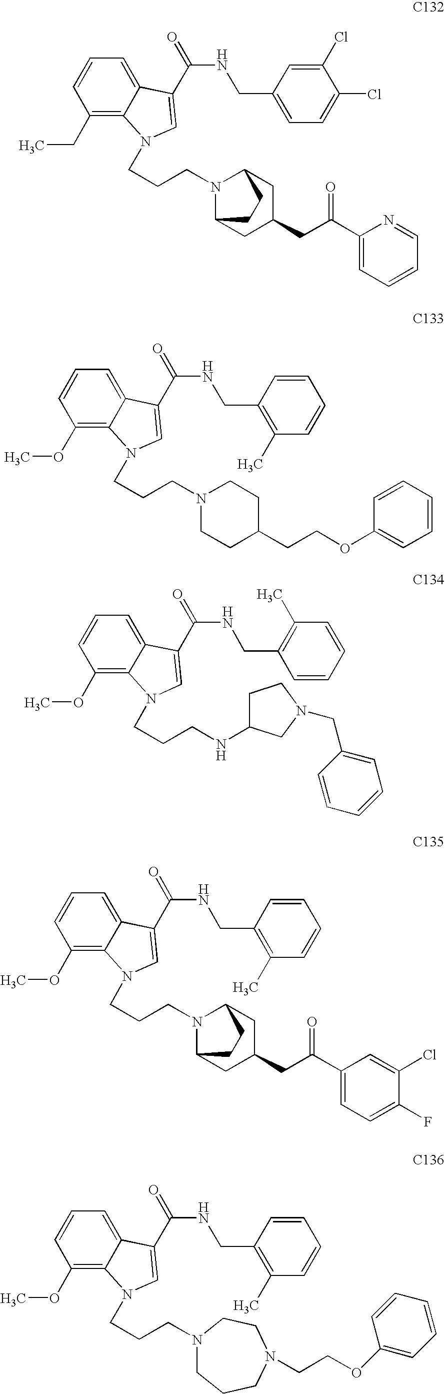

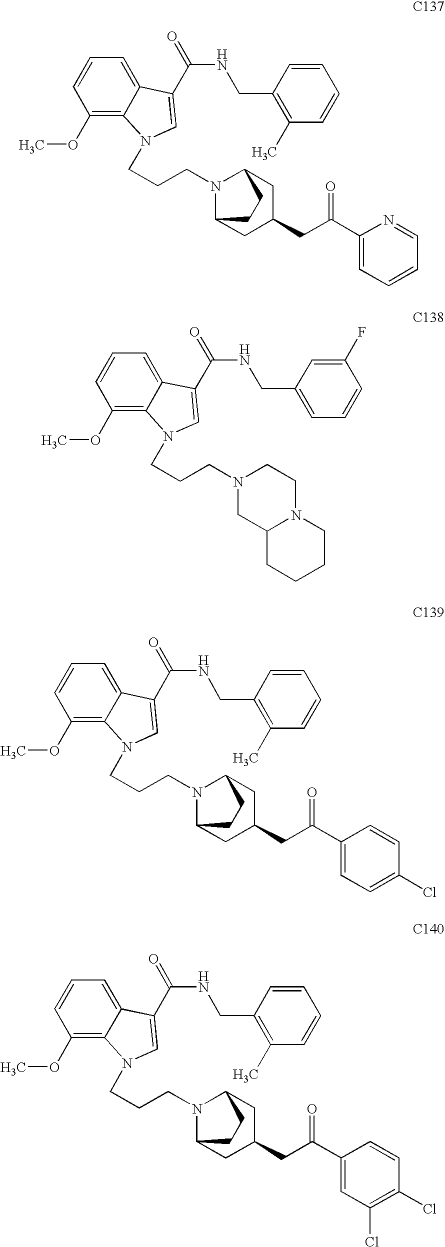

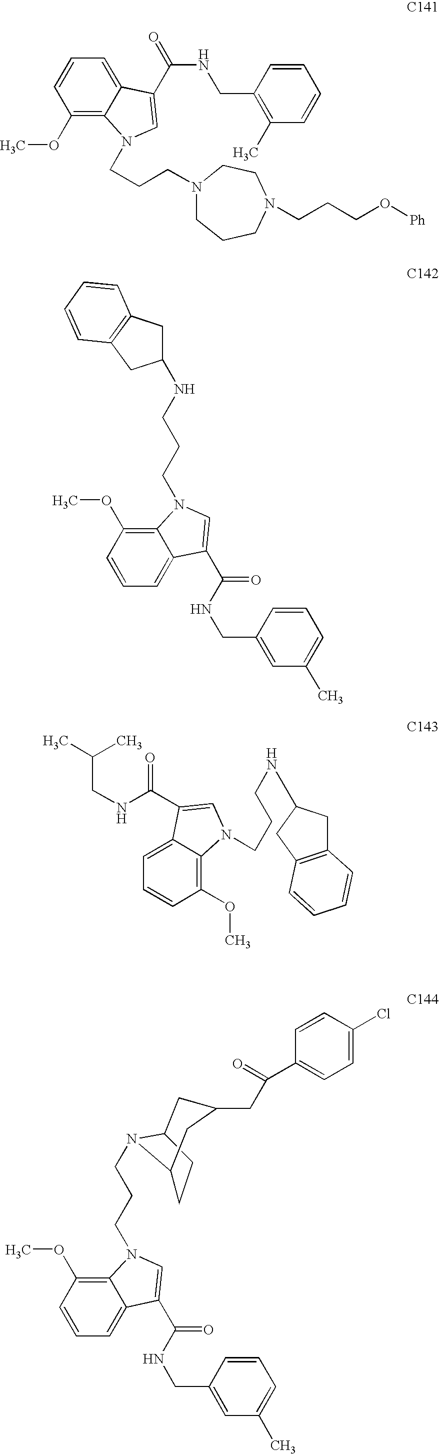

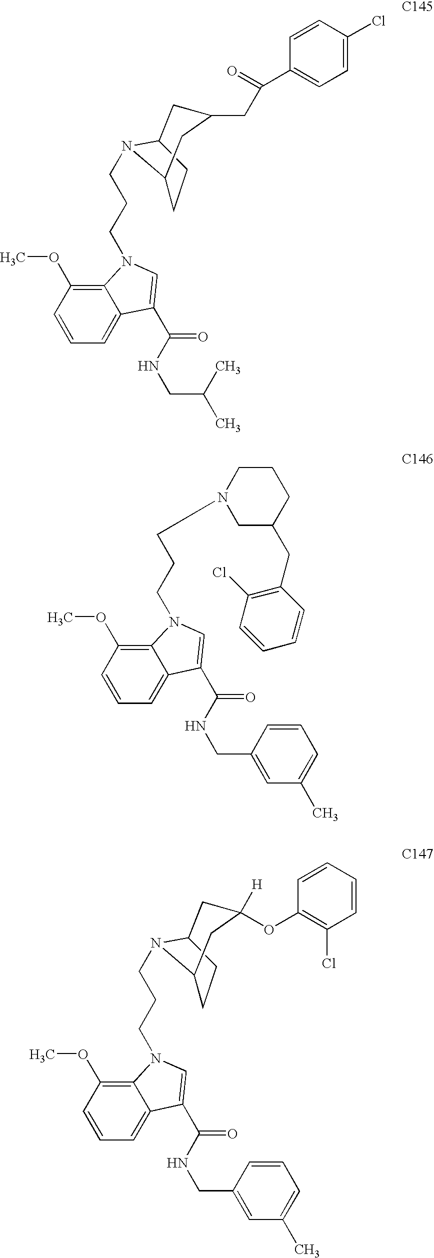

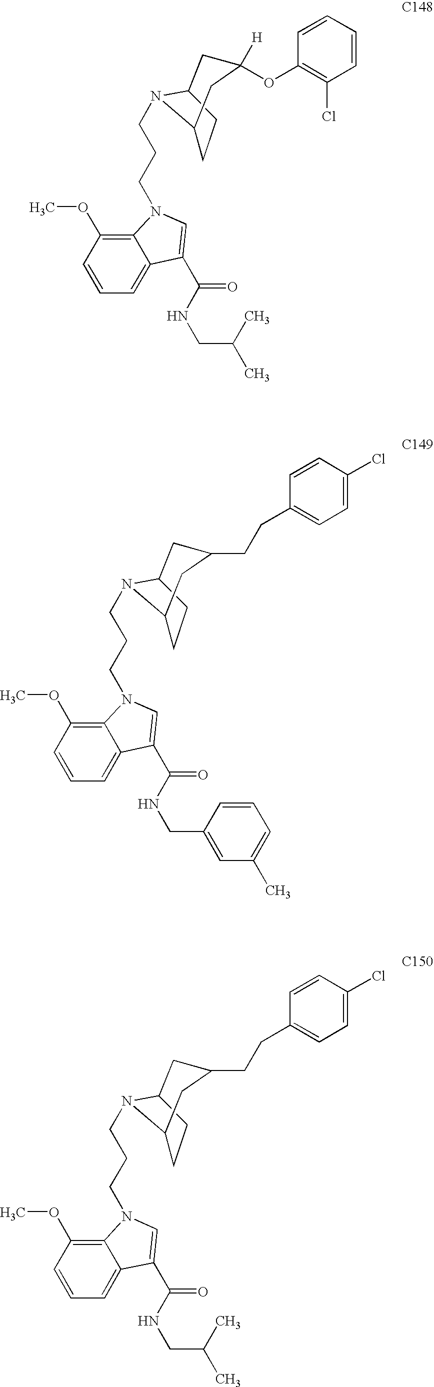

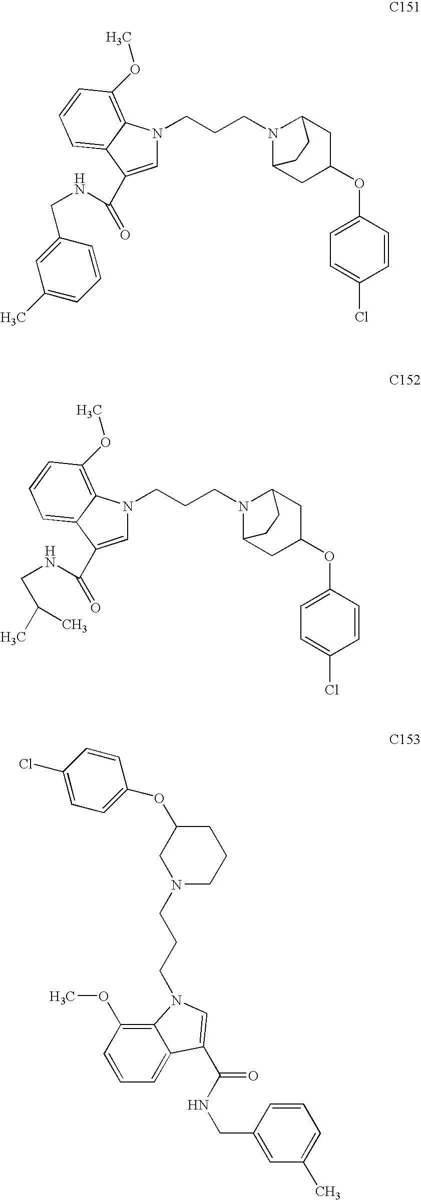

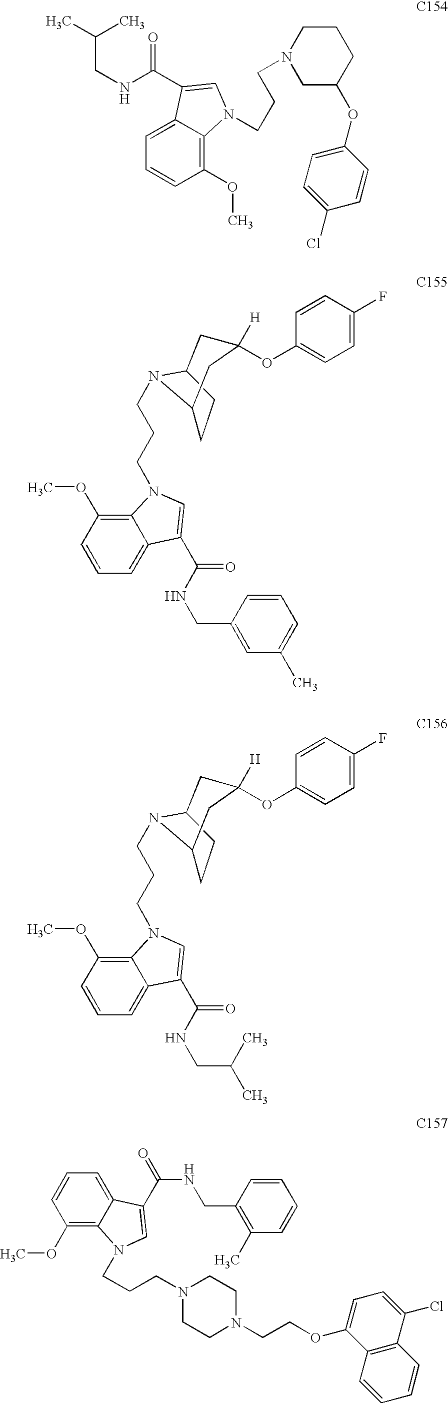

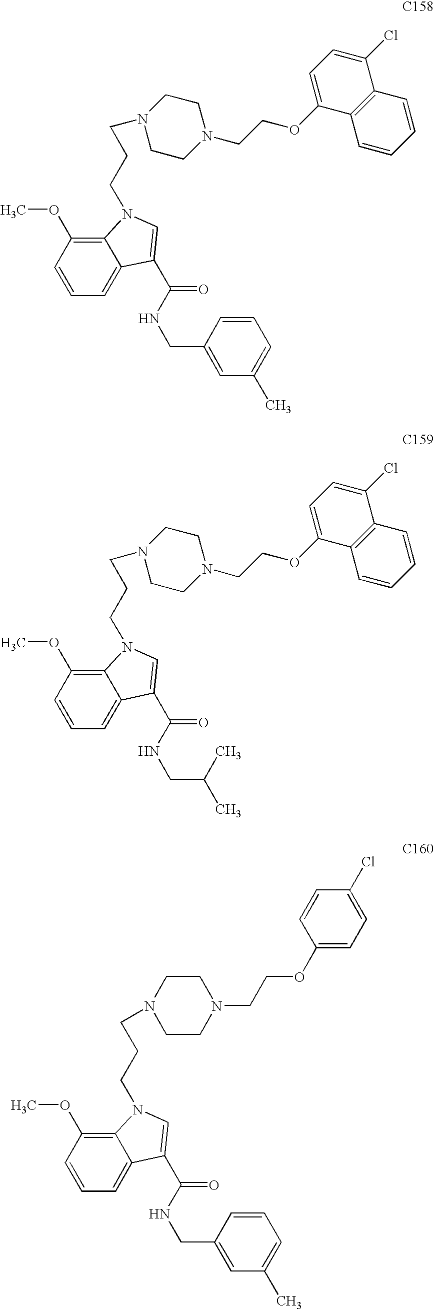

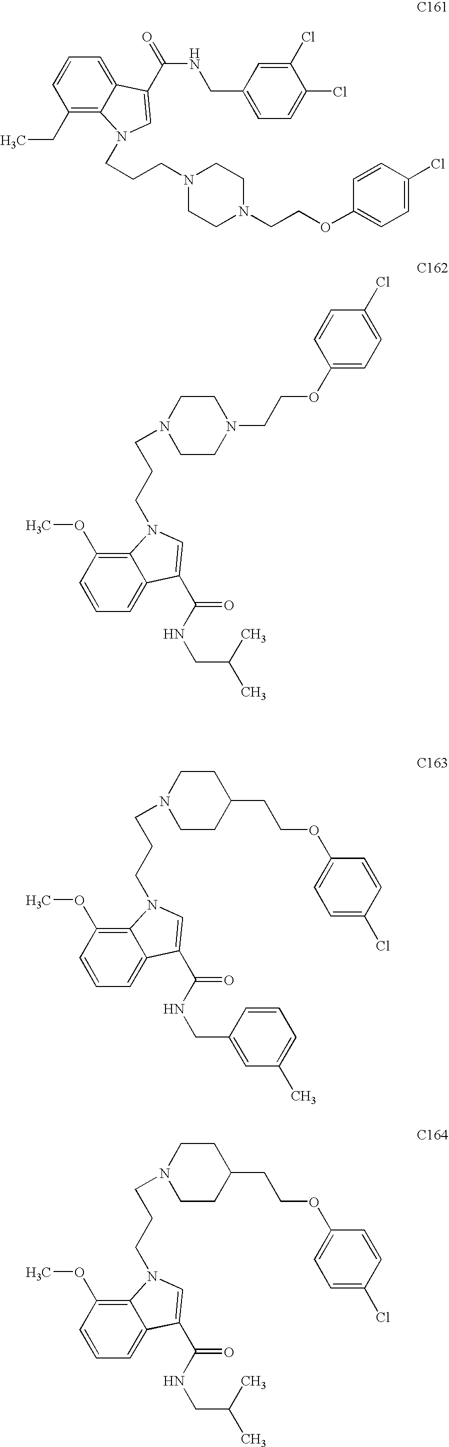

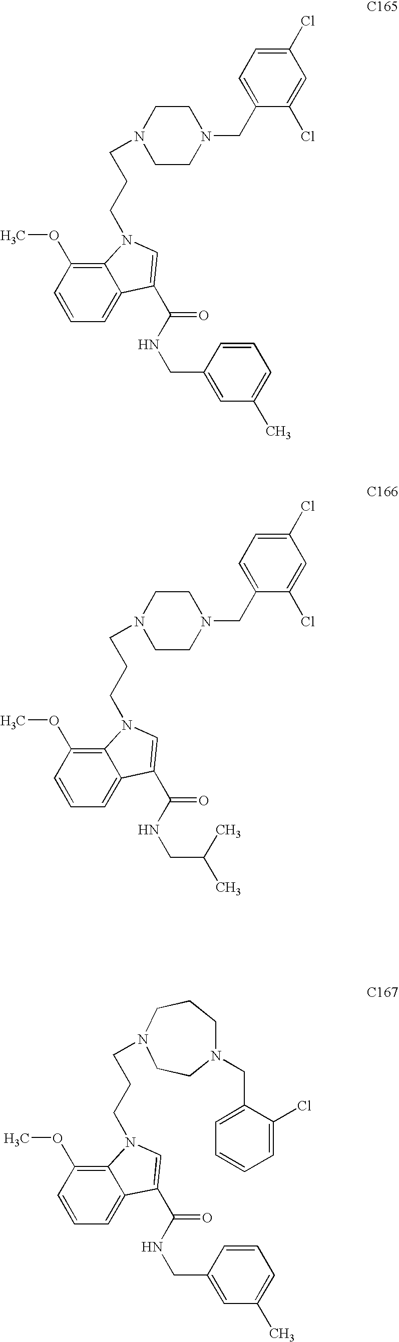

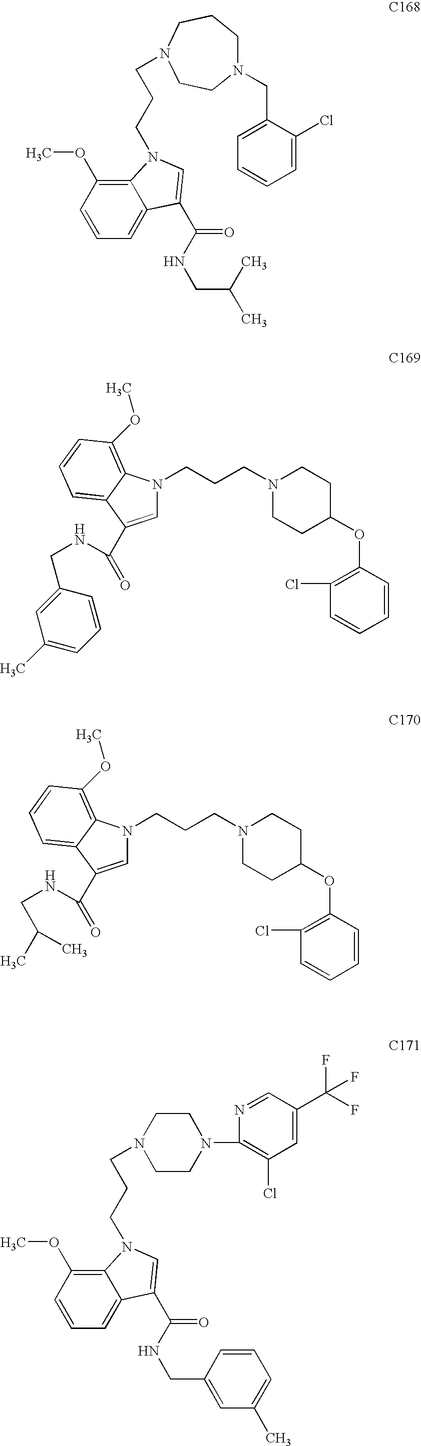

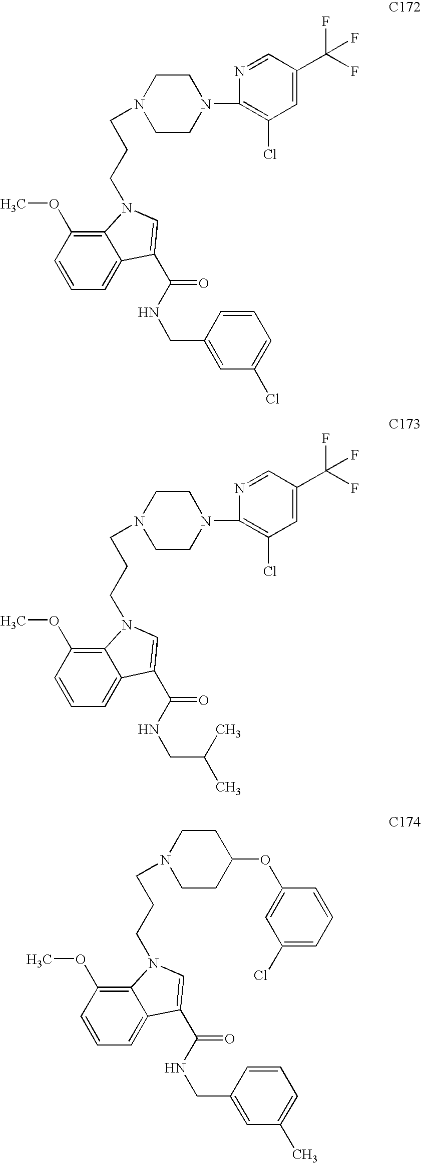

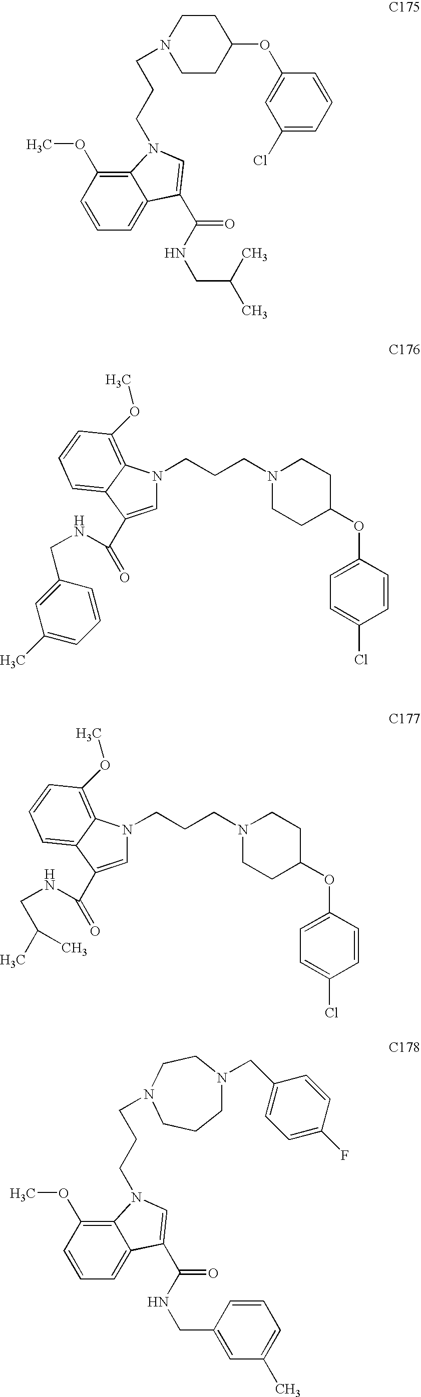

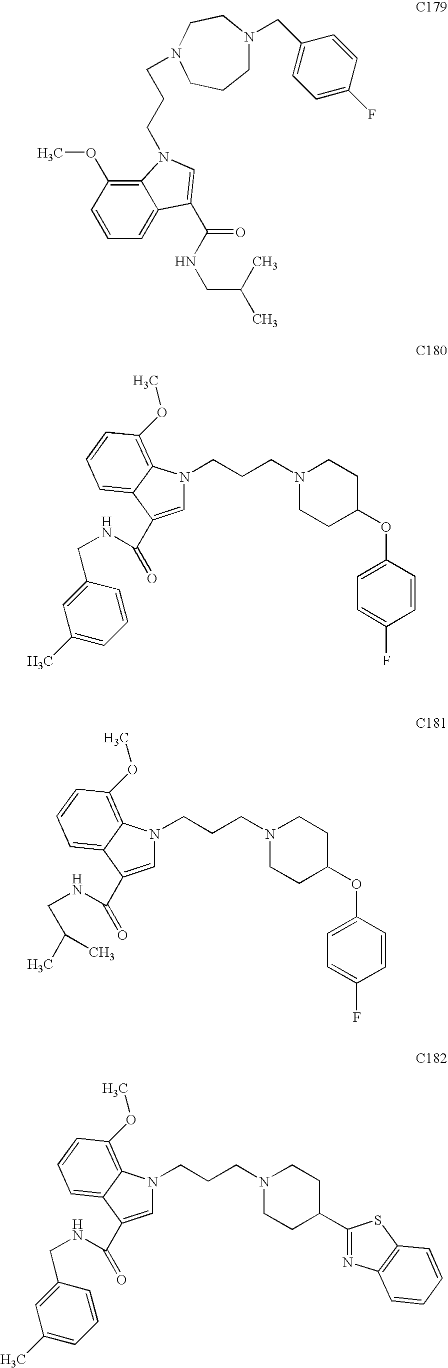

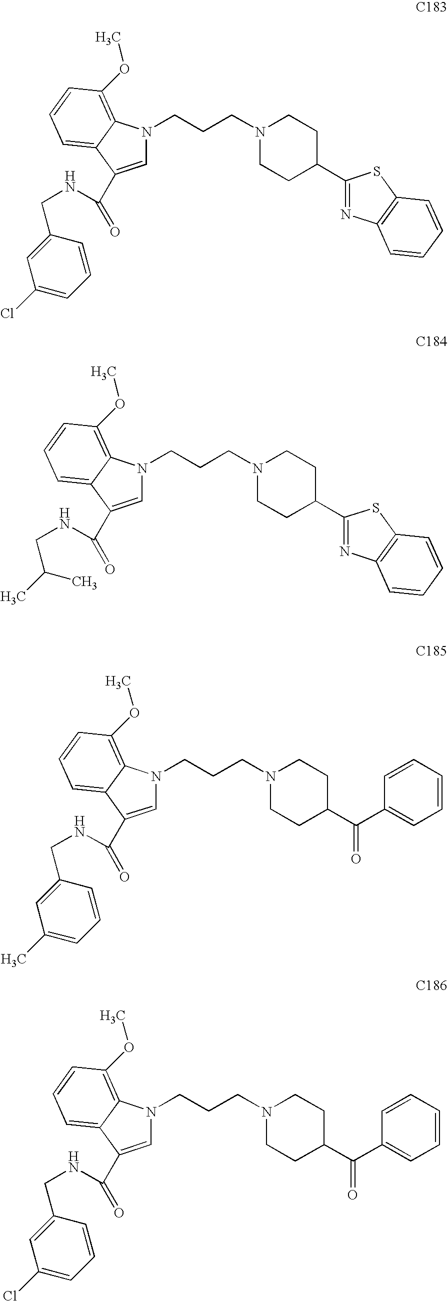

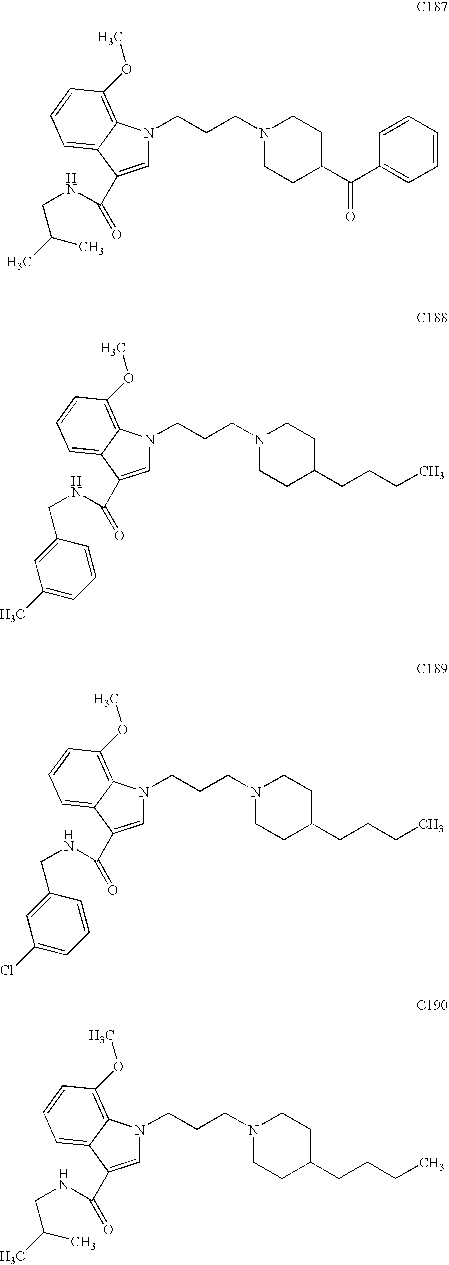

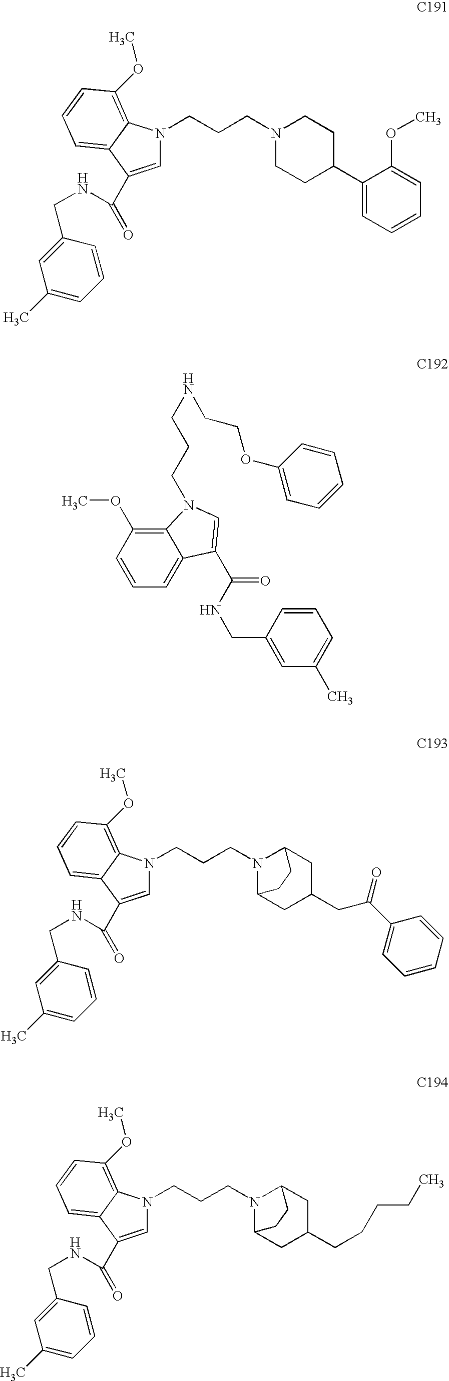

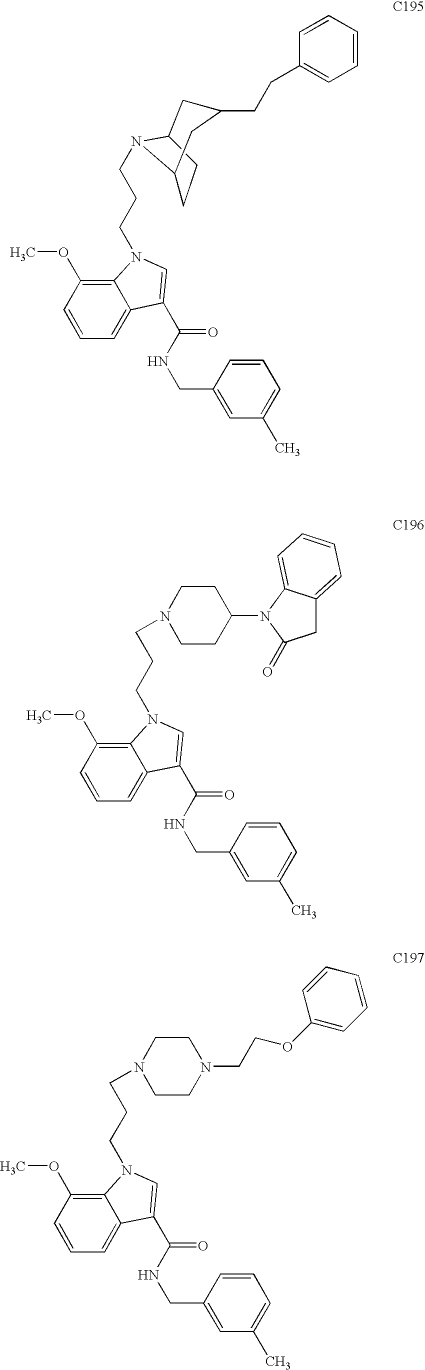

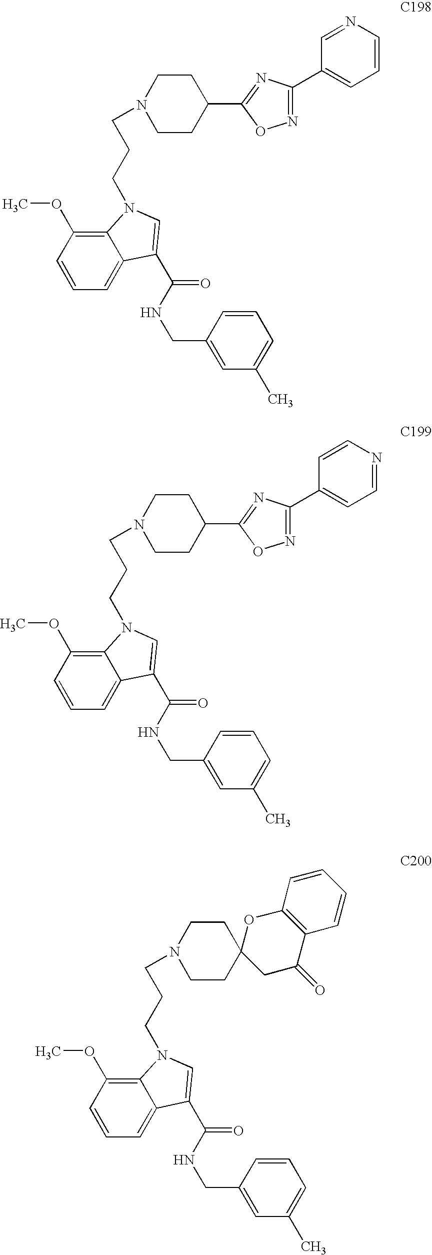

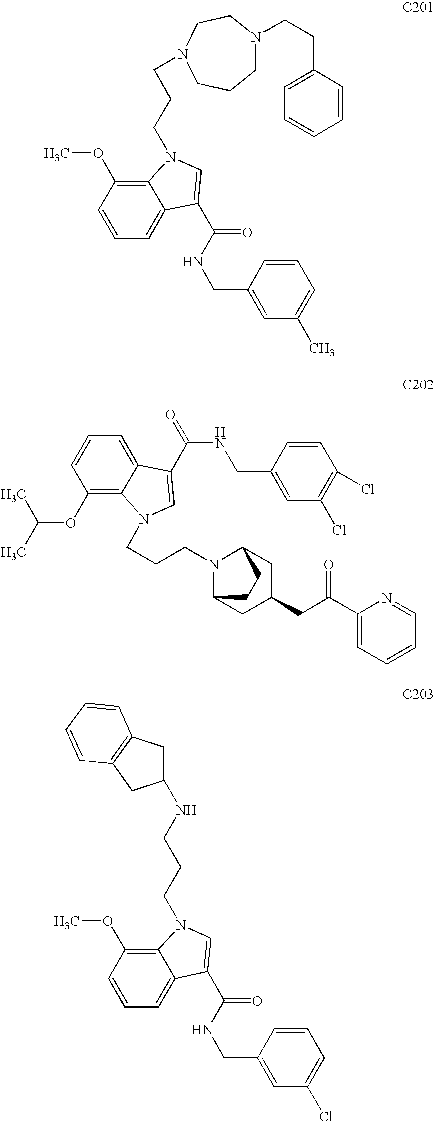

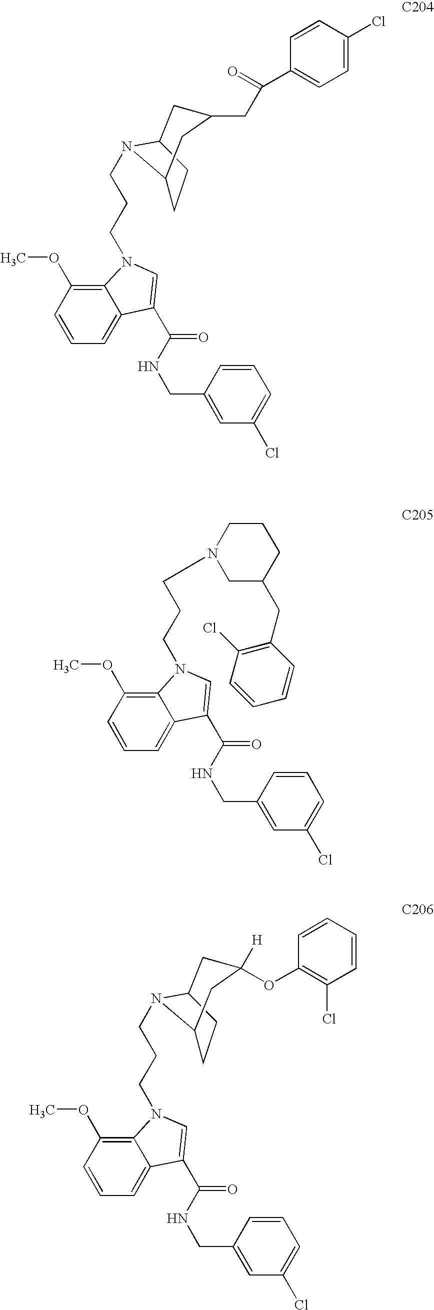

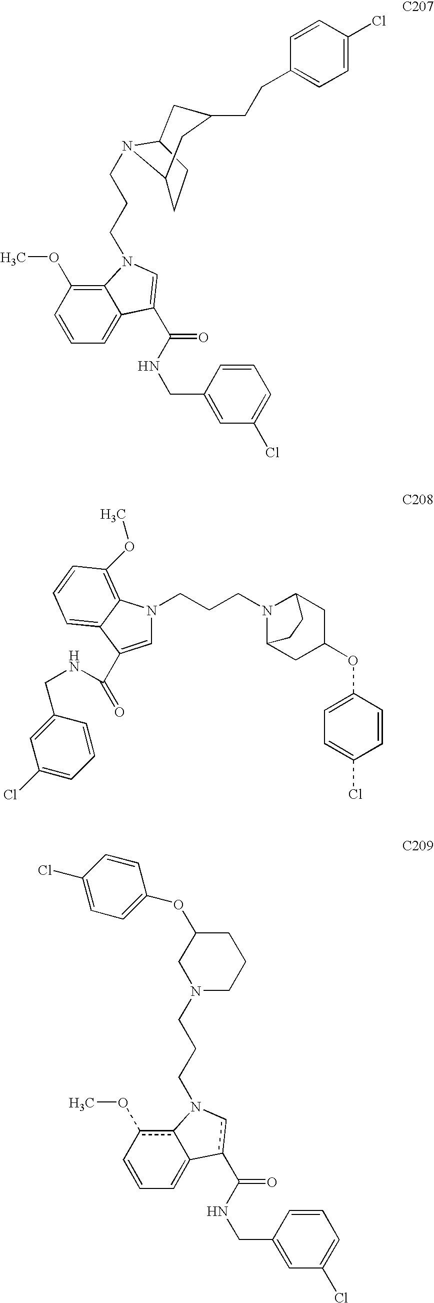

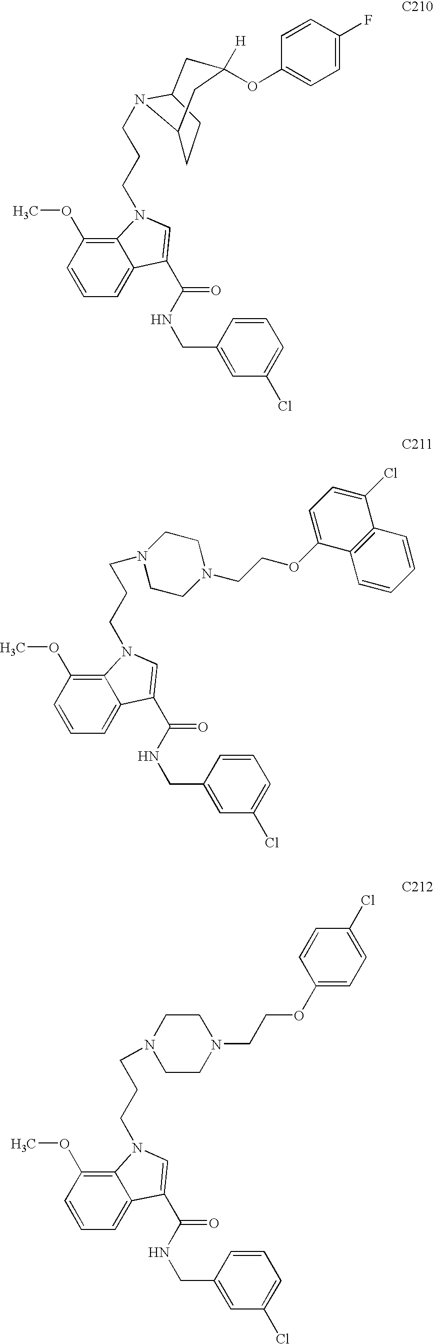

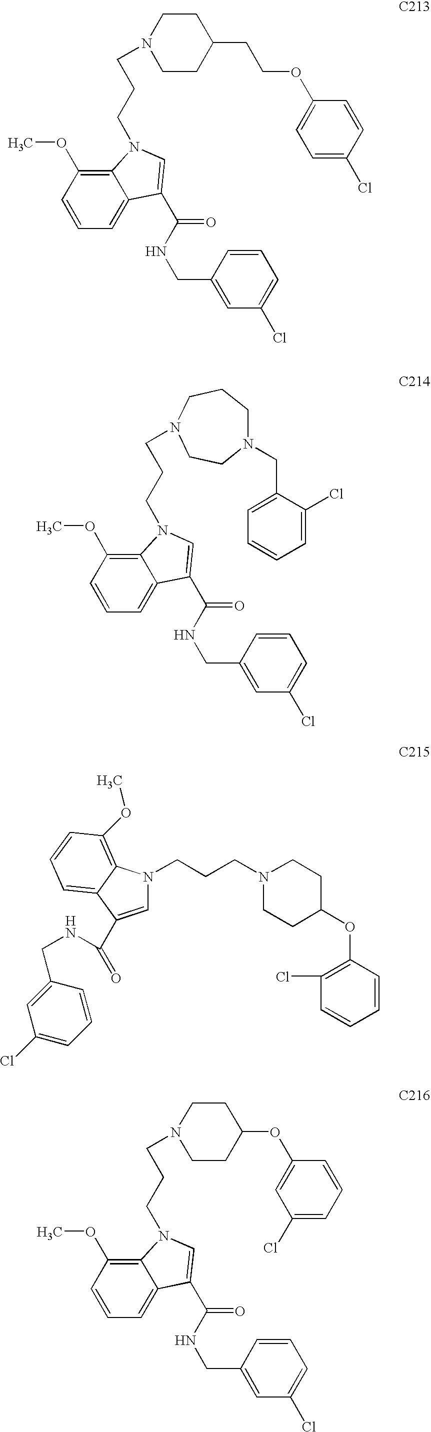

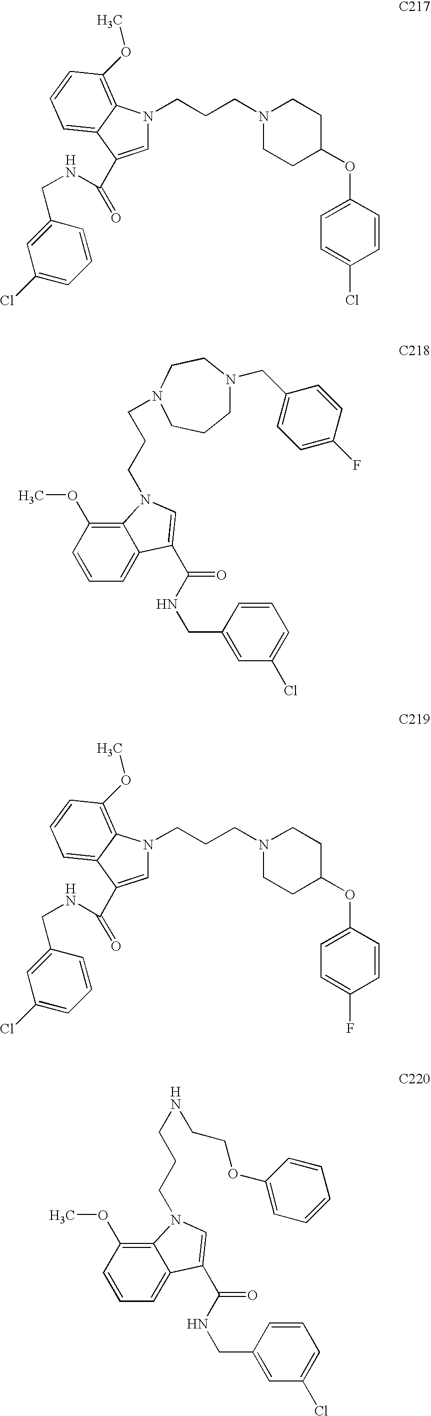

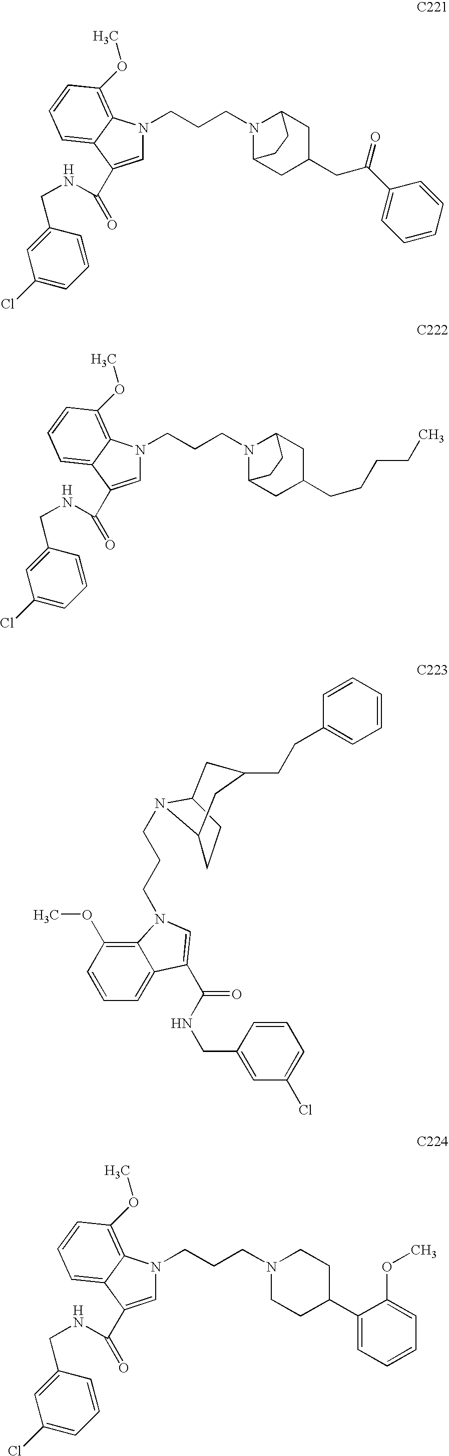

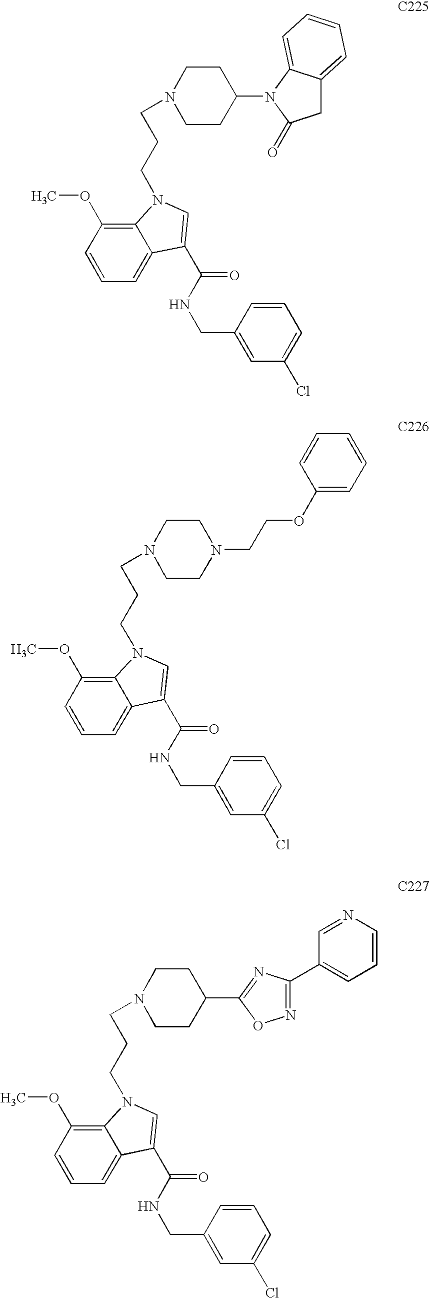

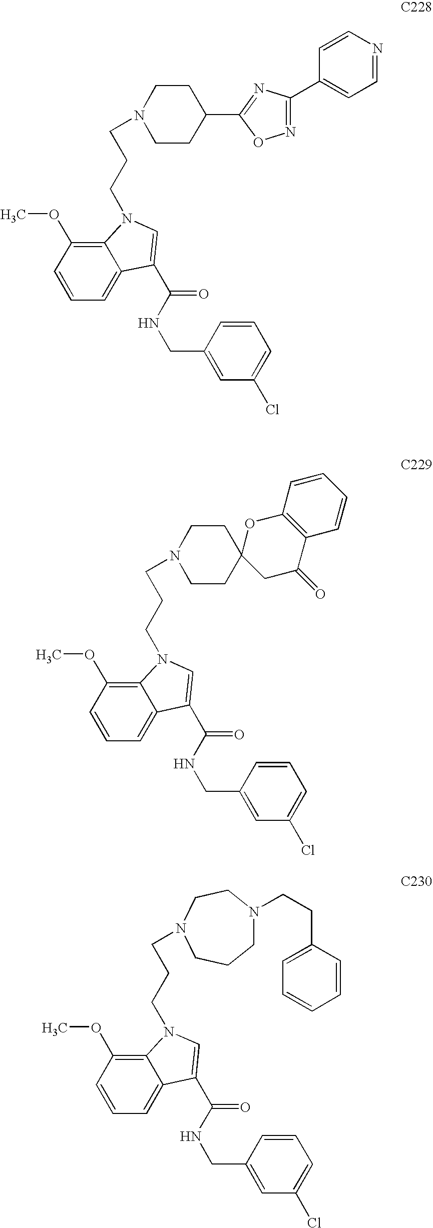

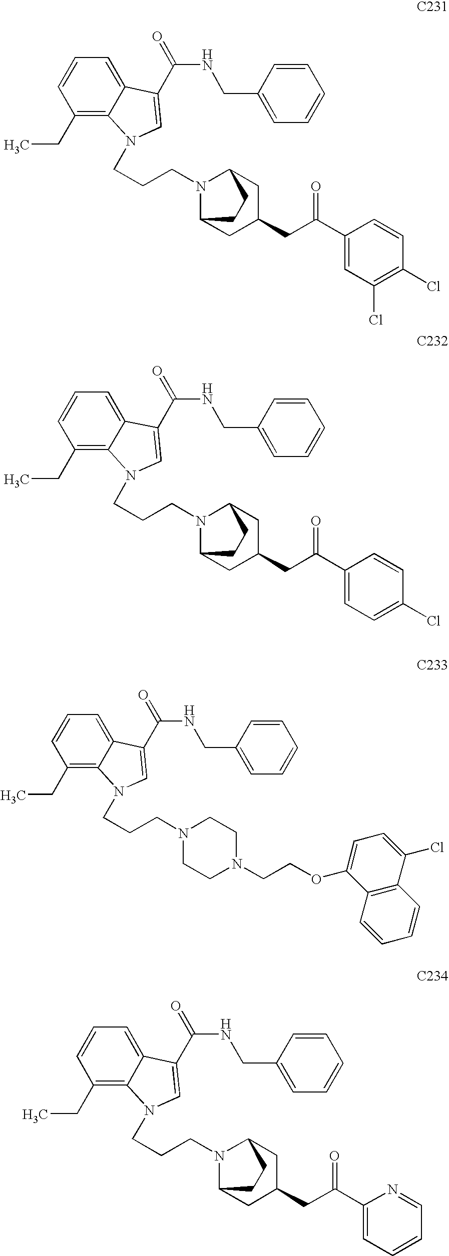

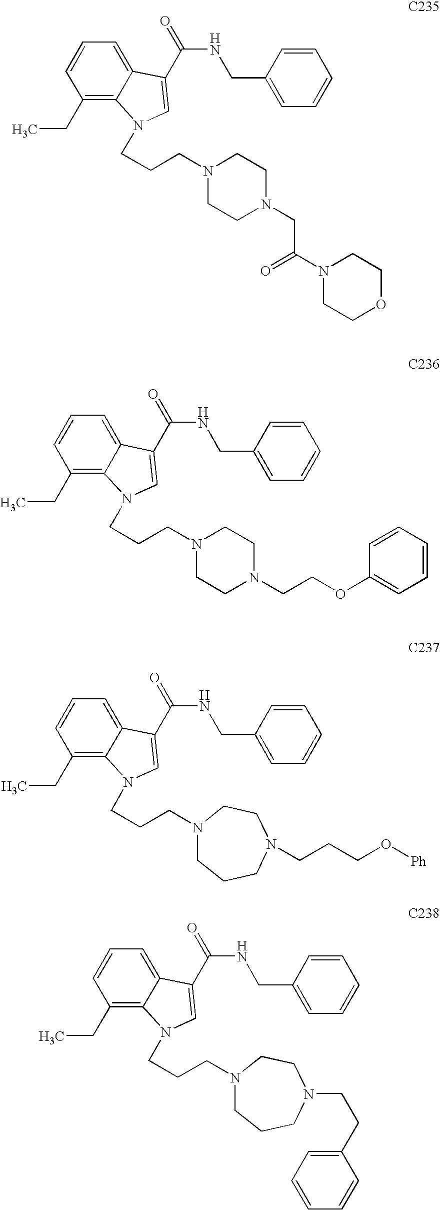

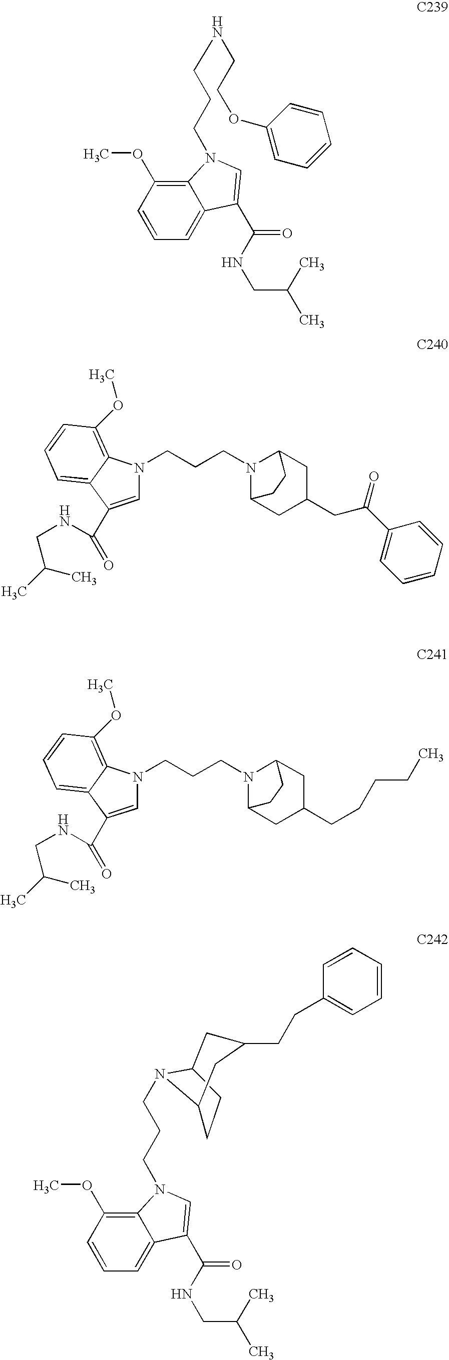

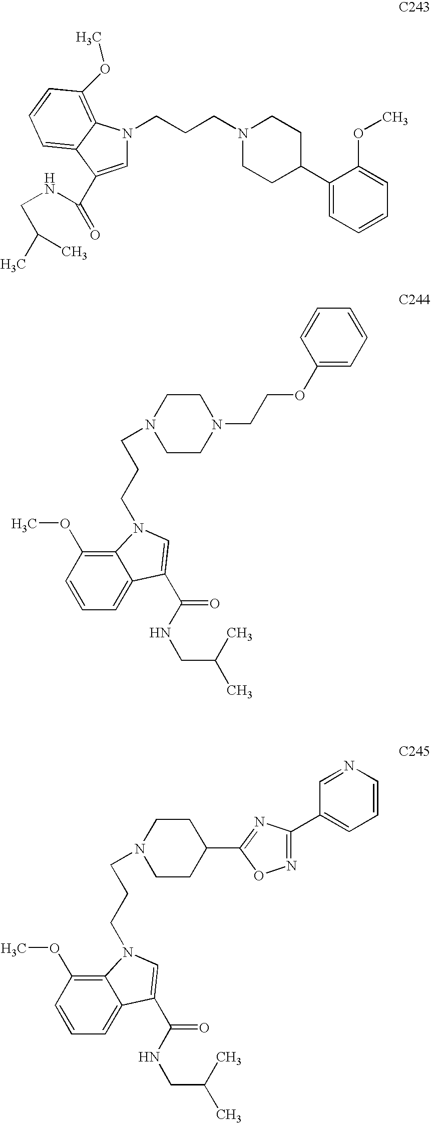

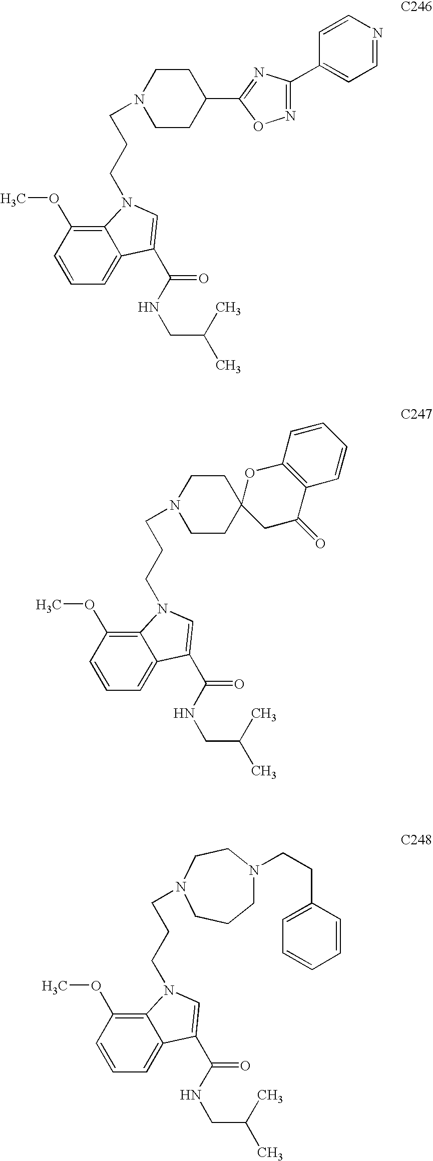

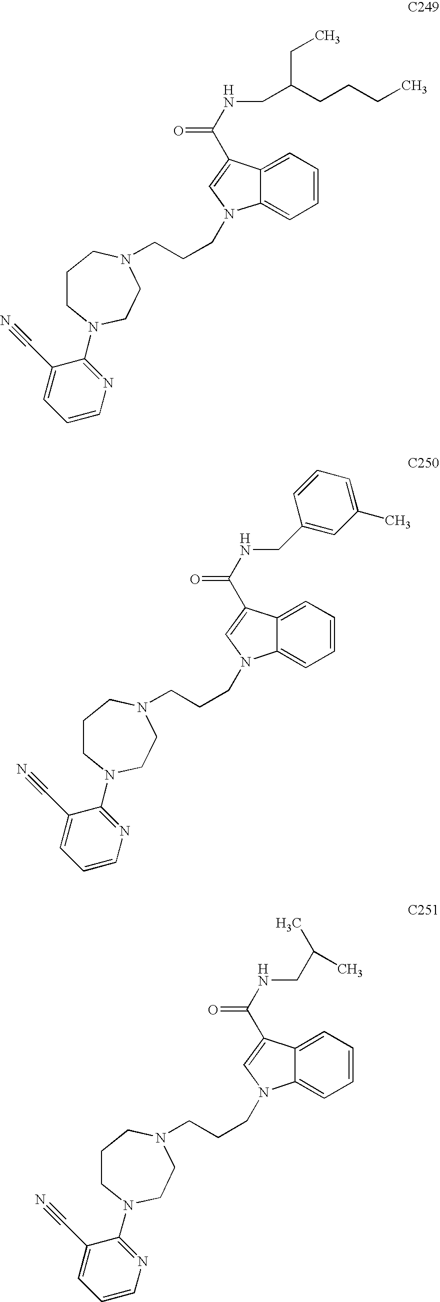

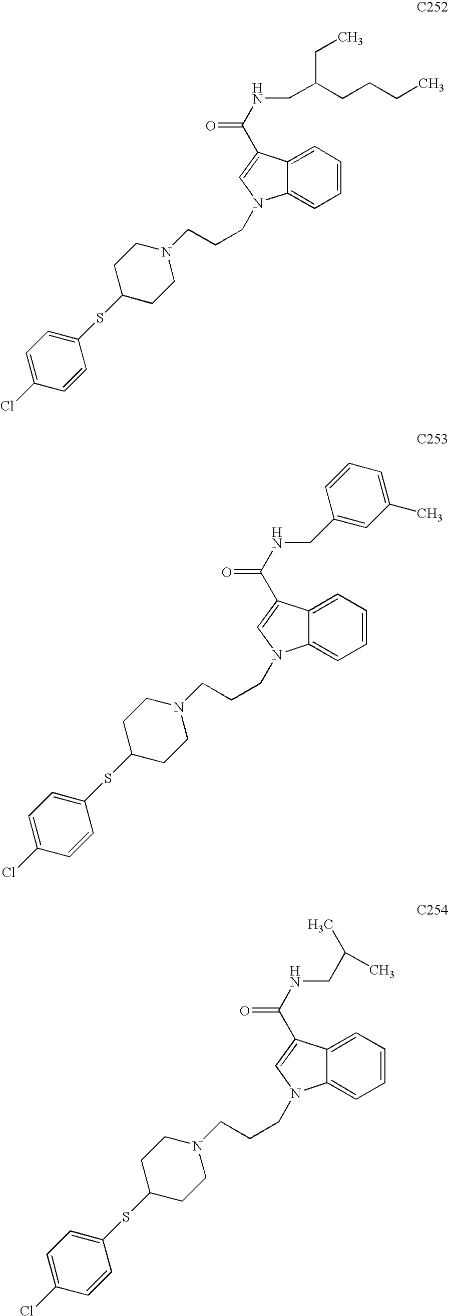

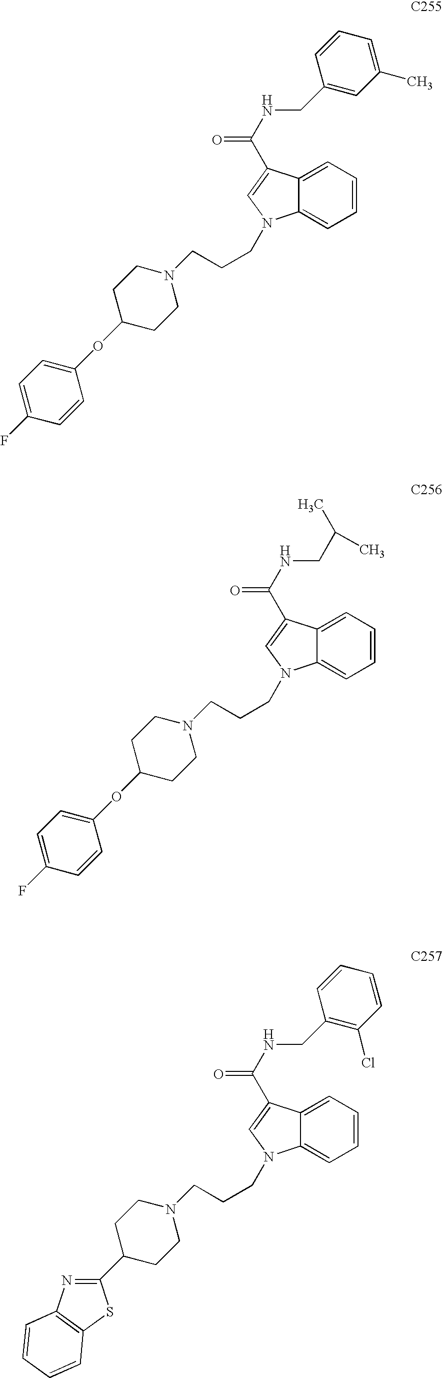

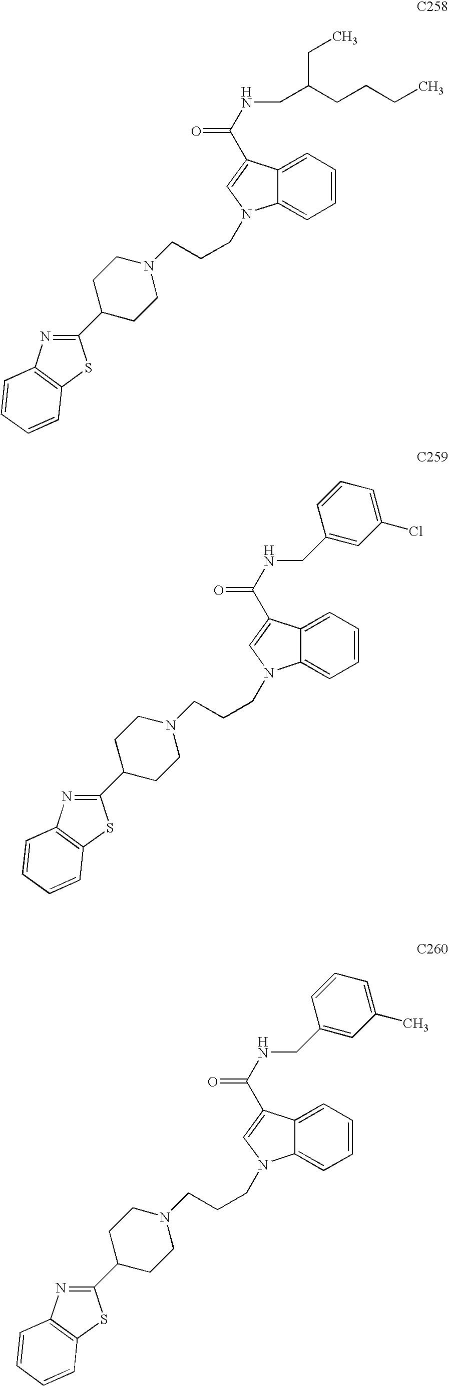

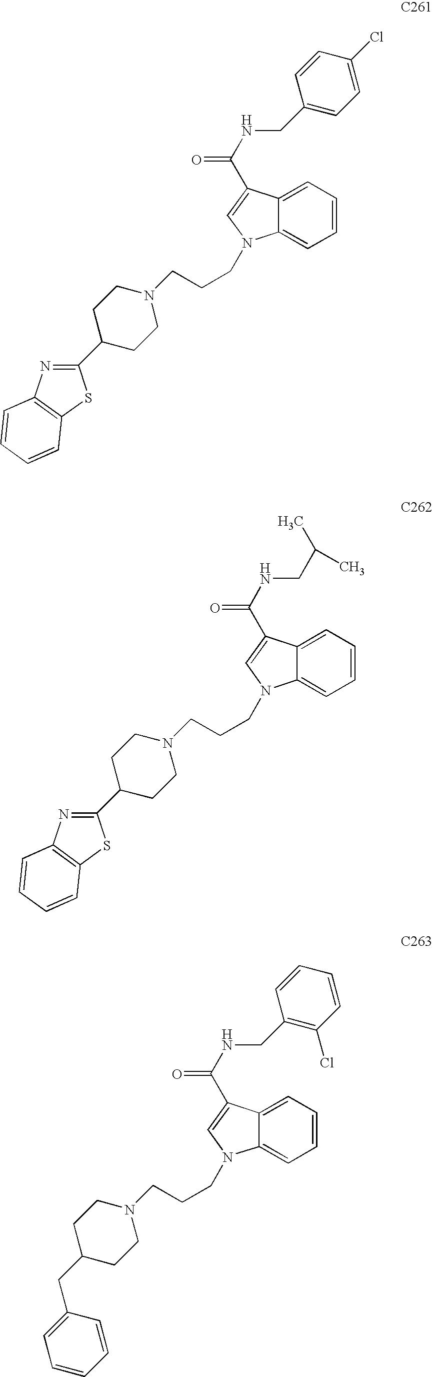

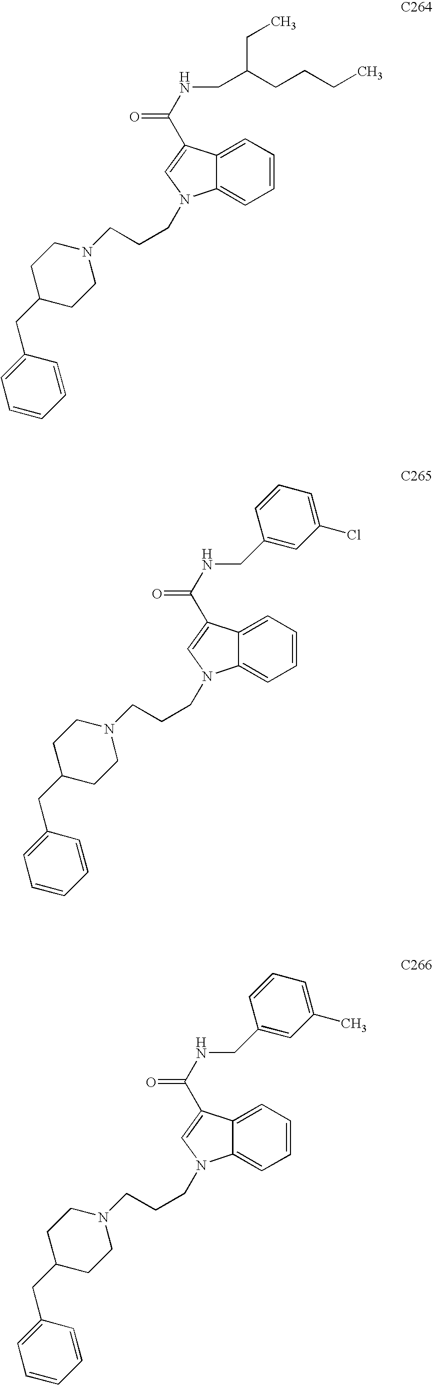

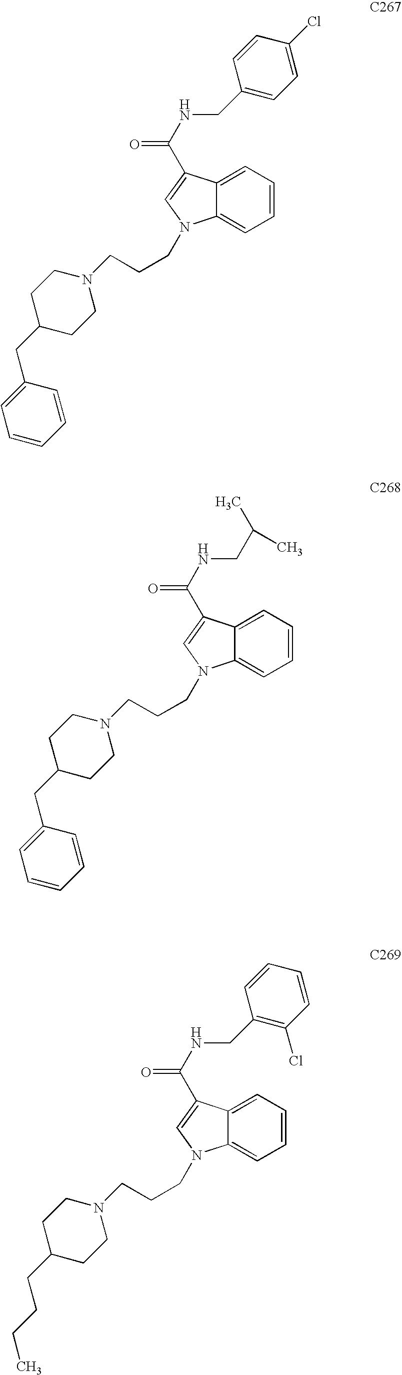

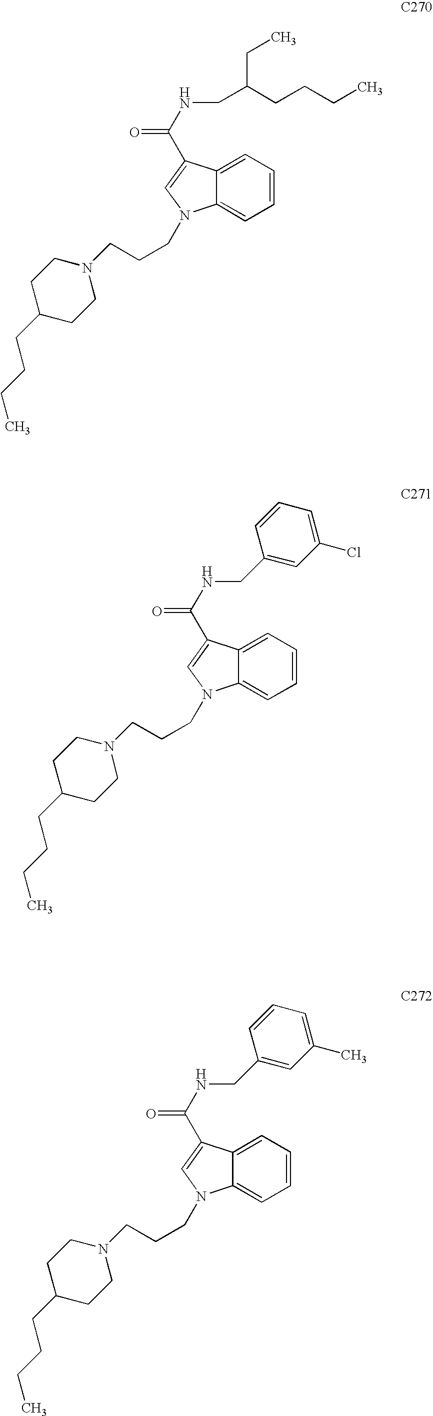

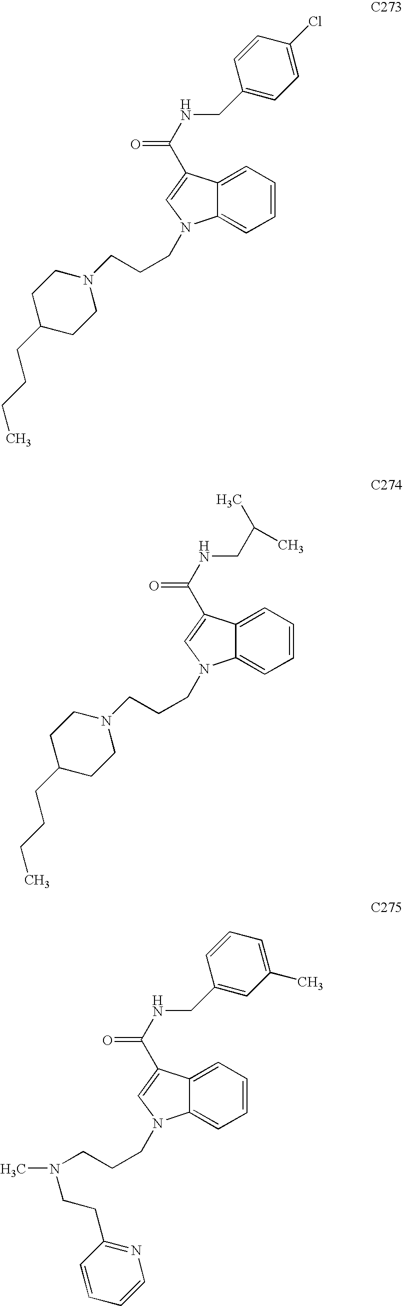

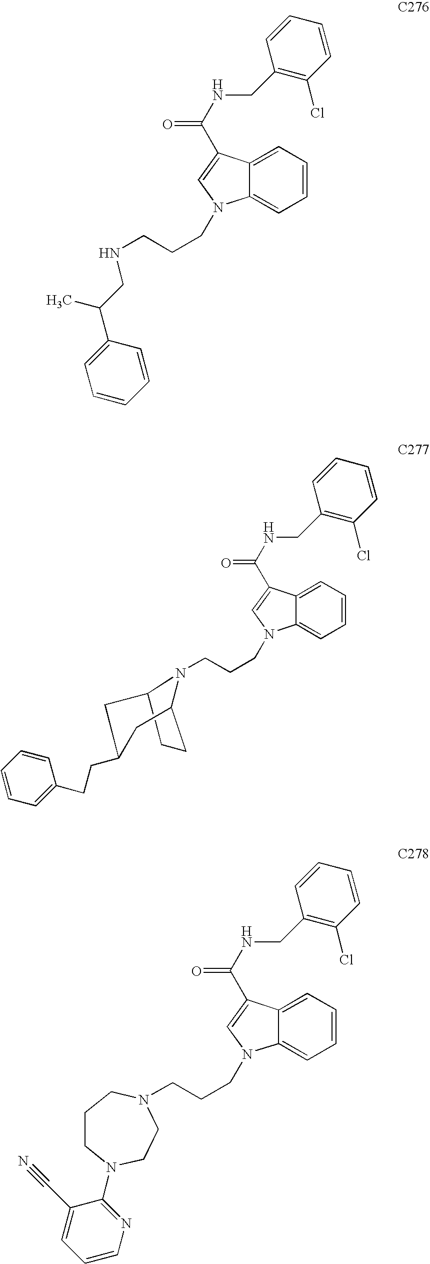

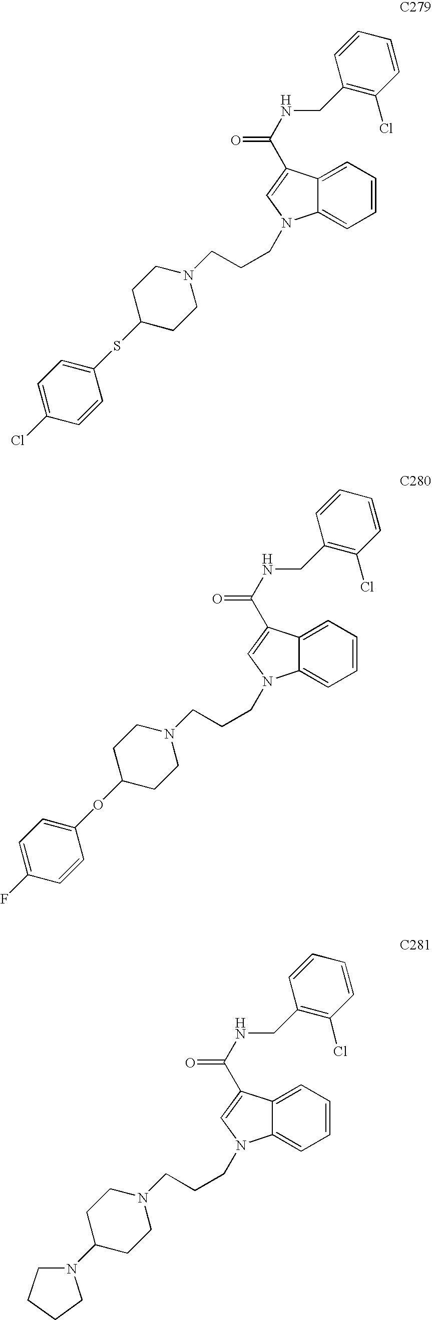

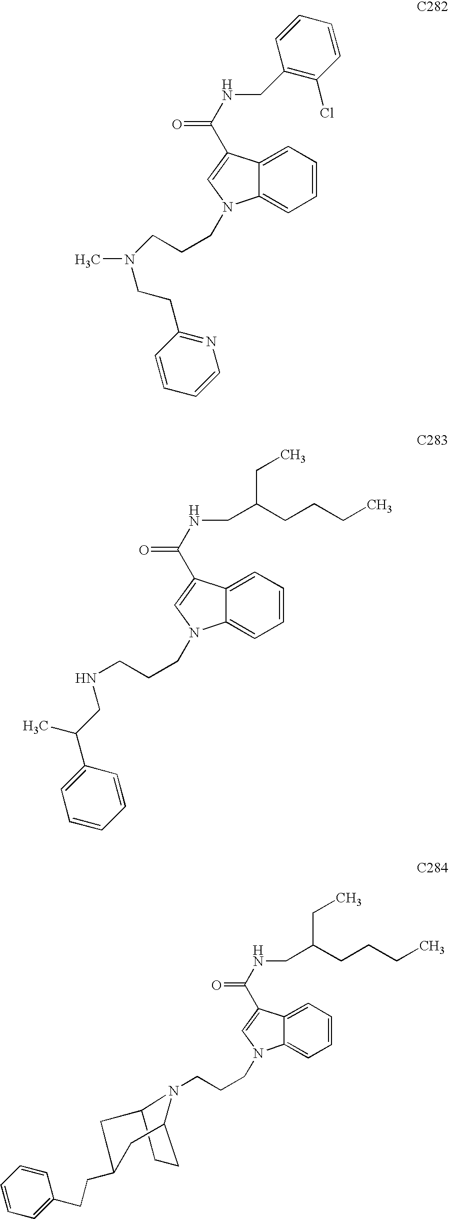

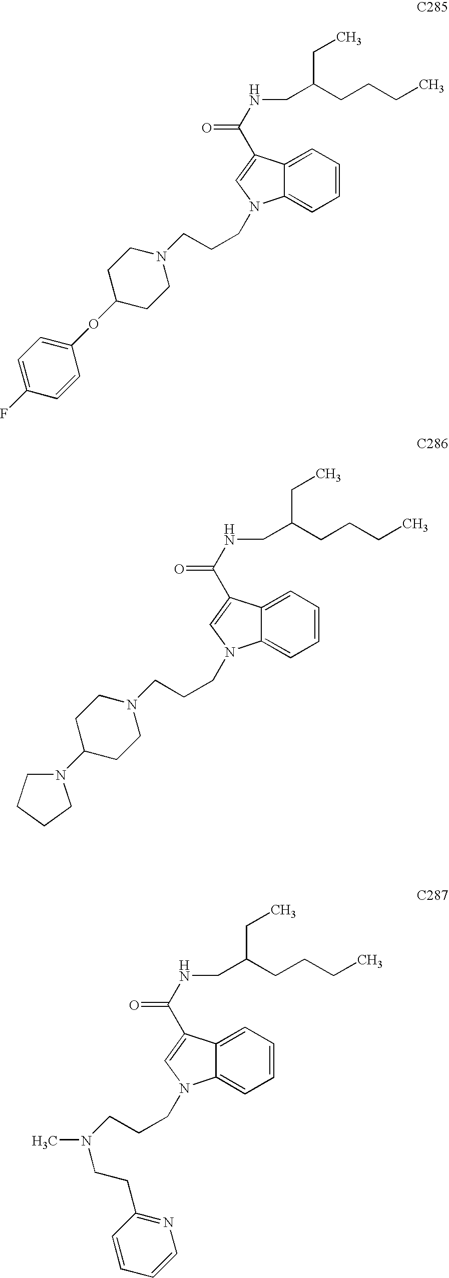

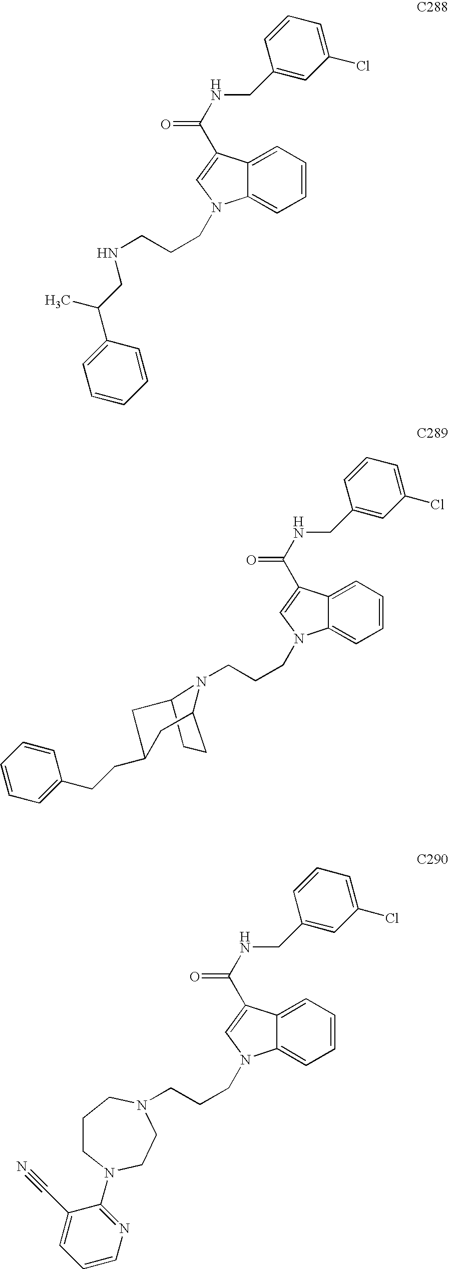

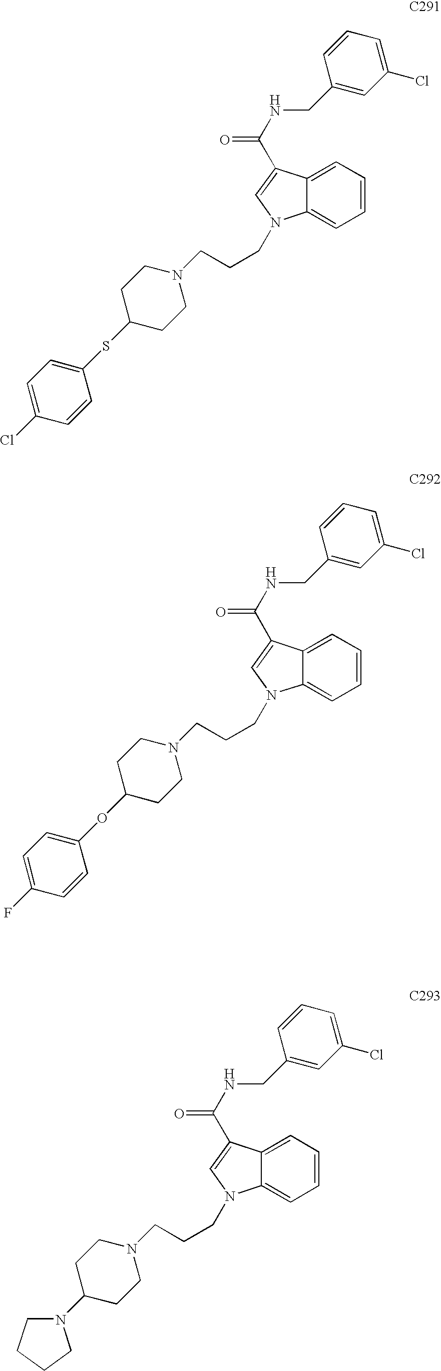

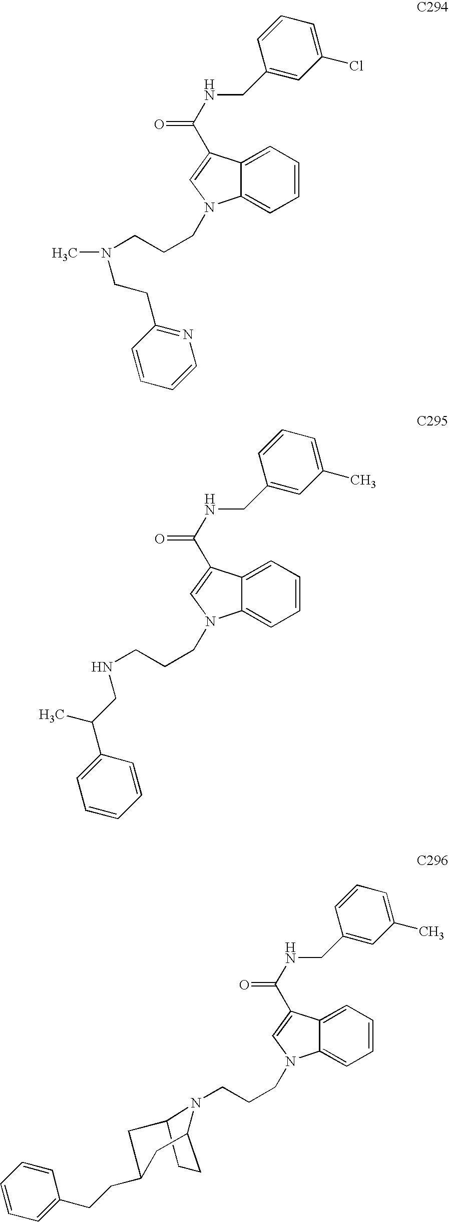

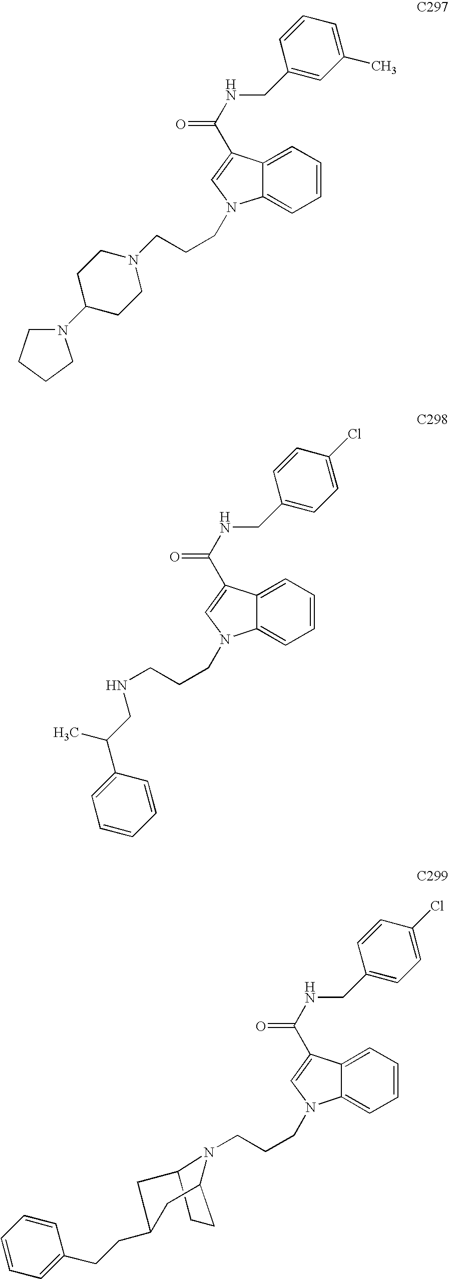

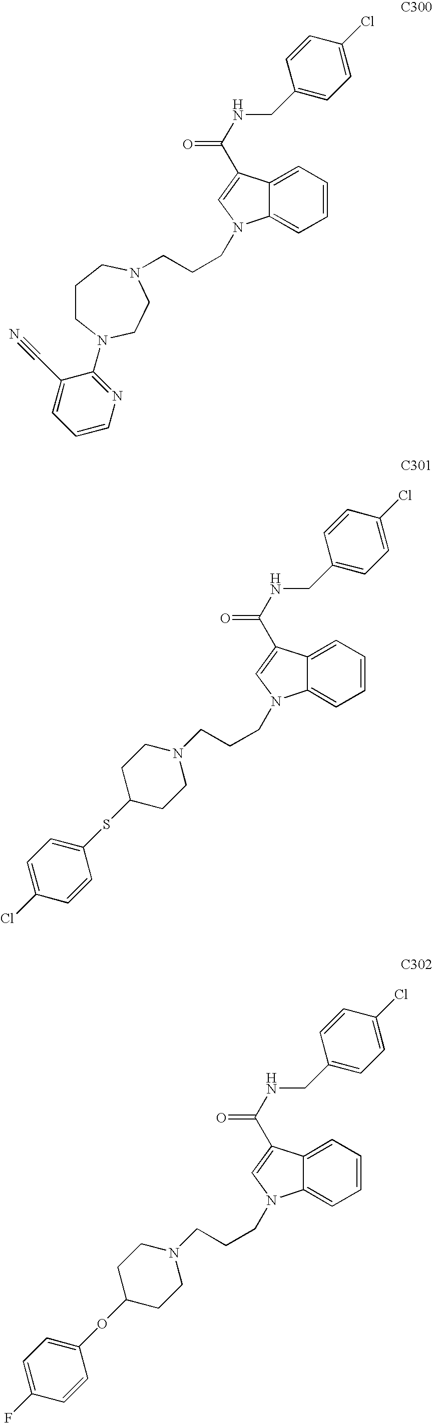

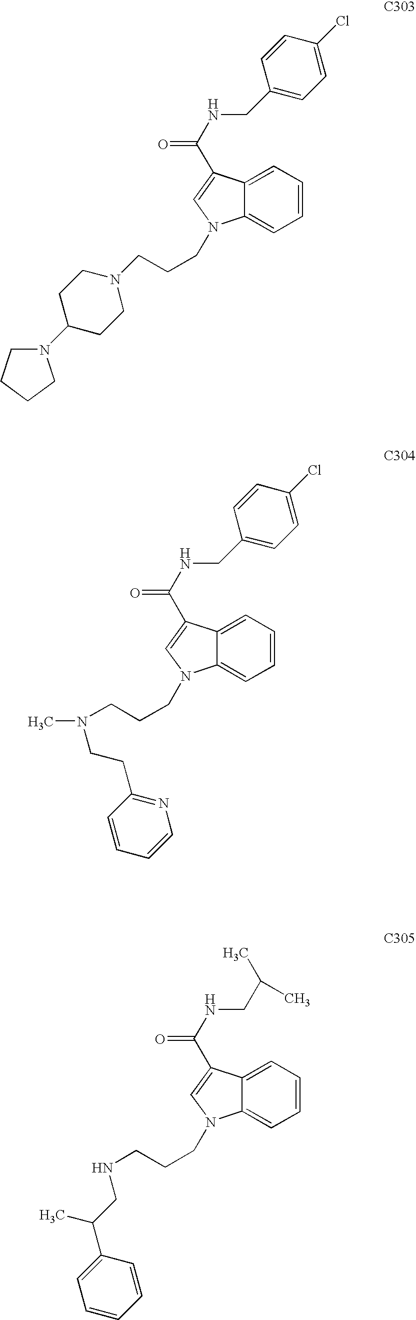

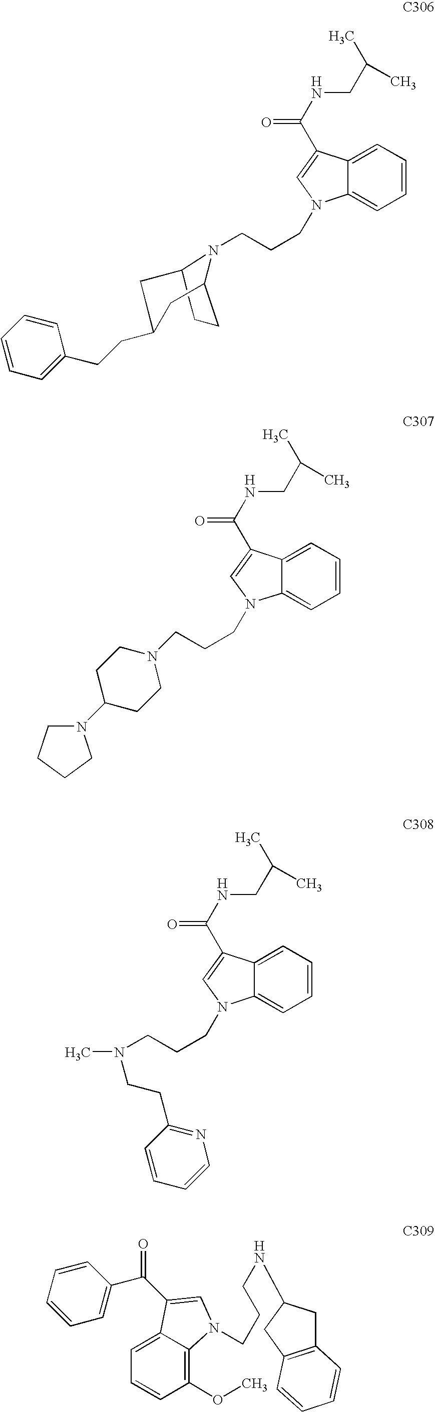

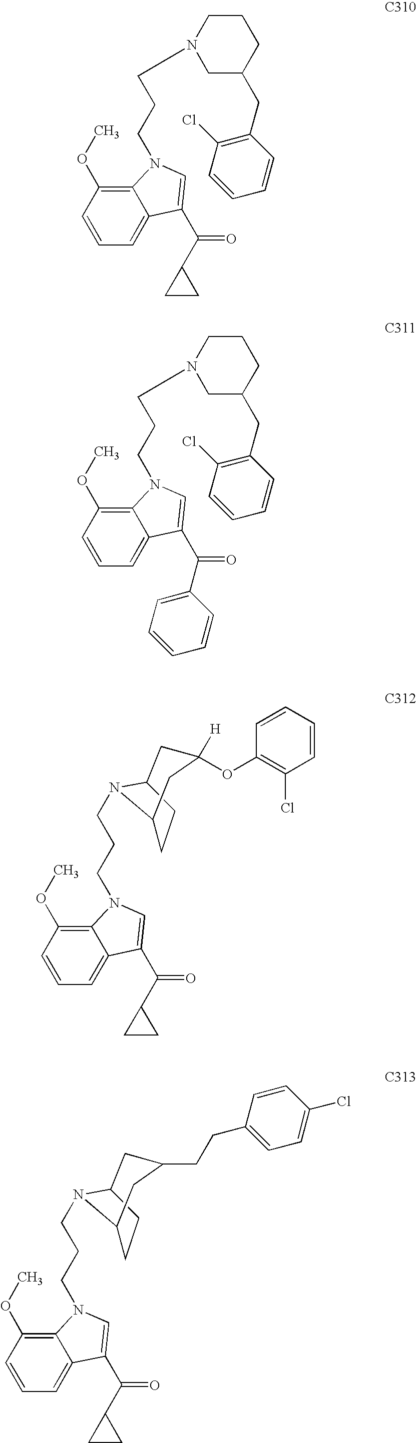

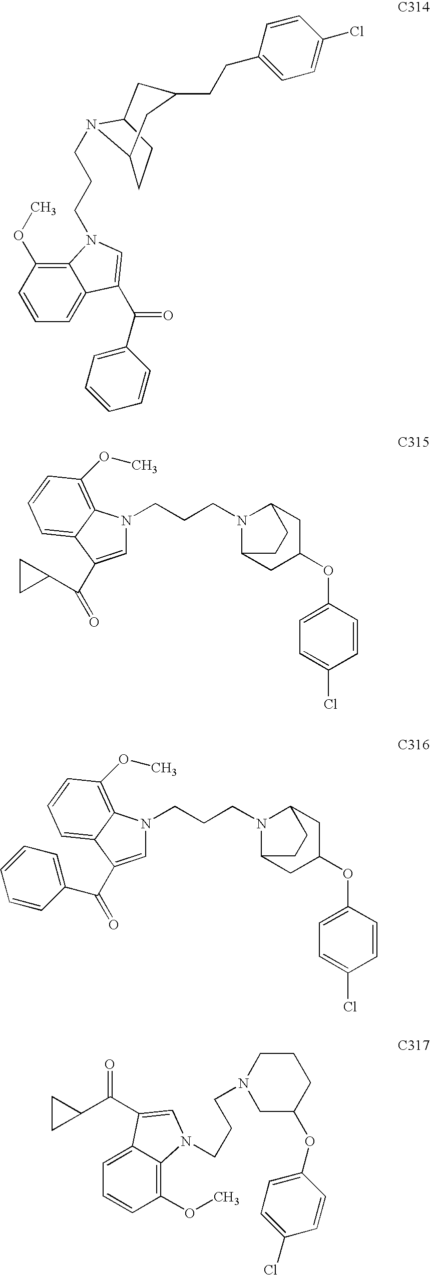

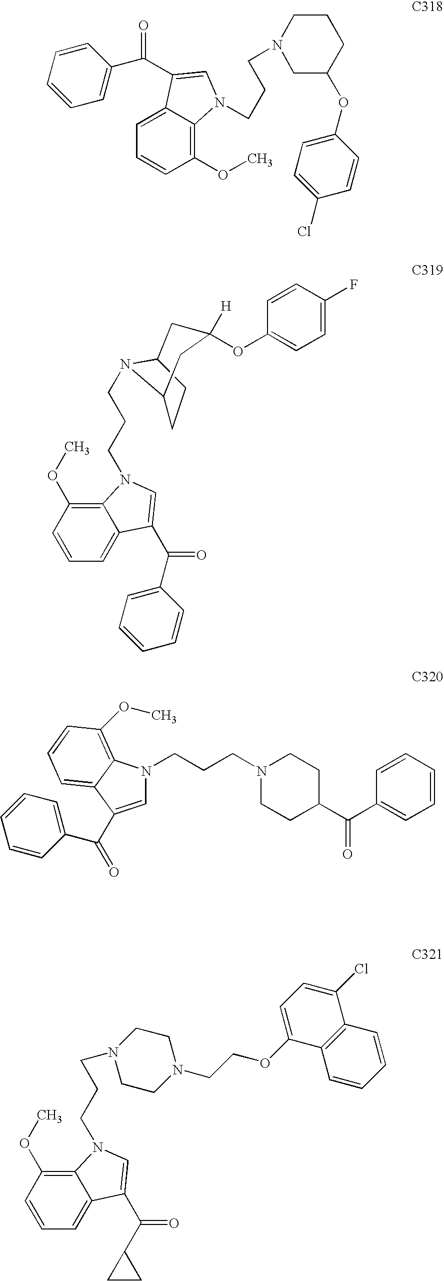

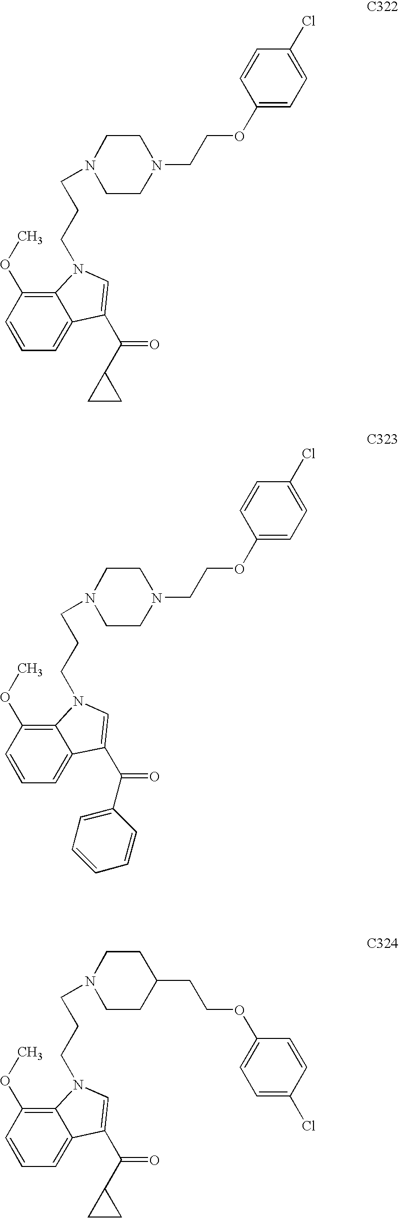

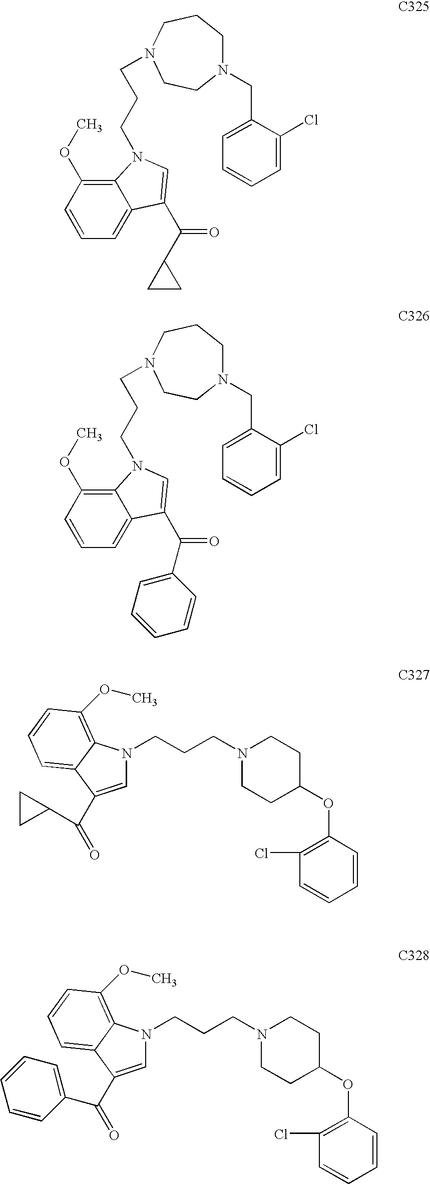

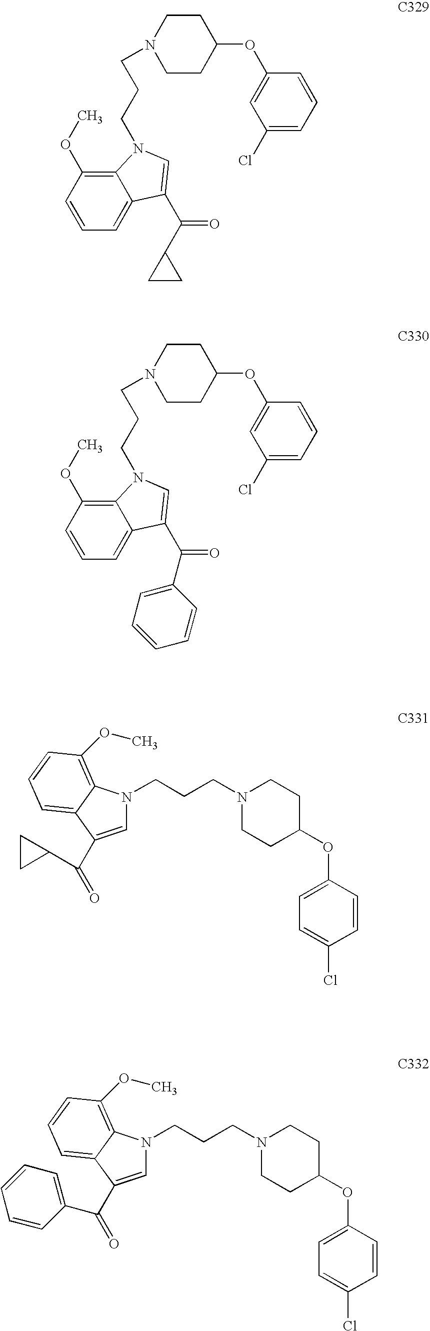

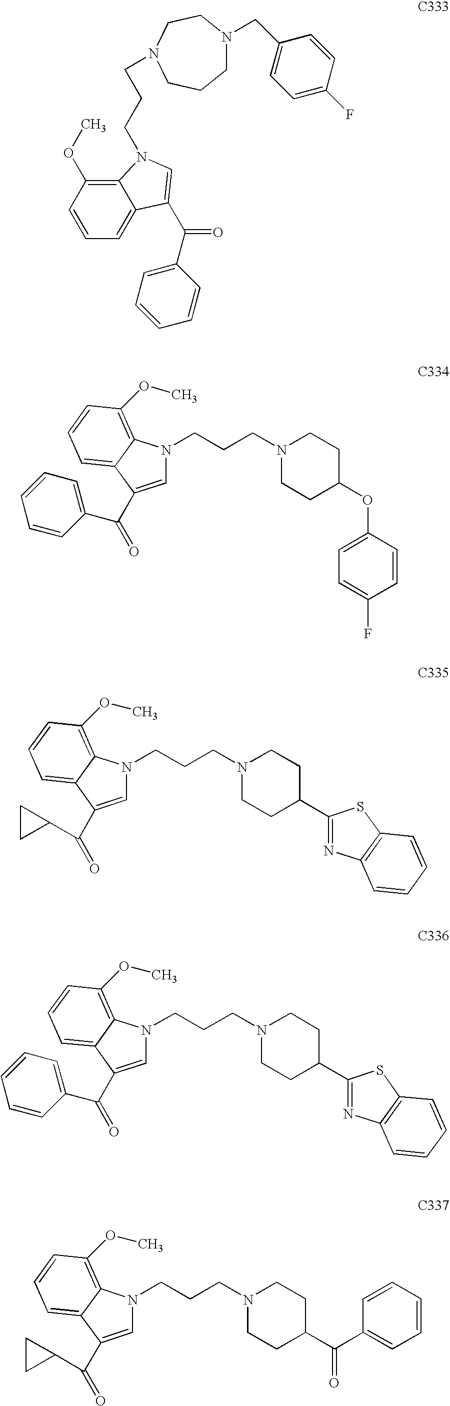

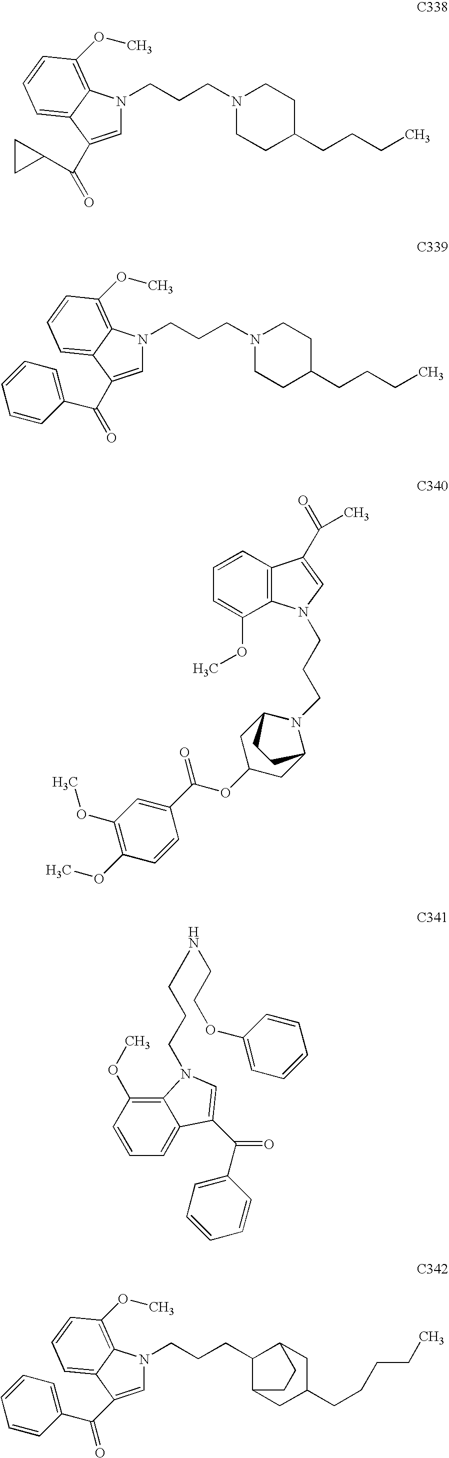

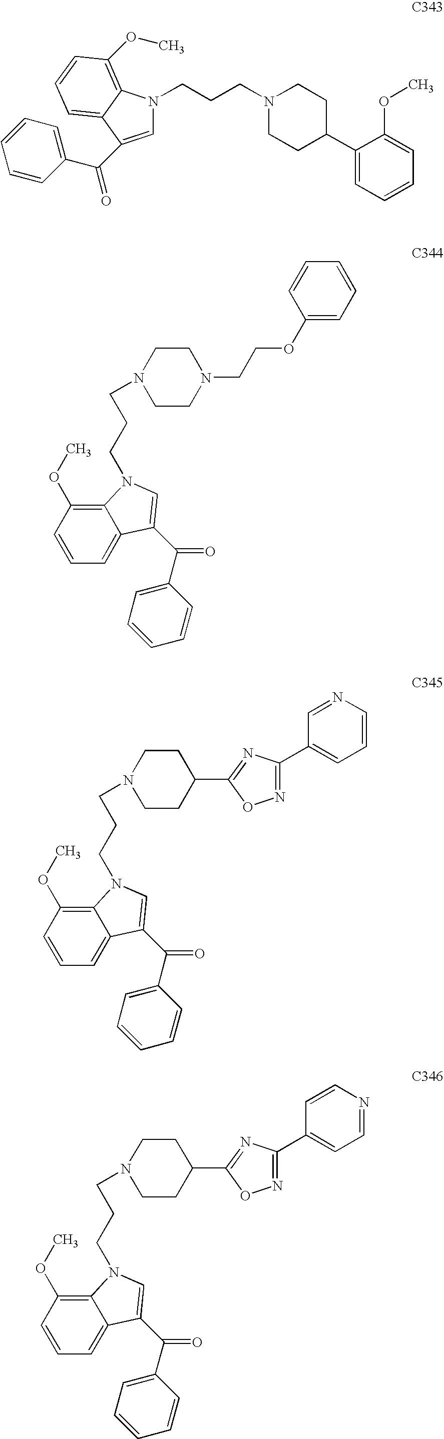

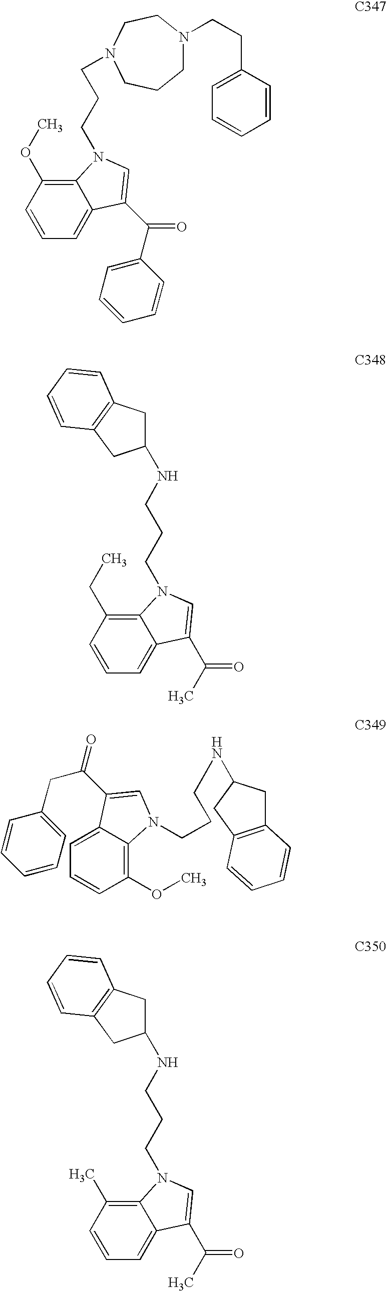

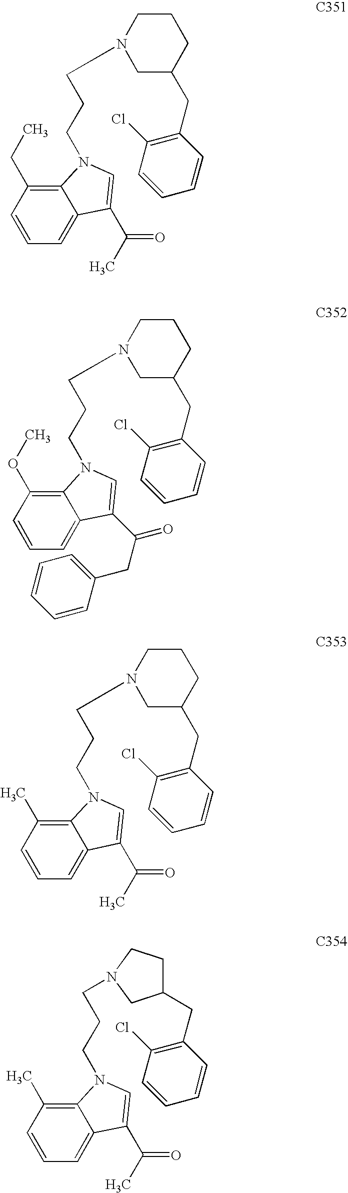

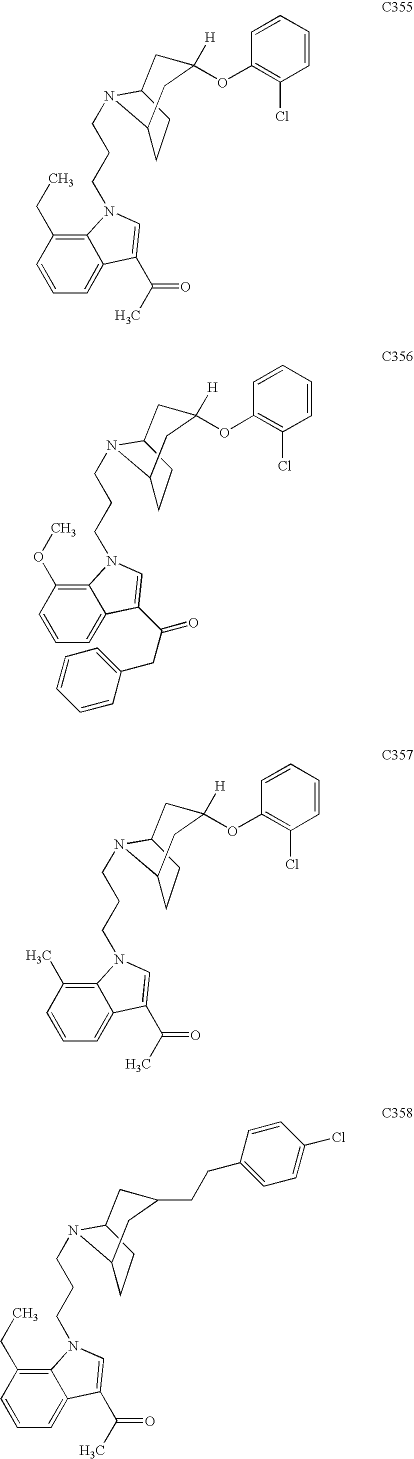

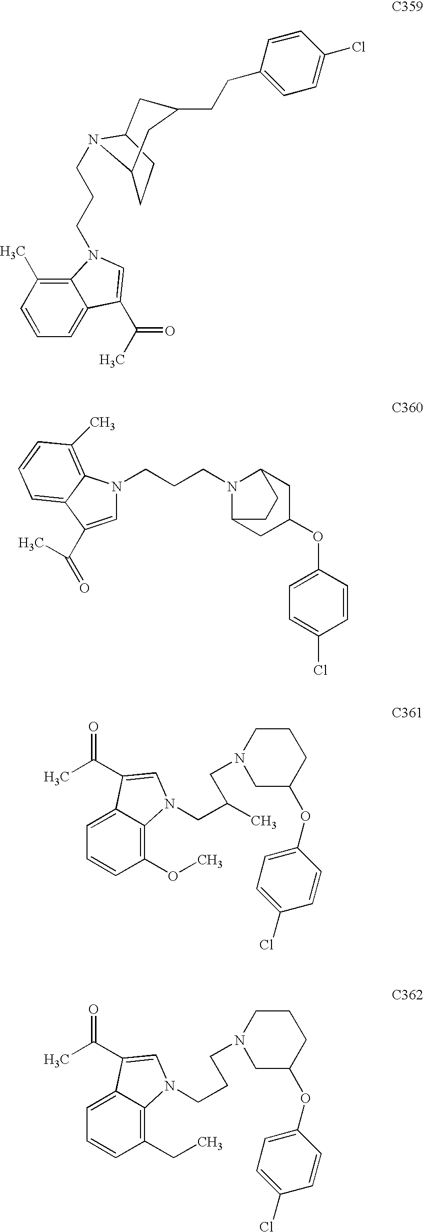

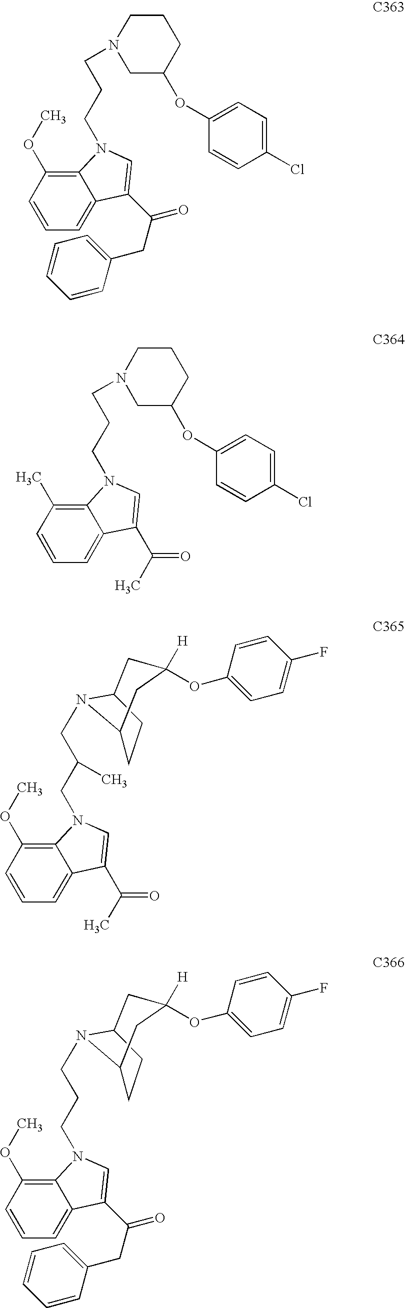

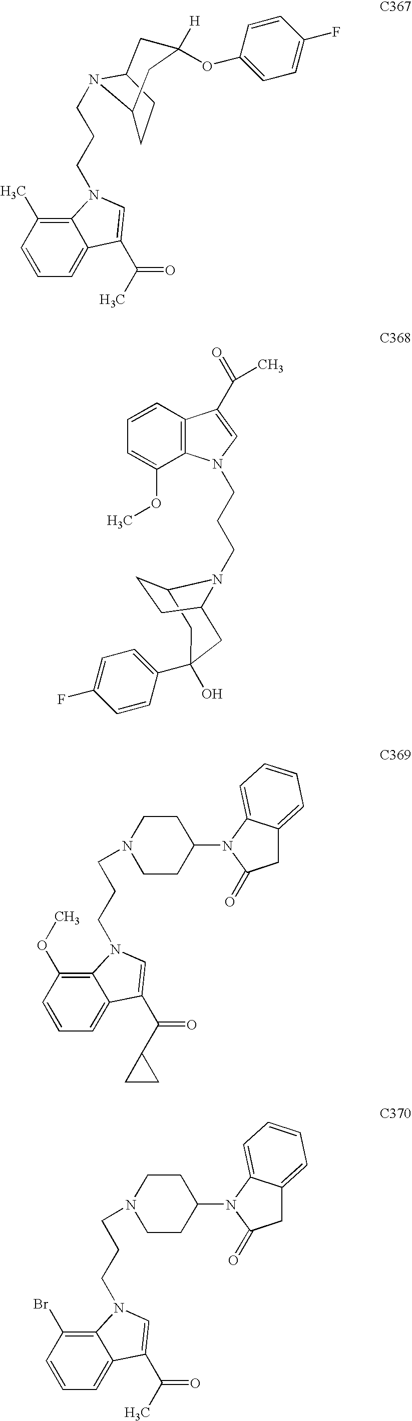

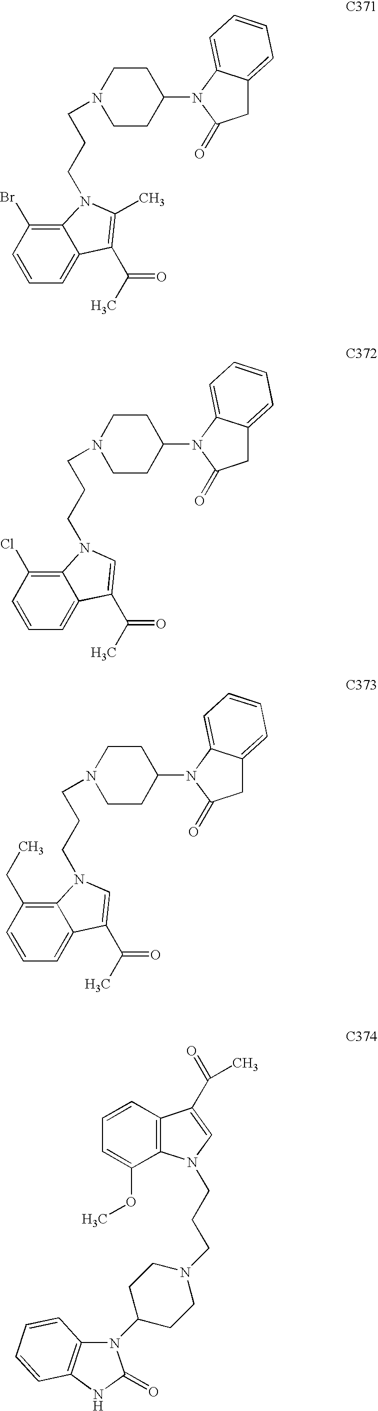

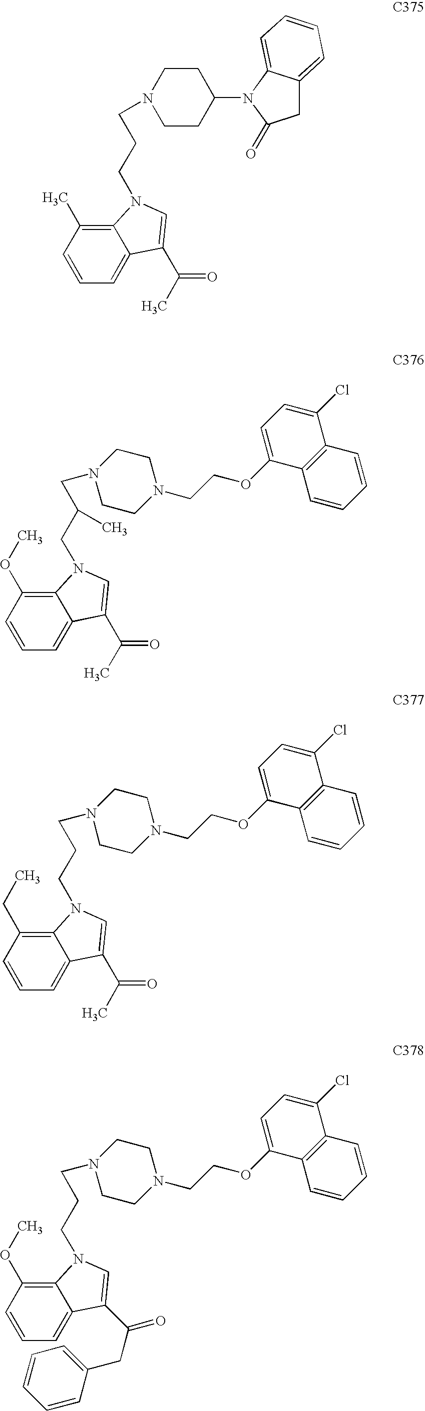

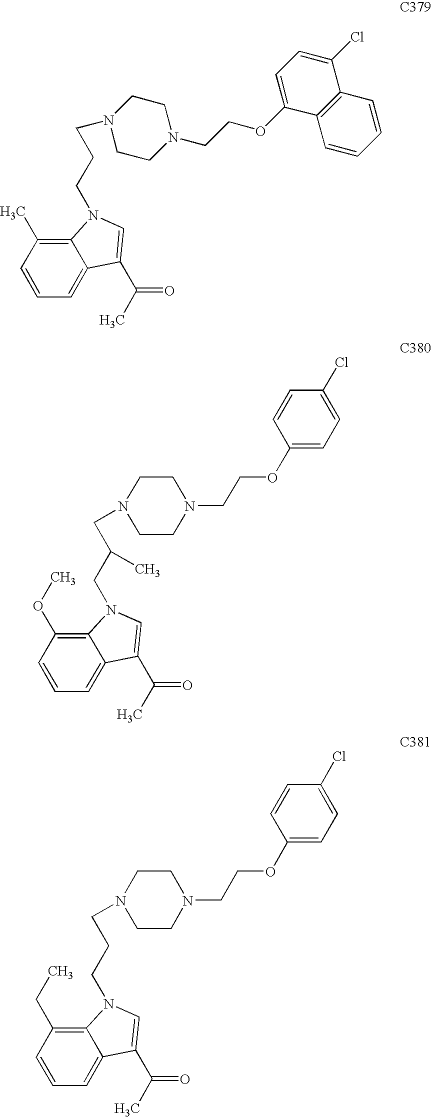

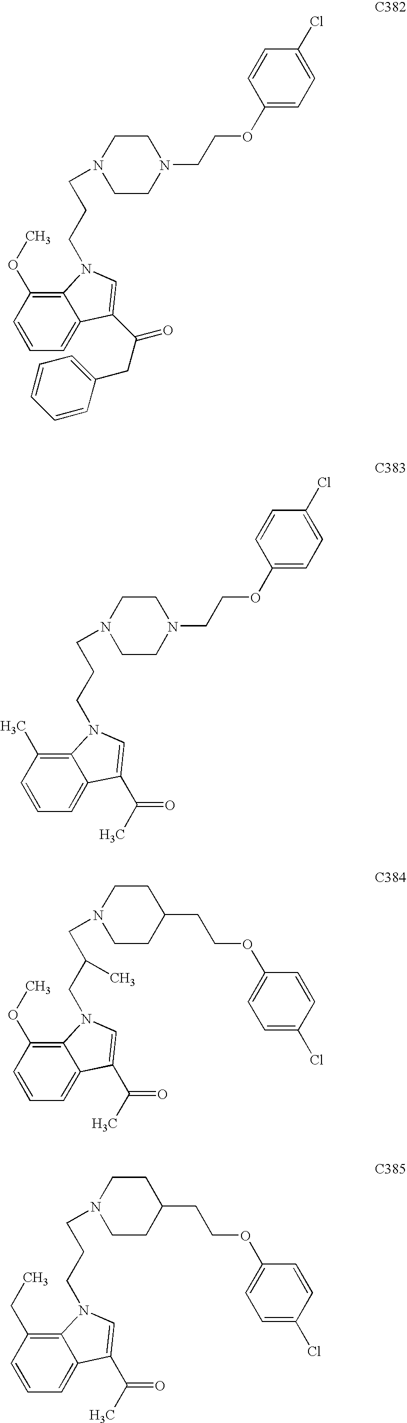

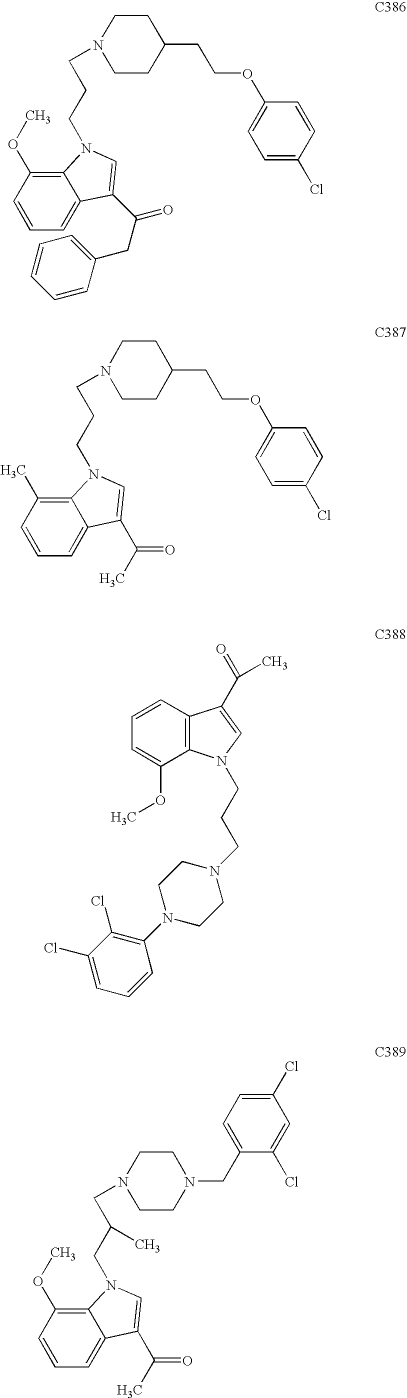

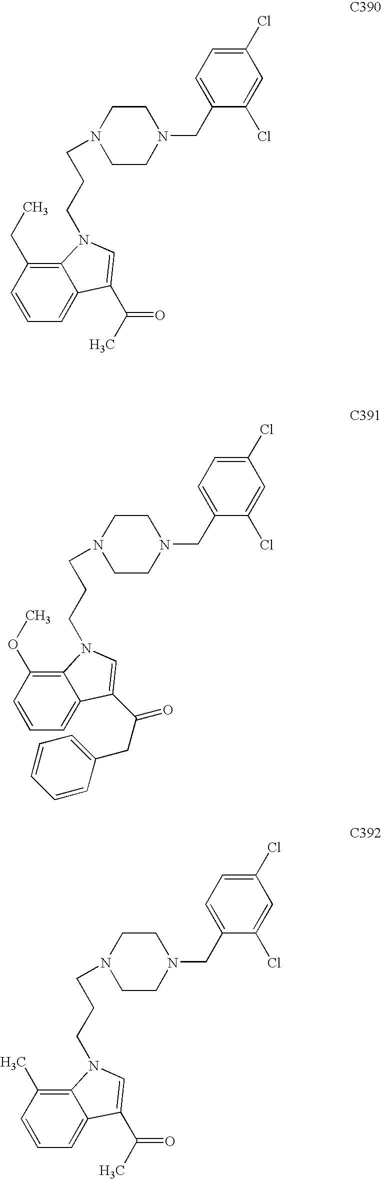

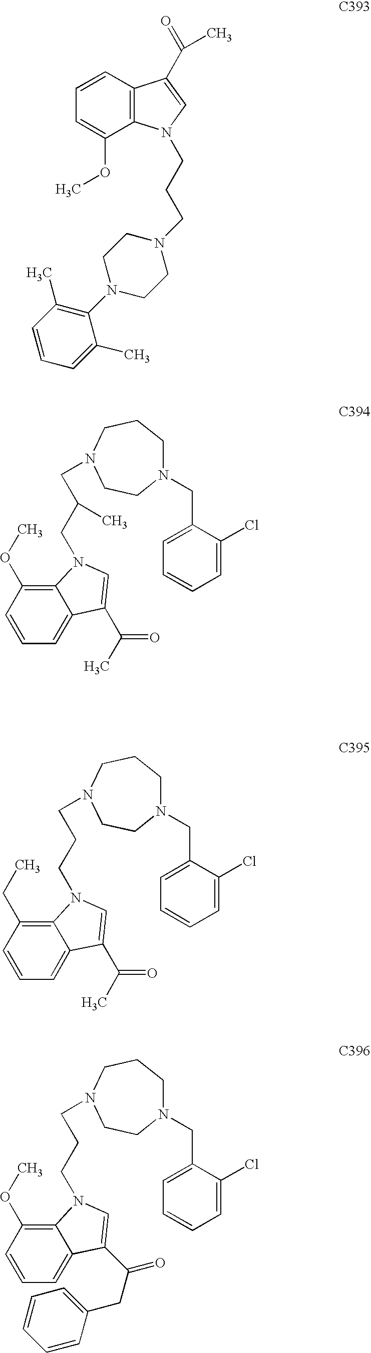

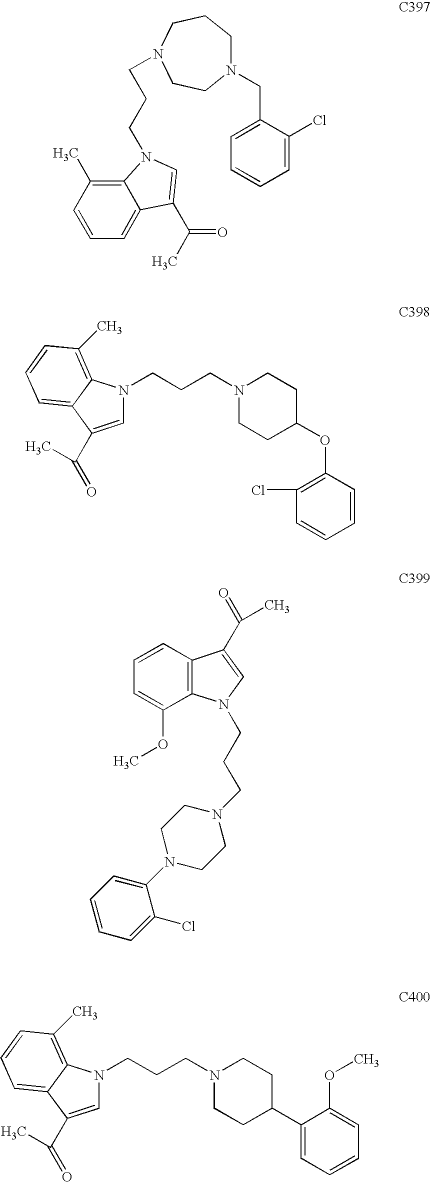

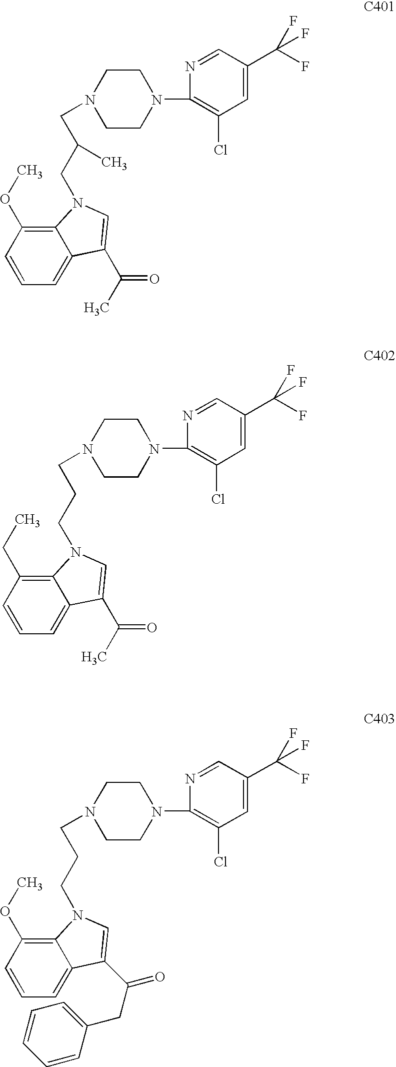

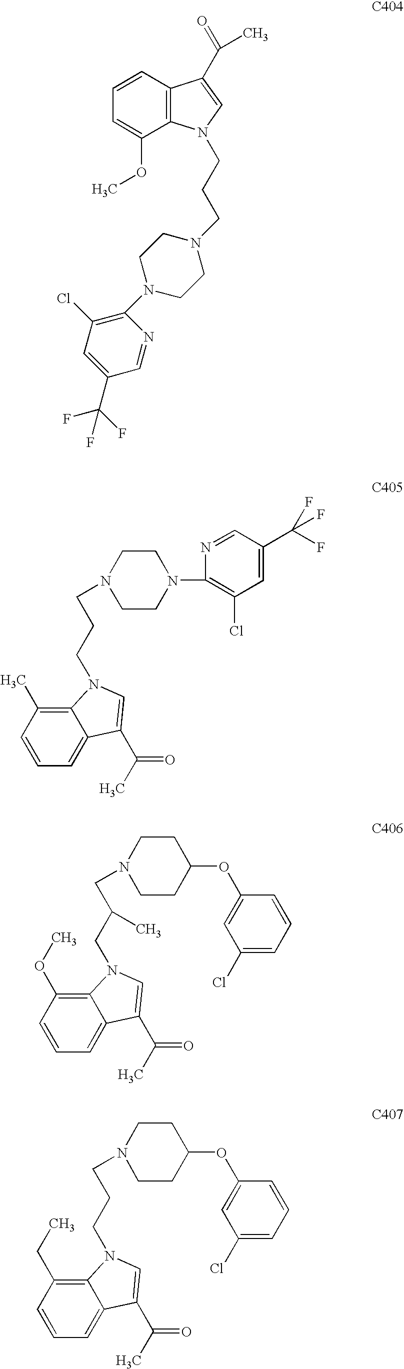

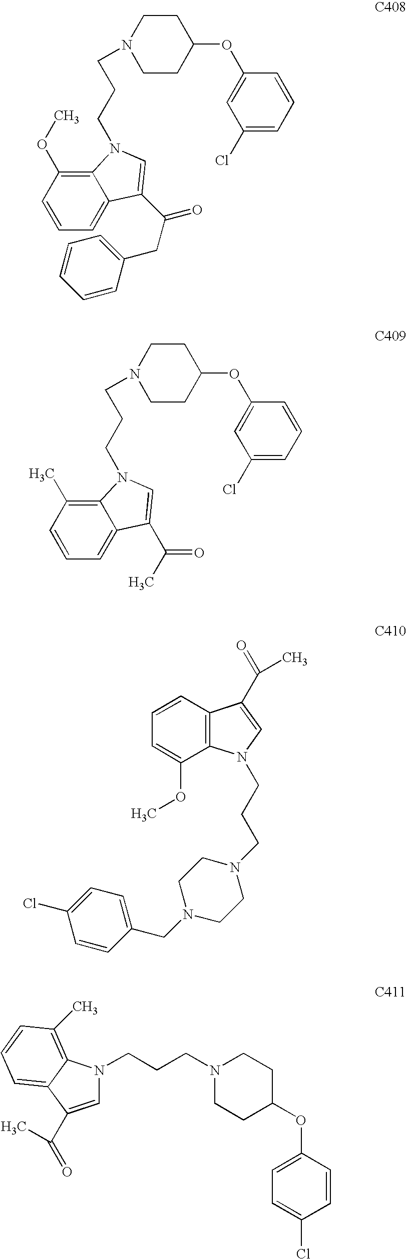

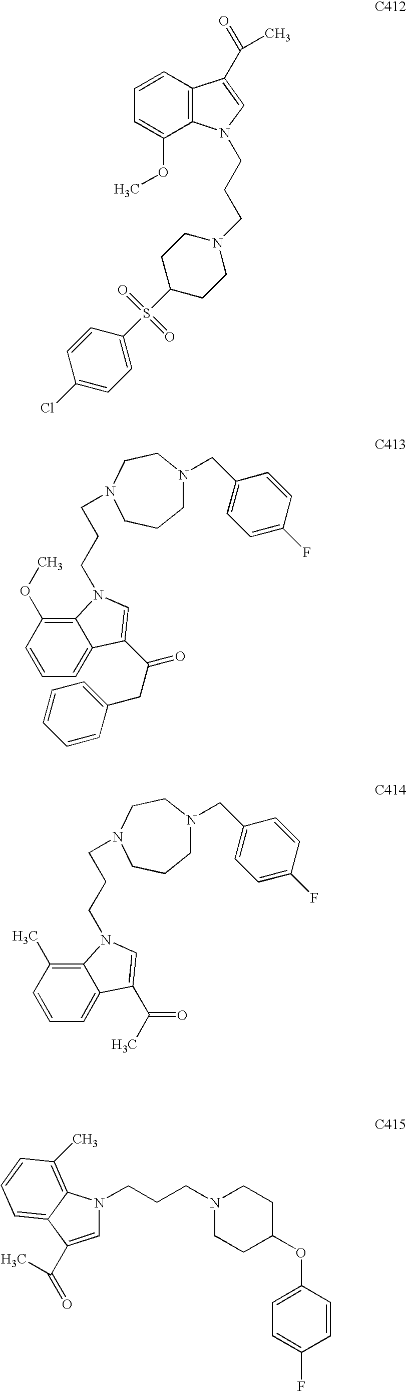

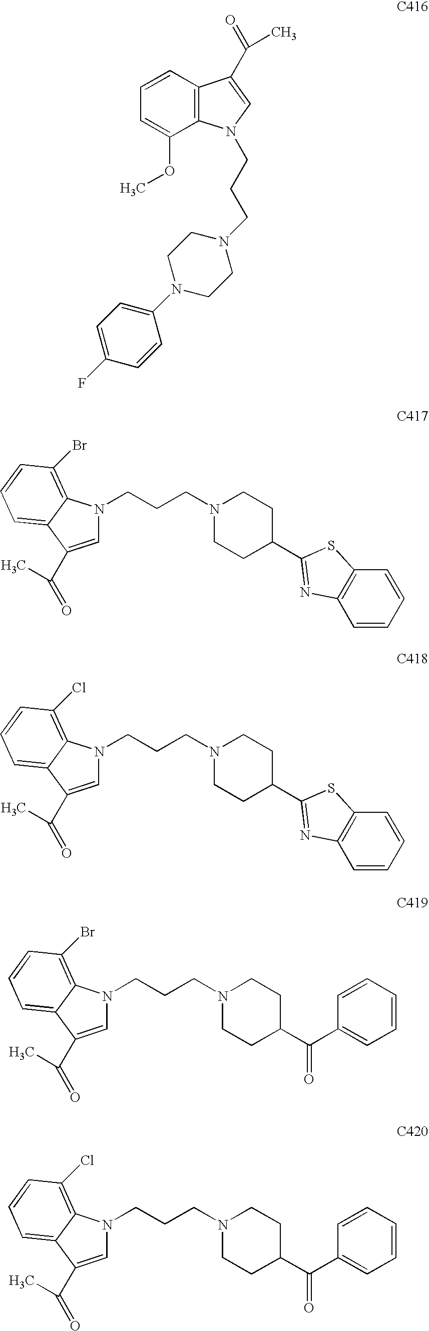

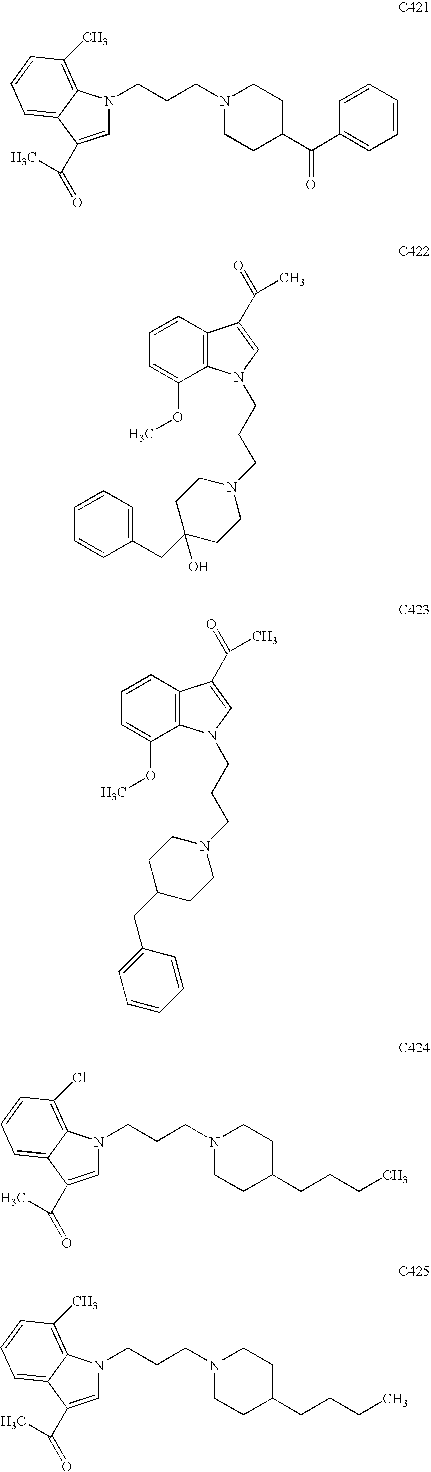

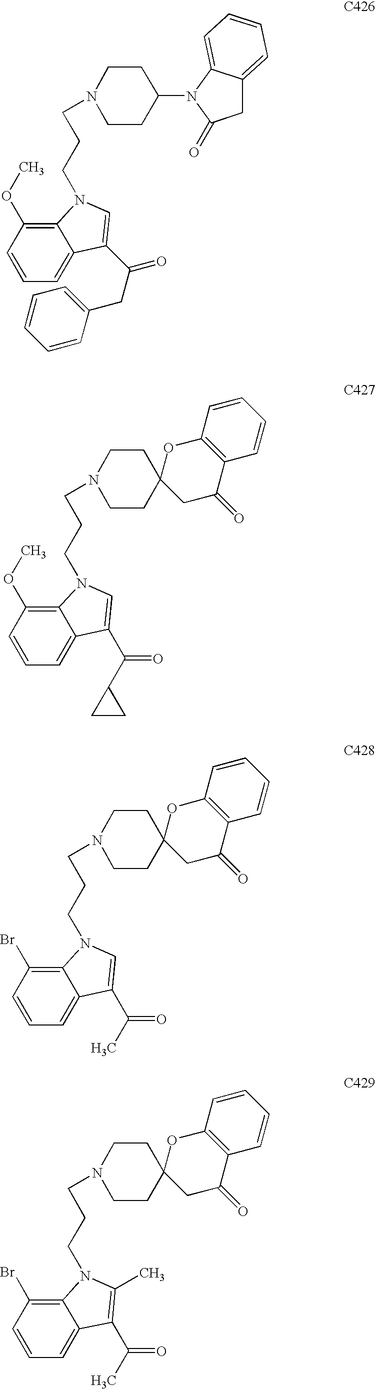

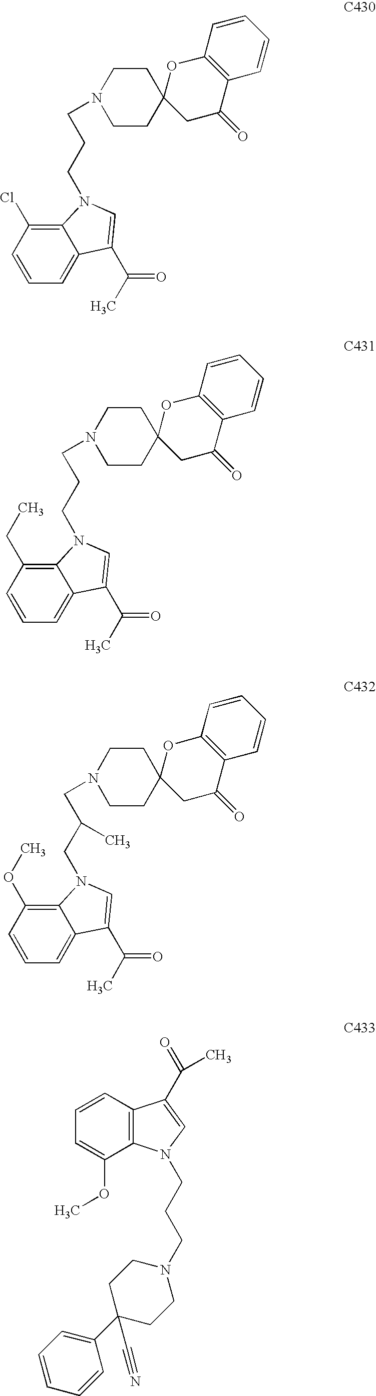

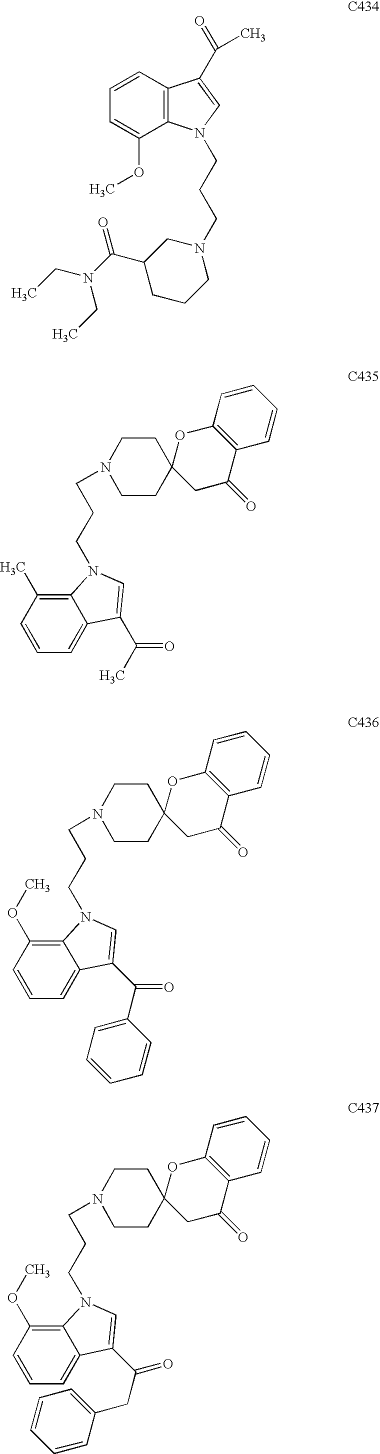

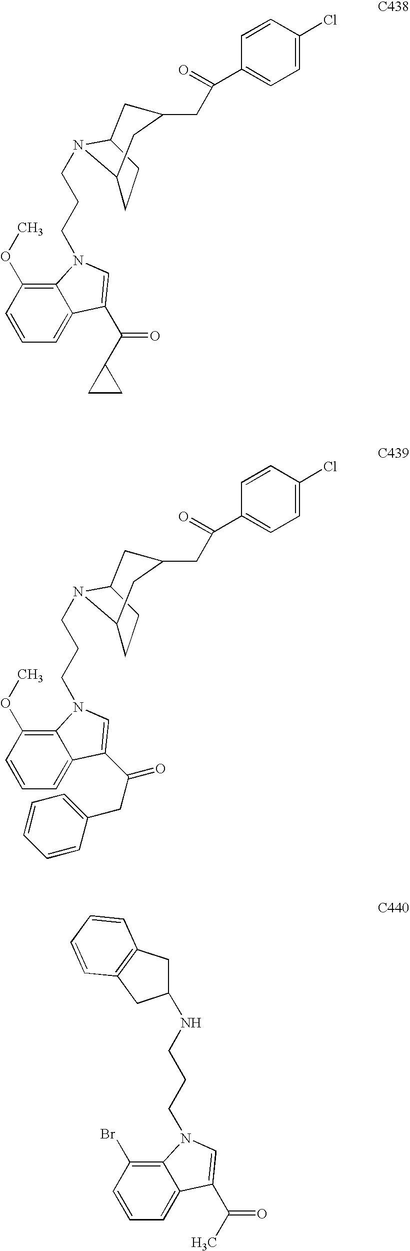

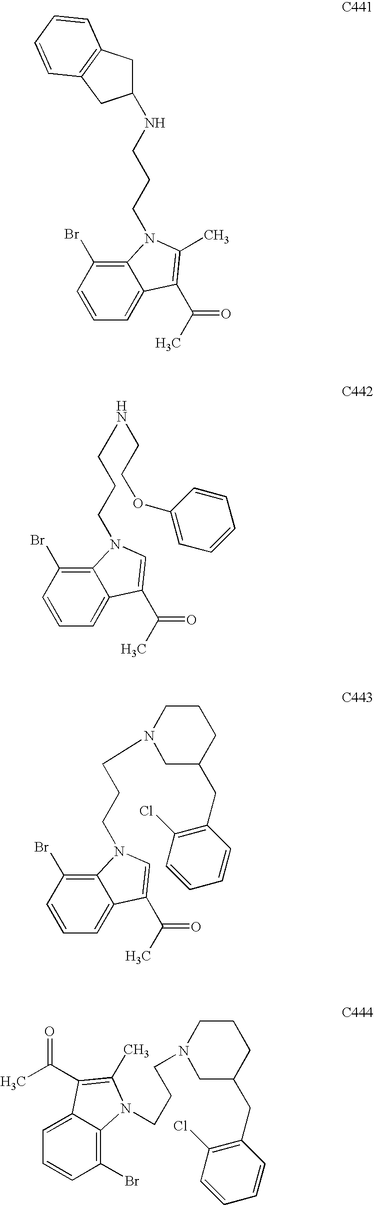

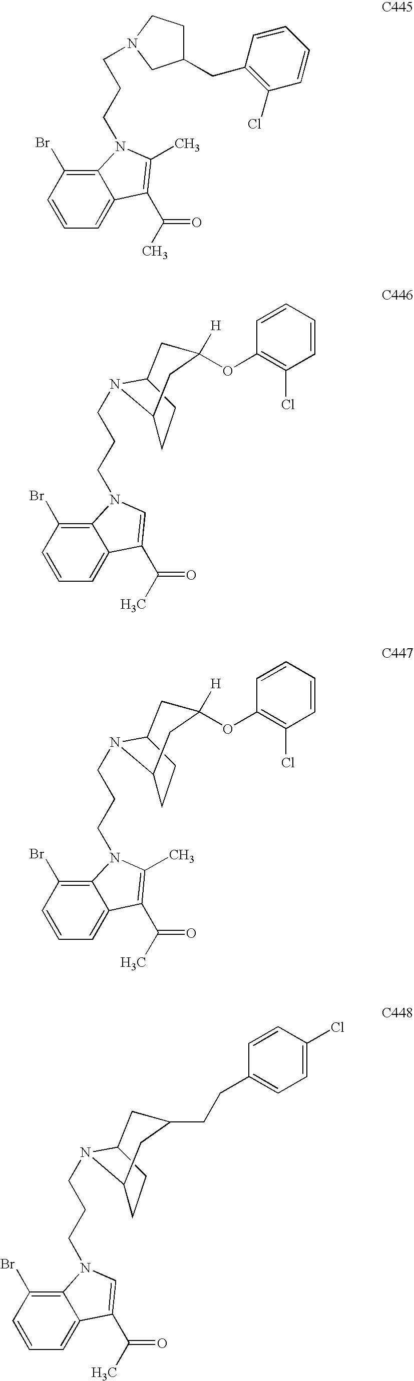

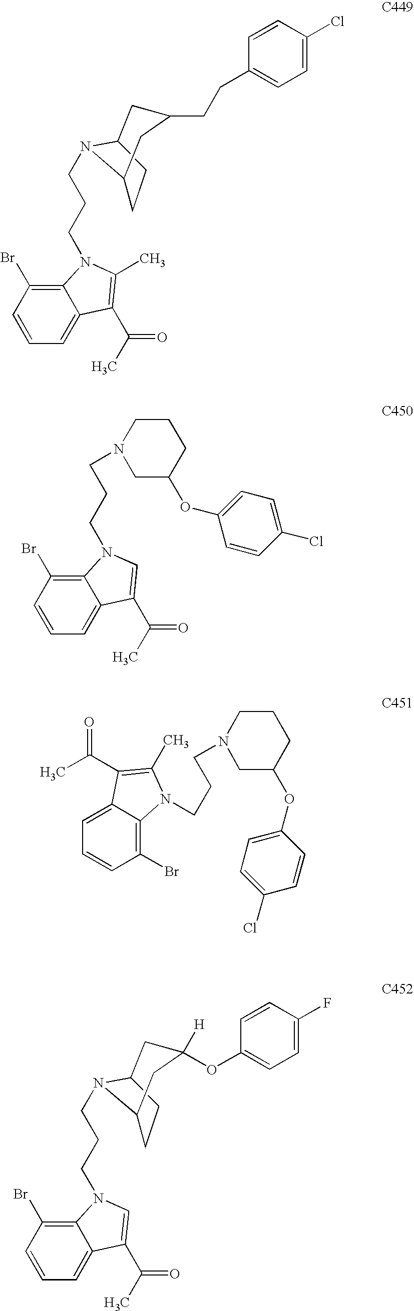

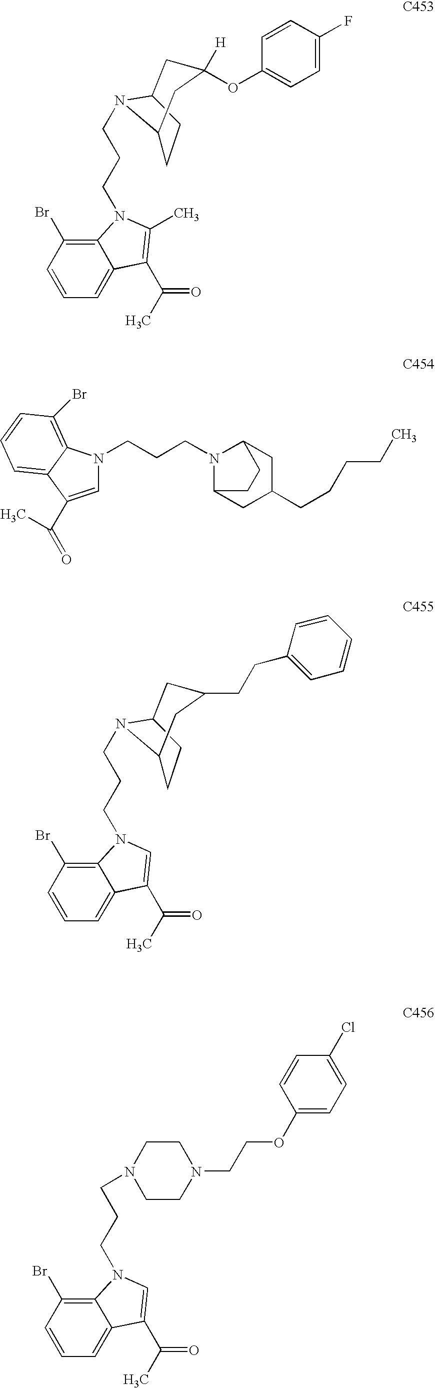

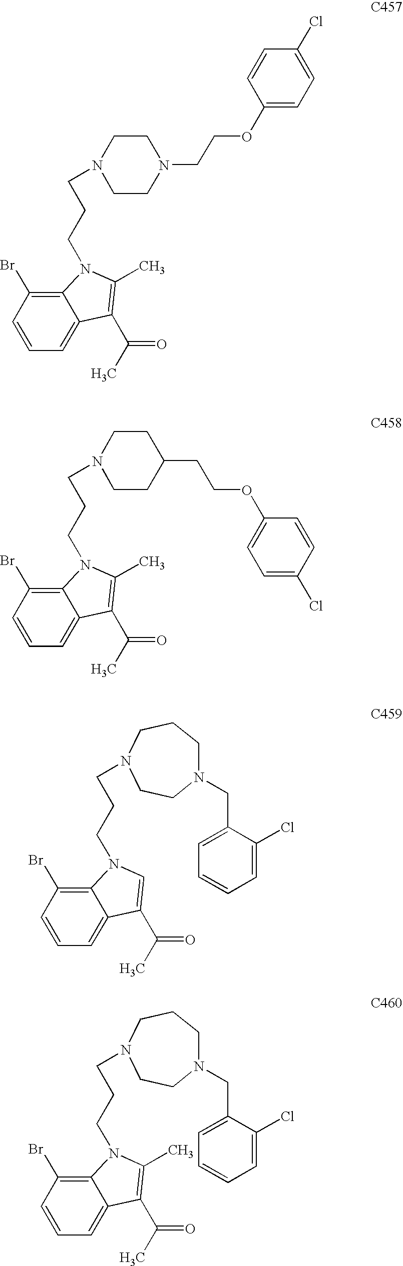

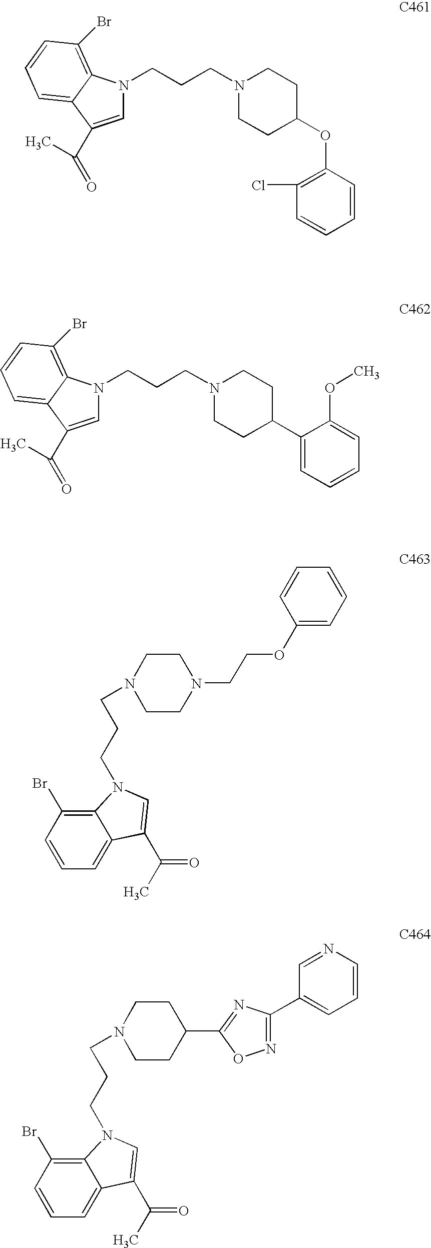

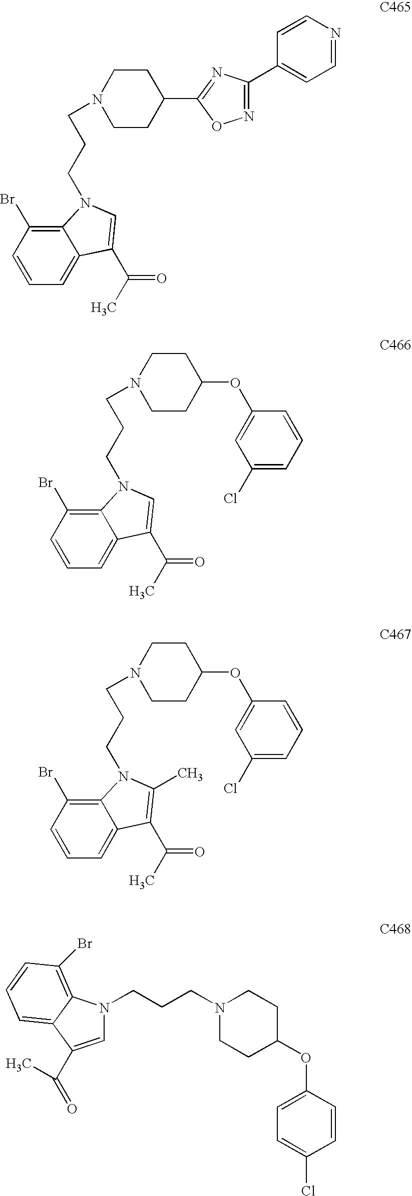

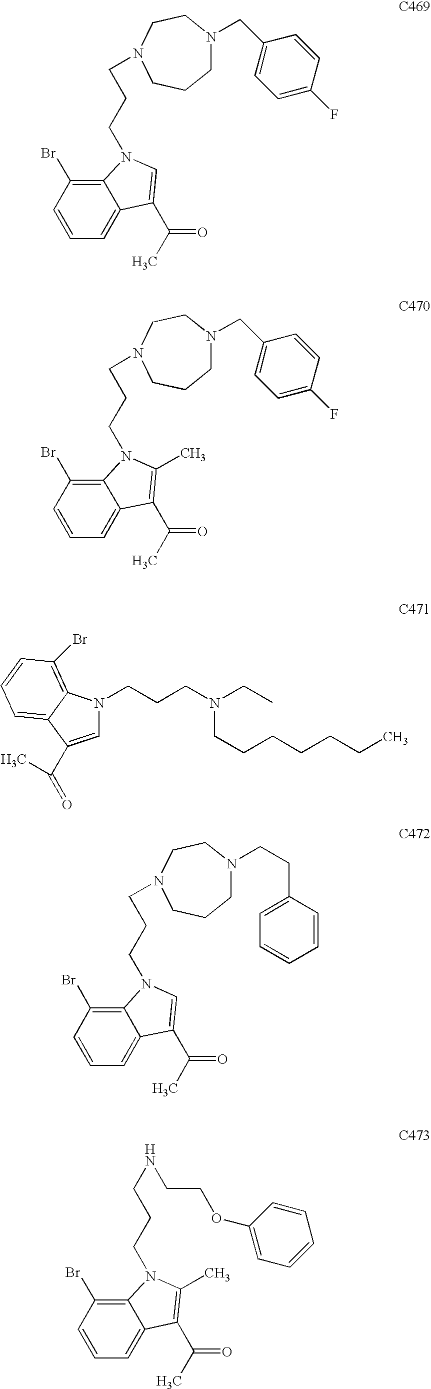

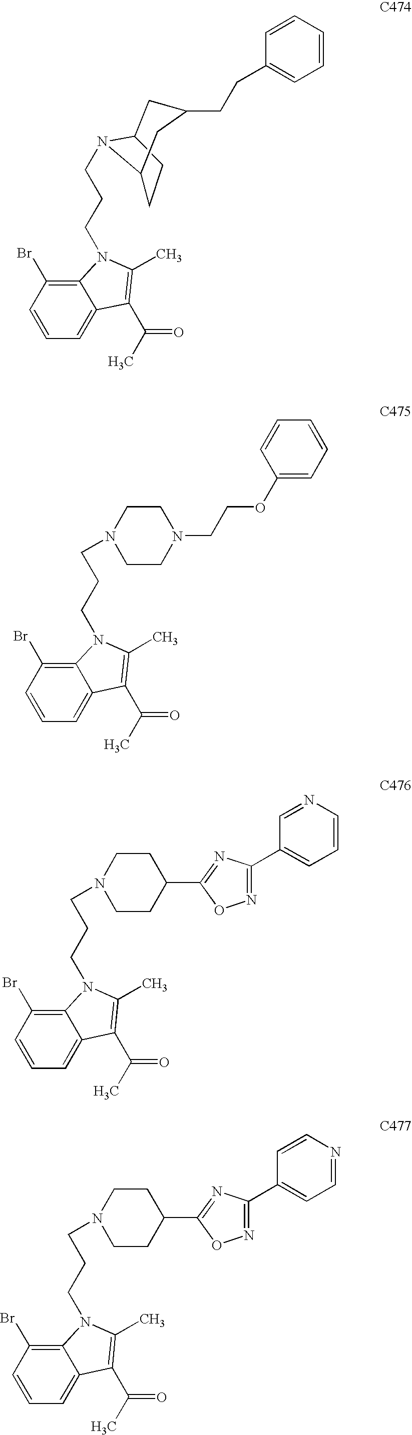

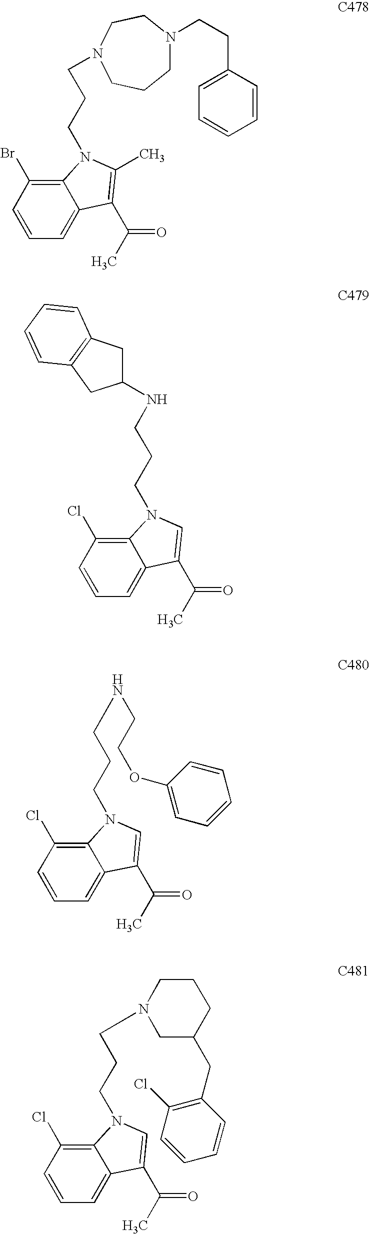

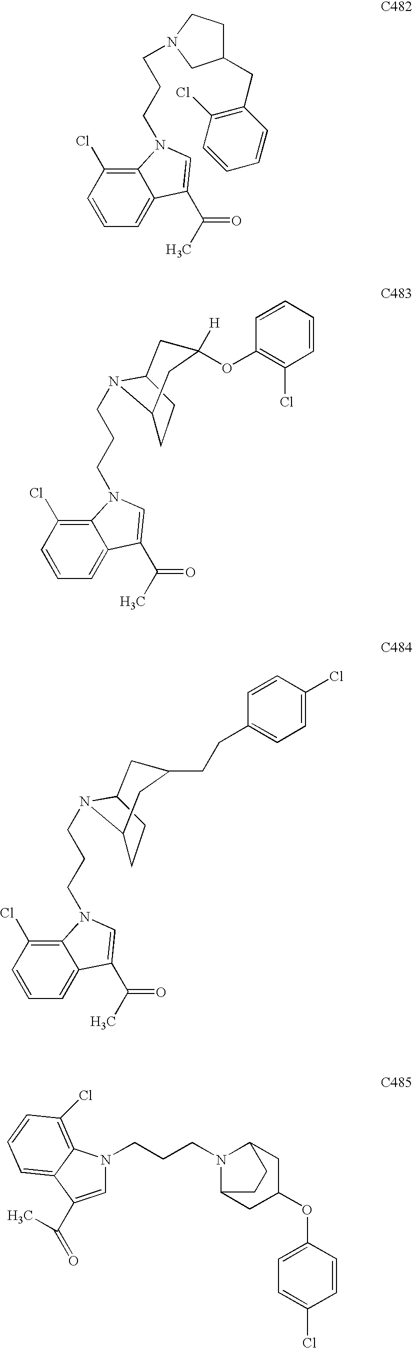

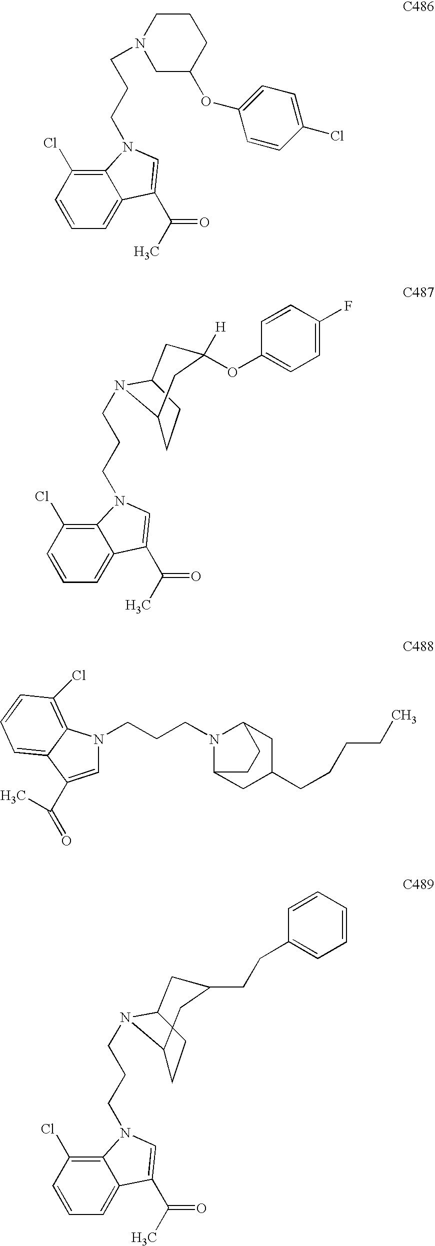

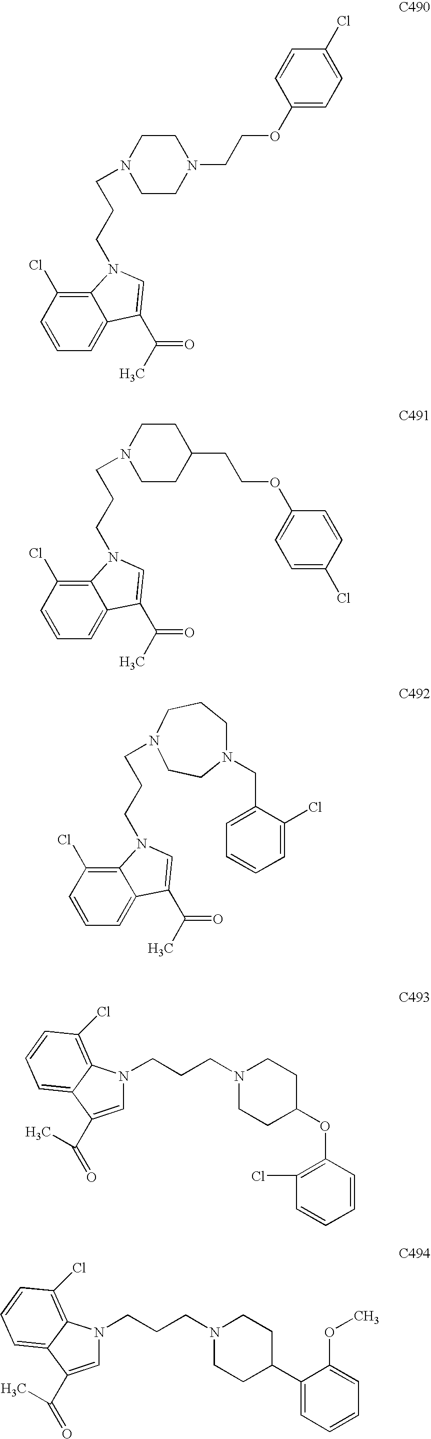

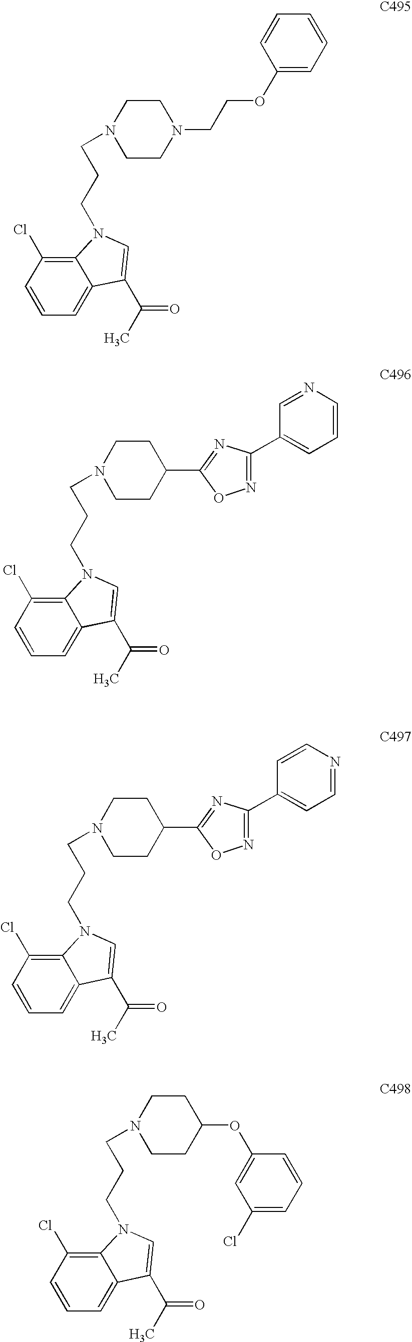

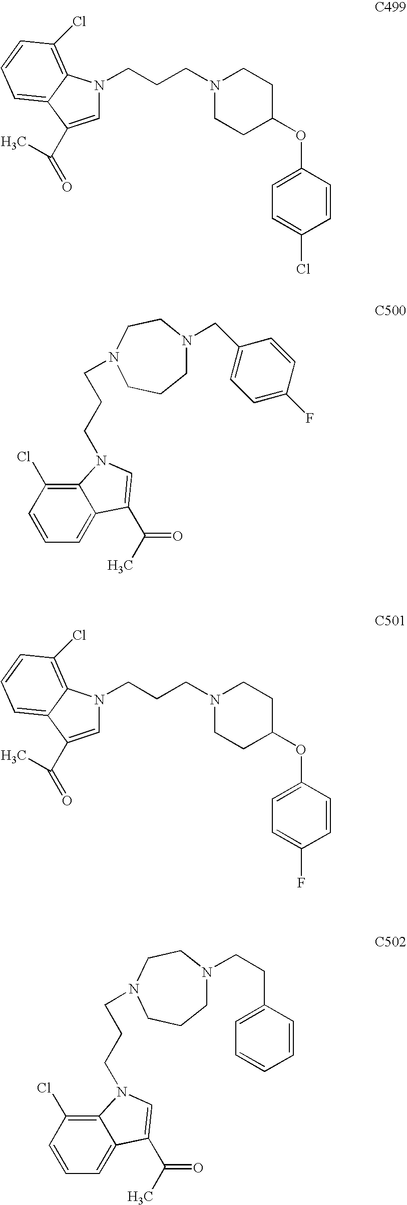

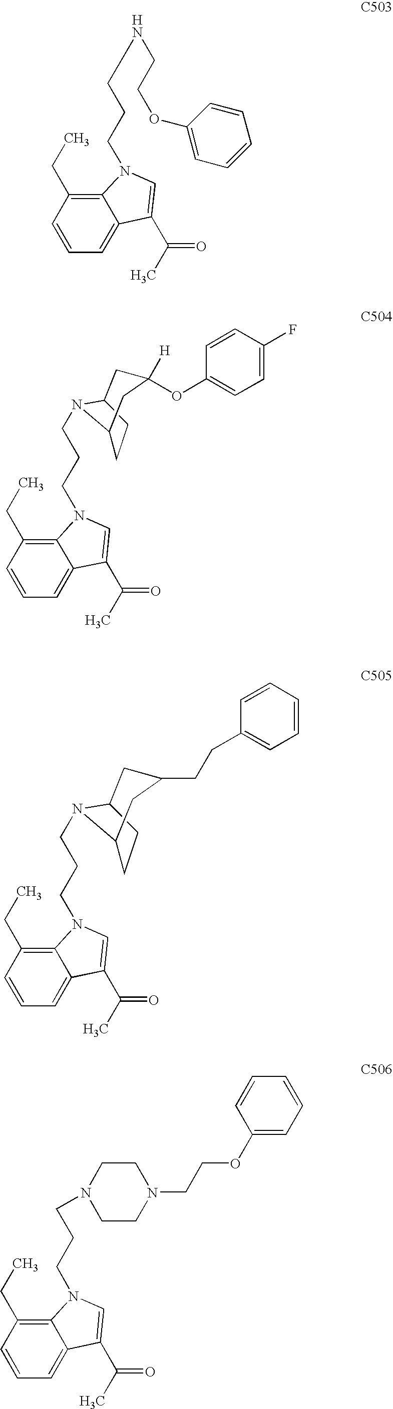

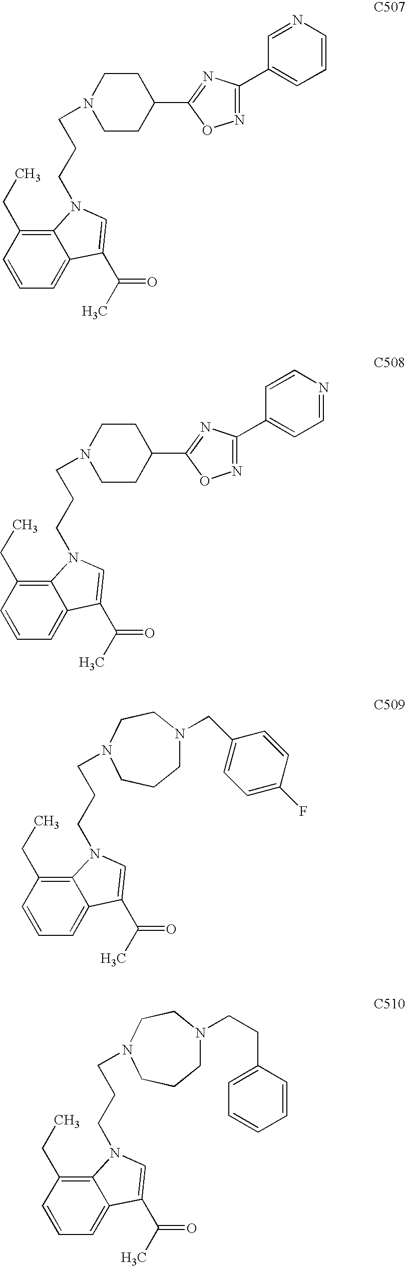

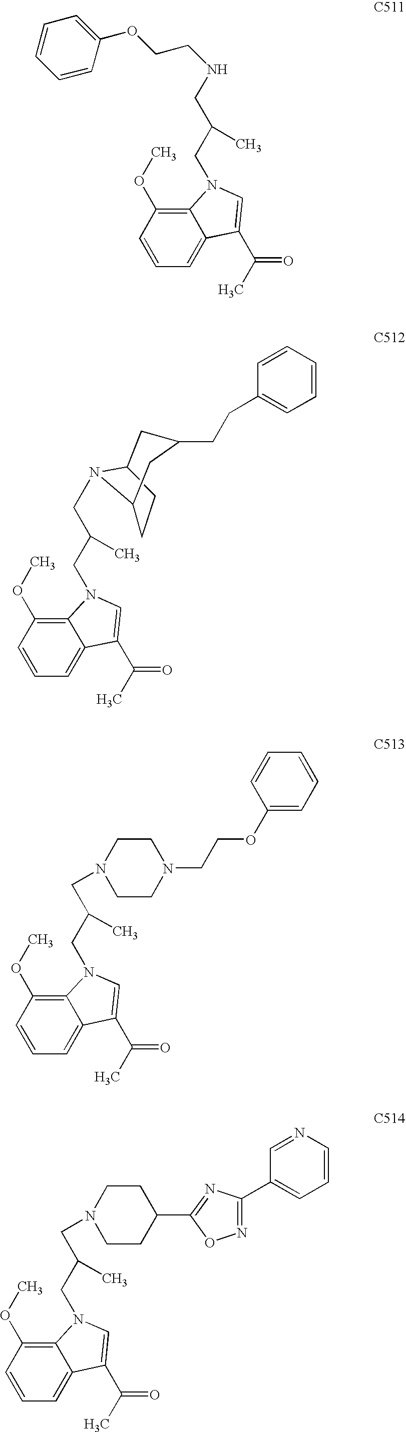

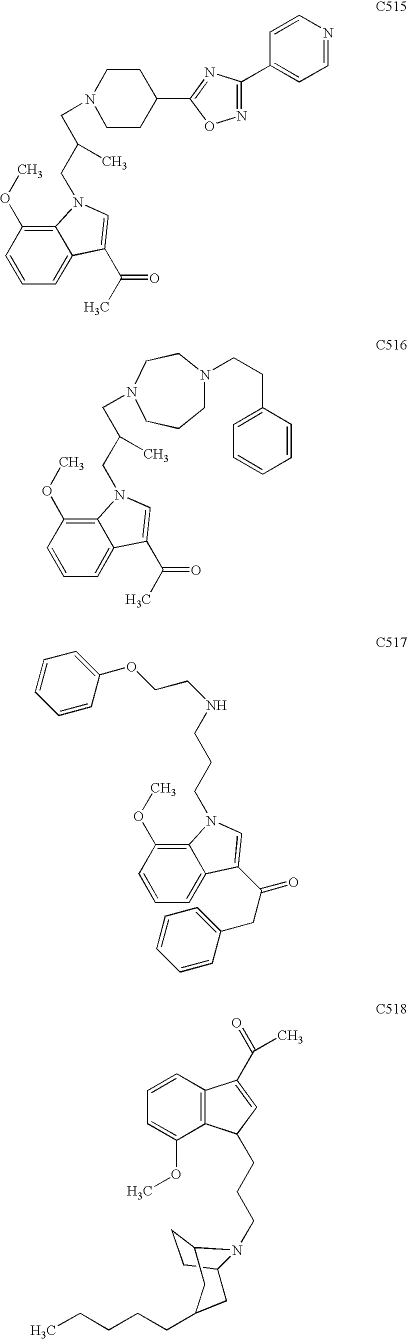

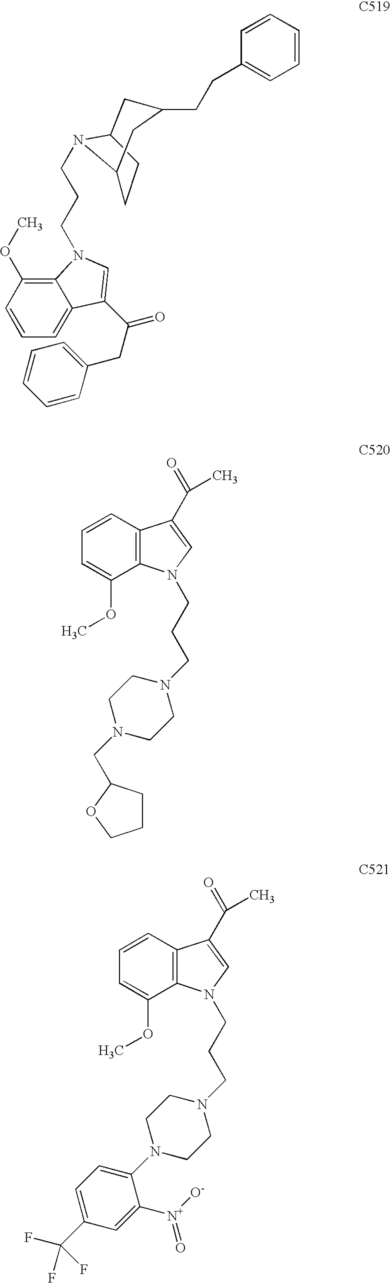

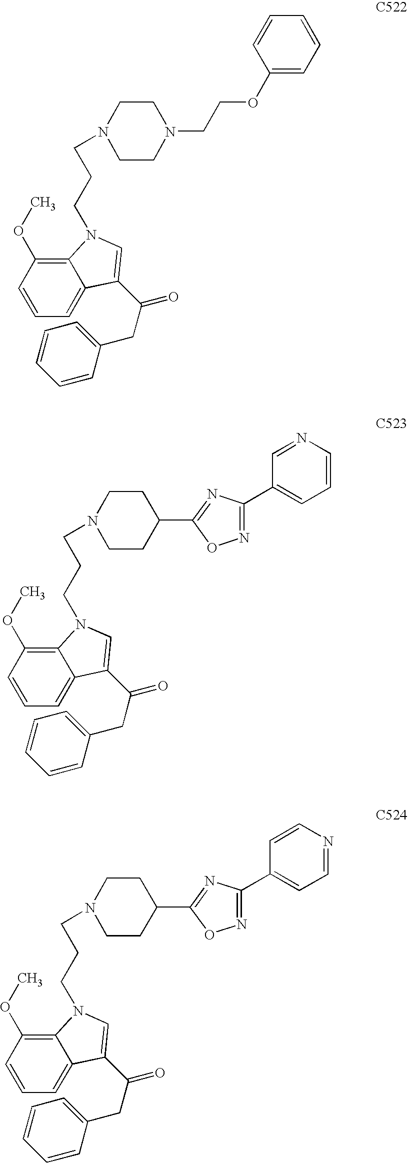

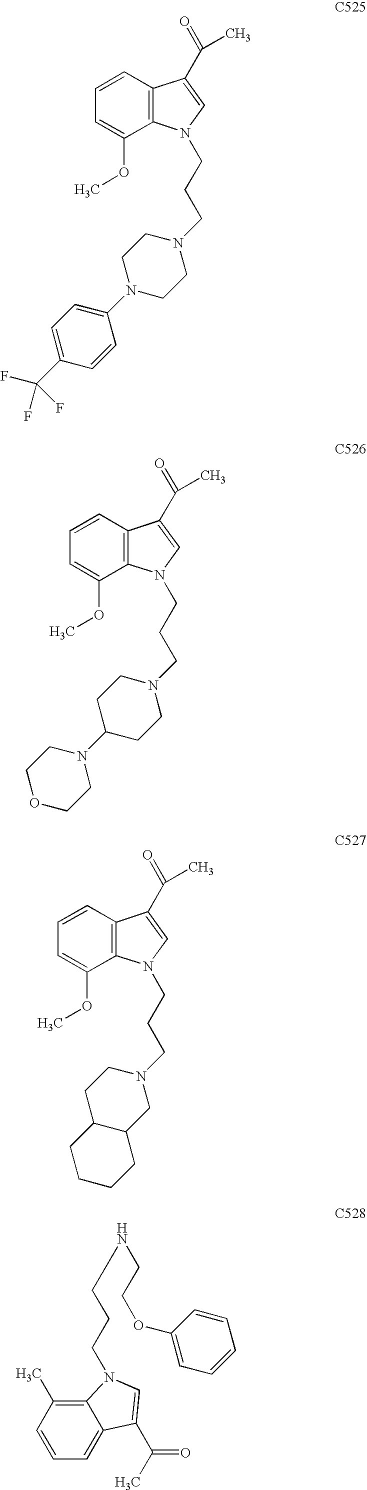

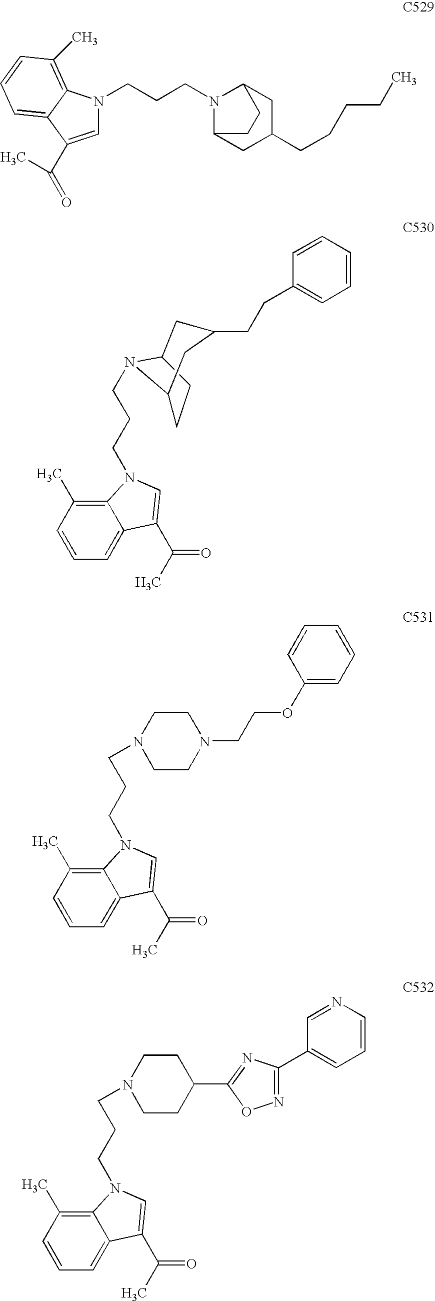

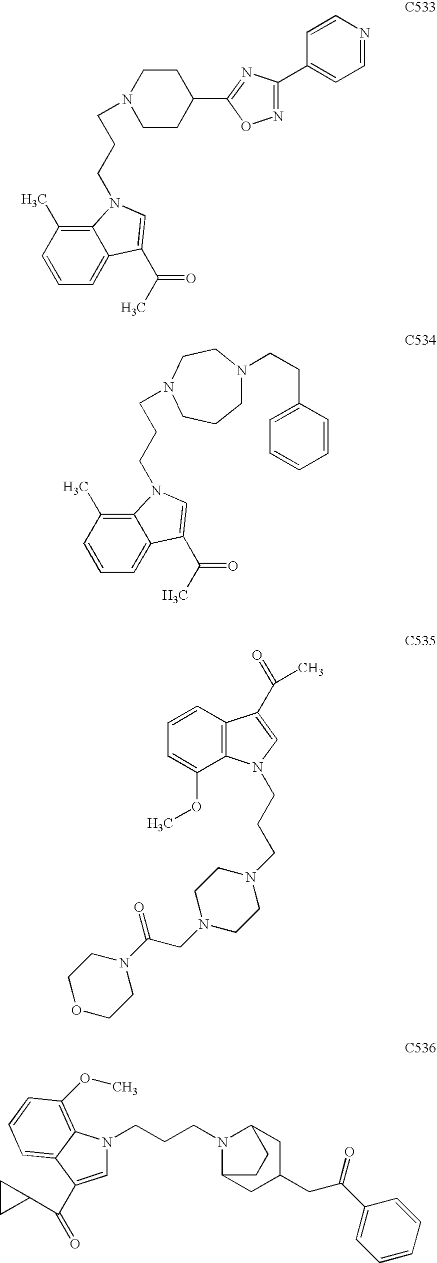

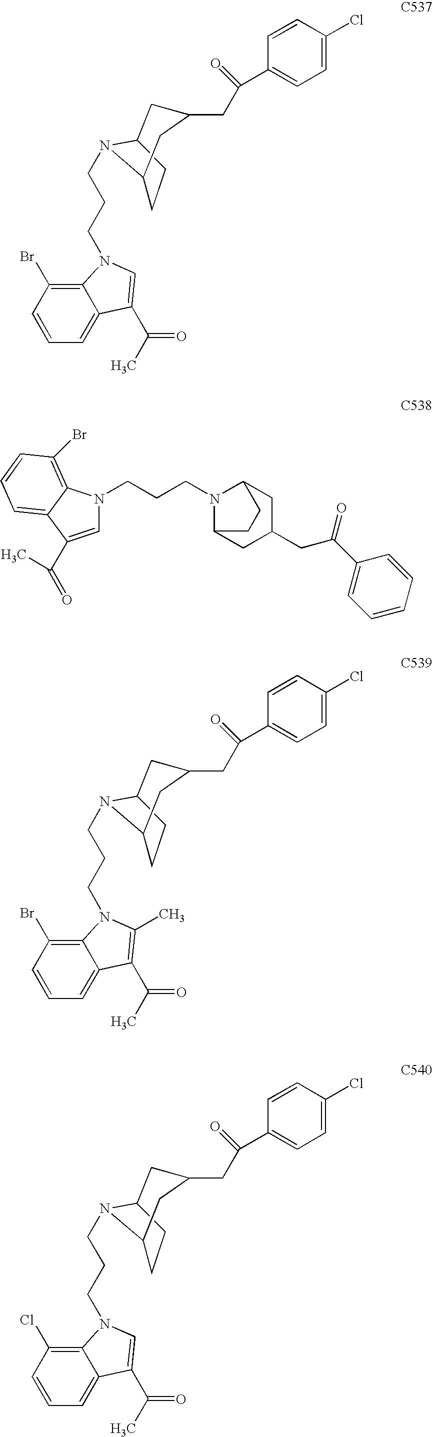

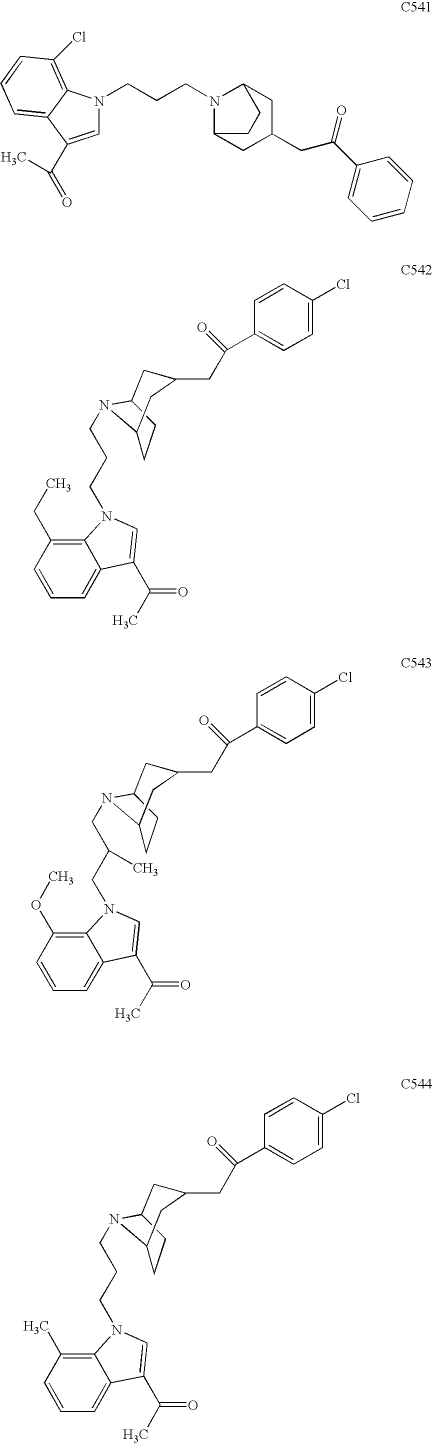

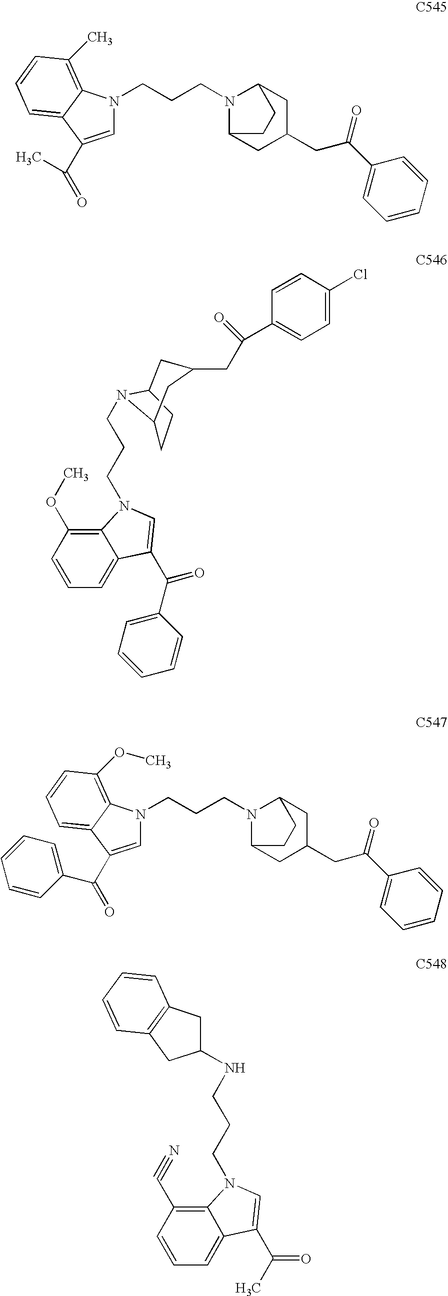

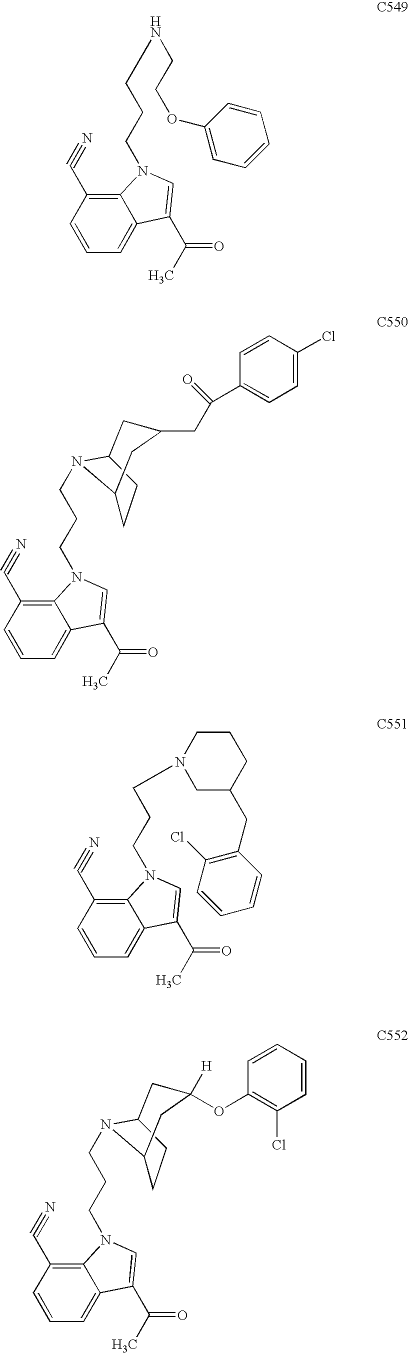

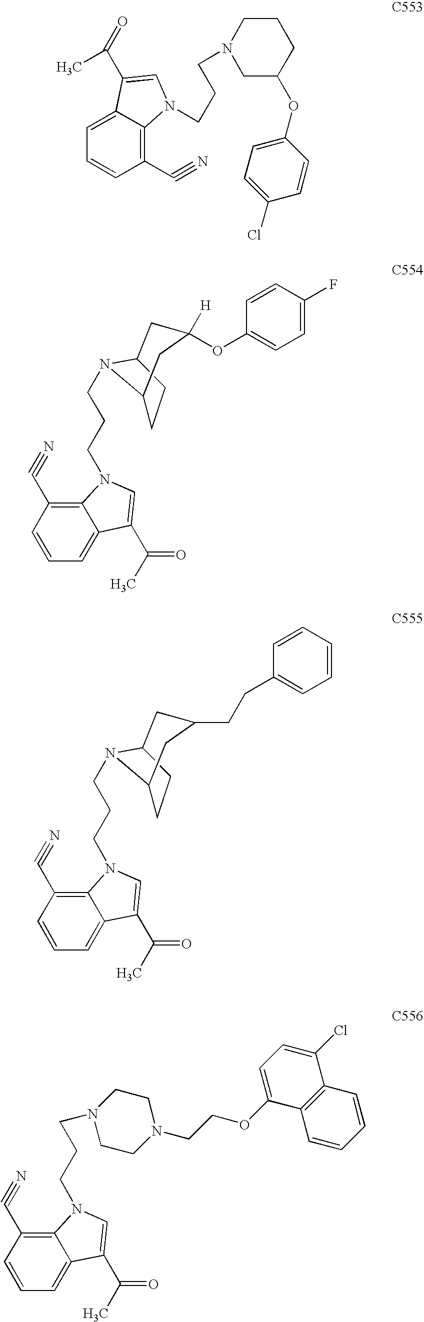

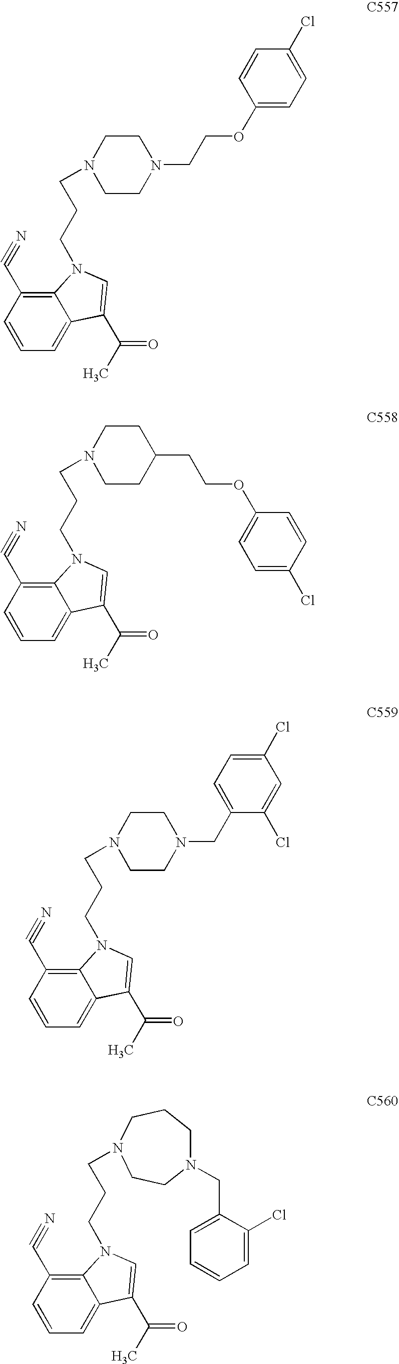

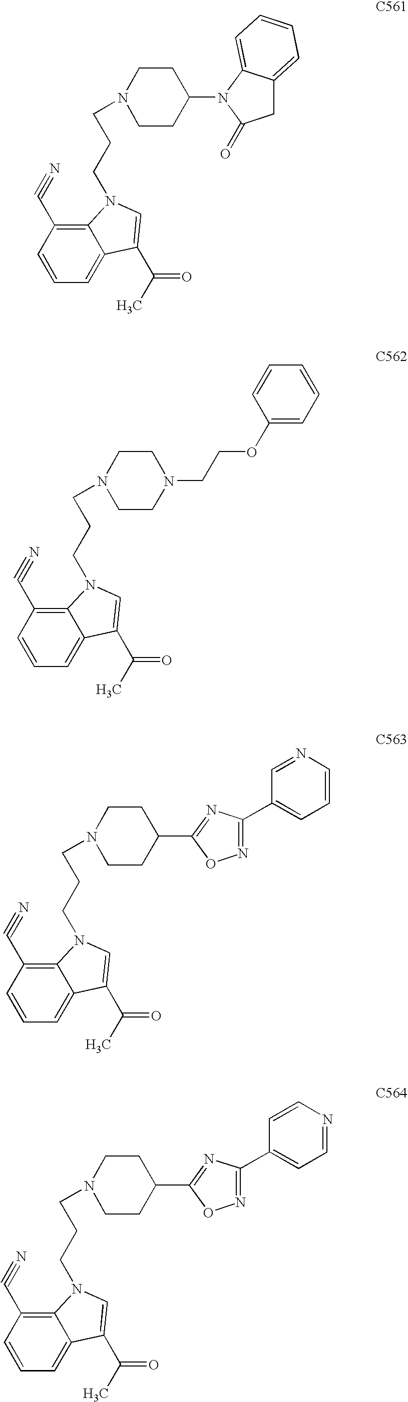

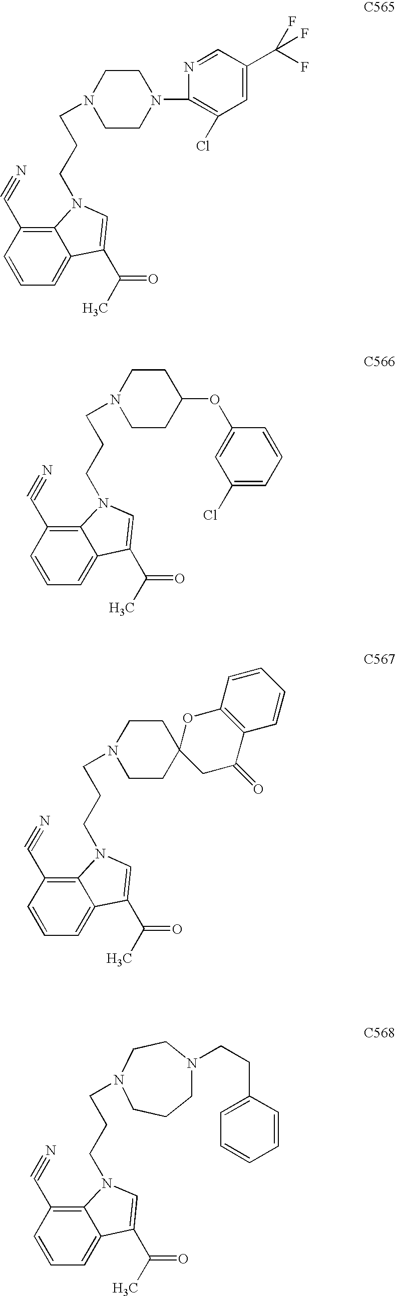

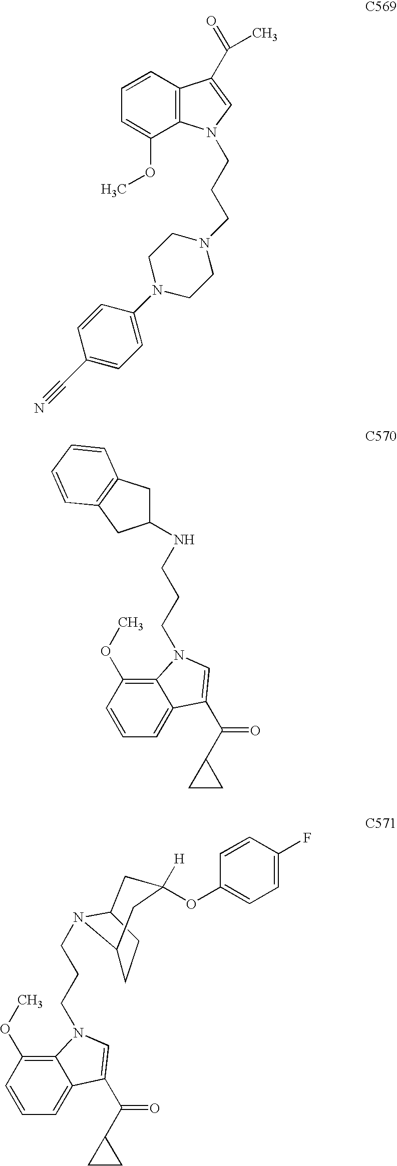

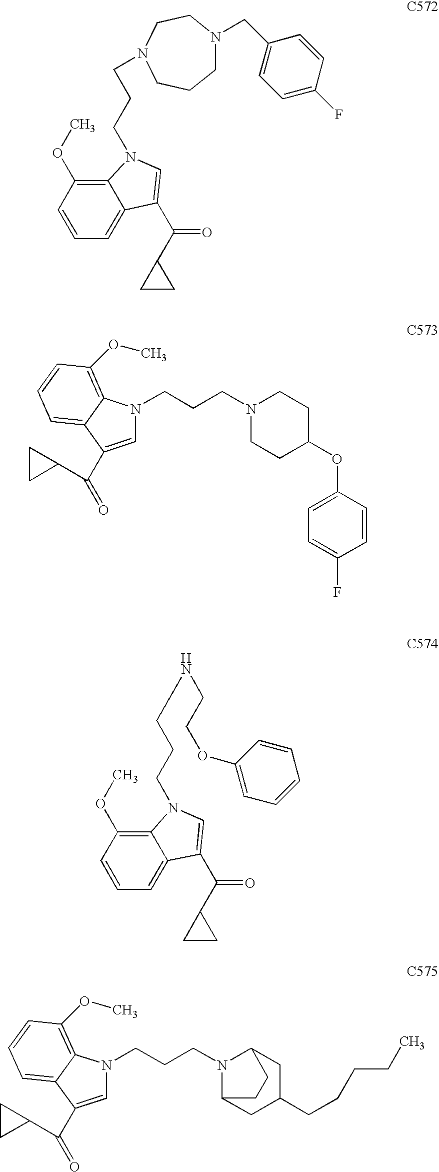

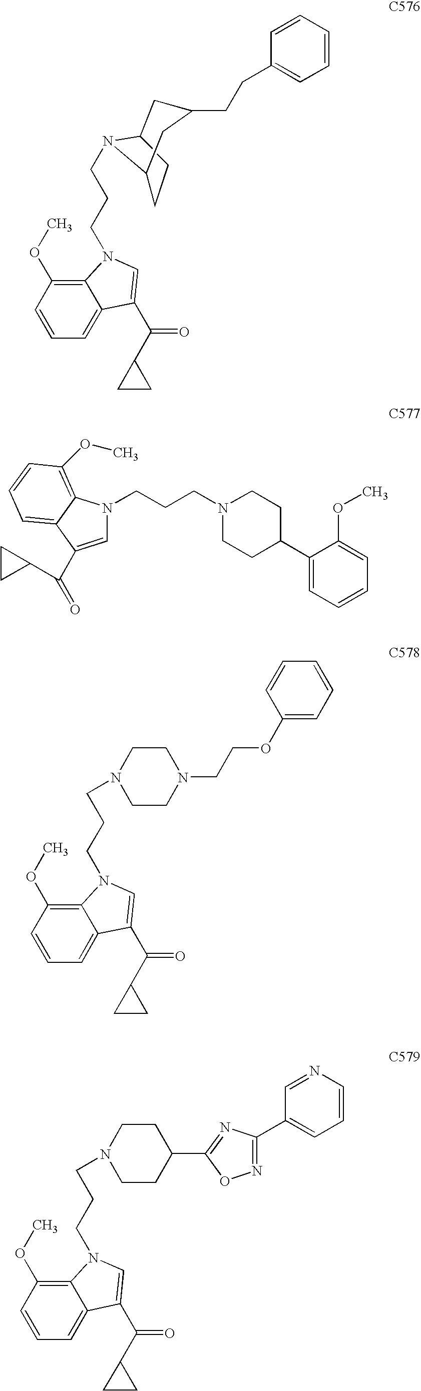

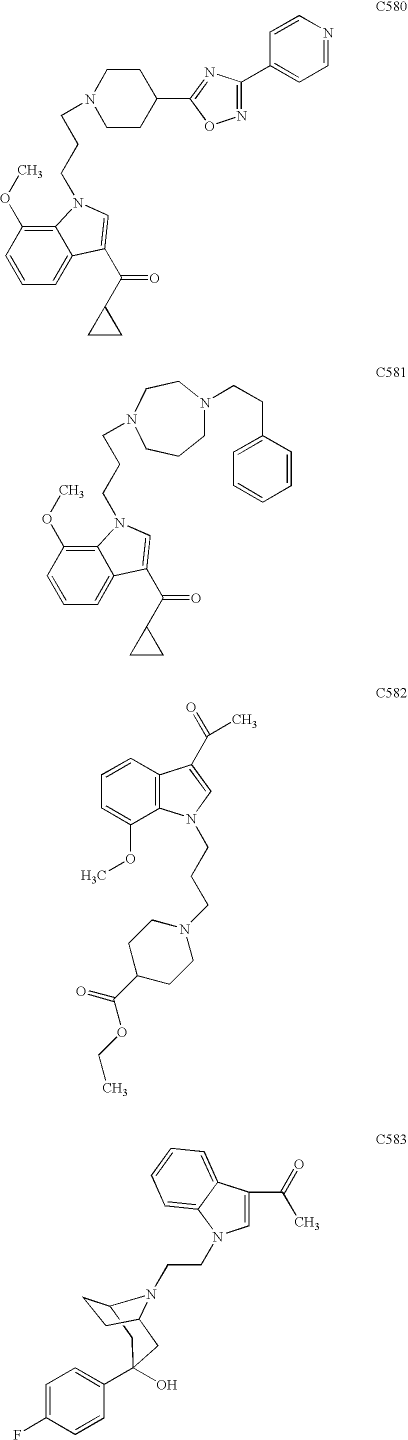

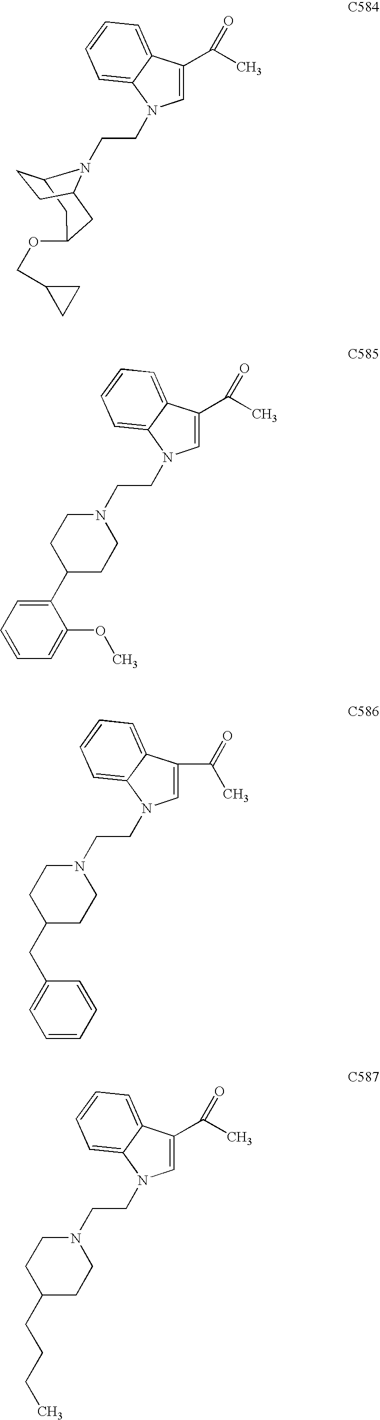

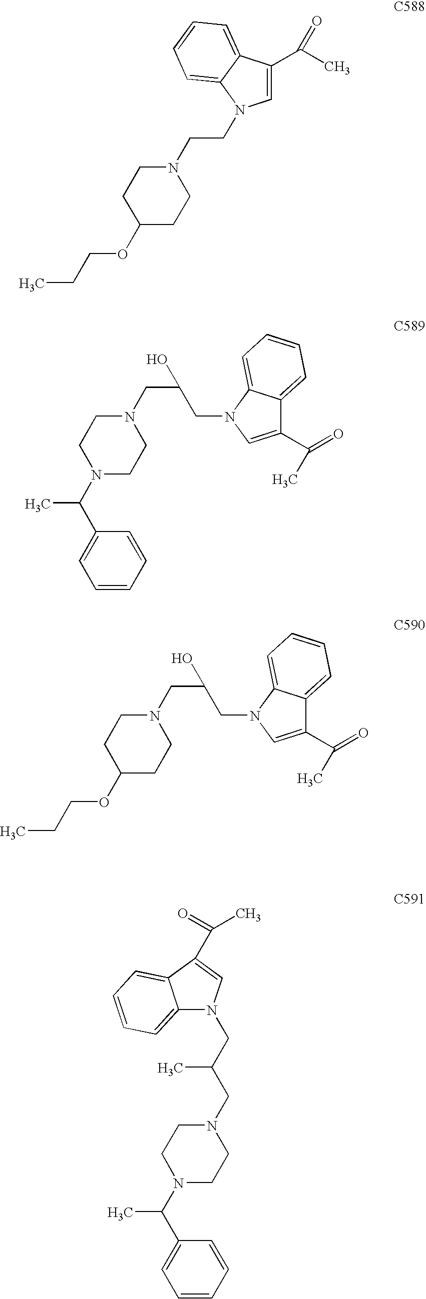

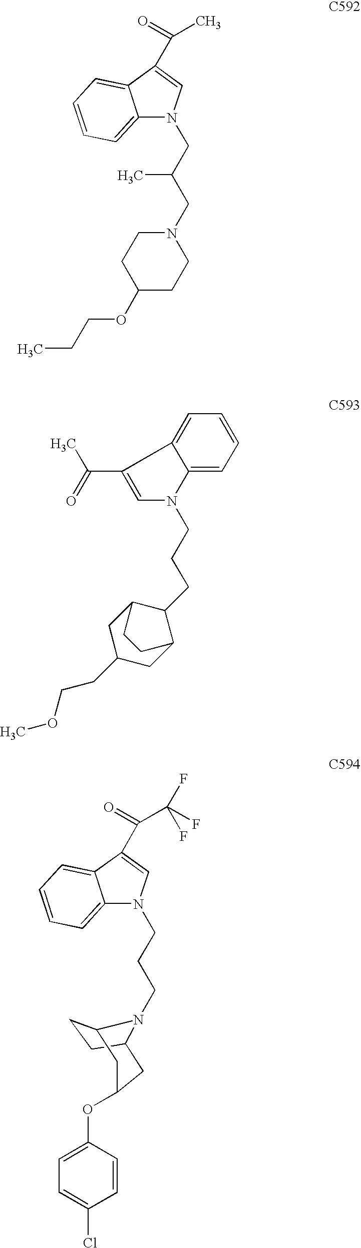

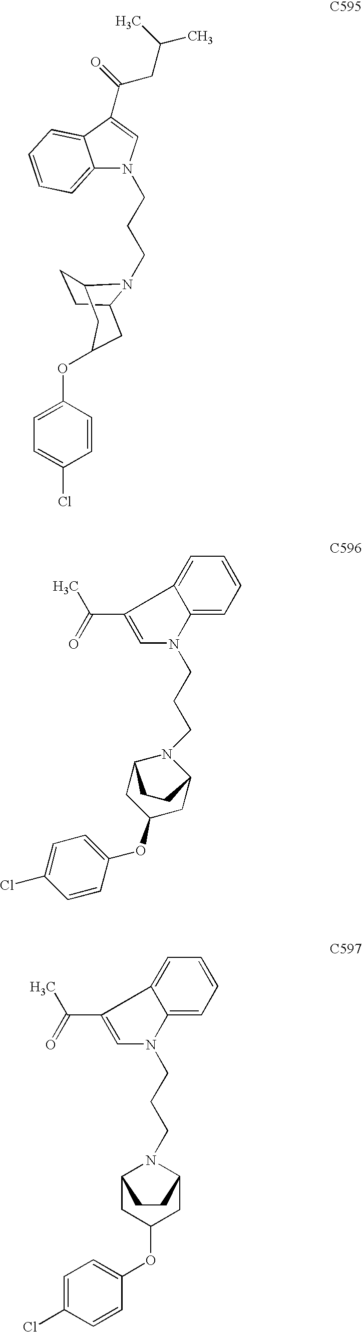

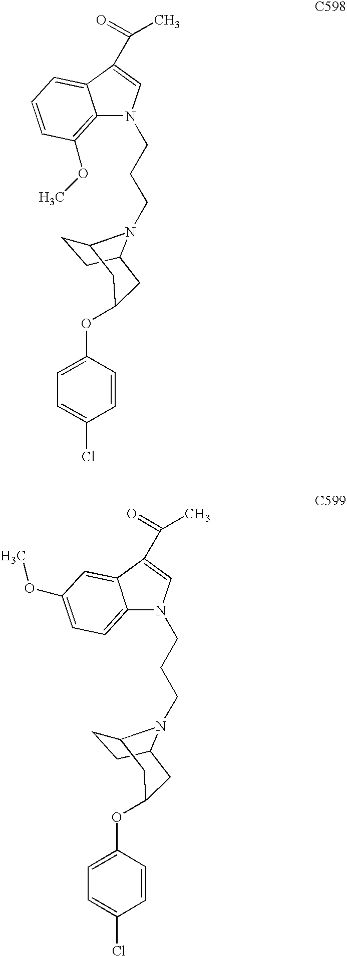

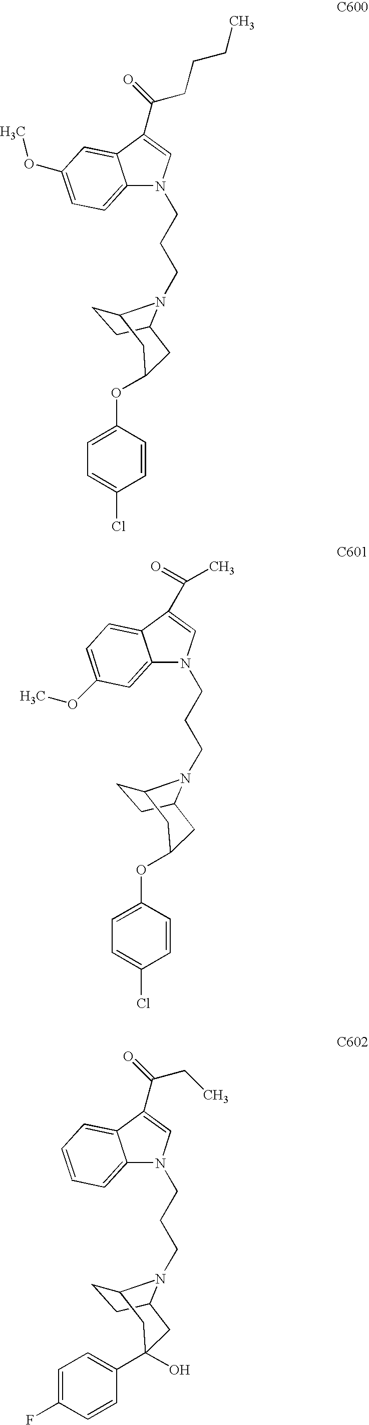

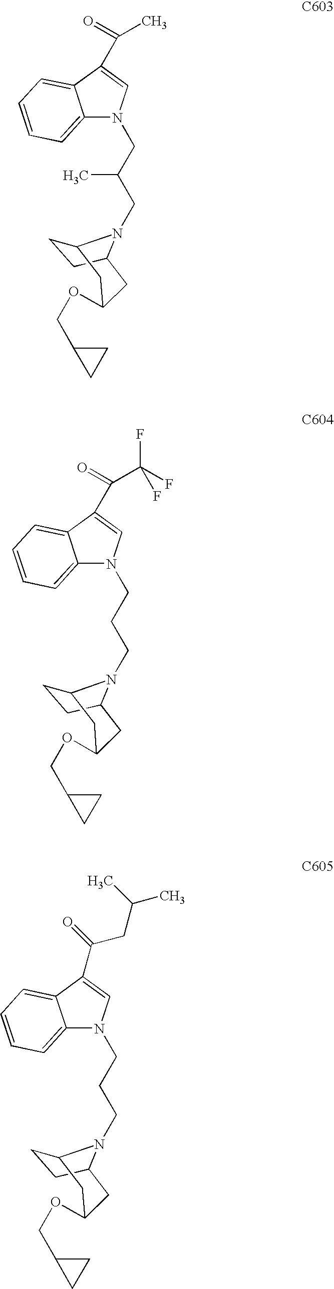

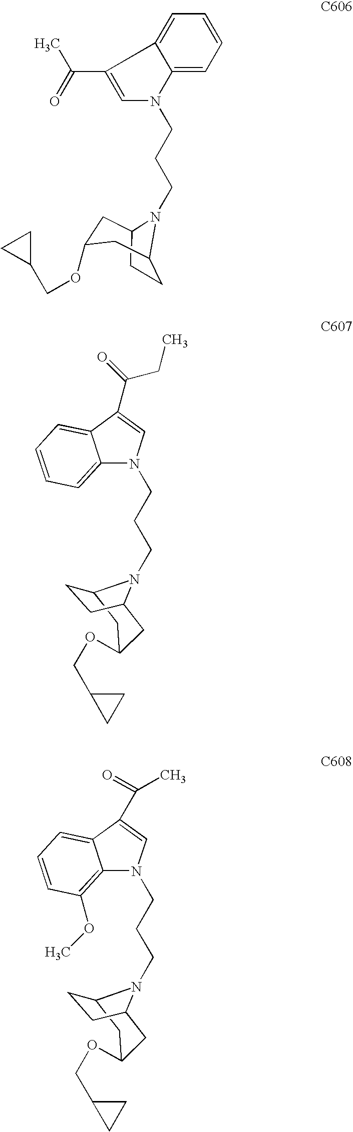

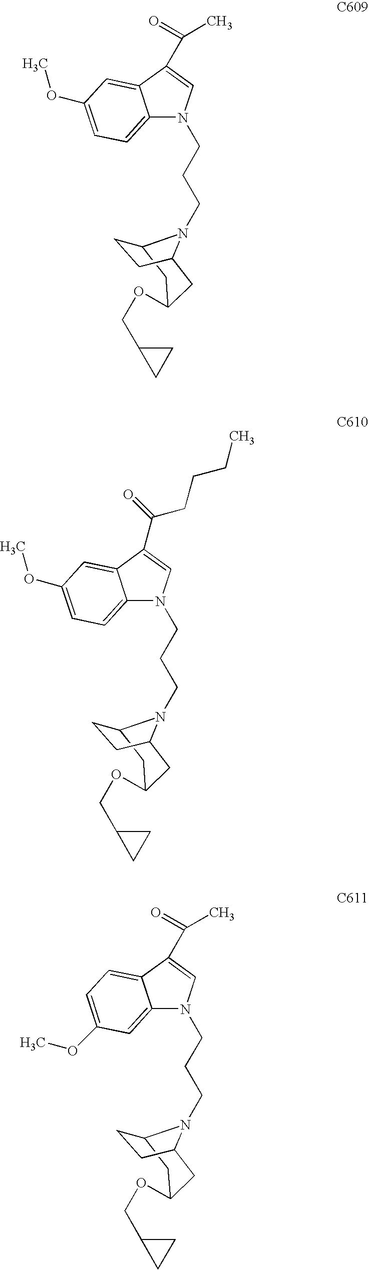

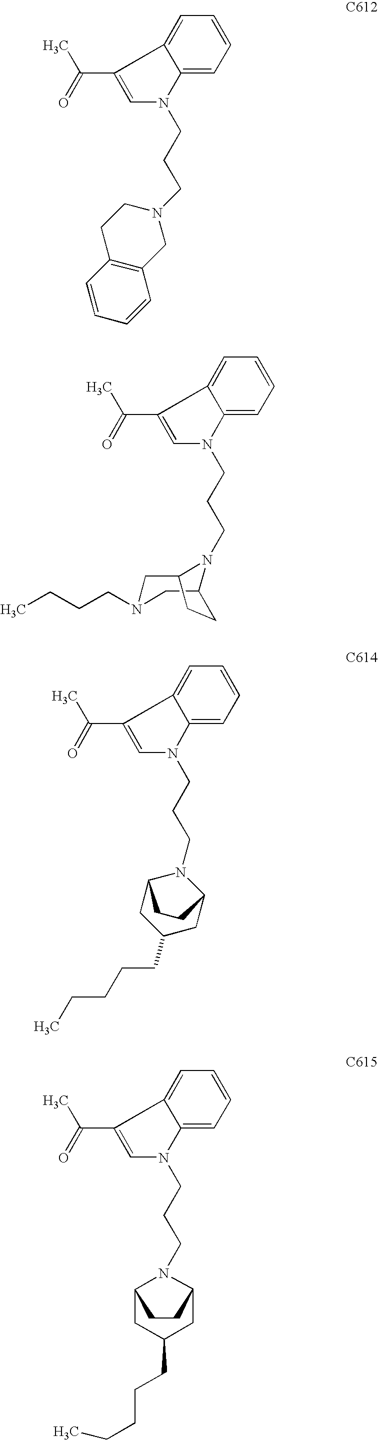

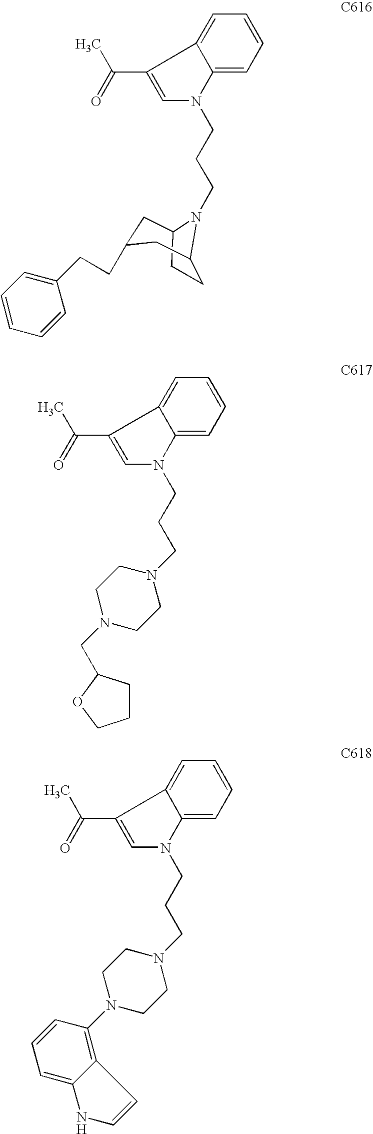

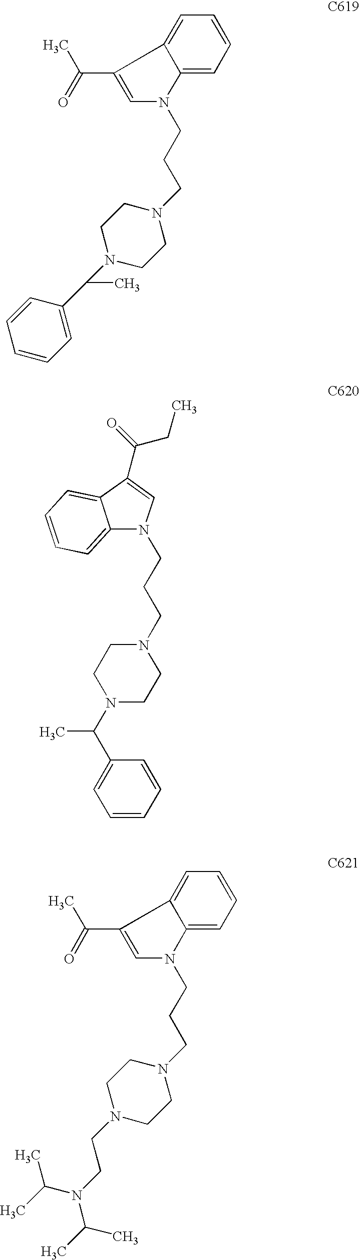

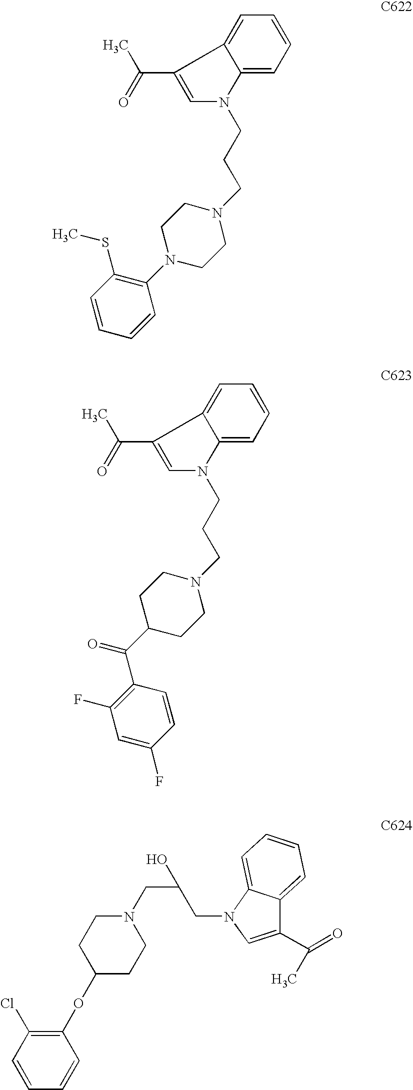

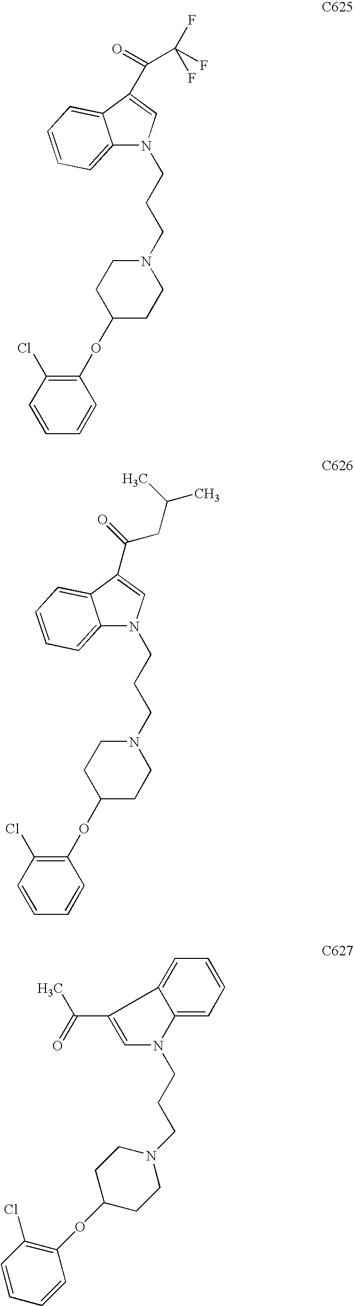

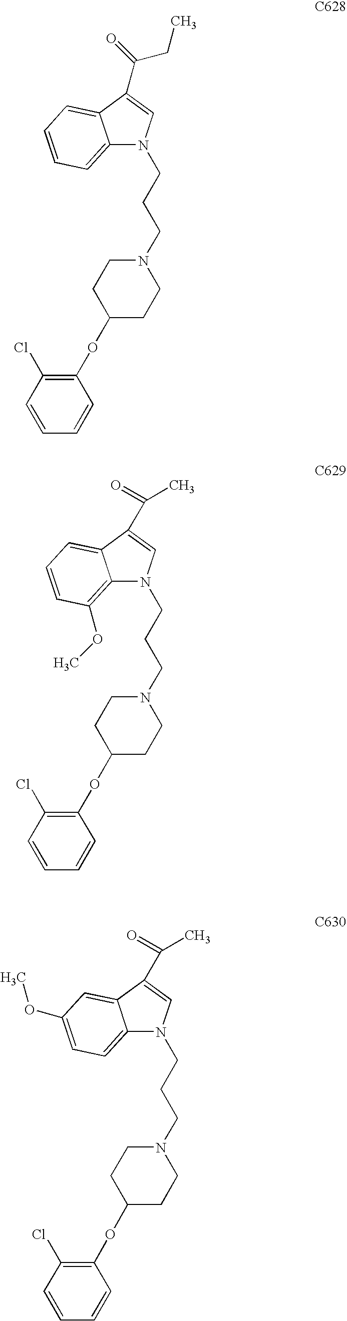

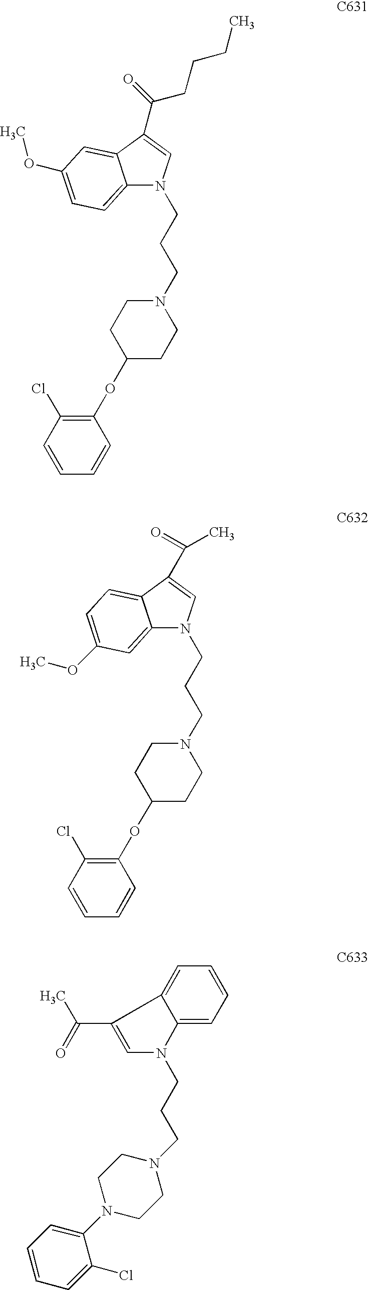

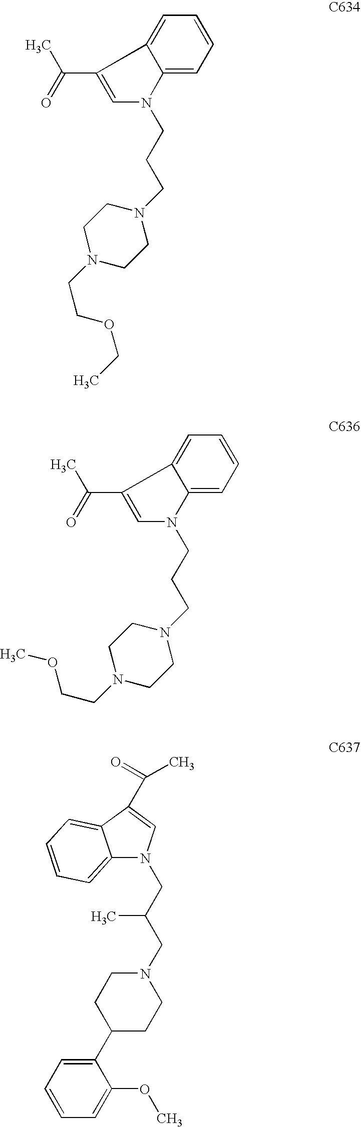

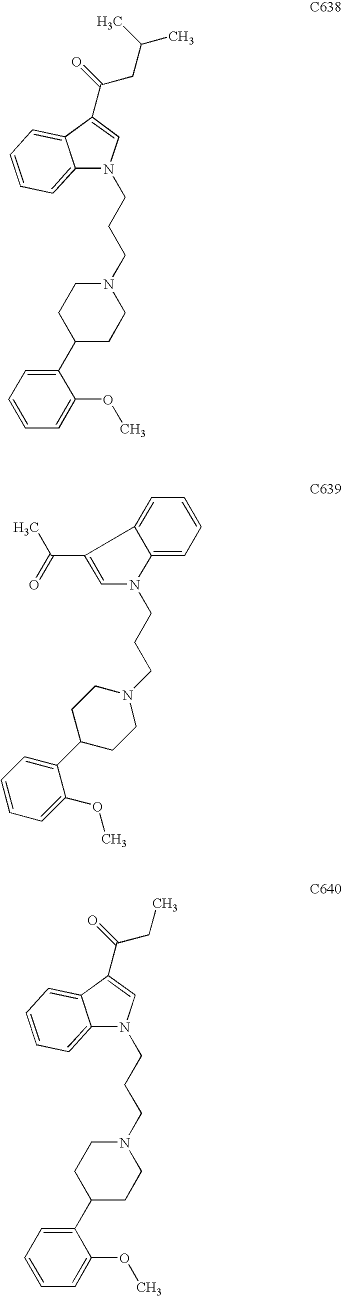

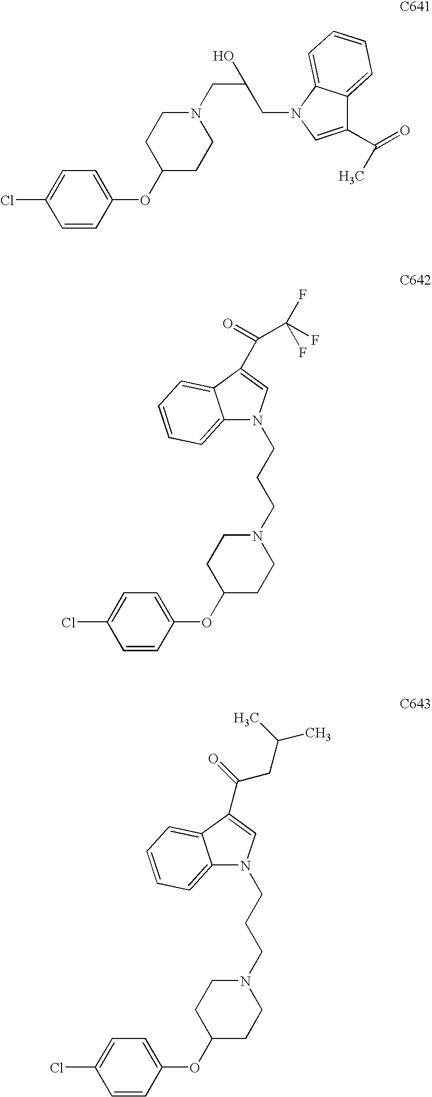

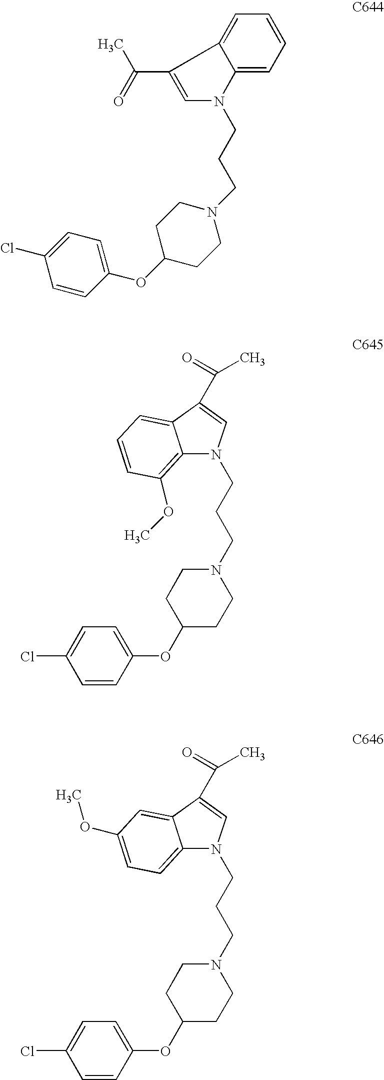

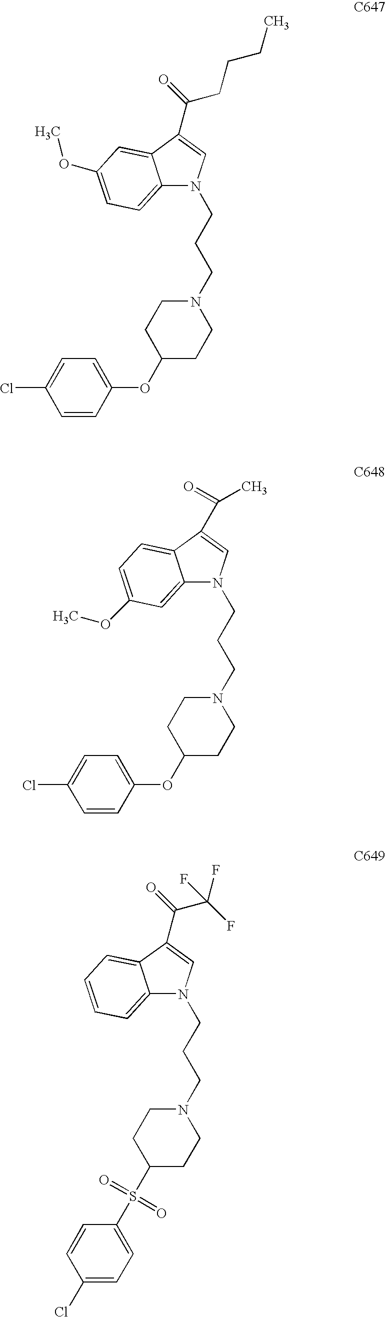

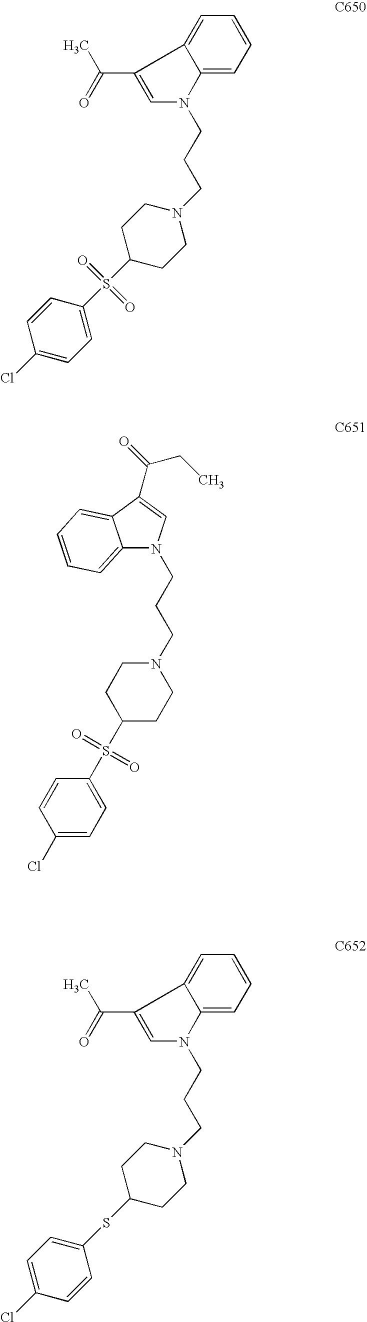

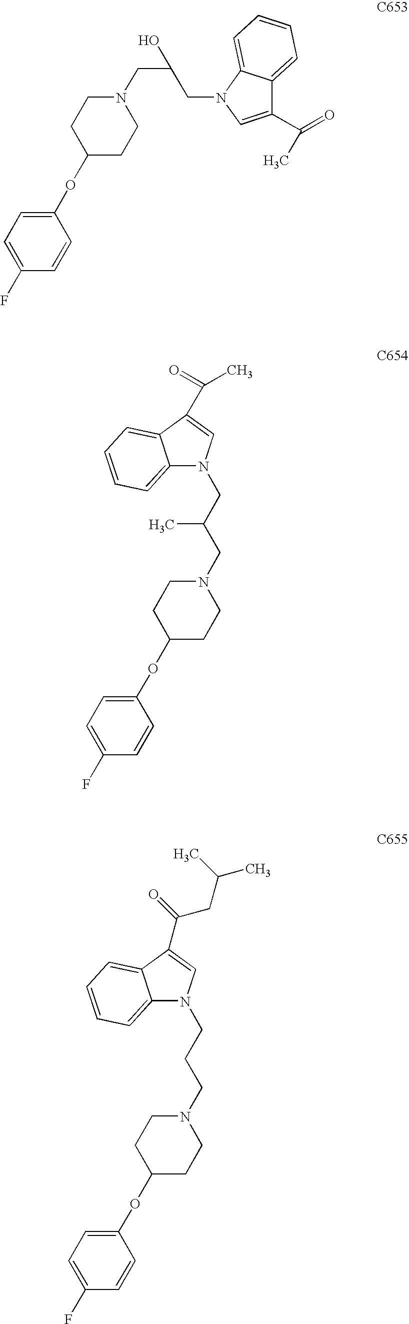

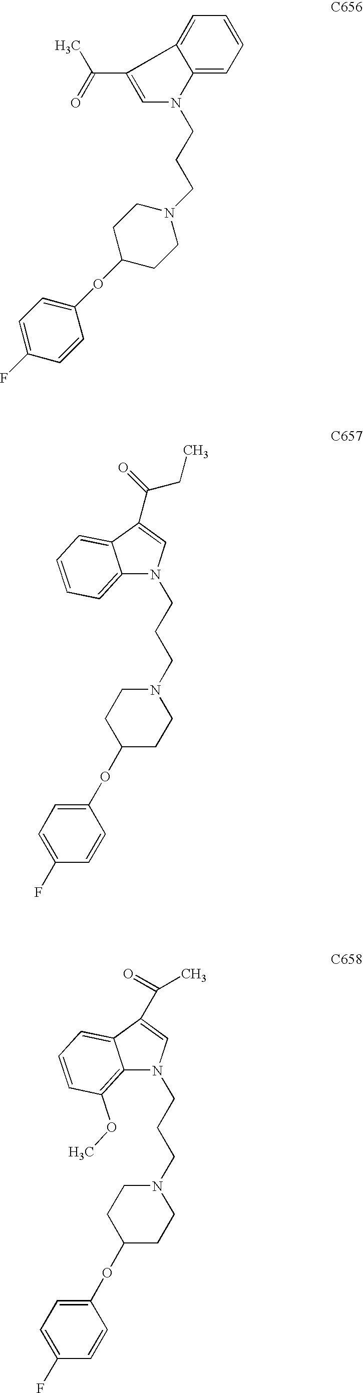

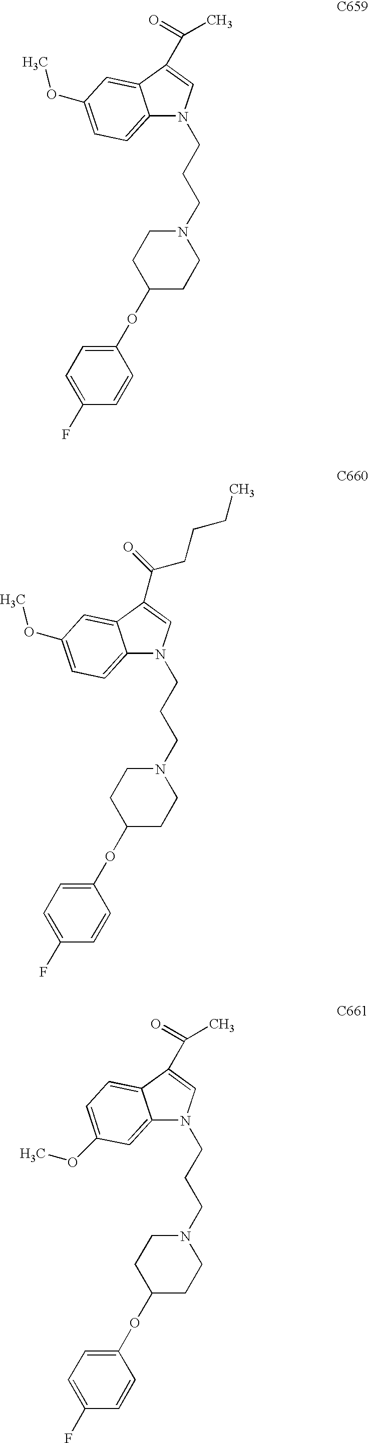

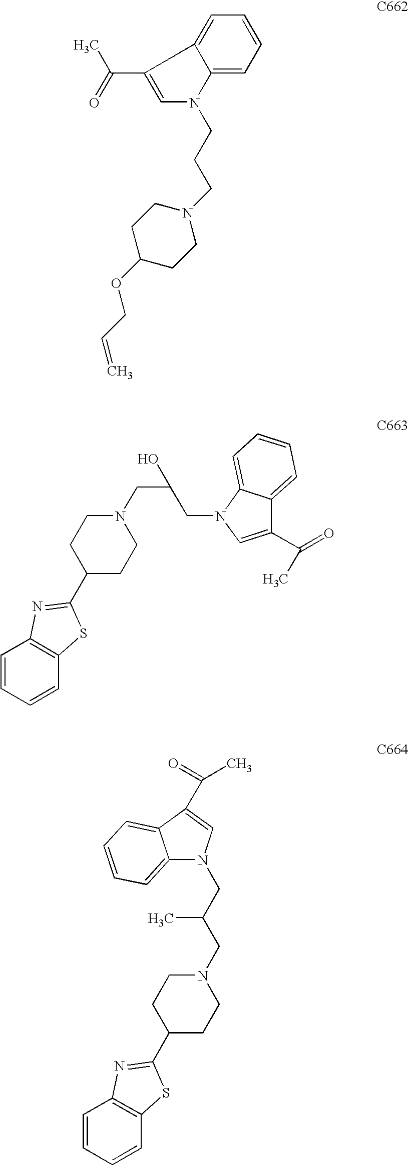

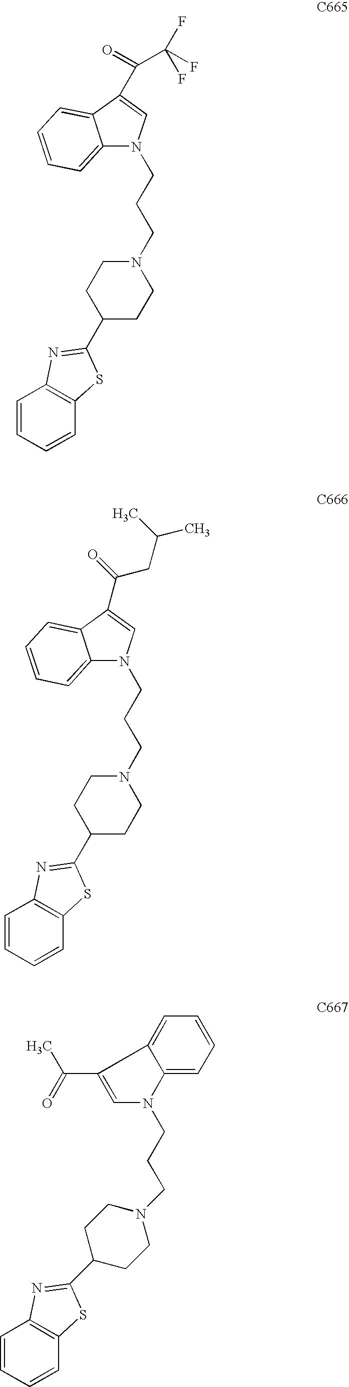

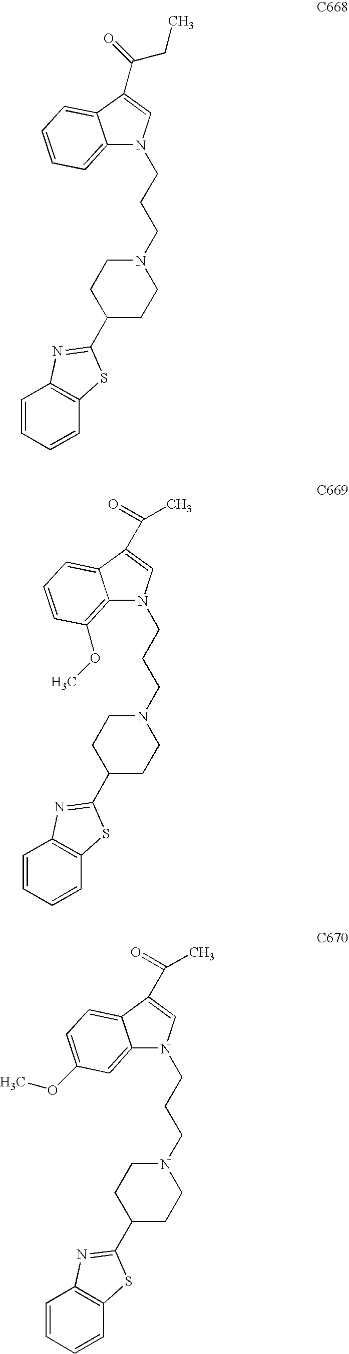

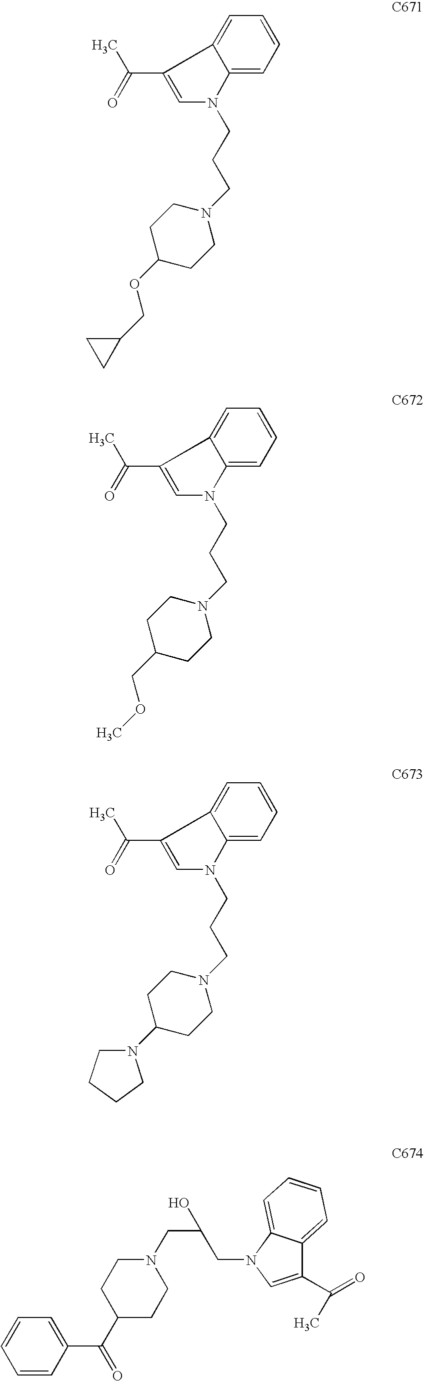

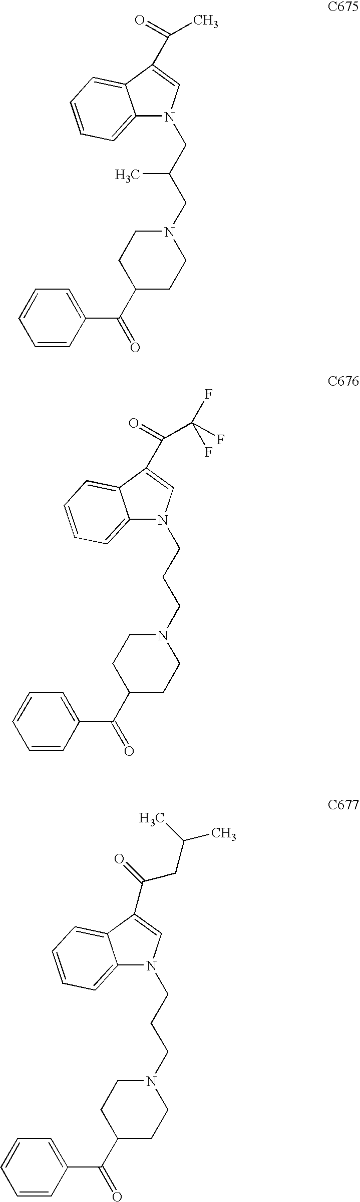

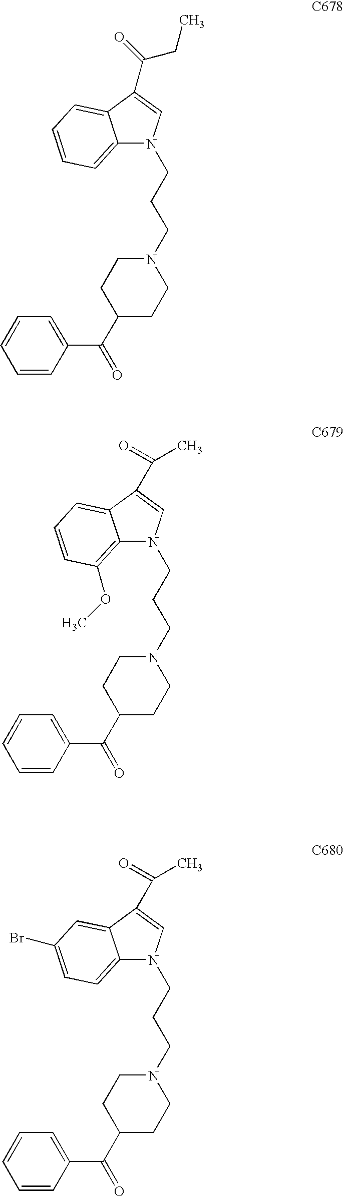

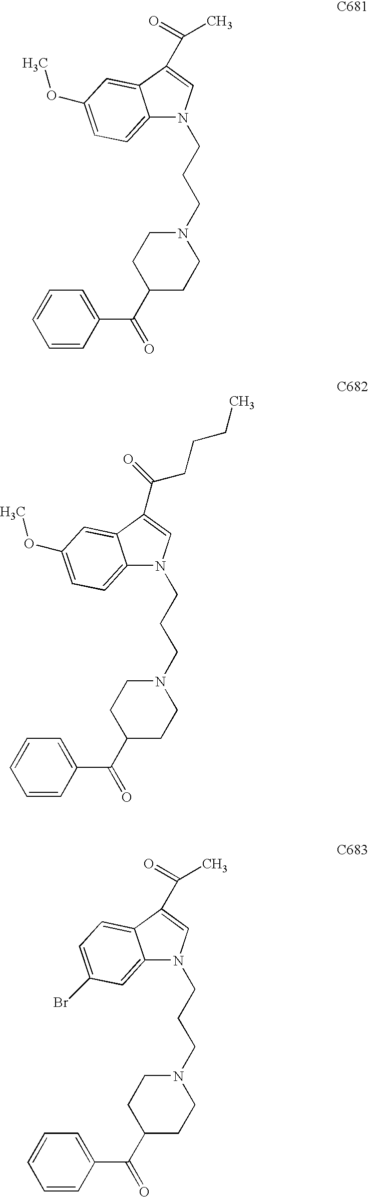

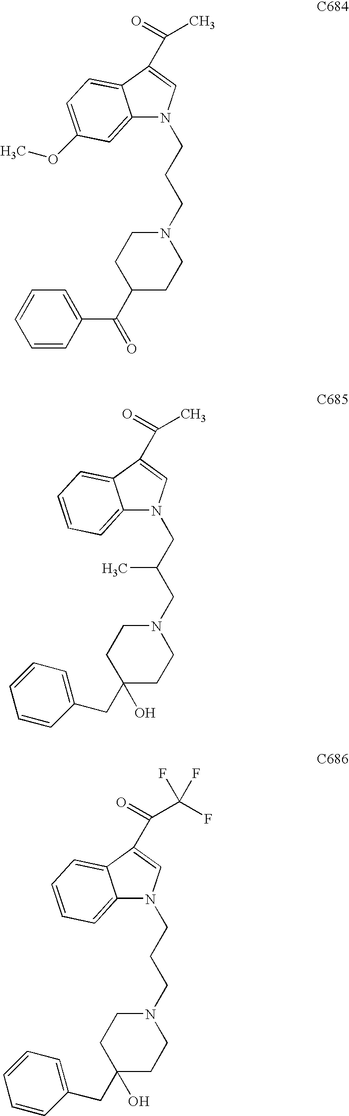

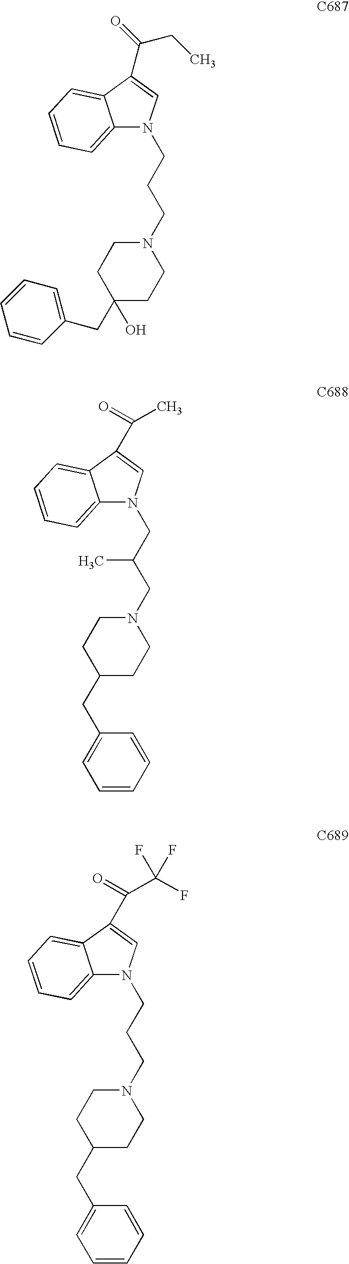

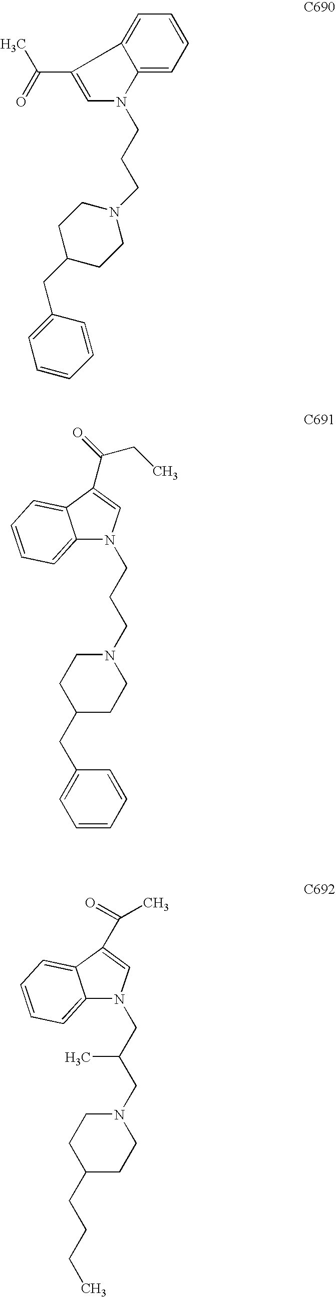

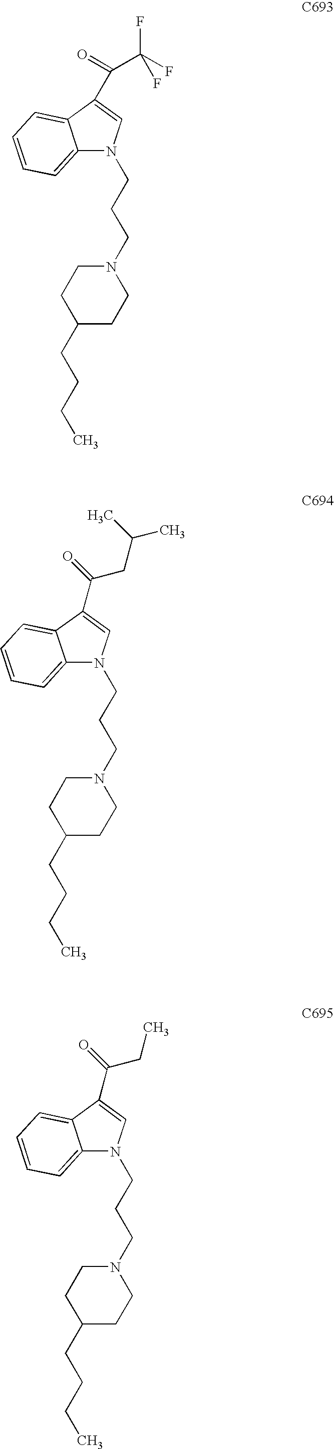

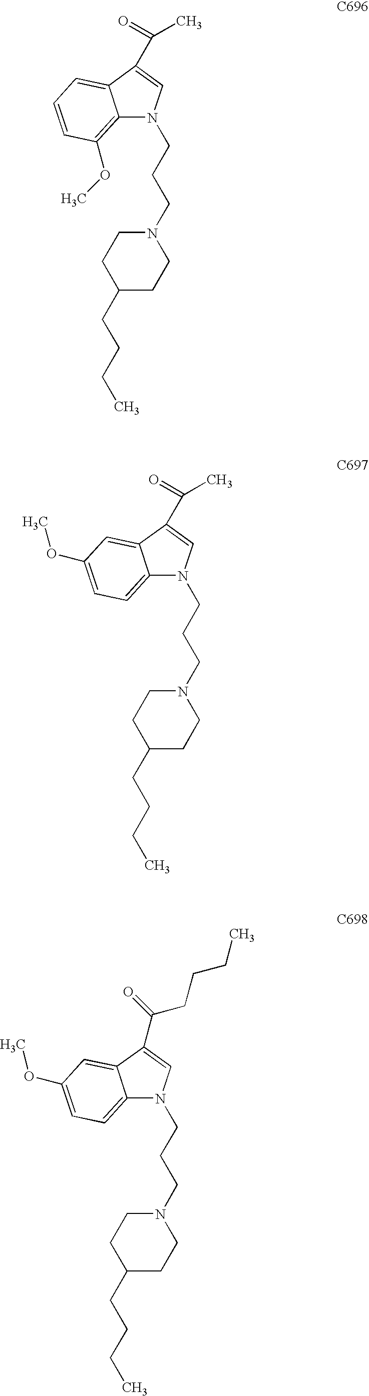

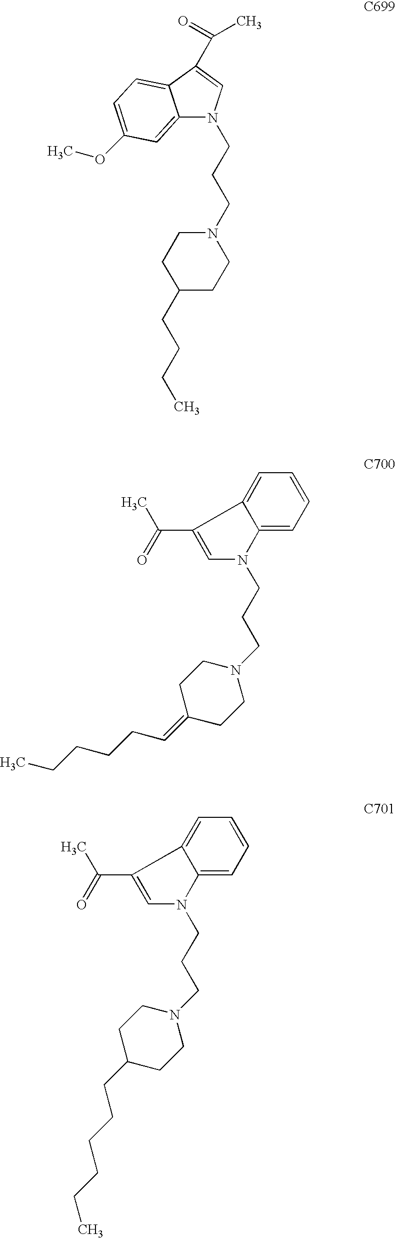

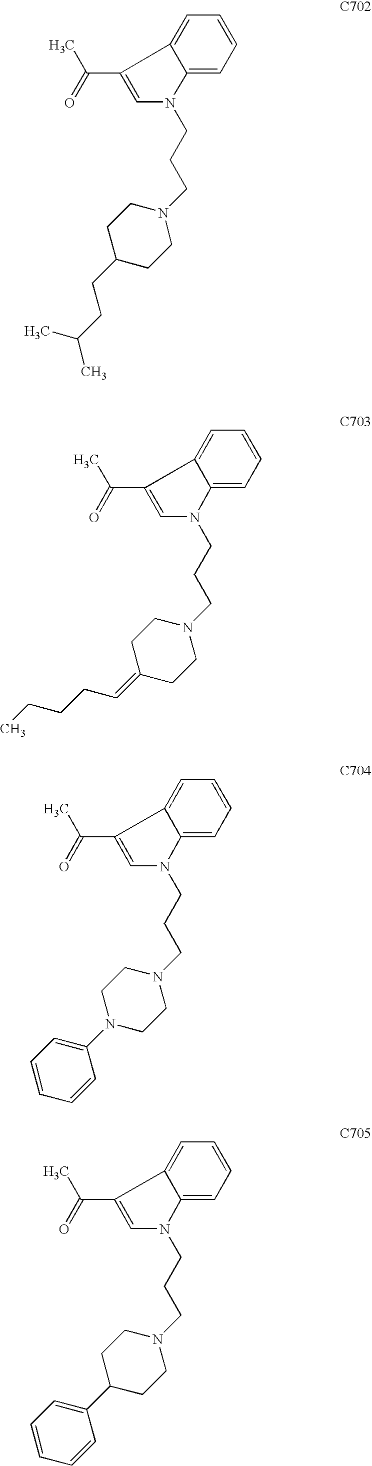

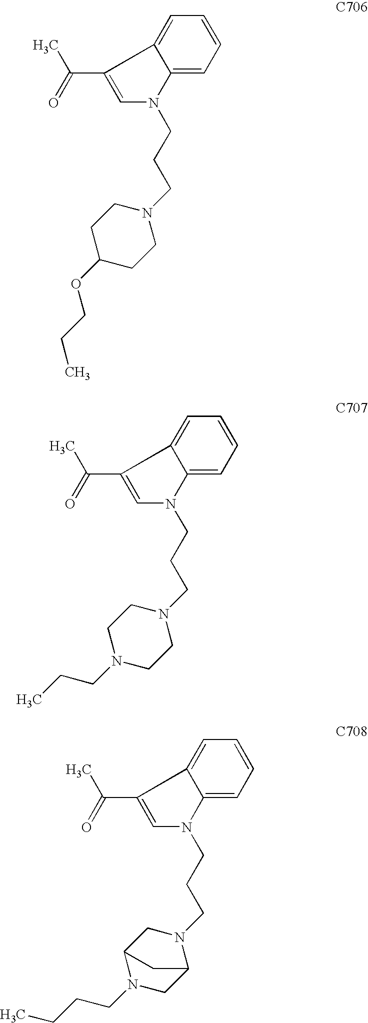

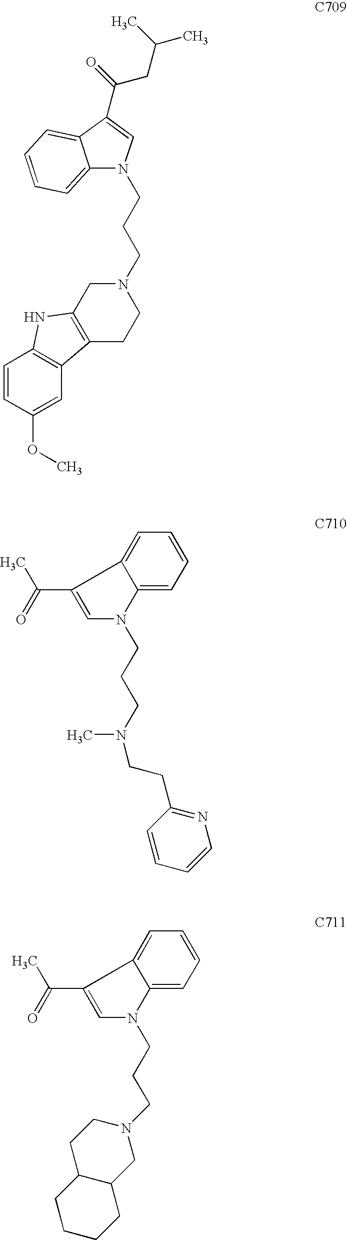

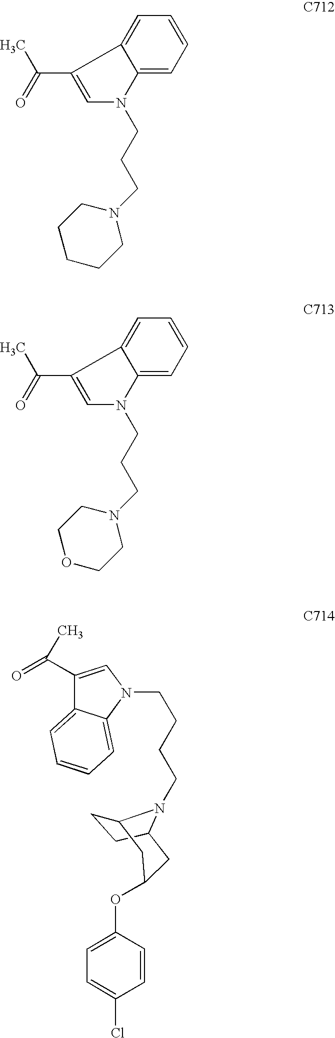

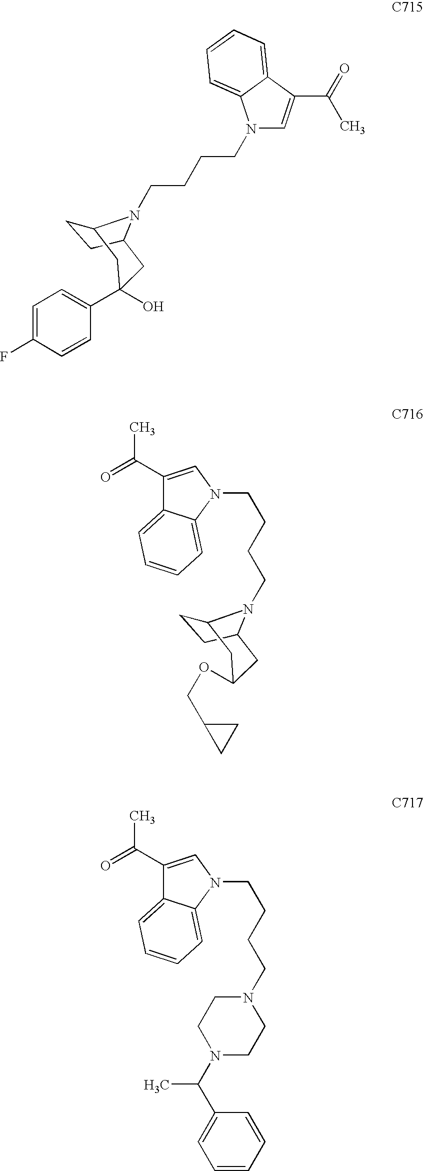

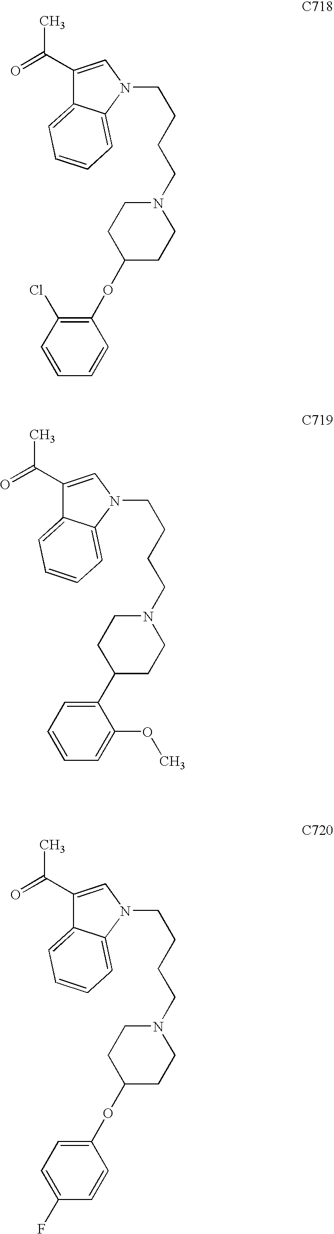

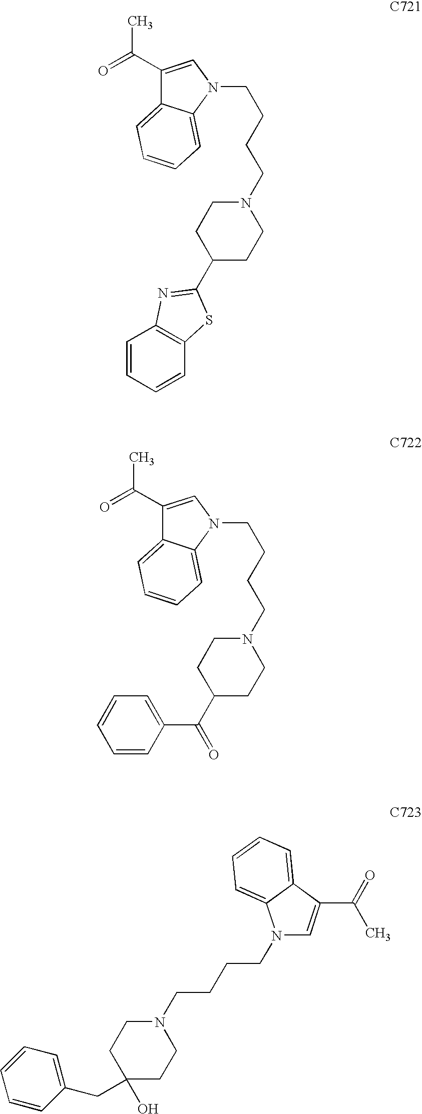

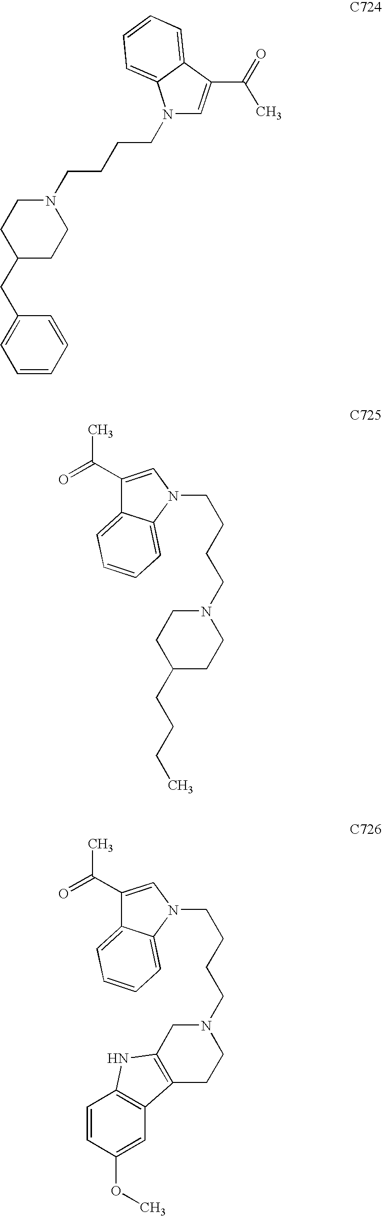

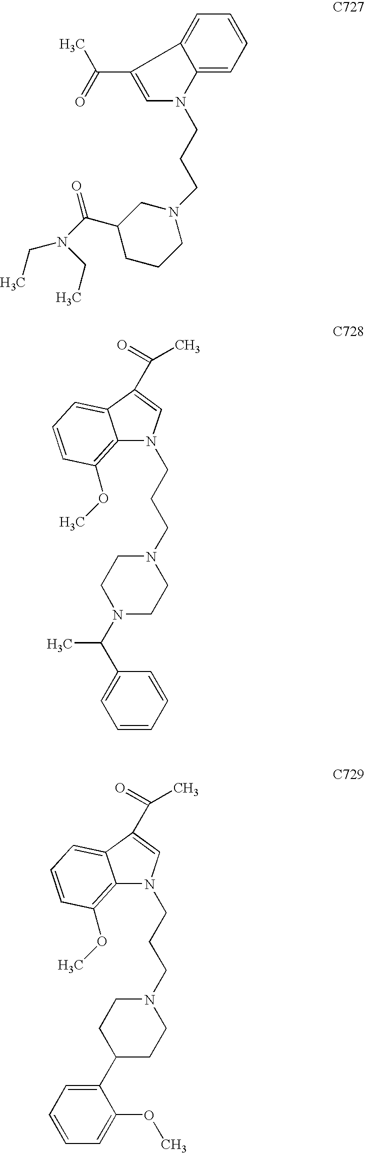

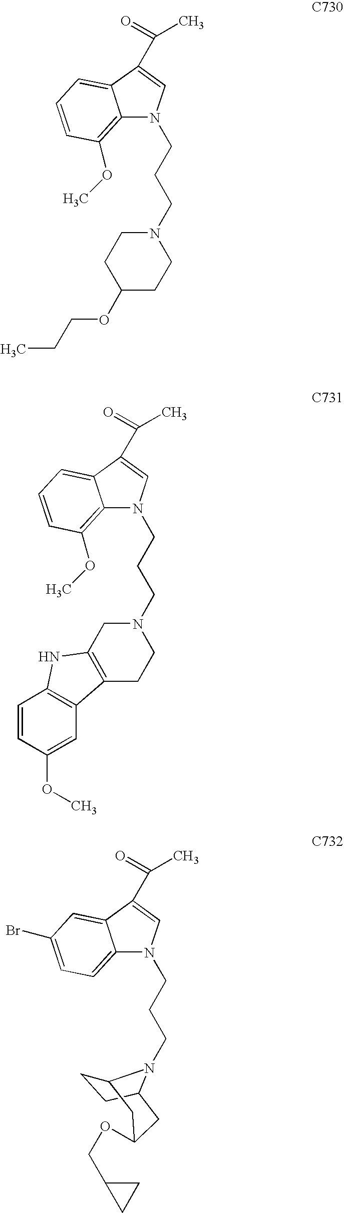

What is claimed is:

1. A compound of Formula (I): 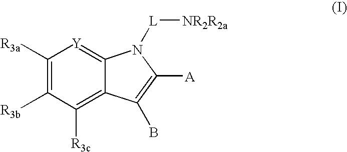
or a solvate, a polymorph, a metabolite, or a pharmaceutically acceptable salt or prodrug thereof, wherein:
A is selected from the group consisting of hydrogen, halogen, cyano, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(═O)R₁;
B is selected from the group consisting of hydrogen; mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, —C(=Z)R₁, —C(=Z)OR₁, —C(=Z)NR₁ₐR_{1b}, —C(=Z)N(OR₁ₐ)R_{1b}, —C(=Z)N(R₁)NR₁ₐR_{1b}, —C(R₁)═NR₁ₐ, —C≡N; —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —S(O)R₁, —S(O)₂R₁, —OR₁, —SR₁, and —OC(═O)R₁;
A and B can be taken together to form an unsubstituted or substituted cycloalkyl, or unsubstituted or substituted heteroalicyclyl;
R₁, R₁ₐ and R_{1b} are each independently selected from the group consisting of hydrogen, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl and haloalkyl;
R₂ and R₂ₐ are each independently selected from the group consisting of hydrogen, cyano, mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, sulfinyl, sulfonyl, haloalkyl, —C(=Z)R₁, —C(=Z)OR₁, —C(=Z)NR₁ₐR_{1b}, —C(R₁)═NR₁ₐ, —(C₁₋₄alkyl)-Z-aryl, —(C₁₋₄alkyl)C(═O) R₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁; or
R₂ and R₂ₐ can be taken together, along with the nitrogen atom to which they are attached, to form an unsubstituted or substituted heteroalicyclyl;
R₃, R₃ₐ, R_{3b}, and R_{3c} are each independently selected from the group consisting of hydrogen, halogen, cyano, nitro, mono-substituted, poly-substituted or unsubstituted variants of the following residues: alkyl, alkenyl, alkynyl, cycloalkyl, cycloalkenyl cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
R₃, R₃ₐ, R_{3b}, and R_{3c} can be taken together with one or more adjacent members of the group consisting of R₃, R₃ₐ, R_{3b}, and R_{3c} to form a cycloalkyl, cycloalkenyl, cycloalkynyl, or heteroalicyclyl ring;
R_{3c} can be taken together with B to form a cycloalkyl, cycloalkenyl, cycloalkynyl, or heteroalicyclyl ring;
L can be a unsubstituted or substituted lower alkylene group, wherein when L is substituted, it is substituted with one or more group(s) individually and independently selected from the group consisting of alkyl, alkenyl, halogen, haloalkyl, alkoxy, haloalkoxy, hydroxyl, and —CN;
L can be taken together with R₃ to form a cycloalkyl, cycloalkenyl, cycloalkynyl, or heteroalicyclyl ring;
Y is C—R₃ or N; and
Z is O or S.

2. The compound of claim 1, wherein the compound of Formula (I) modulates, agonizes, inverse agonizes, or antagonizes a ghrelin receptor.

3. The compound of claim 1, wherein the compound of Formula (I) inverse agonizes or antagonizes a ghrelin receptor.

4. The compound of claim 1, wherein Y is C—R₃.

5. The compound of claim 4, wherein R₃ is selected from the group consisting of hydrogen, halogen, cyano, nitro, mono-substituted, poly-substituted or unsubstituted variants of the following residues: alkyl, alkenyl, alkynyl, cycloalkyl, cycloalkenyl cycloalkynyl, aryl, heteroaryl, heteroalicyclyl, aralkyl, heteroaralkyl, (heteroalicyclyl)alkyl, sulfinyl, sulfonyl, haloalkyl, haloalkoxy, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁

6. The compound of claim 5, wherein R₃ is selected from the group consisting of alkyl, alkoxy, —C≡N, and halogen.

7. The compound of claim 6, wherein the alkyl is selected from the group consisting methyl, ethyl, n-propyl, isopropyl, n-butyl, isobutyl, and t-butyl.

8. The compound of claim 7, wherein the alkyl is selected from the group consisting methyl and ethyl.

9. The compound of claim 6, wherein the alkoxy is selected from the group consisting methoxy, ethoxy, n-propoxy, isopropoxy, n-butoxy, isobutoxy, and t-butoxy.

10. The compound of claim 9, wherein the alkoxy is methoxy.

11. The compound of claim 6, wherein R₃ is selected from the group consisting of alkyl, alkoxy, —C≡N, and halogen; and B is selected from the group consisting of —C(—O)R₁, —C(═O)OR₁, —C(=Z)NR₁ₐR_{1b}, —C(=Z)N(OR₁ₐ)R_{1b}, and —C≡N.

12. The compound of claim 1, wherein Y is N.

13. The compound of claim 1, wherein R₂ₐ is selected from the group consisting mono-substituted, poly-substituted or unsubstituted variants of the following residues: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, —(C₁₋₄alkyl)-Z-aryl, and —(C₁₋₄alkyl)C(═O)R₁.

14. The compound of claim 13, wherein the cycloalkenyl is

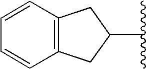

15. The compound of claim 13, wherein R₂ is hydrogen.

16. The compound of claim 13, wherein R₂ is an alkyl.

17. The compound of claim 1, wherein R₂ and R₂ₐ are taken together, along with the nitrogen atom to which they are attached, to form an unsubstituted or substituted heteroalicyclyl.

18. The compound of claim 1, wherein R₂ and R₂ₐ are taken together, along with the nitrogen atom to which they are attached, to form an unsubstituted or substituted heteroalicyclyl selected from the group consisting of: 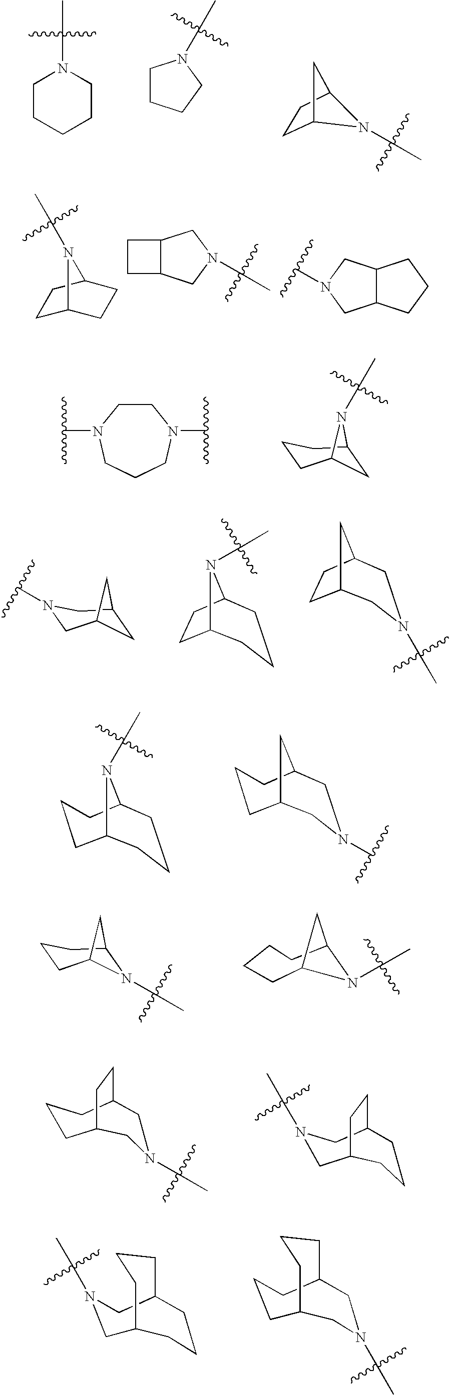 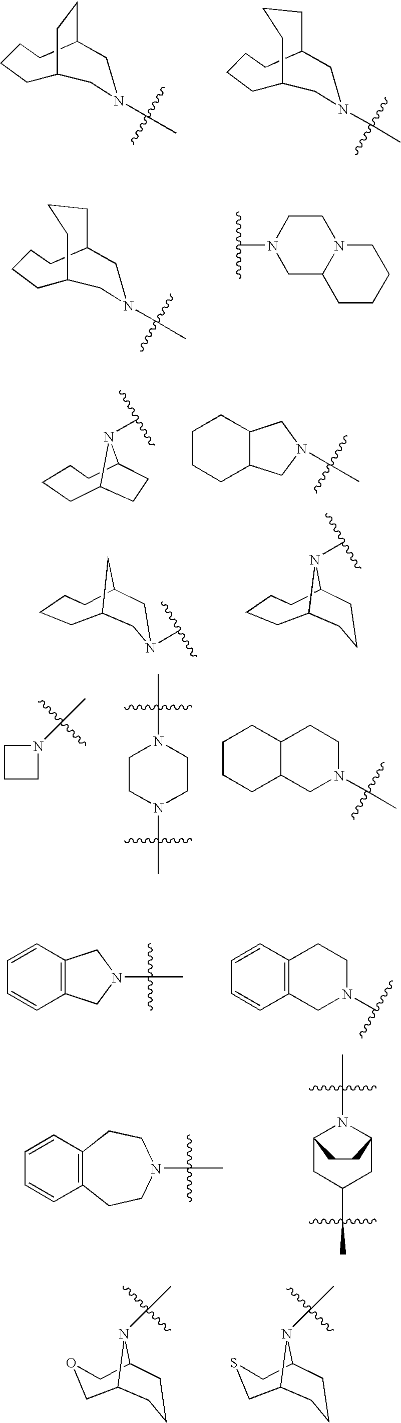 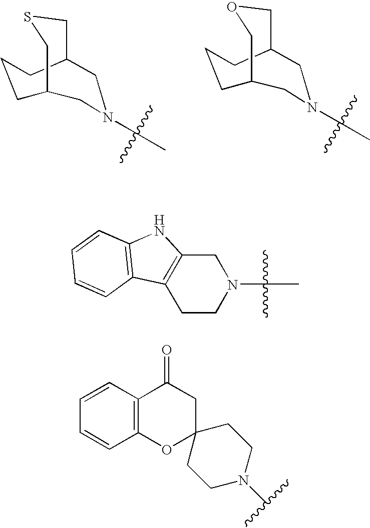
which is unsubstituted or substituted with one or more group(s) individually and independently selected from the group consisting of hydrogen, halogen, cyano, nitro, hydroxyl, mono-substituted, poly-substituted or unsubstituted variants of the following residues: alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, alkoxy, aryloxy, ester, mercapto, alkylthio, arylthio, carbonyl, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, C-carboxy, O-carboxy, silyl, sulfenyl, sulfinyl, sulfonyl, haloalkyl, haloalkoxy, trihalomethanesulfonyl, trihalomethanesulfonamido, and amino.

19. The compound of claim 18, wherein R₂ and R₂ₐ are taken together, along with the nitrogen atom to which they are attached, to form an unsubstituted or substituted heteroalicyclyl selected from the group consisting of: 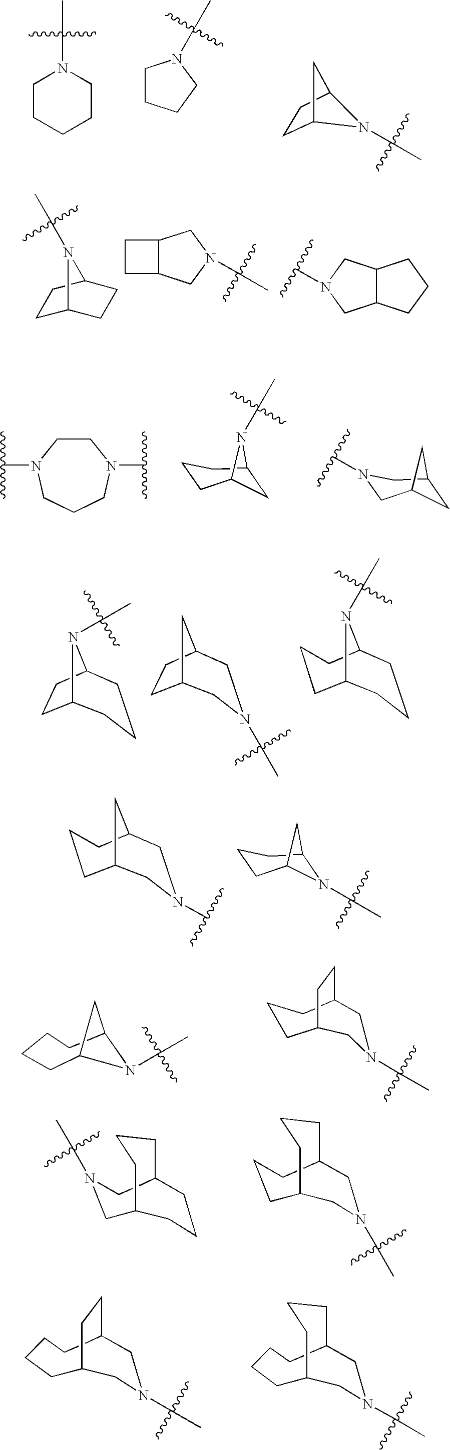 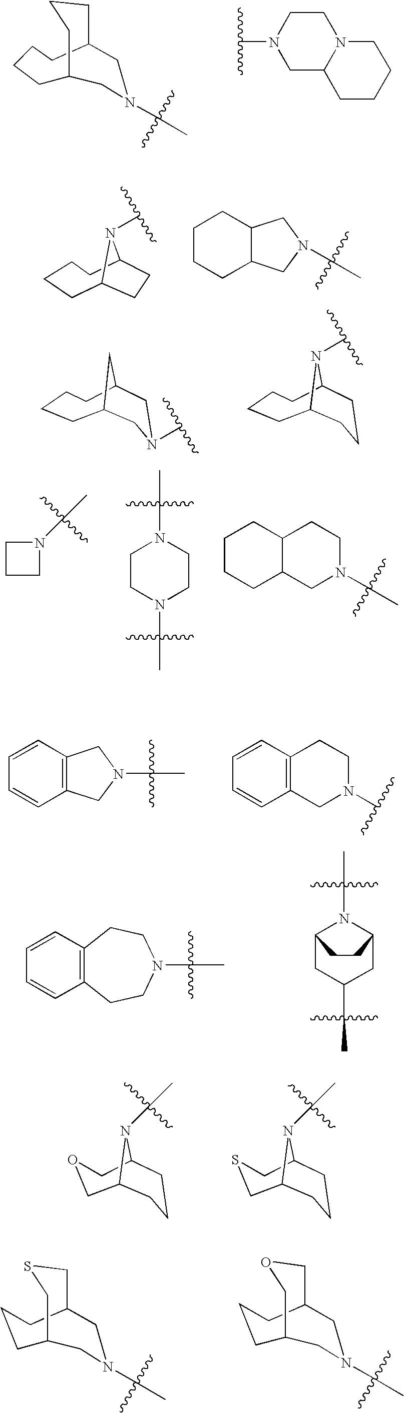 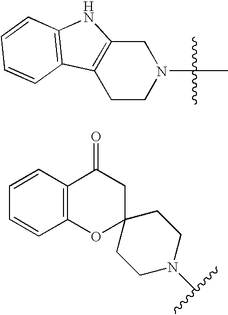
which is unsubstituted or substituted with one or more group(s) individually and independently selected from the group consisting of: 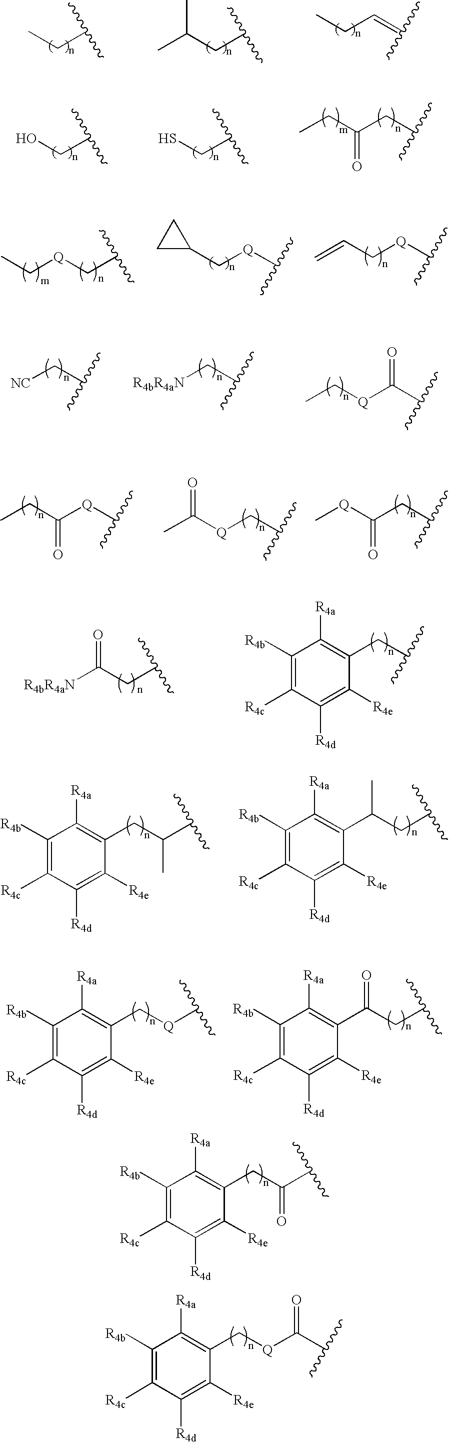 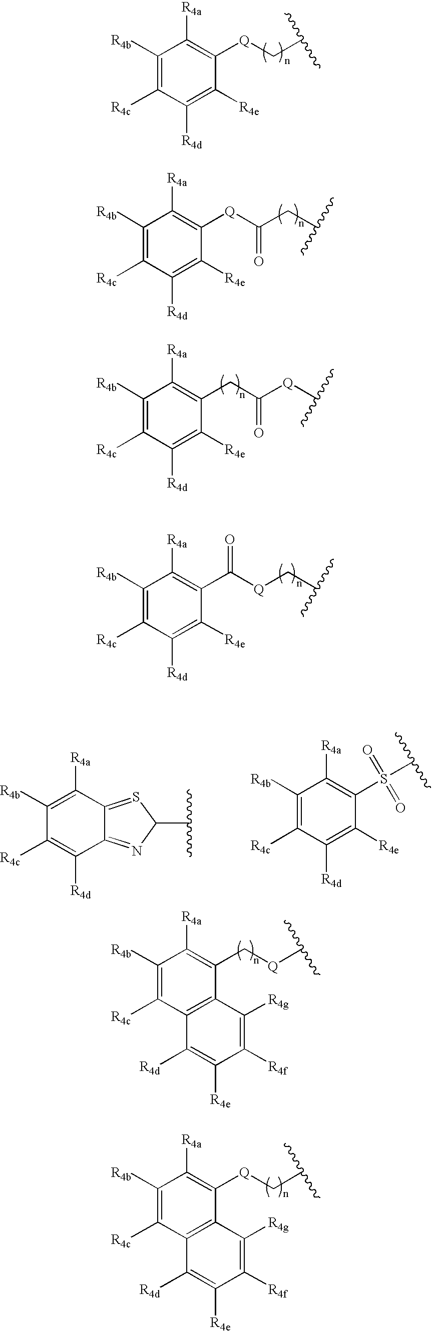 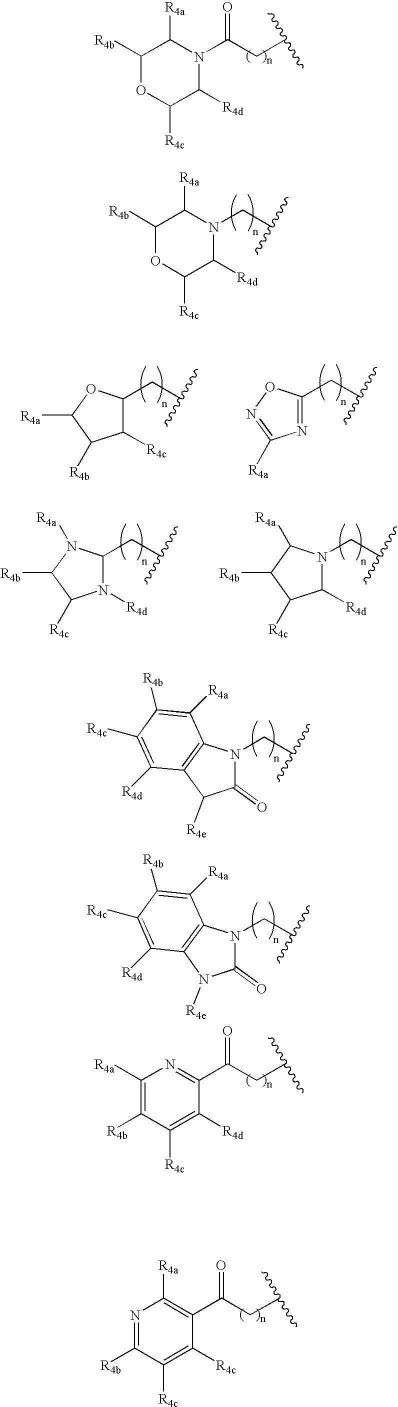 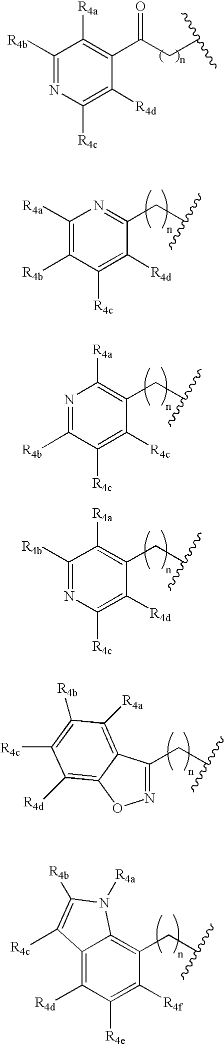
wherein:
n is integer selected from the group consisting of 0, 1, 2, 3, 4, 5, and 6;
m is integer selected from the group consisting of 0, 1, 2, 3, 4, 5, and 6;
Q is oxygen or sulfur; and
R₄ₐ, R_{4b}, R_{4c}, R_{4d}, R₄ₑ, and R_{f} are each independently selected from the group consisting of hydrogen, halogen, cyano, nitro, hydroxyl, mono-substituted, poly-substituted or unsubstituted variants of the following residues: alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, alkoxy, aryloxy, ester, mercapto, alkylthio, arylthio, carbonyl, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, C-carboxy, O-carboxy, silyl, sulfenyl, sulfinyl, sulfonyl, haloalkyl, haloalkoxy, trihalomethanesulfonyl, trihalomethanesulfonamido, and amino.

20. The compound of claim 19, wherein R₂ and R₂ₐ are taken together, along with the nitrogen atom to which they are attached, to form an unsubstituted or substituted heteroalicyclyl, which is unsubstituted or substituted with one or more group(s) individually and independently selected from the group consisting of:

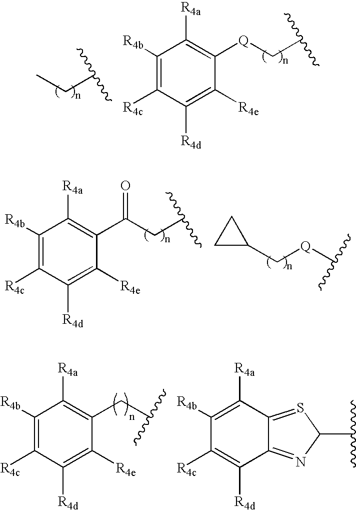

21. The compound of claim 19, wherein Q is oxygen.

22. The compound of claim 19, wherein Q is sulfur.

23. The compound of claim 19, wherein R₄ₐ, R_{4b}, R_{4c}, R_{4d}, and R₄ₑ are each independently selected from the group consisting of hydrogen, halogen, cyano, nitro, hydroxyl, mono-substituted, poly-substituted or unsubstituted variants of the following residues: alkyl, alkoxy, aryl, alkylthio, and haloalkyl.

24. The compound of claim 19, wherein at least one of R₄ₐ, R_{4b}, R_{4c}, R_{4d}, and R₄ₑ, is halogen.

25. The compound of claim 23, wherein the alkoxy is selected from the group consisting of methoxy, ethoxy, n-propoxy, isopropoxy, n-butoxy, isobutoxy, and t-butoxy.

26. The compound of claim 25, wherein the alkoxy is methoxy

27. The compound of claim 23, wherein the alkyl is selected from the group consisting of methyl, ethyl, n-propyl, isopropyl, n-butyl, isobutyl, and t-butyl.

28. The compound of claim 27, wherein the alkyl is selected from the group consisting of methyl and ethyl.

29. The compound of claim 23, wherein the aryl is pyridine.

30. The compound of claim 23, wherein the haloalkyl is CF₃.

31. The compound of claim 19, wherein n is an integer selected from the group consisting of 0, 1, 2, and 3.

32. The compound of claim 19, wherein m is an integer selected from the group consisting of 1, 2, and 3.

33. The compound of claim 19, wherein 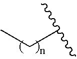
is n-butyl or n-pentyl.

34. The compound of claim 18, wherein R₂ and R₂ₐ are taken together, along with the nitrogen atom to which they are attached, to form an unsubstituted or substituted heteroalicyclyl selected from the group consisting of:

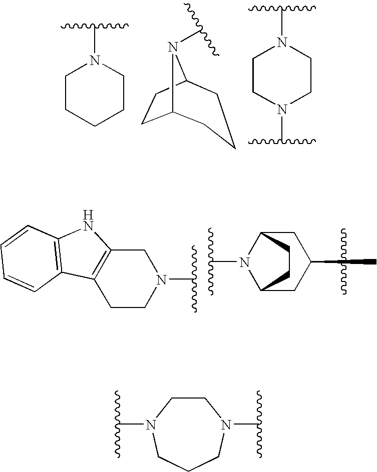

35. The compound of claim 34, wherein R₂ and R₂ₐ are taken together, along with the nitrogen atom to which they are attached, to form an unsubstituted or substituted heteroalicyclyl selected from the group consisting of:

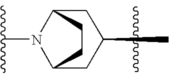

36. The compound of claim 1, wherein B is selected form the group consisting of —C(—O)R₁, —C(—O)OR₁, —C(=Z)NR₁ₐR_{1b}, —C(=Z)N(OR₁ₐ)R_{1b}, and —C≡N

37. The compound of claim 36, wherein B is —C(═O)R₁.

38. The compound of claim 36, wherein B is —C(=Z)NR₁ₐR_{1b}.

39. The compound of claim 1, wherein R₁, R₁ₐ and R_{1b} are each independently selected from the group consisting of: hydrogen, mono-substituted, poly-substituted or unsubstituted variants of the following residues: alkyl, alkenyl, cycloalkyl, aryl, aralkyl, and haloalkyl.

40. The compound of claim 39, wherein said alkyl is selected from the group consisting of methyl, ethyl, n-propyl, isopropyl, n-butyl, isobutyl, t-butyl, linear or branched pentyl, linear or branched hexyl, linear or branched heptyl, and linear or branched octyl.

41. The compound of claim 40, wherein said alkyl is selected from the group consisting of methyl, ethyl, n-butyl, isobutyl, linear hexyl, and branched octyl.

42. The compound of claim 39, wherein said aryl is phenyl.

43. The compound of claim 39, wherein said cycloalkyl is cyclopropyl.

44. The compound of claim 39, wherein said haloalkyl is CF₃.

45. The compound of claim 39, wherein said aralkyl is optionally substituted phenyl(C₁₋₄alkyl).

46. The compound of claim 45, wherein said aralkyl is optionally substituted phenyl(methyl)

47. The compound of claim 45, wherein said optionally substituted phenyl(C₁₋₄alkyl) is substituted with a substituent selected from the group consisting of alkyl and halogen.

48. The compound of claim 47, wherein the optionally substituted phenyl(C₁₋₄alkyl) is substituted is methyl.

49. The compound of claim 1, wherein R₃, R₃ₐ, R_{3b}, and R_{3c} are each independently selected from the group consisting of hydrogen, halogen; mono-substituted, poly-substituted or unsubstituted variants of the following residues: alkyl and —OR₁.

50. The compound of claim 49, wherein said alkyl is selected from the group consisting of methyl, ethyl, n-propyl, isopropyl, n-butyl, isobutyl, and t-butyl.

51. The compound of claim 49, wherein the alkyl is selected from the group consisting of methyl and ethyl.

52. The compound of claim 49, wherein R₁ is selected from the group consisting of methyl, ethyl, n-propyl, isopropyl, n-butyl, isobutyl, and t-butyl.

53. The compound of claim 52, wherein R₁, is selected from the group consisting of methyl and isopropyl.

54. The compound of claim 1, wherein A is hydrogen.

55. The compound of claim 1, wherein A is alkyl.

56. The compound of claim 55, wherein the alkyl is methyl.

57. The compound of claim 1, wherein L is an unsubstituted or substituted lower alkylene group.

58. The compound of claim 57, wherein the lower alkylene group is ethylene, propylene, or butylene.

59. The compound of claim 1, wherein the compound of Formula (I) is selected from the group consisting of:

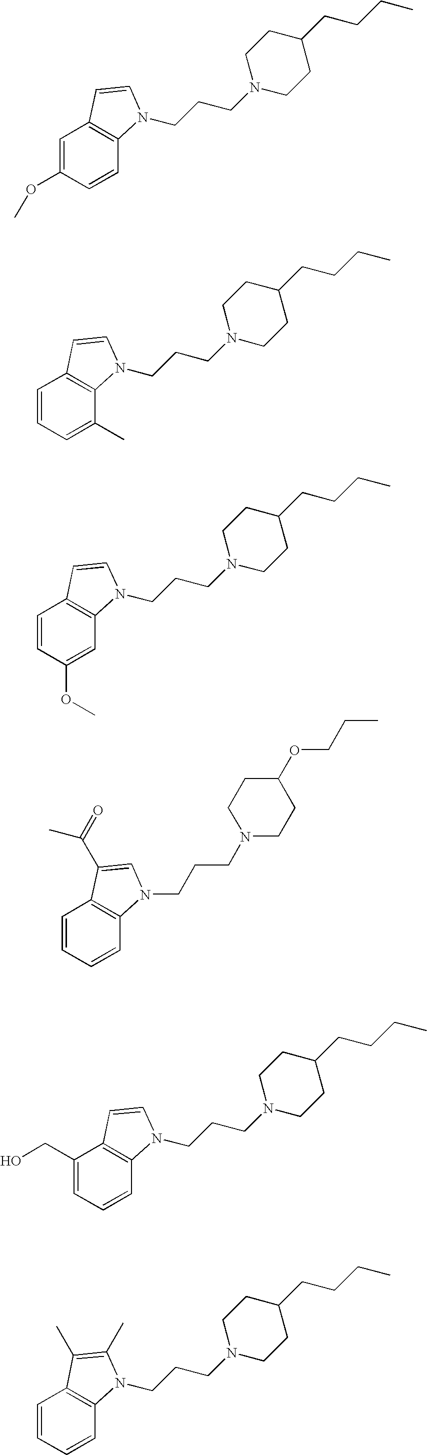

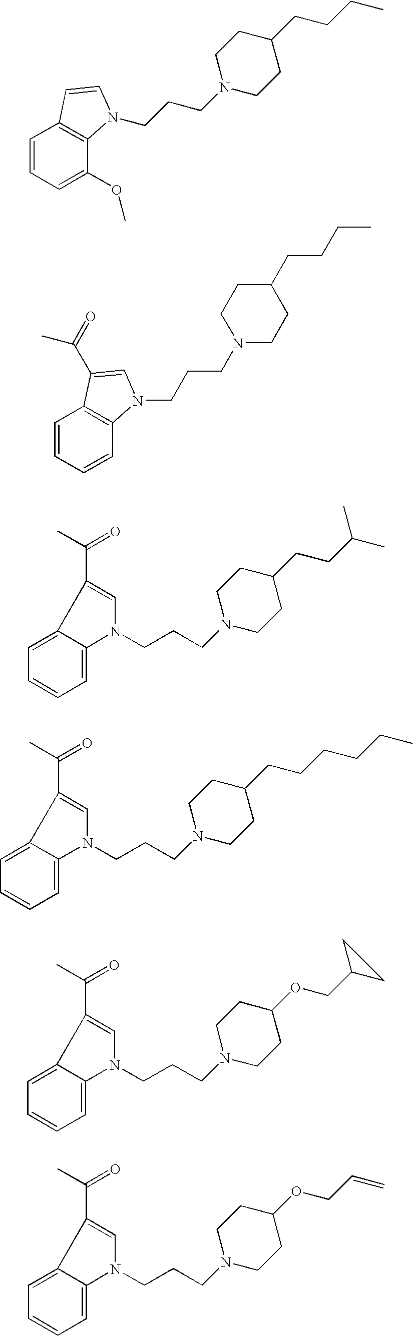

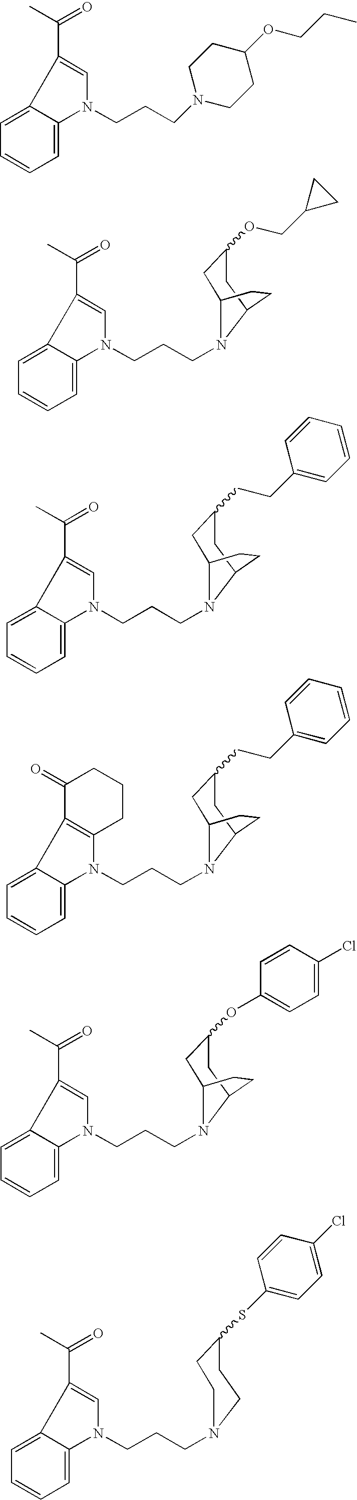

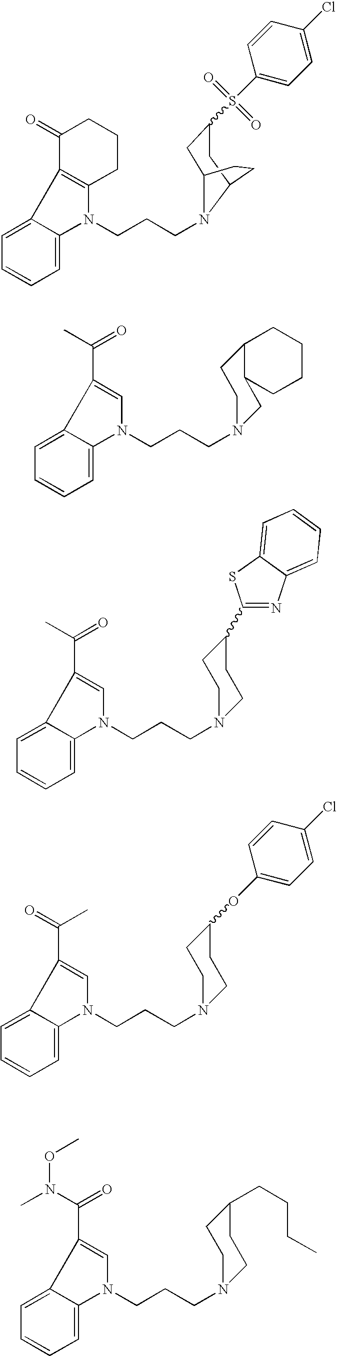

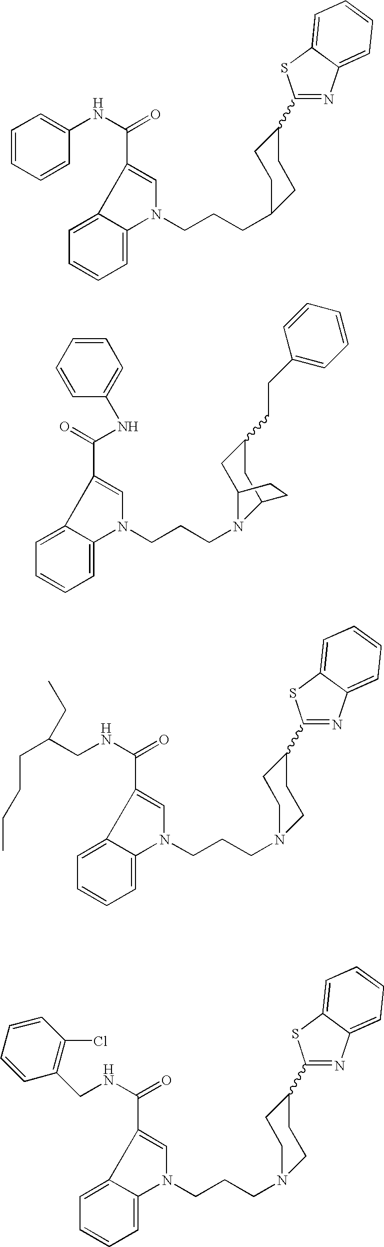

60. The compound of claim 1, wherein the compound is selected from the group consisting of:

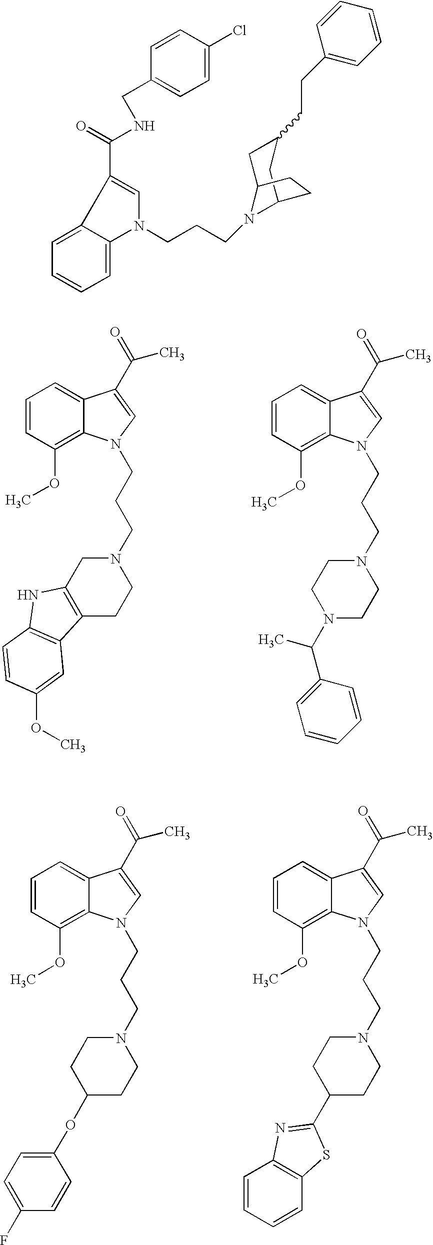

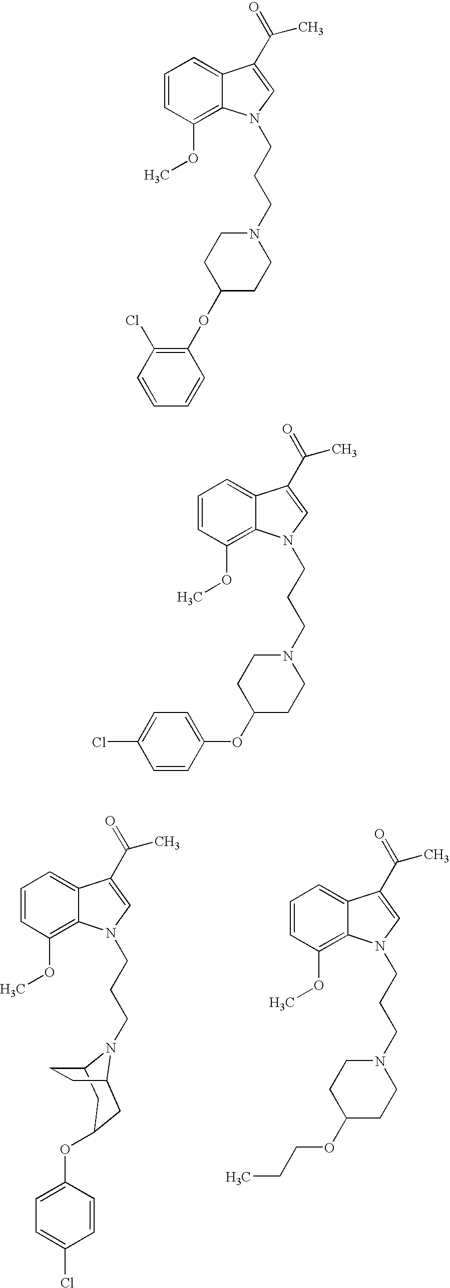

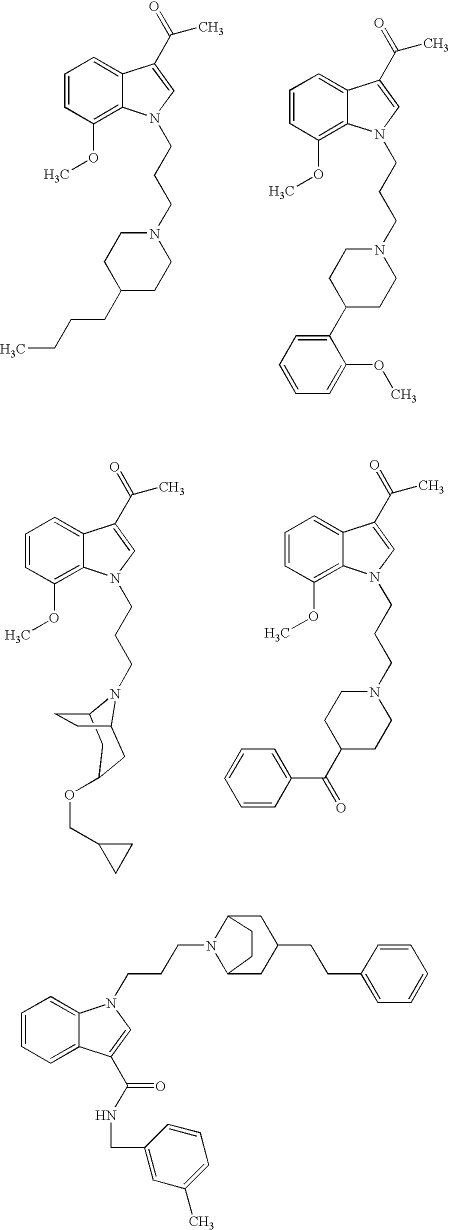

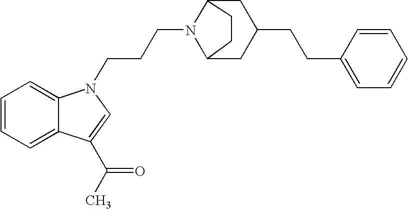

61. The compound of claim 1, wherein the compound is selected from the group consisting of:

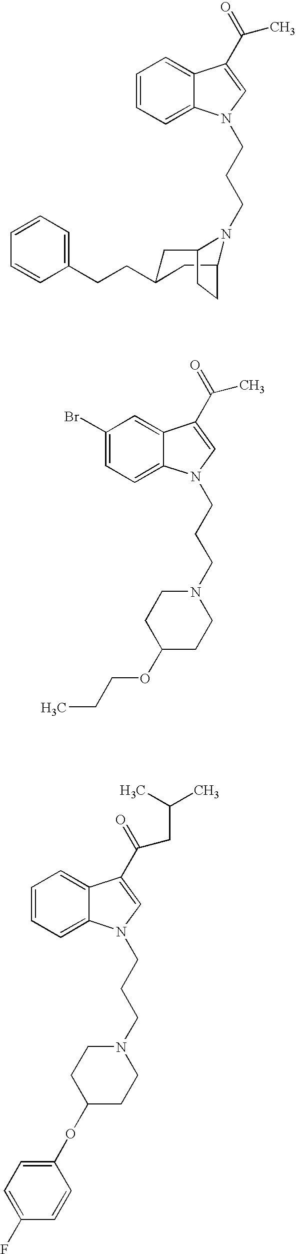

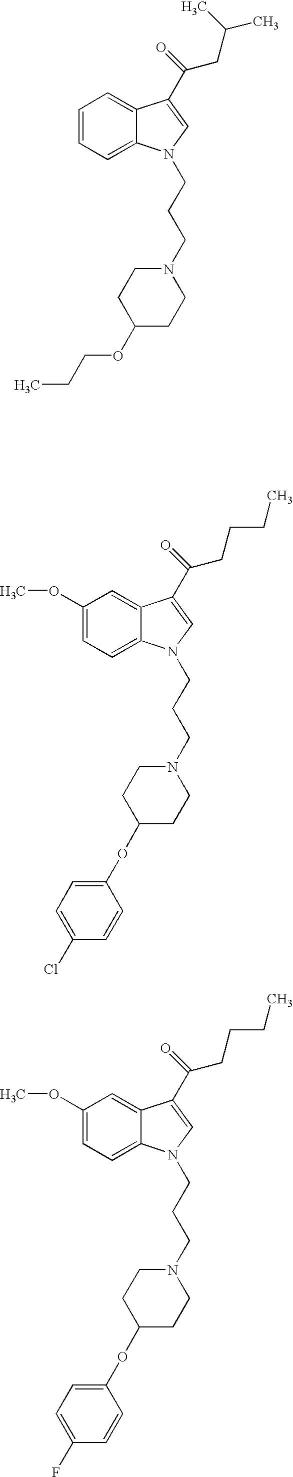

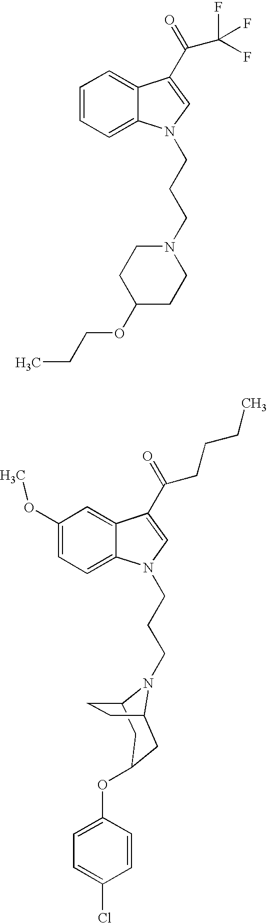

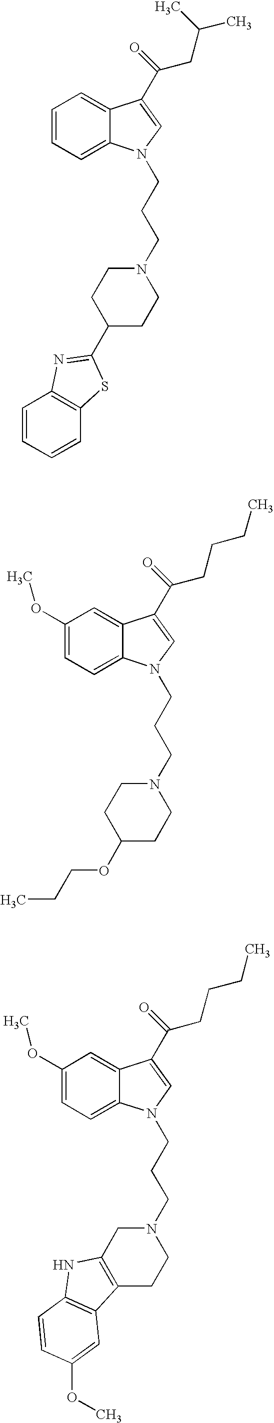

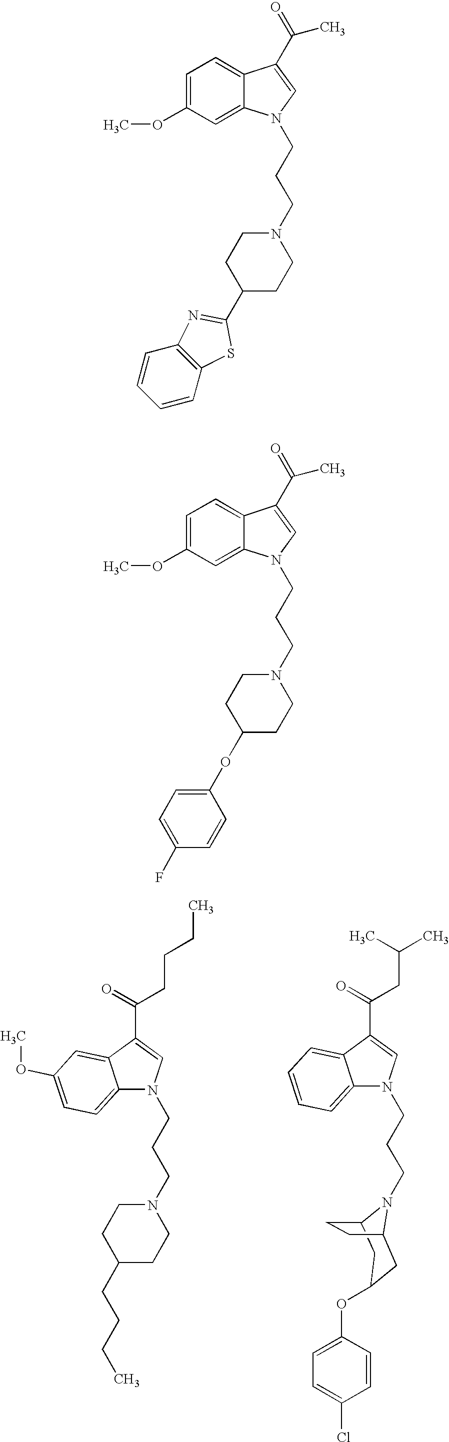

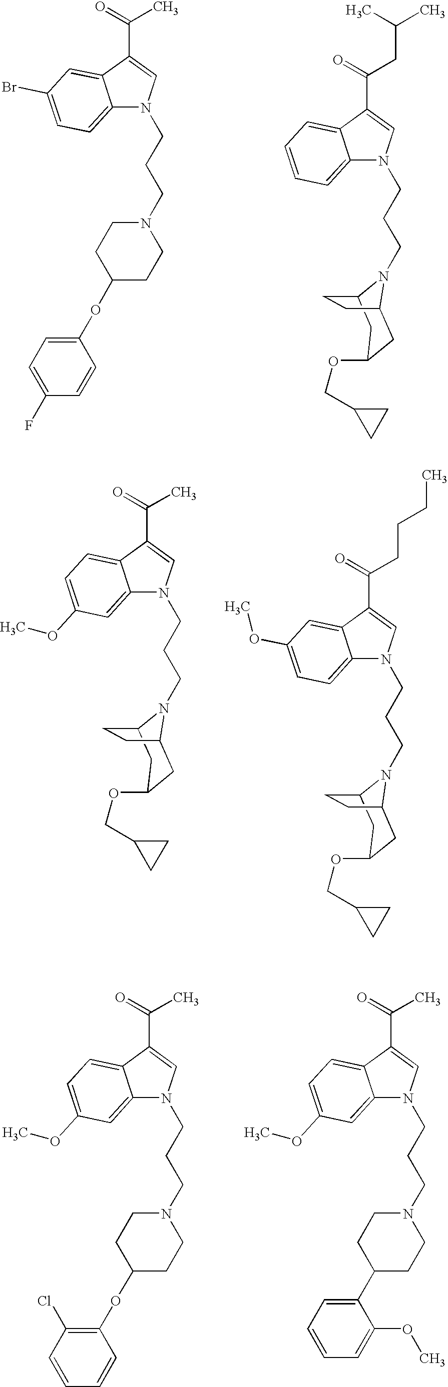

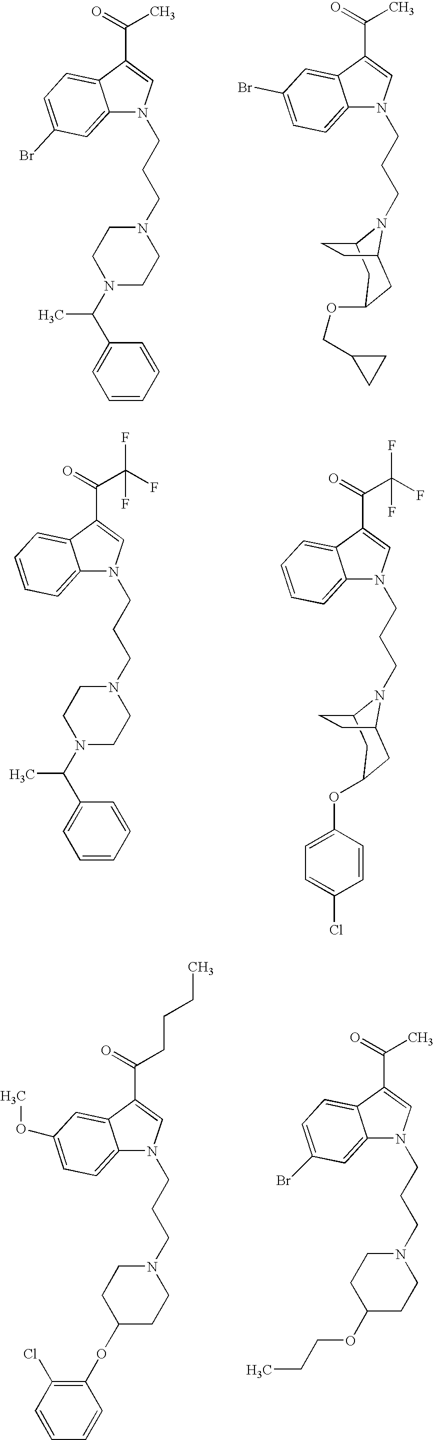

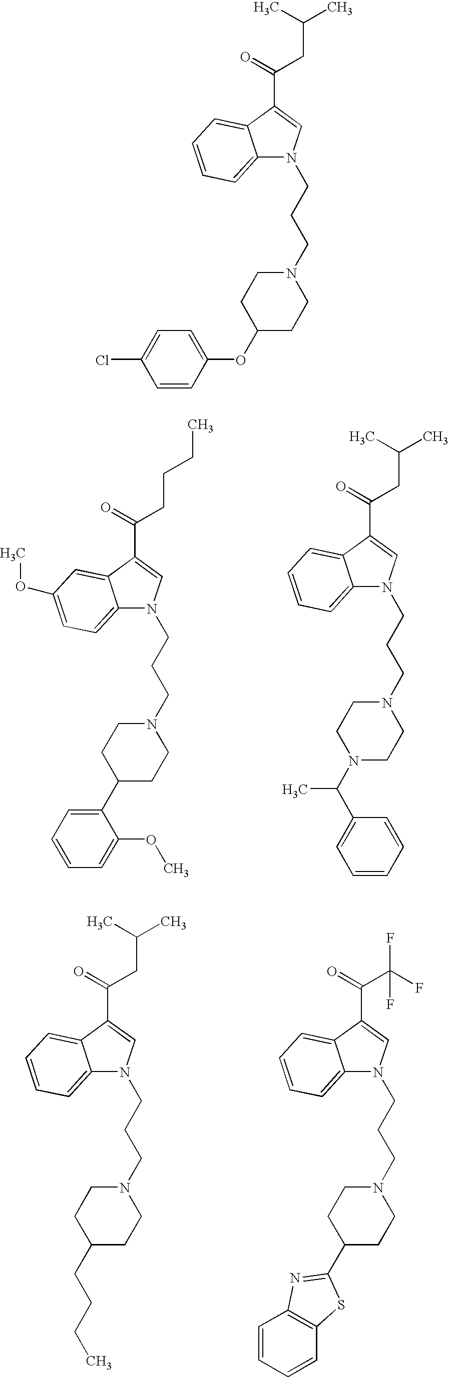

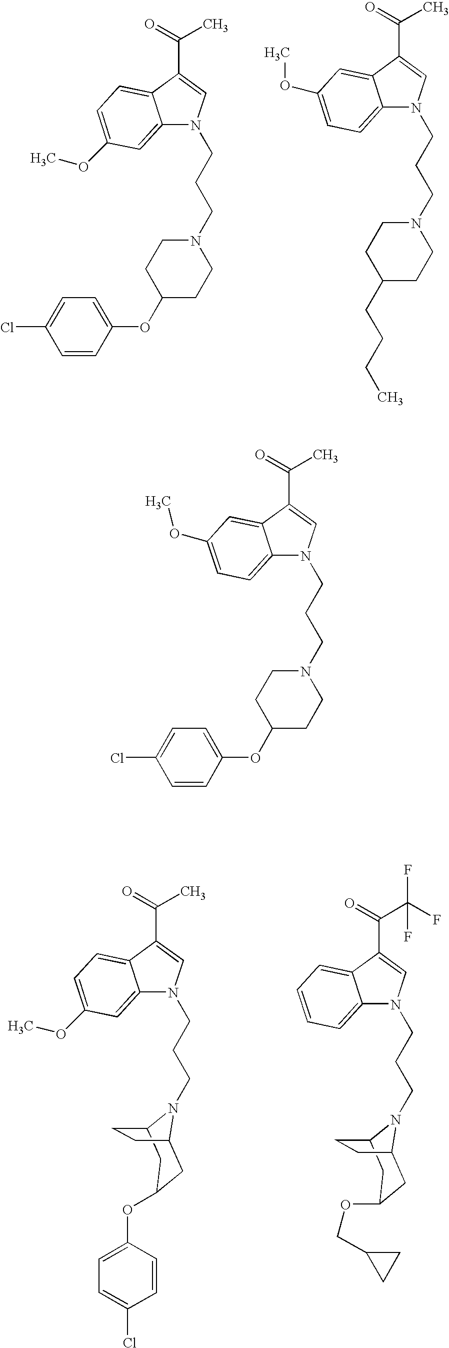

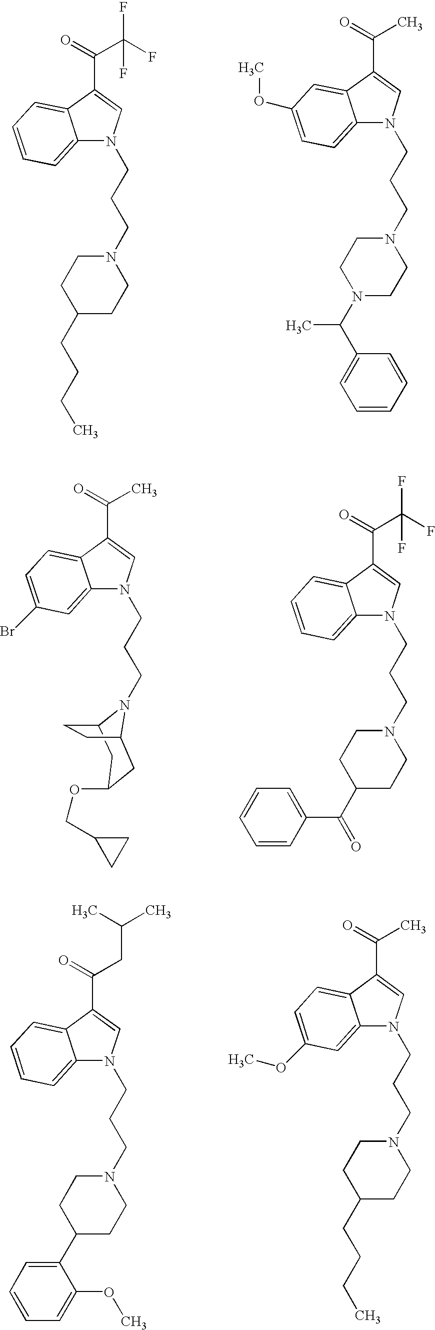

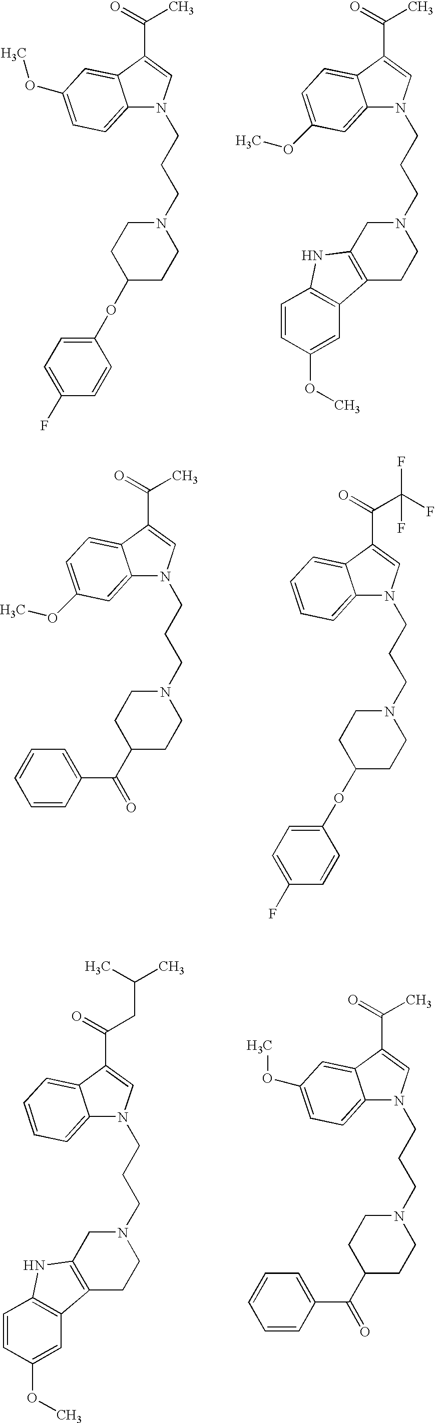

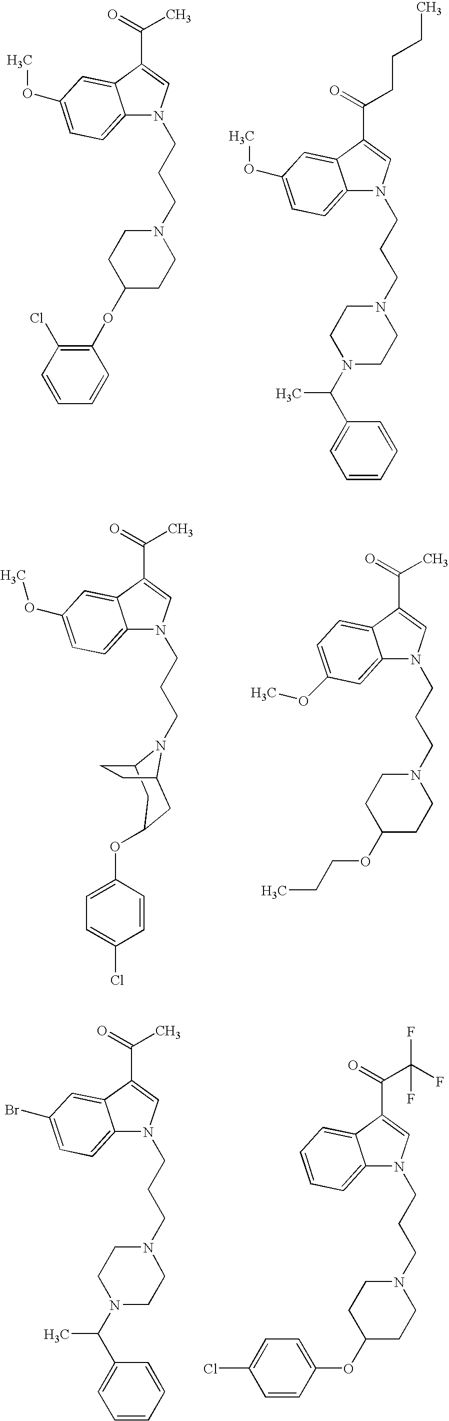

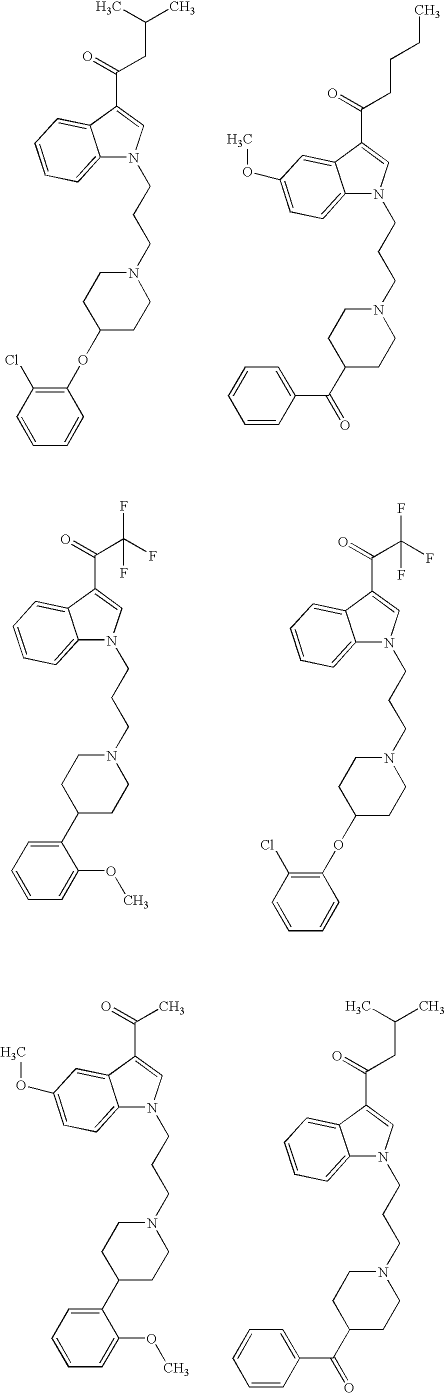

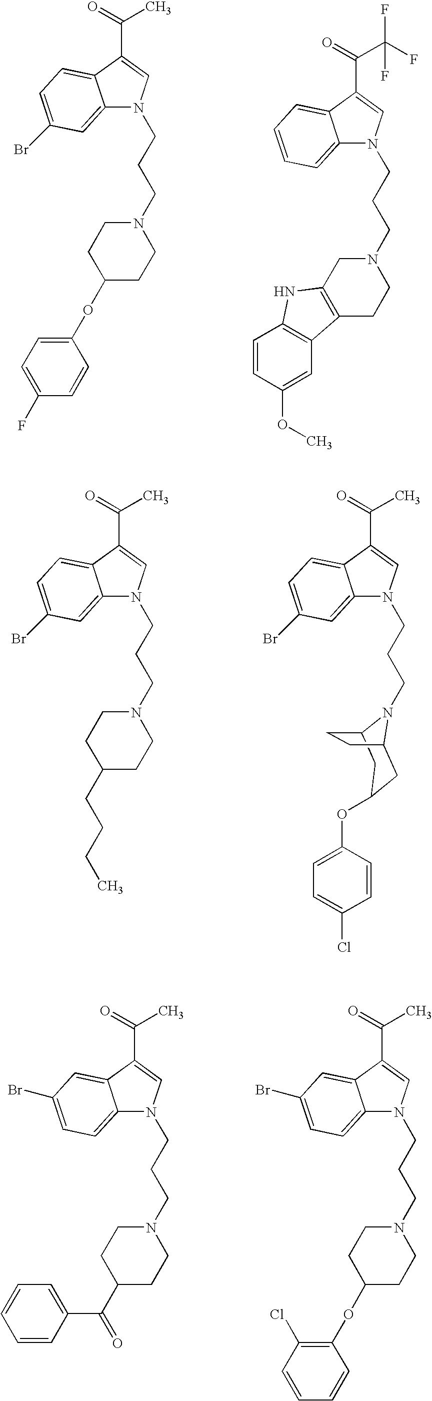

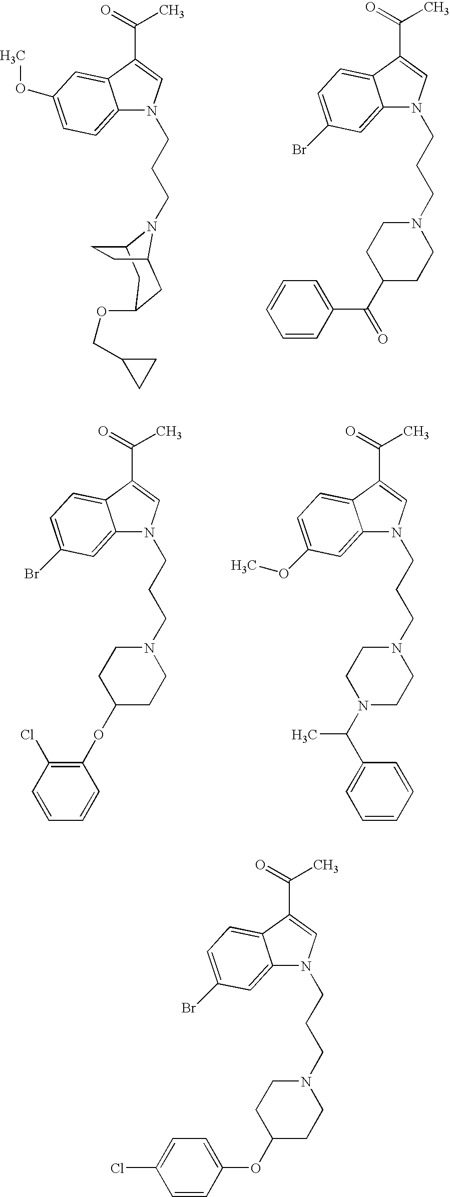

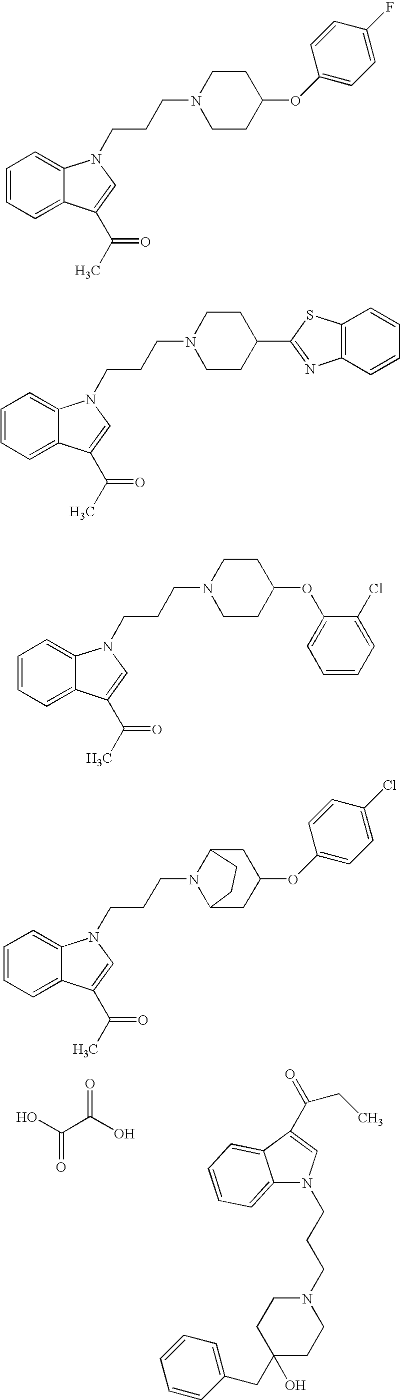

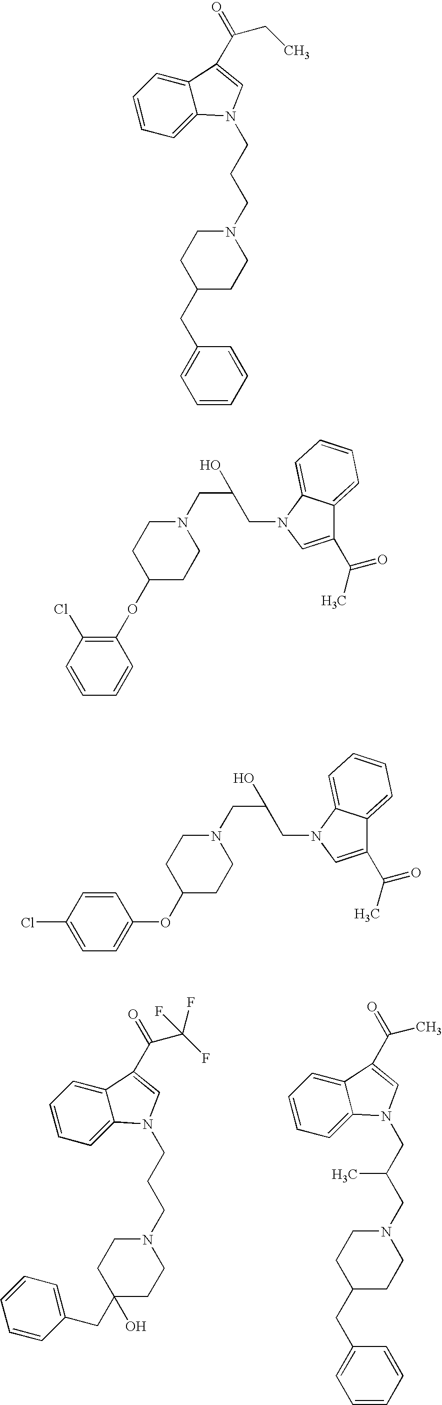

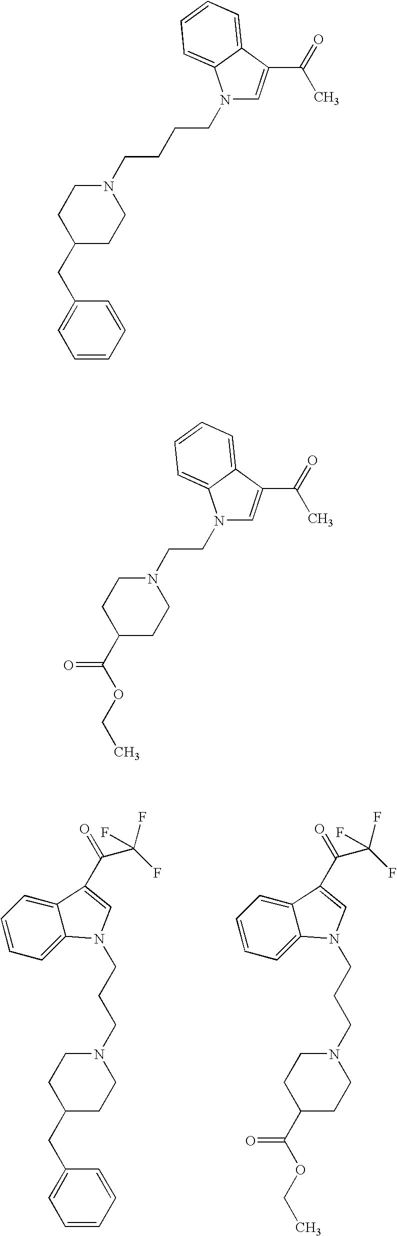

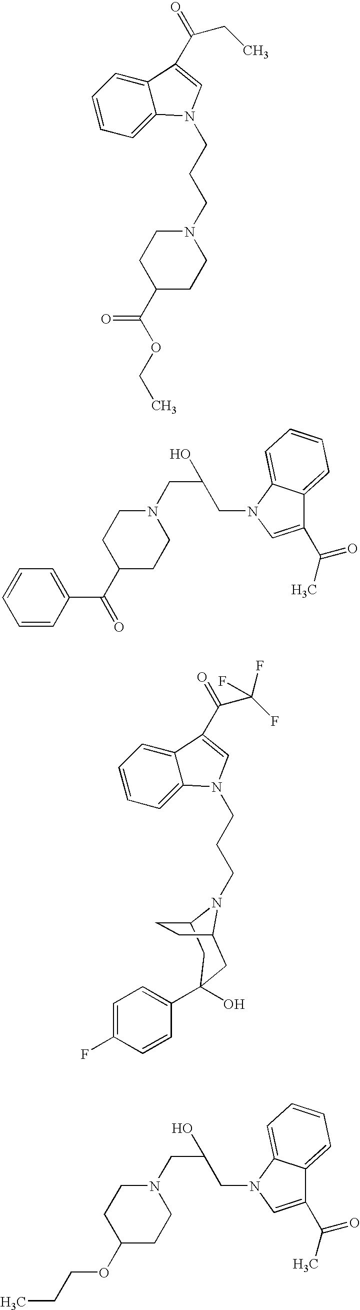

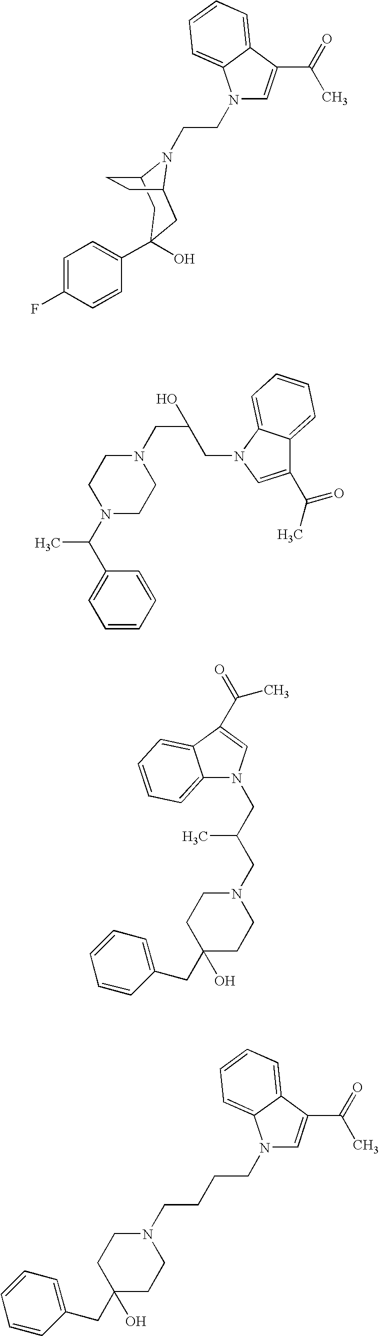

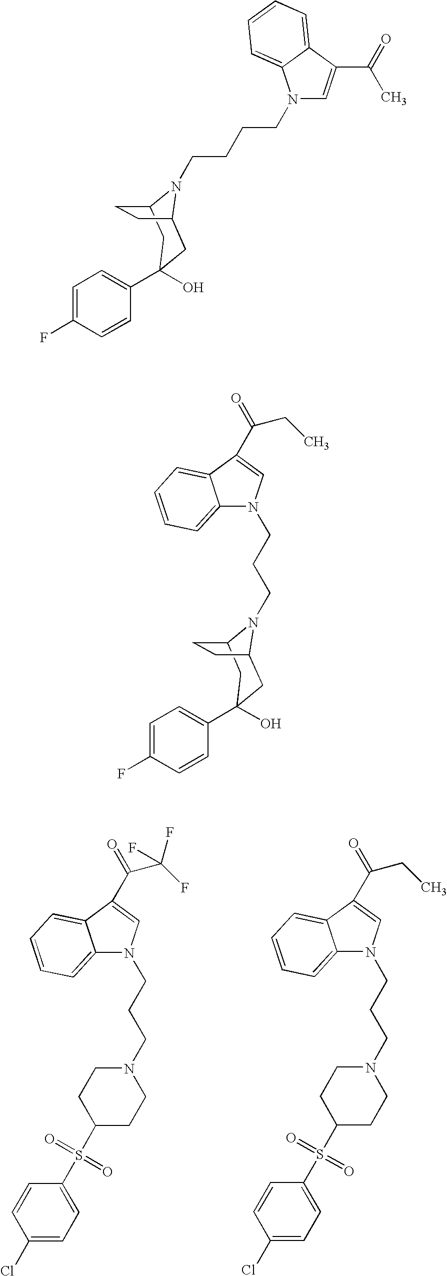

62. The compound of claim 1, wherein the compound is selected from the group consisting of:

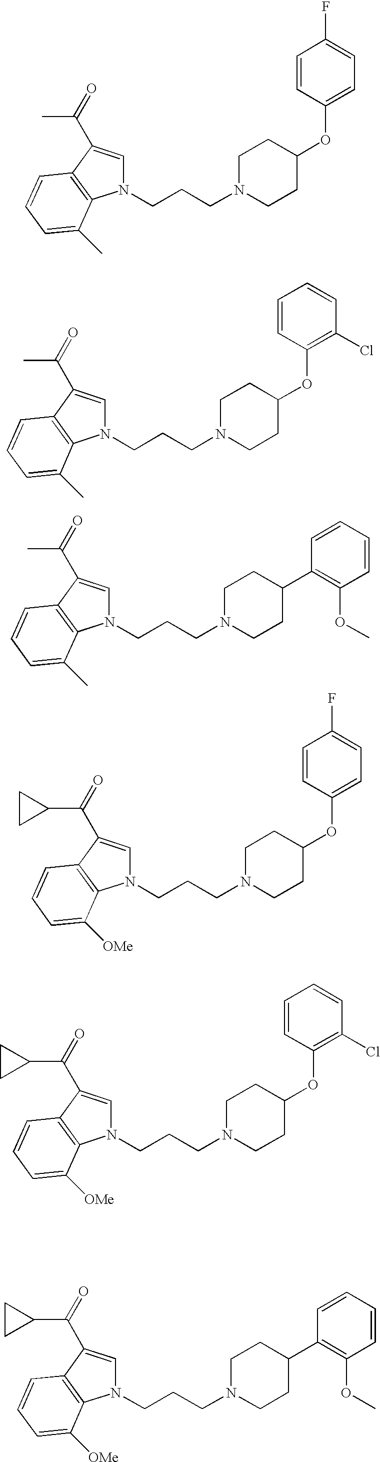

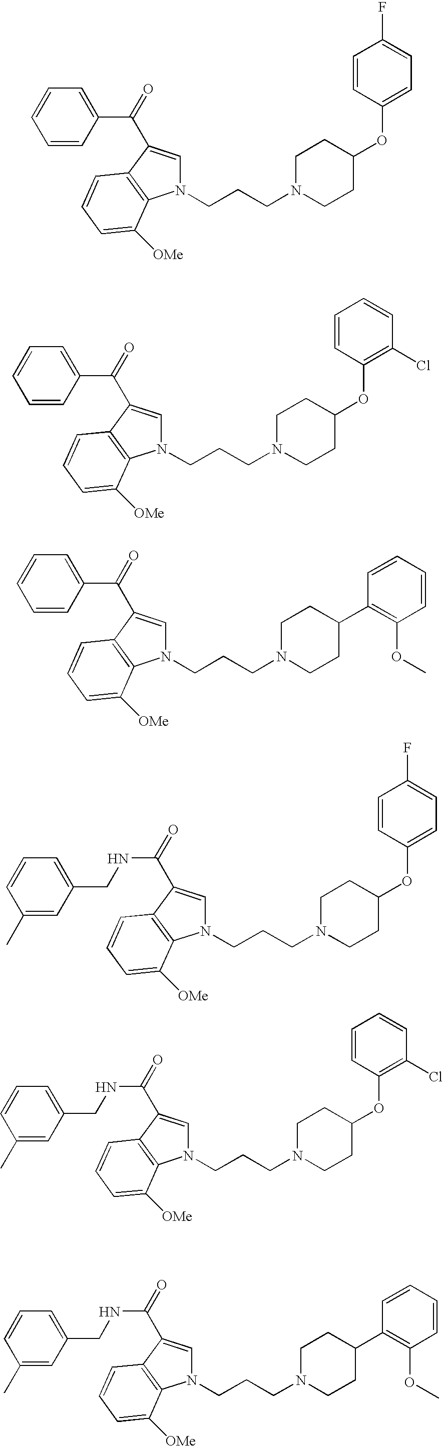

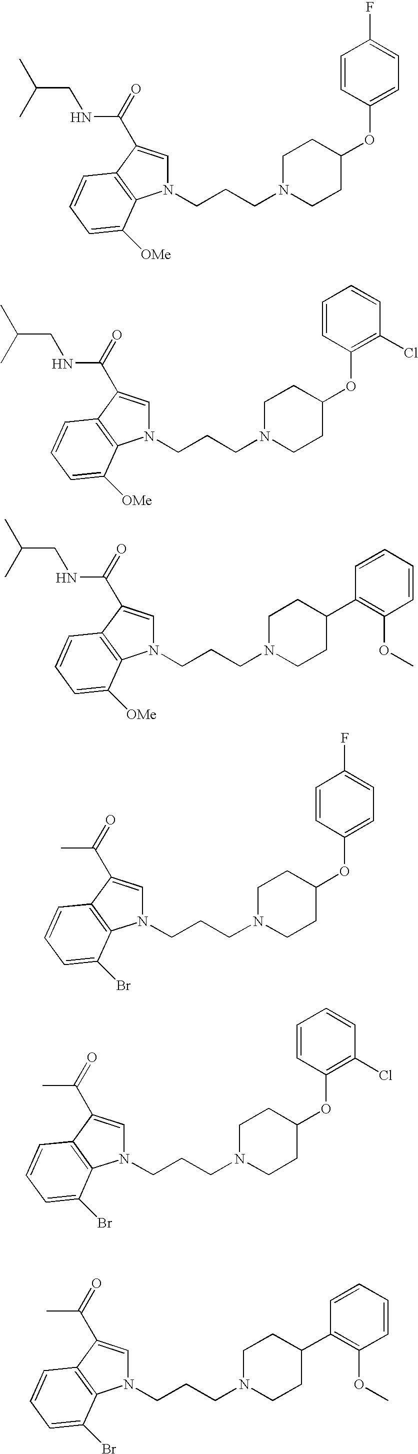

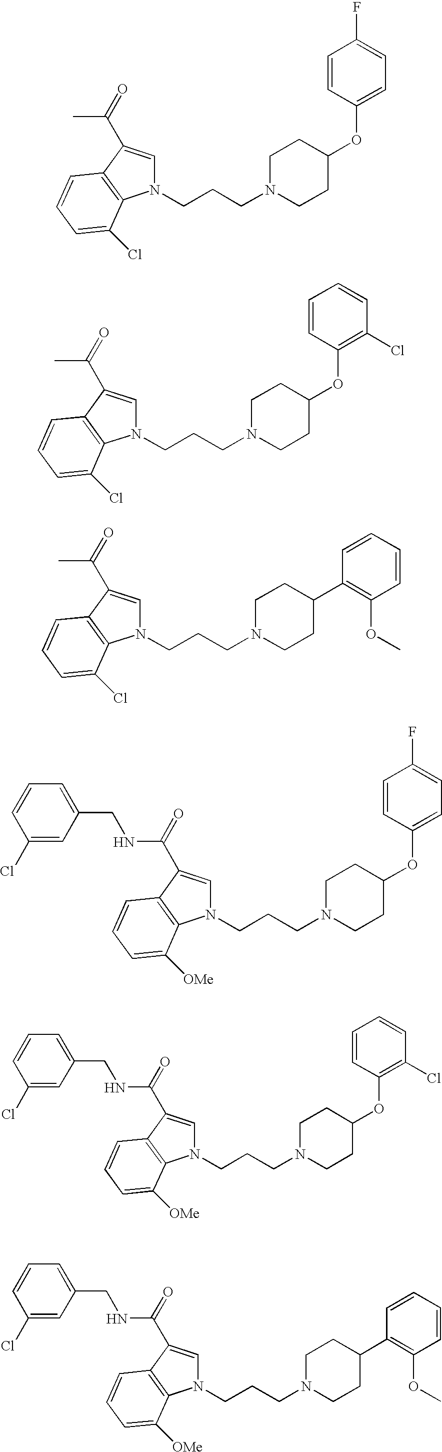

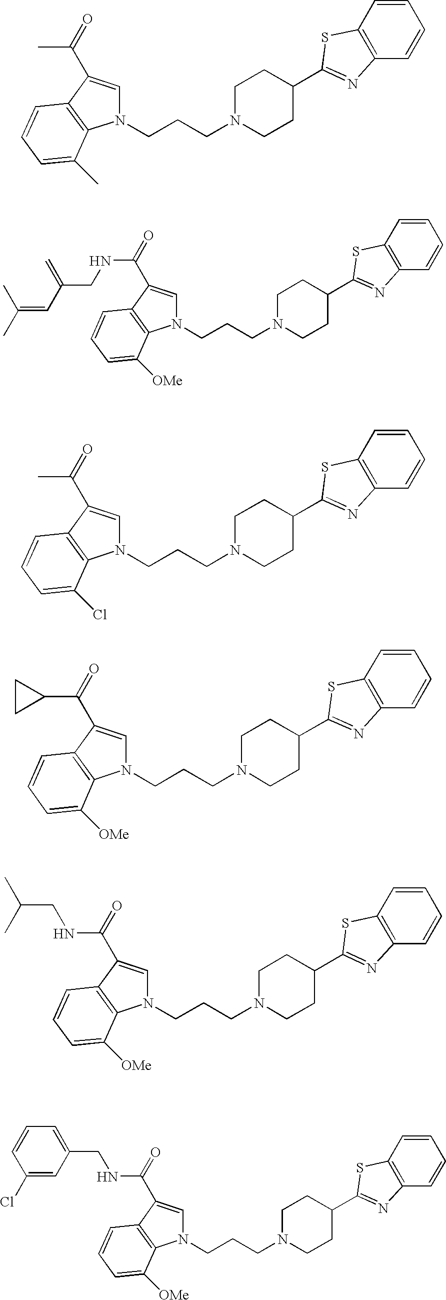

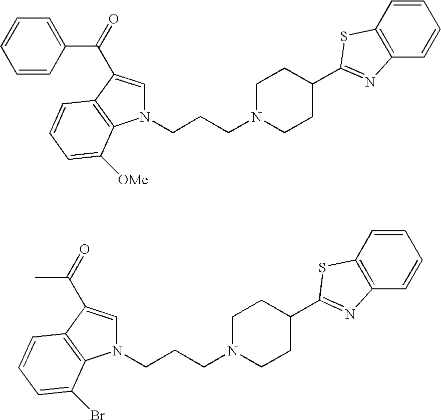

63. The compound of claim 1, wherein the compound is selected from the group consisting of:

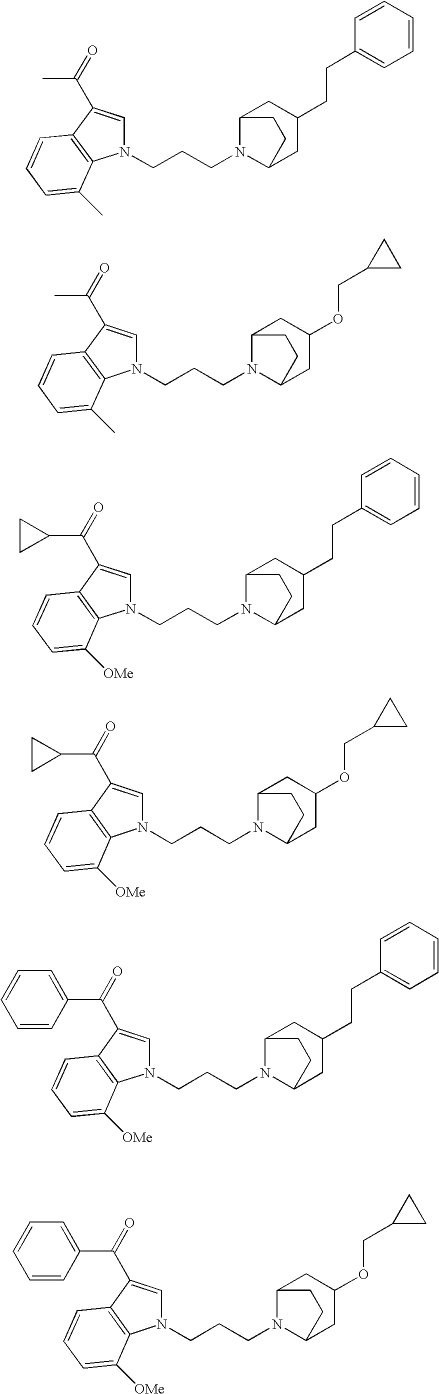

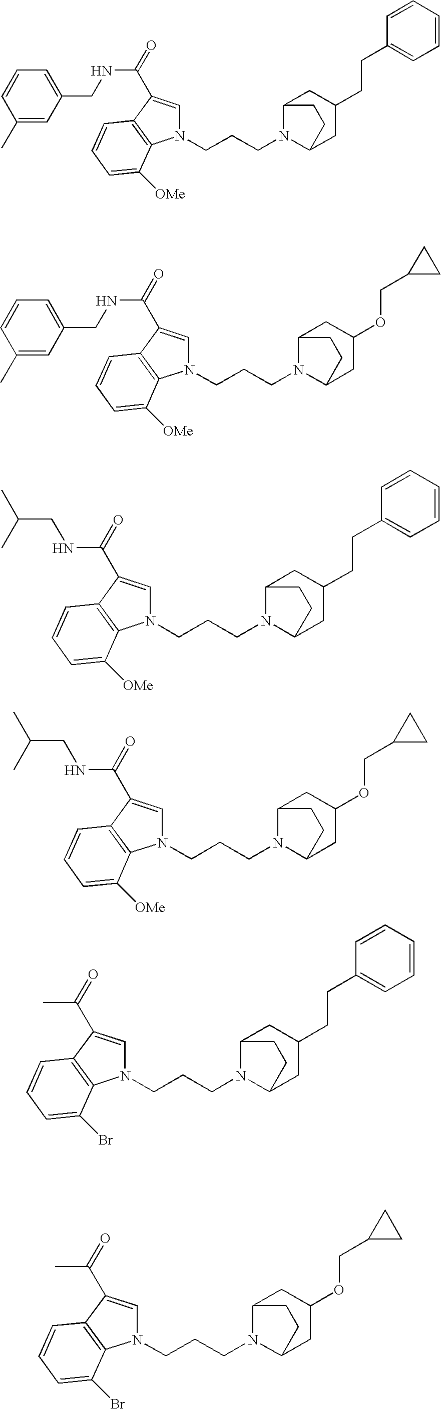

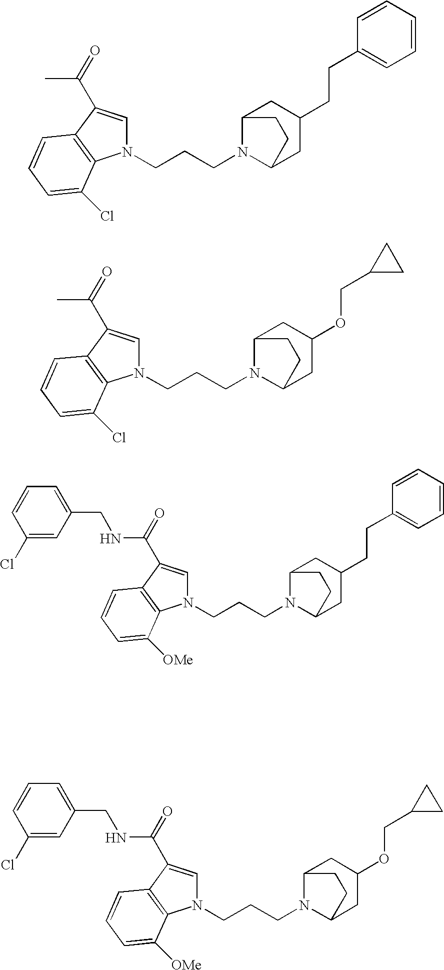

64. The compounds of claim 63, wherein the compounds have the following stereochemistry:

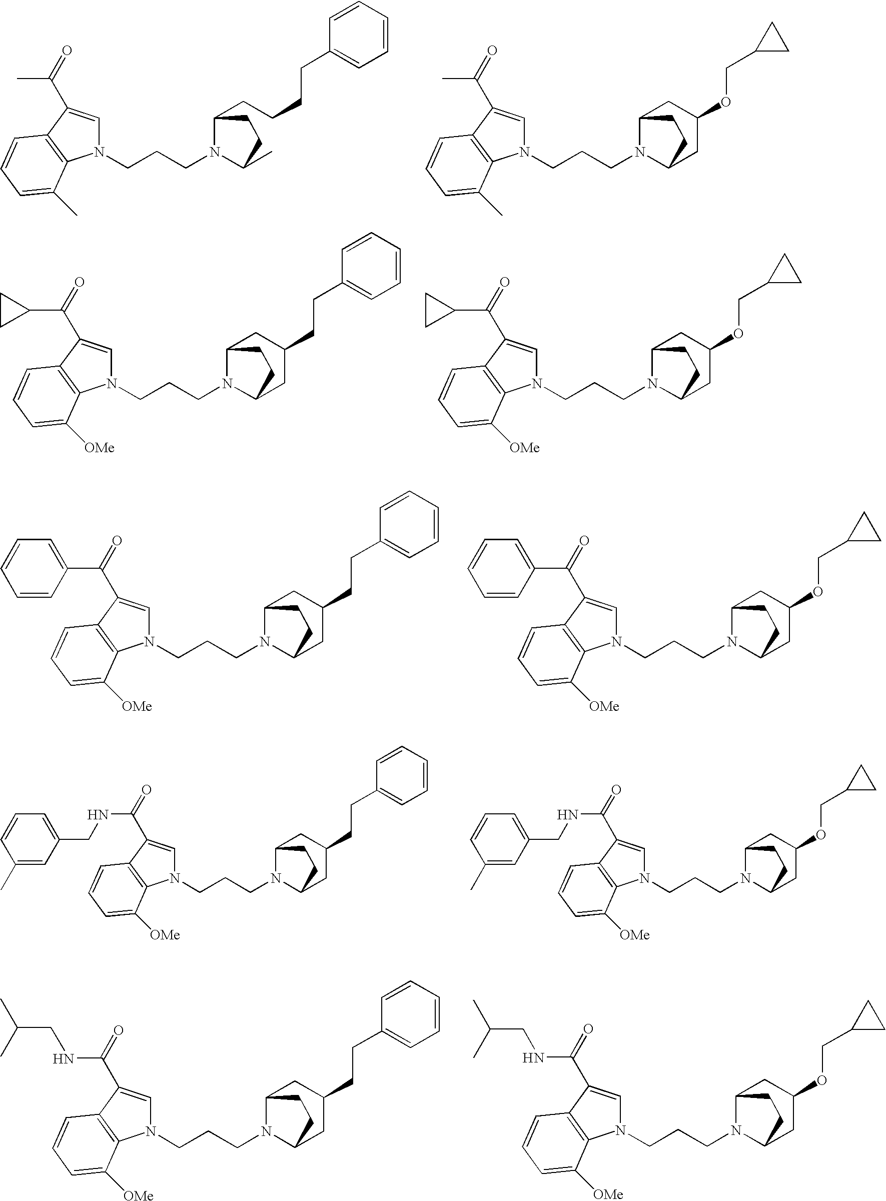

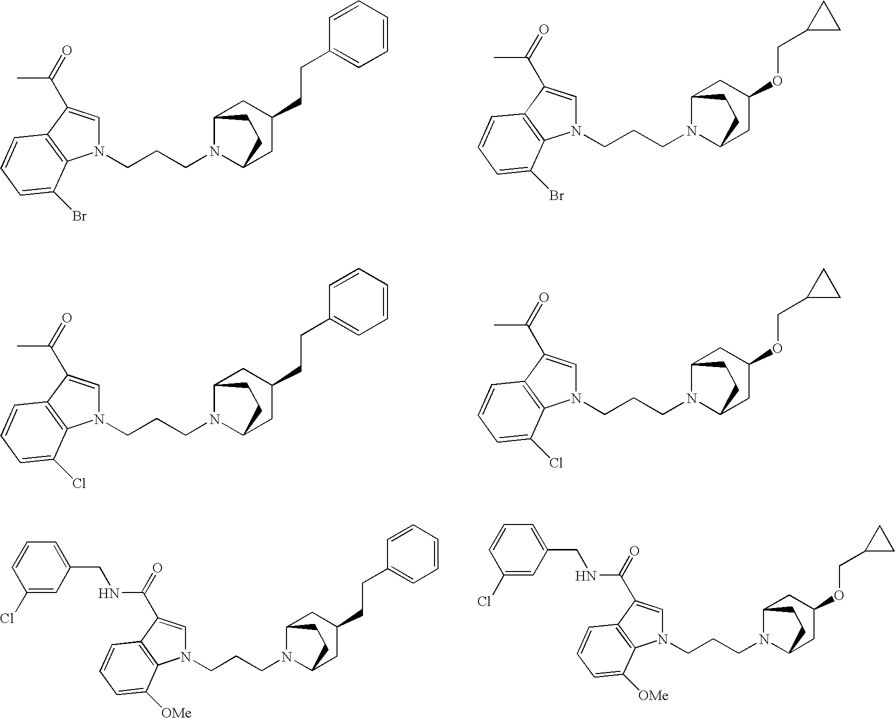

65. The compounds of claim 63, wherein the compound is selected from the group consisting of:

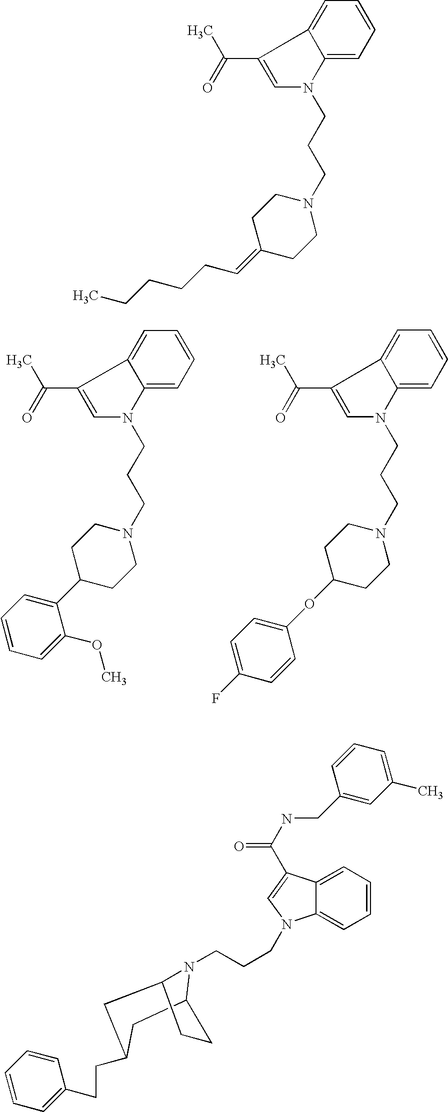

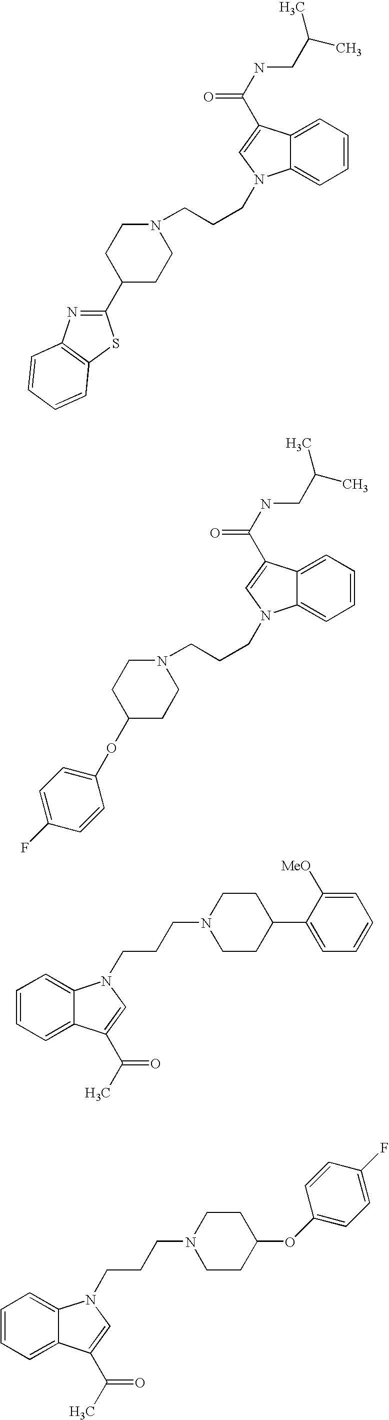

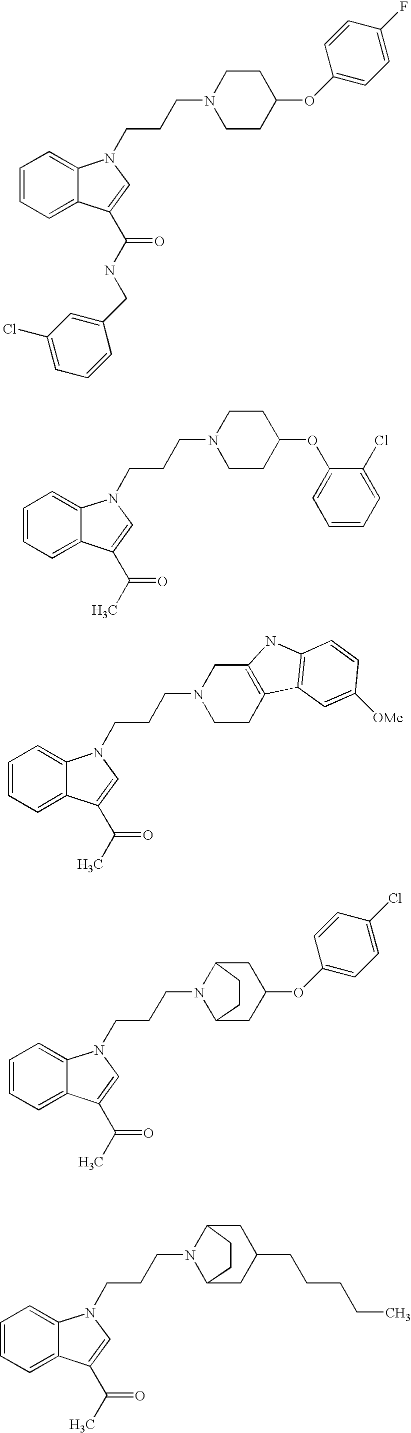

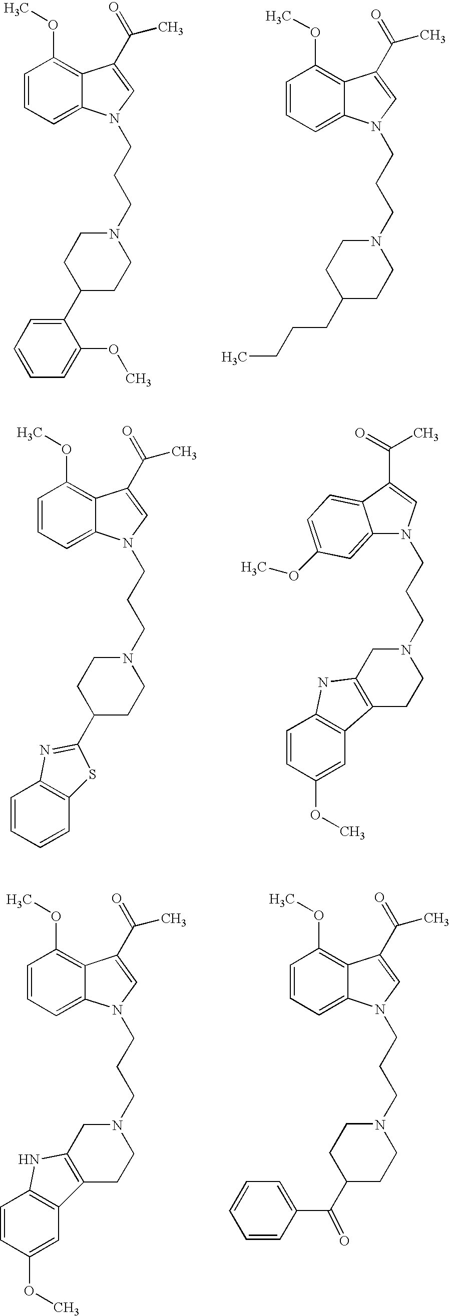

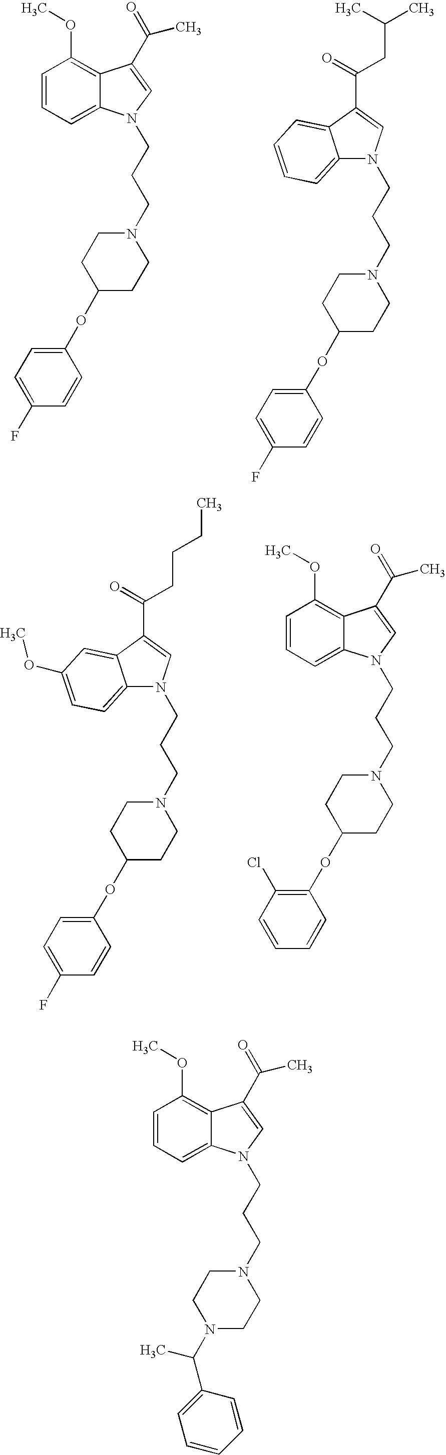

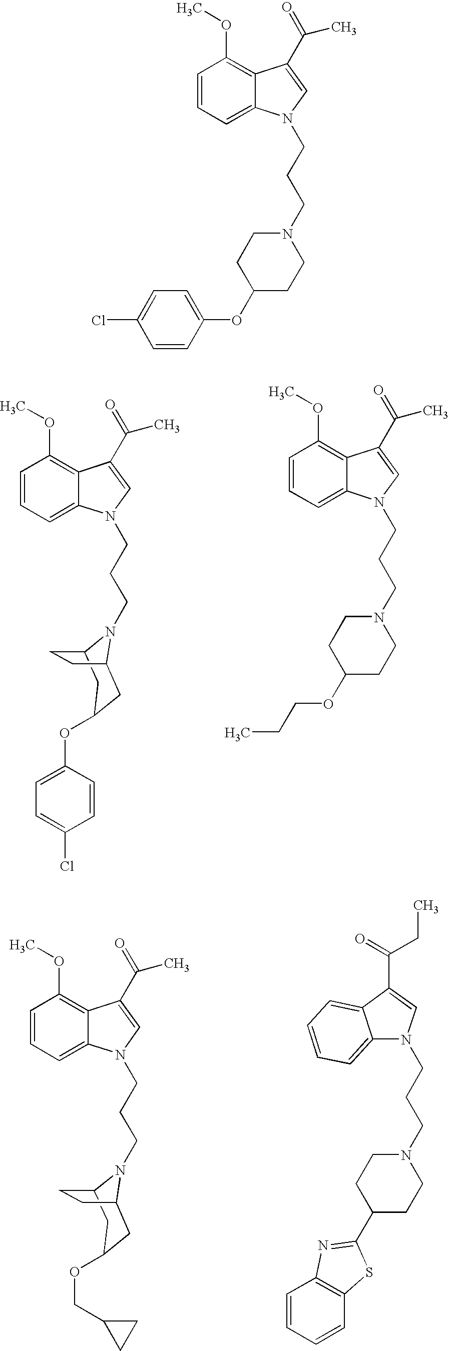

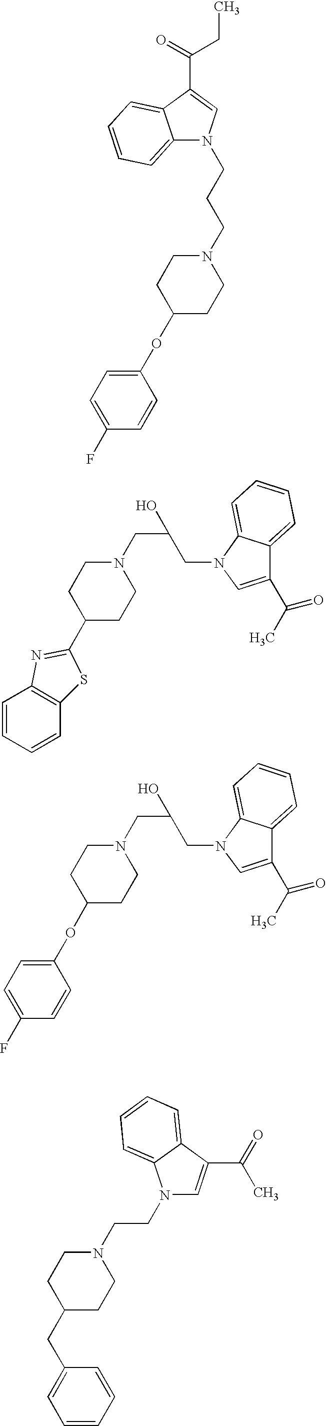

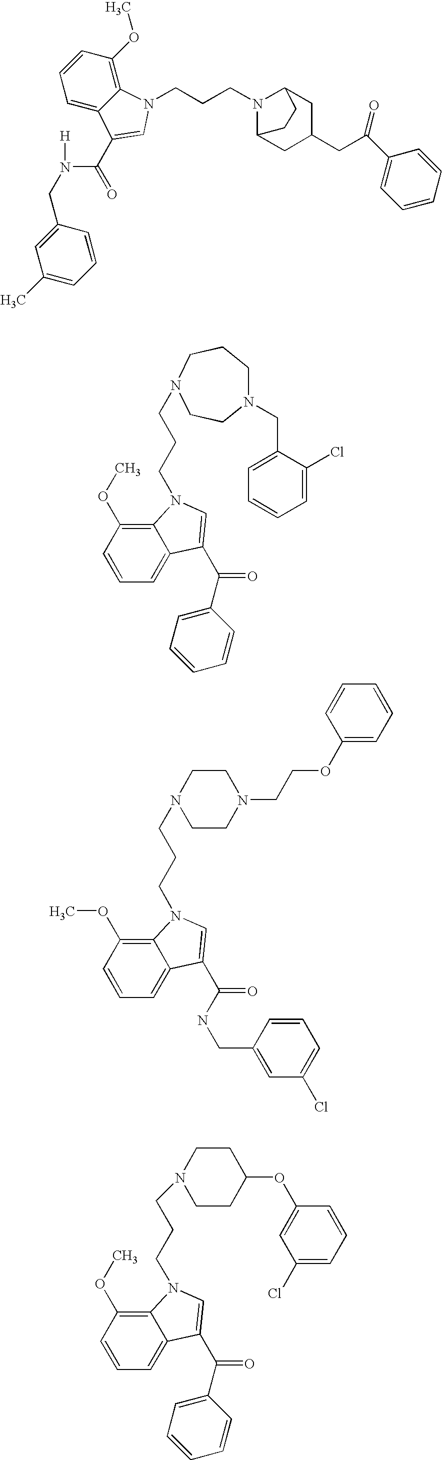

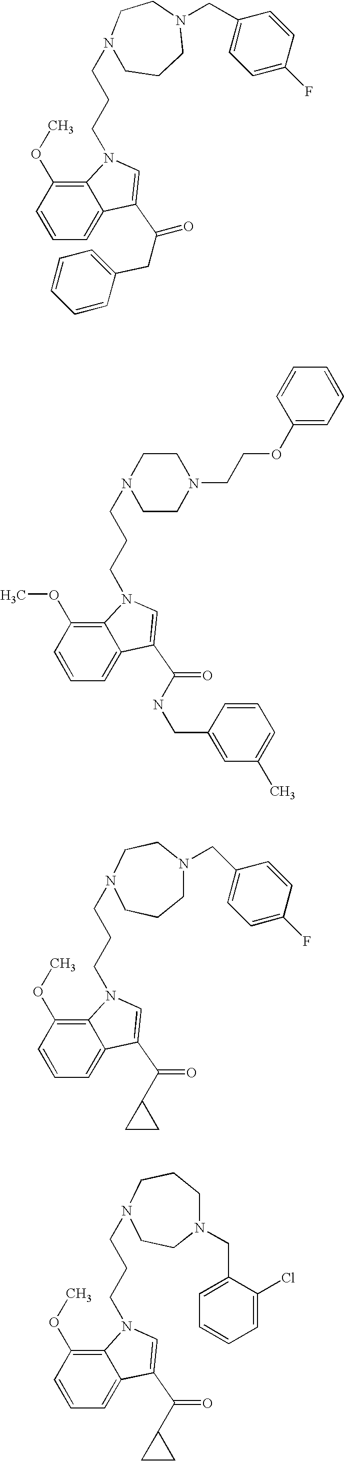

66. The compound of claim 1, wherein the compound is selected from the group consisting of:

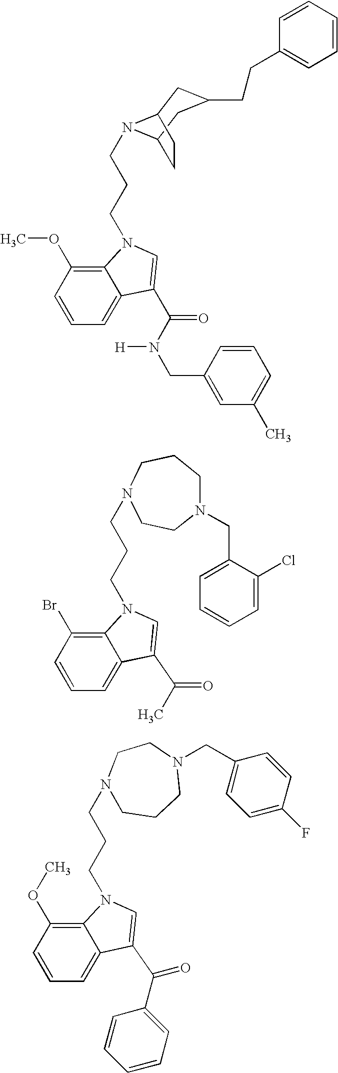

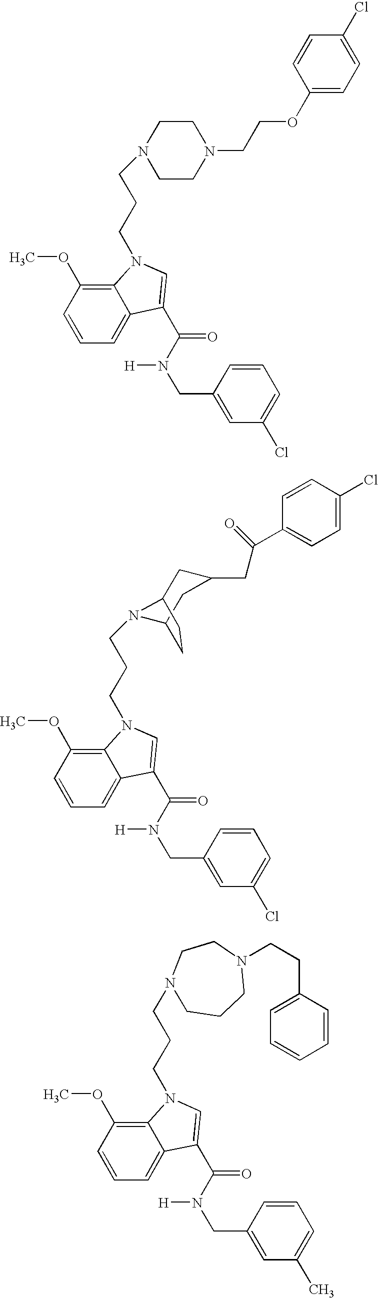

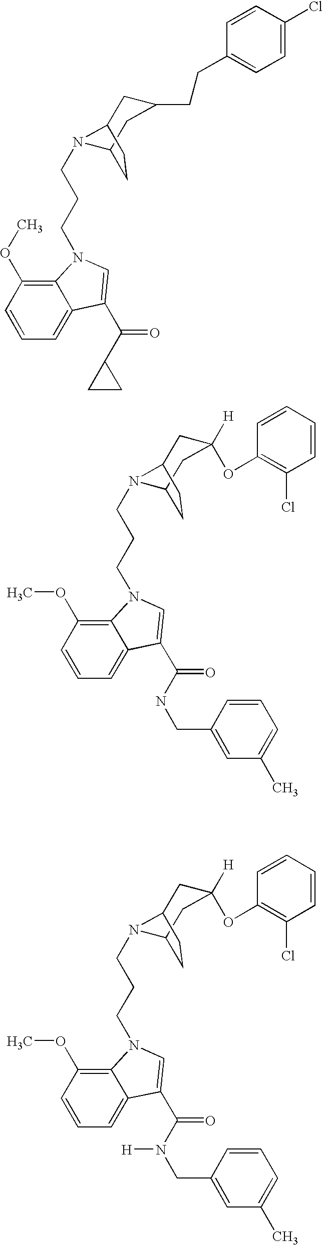

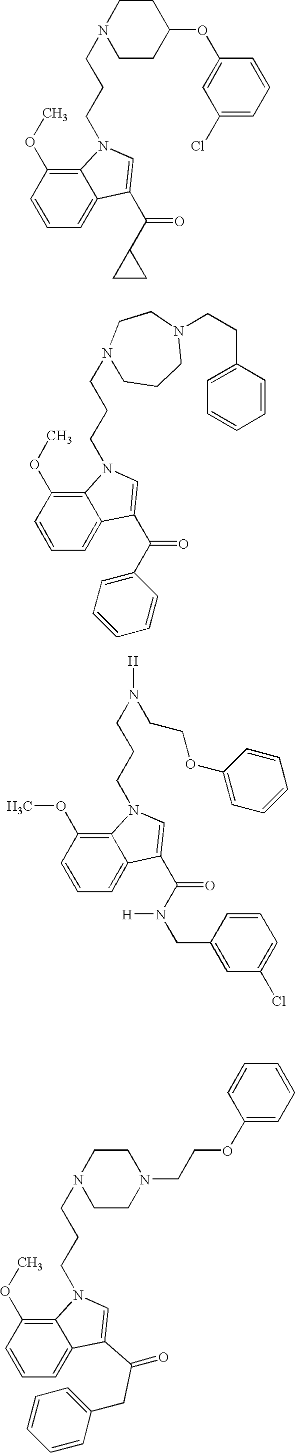

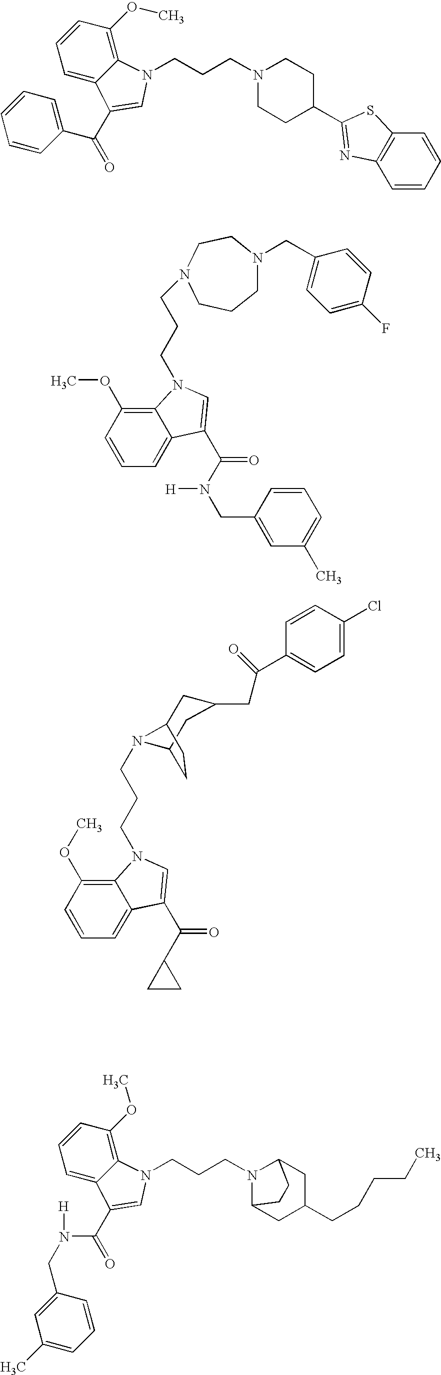

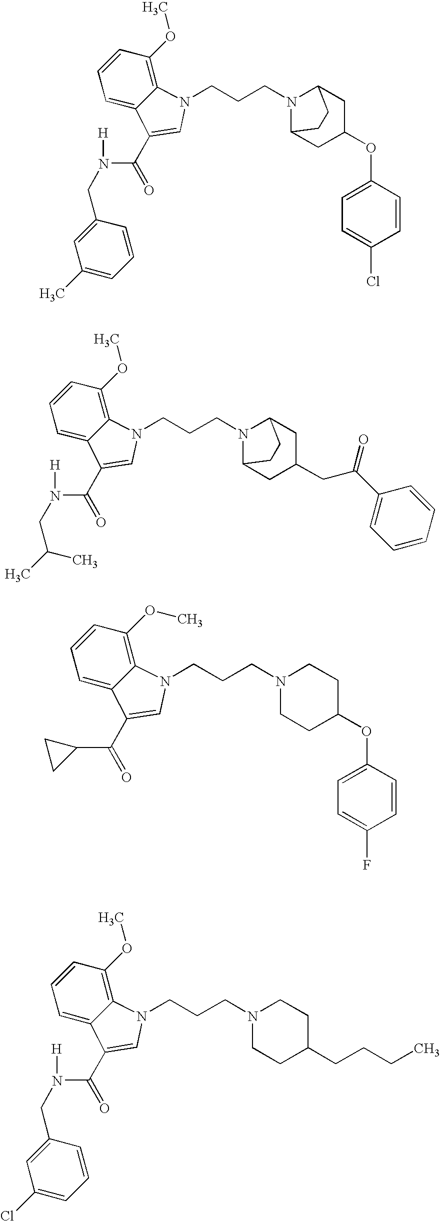

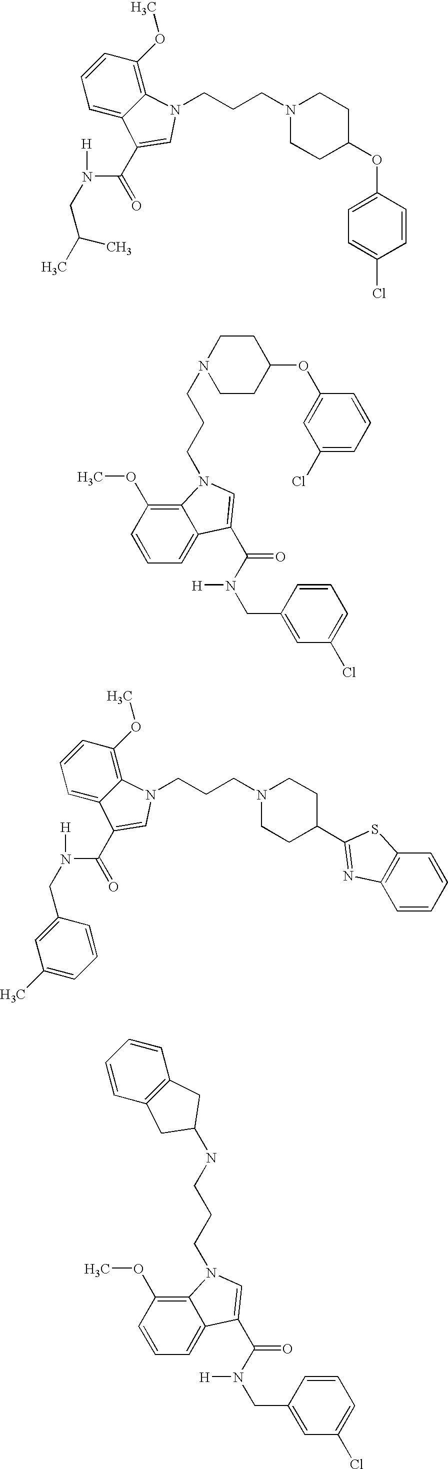

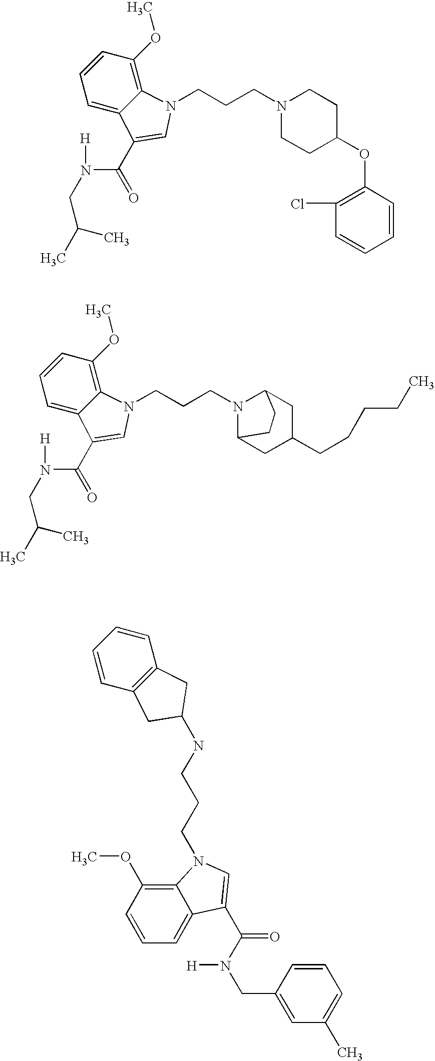

67. The compound of claim 1, wherein the compound is selected from the group consisting of:

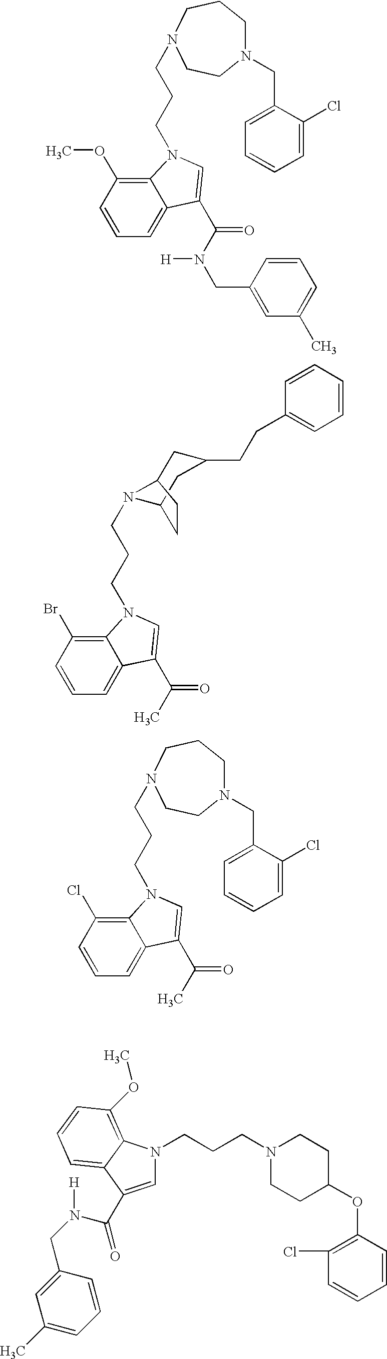

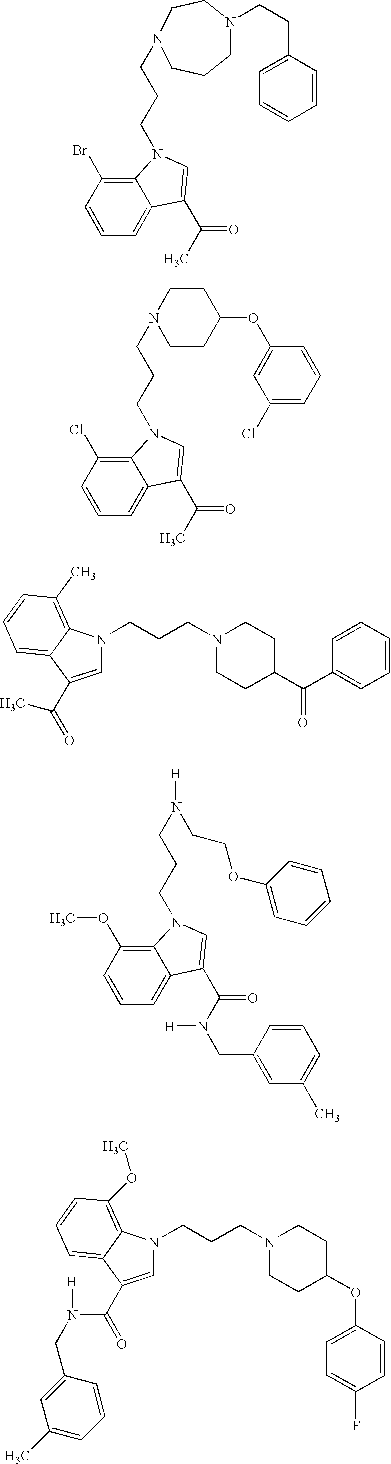

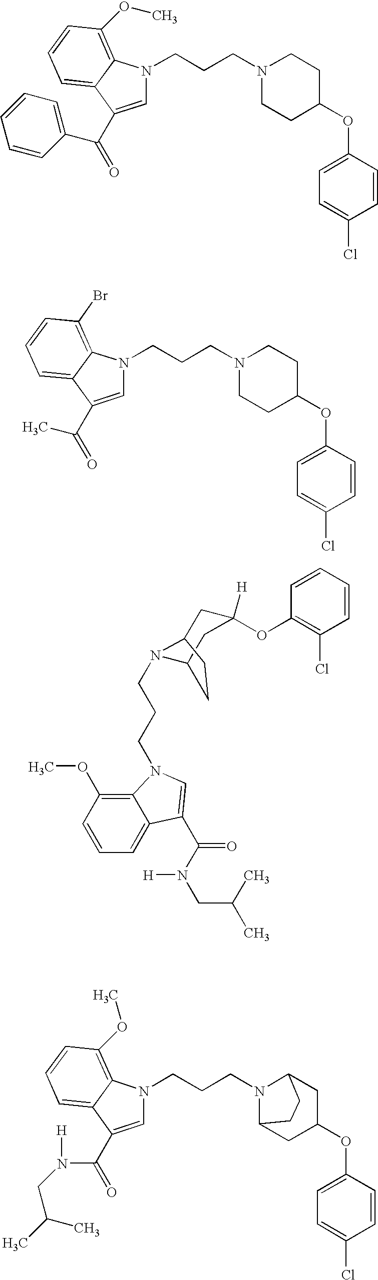

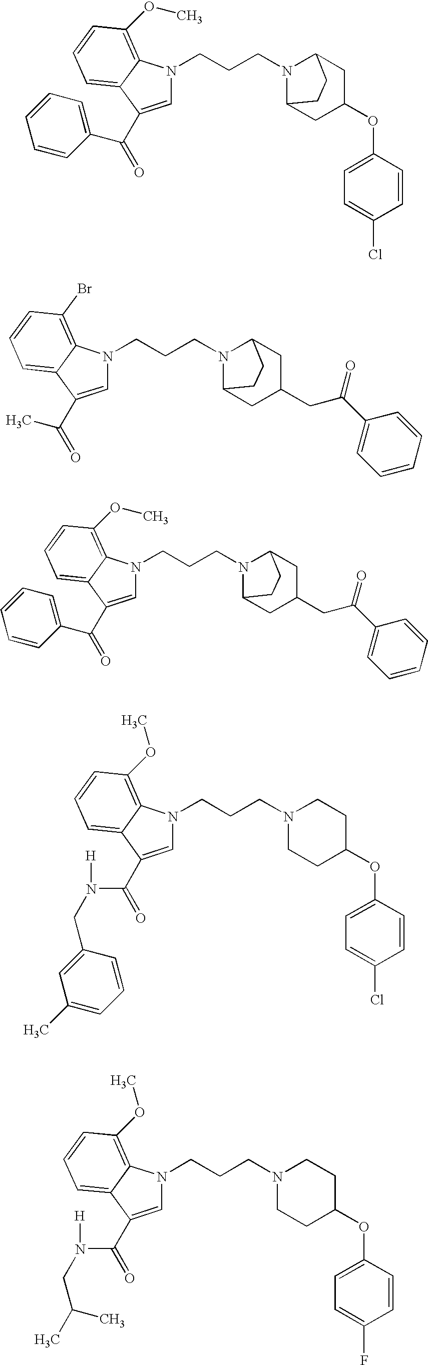

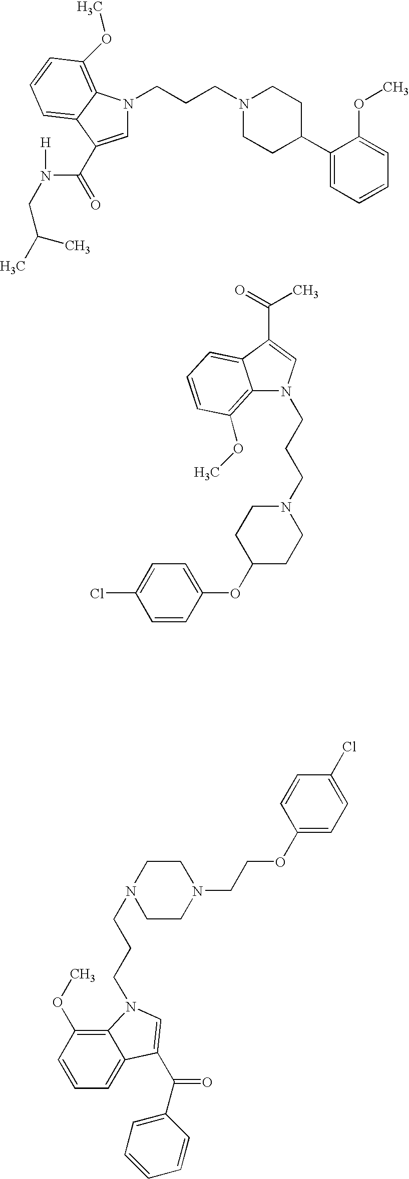

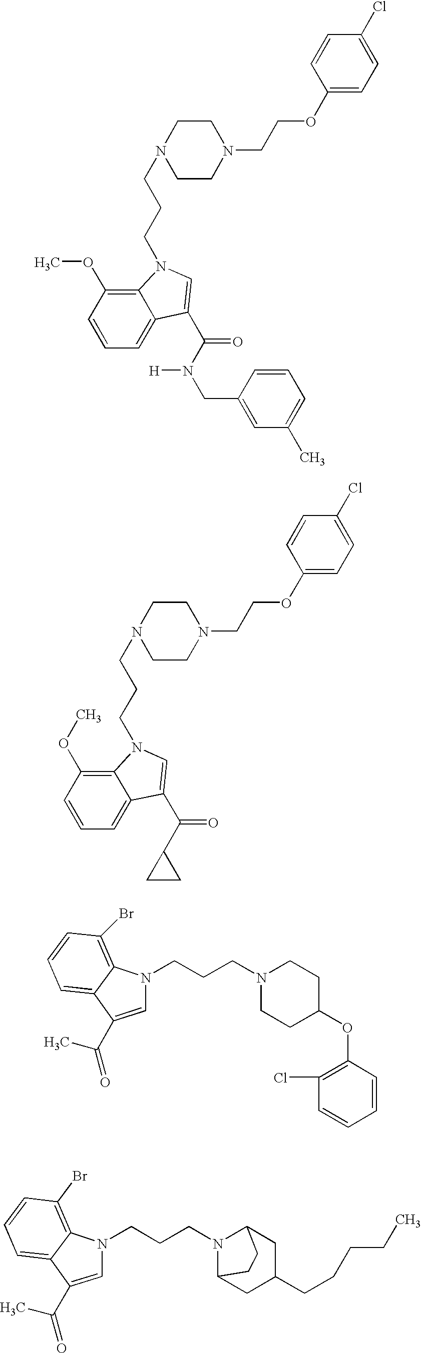

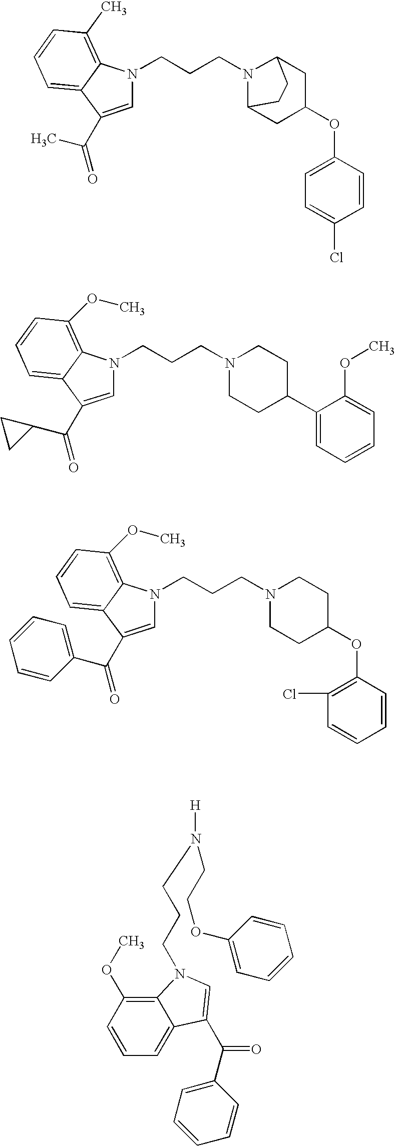

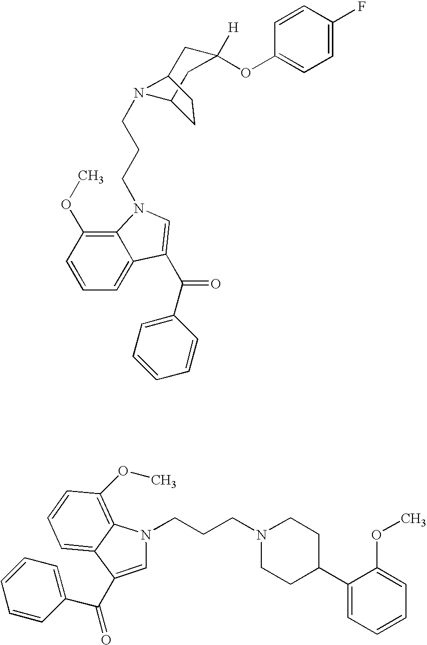

68. The compound of claim 1, wherein the compound is selected from the group consisting of:

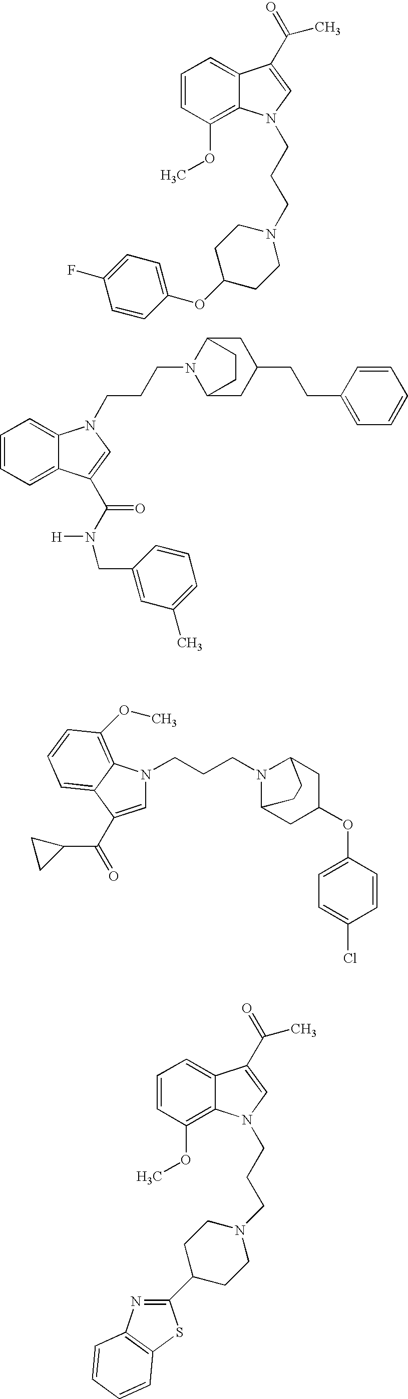

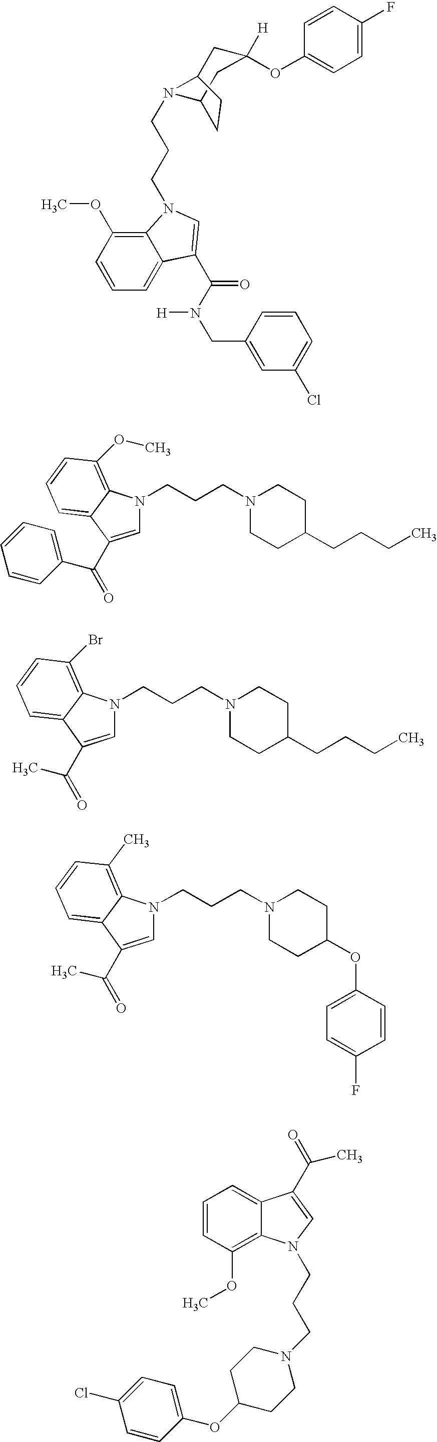

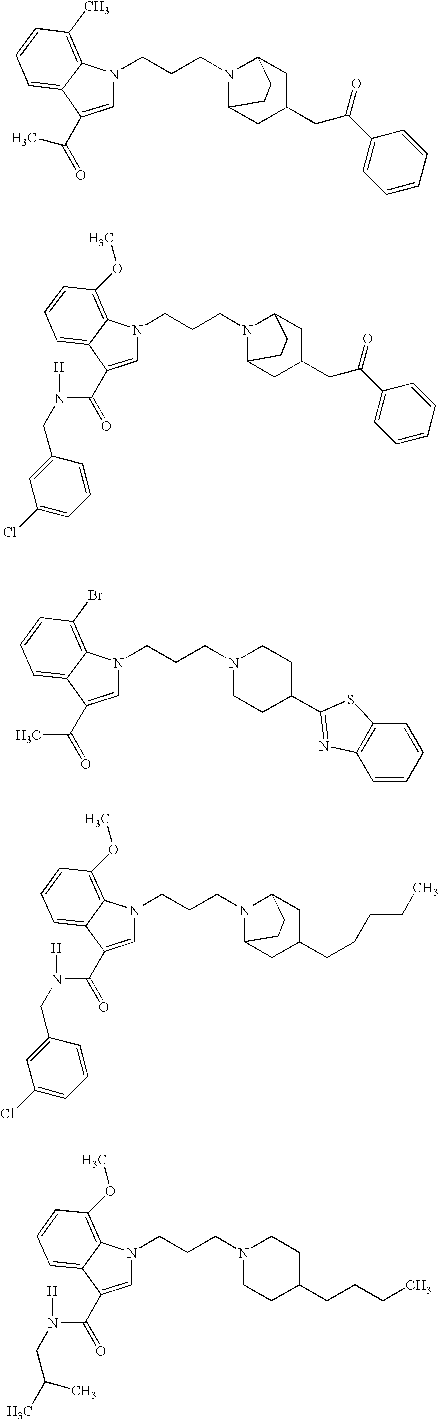

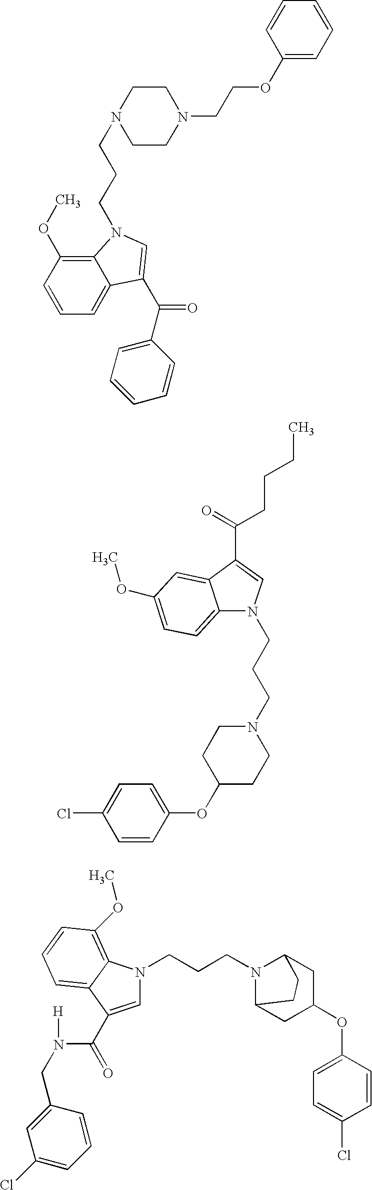

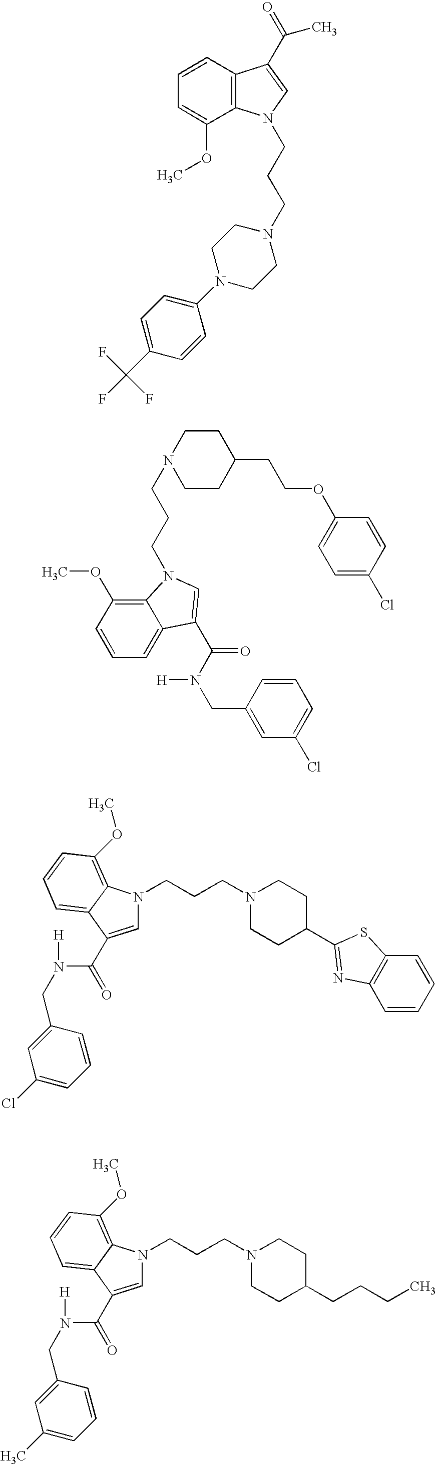

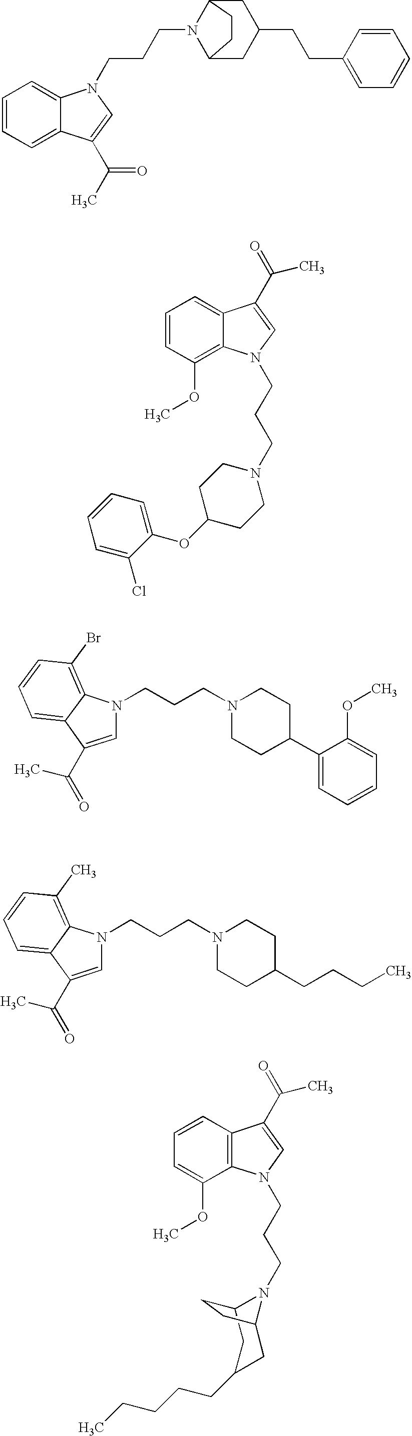

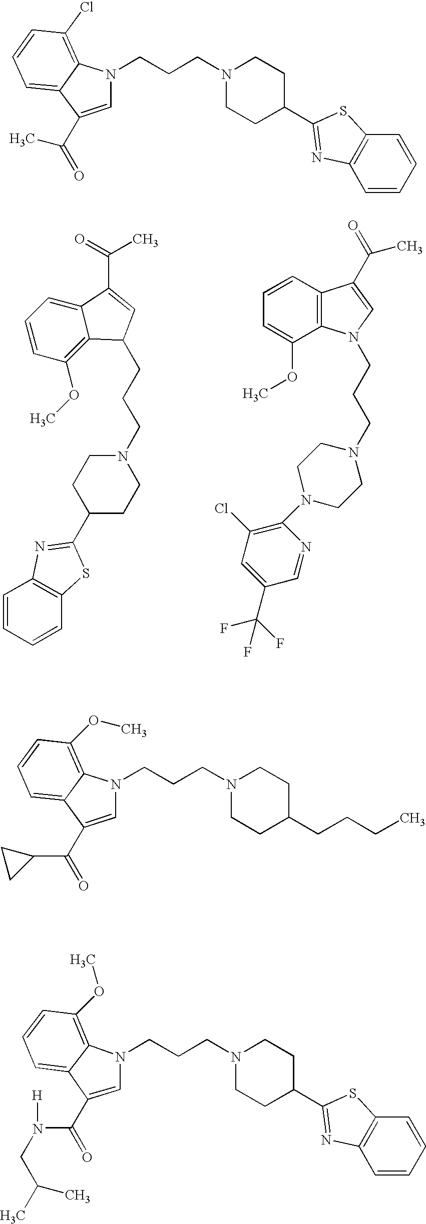

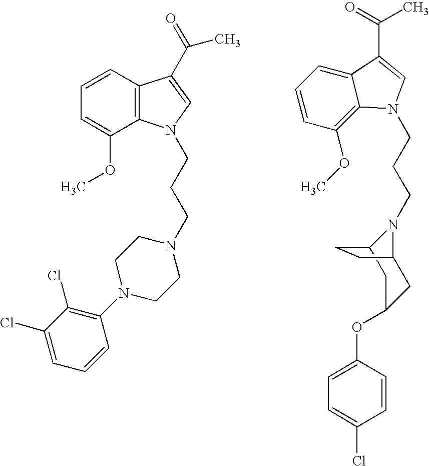

69. The compound of claim 1, wherein the compound is selected from the group consisting of:

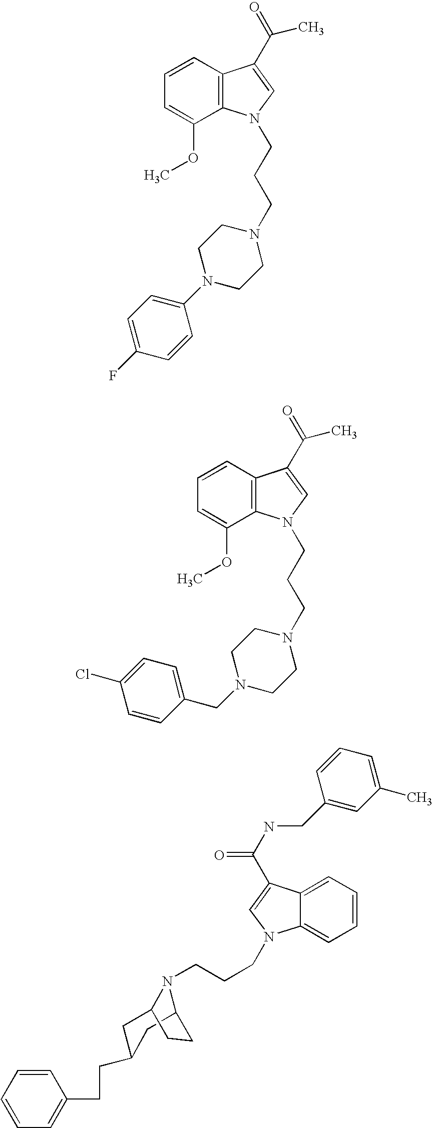

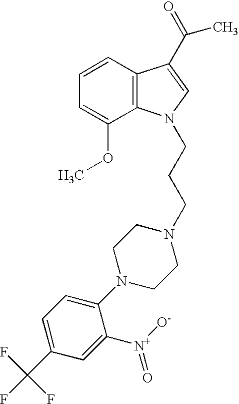

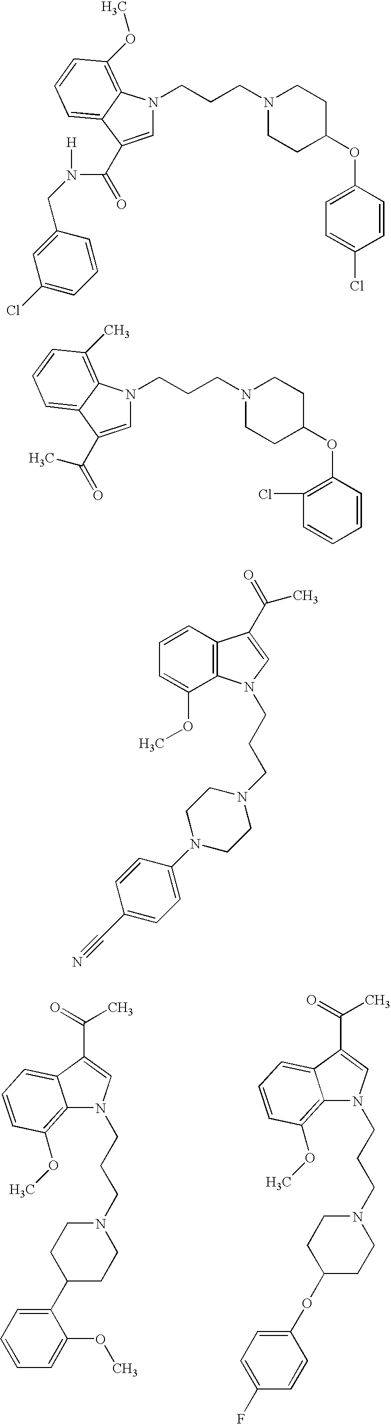

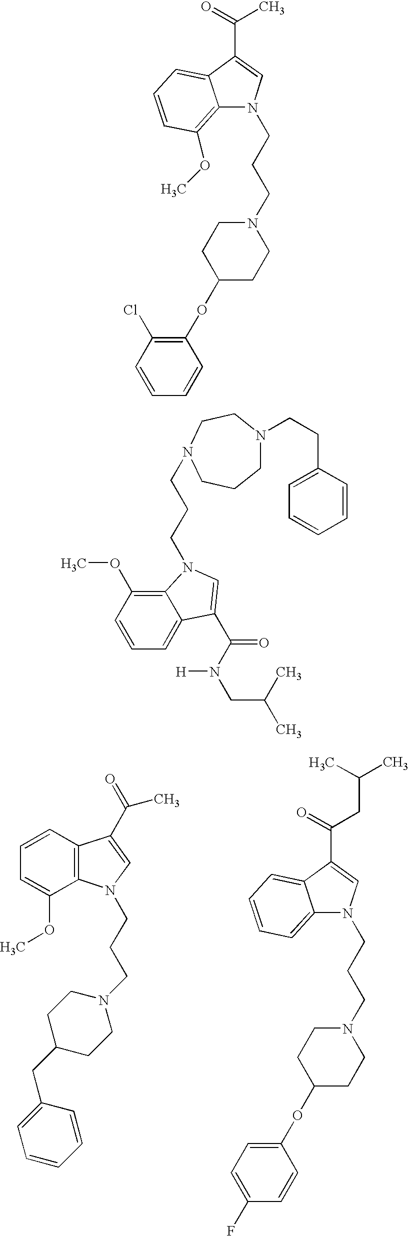

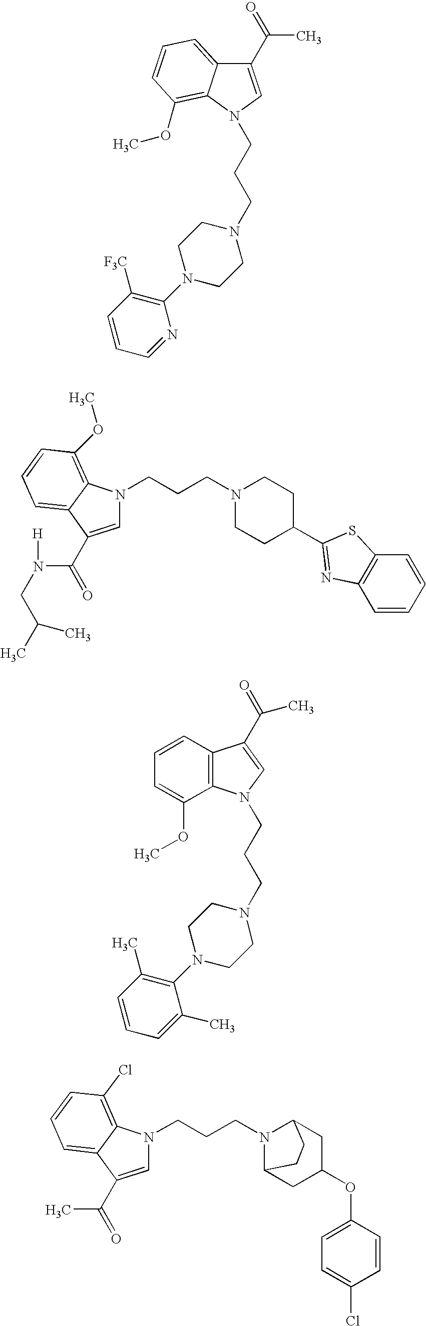

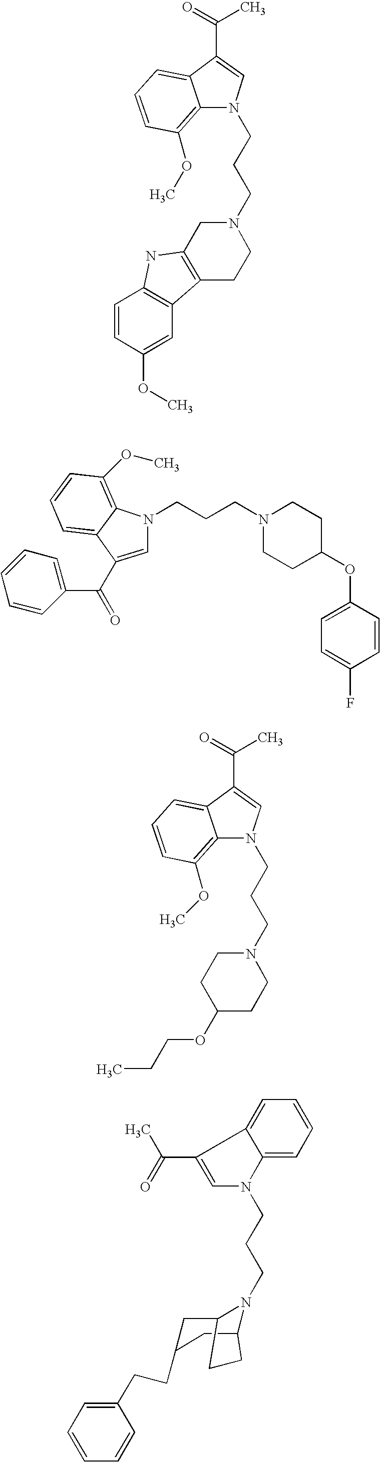

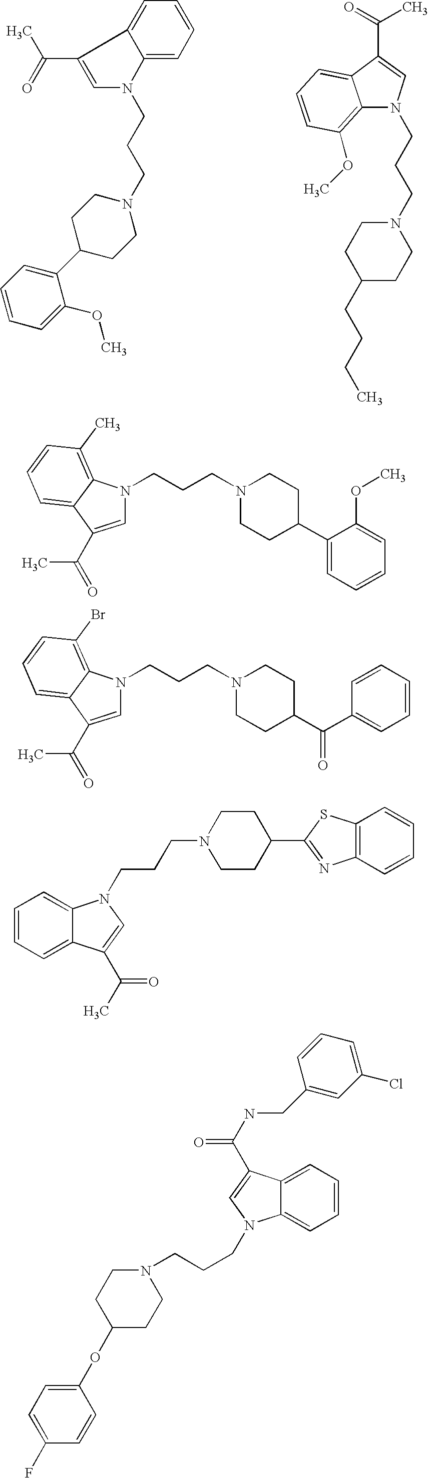

70. The compound of claim 1, wherein the compound is selected from the group consisting of:

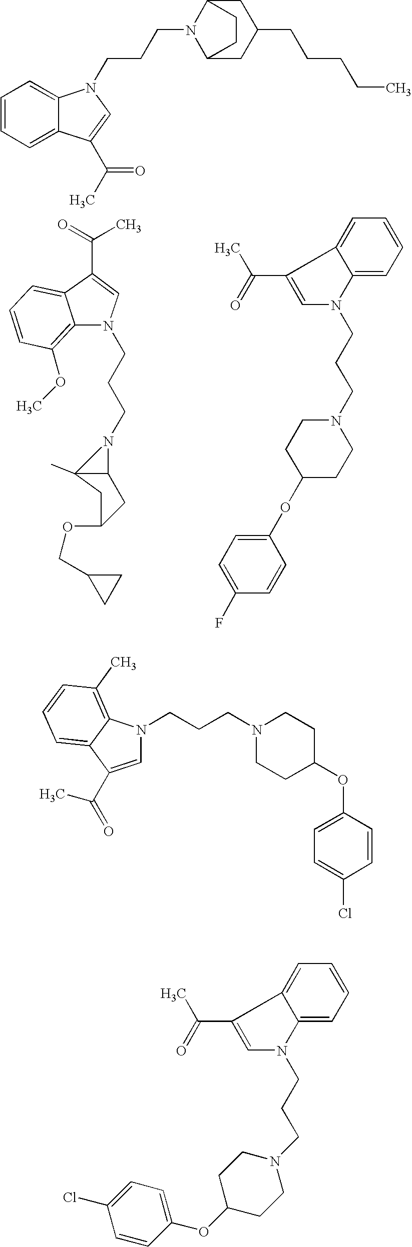

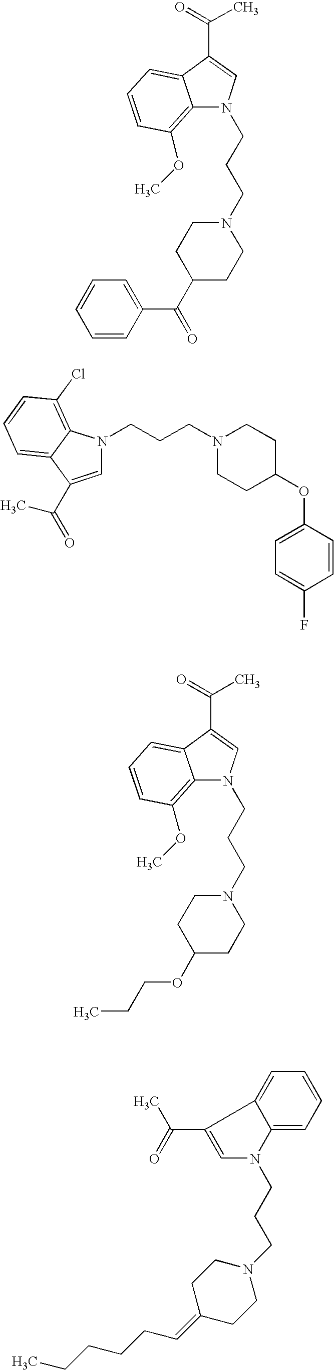

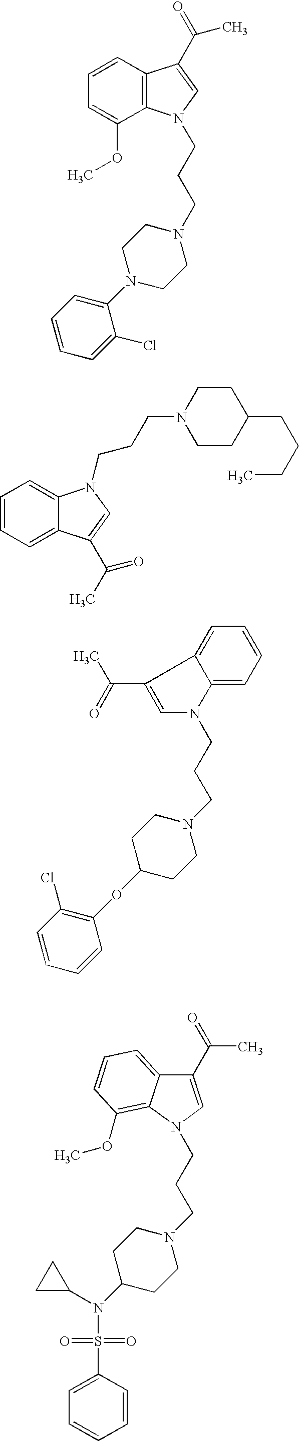

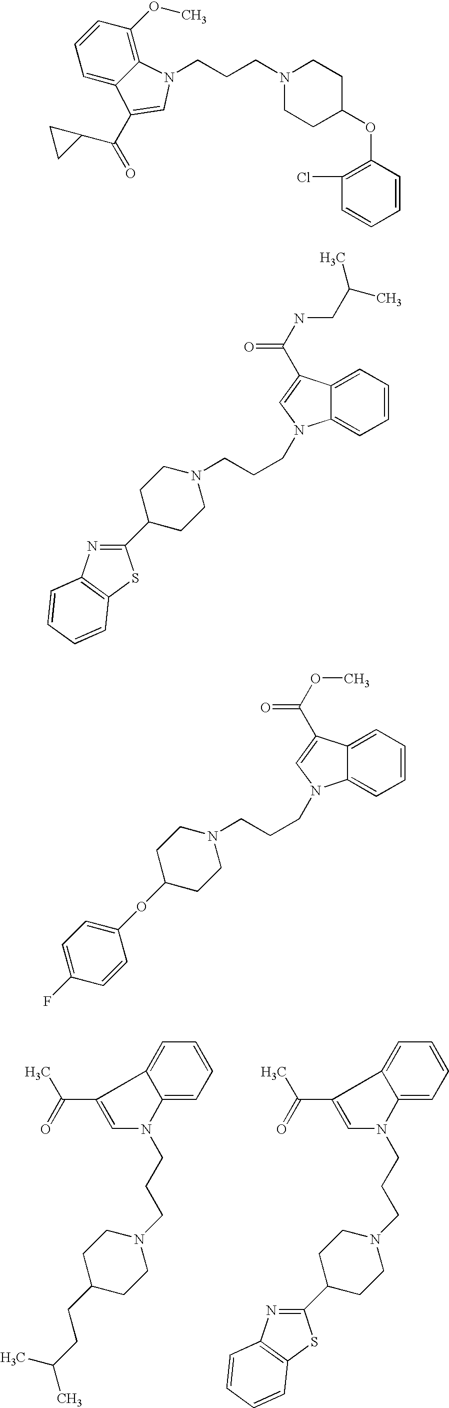

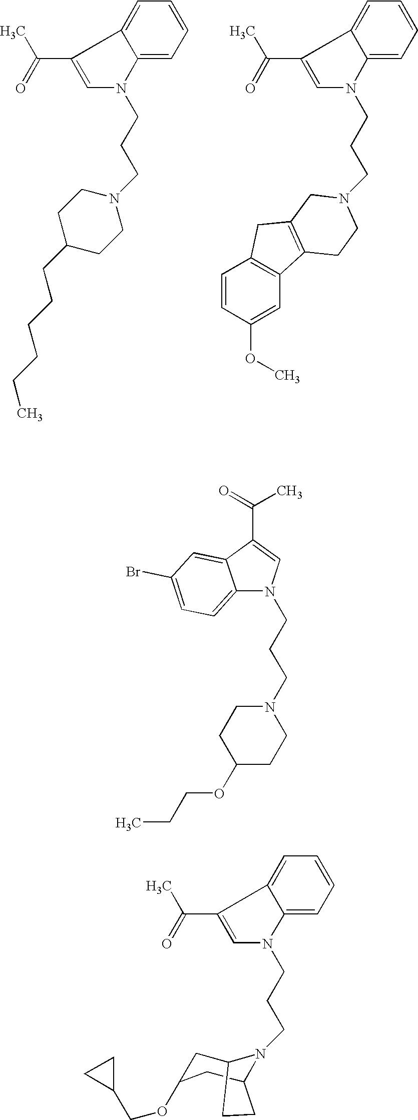

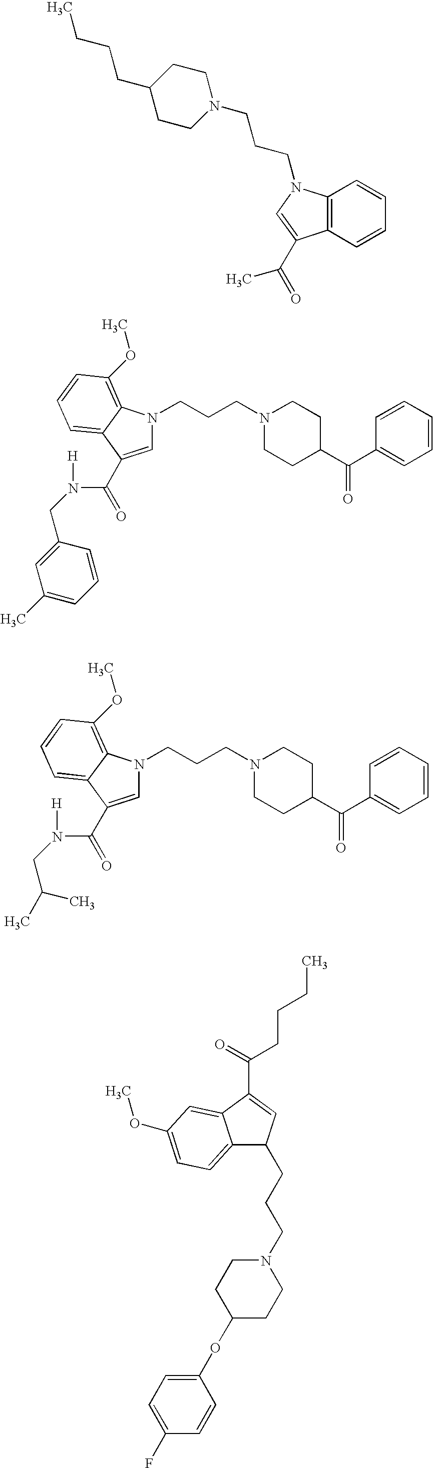

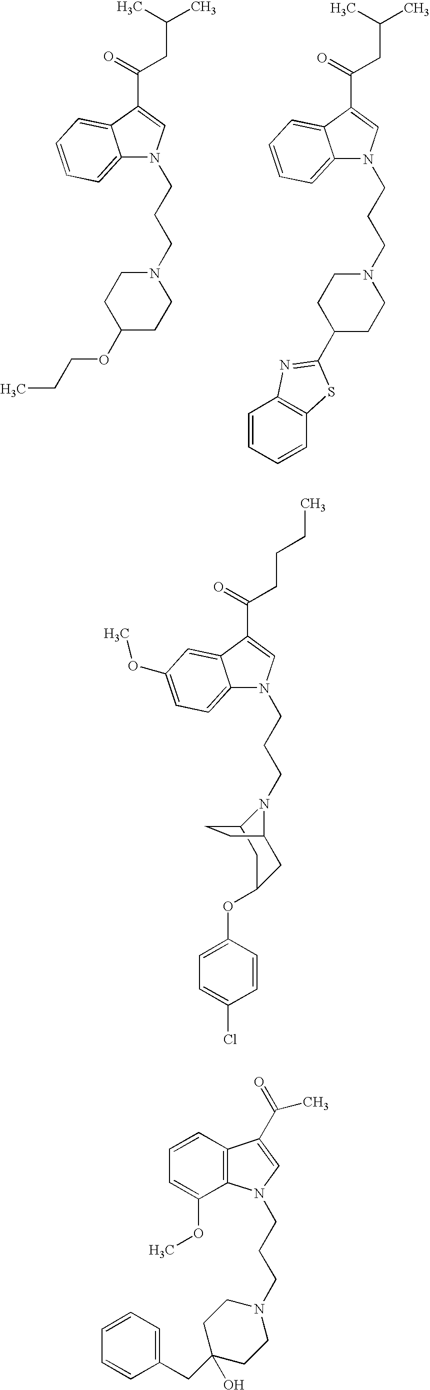

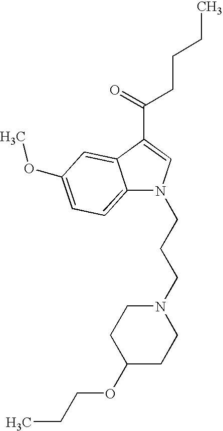

71. The compound of claim 1, wherein the compound is selected from the group consisting of:

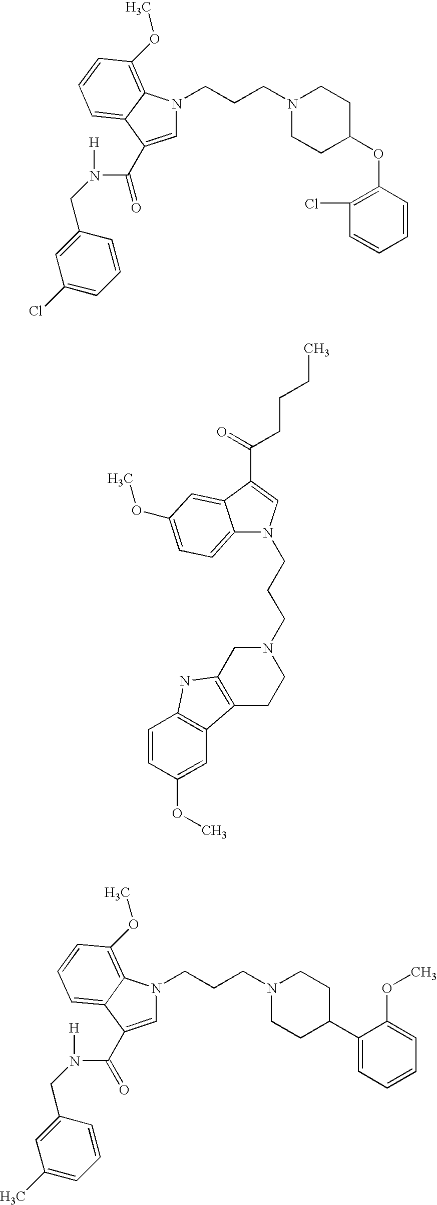

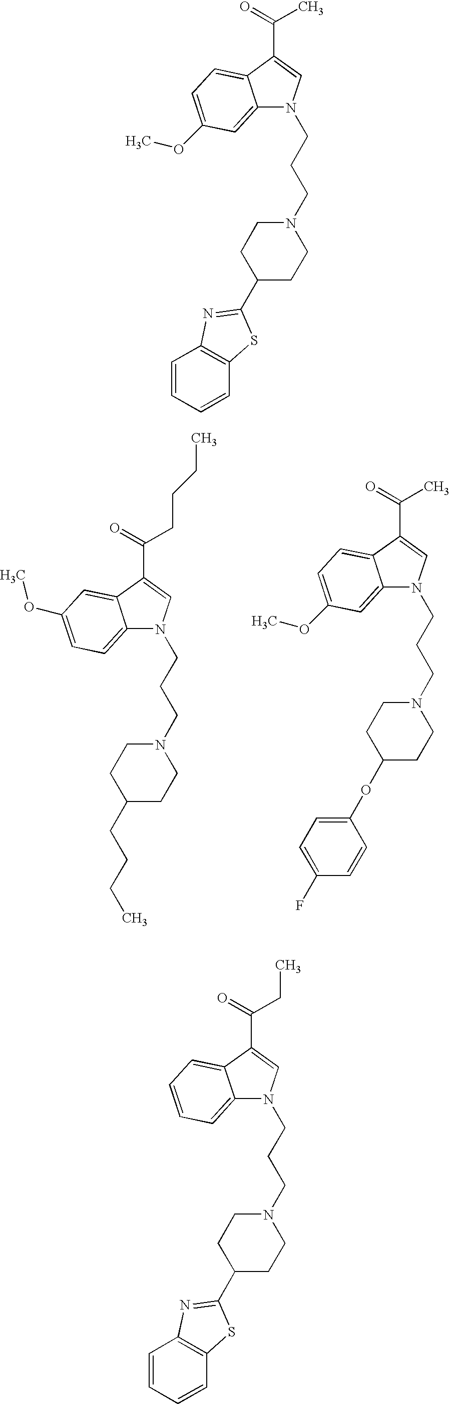

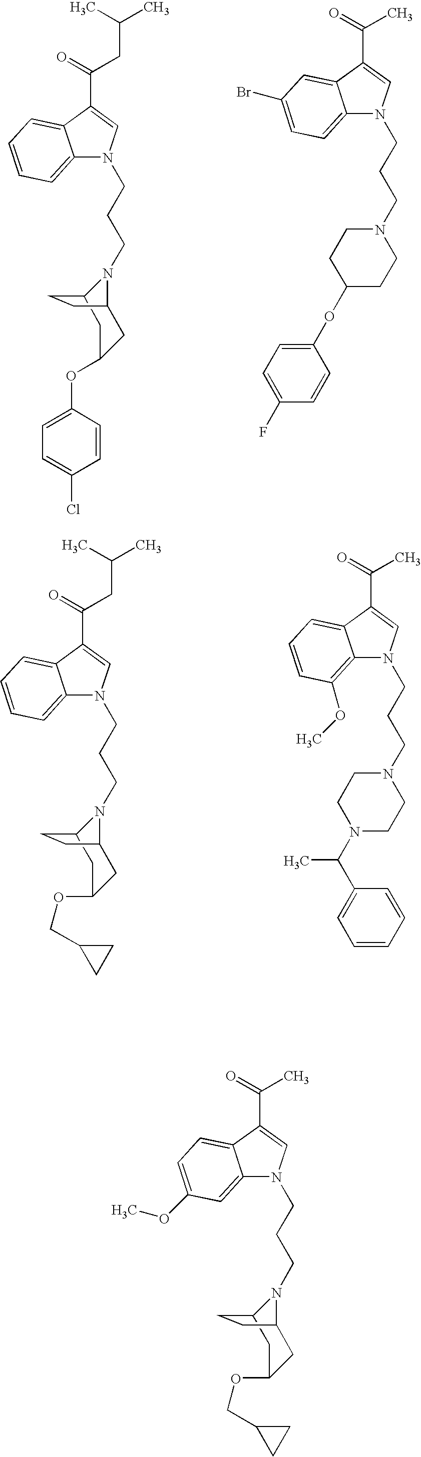

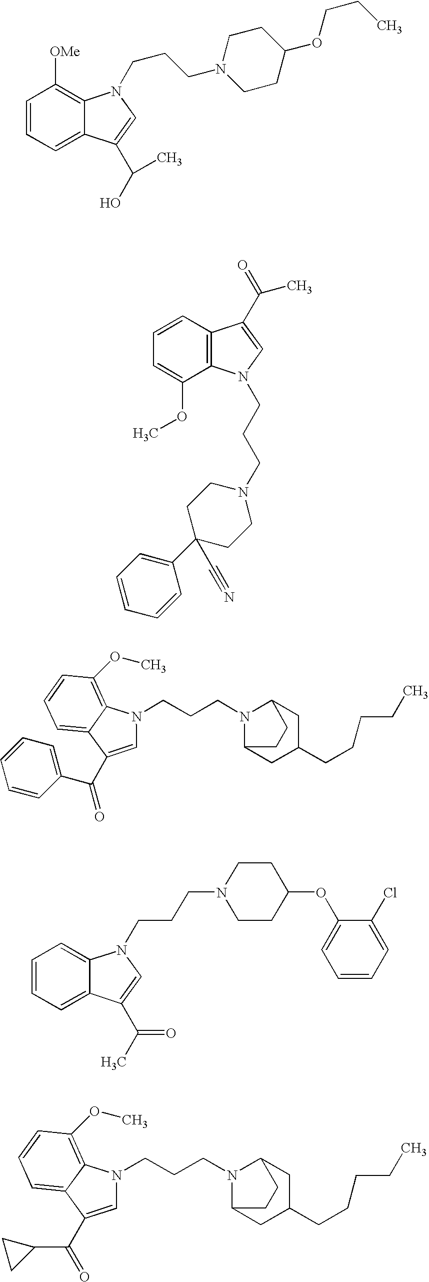

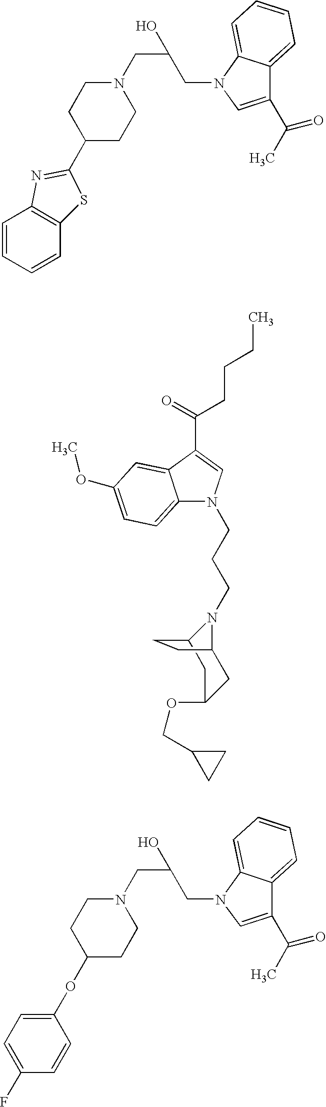

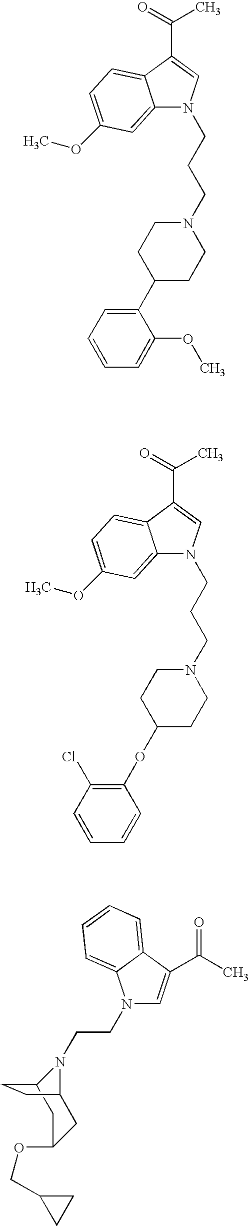

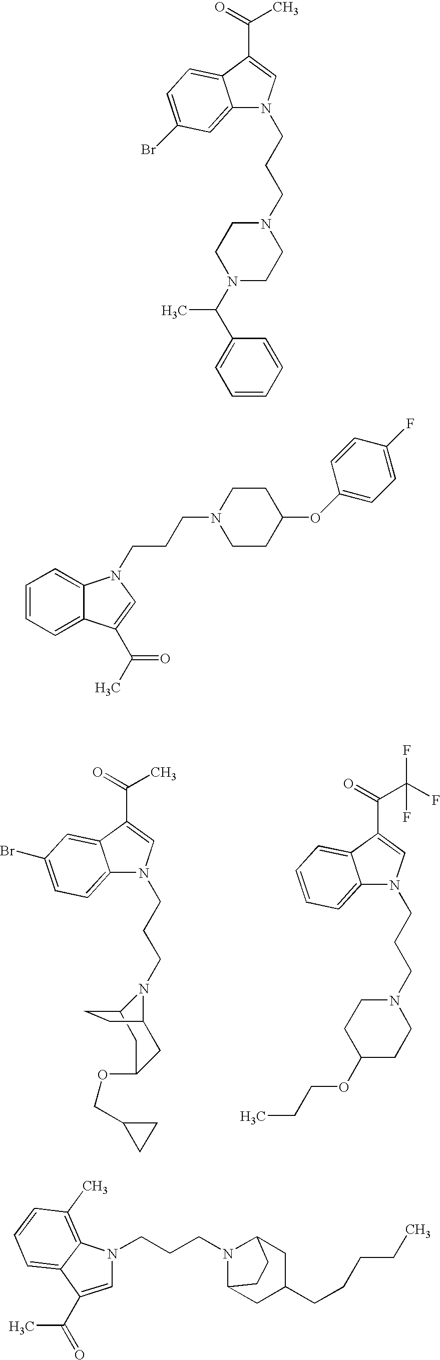

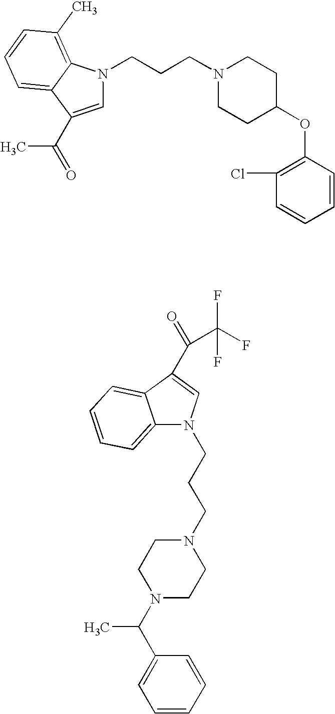

72. The compound of claim 1, wherein the compound is selected from the group consisting of:

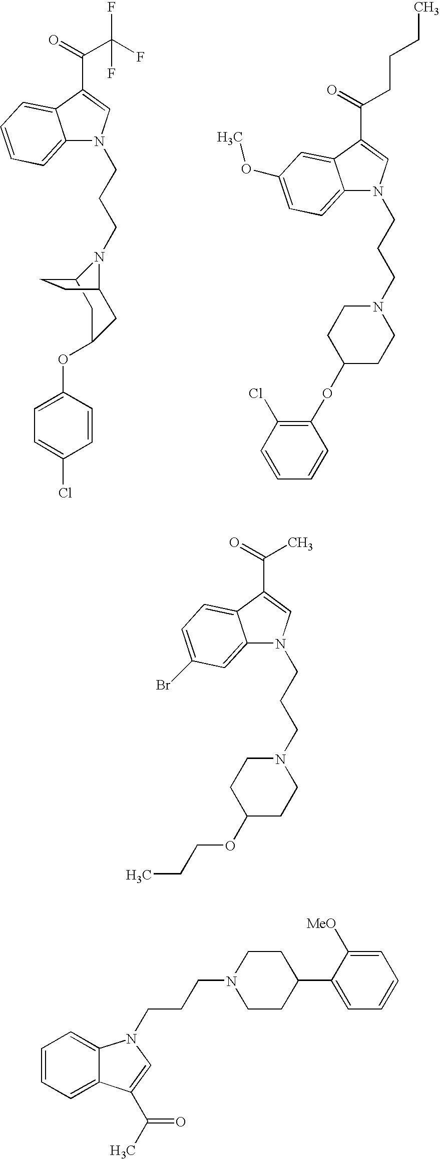

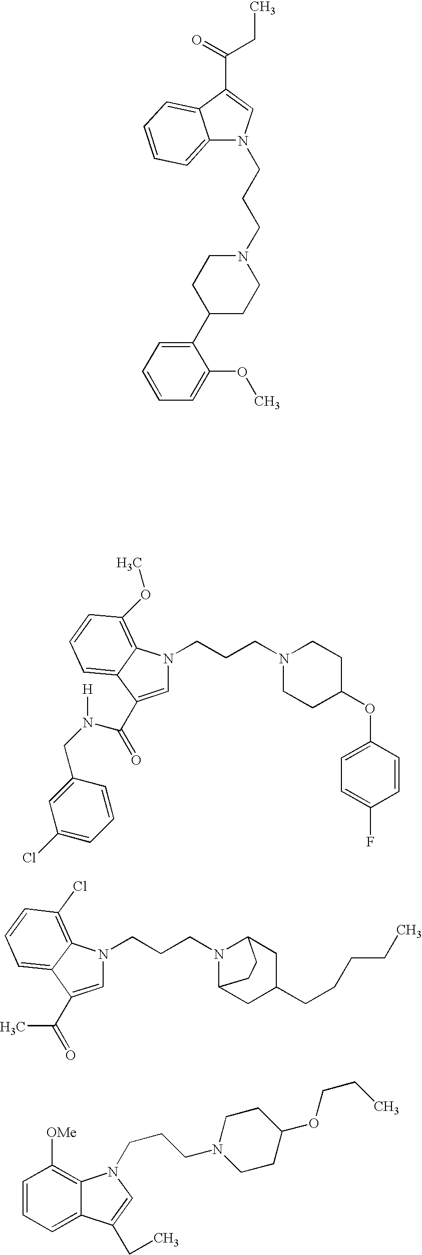

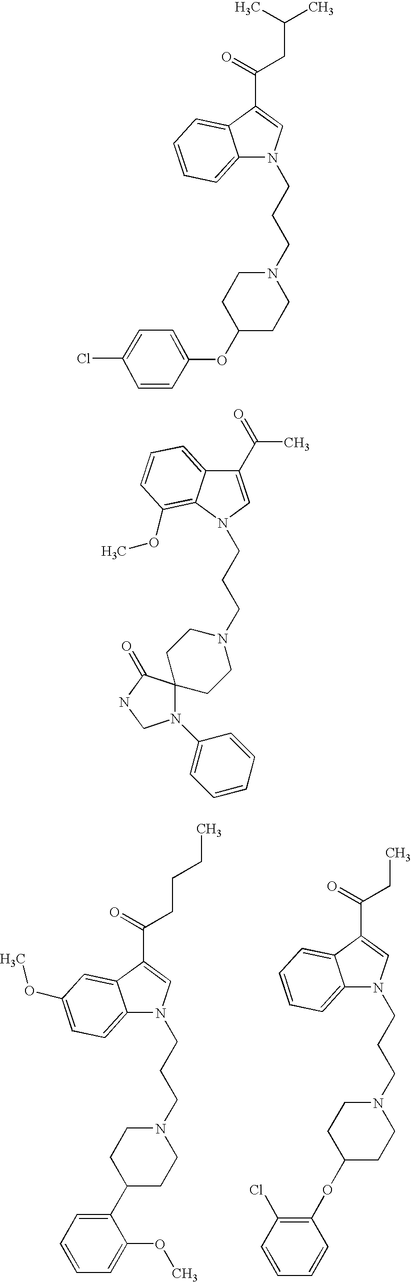

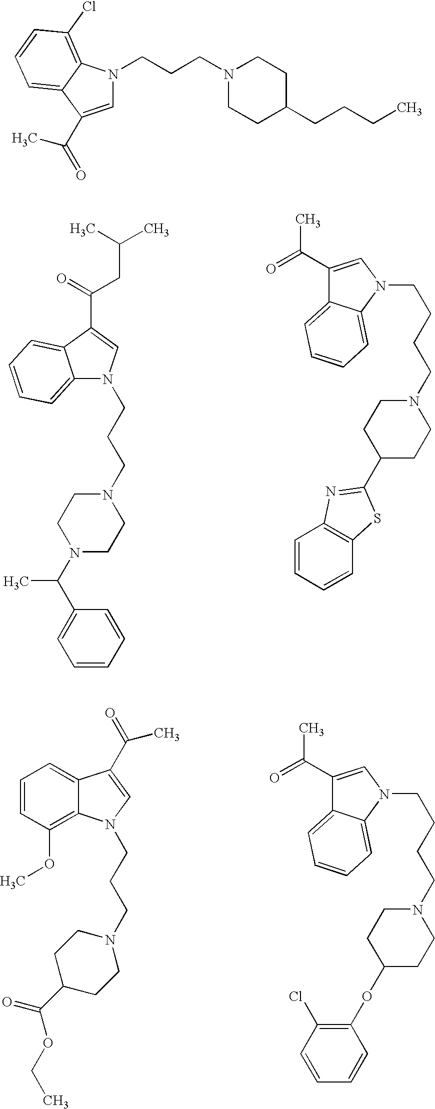

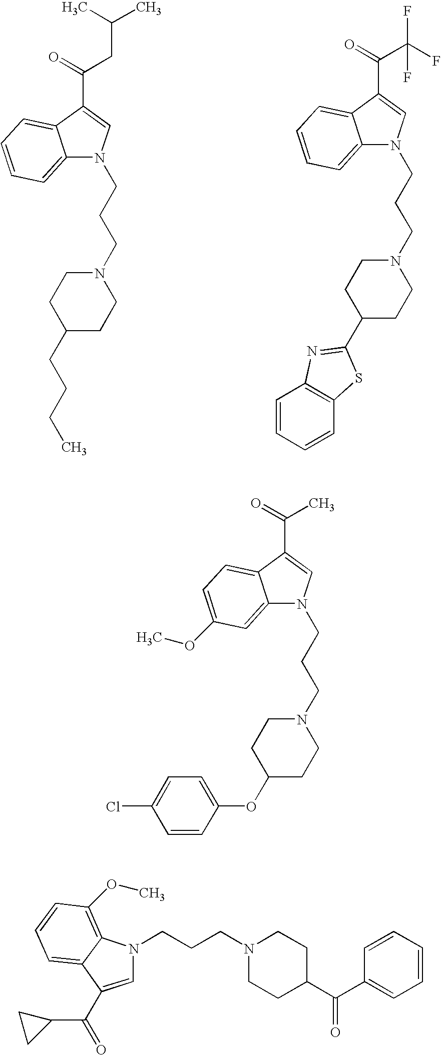

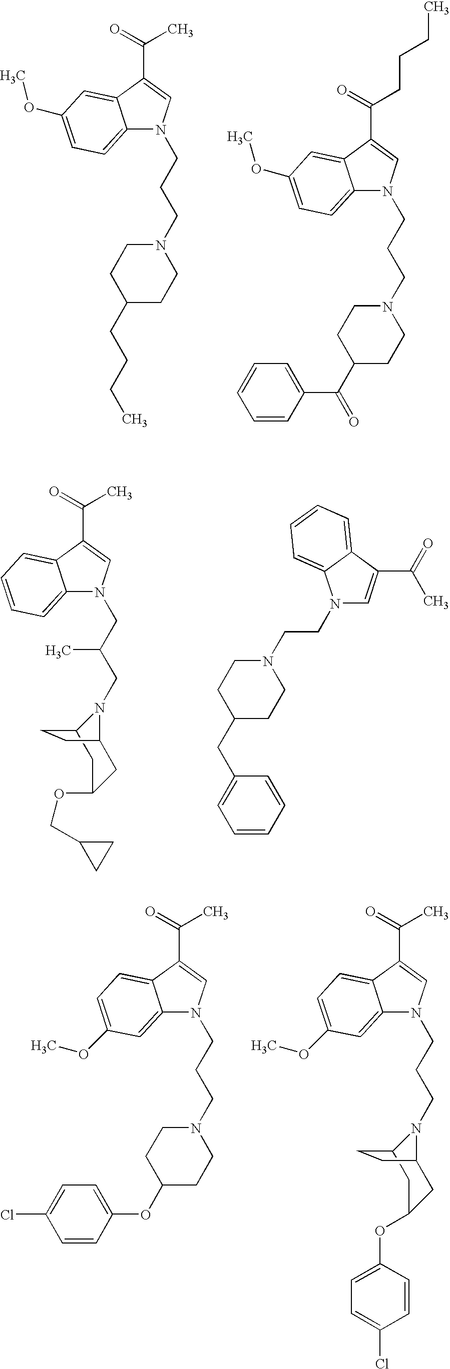

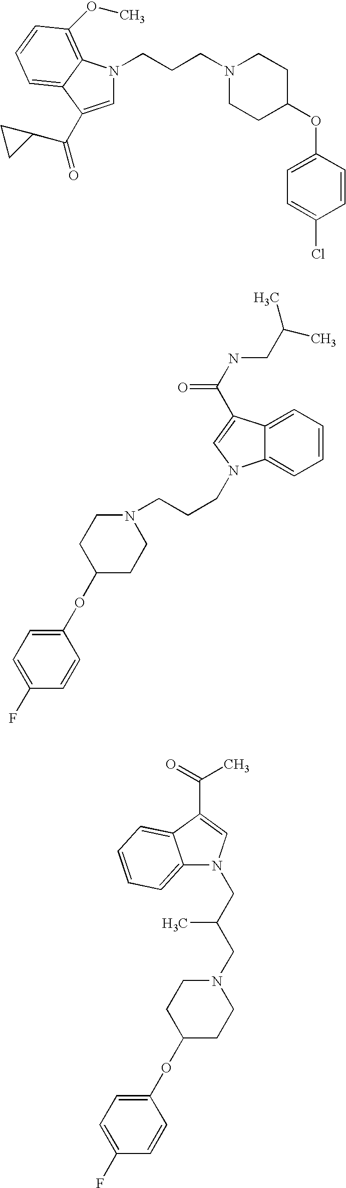

73. The compound of claim 1, wherein the compound is selected from the group consisting of:

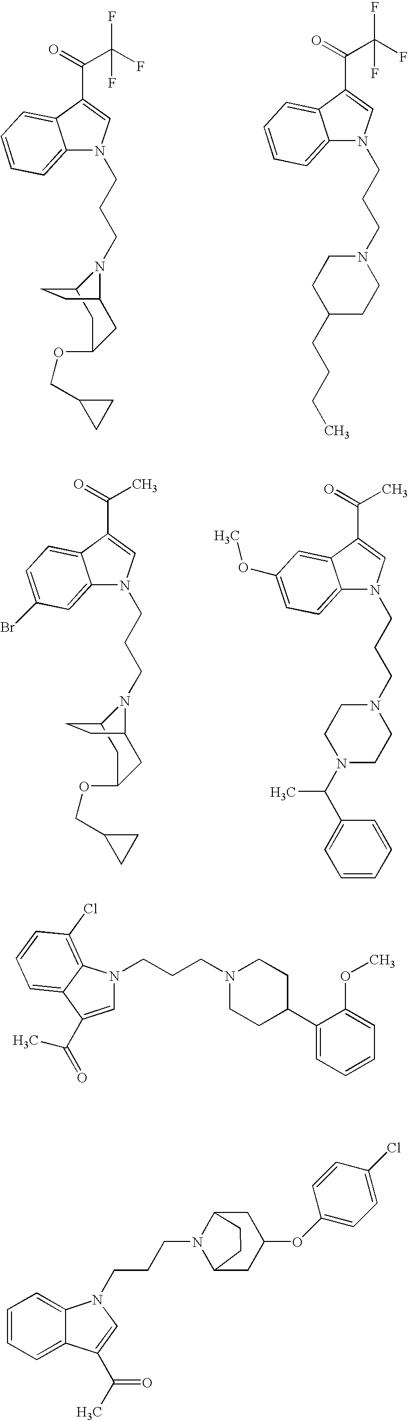

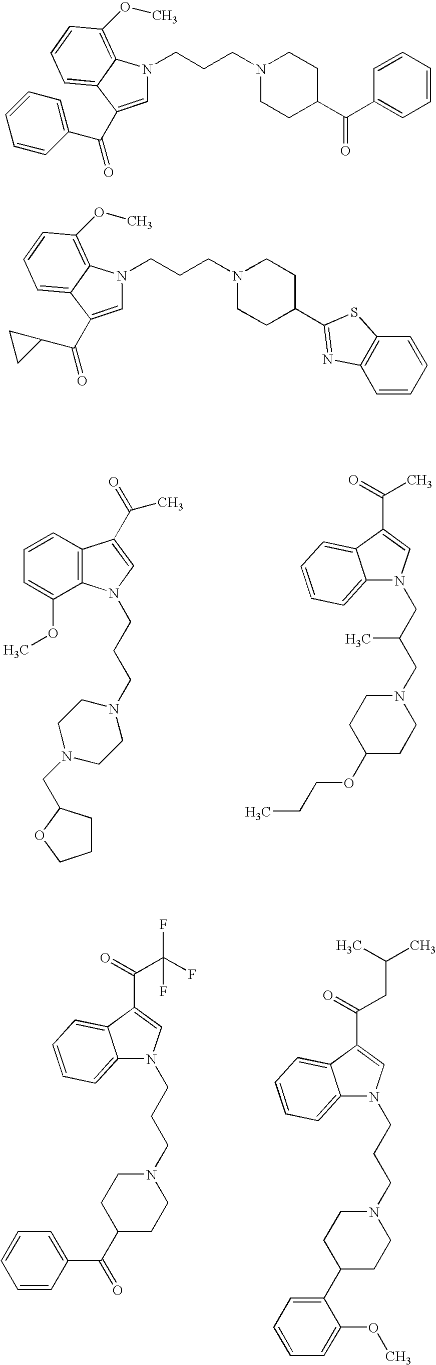

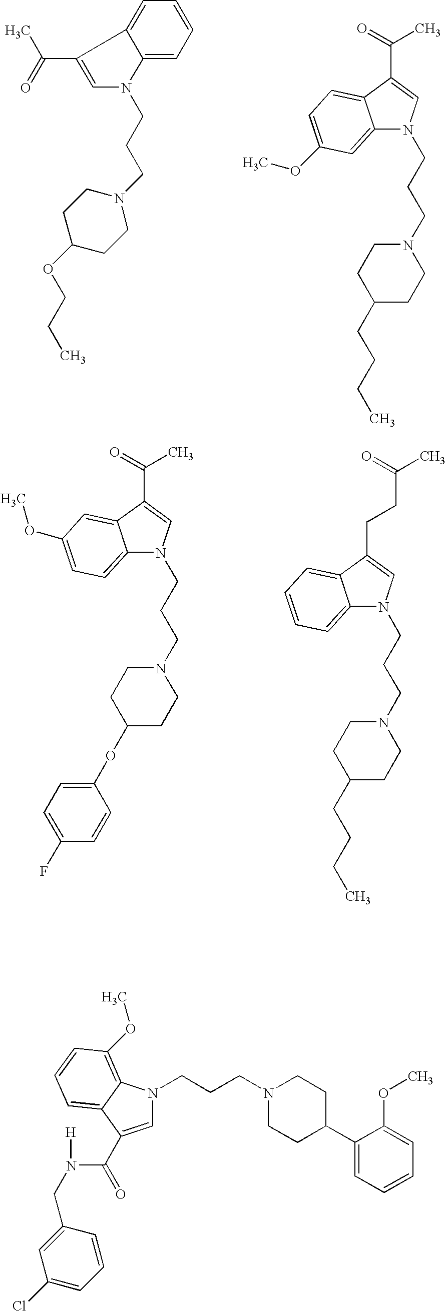

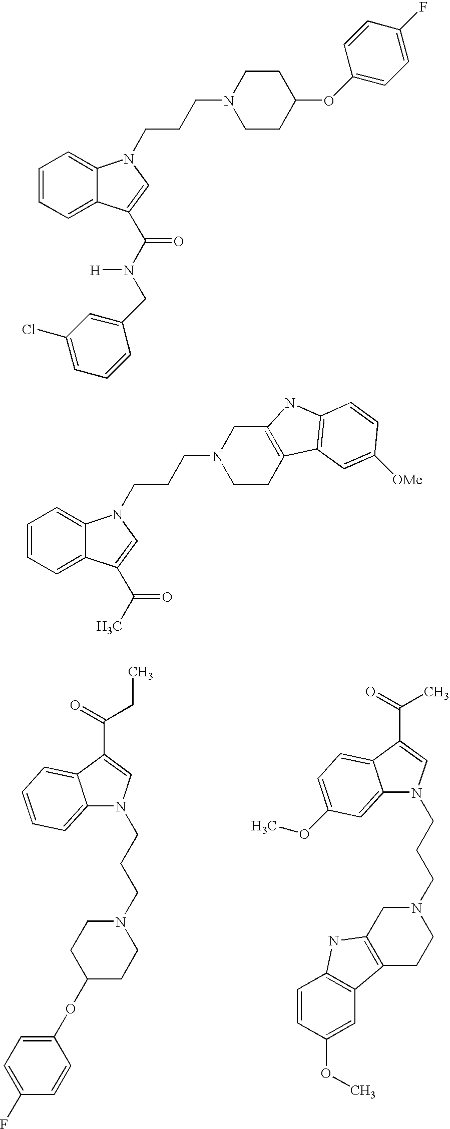

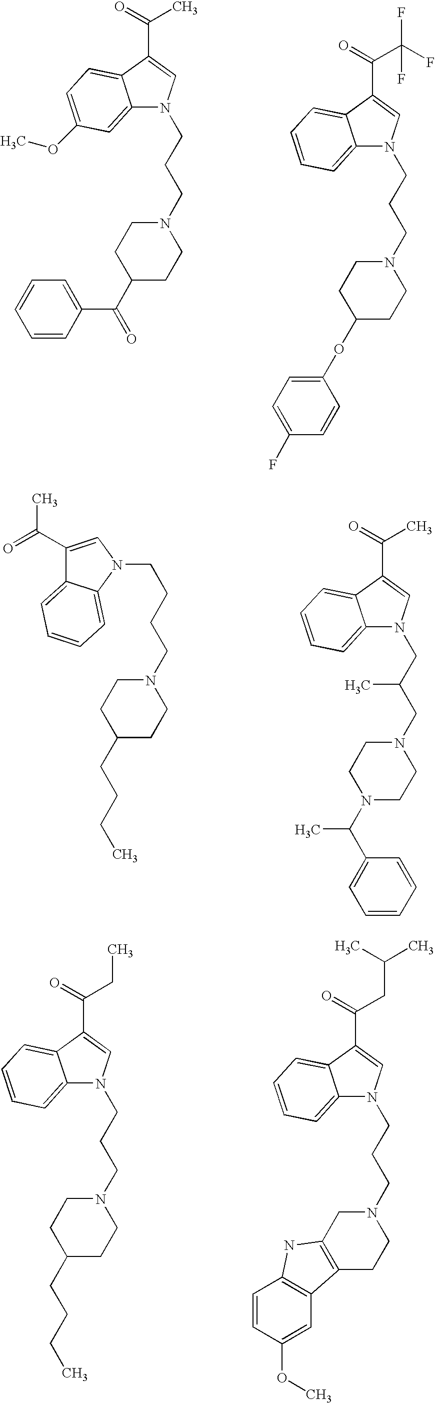

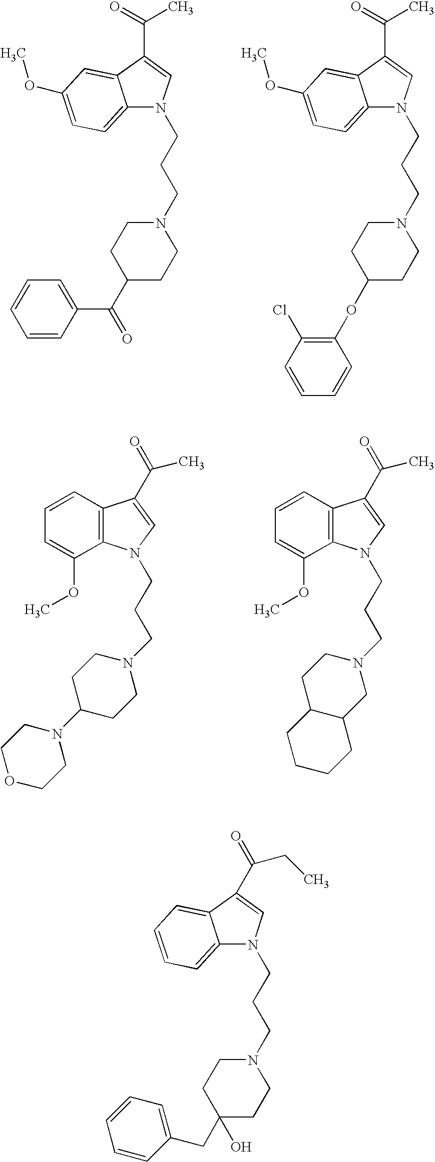

74. The compound of claim 1, wherein the compound is selected from the group consisting of:

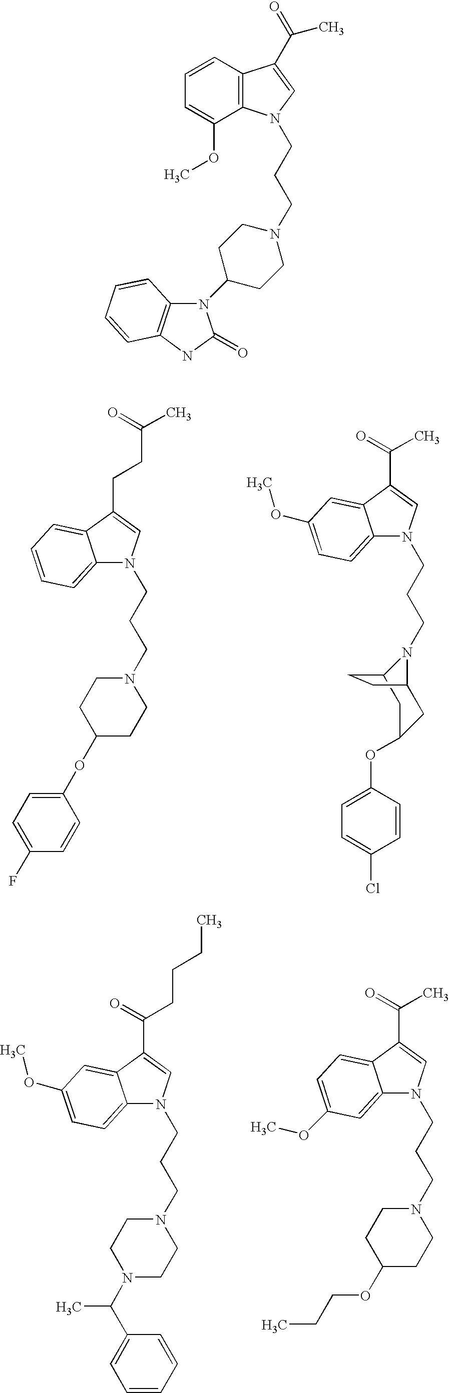

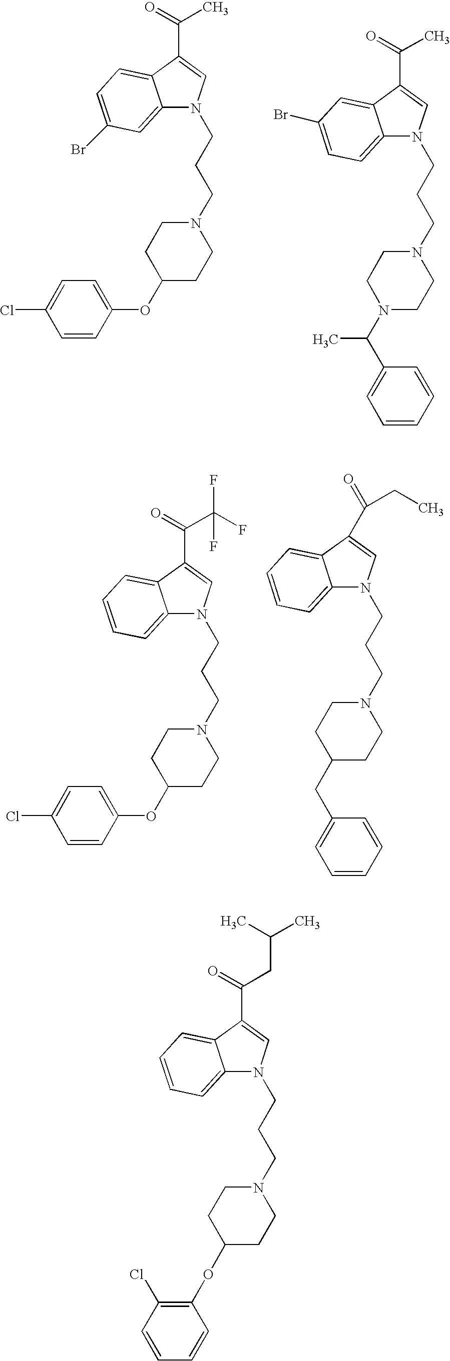

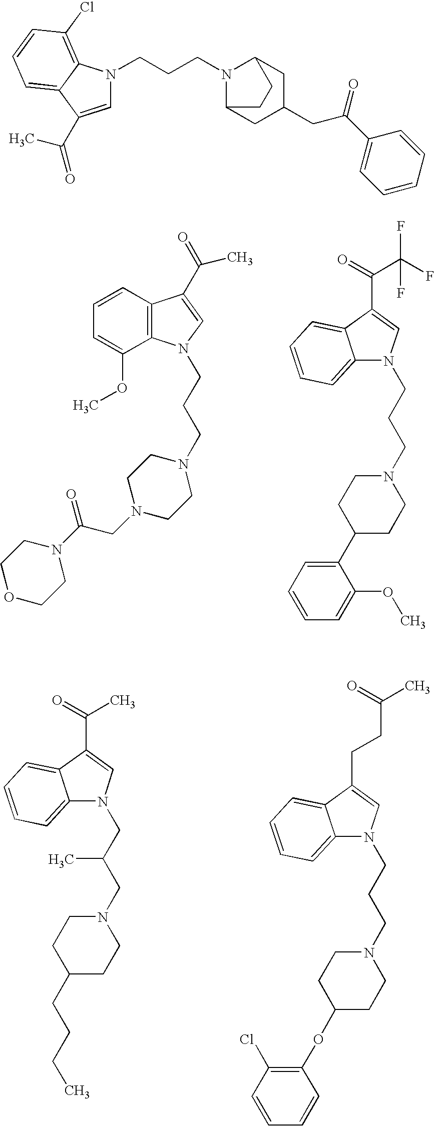

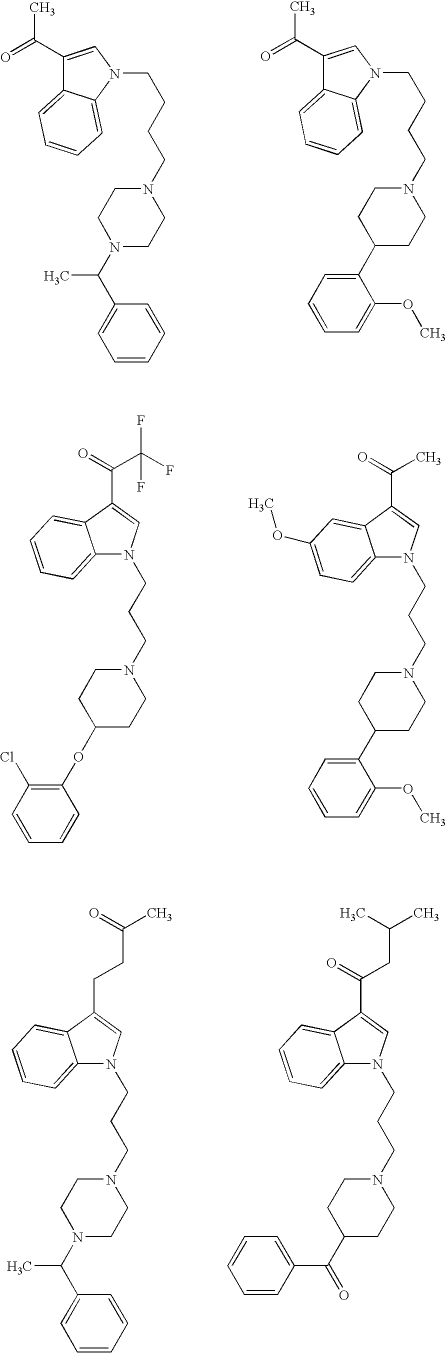

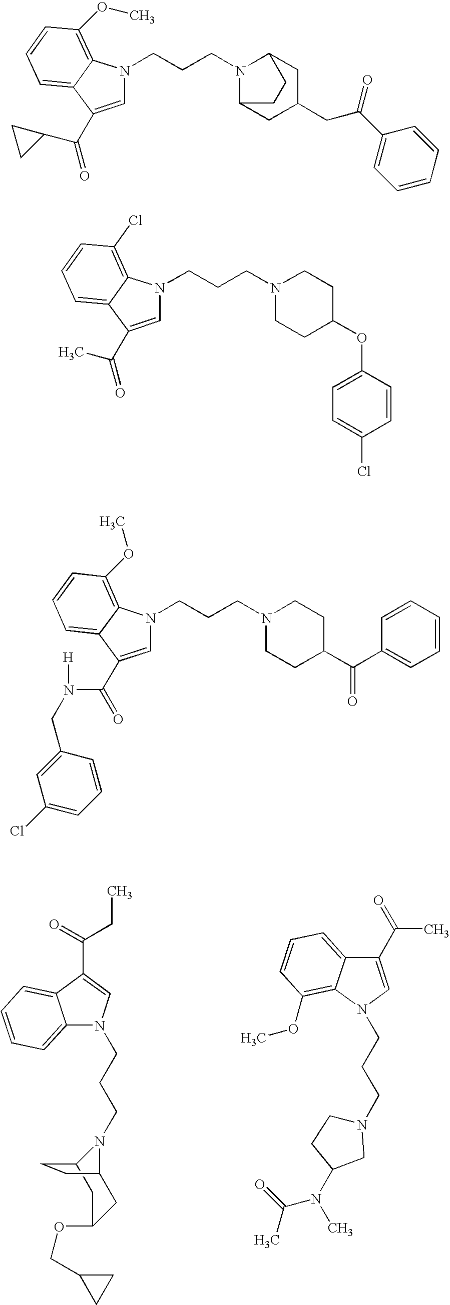

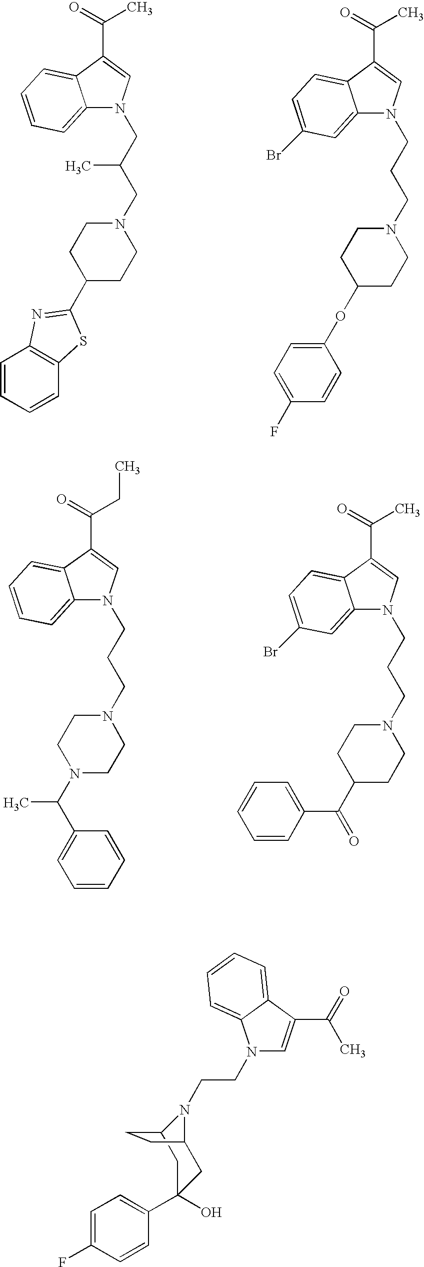

75. The compound of claim 1, wherein the compound is selected from the group consisting of:

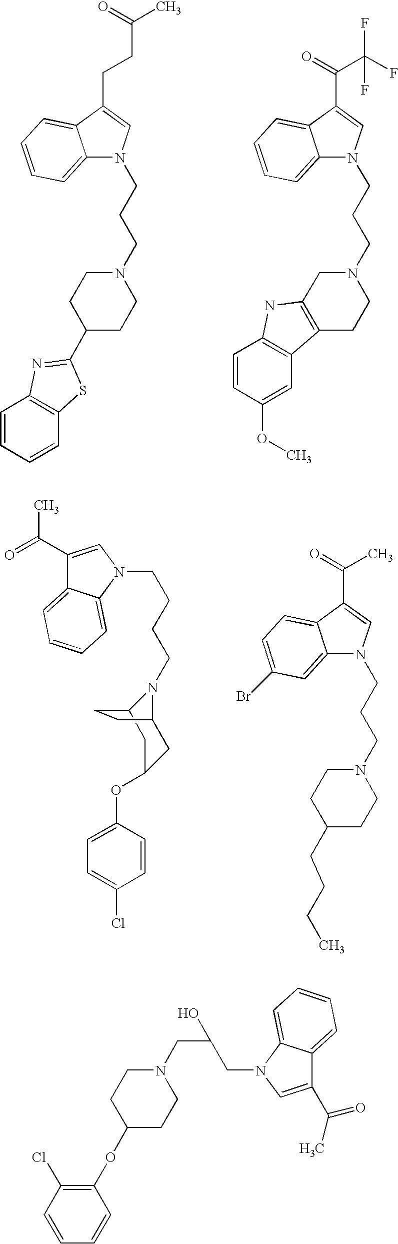

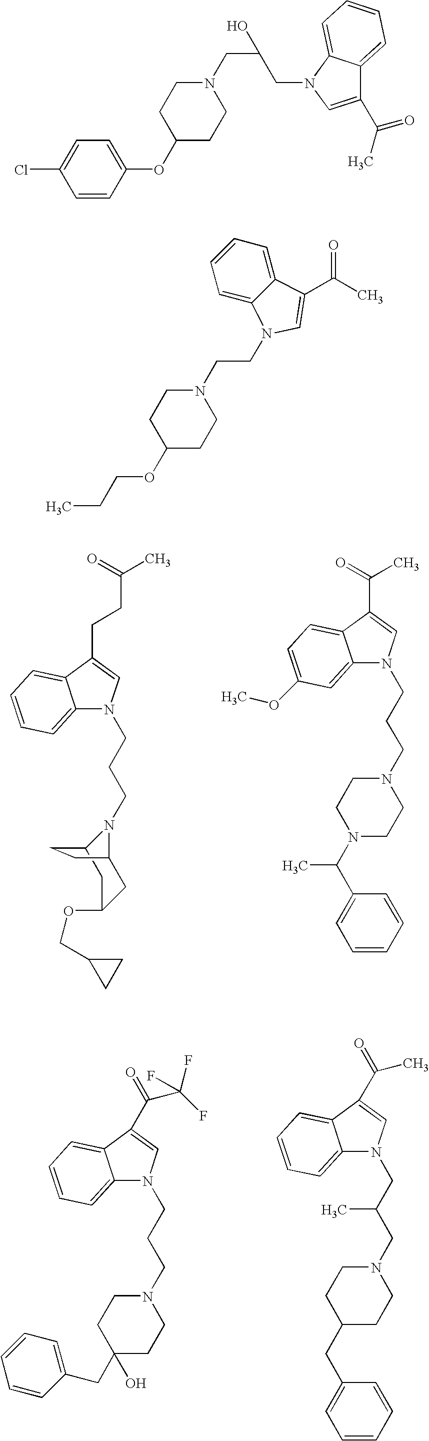

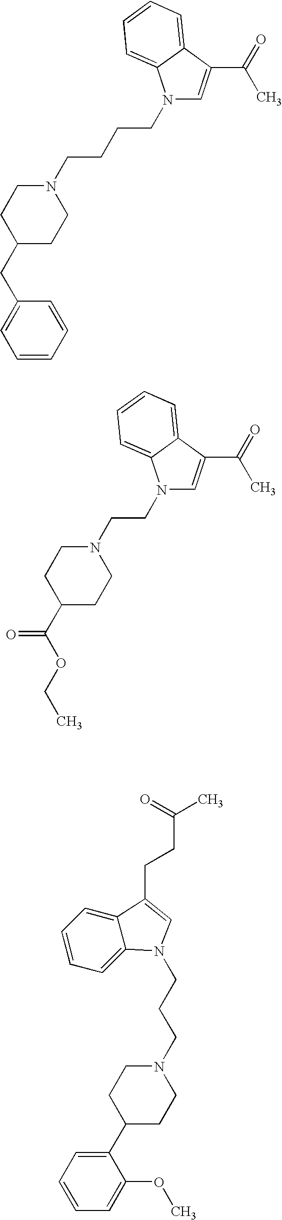

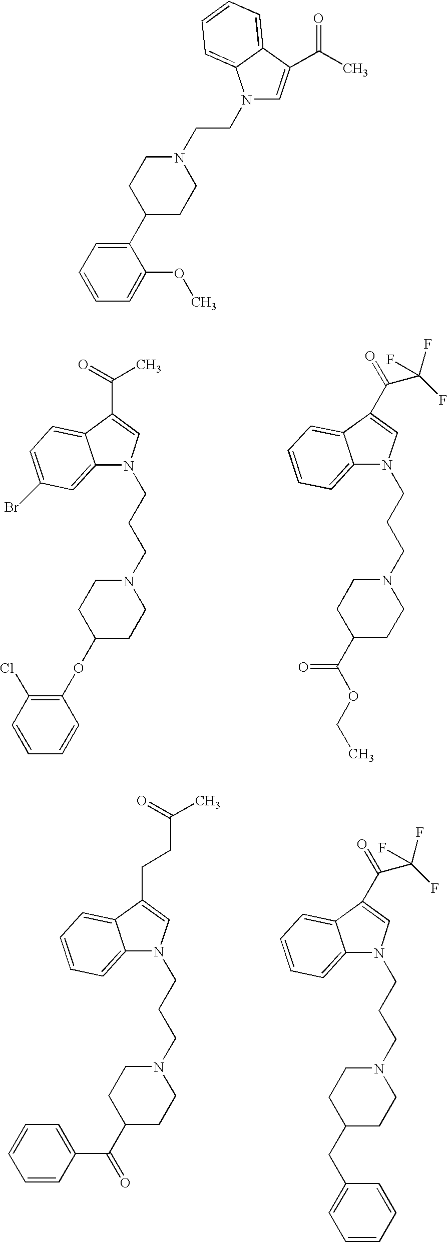

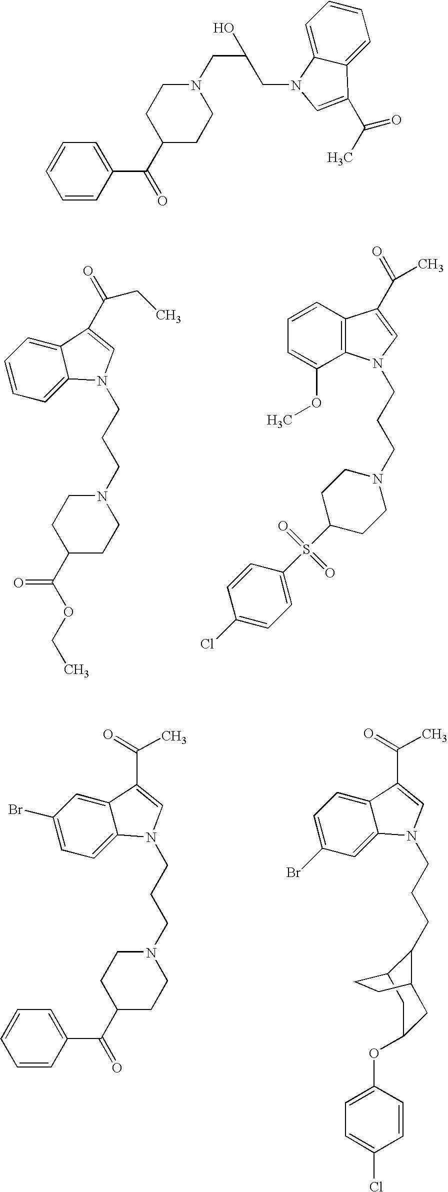

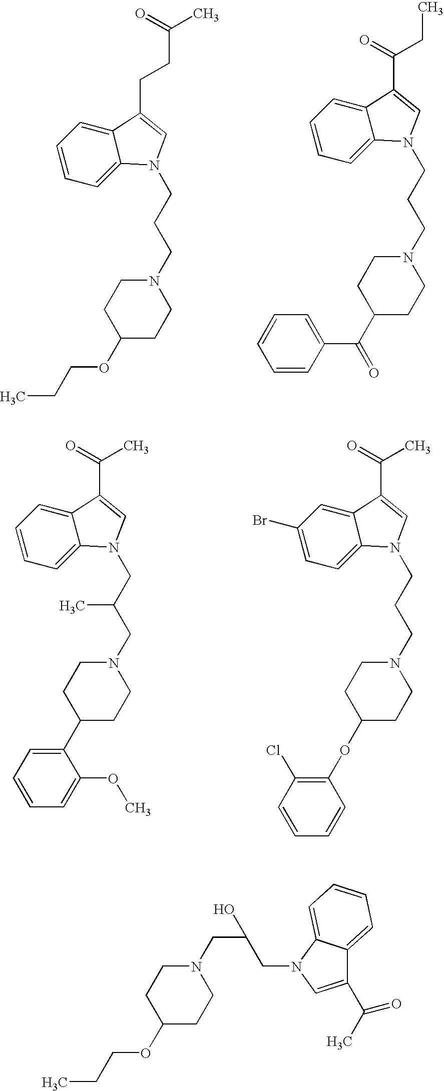

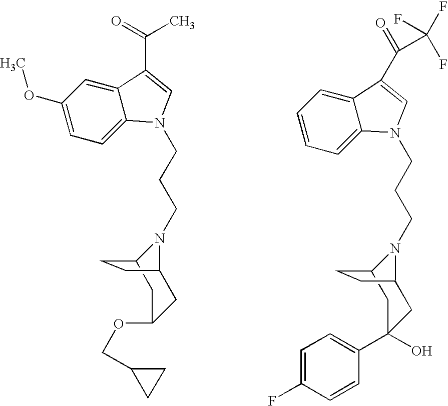

76. The compound of claim 1, wherein the compound is selected from the group consisting of:

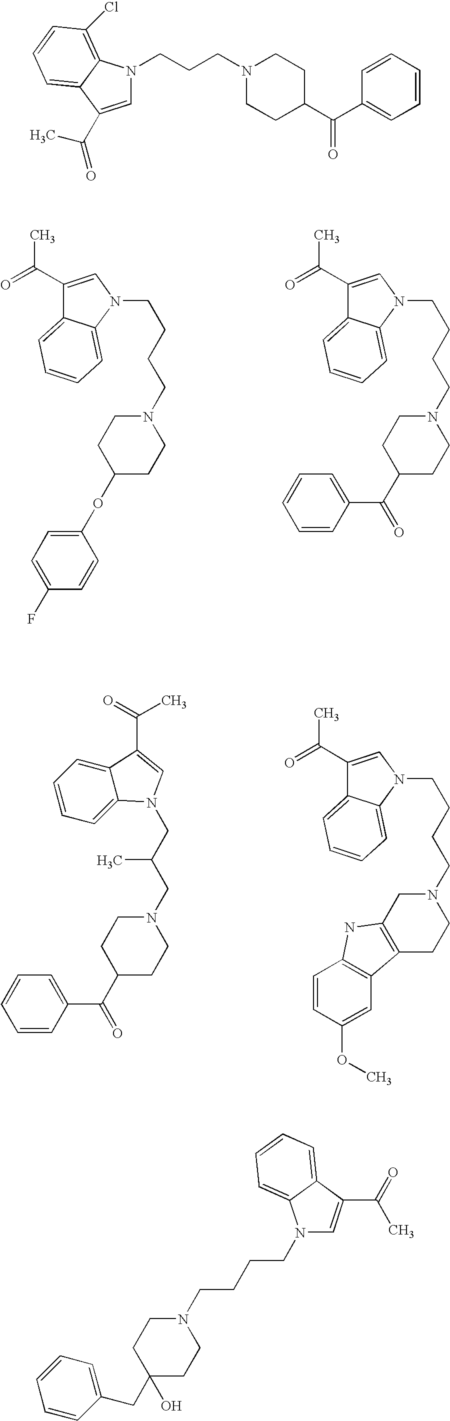

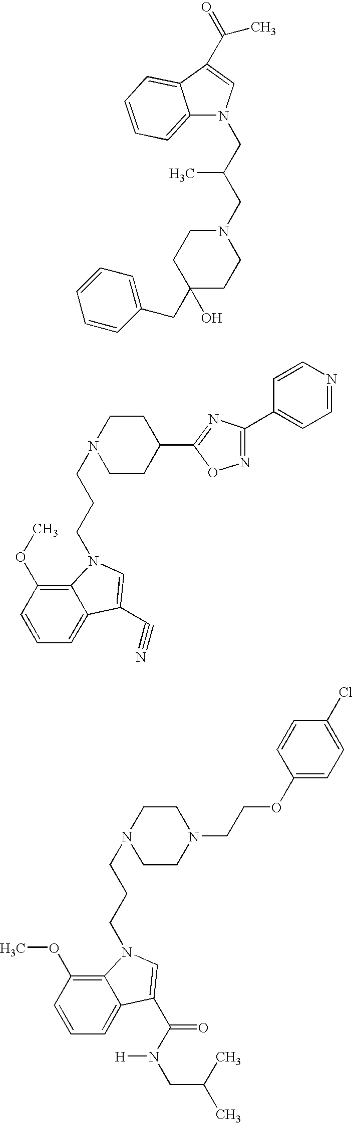

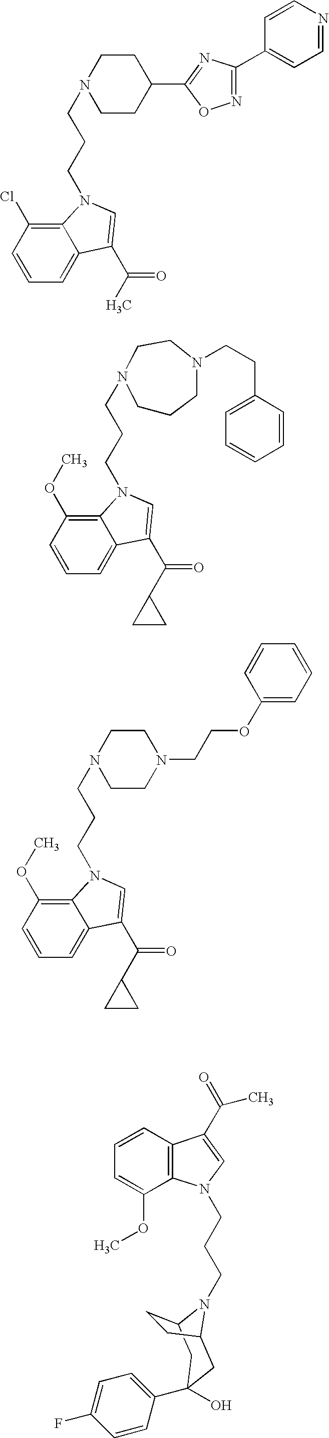

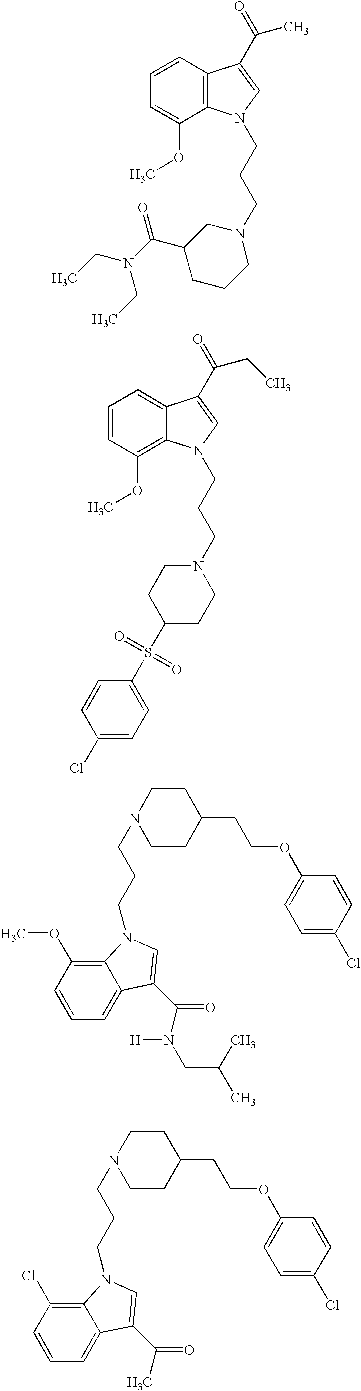

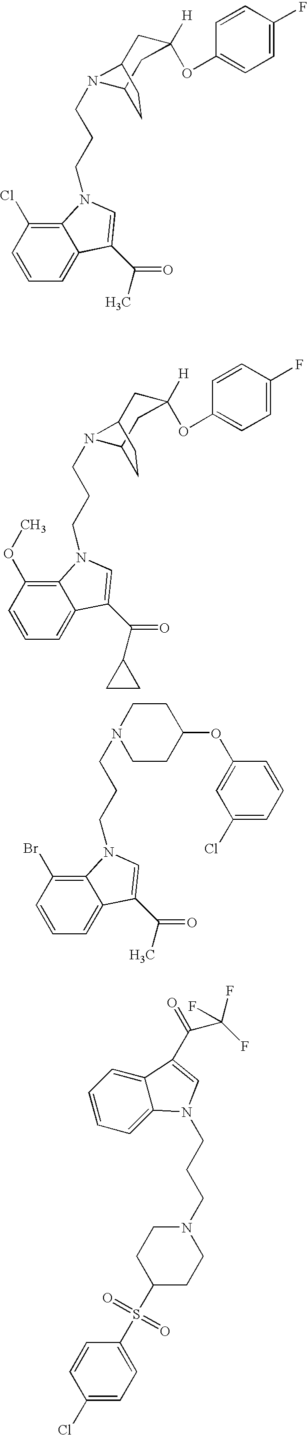

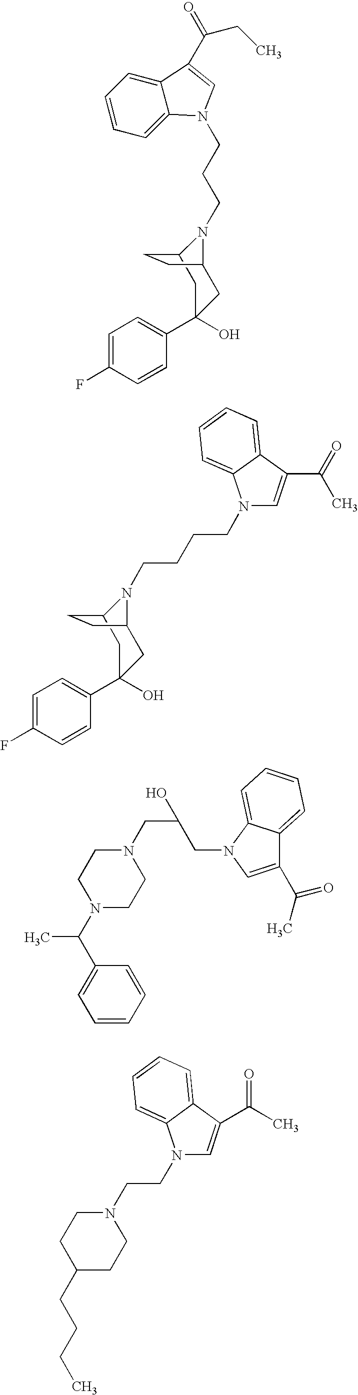

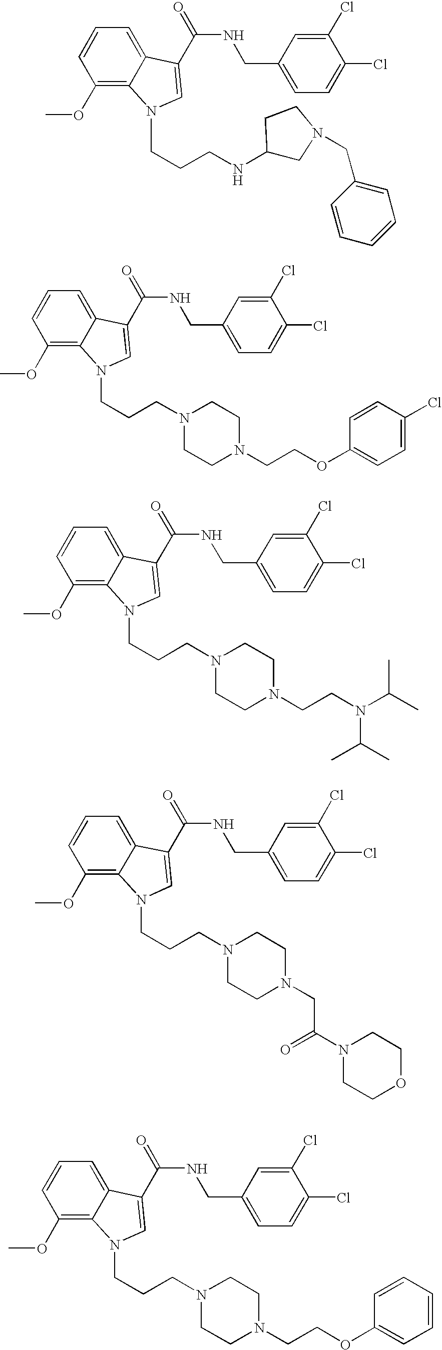

77. The compound of claim 1, wherein the compound is selected from the group consisting of:

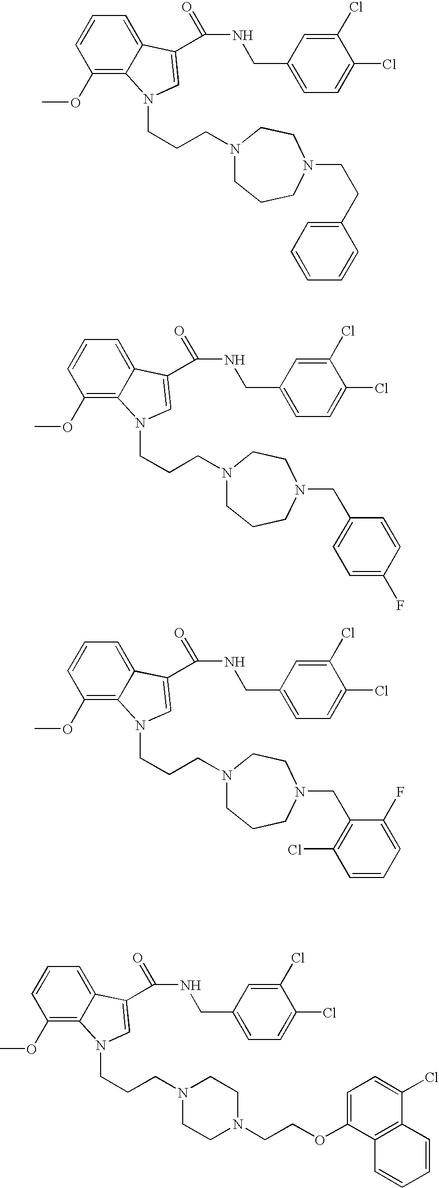

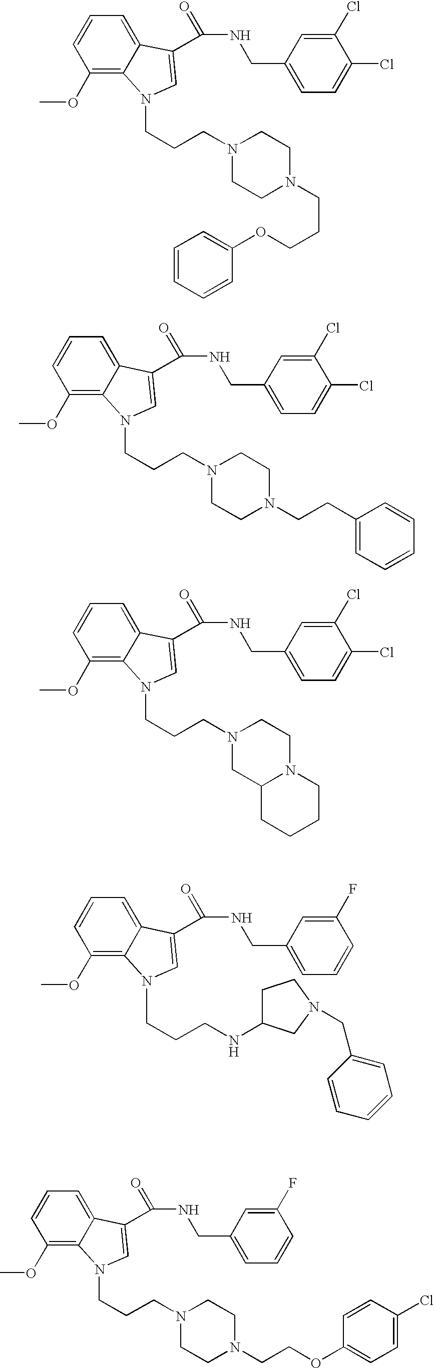

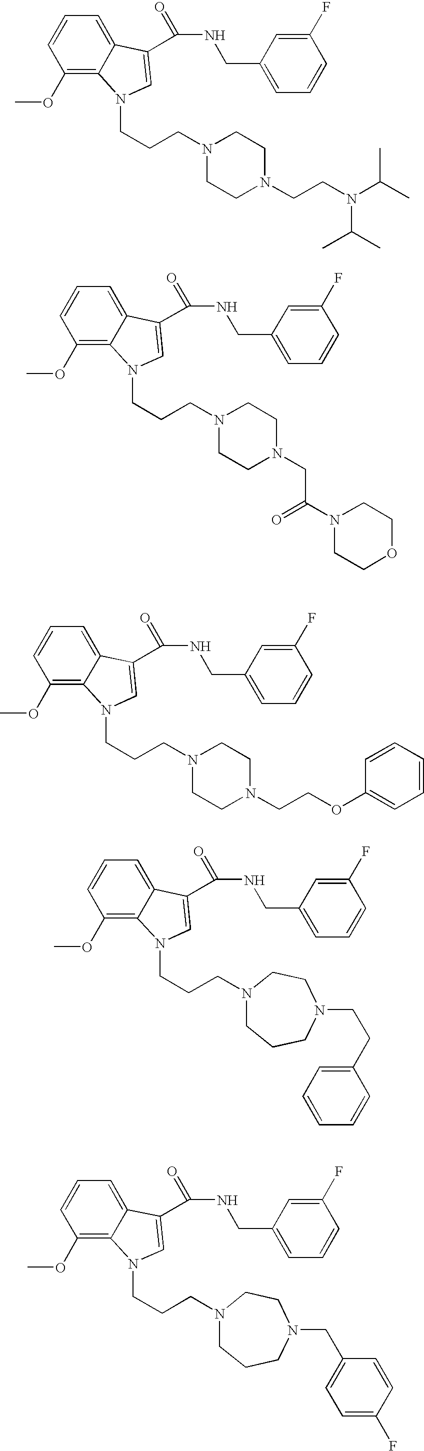

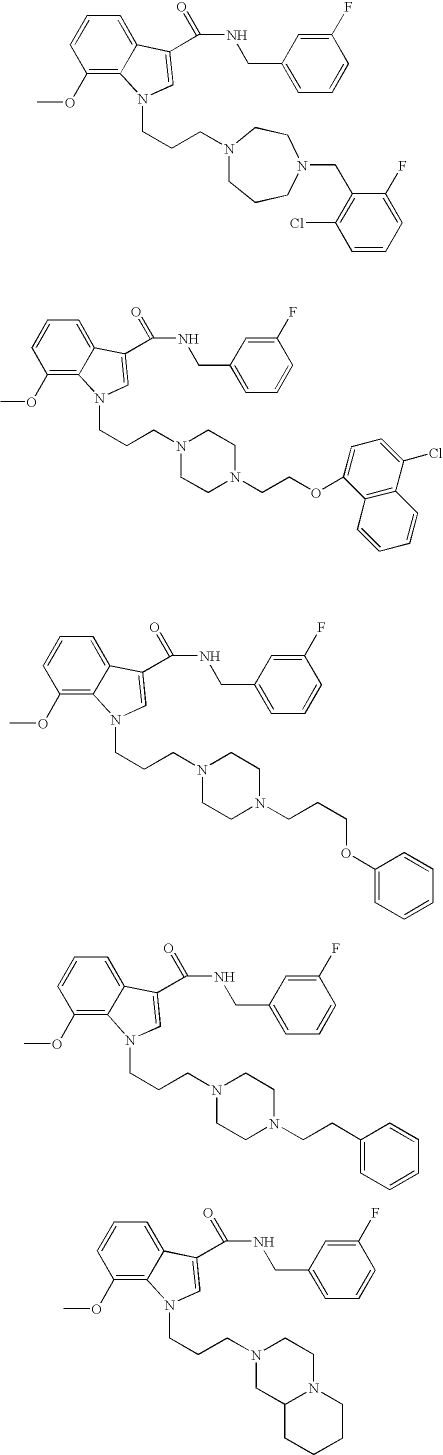

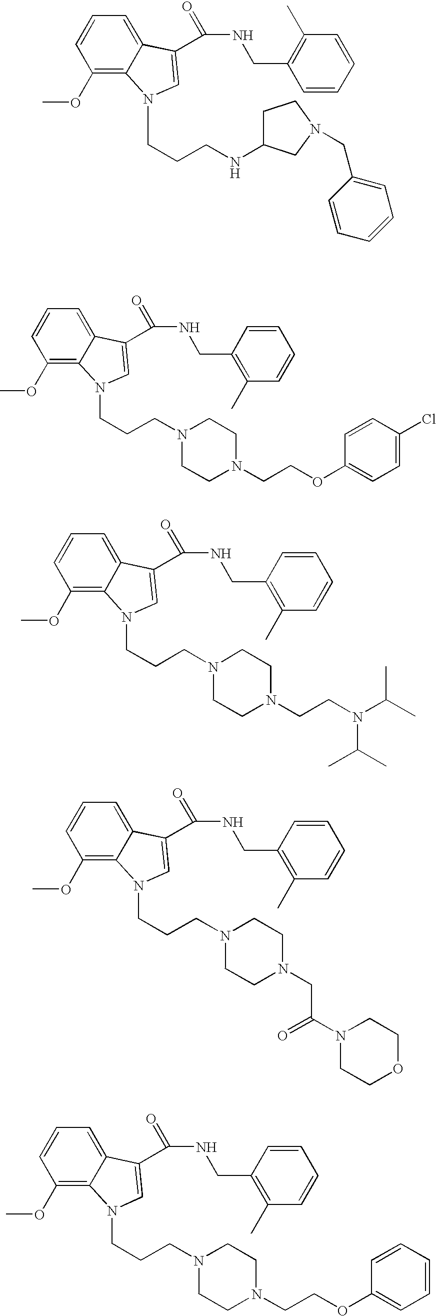

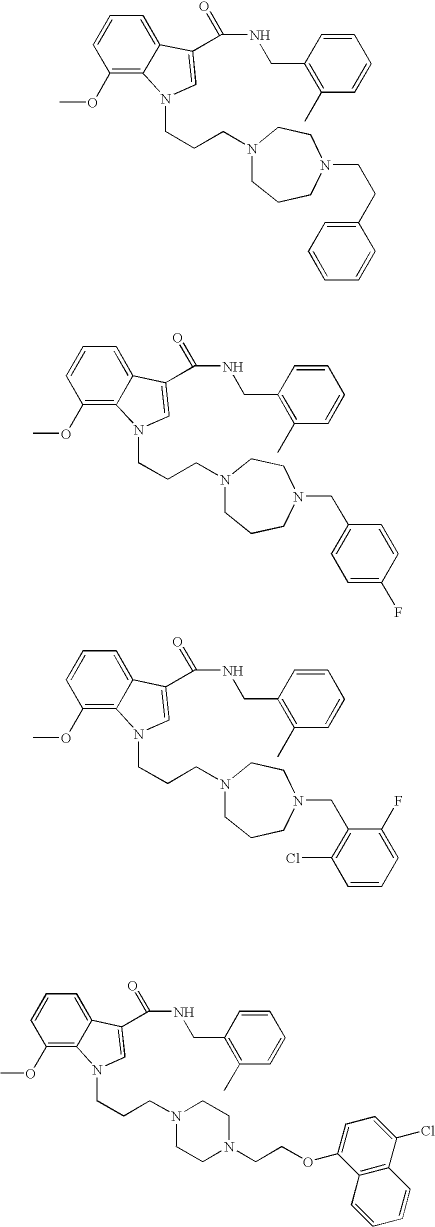

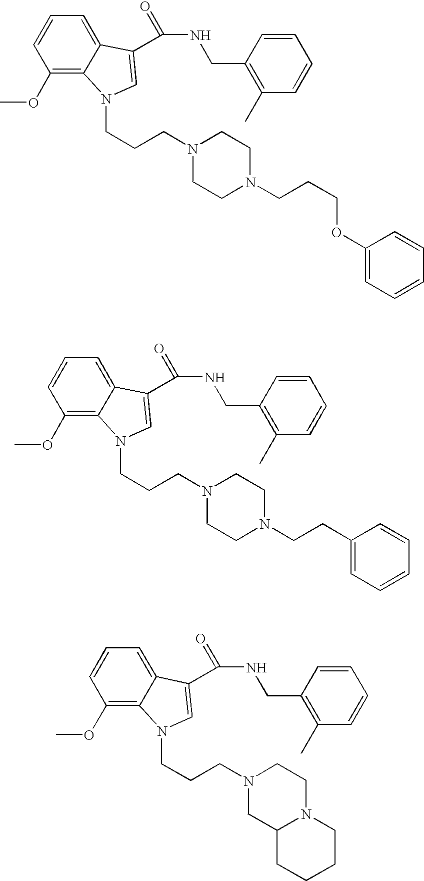

78. The compound of claim 1, wherein the compound is selected from the group consisting of:

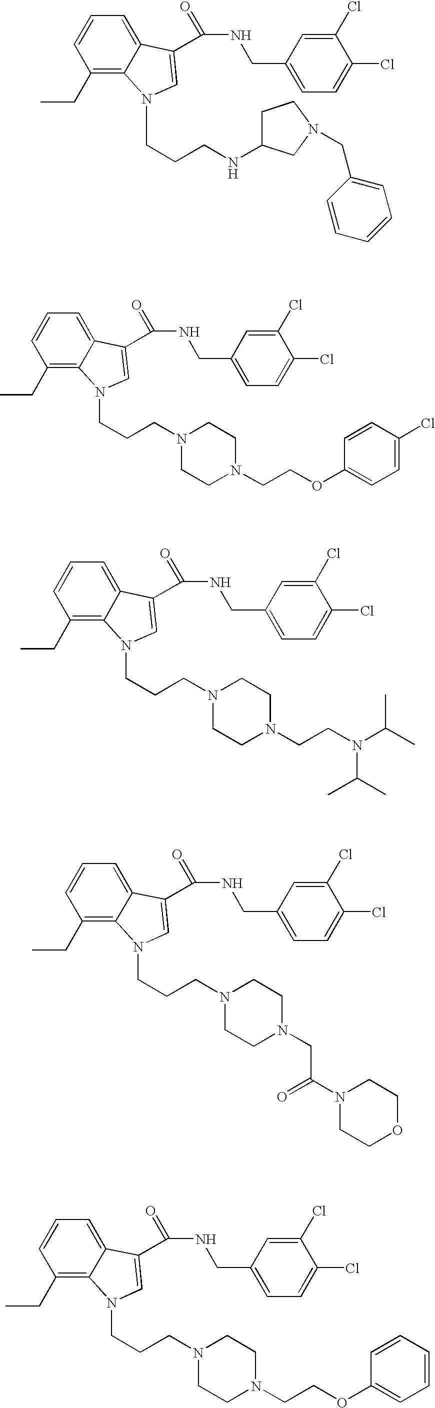

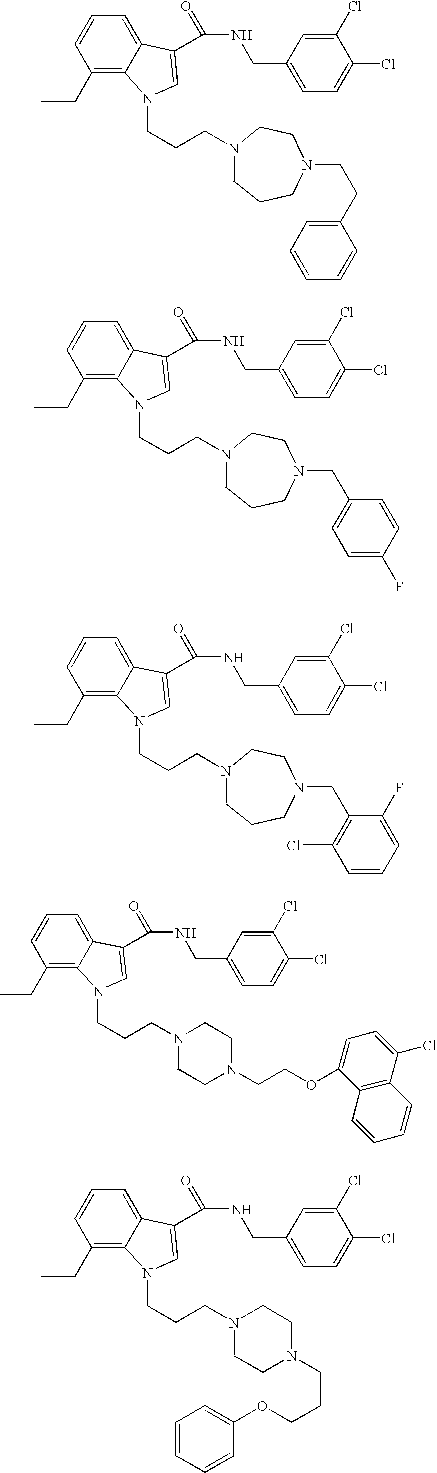

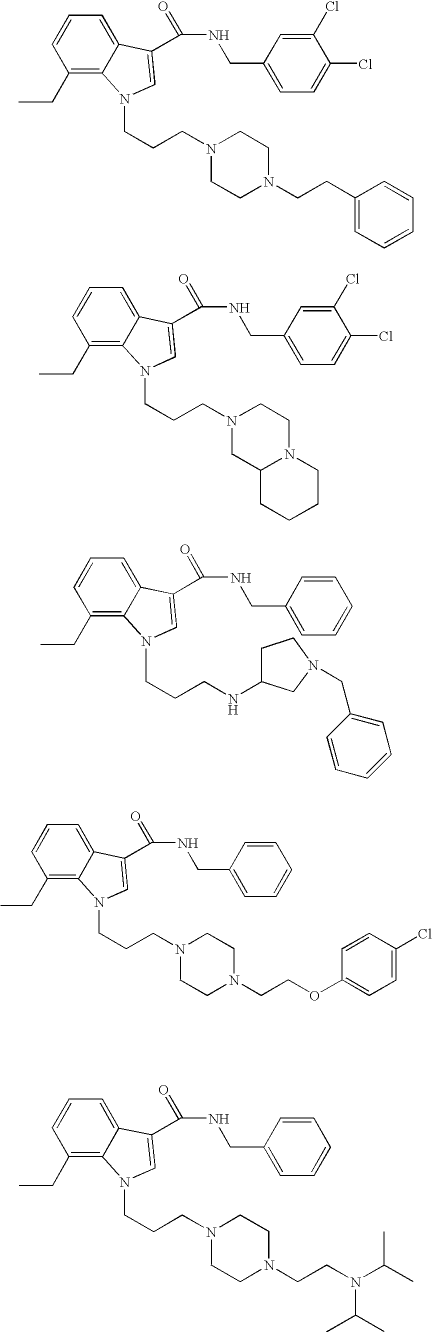

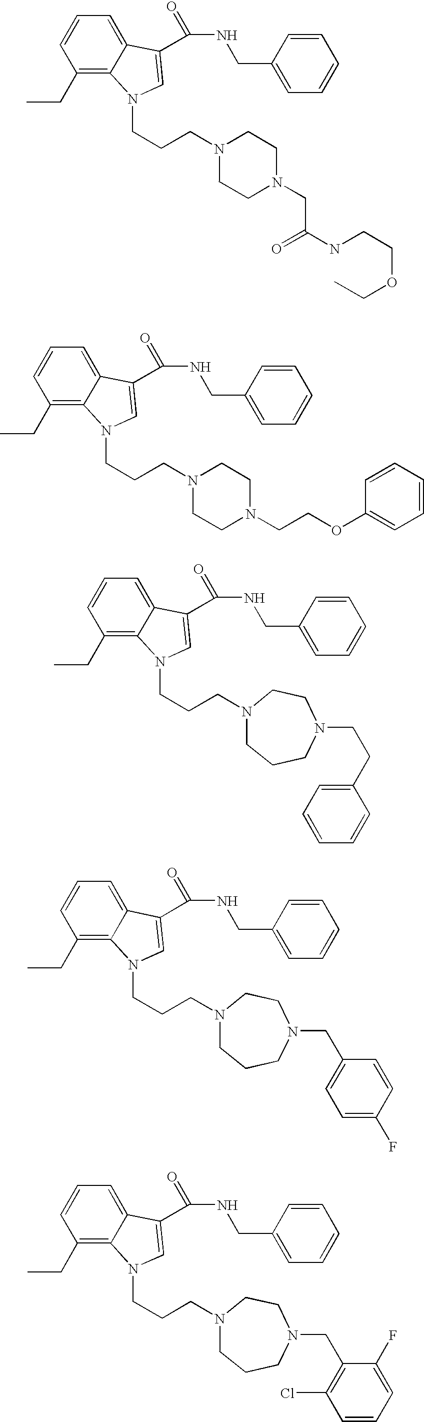

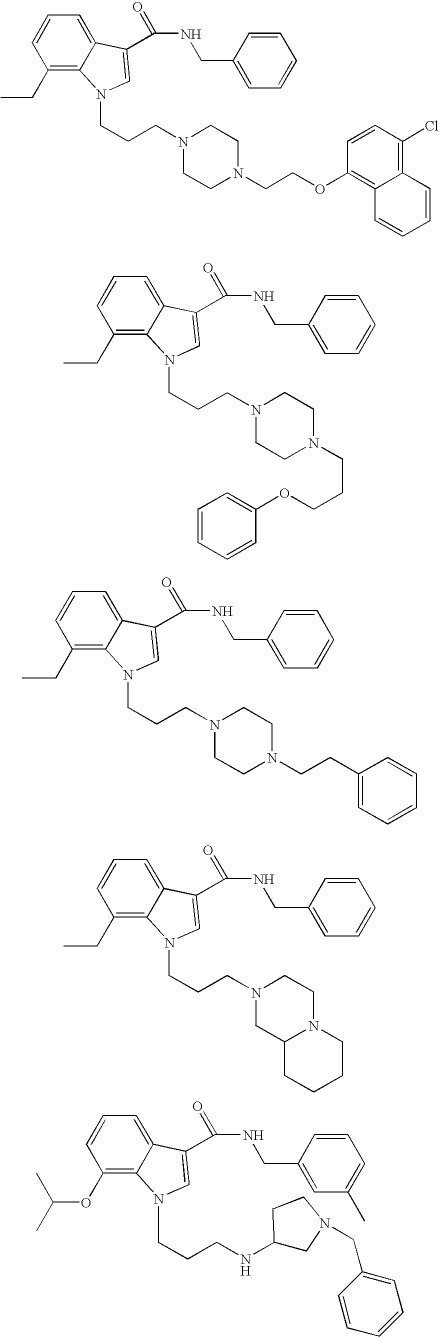

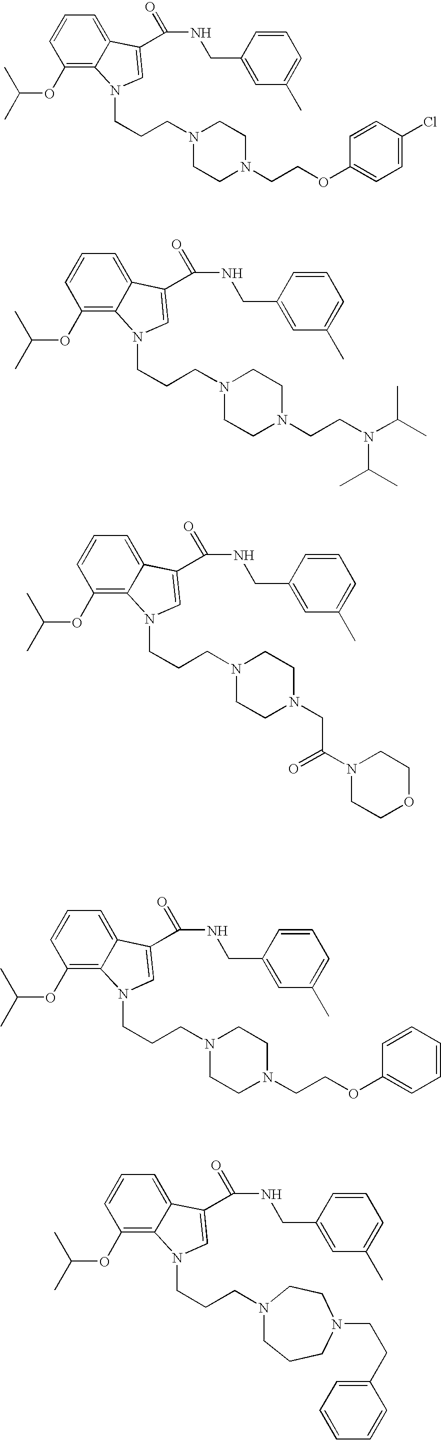

79. The compound of claim 1, wherein the compound is selected from the group consisting of:

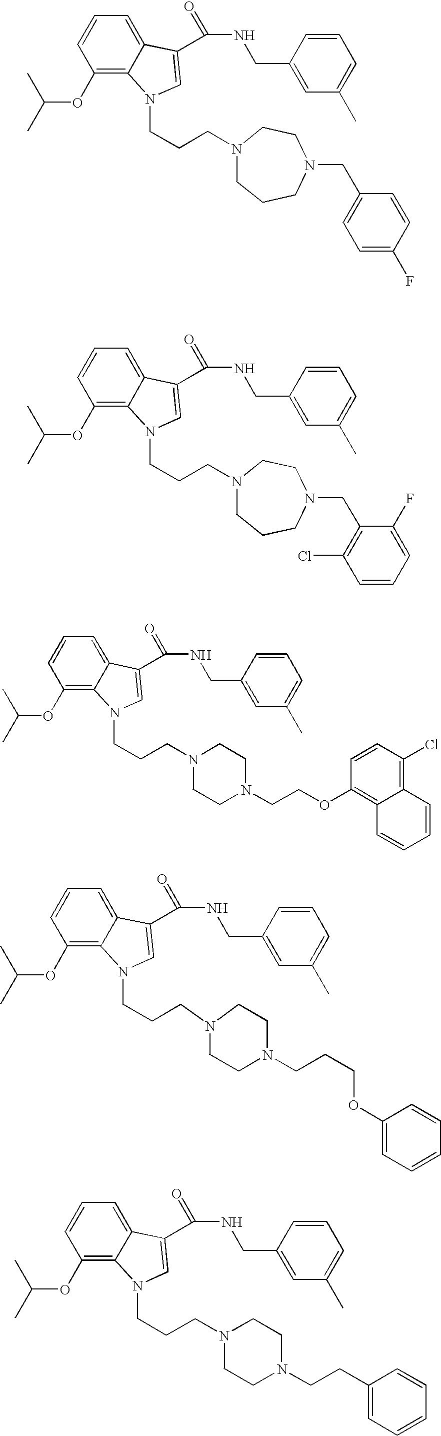

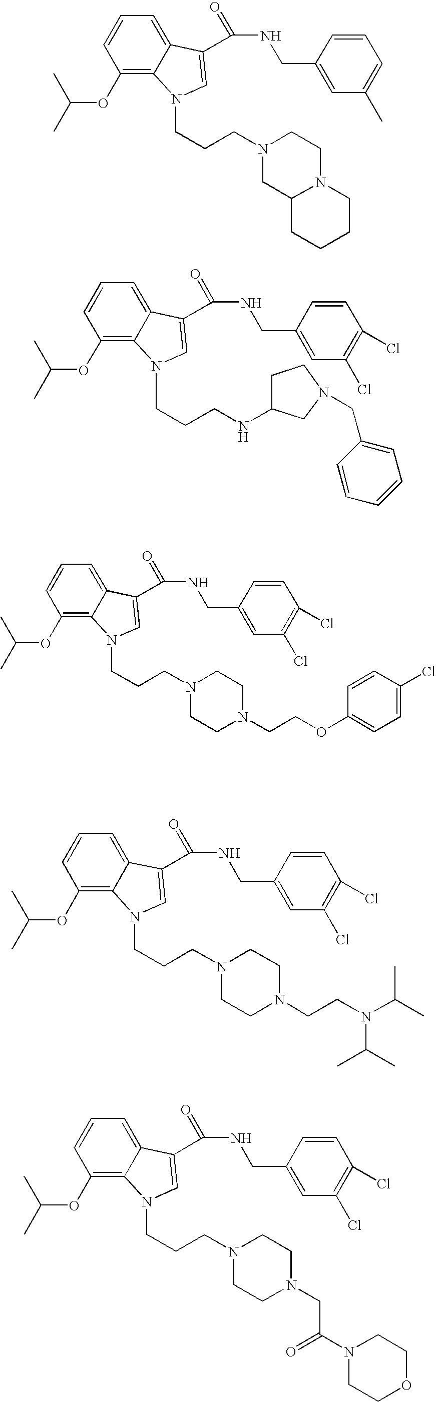

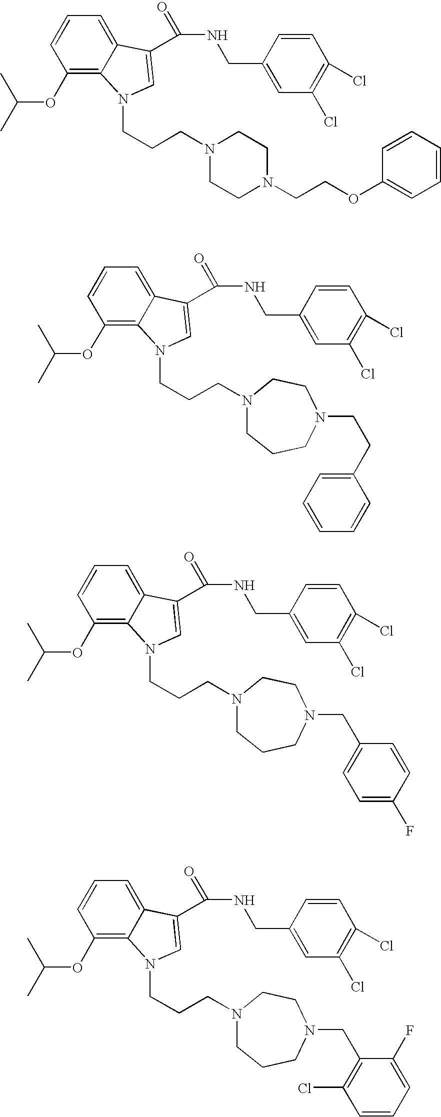

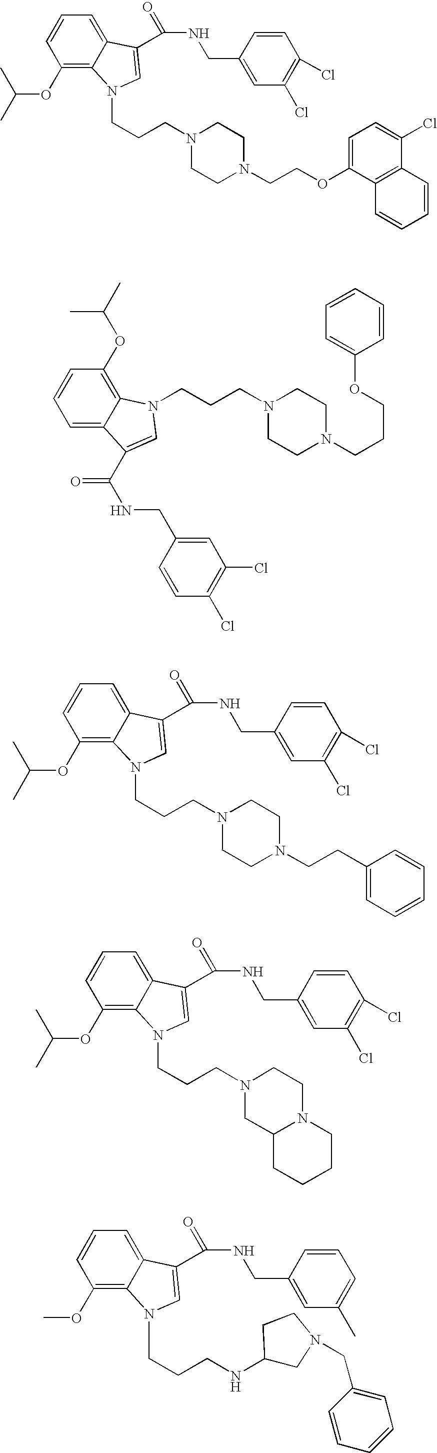

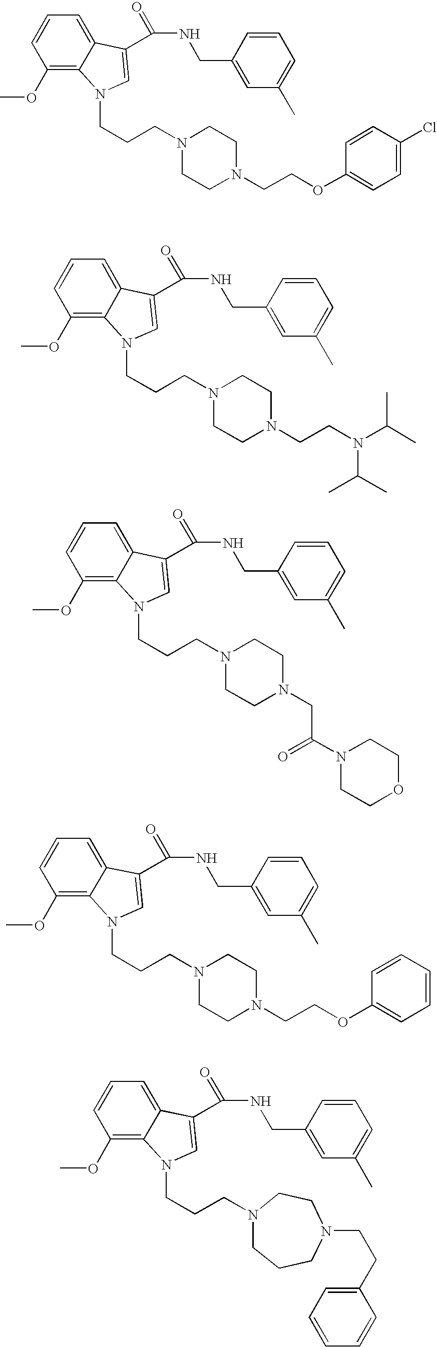

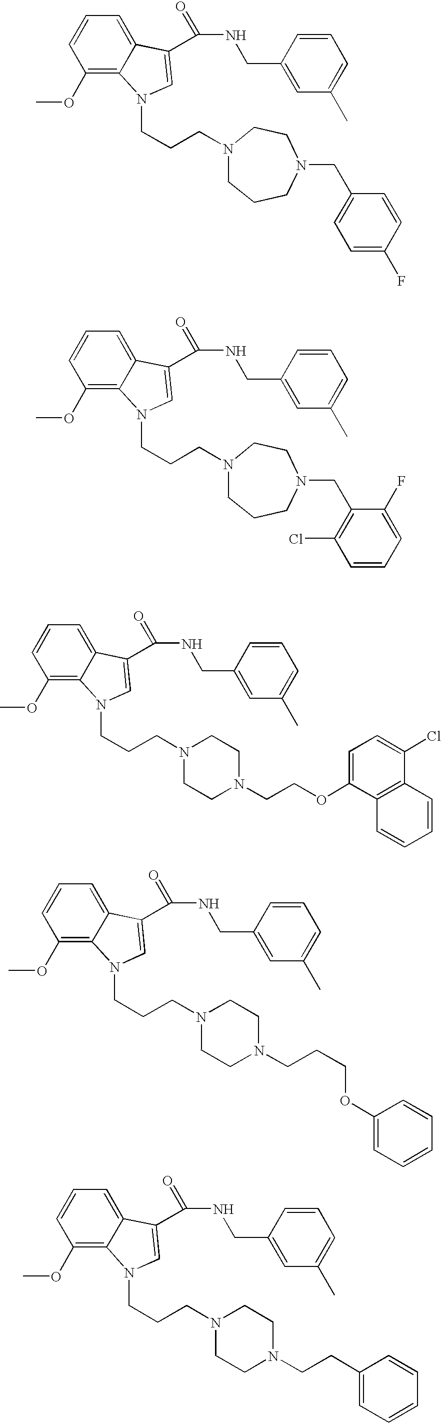

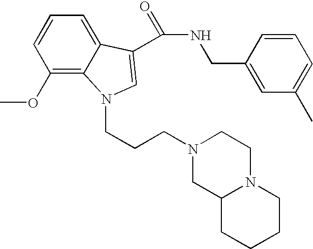

80. The compound of claim 1, wherein the compound is selected from the group consisting of:

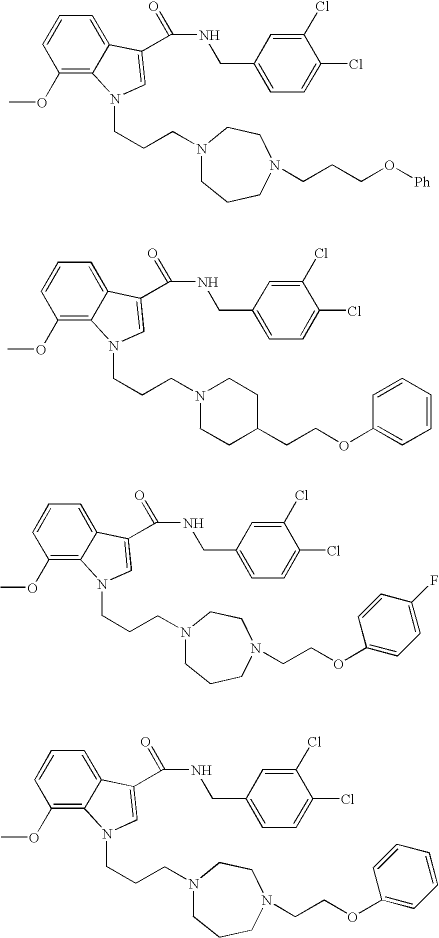

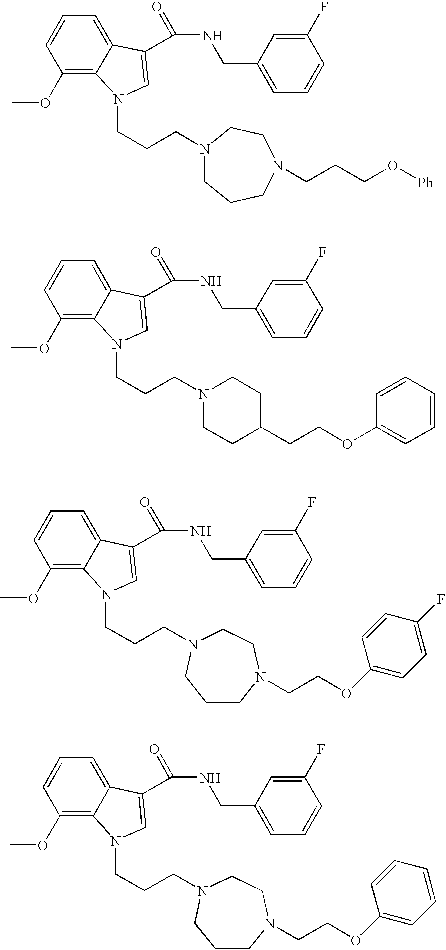

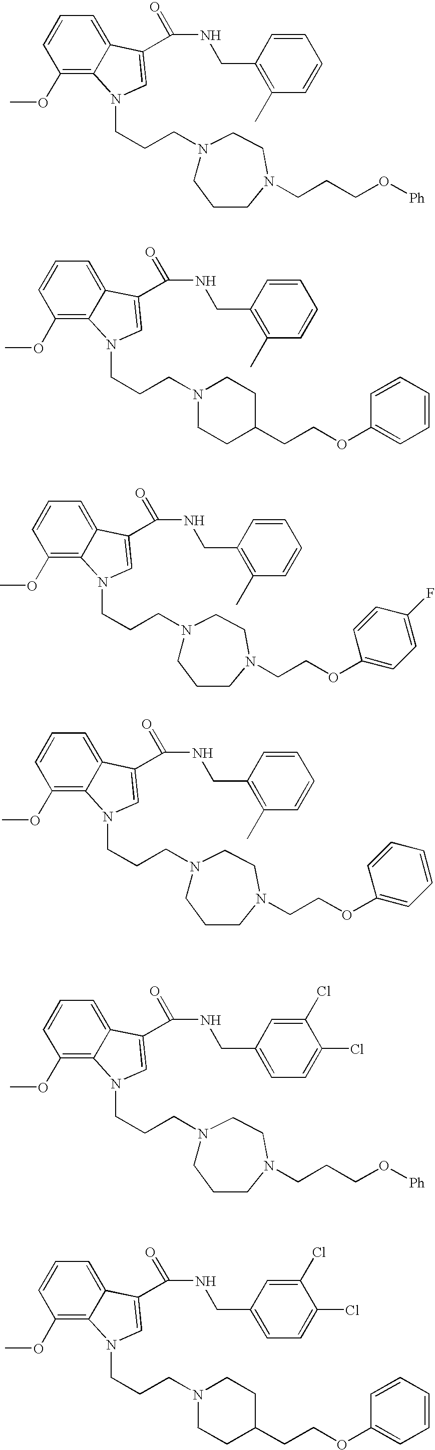

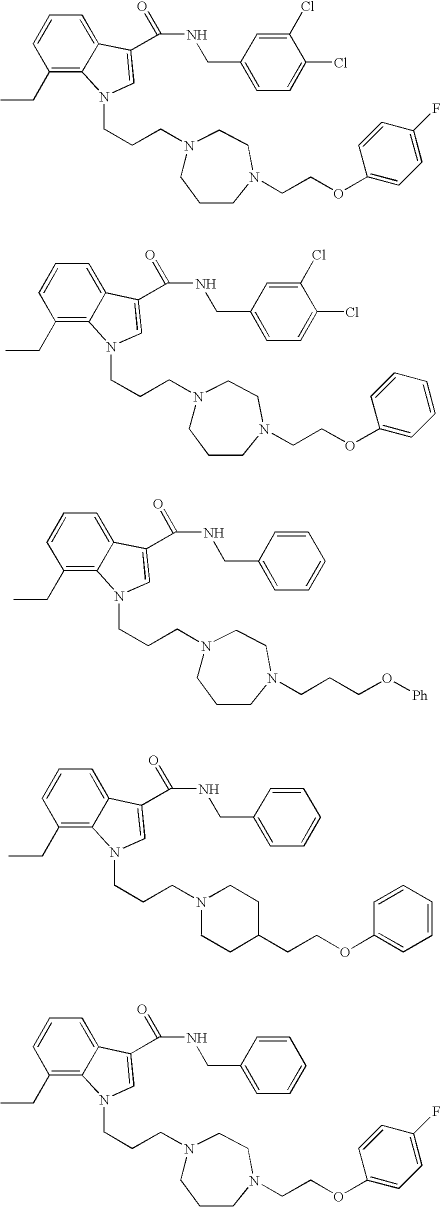

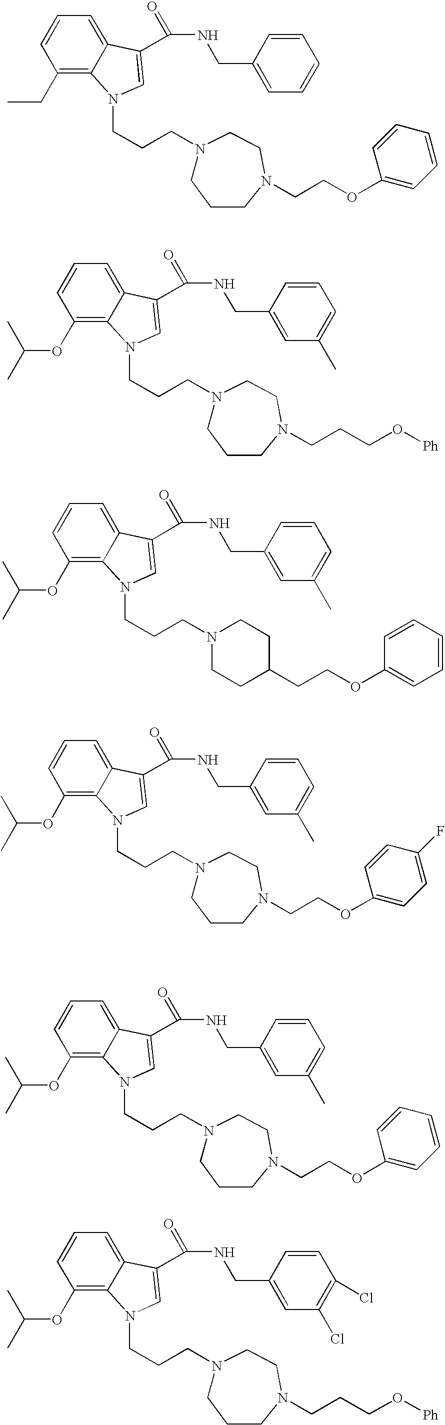

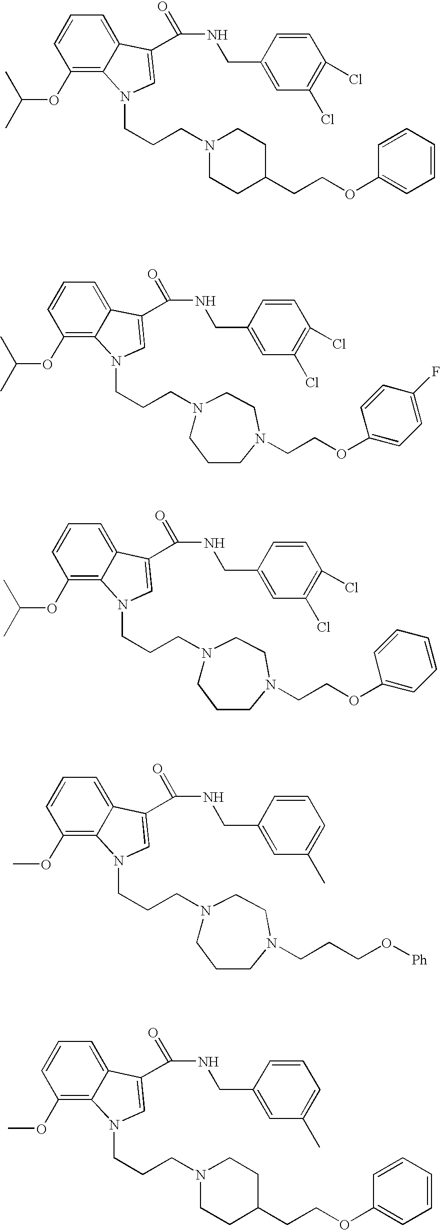

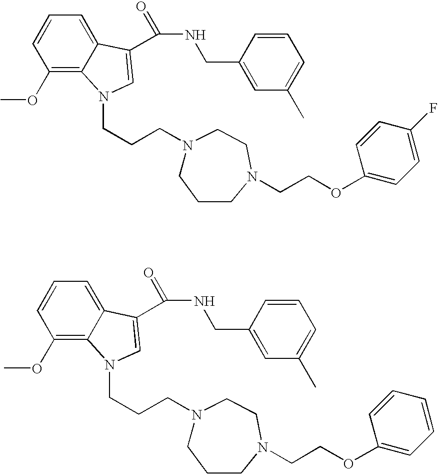

81. The compound of claim 1, wherein the compound is selected from the group consisting of:

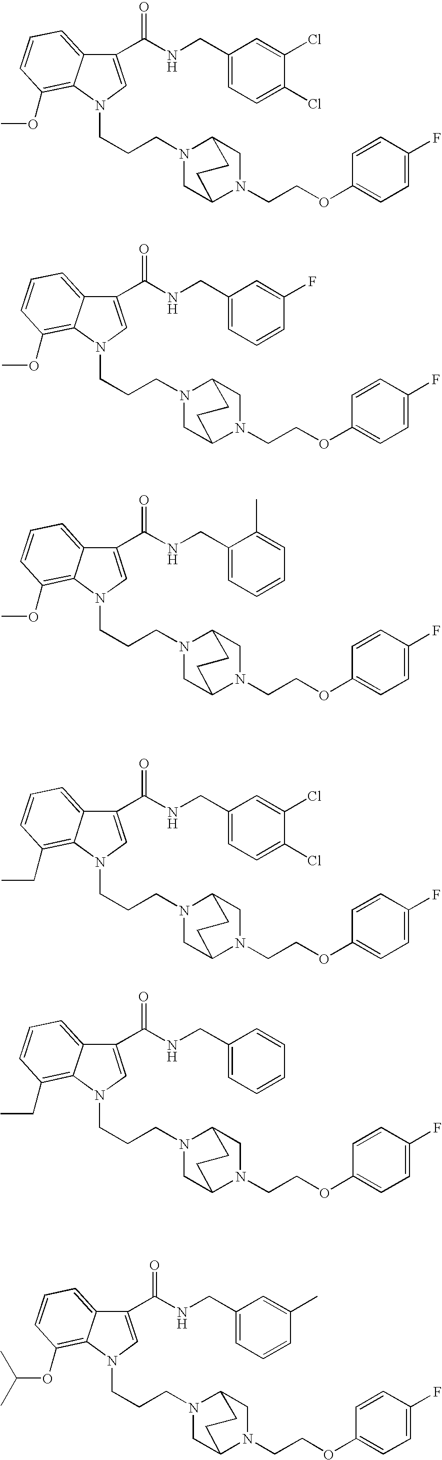

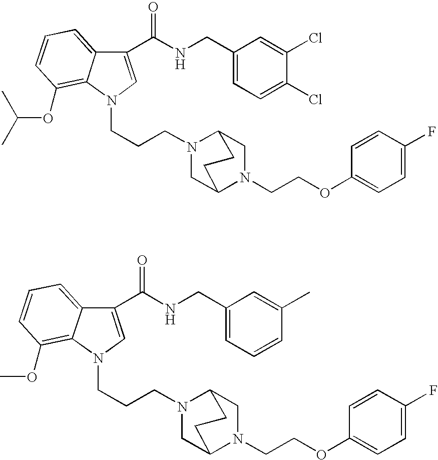

82. The compound of claim 1, wherein the compound is selected from the group consisting of:

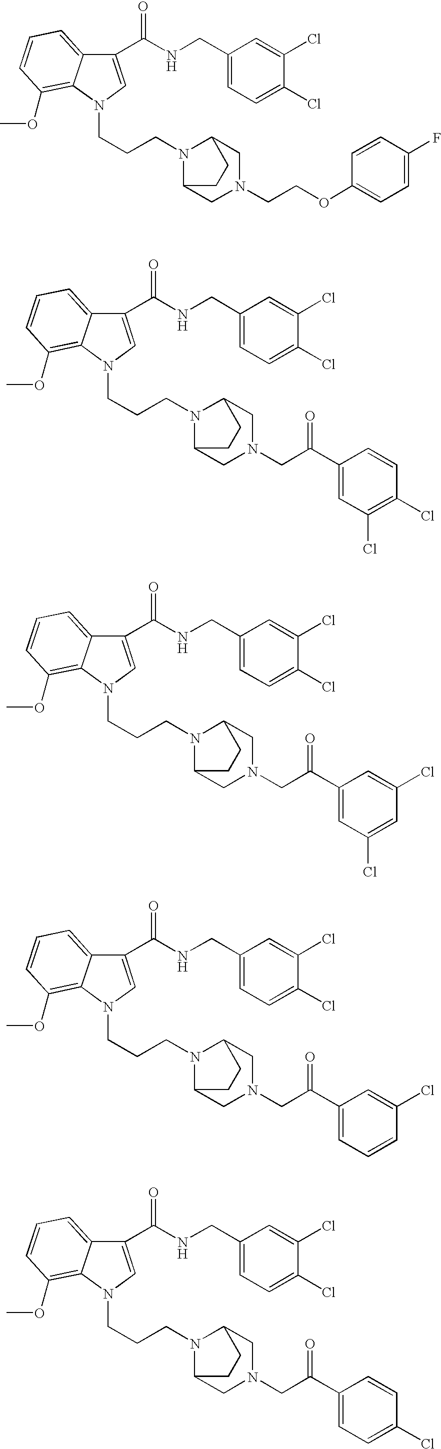

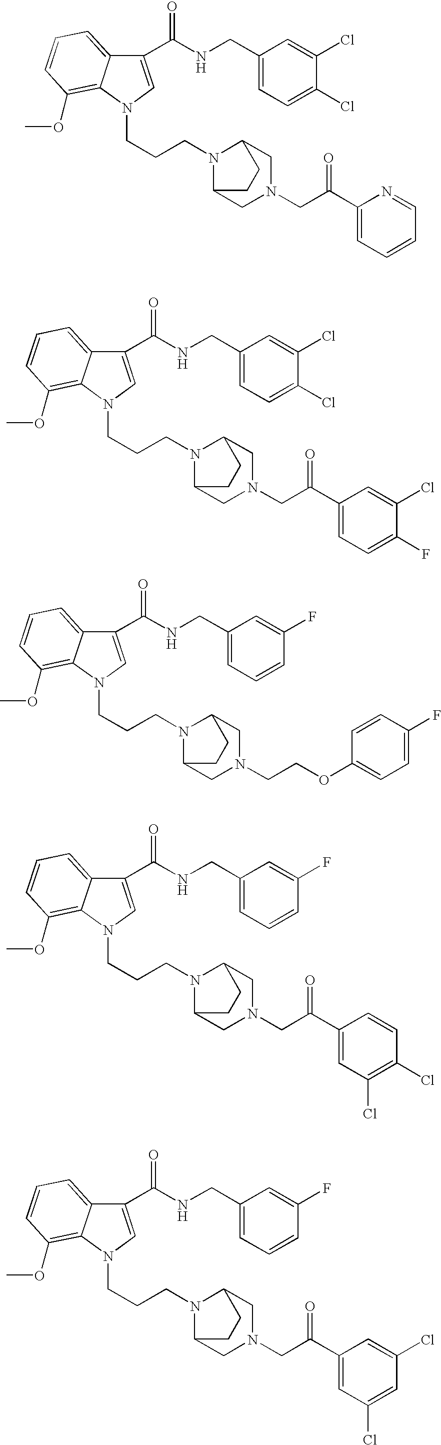

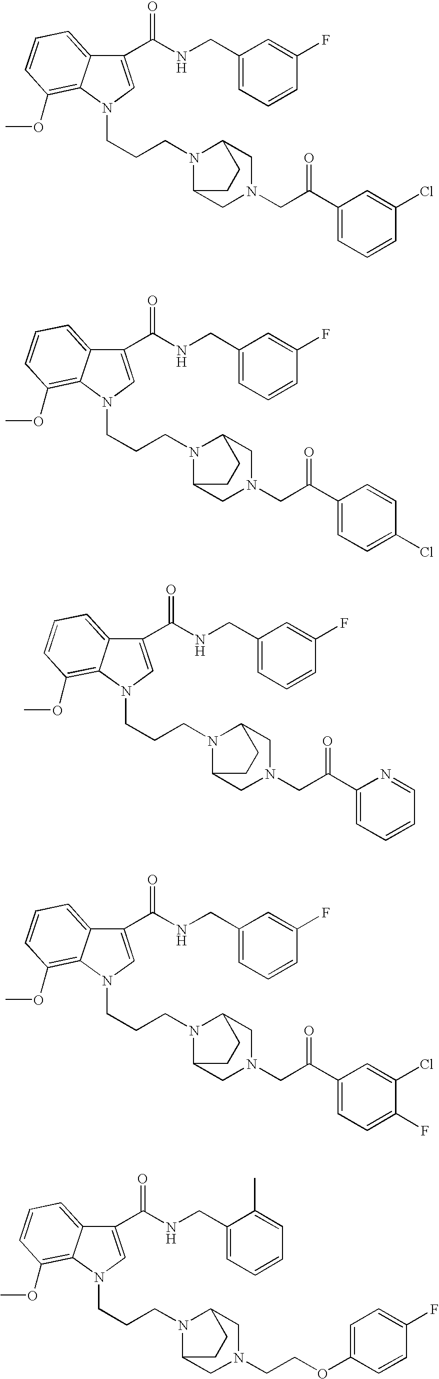

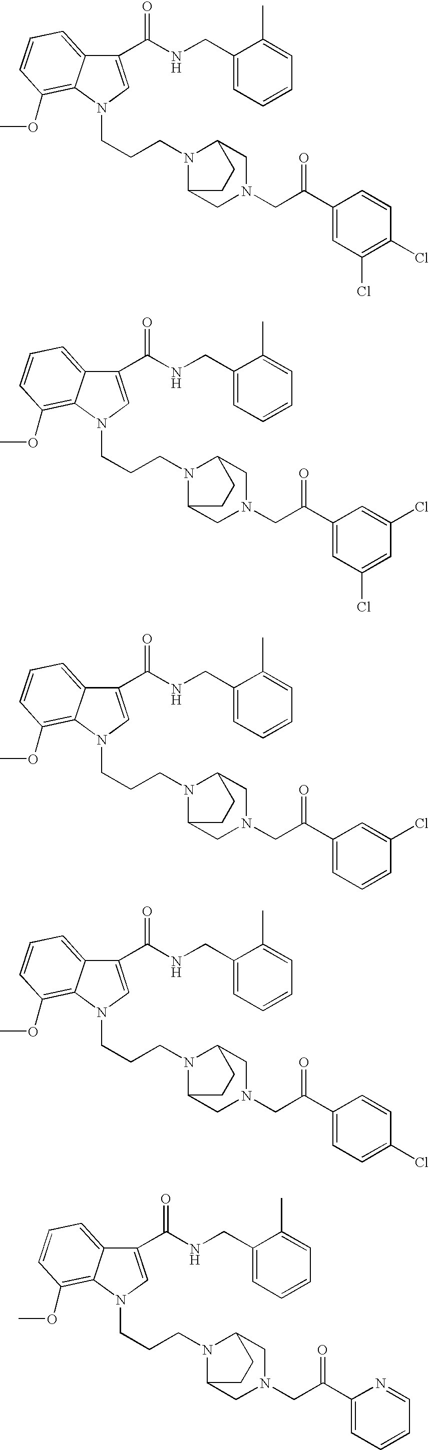

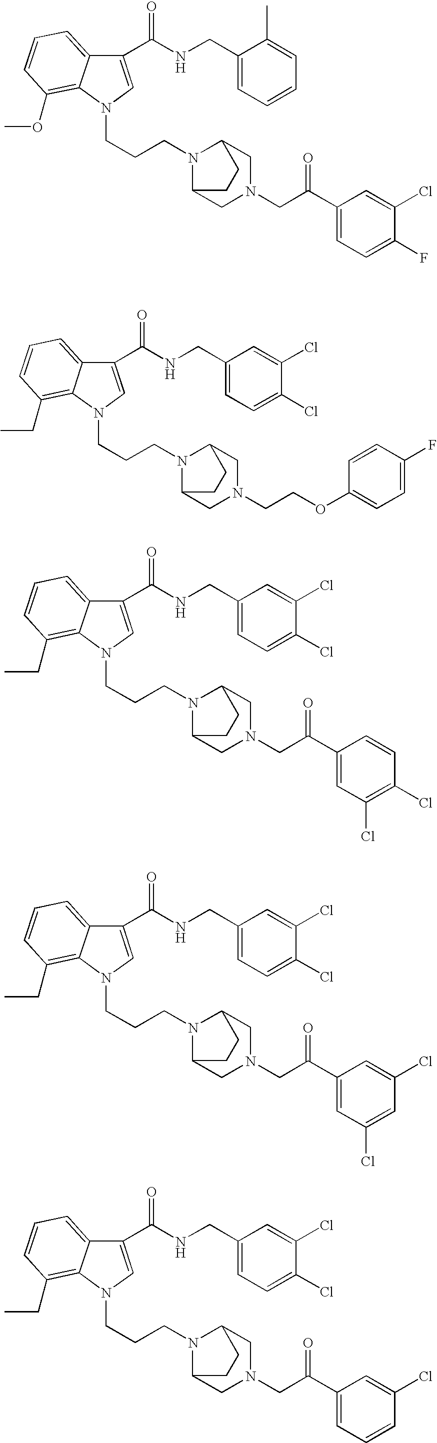

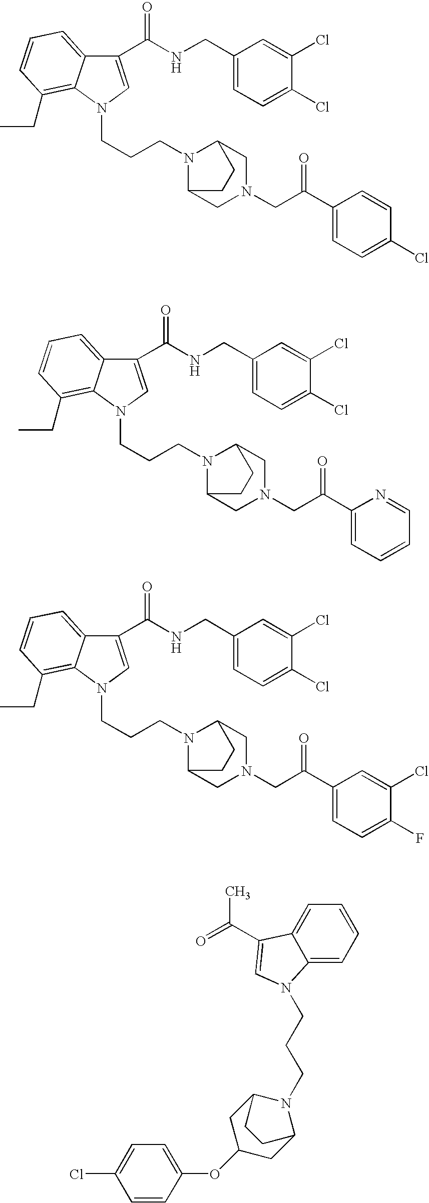

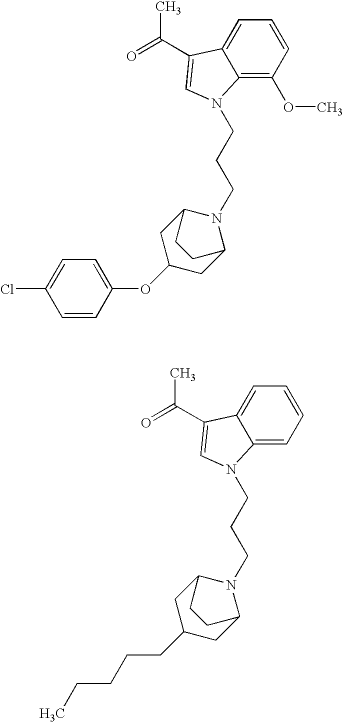

83. The compounds of claim 82, wherein the compounds have the following stereochemistry:

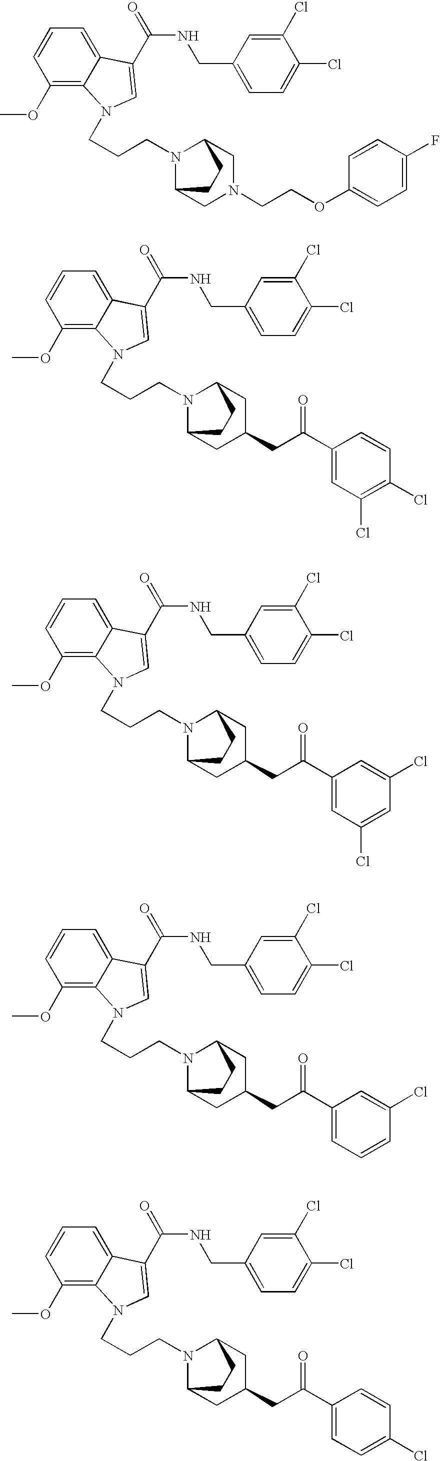

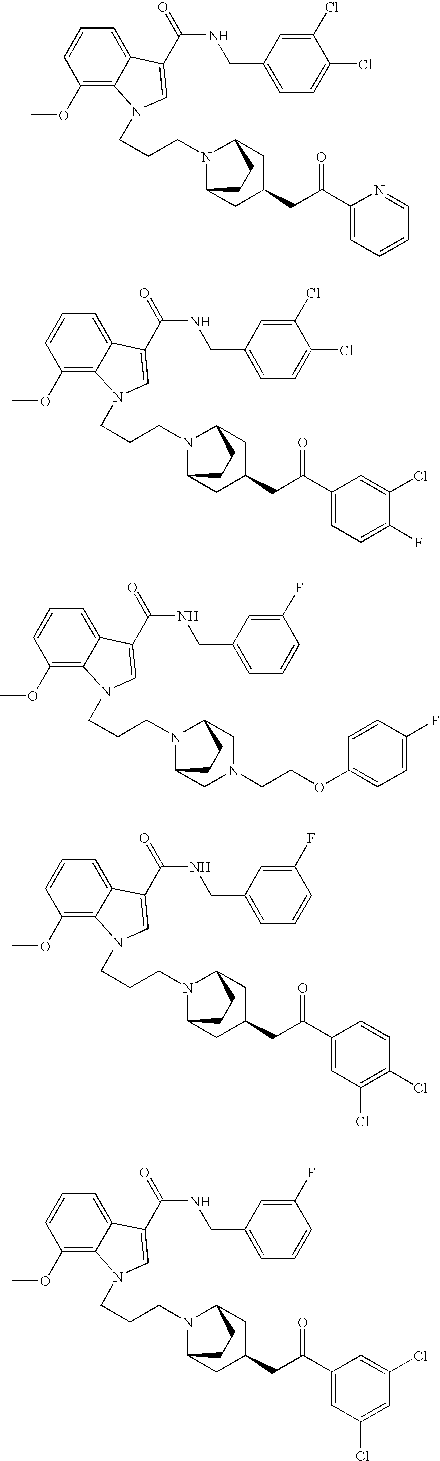

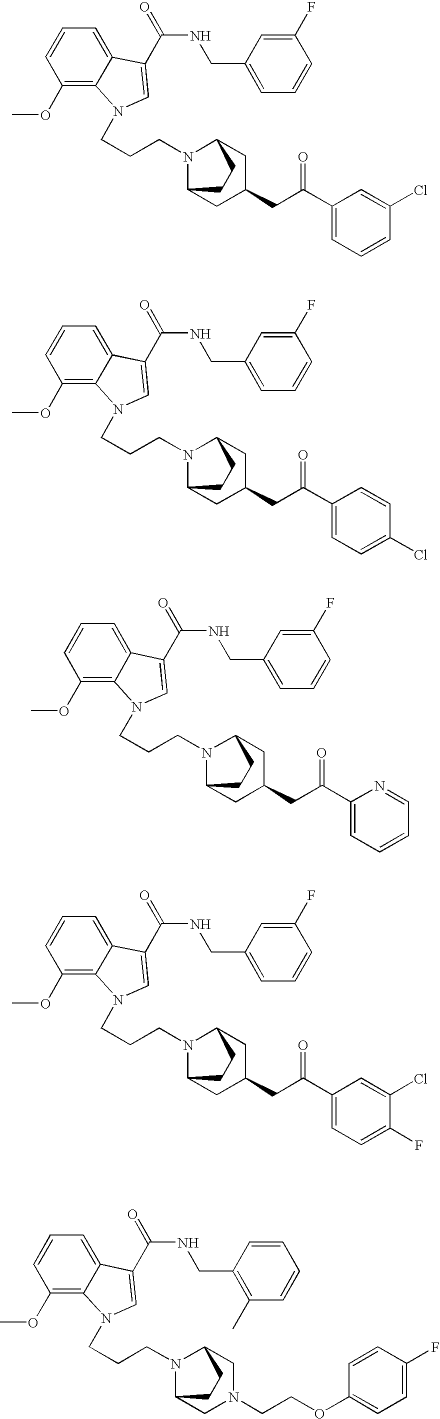

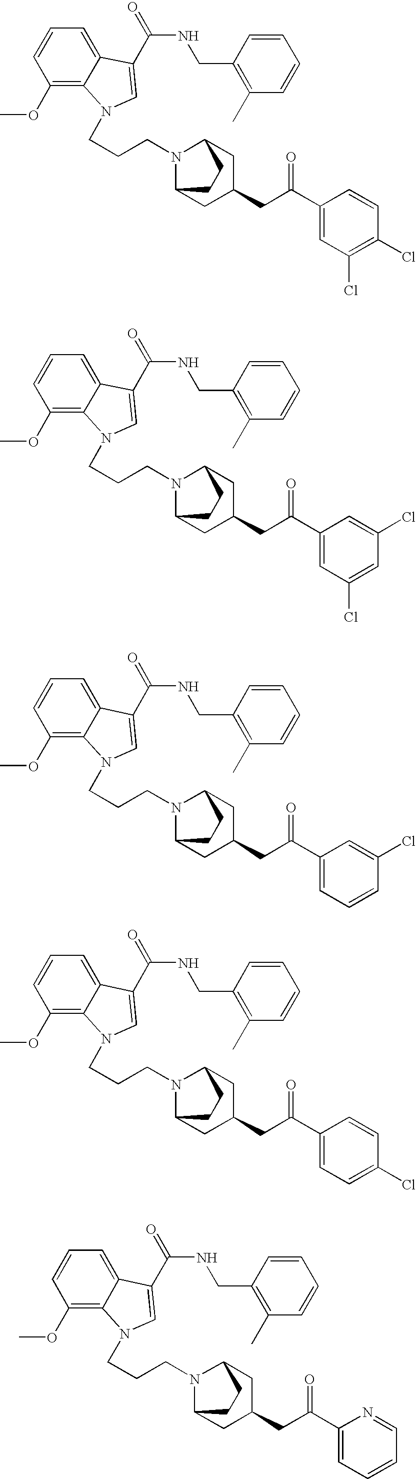

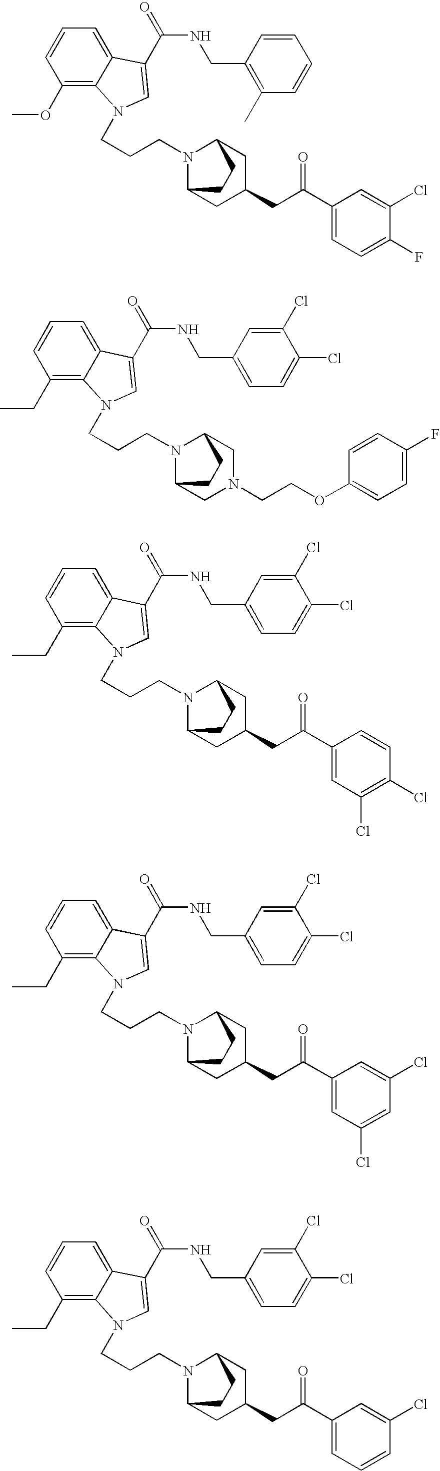

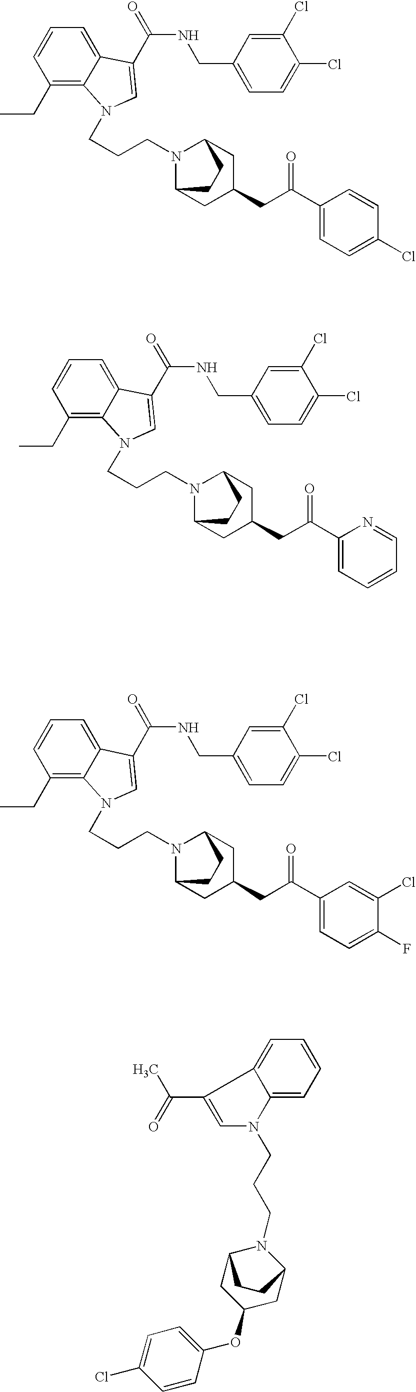

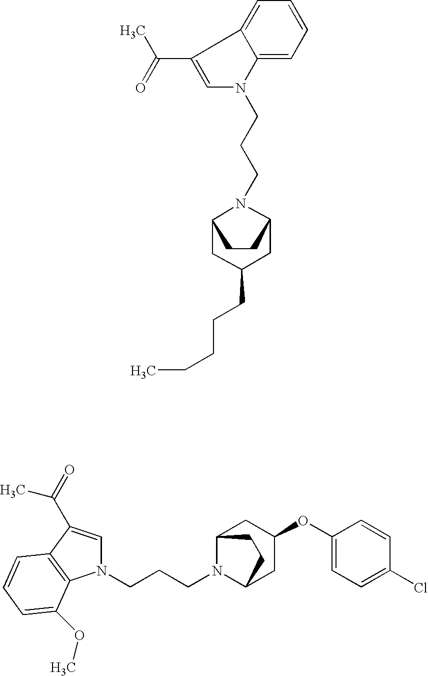

84. The compound of claim 1, wherein the compound is selected from the group consisting of:

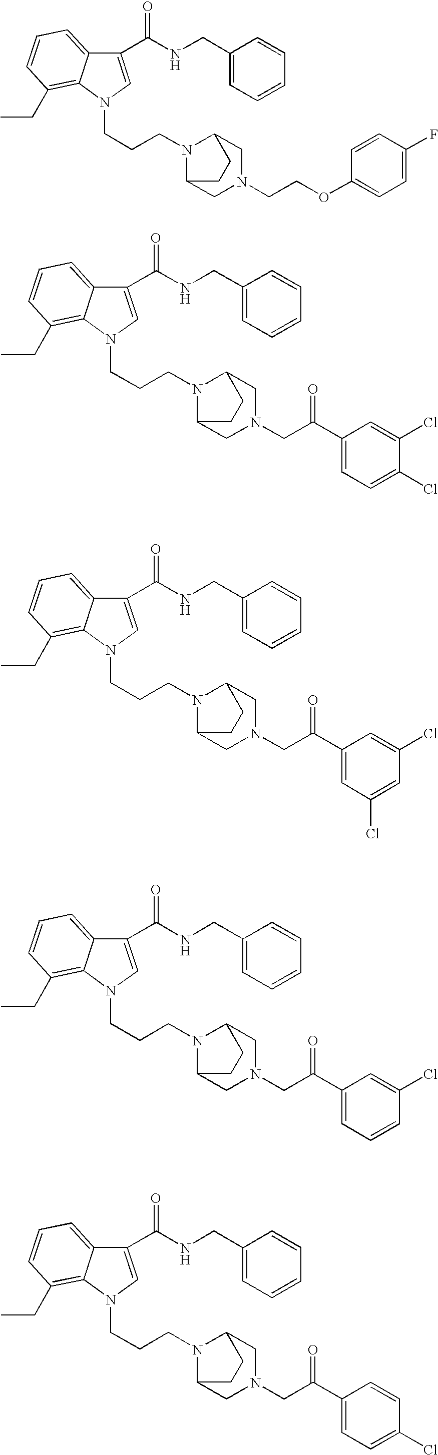

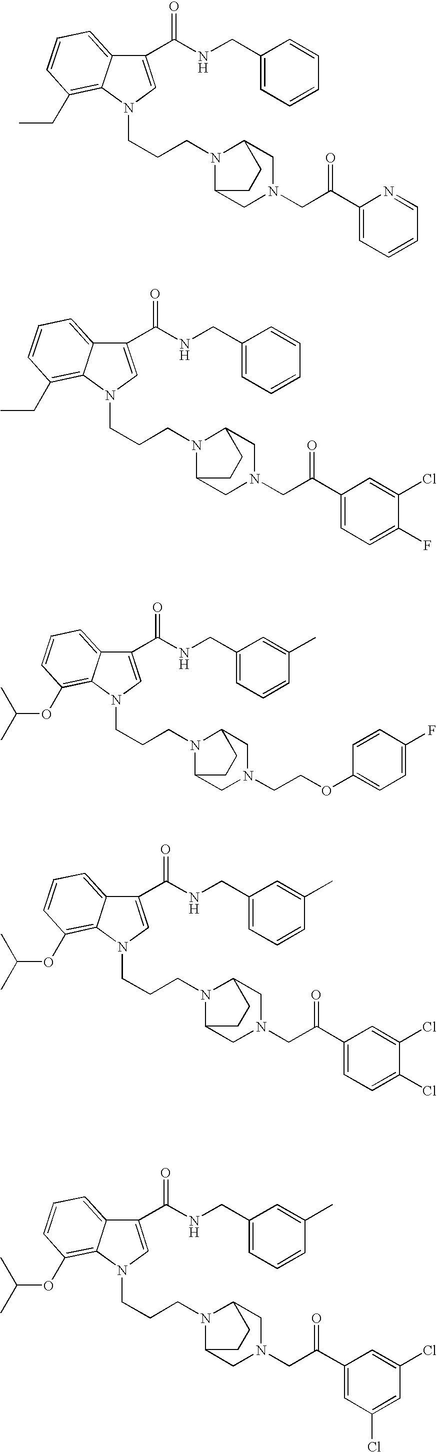

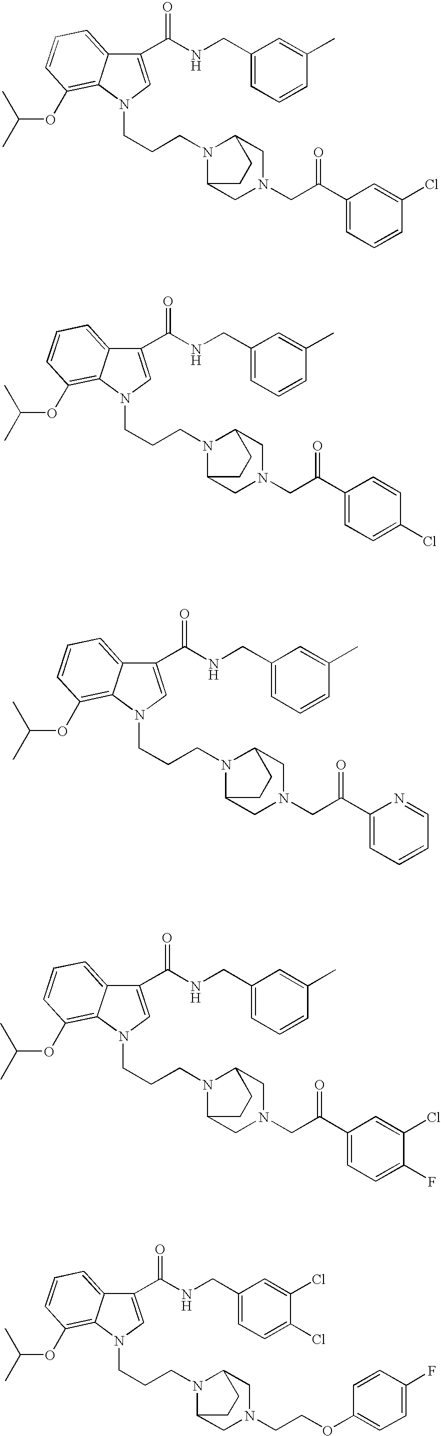

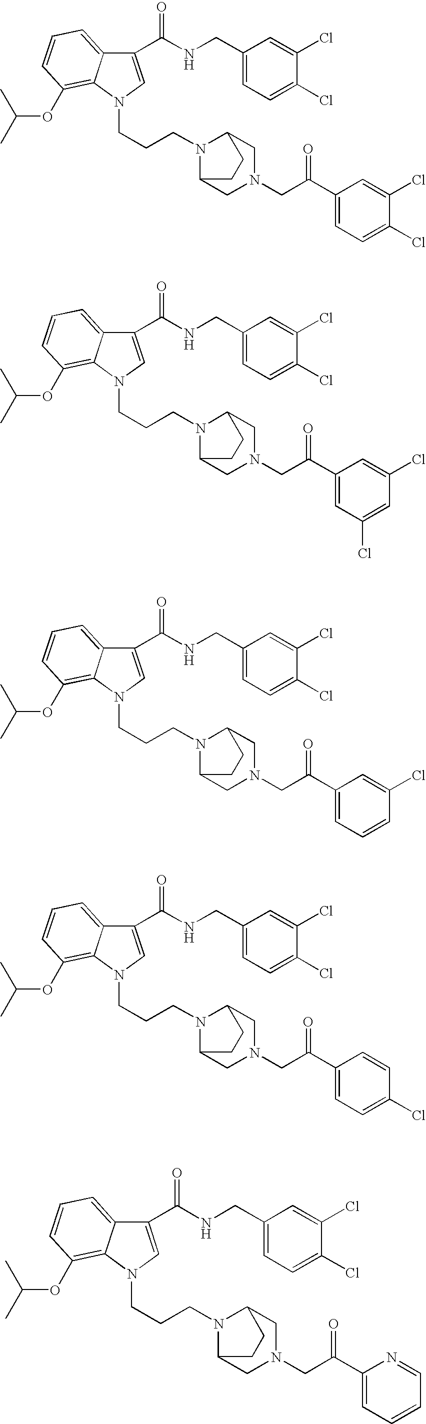

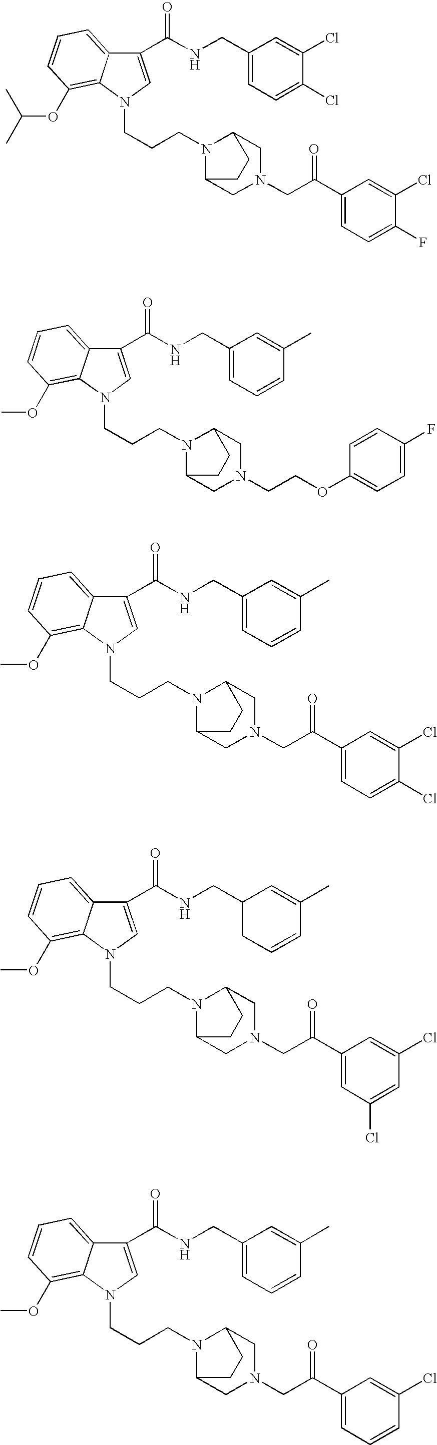

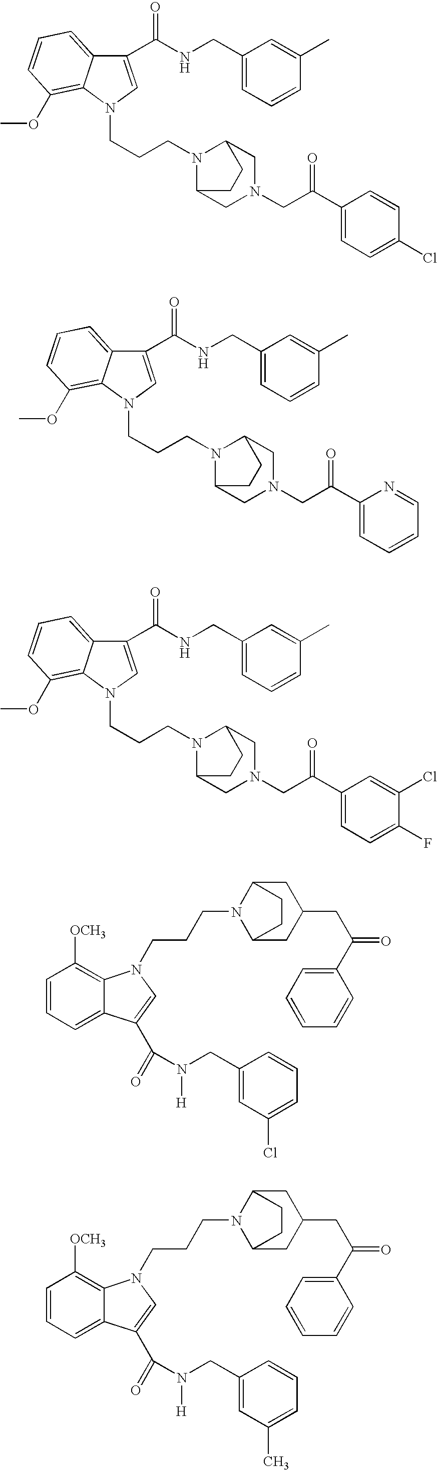

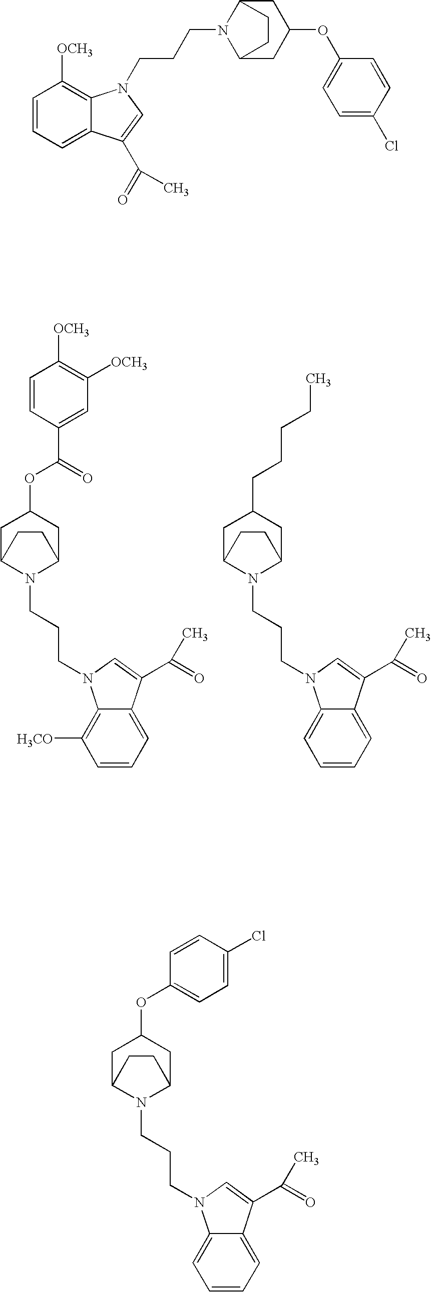

85. The compounds of claim 84, wherein the compounds have the following stereochemistry:

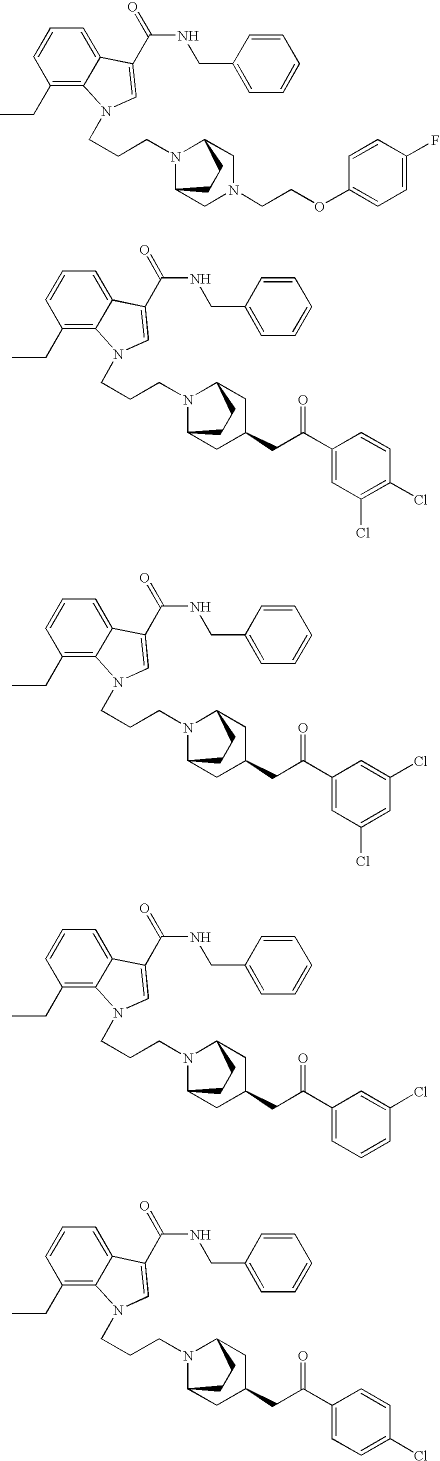

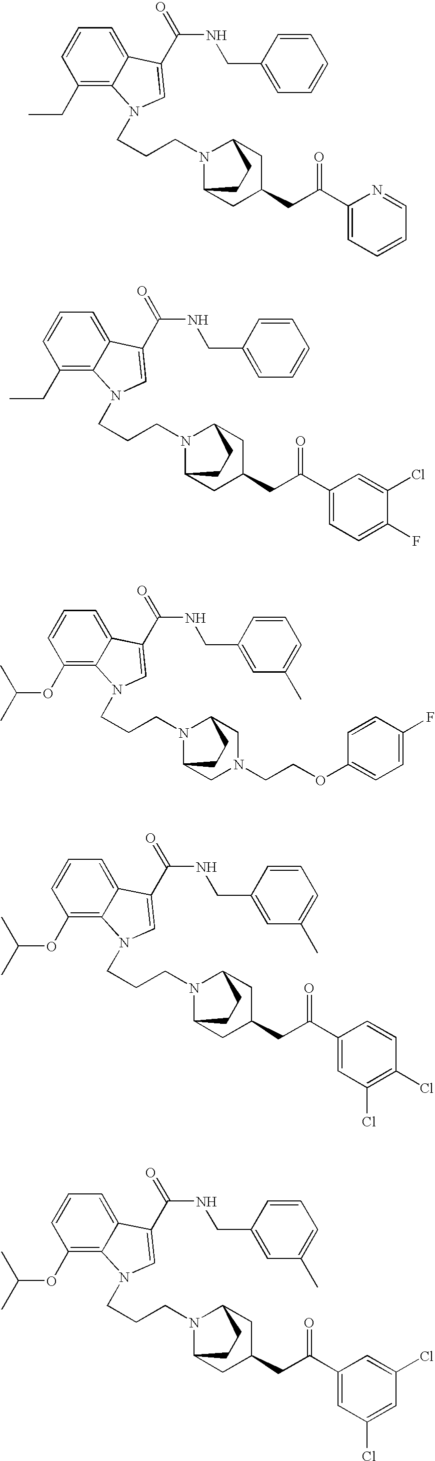

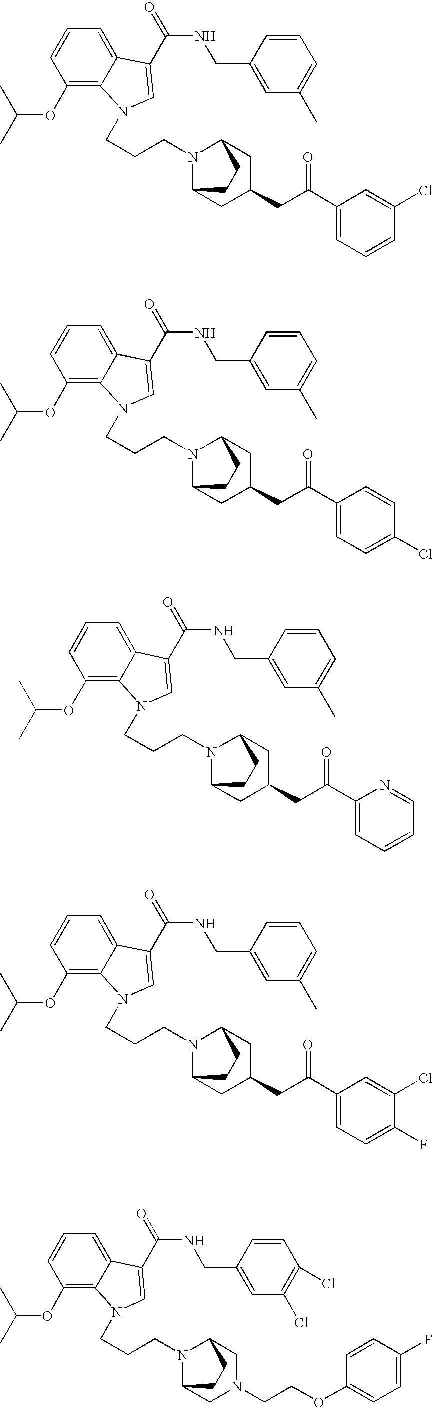

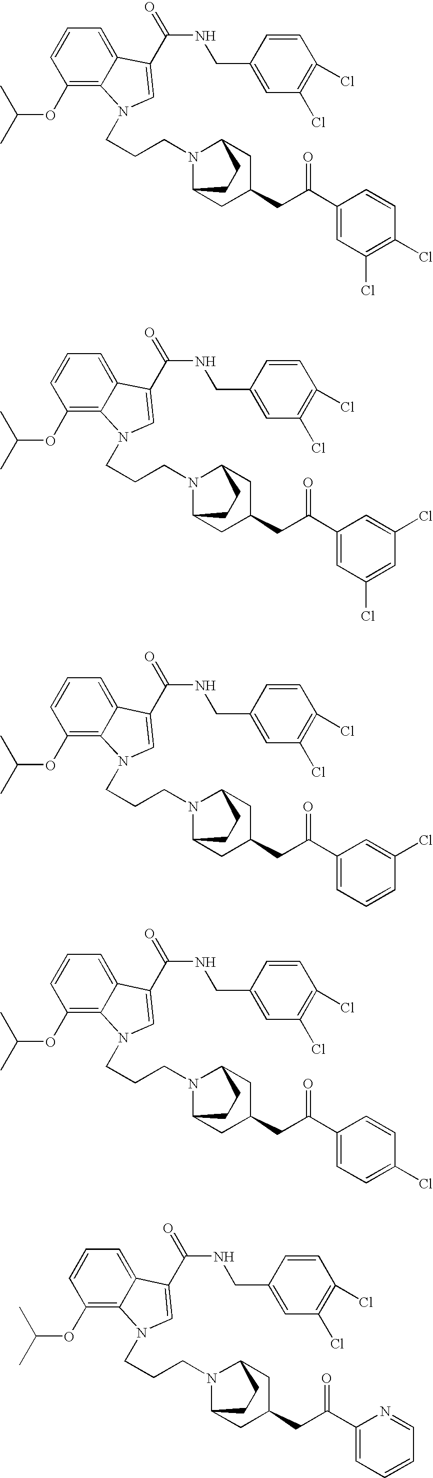

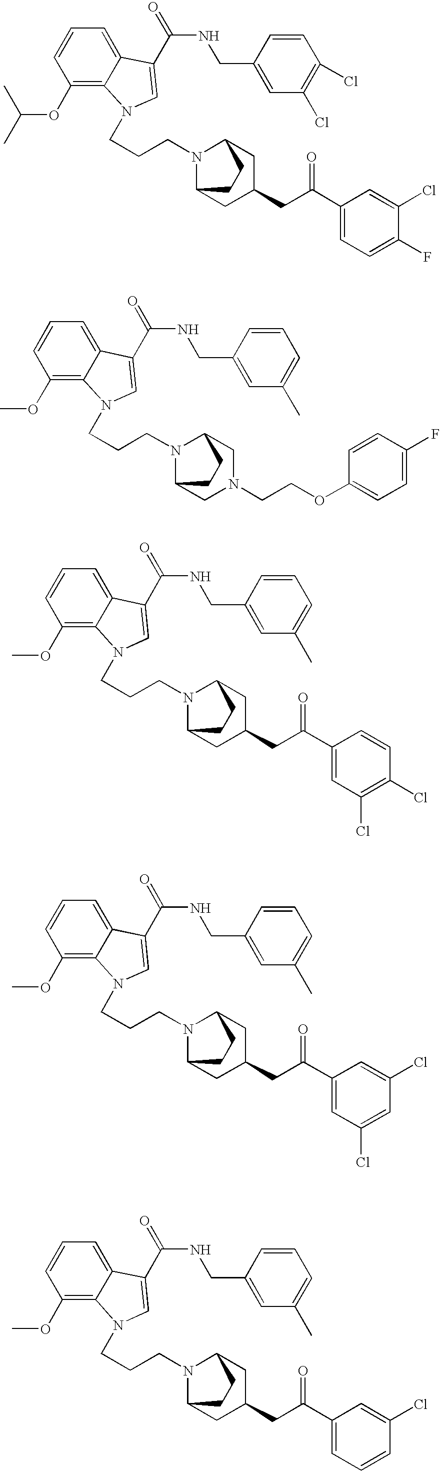

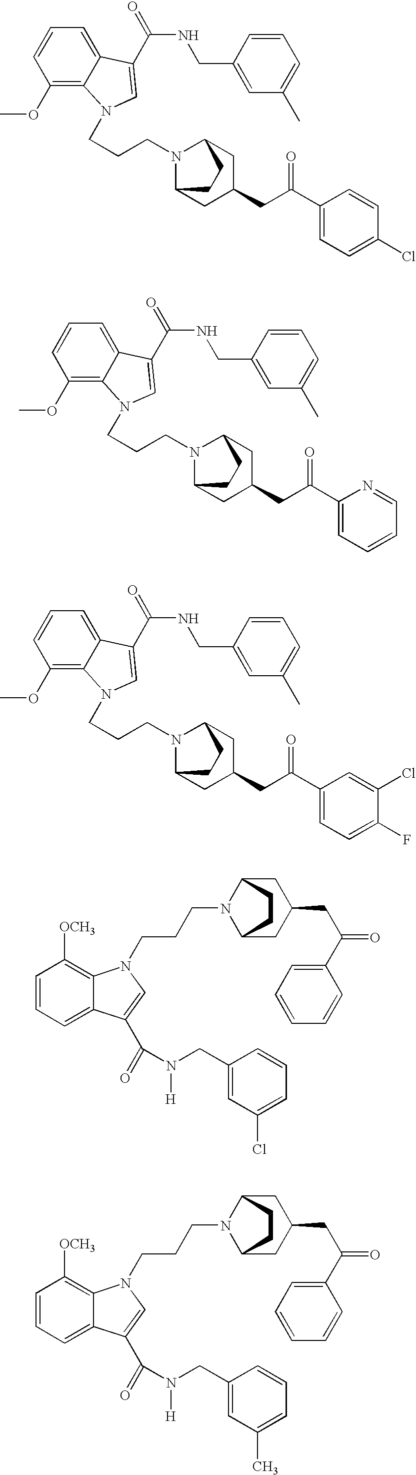

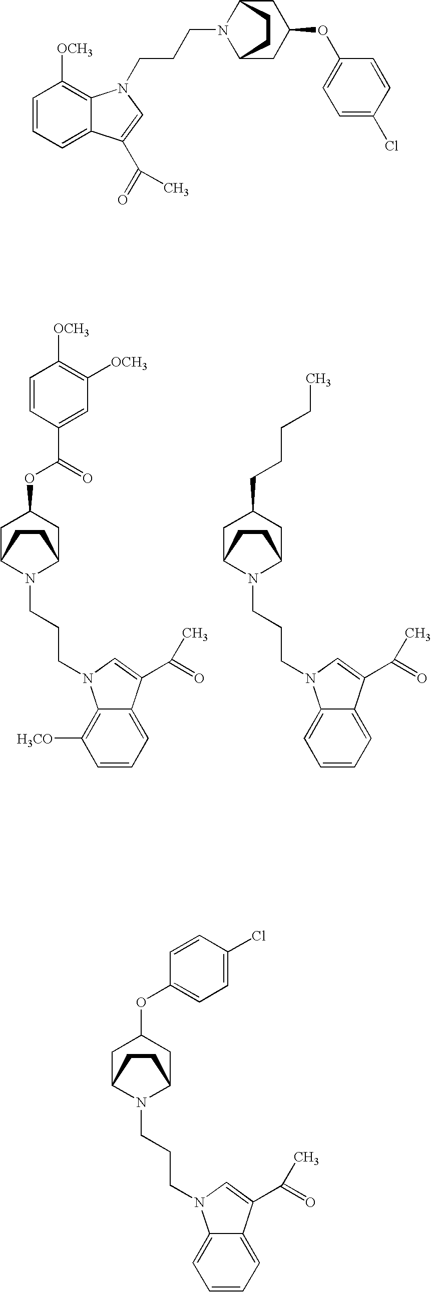

86. The compound of claim 1, wherein the compound is selected from the group consisting of:

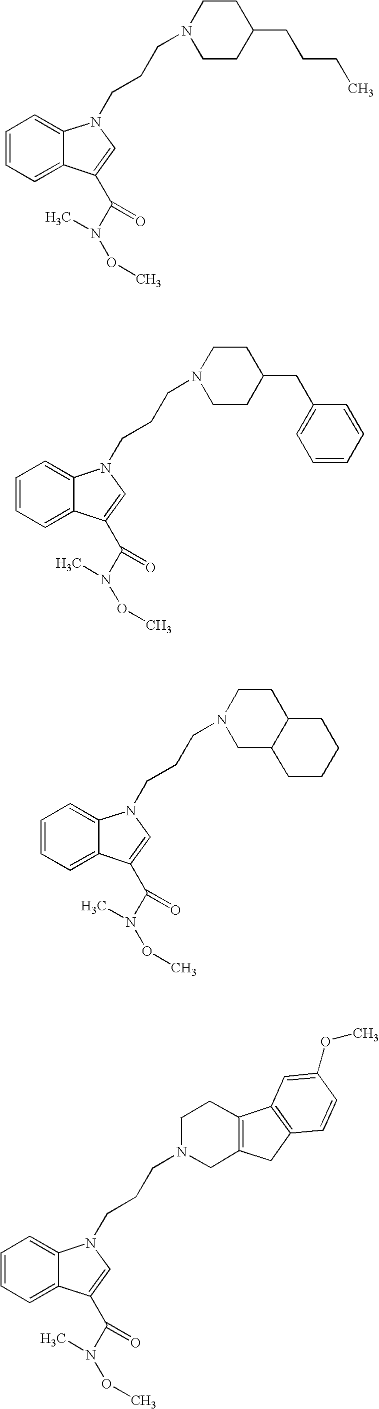

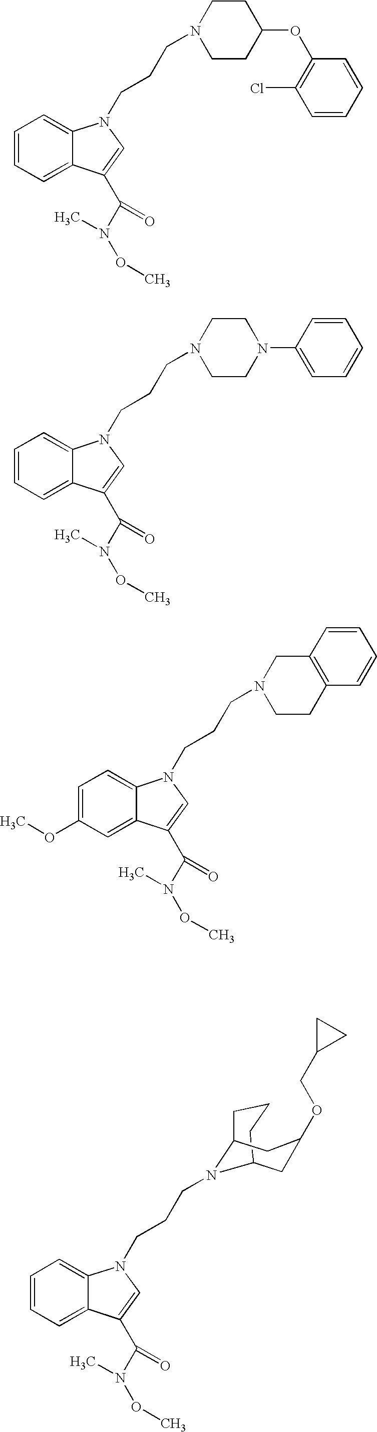

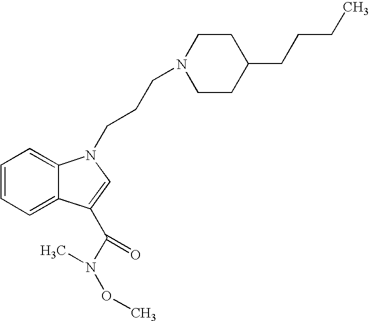

87. The compound of claim 1, wherein the compound is selected from the group consisting of a compound identified as C001, C002, C003, C004, C005, C006, C007, C008, C009, C010, C011, C012, C013, C014, C015, C016, C017, C018, C019, C020, C021, C022, C023, C024, C025, C026, C027, C028, C029, C030, C031, C032, C033, C034, C035, C036, C037, C038, C039, C040, C041, C042, C043, C044, C045, C046, C047, C048, C049, and C050.

88. The compound of claim 1, wherein the compound is selected from the group consisting of a compound identified as C051, C052, C053, C054, C055, C056, C057, C058, C059, C060, C061, C062, C063, C064, C065, C066, C067, C068, C069, C070, C071, C072, C073, C074, C075, C076, C077, C078, C079, C080, C081, C082, C083, C084, C085, C086, C087, C088, C089, C090, C091, C092, C093, C094, C095, C096, C097, C098, C099, and C100.

89. The compound of claim 1, wherein the compound is selected from the group consisting of a compound identified as C101, C102, C103, C104, C105, C106, C107, C108, C109, C010, C111, C112, C113, C114, C115, C116, C117, C118, C119, C120, C121, C122, C123, C124, C125, C126, C127, C128, C129, C130, C131, C132, C133, C134, C135, C136, C137, C138, C139, C140, C141, C142, C143, C144, C145, C146, C147, C148, C149, and C150.

90. The compound of claim 1, wherein the compound is selected from the group consisting of a compound identified as C151, C152, C153, C154, C155, C156, C157, C158, C159, C160, C161, C162, C163, C164, C165, C166, C167, C168, C169, C170, C171, C172, C173, C174, C175, C176, C177, C178, C179, C180, C181, C182, C183, C184, C185, C186, C187, C188, C189, C190, C191, C192, C193, C194, C195, C196, C197, C198, C199, and C200.

91. The compound of claim 1, wherein the compound is selected from the group consisting of a compound identified as C201, C202, C203, C204, C205, C206, C207, C208, C209, C210, C211, C212, C213, C214, C215, C216, C217, C218, C219, C220, C221, C222, C223, C224, C225, C226, C227, C228, C229, C230, C231, C232, C233, C234, C235, C236, C237, C238, C239, C240, C241, C242, C243, C244, C245, C246, C247, C248, C249, and C250.

92. The compound of claim 1, wherein the compound is selected from the group consisting of a compound identified as C251, C252, C253, C254, C255, C256, C257, C258, C259, C260, C261, C262, C263, C264, C265, C266, C267, C268, C269, C270, C271, C272, C273, C274, C275, C276, C277, C278, C279, C280, C281, C282, C283, C284, C285, C286, C287, C288, C289, C290, C291, C292, C293, C294, C295, C296, C297, C298, C299, and C300.

93. The compound of claim 1, wherein the compound is selected from the group consisting of a compound identified as C301, C302, C303, C304, C305, C306, C307, C308, C309, C310, C311, C312, C313, C314, C315, C316, C317, C318, C319, C320, C321, C322, C323, C324, C325, C326, C327, C328, C329, C330, C331, C332, C333, C334, C335, C336, C337, C338, C339, C340, C341, C342, C343, C344, C345, C346, C347, C348, C349, and C350.

94. The compound of claim 1, wherein the compound is selected from the group consisting of a compound identified as C351, C352, C353, C354, C355, C356, C357, C358, C359, C360, C361, C362, C363, C364, C365, C366, C367, C368, C369, C370, C371, C372, C373, C374, C375, C376, C377, C378, C379, C380, C381, C382, C383, C384, C385, C386, C387, C388, C389, C390, C391, C392, C393, C394, C395, C396, C397, C398, C399, and C400.

95. The compound of claim 1, wherein the compound is selected from the group consisting of a compound identified as C401, C402, C403, C404, C405, C406, C407, C408, C409, C410, C411, C412, C413, C414, C415, C416, C417, C418, C419, C420, C421, C422, C423, C424, C425, C426, C427, C428, C429, C430, C431, C432, C433, C434, C435, C436, C437, C438, C439, C440, C441, C442, C443, C444, C445, C446, C447, C448, C449, and C450.

96. The compound of claim 1, wherein the compound is selected from the group consisting of a compound identified as C451, C452, C453, C454, C455, C456, C457, C458, C459, C460, C461, C462, C463, C464, C465, C466, C467, C468, C469, C470, C471, C472, C473, C474, C475, C476, C477, C478, C479, C480, C481, C482, C483, C484, C485, C486, C487, C488, C489, C490, C491, C492, C493, C494, C495, C496, C497, C498, C499, and C500.

97. The compound of claim 1 wherein the compound is selected from the group consisting of a compound identified as C501, C502, C503, C504, C505, C506, C507, C508, C509, C510, C511, C512, C513, C514, C515, C516, C517, C518, C519, C520, C521, C522, C523, C524, C525, C526, C527, C528, C529, C530, C531, C532, C533, C534, C535, C536, C537, C538, C539, C540, C541, C542, C543, C544, C545, C546, C547, C548, C549, and C550.

98. The compound of claim 1, wherein the compound is selected from the group consisting of a compound identified as C551, C552, C553, C554, C555, C556, C557, C558, C559, C560, C561, C562, C563, C564, C565, C566, C567, C568, C569, C570, C571, C573, C574, C575, C576, C577, C57$, C579, C580, C581, C582, C583, C584, C585, C586, C587, C588, C589, C590, C591, C592, C593, C594, C595, C596, C597, C598, C599, and C600.

99. The compound of claim 1, wherein the compound is selected from the group consisting of a compound identified as C601, C602, C603, C604, C605, C606, C607, C608, C609, C610, C611, C612, C613, C614, C615, C616, C617, C618, C619, C620, C621, C622, C623, C624, C625, C626, C627, C628, C629, C630, C631, C632, C633, C634, C635, C636, C637, C638, C639, C640, C641, C642, C643, C644, C645, C646, C647, C648, C649, and C650.

100. The compound of claim 1, wherein the compound is selected from the group consisting of a compound identified as C651, C652, C653, C654, C655, C656, C657, C658, C659, C660, C661, C662, C663, C664, C665, C666, C667, C668, C669, C670, C671, C672, C673, C674, C675, C676, C677, C678, C679, C680, C681, C682, C683, C684, C685, C686, C687, C688, C689, C690, C691, C692, C693, C694, C695, C696, C697, C698, C699, and C700.

101. The compound of claim 1, wherein the compound is selected from the group consisting of a compound identified as C701, C702, C703, C704, C705, C706, C707, C708, C709, C710, C711, C712, C713, C714, C715, C716, C717, C718, C719, C720, C721, C722, C723, C724, C725, C726, C727, C728, C729, C730, C731, C732, C733, C734, C735, C736, C737, C738, C739, C740, C741, C742, C743, C744, C745, C746, C747, C748, C749, and C750.

102. The compound of claim 1, wherein the compound is selected from the group consisting of a compound identified as C751, C752, C753, C754, C755, C756, C757, C758, C759, C760, C761, C762, C763, C764, C765, C766, C767, C768, C769, C770, C771, C772, C773, C774, C775, C776, C777, C778, C779, C780, C781, C782, C783, C784, C785, C786, C787, C788, C789, C790, C791, C792, C793, C794, C795, C796, C797, C798, C799, and C800.

103. The compound of claim 1, wherein the compound is selected from the group consisting of a compound identified as C801, C802, C803, C804, C805, C806, C807, C808, C809, C810, C811, C812, C813, C814, C815, C816, C817, C818, C819, C820, C821, C822, C823, C824, C825, C826, C827, C828, C829, C830, C831, C832, C833, C834, C835, C836, C837, C838, C839, C840, C841, C842, C843, C901, C902, C903, C904, and C905.

104. The compound of claim 1, wherein the compound is selected from the group consisting of a compound identified as C844, C845, C846, C847, C848, C849, C850, C851, C852, C853, C854, C855, C856, C857, C858, C859, C860, C861, C862, C863, C864, C865, C866, C867, C868, C869, C870, C871, C872, C873, C874, C875, C876, C877, C878, C879,

105. The compound of claim 1, wherein the compound is selected from the group consisting of a compound identified as C880, C881, C882, C883, C884, C885, C886, C887, C888, C889, C890, C891, C892, C893, C894, C895, C896, C897, C898, C899,

106. A pharmaceutical composition, comprising a therapeutically effective amount of a compound of claim 1 and a pharmaceutically acceptable carrier, excipient, or diluent.

107. A method of treating or preventing a disorder or condition selected from the group consisting of obesity, an obesity-associated disorder, a metabolic disorder, metabolic syndrome, an endocrine disorder, an appetite disorder, an eating disorder, an eating disorder requiring appetite control, atherosclerosis, diabetes, diabetes mellitus, high cholesterol, hyperlipidemia, cachexia, anorexia, bulimia, inflammation, a chronic inflammatory disorder, rheumatoid arthritis, asthma, psoriasis, a cardiovascular disorder, angina, cardiac ischemia, cardiac failure, heart disease, congestive heart failure, ischemic heart disease, chronic heart disease, hemorrhagic shock, septic shock, cirrhosis, a neurological disorder, anxiety, depression, an attention deficit disorder, a memory disorder, a cognitive disorder, a gastrointestinal disorder, reduced gastric motility, reduced gastric and intestinal motility, excessive gastric motility, post-operative gastric ileus, delayed gastric emptying, delayed gastric emptying due to diabetes, delayed gastric emptying post-operatively, short bowel syndrome, a gastric ulcer, nausea, emesis, diarrhea, gastroparesis, diabetic gastroparesis, opioid-induced bowel dysfunction, chronic intestinal pseudoobstruction, a sleep disorder, insomnia, a hyperproliferative disorder, cancer, cancer cachexia, dwarfism, osteoporosis, a catabolic state, somatopause, osteopenia, a disorder of the pancreas, a hormone deficiency, gastrointestinal dumping syndrome, postgastroenterectomy syndrome, celiac disease, AIDS, wasting, age-related decline in body composition, hypertension, retinopathy, dyslipidemia, a gall stone, osteoarthritis, congestive heart failure, insulin resistance, burn, wound, protein loss, sexual dysfunction, a central nervous system disorder, a genetic disorder, irritable bowel syndrome (IBS), non-ulcer dyspepsia, Crohn's disease, a gastroesophogeal reflux disorder, constipation, ulcerative colitis, pancreatitis, infantile hypertrophic pyloric stenosis, carcinoid syndrome, malabsorption syndrome, atrophic colitis, gastritis, gastric stasis, frailty, acromegaly, and protein loss comprising administering to a subject a pharmaceutically effective amount of a compound of claim 1.

108. The method of claim 107, wherein said compound alleviates or treats a disorder or condition by modulating, agonizing, inverse agonizing, or antagonizing a ghrelin receptor.

109. The method of claim 107, wherein said compound alleviates or treats a disorder or condition by inverse agonizing or antagonizing a ghrelin receptor.

110. The method of claim 107, wherein the disorder or condition is a disorder selected from the group consisting of a neurological disorder, anxiety, depression, an attention deficit disorder, a memory disorder, and a cognitive disorder.

111. The method of claim 107, wherein the disorder or condition is selected from the group consisting of obesity, metabolic syndrome, an appetite disorder, an eating disorder, an eating disorder requiring appetite control, atherosclerosis, diabetes, heart disease, high cholesterol, hyperlipidemia, cachexia, anorexia, and bulimia.

112. The method of claim 107, wherein the disorder or condition is a sleep disorder.

113. The method of claim 112, wherein the sleep disorder is insomnia or narcolepsy.

114. The method of claim 107, wherein the disorder or condition is selected from the group consisting of a reduced gastric motility, reduced gastric and intestinal motility, excessive gastric motility, post-operative gastric ileus, delayed gastric emptying, delayed gastric emptying due to diabetes, delayed gastric emptying post-operatively, short bowel syndrome, a gastric ulcer, nausea, emesis, diarrhea and a gastrointestinal disorder.

115. The method of claim 107, wherein the inflammation is caused by a disorder or condition selected from the group consisting of a chronic inflammatory disorder, rheumatoid arthritis, asthma, an allergy, and psoriasis.

116. The method of claim 107, wherein the disorder or condition is selected from the group consisting of a cardiovascular disorder, angina, cardiac ischemia, cardiac failure, heart disease, hemorrhagic shock, septic shock, and cirrhosis.

117. The method of claim 107, wherein the disorder or condition is selected from the group consisting of dwarfism, osteoporosis, a catabolic state, somatopause, and osteopenia.

118. The method of claim 107, wherein the disorder or condition is a hyperproliferative disorder or cancer.

119. The method of claim 107, wherein the disorder or condition is a disorder of the pancreas.

120. The method of claim 107, wherein the disorder or condition is a hormone deficiency.

121. A method of treating or alleviating obesity comprising administering to a subject a therapeutically effective amount of a compound of claim 1.

122. A method of alleviating or controlling a symptom associated with an eating disorder comprising administering to a subject a therapeutically effective amount of a compound of claim 1.

123. The method of claim 122, wherein the symptom is increased appetite or binge eating.

124. A method of promoting weight loss in a subject comprising administering to the subject a therapeutically effective amount of a compound of claim 1.

125. A method of preventing weight gain in a subject comprising administering to a subject a therapeutically effective amount of a compound of claim 1.

126. The method of claim 125, wherein the subject is taking a medication selected from the group consisting of insulin, thiazolidinedione, sulfonylurea, corticosteroid, progestational steroid, antihistamine, alpha-adrenergic blocker, beta-adrenergic blocker, an antidepressant, antipsychotic, and anticonvulsant.

127. A method of preventing weight loss in a subject comprising administering to a subject a therapeutically effective amount of a compound of claim 1.

128. The method of claim 127, wherein the weight loss is caused by chemotherapy, radiation therapy, temporary immobilization, permanent immobilization or dialysis.

129. A method for maintaining the weight of a subject comprising administering a therapeutically effective amount of a compound of claim 1.

130. A method of improving sleep architecture, facilitating induction of sleep, or improving the quality of sleep of a subject comprising administering to the subject a therapeutically effective amount of a compound of claim 1.

131. The method of claim 130, further comprising a sleep agent.

132. A method for maintaining the sleep of a subject comprising administering a therapeutically effective amount of a compound of claim 1.

133. The method of claim 132, further comprising a sleep agent.

134. A method for facilitating alertness or awakefulness of a subject comprising administering a therapeutically effective amount of a compound of claim 1.

135. The method of claim 134, wherein the subject is taking an agent that causes drowsiness or induces sleep.

136. A method of controlling the level of glucose in a subject comprising administering to the subject a therapeutically effective amount of a compound of claim 1.

137. A method of treating cancer comprising administering to a subject a therapeutically effective amount of a compound of claim 1.

138. A method of treating diabetes comprising administering to a subject a therapeutically effective amount of a compound of claim 1.

139. A method of preventing or alleviating inflammation comprising administering to a subject a therapeutically effective amount of a compound of claim 1.

140. The method of claim 139, wherein the inflammation is caused by a chronic inflammatory disease, rheumatoid arthritis, asthma, an allergy, or psoriasis.

141. A method of diagnosing a hormone deficiency comprising administering to a subject a therapeutically effective amount of a compound of claim 1.

142. A method of modulating production of a hormone comprising administering to a subject a therapeutically effective amount of a compound of claim 1.

143. A method of improving the memory of a subject comprising administering to the subject a therapeutically effective amount of a compound of claim 1.

144. A method of alleviating or treating a symptom associated with a neurological disorder comprising administering to a subject with altered cognition a therapeutically effective amount of a compound of claim 1.

145. A method for treating post-operative ileus or cachexia comprising administering to a subject with altered cognition a therapeutically effective amount of a compound of claim 1.

146. The method of claim 145, wherein the post-operative ileus or cachexia is caused by cancer, AIDS, a cardiac disease, a renal disease, or gastroparesis.

147. A method of modulating, agonizing, inverse agonizing, or antagonizing a ghrelin receptor comprising administering to a subject a therapeutically effective amount of a compound of claim 1.
